(19) United States
(12) Patent Application Publication
(10) Pub. No.: US 2026/0355323 A1
(43) Pub. Date: Nov. 12, 2026

(54) CEREBLON-RECRUITING BCL-XL/BCL-2 DUAL DEGRADERS

(71) Applicants: University of Florida Research Foundation, Incorporated, Gainesville, FL (US); Board of Regents, The University of Texas System, Austin, TX (US)

(72) Inventors: Wanyi Hu, Gainesville, FL (US); Peiyi Zhang, Gainesville, FL (US); Guangrong Zheng, Gainesville, FL (US); Daohong Zhou, Waldo, FL (US)

(73) Assignees: University of Florida Research Foundation, Incorporated, Gainesville, FL (US); Board of Regents, The University of Texas System, Austin, TX (US)

(21) Appl. No.: 19/117,342

(22) PCT Filed: Oct. 3, 2023
(86) PCT No.: PCT/US2023/075887

§ 371 Date: Mar. 31, 2025

Related U.S. Application Data
(60) Provisional application No. 63/412,834, filed on Oct. 3, 2022.

(51) Int. Cl. A61K 47/55; A61K 45/06; A61K 47/54; A61P 35/02

(52) U.S. Cl. CPC A61K 47/55; A61K 45/06; A61K 47/545; A61P 35/02

(57) ABSTRACT

The present disclosure provides compounds of Formula (I), which may be Bcl-xL and Bcl-2 dual inhibitors. The compounds may target and degrade Bcl-xL and Bcl-2. The present disclosure also provides pharmaceutical compositions, kits, and methods of using the compounds for treating a disease (e.g., cancer).

### RELATED APPLICATIONS

This application claims priority under 35 U.S.C. § 119 (e) to U.S. Provisional Application No. 63/412,834, filed Oct. 3, 2022, which is incorporated herein by reference.

### GOVERNMENT SUPPORT INFORMATION

This invention was made with government support under Grant Nos. R01 CA242003 and R01 CA241191 awarded by the National Institutes of Health. The government has certain rights in the invention.

### BACKGROUND

The B-cell lymphoma 2 (Bcl-2) protein family, consisting of pro- and anti-apoptotic members, plays a critical role in determining cell fate through regulation of the intrinsic apoptosis pathway. The anti-apoptotic Bcl-2 family proteins, such as Bcl-2, Bcl-xL, Bcl-w, and Mcl-1, are upregulated in many cancers and associated with tumor initiation, progression, and resistance to chemo- and targeted therapies. Thus, these anti-apoptotic Bcl-2 proteins are attractive targets for the development of novel anti-cancer agents (Lessene et al., Nat Rev Drug Discov 7:989-1000, 2008; Vogler et al., Cell Death Differ 2009; 16:360-367; Delbridge et al., Nat Rev Cancer 16:99-109, 2016). Numerous Bcl-2 small molecule inhibitors have been reported (Bajwa et al., Expert Opin Ther Patents 22:37-55, 2012; Vogler, Adv Med. 1-14, 2014; Ashkenazi et al., 16:273-284, 2017). The following are some of the Bcl-2 small molecule inhibitors that have been investigated at various stages of drug development: ABT-737 (US20070072860), navitoclax (ABT-263, WO2009155386), venetoclax (ABT-199, WO2010138588), obatoclax (GX 15-070, WO2004106328), (-)-gossypol (AT-101, WO2002097053), sabutoclax (BI-97C1, WO2010120943), TW-37 (WO2006023778), BM-1252 (APG-1252), and A-1155463 (VV02010080503).

Venetoclax, a selective Bcl-2 inhibitor, was approved by the FDA in 2016 for the treatment of chronic lymphocytic leukemia (CLL) with 17-p deletion. Venetoclax was designed to have high selectivity for Bcl-2 over Bcl-xL to avoid the on-target platelet toxicity (Souers et al., Nat Med 19:202-208, 2013). Platelets depend on Bcl-xL to maintain their viability, therefore dose-limiting thrombocytopenia has been observed in animals and/or humans treated with ABT-737 (Schoenwaelder et al., Blood 118:1663-1674, 2011), ABT-263 (Tse et al., Cancer Res 68:3421-3428, 2008; Roberts et al., Bri J Haematol 170:669-678, 2015), BM-1197 (Bai et al., PLOS ONE 9:e99404, 2014), or A-1155463 (Tao et al., ACS Med Chem Lett 5:1088-1093,2014), due to their inhibition of Bcl-xL. However, many CLL patients are resistant to venetoclax (Roberts et al., N Engl J Med 374:311-322, 2016) and upregulation of Bcl-xL by microenvironmental survival signals has been identified as the major component accountable for the resistance, consistent with the high efficacy of Bcl-2/Bcl-xL dual inhibitor ABT-263 in killing venetoclax resistant CLL cells (Oppermann et al., Blood 128:934-947, 2016). In addition, Bcl-xL is generally more frequently overexpressed than Bcl-2 in solid tumors. Importantly, promising results have been documented from preclinical and clinical studies of ABT-263, as a single-agent or in combination with other antitumor agents, against several solid and hematologic malignancies (Delbridge et al., Nat Rev Cancer 16:99-109, 2016). Therefore, it is highly desirable to develop a strategy that can retain the antitumor versatility and efficacy of the Bcl-xL/Bcl-2 dual inhibitors, while sparing their on-target platelet toxicity.

Thus, there is a need to develop compounds that can retain the antitumor versatility and efficacy of the Bcl-xL/Bcl-2 dual inhibitors, while avoiding their on-target platelet toxicity.

### SUMMARY OF THE DISCLOSURE

In one aspect, the present disclosure provides a compound of Formula (I), or a pharmaceutically acceptable salt, solvate, hydrate, polymorph, co-crystal, tautomer, stereoisomer, isotopically labeled compound, or prodrug thereof:

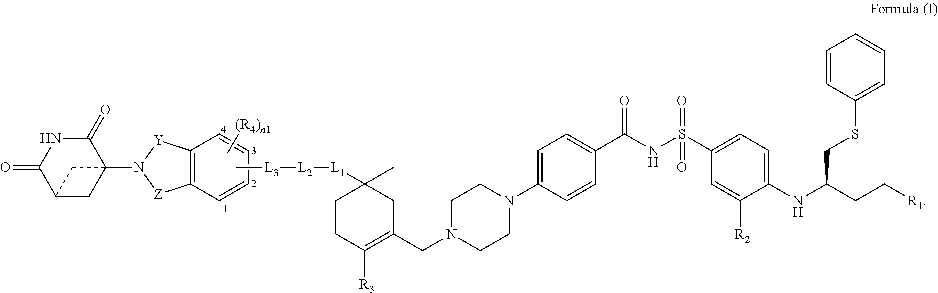

In certain embodiments, the compounds include a Bcl-xL and/or Bcl-2 binding moiety

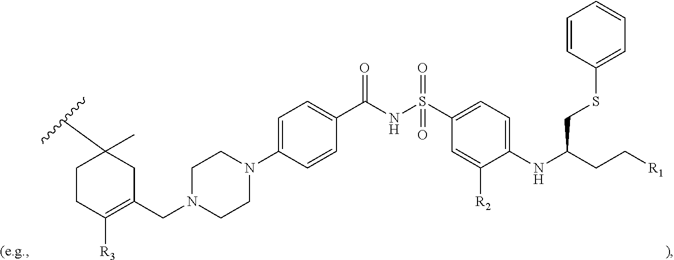

a cereblon (CRBN) binding moiety

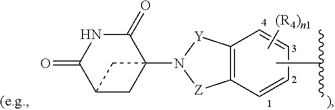

and a linker (e.g., -L₃-L₂-L₁-) connecting the Bcl-xL and/or Bcl-2 binding moiety and the CRBN binding moiety.

Compared to known Bcl-xL and/or Bcl-2 inhibitors (e.g., ABT-263:

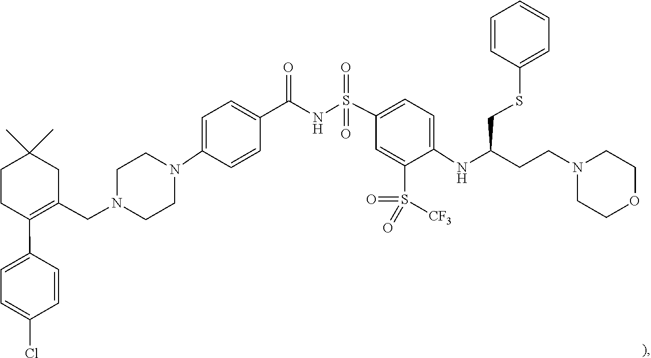 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

the compounds and pharmaceutical compositions provided herein may have unexpected advantages (e.g., increased potency and/or efficacy in inhibiting the activity and/or production of Bcl-xL and/or Bcl-2, increased bioavailability, reduced toxicity, increased safety, increased therapeutic window, reduced resistance, and/or increased subject compliance). In certain embodiments, the linker is shorter and/or more rigid than known linkers.

The compounds may promote the degradation of the anti-apoptotic Bcl-2 family of proteins. The compounds may also promote the degradation of the anti-apoptotic Bcl-xL family of proteins. The compounds may be Bcl-xL/Bcl-2 dual inhibitors. The compounds may connect a Bcl-2 or Bcl-xL small molecule inhibitor or ligand to an E3 ligase binding moiety, such as CRBN. In humans, CRBN is a protein encoded by the CRBN gene (Ensembl: ENSG00000113851). In certain embodiments, CRBN forms an E3 ubiquitin ligase complex with damaged DNA binding protein 1 (DDB1), Cullin-4A (CUL4A), and regulator of cullins 1 (ROC1). The compounds may actively recruit anti-apoptotic Bcl-2 family of proteins to E3 ligase, resulting in their degradation by ubiquitin proteasome system.

Platelets depend on Bcl-xL protein for survival. Thus, inhibition of Bcl-xL protein in platelets causes thrombocytopenia which limits the use of Bcl-xL inhibitors as cancer therapeutic agents. Given the well-documented importance of Bcl-xL in solid tumors and its contribution to drug resistance, strategies devised to minimize the on-target platelet toxicity associated with the inhibition of Bcl-xL could boost the therapeutic applications of drugs like ABT-263, a dual Bcl-2/Bcl-xL inhibitor, in cancer. The compounds of the present disclosure were designed to recruit CRBN E3 ligase, which is minimally expressed in platelets for the targeted degradation of Bcl-xL.

Thus, the compounds provided herein may reduce platelet toxicity compared with their corresponding Bcl-2/Bcl-xL inhibitors. Accordingly, the present disclosure provides compounds, pharmaceutical compositions, kits, and methods for at least selectively degrading anti-apoptotic Bcl-2 family of proteins.

In another aspect, the present disclosure provides a pharmaceutical composition comprising a compound of the present disclosure, and a pharmaceutically acceptable carrier.

In another aspect, the present disclosure provides a kit comprising a provided compound or pharmaceutical composition, and instructions for using the compound or pharmaceutical composition.

In another aspect, the present disclosure provides a method of degrading a Bcl-2 protein in a cell, tissue, or biological sample, the method comprising contacting the cell, tissue, or biological sample with an effective amount of a compound or pharmaceutical composition provided herein.

In another aspect, the present disclosure provides a method of degrading a Bcl-xL protein in a cell, tissue, or biological sample, the method comprising contacting the cell, tissue, or biological sample with an effective amount of a compound or pharmaceutical composition provided herein.

In another aspect, the present disclosure provides a method of treating a disease in a subject in need thereof, the method comprising administering to the subject an effective amount of a compound or pharmaceutical composition provided herein.

In another aspect, the present disclosure provides a method of preventing a disease in a subject in need thereof, the method comprising administering to the subject an effective amount of a compound or pharmaceutical composition provided herein.

In another aspect, the present disclosure provides a method of selectively killing one or more cancer cells in a tissue or biological sample, the method comprising contacting the tissue or biological sample with an effective amount of a compound or pharmaceutical composition of the present disclosure

In another aspect, the present disclosure provides a method of selectively killing one or more cancer cells in a subject in need thereof, the method comprising administering to the subject an effective amount of a compound or pharmaceutical composition of the present disclosure.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ₁₄C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₄, C₄₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). In some embodiments, the one or more carbon-carbon double bonds is internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In some embodiments, in an alkenyl group, a C═C double bond for which the stereochemistry is not specified (e.g., —CH═CHCH₃ or

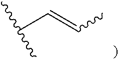

is in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). In some embodiments, the one or more carbon-carbon triple bonds is internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. 30 Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and is saturated or contains one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In some embodiments, in heterocyclyl groups that contain one or more nitrogen atoms, the point of attachment is a carbon or nitrogen atom, as valency permits. In some embodiments, a heterocyclyl group is either monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and is either saturated or contains one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In some embodiments, in heteroaryl groups that contain one or more nitrogen atoms, the point of attachment is a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. In some embodiments, in polycyclic heteroaryl groups wherein one ring does not contain a heteroatom (e.g., indolyl, quinolinyl, carbazolyl, and the like) the point of attachment is on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Carbon atoms of the groups and moieties described herein are substituted or unsubstituted, as valency permits. Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})³, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR)₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl 30 or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO2R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is 10 independently substituted with 0, 1, 2, 3, 4, or 5 R^{gg} groups, or two geminal R^{dd} 12 substituents are joined to form ═O or ═S;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl 22 or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂ (C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S) NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents are joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

Nitrogen atoms of the groups and moieties described herein are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, 5-14 membered heteroaryl, or oxo, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(—NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene 2 and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitrophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido) benzyl carbamate, 1,1-dimethyl-3-(N,N-dimethylcarboxamido) propyl carbamate, 1,1-dimethylpropynyl carbamate, di(2-pyridyl) methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium) benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl) amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene) amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl) amine, N-borane derivatives, N-diphenylborinic acid derivatives, N-[phenyl (pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

Oxygen atoms of the groups and moieties described herein are substituted or unsubstituted as valency permits. In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain 30 embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR)₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. 10 Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy) methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-20 methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, 26 p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di (p-methoxyphenyl)phenylmethyl, tri (p-methoxyphenyl)methyl, 4-(4′-bromophenacyloxyphenyl)diphenylmethyl, 4,4′,4″-tris (4,5-dichlorophthalimidophenyl)methyl, 4,4′,4″-tris (levulinoyloxyphenyl)methyl, 4,4′,4″-tris (benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl) carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy) ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

Sulfur atoms of the groups and moieties described herein are substituted or unsubstituted as valency permits. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group. In certain embodiments, at least one sulfur atom substituent is OXO.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. 14

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. In some embodiments, an anionic counterion is monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄], B(C₆F₅)₄⁻, BPh₄, Al(OC(CF₃)₃)₄, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group-OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR)₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group-SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR)₂, and —NHP(═O)(N(R^{bb})₂)₂, wherein R^{aa}, R^{bb} and R^{cc} are as defined herein, and wherein R^{bb} of the group-NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR)₂, and —NR^{bb}P(═O)(N(R^{bb})₂)₂, wherein R^{aa}, R^{bb}, and R^{e} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfony” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein. The term “acyl” refers to a group having the general formula —C(═O)R^{aa}, —C(═O)OR^{aa}—C(═O)—O—C(═O)R^{aa}, —C(═O)SR^{aa}, —C(═O)N(R^{bb})₂, —C(═S) R^{aa}, —C(═S)N(R^{bb})₂, and —C(═S)S(R^{aa}), —C(═NR^{bb})R^{aa}, —C(═NR^{bb})OR^{aa}, —C(═NR^{bb}) SR^{aa}, and —C(═NR^{bb})N(R^{bb})₂, wherein R^{aa} and R^{bb} are as defined herein. In some embodiments, the term “acyl” refers to a group having the general formula —C(═O)R^{aa}, —C(═O)OR^{aa}, —C(═O)—O—C(═O)R^{aa}, —C(═O)SR^{aa}, or —C(═O)N(R^{bb})₂.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), C(═NR^{bb})N(R^{bb})₂), wherein R^{aa} and R^{bb} are as defined herein.

The term “silyl” refers to the group —Si(R^{aa})₃, wherein R^{aa} is as defined herein.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances.

A “non-hydrogen group” refers to any group that is defined for a particular variable that not hydrogen.

The term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. In some embodiments, the compounds provided herein are prepared, e.g., in crystalline form. In some embodiments, the compounds provided herein are prepared, e.g., in crystalline form, and are solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound, or a salt thereof, that is associated with water. Typically, the number of the water molecules contained in a hydrate of a compound, or a salt thereof, is in a definite ratio to the number of the compound molecules in the hydrate. Therefore, in some embodiments, a hydrate of a compound, or a salt thereof, is represented, for example, by the general formula R·x H₂O, wherein R is the compound, or a salt thereof, and x is a number greater than 0. A given compound, or a salt thereof, may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. In some embodiments, an enantiomer is characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. In some embodiments, co-crystals are useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. In some embodiments, various polymorphs of a compound (or a salt, hydrate, or solvate thereof) are prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy) alkyl esters or ((alkoxycarbonyl)oxy) alkylesters. In some embodiments, C₁-C₈ alkyl, C₂-C₈ alkenyl, C₂-C₈ alkynyl, aryl, C₇-C₁₂ substituted aryl, and C₇-C₁₂ arylalkyl esters of the compounds described herein are preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal. The term “patient” refers to a human subject in need of treatment of a disease.

The term “tissue” refers to any biological tissue of a subject (including a group of cells, a body part, or an organ) or a part thereof, including blood and/or lymph vessels. In some embodiments, “tissue” is the object to which a compound, particle, and/or composition of the disclosure is delivered. In some embodiments, a tissue is an abnormal or unhealthy tissue, which may need to be treated. A tissue may also be a normal or healthy tissue that is under a higher than normal risk of becoming abnormal or unhealthy, which may need to be prevented.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound provided herein, a compound useful in a provided method, or a pharmaceutical composition provided herein, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment is administered after one or more signs or symptoms of the disease have developed or have been observed. In other embodiments, treatment is administered in the absence of signs or symptoms of the disease. For example, in some embodiments, treatment is administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for preventing a disease.

The term “inhibit” or “inhibition”, for example, in the context of a Bcl-xL or Bcl-2, refers to a reduction in activity or production. In some embodiments, the term refers to a reduction of the level of activity and/or production, e.g., Bcl-xL or Bcl-2 activity and/or production, to a level that is statistically significantly lower than an initial level, which may, for example, be a baseline level of activity and/or production. In some embodiments, the term refers to a reduction of the level of activity and/or production, e.g., Bcl-xL or Bcl-2 activity and/or production, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity and/or production.

The term “Bcl-2 family” refers to a family consists of a number of evolutionarily-conserved proteins that share Bcl-2 homology (BH) domains. The Bcl-2 family may be able to regulate apoptosis at the mitochondrion. The Bcl-2 family may promote or inhibit apoptosis, or control apoptosis by governing mitochondrial outer membrane permeabilization (MOMP), which is a key step in the intrinsic pathway of apoptosis. In certain embodiments, the Bcl-2 family is a human Bcl-2 family.

The term “Bcl-2” refers to a protein of the Bcl-2 family and, when in humans, is encoded by the BCL2 gene.

The term “Bcl-xL” refers to a protein of the Bcl-2 family and, when in humans, is encoded by the BCL2-like 1 gene.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25^{th} ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); achoriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenia purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

The term “psychiatric disorder” refers to a condition or disorder relating to the functioning of the brain and the cognitive processes or behavior. Psychiatric disorders may be further classified based on the type of neurological disturbance affecting the mental faculties. Psychiatric disorders are expressed primarily in abnormalities of thought, feeling, emotion, and/or behavior producing either distress or impairment of function (for example, impairment of mental function such with dementia or senility). The term “psychiatric disorder” is, accordingly, sometimes used interchangeably with the term “mental disorder” or the term “mental illness”.

A psychiatric disorder is often characterized by a psychological or behavioral pattern that occurs in an individual and is thought to cause distress or disability that is not expected as part of normal development or culture. Definitions, assessments, and classifications of mental disorders can vary, but guideline criteria listed in the International Classification of Diseases and Related Health Problems (ICD, published by the World Health Organization, WHO), or the Diagnostic and Statistical Manual of Mental Disorders (DSM, published by the American Psychiatric Association, APA) and other manuals are widely accepted by mental health professionals. Individuals may be evaluated for various psychiatric disorders using criteria set forth in these and other publications accepted by medical practitioners in the field and the manifestation and severity of a psychiatric disorder may be determined in an individual using these publications.

Categories of diagnoses in these schemes may include dissociative disorders, mood disorders, anxiety disorders, psychotic disorders, eating disorders, developmental disorders, personality disorders, and other categories. There are different categories of mental disorder, and many different facets of human behavior and personality that can become disordered.

One group of psychiatric disorders includes disorders of thinking and cognition, such as schizophrenia and delirium. A second group of psychiatric disorders includes disorders of mood, such as affective disorders and anxiety. A third group of psychiatric disorders includes disorders of social behavior, such as character defects and personality disorders. And a fourth group of psychiatric disorders includes disorders of learning, memory, and intelligence, such as mental retardation and dementia. Accordingly, psychiatric disorders encompass schizophrenia, delirium, attention deficit disorder (ADD), schizoaffective disorder, depression (e.g., lithium-resistant depression), mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder and disinhibition.

Some diseases classified as neurodegenerative diseases, for example Alzheimer's disease, also sometimes show aspects of psychiatric disorders as listed herein, for example disorders of memory or dementia. Some neurodegenerative diseases or manifestations thereof can, accordingly, also be referred to as psychiatric disorders. These terms are, therefore, not mutually exclusive.

The state of anxiety or fear can become disordered, so that it is unusually intense or generalized over a prolonged period of time. Commonly recognized categories of anxiety disorders include specific phobia, generalized anxiety disorder, social anxiety disorder, panic disorder, agoraphobia, obsessive-compulsive disorder, post-traumatic stress disorder.

Relatively long lasting affective states can also become disordered. Mood disorder involving unusually intense and sustained sadness, melancholia or despair is known as clinical depression (or major depression), and may more generally be described as emotional dysregulation. Milder but prolonged depression can be diagnosed as dysthymia. Bipolar disorder involves abnormally “high” or pressured mood states, known as mania or hypomania, alternating with normal or depressed mood.

Patterns of belief, language use and perception can become disordered. Psychotic disorders centrally involving this domain include schizophrenia and delusional disorder. schizoaffective disorder is a category used for individuals showing aspects of both schizophrenia and affective disorders. Schizotypy is a category used for individuals showing some of the traits associated with schizophrenia but without meeting cut-off criteria.

The fundamental characteristics of a person that influence his or her cognitions, motivations, and behaviors across situations and time—can be seen as disordered due to being abnormally rigid and maladaptive. Categorical schemes list a number of different personality disorders, such as those classed as eccentric (e.g., paranoid personality disorder, schizoid personality disorder, schizotypal personality disorder), those described as dramatic or emotional (antisocial personality disorder, Borderline personality disorder, histrionic personality disorder, narcissistic personality disorder) or those seen as fear-related (avoidant personality disorder, dependent personality disorder, obsessive-compulsive personality disorder).

### BRIEF DESCRIPTION OF THE DRAWINGS

The accompanying drawings, which constitute a part of this specification, illustrate several embodiments of the present disclosure and together with the description, serve to explain the principles of the present disclosure.

FIGS. 1A to 1C: Compounds #1, #4, #11, #12, #44, #128 and #133-135 degrade Bcl-xL in a dose-dependent manner in Jurkat and Hela cells, and some of these compounds also slightly reduce Bcl-2 protein levels in both Jurkat and Hela cells. However, none of these compounds degrades Bcl-w in Hela cells. FIG. 1A: immunoblot analysis of Bcl-xL and Bcl-2 expression in Jurkat cells after treatment with increasing concentrations of the compound as indicated for hours. FIG. 1B: immunoblot analysis of Bcl-xL and Bcl-2 expression in Hela cells after treatment with increasing concentrations of the compound as indicated for 16 hours. FIG. 1C: immunoblot analysis of Bcl-XL, Bcl-2 and Bcl-w expression in Hela cells after treatment with increasing concentrations of the compound as indicated for 16 hours.

FIG. 2: immunoblot analysis of Bcl-xL, Bcl-2 and Bcl-w expression in WI-38 cells after treatment of the compounds as indicated for 16 hours. Most of the compounds reduced Bcl-xL and Bcl-w protein levels in WI-38 cells at 100 nM, and some of them also slightly degraded Bcl-2 protein.

### DETAILED DESCRIPTION

### Compounds

In one aspect, the present disclosure provides a compound of Formula (I), or a pharmaceutically acceptable salt, solvate, hydrate, polymorph, co-crystal, tautomer, stereoisomer, isotopically labeled compound, or prodrug thereof:

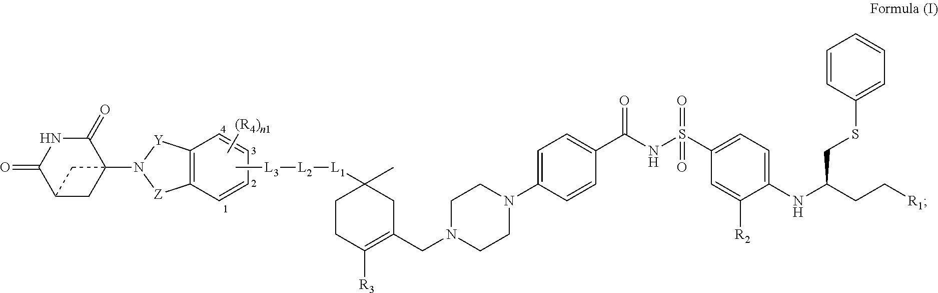

wherein:
   R₁ is —O(substituted or unsubstituted alkyl) or —N(R^{a})₂, wherein each instance of R^{a} is independently substituted or unsubstituted alkyl or substituted or unsubstituted carbocyclyl, or two instances of R^{a} are joined with the nitrogen atom to which they are attached to form substituted or unsubstituted heterocyclyl;
   R₂ is SO₂CF₃ or NO₂;
   R₃ is

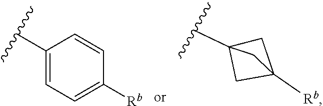

wherein R^{b} is halogen or substituted or unsubstituted alkyl;
each instance of R₄ is independently F, Cl, —CN, —CFH₂, —CF₂H, —CF₃, or —OCH₃;
n1 is 0, 1, 2, or 3;
Z is a bond, CH₂, or CO;
Y is CO;
or

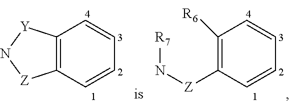

wherein R₆ is F, Cl, —CN, —CFH₂, —CF₂H, —CF₃, —OCH₃, or H; and R₇ is H or substituted or unsubstituted alkyl;
L₁ is substituted or unsubstituted, C₁₋₁₁ alkylene, substituted or unsubstituted, C₂₋₁₁ alkenylene, substituted or unsubstituted, C₂₋₁₁ alkynylene, or substituted or unsubstituted heterocyclylene, optionally wherein one or more backbone carbon atoms in the substituted or unsubstituted, C₁₋₁₁ alkylene, substituted or unsubstituted, C₂₋₁₁ alkenylene, or substituted or unsubstituted, C₂₋₁₁ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene;
each instance of R₅ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L₂ is substituted or unsubstituted, C₁₋₁₂ alkylene, substituted or unsubstituted, C₂₋₁₂ alkenylene, substituted or unsubstituted, C₂₋₁₂ alkynylene, substituted or unsubstituted heterocyclylene, or substituted or unsubstituted carbocyclylene, optionally wherein one or more backbone carbon atoms in the substituted or unsubstituted, C₁₋₁₂ alkylene, substituted or unsubstituted, C₂₋₁₂ alkenylene, or substituted or unsubstituted, C₂₋₁₂ alkynylene are independently replaced with —O—, —NR₅—, —N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene; and
L₃ is a bond, substituted or unsubstituted, C₁₋₁₃ alkylene, substituted or unsubstituted, C₂₋₁₃ alkenylene, substituted or unsubstituted, C₂₋₁₃ alkynylene, or substituted or unsubstituted heterocyclylene, optionally wherein one or more backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene.

In certain embodiments, the present disclosure provides a compound of Formula (I), or a pharmaceutically acceptable salt, solvate, hydrate, polymorph, co-crystal, tautomer, stereoisomer, isotopically labeled compound, or prodrug thereof, wherein:
R₁ is —O(substituted or unsubstituted alkyl) or —N(R^{a})₂, wherein each instance of R^{a} is independently substituted or unsubstituted alkyl or substituted or unsubstituted carbocyclyl, or two instances of R^{a} are joined with the nitrogen atom to which they are attached to form substituted or unsubstituted heterocyclyl;
R₂ is NO₂ or SO₂CF₃;
R₃ is

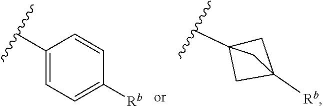

wherein R^{b} is halogen or substituted or unsubstituted alkyl;
each instance of R⁴ is independently F, Cl, —CN, —CFH₂, —CF₂H, —CF₃, or —OCH₃;
n1 is 0, 1, 2, or 3;
Z is a bond, CH₂, or CO;
Y is CO;
or

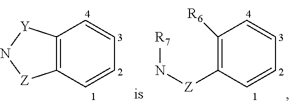

wherein R₆ is F, Cl, —CN, —CFH₂, —CF₂H, —CF₃, or —OCH₃, and R₇ is H or substituted or unsubstituted alkyl;
L₁ is substituted or unsubstituted, C₁₋₁₁ alkylene, substituted or unsubstituted, C₂₋₁₁ alkenylene, substituted or unsubstituted, C₂₋₁₁ alkynylene, or substituted or unsubstituted heterocyclylene, optionally wherein one or more backbone carbon atoms in the substituted or unsubstituted, C₁₋₁₁ alkylene, substituted or unsubstituted, C₂₋₁₁ alkenylene, or substituted or unsubstituted, C₂₋₁₁ alkynylene are independently replaced with —O—, —NR₅-, —N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene;
each instance of R⁵ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L₂ is substituted or unsubstituted, C₁₋₁₂ alkylene, substituted or unsubstituted, C₂₋₁₂ alkenylene, substituted or unsubstituted, C₂₋₁₂ alkynylene, substituted or unsubstituted heterocyclylene, or substituted or unsubstituted carbocyclylene, optionally wherein one or more backbone carbon atoms in the substituted or unsubstituted, C₁₋₁₂ alkylene, substituted or unsubstituted, C₂₋₁₂ alkenylene, or substituted or unsubstituted, C₂₋₁₂ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene; and
L₃ is substituted or unsubstituted, C₁₋₁₃ alkylene, substituted or unsubstituted, C₂₋₁₃ alkenylene, substituted or unsubstituted, C₂₋₁₃ alkynylene, or substituted or unsubstituted heterocyclylene, optionally wherein one or more backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene.

In certain embodiments, R₆ is F, Cl, —CN, —CFH₂, —CF₂H, —CF₃, or —OCH₃, and
L₃ is substituted or unsubstituted, C₁₋₁₃ alkylene, substituted or unsubstituted, C₂₋₁₃ alkenylene, substituted or unsubstituted, C₂₋₁₃ alkynylene, or substituted or unsubstituted heterocyclylene, optionally wherein one or more backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene.

In certain embodiments, R₁ is —O(substituted or unsubstituted alkyl). In certain embodiments, R₁ is —O(unsubstituted alkyl). In certain embodiments, R₁ is —O(unsubstituted C₁₋₆ alkyl). In certain embodiments, R₁ is —O(substituted alkyl). In certain embodiments, R₁ is —O(substituted C₁₋₆ alkyl). In certain embodiments, R₁ is —O(alkyl substituted at least with substituted or unsubstituted heterocyclyl). In certain embodiments, R₁ is —O(alkyl substituted at least with substituted or unsubstituted heterocyclyl, wherein at least one heteroatom in the heterocyclic ring system is nitrogen). In certain embodiments, R₁ is —O(alkyl substituted at least with substituted or unsubstituted, monocyclic heterocyclyl). In certain embodiments, R₁ is —O(alkyl substituted at least with substituted or unsubstituted, monocyclic heterocyclyl comprising only carbon and nitrogen atoms in the heterocyclic ring system).

In certain embodiments, R₁ is —N(R^{a})₂. In certain embodiments, R₁ is —N(R^{a})₂, wherein each instance of R^{a} is independently substituted or unsubstituted alkyl or substituted or unsubstituted carbocyclyl. In certain embodiments, R₁ is —N(R^{a})₂, wherein at least one instance of R^{a} is unsubstituted C₁₋₆ alkyl. In certain embodiments, R₁ is —N(R^{a})₂, wherein at least one instance of R^{a} is substituted C₁₋₆ alkyl. In certain embodiments, R₁ is —N(R^{a})₂, wherein at least one instance of R^{a} is C₁₋₆ alkyl substituted at least with —OH. In certain embodiments, R₁ is —N(R^{a})₂, wherein at least one instance of R^{a} is substituted or unsubstituted carbocyclyl. In certain embodiments, R₁ is —N(R^{a})₂, wherein at least one instance of R^{a} is unsubstituted monocyclic saturated carbocyclyl.

In certain embodiments, R₁ is —N(R^{a})₂, wherein two instances of R^{a} are joined with the nitrogen atom to which they are attached to form substituted or unsubstituted heterocyclyl. In certain embodiments, R₁ is —N(R^{a})₂, wherein two instances of R^{a} are joined with the nitrogen atom to which they are attached to form substituted or unsubstituted, monocyclic heterocyclyl. In certain embodiments, R₁ is —N(R^{a})₂, wherein two instances of R^{a} are joined with the nitrogen atom to which they are attached to form substituted or unsubstituted, monocyclic heterocyclyl comprising only carbon and nitrogen atoms in the heterocyclic ring system. In certain embodiments, R₁ is —N(R^{a})₂, wherein two instances of R^{a} are joined with the nitrogen atom to which they are attached to form substituted or unsubstituted, monocyclic heterocyclyl comprising only carbon, nitrogen, and oxygen atoms in the heterocyclic ring system. In certain embodiments, R₁ is —N(R^{a})₂, wherein two instances of R^{a} are joined with the nitrogen atom to which they are attached to form heterocyclyl substituted at least with halogen (e.g., F), OH, —O (substituted or unsubstituted alkyl), or substituted or unsubstituted alkyl (e.g., alkyl substituted at least with —OH, substituted or unsubstituted amino), or substituted or unsubstituted acyl.

In certain embodiments, R₁ is —N(R^{a})₂, wherein two instances of R^{a} are joined with the nitrogen atom to which they are attached to form substituted or unsubstituted, fused bicyclic heterocyclyl. In certain embodiments, the substituted or unsubstituted, fused bicyclic heterocyclyl is substituted or unsubstituted, monocyclic heterocyclyl fused with another substituted or unsubstituted, monocyclic heterocyclyl. In certain embodiments, the substituted or unsubstituted, fused bicyclic heterocyclyl is substituted or unsubstituted, monocyclic heterocyclyl fused with substituted or unsubstituted, monocyclic carbocyclyl. In certain embodiments, the substituted or unsubstituted, fused bicyclic heterocyclyl comprises only carbon and nitrogen atoms in the bicyclic heterocyclic ring system. In certain embodiments, the substituted or unsubstituted, fused bicyclic heterocyclyl comprises only carbon and oxygen atoms in the bicyclic heterocyclic ring system. In certain embodiments, the substituted or unsubstituted, fused bicyclic heterocyclyl comprises only carbon, nitrogen, and oxygen atoms in the bicyclic heterocyclic ring system. In certain embodiments, the fused bicyclic heterocyclyl is substituted at least with halogen (e.g., F), —OH, —O (substituted or unsubstituted alkyl), substituted or unsubstituted alkyl, substituted or unsubstituted acyl.

In certain embodiments, R₁ is —N(R^{a})₂, wherein two instances of R^{a} are joined with the nitrogen atom to which they are attached to form substituted or unsubstituted, bridged bicyclic heterocyclyl. In certain embodiments, the substituted or unsubstituted, bridged bicyclic heterocyclyl is substituted or unsubstituted, monocyclic heterocyclyl bridged with another substituted or unsubstituted, monocyclic heterocyclyl. In certain embodiments, the substituted or unsubstituted, bridged bicyclic heterocyclyl is substituted or unsubstituted, monocyclic heterocyclyl bridged with substituted or unsubstituted, monocyclic carbocyclyl. In certain embodiments, the substituted or unsubstituted, bridged bicyclic heterocyclyl comprises only carbon and nitrogen atoms in the bicyclic heterocyclic ring system. In certain embodiments, the substituted or unsubstituted, bridged bicyclic heterocyclyl comprises only carbon and oxygen atoms in the bicyclic heterocyclic ring system. In certain embodiments, the substituted or unsubstituted, bridged bicyclic heterocyclyl comprises only carbon, nitrogen, and oxygen atoms in the bicyclic heterocyclic ring system. In certain embodiments, the bridged bicyclic heterocyclyl is substituted at least with halogen (e.g., F), —OH, —O (substituted or unsubstituted alkyl), substituted or unsubstituted alkyl, substituted or unsubstituted acyl.

In certain embodiments, R₁ is —N(R^{a})₂, wherein two instances of R^{a} are joined with the nitrogen atom to which they are attached to form substituted or unsubstituted, spiro bicyclic heterocyclyl. In certain embodiments, the substituted or unsubstituted, spiro bicyclic heterocyclyl is substituted or unsubstituted, monocyclic heterocyclyl spiroed with another substituted or unsubstituted, monocyclic heterocyclyl. In certain embodiments, the substituted or unsubstituted, spiro bicyclic heterocyclyl is substituted or unsubstituted, monocyclic heterocyclyl spiroed with substituted or unsubstituted, monocyclic carbocyclyl. In certain embodiments, the substituted or unsubstituted, spiro bicyclic heterocyclyl comprises only carbon and nitrogen atoms in the bicyclic heterocyclic ring system. In certain embodiments, the substituted or unsubstituted, spiro bicyclic heterocyclyl comprises only carbon and oxygen atoms in the bicyclic heterocyclic ring system. In certain embodiments, the substituted or unsubstituted, spiro bicyclic heterocyclyl comprises only carbon, nitrogen, and oxygen atoms in the bicyclic heterocyclic ring system. In certain embodiments, the spiro bicyclic heterocyclyl is substituted at least with halogen (e.g., F), —OH, —O (substituted or unsubstituted alkyl), substituted or unsubstituted alkyl, substituted or unsubstituted acyl.

In certain embodiments, R₁ is

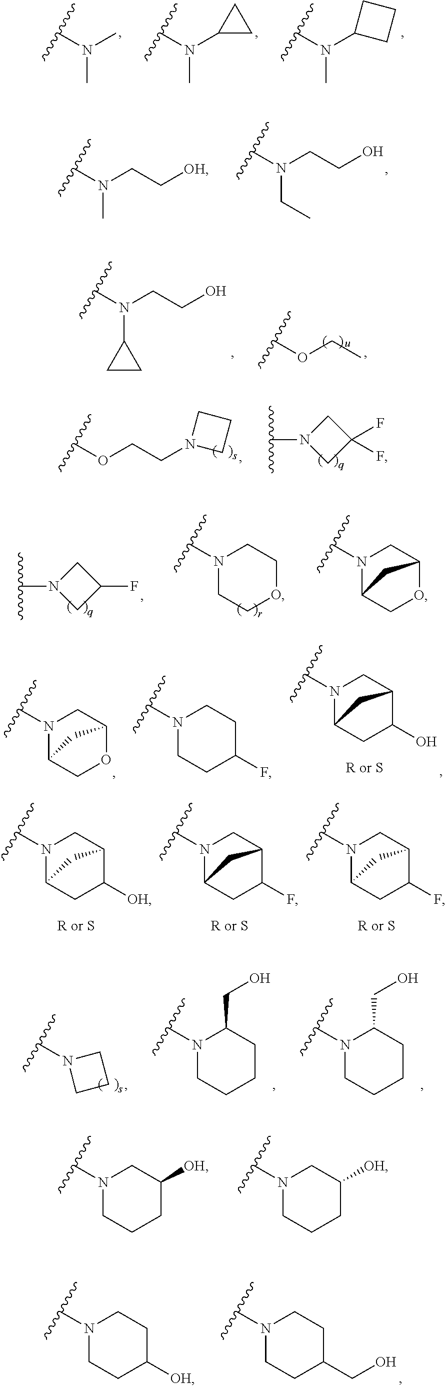

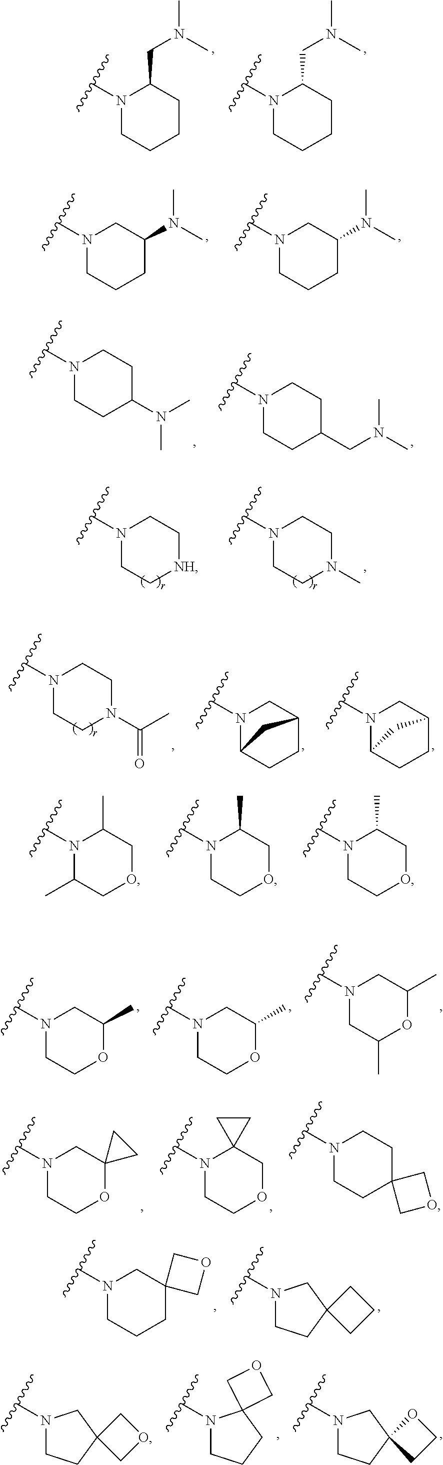

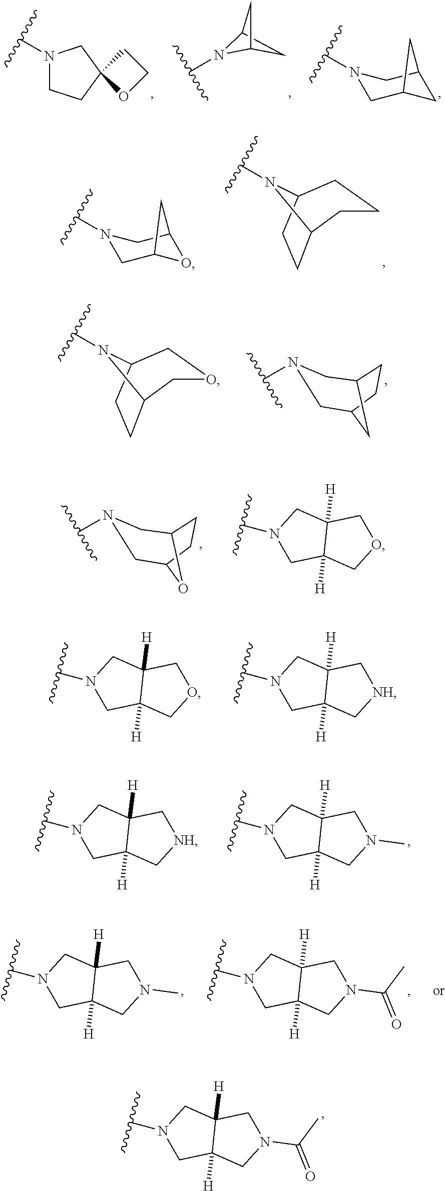

wherein:
   each n is independently 0, 1, 2, 3, 4, 5, 6, 7, 8, 9, or 10;
   each p is independently 0, 1, 2, 3, or 4;
   each q is independently 1, 2, or 3;
   each r is independently 1 or 2;
   each s is independently 1, 2, 3, or 4;
   each t is independently 1, 2, 3, 4, or 5; and
   each u is independently 0, 1, 2, 3, 4, or 5.

In certain embodiments, R₁ is of the formula:

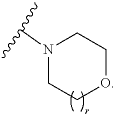

In certain embodiments, R₁ is of the formula:

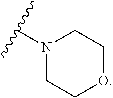 [SMILES: *N1CCOCC1]

In certain embodiments, R₁ is of the formula:

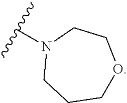 [SMILES: *N1CCCOCC1]

In certain embodiments, R₂ is NO₂. In certain embodiments, R₂ is SO₂CF₃.

In certain embodiments, R₃ is

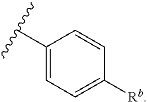

In certain embodiments, R₃ is

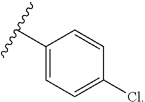 [SMILES: *c1ccc(Cl)cc1]

In certain embodiments, R³ is

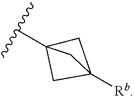

In certain embodiments, R^{b} is halogen. In certain embodiments, R^{b} is Cl. In certain embodiments, R^{b} is F. In certain embodiments, R^{b} is substituted or unsubstituted alkyl. In certain embodiments, R^{b} is unsubstituted C₁₋₆ alkyl. In certain embodiments, R^{b} is —CH₃. In certain embodiments, R^{b} is substituted C₁₋₆ alkyl. In certain embodiments, R^{b} is substituted methyl. In certain embodiments, R^{b} is methyl substituted at least with halogen. In certain embodiments, R^{b} is alkyl substituted only with one or more (e.g., two or three) instances of fluoro. In certain embodiments, R^{b} is —CFH₂, —CF₂H, or —CF₃.

In certain embodiments, R₃ is

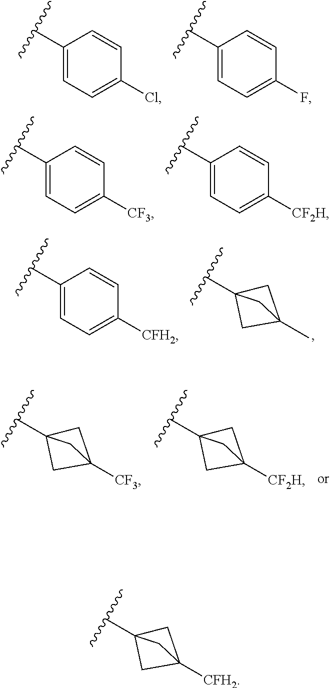

In certain embodiments, Z is a bond. In certain embodiments, Z is CH₂. In certain embodiments, Z is CO.

In certain embodiments, Y is CO.

In certain embodiments,

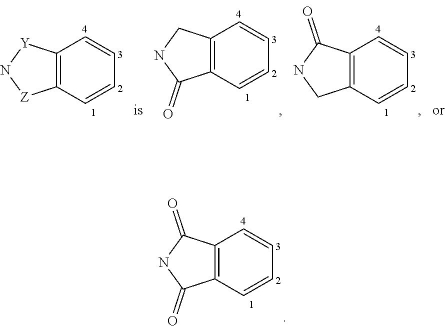

In certain embodiments,

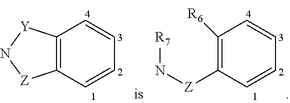

In certain embodiments,

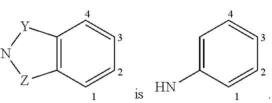

In certain embodiments,

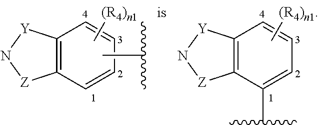

In certain embodiments,

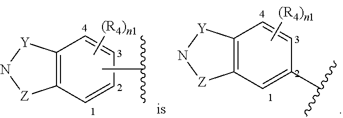

In certain embodiments,

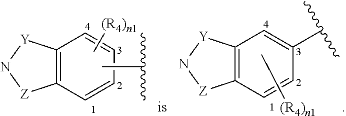

In certain embodiments,

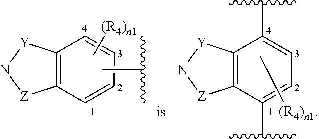

In certain embodiments, n1 is 0. In certain embodiments, n1 is 1. In certain embodiments, n1 is 1, 2, or 3, and each instance of R₄ is independently F, Cl, —CN, —CFH₂, —CF₂H, —CF₃, or —OCH₃. In certain embodiments, at least one instance of R₄ is F.

In certain embodiments, L₁ is substituted or unsubstituted, C₁₋₁₁ alkylene. In certain embodiments, L₁ is unsubstituted C₁₋₂ alkylene. In certain embodiments, L₁ is —CH₂—. In certain embodiments, L₁ is unsubstituted, C₃₋₅ alkylene. In certain embodiments, L₁ is unsubstituted C₆₋₈ alkylene. In certain embodiments, L₁ is unsubstituted, C₉₋₁₁ alkylene.

In certain embodiments, L₁ is substituted or unsubstituted, C₁₋₁₁ alkylene, wherein one or more (e.g., two or three) backbone carbon atoms in the substituted or unsubstituted, C₁₋₁₁ alkylene are independently replaced with —O—, —NR₅—, —S(═O)—, —S(═O)₂—, or —C(═O)—. In certain embodiments, L₁ is substituted or unsubstituted, C₁₋₃ alkylene, wherein one or more (e.g., two or three) backbone carbon atoms in the substituted or unsubstituted, C₁₋₃ alkylene are independently replaced with —O—, —NR₅—, —S(═O)—, —S(═O)₂—, or —C(═O)—. In certain embodiments, L₁ is substituted or unsubstituted, C₄₋₅ alkylene, wherein one or more (e.g., two or three) backbone carbon atoms in the substituted or unsubstituted, C₄₋₅ alkylene are independently replaced with —O—, —NR₅—, —S(═O)—, —S(═O)₂—, or —C(═O)—. In certain embodiments, L₁ is substituted or unsubstituted, C₆₋₇ alkylene, wherein one or more (e.g., two or three) backbone carbon atoms in the substituted or unsubstituted, C₆₋₇ alkylene are independently replaced with —O—, —NR₅—, —S(═O)—, —S(═O)₂—, or —C(═O)—. In certain embodiments, L₁ is substituted or unsubstituted, C₈₋₉ alkylene, wherein one or more (e.g., two or three) backbone carbon atoms in the substituted or unsubstituted, C₈₋₉ alkylene are independently replaced with —O—, —NR₅—, —S(═O)—, —S(═O)₂—, or —C(═O)—. In certain embodiments, L₁ is substituted or unsubstituted, C₁₀₋₁₁ alkylene, wherein one or more (e.g., two or three) backbone carbon atoms in the substituted or unsubstituted, C₁₀₋₁₁ alkylene are independently replaced with —O—, —NR₅—, —S(═O)—, —S(═O)₂—, or —C(═O)—. In certain embodiments, L₁ is substituted or unsubstituted, C₂ alkylene, wherein one or two backbone carbon atoms in the substituted or unsubstituted, C₂ alkylene are independently replaced with —O—, —NR₅—, —S(═O)—, —S(═O)₂—, or —C(═O)—. In certain embodiments, L₁ is substituted or unsubstituted, C₃ alkylene, wherein one, two, or three backbone carbon atoms in the substituted or unsubstituted, C₃ alkylene are independently replaced with —O—, —NR₅—, —S(═O)—, —S(═O)₂—, or —C(═O)—.

In certain embodiments, L₁ is substituted or unsubstituted, monocyclic heterocyclylene, substituted or unsubstituted, bicyclic heterocyclylene, or substituted or unsubstituted, monocyclic heteroarylene.

In certain embodiments, L₁ is -(substituted or unsubstituted, monocyclic heterocyclylene)-(substituted or unsubstituted, C₁₋₂ alkylene)-, -(substituted or unsubstituted, bicyclic heterocyclylene)-(substituted or unsubstituted, C₁₋₂ alkylene)-, -(substituted or unsubstituted, monocyclic heteroarylene)-(substituted or unsubstituted, C₁₋₂ alkylene)-, —(substituted or unsubstituted, C₁₋₂ alkylene)-(substituted or unsubstituted, monocyclic heterocyclylene)-, -(substituted or unsubstituted, C₁₋₂ alkylene)-(substituted or unsubstituted, bicyclic heterocyclylene)-, or (substituted or unsubstituted, C₁₋₂ alkylene)-(substituted or unsubstituted, monocyclic heteroarylene)-, optionally wherein one or two backbone carbon atoms in the substituted or unsubstituted, C₁₋₂ alkylene are independently replaced with —O—, —NR₅—, —S(═O)—, —S(═O)₂—, or —C(═O)—.

In certain embodiments, L₁ is -(substituted or unsubstituted, C₁₋₂ alkylene)-(substituted or unsubstituted, monocyclic heterocyclylene)-(substituted or unsubstituted, C₁₋₂ alkylene)-, —(substituted or unsubstituted, C₁₋₂ alkylene)-(substituted or unsubstituted, bicyclic heterocyclylene)-(substituted or unsubstituted, C₁₋₂ alkylene)-, or -(substituted or unsubstituted, C₁₋₂ alkylene)-(substituted or unsubstituted, monocyclic heteroarylene)-(substituted or unsubstituted, C₁₋₂ alkylene)-, optionally wherein one or two backbone carbon atoms in each instance of the substituted or unsubstituted, C₁₋₂ alkylene are independently replaced with —O—, —NR₅—, —S(═O)—, —S(═O)₂—, or —C(═O)—.

In certain embodiments, L₁ is -(substituted or unsubstituted, monocyclic heterocyclylene)₂-(substituted or unsubstituted, C₁ alkylene)-, -(substituted or unsubstituted, C₁ alkylene)-(substituted or unsubstituted, monocyclic heterocyclylene)₂-, -(substituted or unsubstituted, C₁ alkylene)-(substituted or unsubstituted, monocyclic heterocyclylene)₂-(substituted or unsubstituted, C₁ alkylene)-, -(substituted or unsubstituted, monocyclic heterocyclylene)-(substituted or unsubstituted, monocyclic carbocyclylene)-(substituted or unsubstituted, C₁ alkylene)-, -(substituted or unsubstituted, monocyclic carbocyclylene)-(substituted or unsubstituted, monocyclic heterocyclylene)-(substituted or unsubstituted, C₁ alkylene)-, -(substituted or unsubstituted, C₁ alkylene)-(substituted or unsubstituted, monocyclic heterocyclylene)-(substituted or unsubstituted, monocyclic carbocyclylene)-, or -(substituted or unsubstituted, C₁ alkylene)-(substituted or unsubstituted, monocyclic carbocyclylene)-(substituted or unsubstituted, monocyclic heterocyclylene)-, optionally wherein the backbone carbon atom in each instance of the substituted or unsubstituted, C₁ alkylene is independently replaced with —O—, —NR₅—, —S(═O)—, —S(═O)₂—, or —C(═O)—.

In certain embodiments, L₁ is

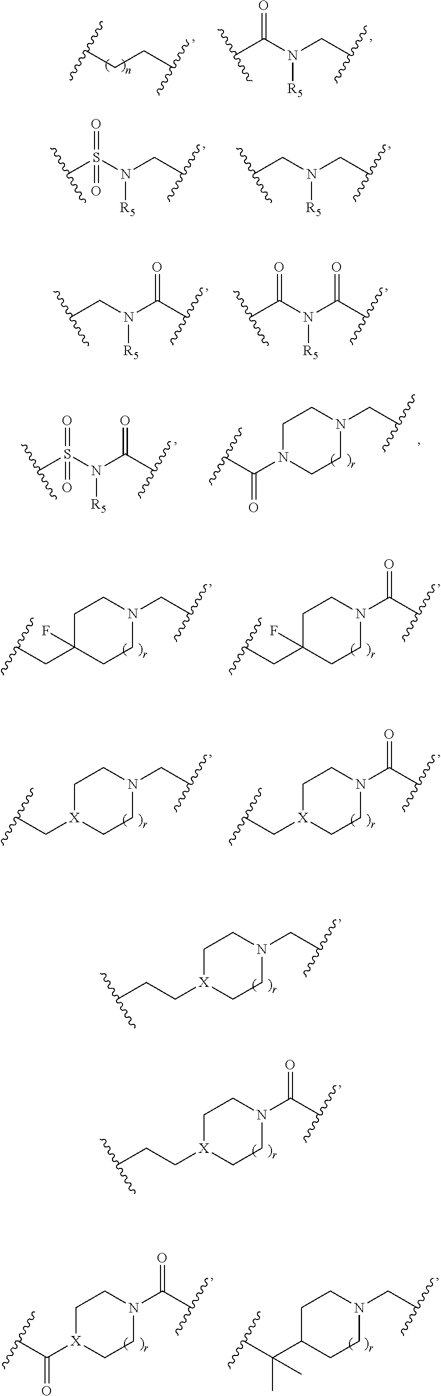

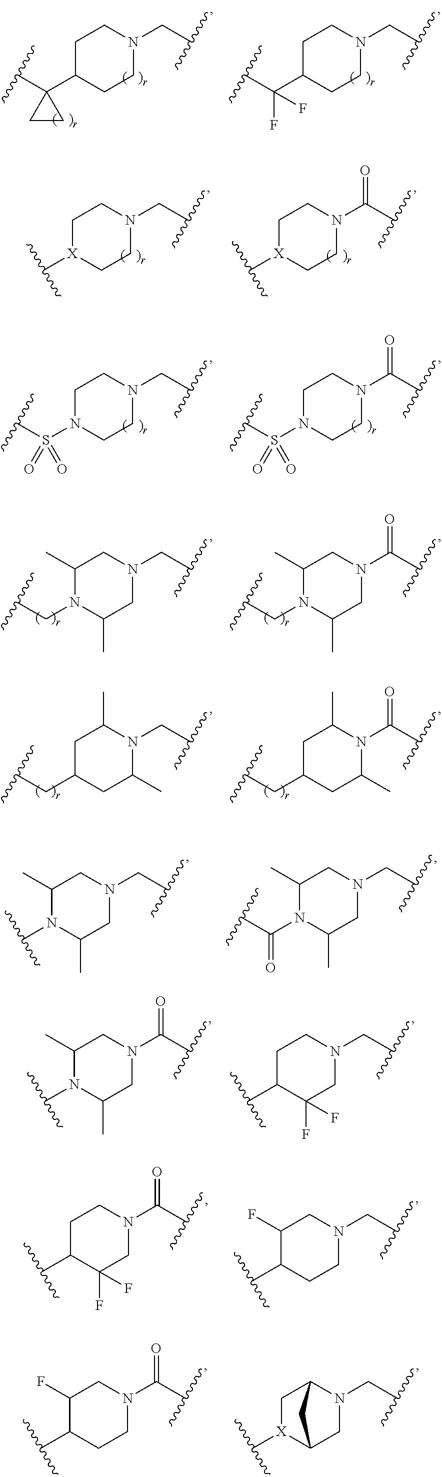

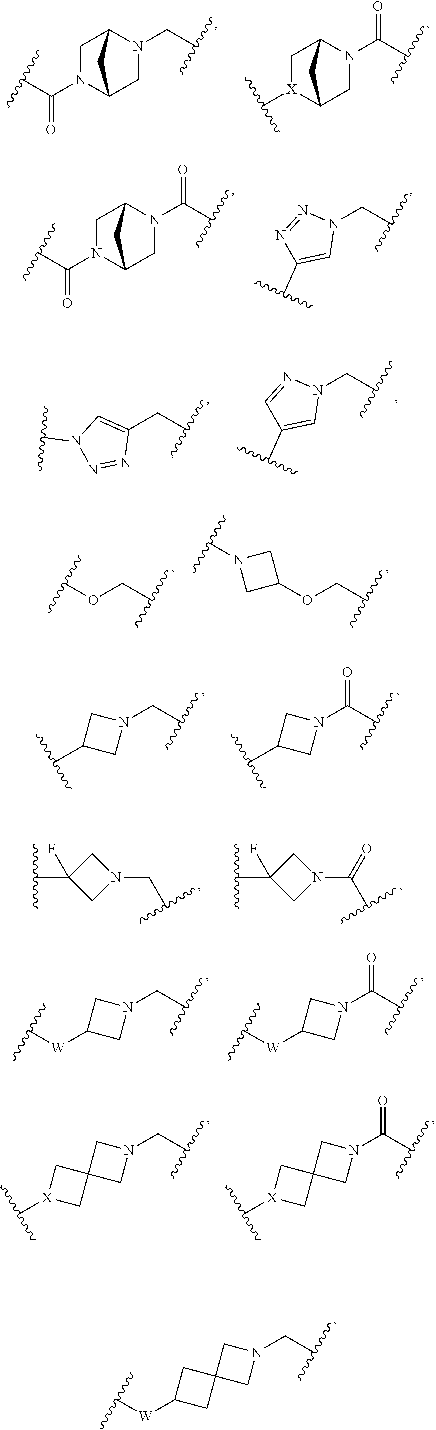

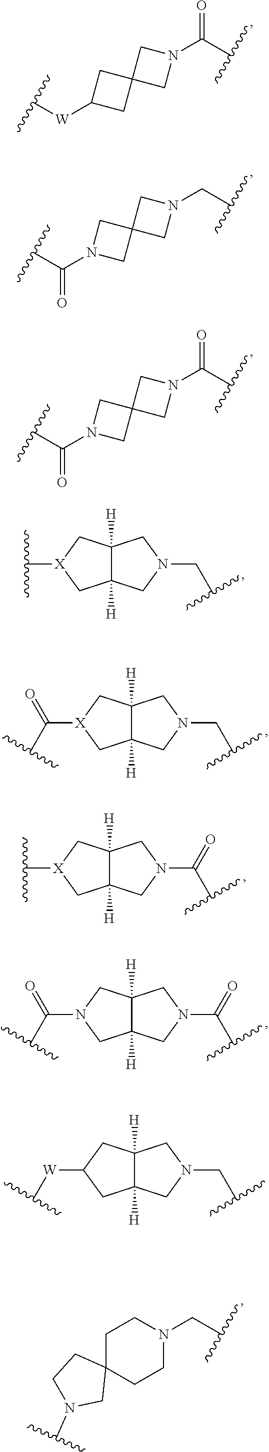

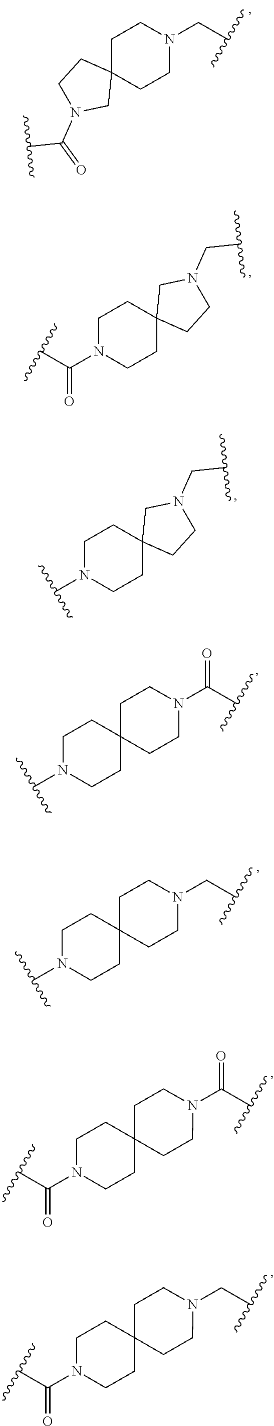

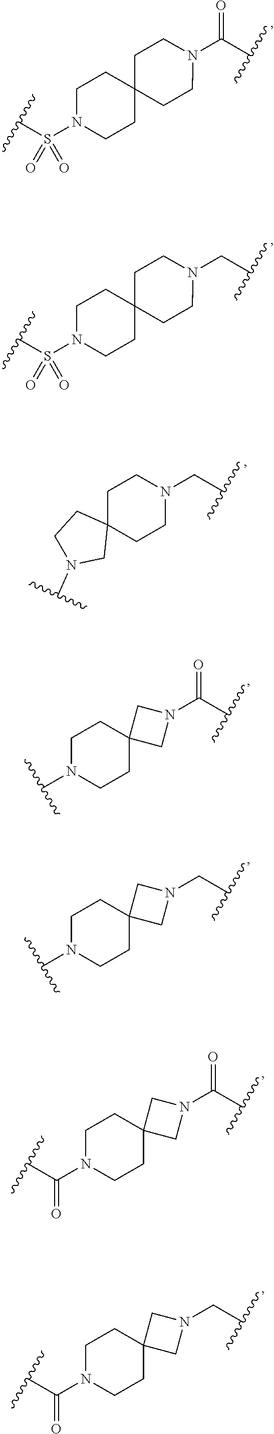

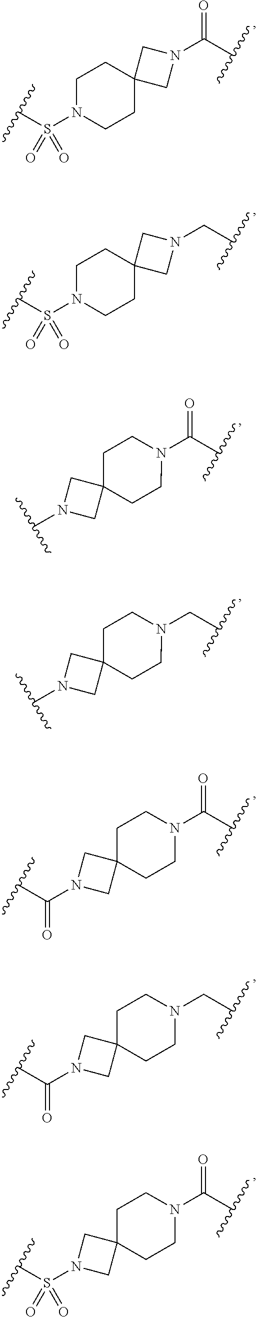

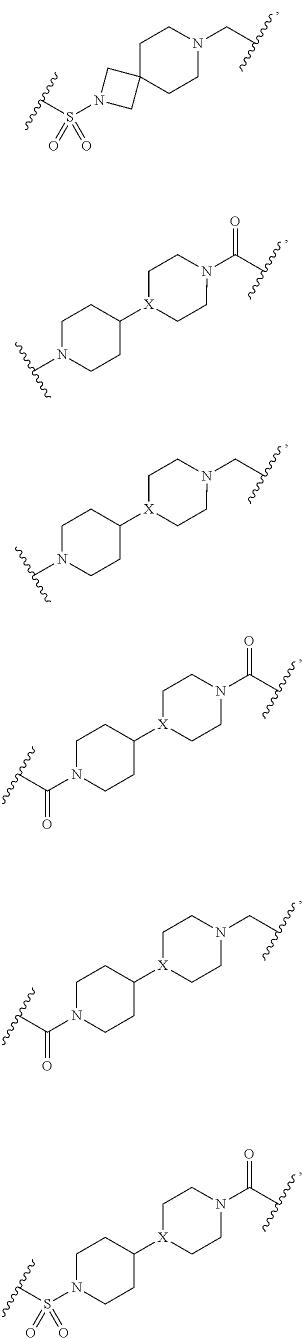

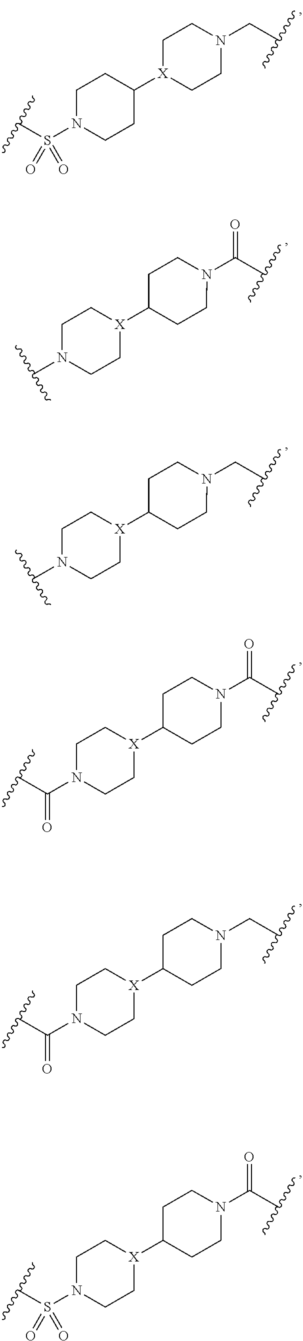

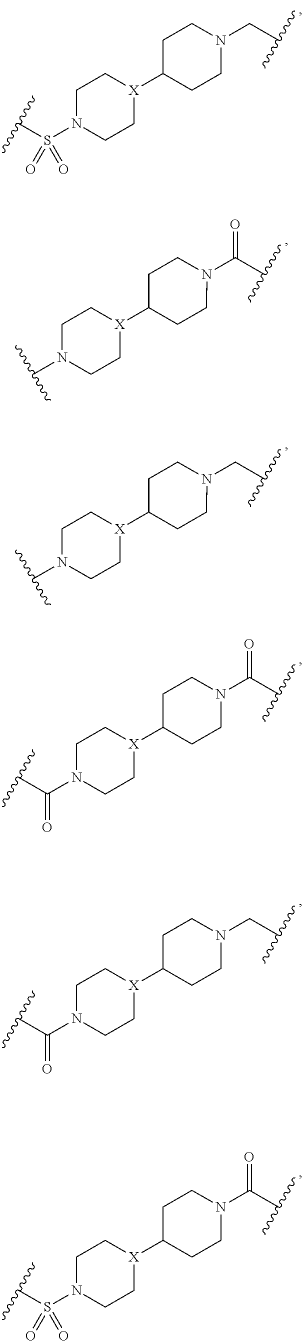

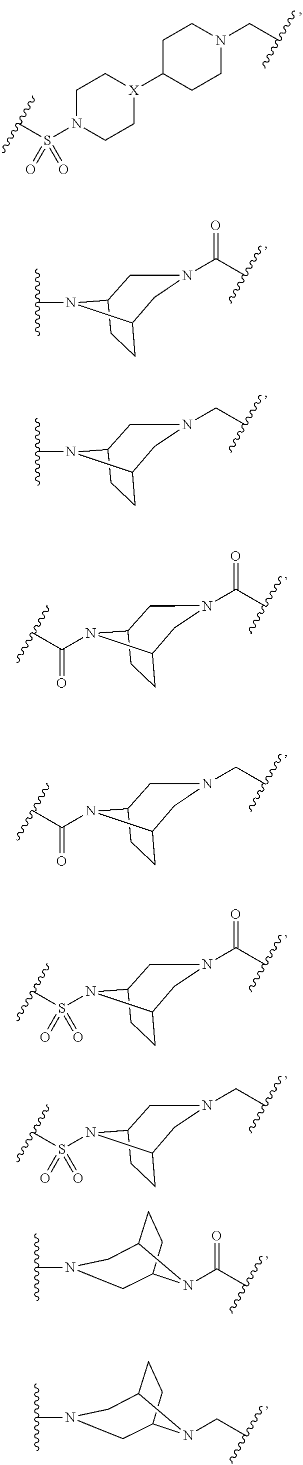

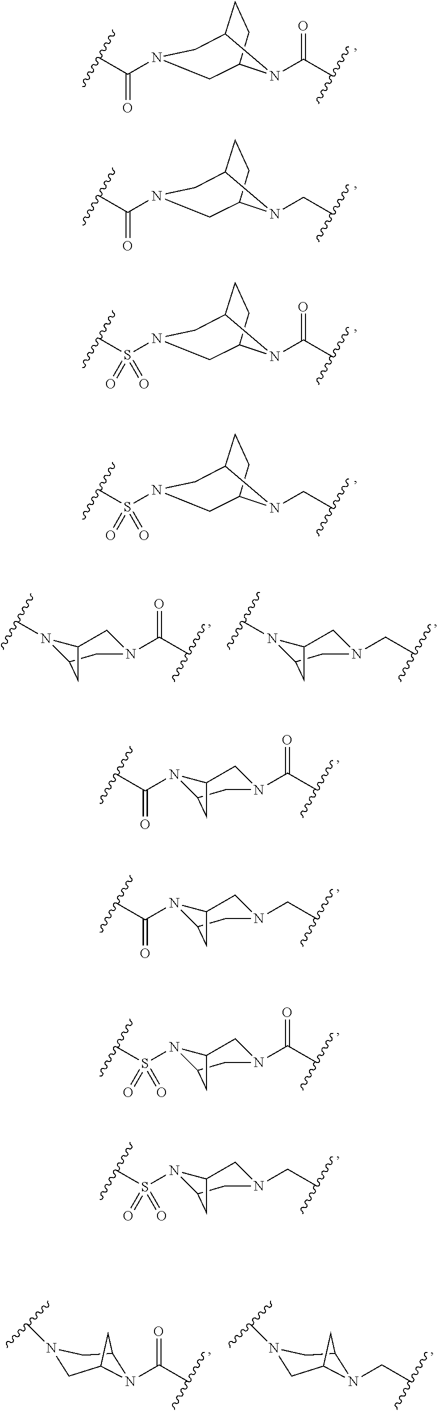

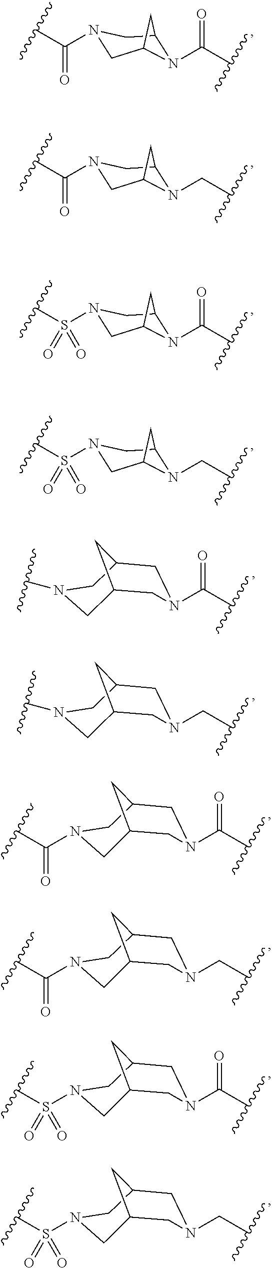

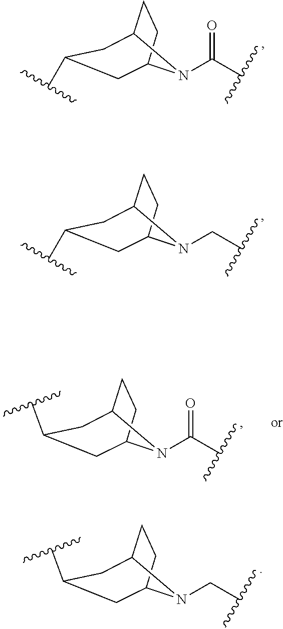 [SMILES: *C(=O)N1C2CCC1CC(*)C2 ; *CN1C2CCC1CC(*)C2 ; *C(=O)N1C2CCC1CC(*)C2 ; *CN1C2CCC1CC(*)C2]

Each n is independently 0, 1, 2, 3, 4, 5, 6, 7, 8, 9, or 10.

Each p is independently 0, 1, 2, 3, or 4.

Each q is independently 1, 2, or 3.

Each r is independently 1 or 2.

Each s is independently 1, 2, 3, or 4.

Each t is independently 1, 2, 3, 4, or 5.

Each u is independently 0, 1, 2, 3, 4, or 5.

In certain embodiments, L₁ is

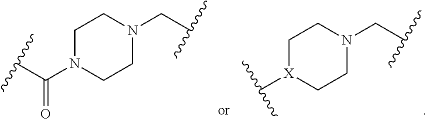

In certain embodiments, at least one instance of R₅ is hydrogen. In certain embodiments, at least one instance of R₅ is substituted or unsubstituted alkyl or substituted or unsubstituted carbocyclyl. In certain embodiments, each instance of R₅ is hydrogen. In certain embodiments, at least one instance of R₅ is not hydrogen. In certain embodiments, no instance of R₅ is hydrogen. In certain embodiments, at least one instance of R₅ is substituted alkyl (e.g., alkyl substituted with one or more (e.g., two or three) instances of halogen (e.g., F)). In certain embodiments, at least one instance of R₅ is unsubstituted alkyl. In certain embodiments, at least one instance of R₅ is unsubstituted C₁₋₆ alkyl. In certain embodiments, at least one instance of R₅ is Me. In certain embodiments, at least one instance of R₅ is Et, Pr, or Bu. In certain embodiments, at least one instance of R₅ is substituted C₁₋₆ alkyl. In certain embodiments, at least one instance of R₅ is substituted methyl (e.g., fluorinated methyl or Bn). In certain embodiments, at least one instance of R₅ is substituted ethyl, substituted propyl, or substituted butyl. In certain embodiments, at least one instance of R₅ is substituted or unsubstituted alkenyl. In certain embodiments, at least one instance of R₅ is substituted or unsubstituted, C₂₋₆ alkenyl (e.g., substituted or unsubstituted vinyl or substituted or unsubstituted allyl). In certain embodiments, at least one instance of R₅ is substituted or unsubstituted alkynyl. In certain embodiments, at least one instance of R₅ is substituted or unsubstituted, C₂₋₆ alkynyl (e.g., substituted or unsubstituted ethynyl). In certain embodiments, at least one instance of R₅ is substituted or unsubstituted carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R₅ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R₅ is substituted or unsubstituted heterocyclyl (e.g., substituted or unsubstituted, 3- to 7-membered, monocyclic heterocyclyl). In certain embodiments, at least one instance of R₅ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R₅ is substituted or unsubstituted aryl. In certain embodiments, at least one instance of R₅ is substituted or unsubstituted phenyl. In certain embodiments, at least one instance of R₅ is substituted or unsubstituted naphthyl. In certain embodiments, at least one instance of R₅ is substituted or unsubstituted heteroaryl. In certain embodiments, at least one instance of R₅ is substituted or unsubstituted, 5- to 6-membered, monocyclic heteroaryl. In certain embodiments, at least one instance of R₅ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R₅ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R₅ is substituted or unsubstituted, 9- to 10-membered, bicyclic heteroaryl.

In certain embodiments, L₂ is substituted or unsubstituted, C₁₋₁₂ alkylene, substituted or unsubstituted, C₂₋₁₂ alkenylene, or substituted or unsubstituted, C₂₋₁₂ alkynylene. In certain embodiments, L₂ is unsubstituted, C₁₋₂ alkylene. In certain embodiments, L₂ is unsubstituted, C₃₋₄ alkylene. In certain embodiments, L₂ is unsubstituted, C₅₋₆ alkylene. In certain embodiments, L₂ is unsubstituted, C₇₋₉ alkylene. In certain embodiments, L₂ is unsubstituted, C₁₀₋₁₂ alkylene. In certain embodiments, L₂ is unsubstituted C₂₋₁₂ alkenylene comprising only one unsaturated CC bond in the backbone. In certain embodiments, L₂ is unsubstituted C₂₋₁₂ alkynylene comprising only one unsaturated CC bond in the backbone.

In certain embodiments, L₂ is substituted or unsubstituted, C₁₋₁₂ alkylene, substituted or unsubstituted, C₂₋₁₂ alkenylene, or substituted or unsubstituted, C₂₋₁₂ alkynylene, wherein one or more (e.g., two or three) backbone carbon atoms in the substituted or unsubstituted, C₁₋₁₂ alkylene, substituted or unsubstituted, C₂₋₁₂ alkenylene, or substituted or unsubstituted, C₂₋₁₂ alkynylene are independently replaced with —O—, —NR₅—, —S(═O)—, —S(═O)₂—, or —C(═O)—.

In certain embodiments, L₂ is -(substituted or unsubstituted, monocyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, bicyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, monocyclic heteroarylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, monocyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, —(substituted or unsubstituted, bicyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heterocyclylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic heterocyclylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heteroarylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic carbocyclylene)-, or -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic carbocyclylene)-, optionally wherein one or more (e.g., two or three) backbone carbon atoms in the substituted or unsubstituted, C₁₋₈ alkylene, substituted or unsubstituted, C₂₋₈ alkenylene, or substituted or unsubstituted, C₂₋₈ alkynylene are independently replaced with —O—, —NR₅—, —S(═O)—, —S(═O)₂—, or —C(═O)—.

In certain embodiments, L₂ is -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, —(substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heteroarylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, or -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, optionally wherein one or more (e.g., two or three) backbone carbon atoms in each instance of the substituted or unsubstituted, C₁₋₈ alkylene, substituted or unsubstituted, C₂₋₈ alkenylene, or substituted or unsubstituted, C₂₋₈ alkynylene are independently replaced with —O—, —NR₅—, —S(═O)—, —S(═O)₂—, or —C(═O)—.

In certain embodiments, L₂ is -(substituted or unsubstituted, monocyclic heterocyclylene)₂-(substituted or unsubstituted, C₁₋₂ alkylene)-, -(substituted or unsubstituted, C₁₋₂ alkylene)-(substituted or unsubstituted, monocyclic heterocyclylene)₂-, or -(substituted or unsubstituted, C₁₋₂ alkylene)-(substituted or unsubstituted, monocyclic heterocyclylene)₂-(substituted or unsubstituted, C₁₋₂ alkylene)-, optionally wherein one or more (e.g., two or three) of the backbone carbon atoms in each instance of the substituted or unsubstituted, C₁₋₂ alkylene are independently replaced with —O—, —NR₅—, —S(═O)—, —S(═O)₂—, or —C(═O)—.

In certain embodiments, L₂ is substituted or unsubstituted heterocyclylene. In certain embodiments, L₂ is substituted or unsubstituted, monocyclic heterocyclylene. In certain embodiments, L₂ is substituted or unsubstituted, monocyclic, 3- to 8-membered heterocyclylene. In certain embodiments, L₂ is substituted or unsubstituted, bicyclic heterocyclylene. In certain embodiments, L₂ is substituted or unsubstituted, bicyclic, 5- to 14-membered heterocyclylene.

In certain embodiments, L₂ is

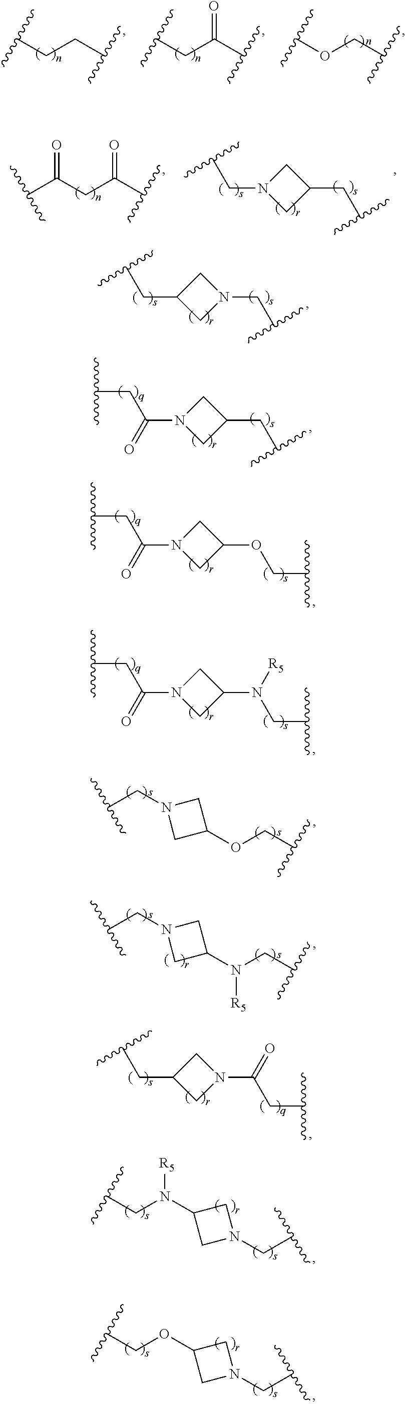

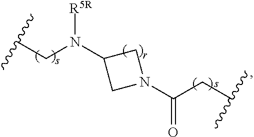

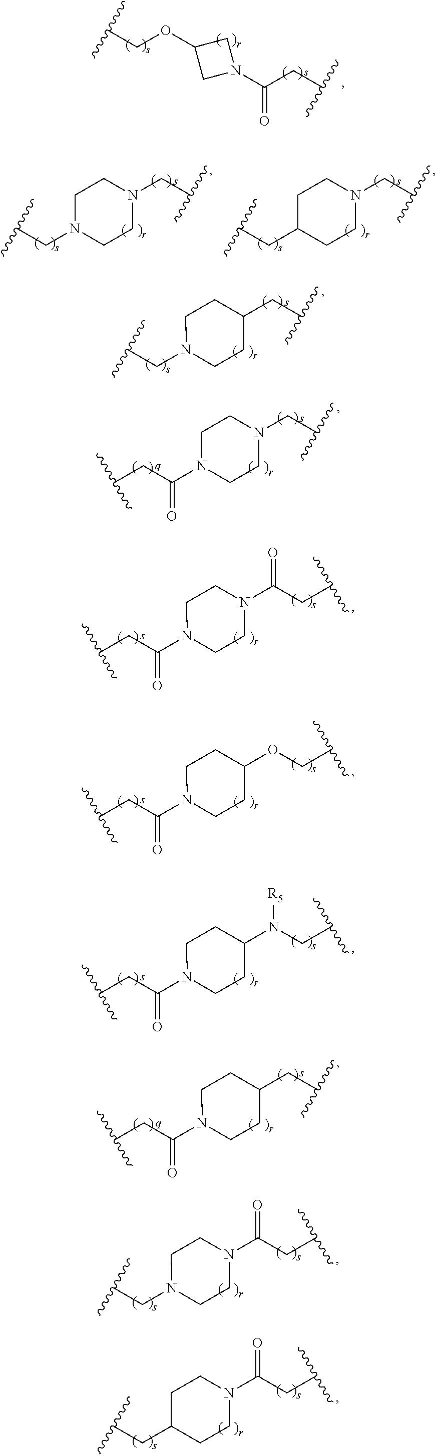

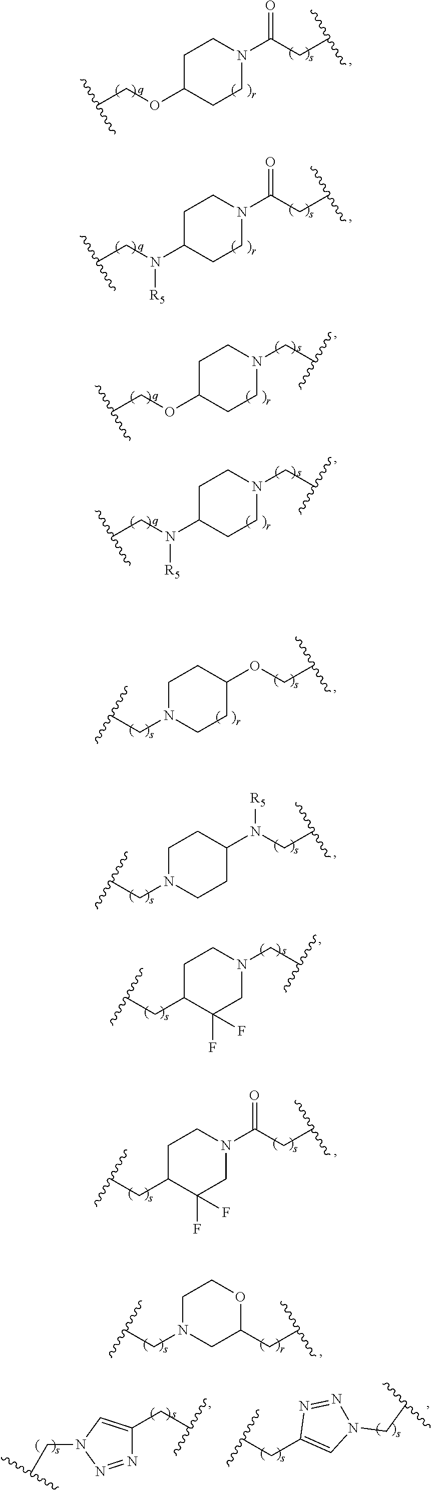

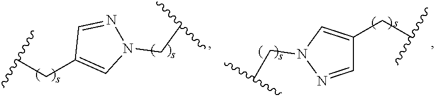

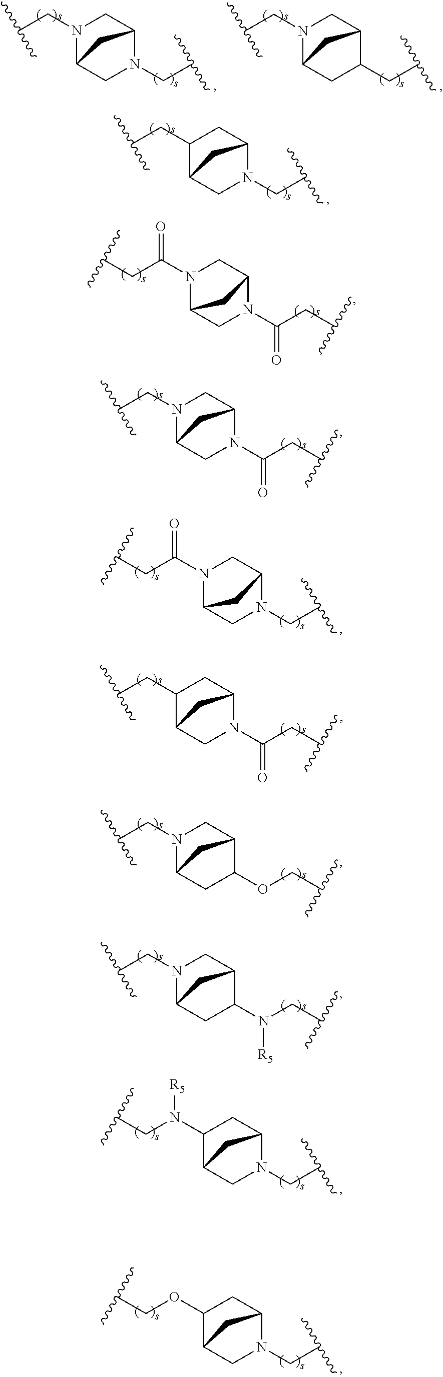

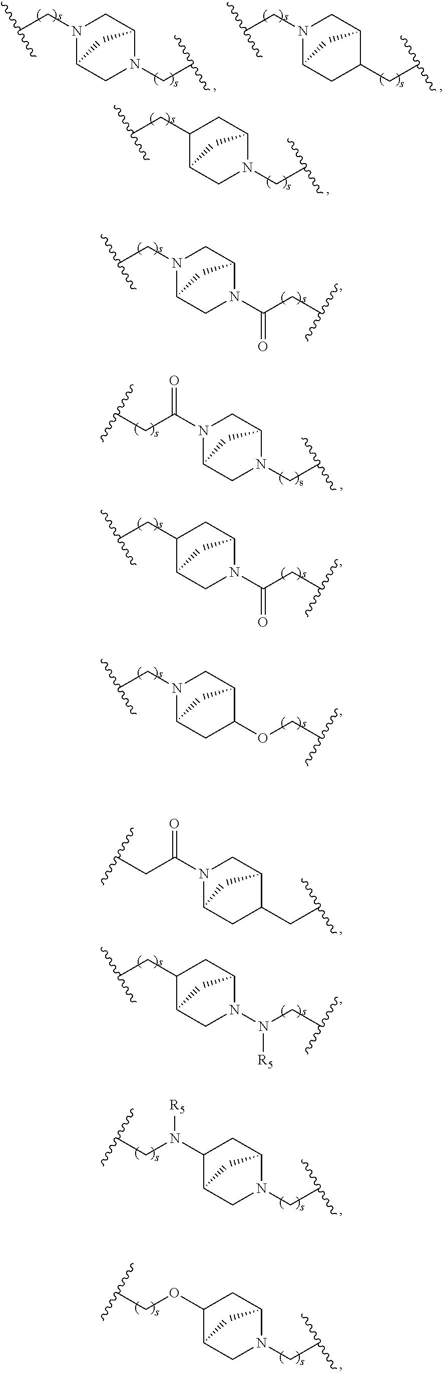

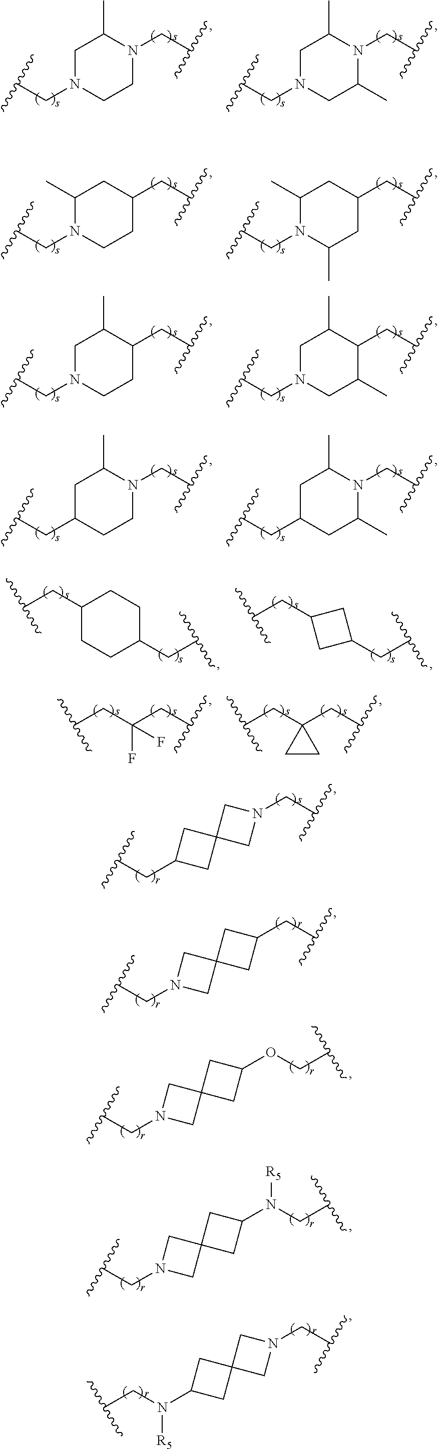

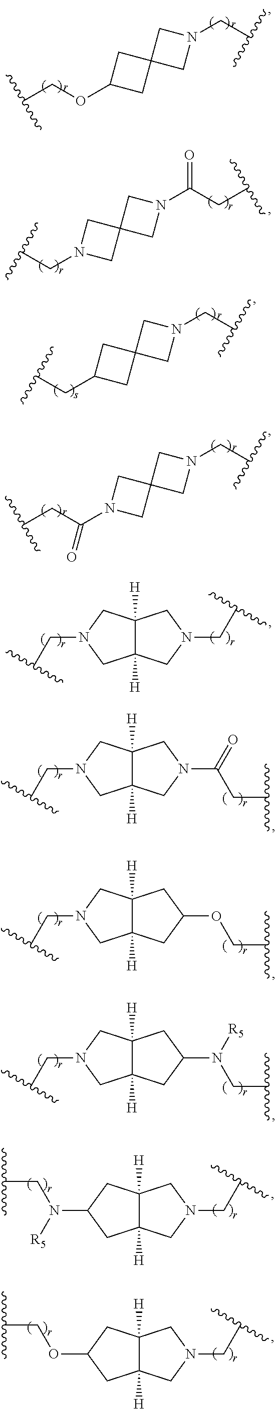

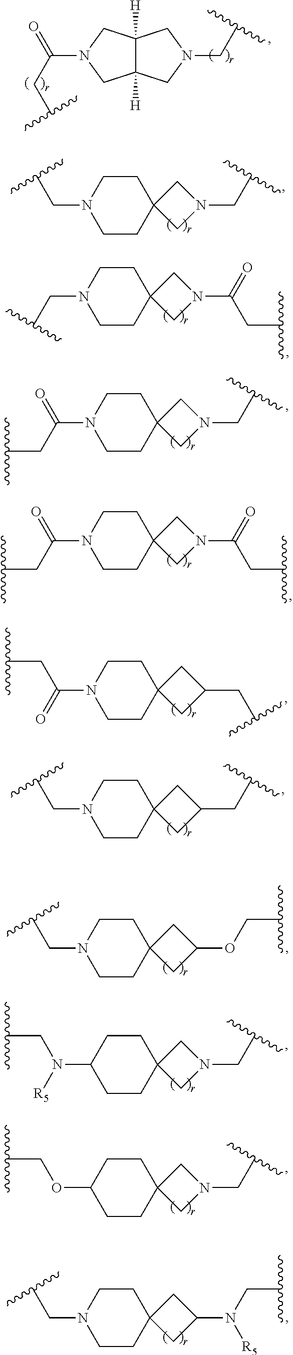

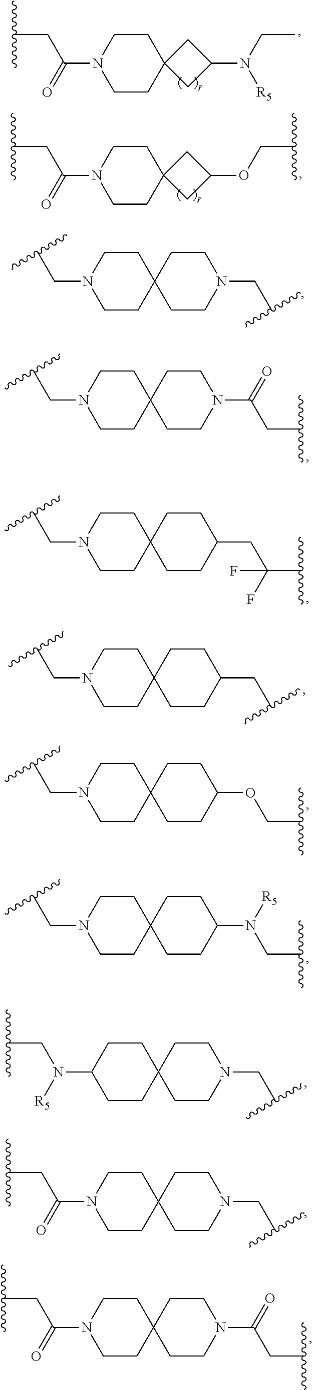

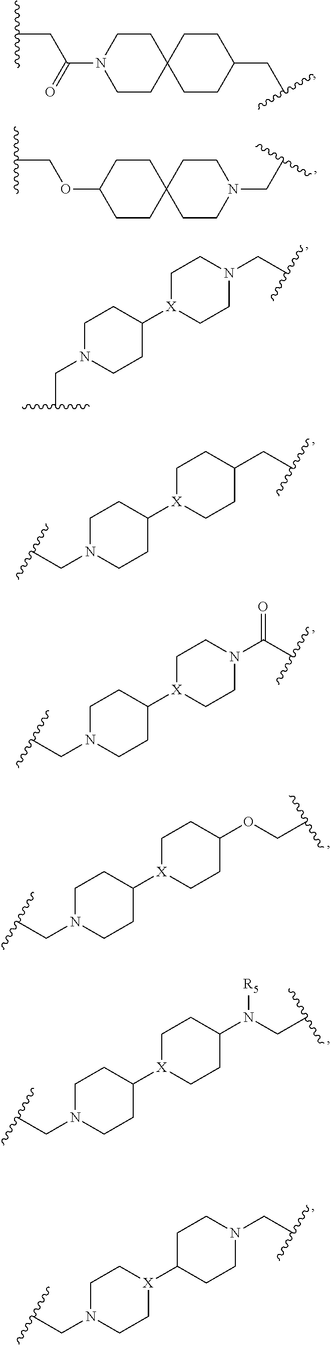

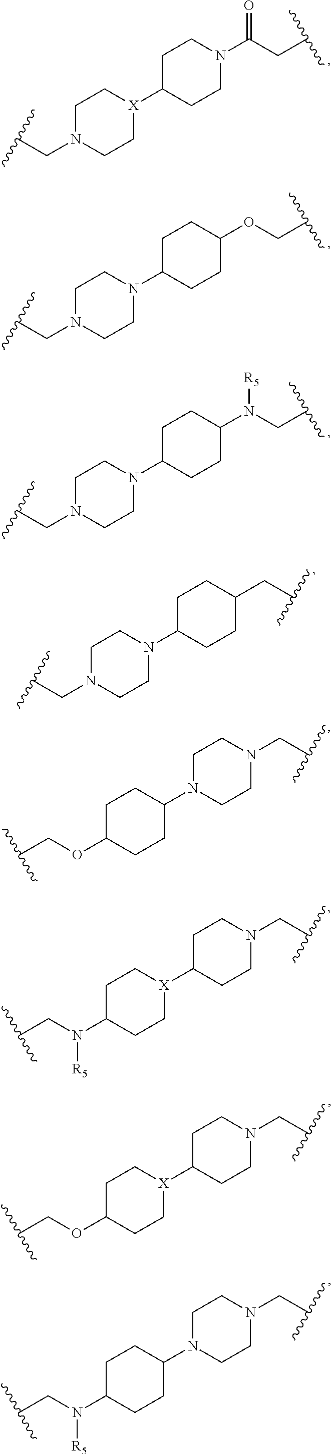

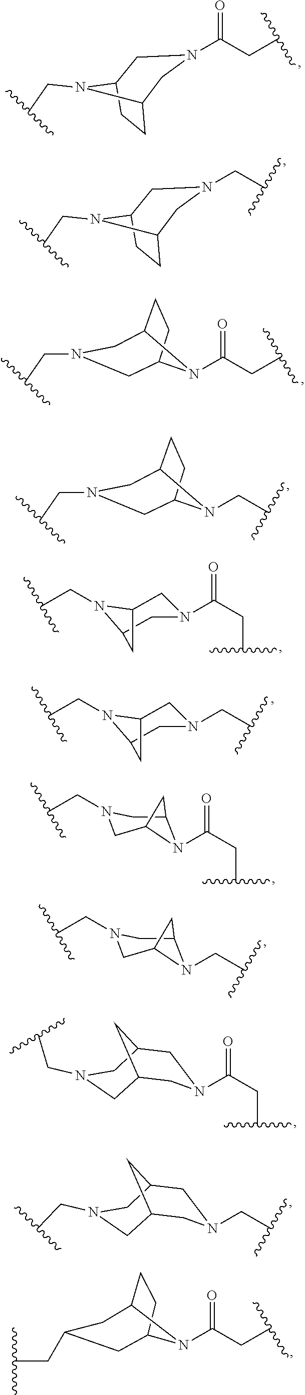

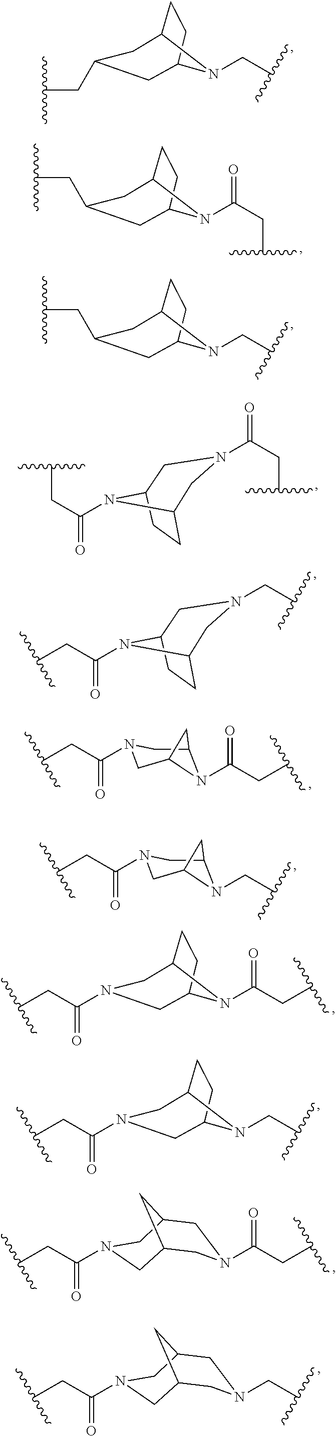

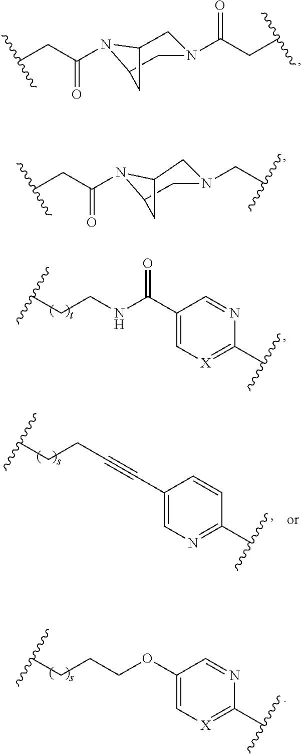

In certain embodiments, R₆ is F, Cl, —CN, —CFH₂, —CF₂H, —CF₃, or —OCH₃. In certain embodiments, R₆ is H.

In certain embodiments, L₃ is a bond. In certain embodiments, L₃ is substituted or unsubstituted, C₁₋₁₃ alkylene, substituted or unsubstituted, C₂₋₁₃ alkenylene, or substituted or unsubstituted, C₂₋₁₃ alkynylene. In certain embodiments, L₃ is unsubstituted, C₁₋₂ alkylene. In certain embodiments, L₃ is unsubstituted, C₃₋₄ alkylene. In certain embodiments, L₃ is unsubstituted, C₅₋₆ alkylene. In certain embodiments, L₃ is unsubstituted, C₇₋₁₀ alkylene. In certain embodiments, L₃ is unsubstituted, C₁₀₋₁₃ alkylene. In certain embodiments, L₃ is unsubstituted C₂₋₁₃ alkenylene comprising only one unsaturated CC bond in the backbone. In certain embodiments, L₃ is unsubstituted C₂₋₁₃ alkynylene comprising only one unsaturated CC bond in the backbone.

In certain embodiments, L₃ is substituted or unsubstituted, C₁₋₁₃ alkylene, wherein one or more (e.g., two or three) backbone carbon atoms in the substituted or unsubstituted, C₁₋₁₃ alkylene are independently replaced with —O—, —NR₅—, —S(═O)—, —S(═O)₂—, or —C(═O)—. In certain embodiments, L₃ is substituted or unsubstituted, C₁₋₃ alkylene, wherein one or more (e.g., two or three) backbone carbon atoms in the substituted or unsubstituted, C₁₋₃ alkylene are independently replaced with —O—, —NR₅—, —S(═O)—, —S(═O)₂—, or —C(═O)—. In certain embodiments, L₃ is substituted or unsubstituted, C₄₋₅ alkylene, wherein one or more (e.g., two or three) backbone carbon atoms in the substituted or unsubstituted, C₄₋₅ alkylene are independently replaced with —O—, —NR₅—, —S(═O)—, —S(═O)₂—, or —C(═O)—. In certain embodiments, L₃ is substituted or unsubstituted, C₆₋₇ alkylene, wherein one or more (e.g., two or three) backbone carbon atoms in the substituted or unsubstituted, C₆₋₇ alkylene are independently replaced with —O—, —NR₅—, —S(═O)—, —S(═O)₂—, or —C(═O)—. In certain embodiments, L₃ is substituted or unsubstituted, C₈₋₉ alkylene, wherein one or more (e.g., two or three) backbone carbon atoms in the substituted or unsubstituted, C₈₋₉ alkylene are independently replaced with —O—, —NR₅—, —S(═O)—, —S(═O)₂—, or —C(═O)—. In certain embodiments, L₃ is substituted or unsubstituted, C₁₀₋₁₃ alkylene, wherein one or more (e.g., two or three) backbone carbon atoms in the substituted or unsubstituted, C₁₀₋₁₃ alkylene are independently replaced with —O—, —NR₅—, —S(═O)—, —S(═O)₂—, or —C(═O)—. In certain embodiments, L₃ is substituted or unsubstituted, C₂ alkylene, wherein one or two backbone carbon atoms in the substituted or unsubstituted, C₂ alkylene are independently replaced with —O—, —NR₅—, —S(═O)—, —S(═O)₂—, or —C(═O)—. In certain embodiments, L₃ is substituted or unsubstituted, C₃ alkylene, wherein one, two, or three backbone carbon atoms in the substituted or unsubstituted, C₃ alkylene are independently replaced with —O—, —NR₅—, —S(═O)—, —S(═O)₂—, or —C(═O)—. In certain embodiments, L₃ is substituted or unsubstituted, C₄ alkylene, wherein one, two, or three backbone carbon atoms in the substituted or unsubstituted, C₄ alkylene are independently replaced with —O—, —NR₅—, —S(═O)—, —S(═O)₂—, or —C(═O)—. In certain embodiments, L₃ is substituted or unsubstituted, C₅ alkylene, wherein one, two, or three backbone carbon atoms in the substituted or unsubstituted, C₅ alkylene are independently replaced with —O—, —NR₅—, —S(═O)—, —S(═O)₂—, or —C(═O)—. In certain embodiments, L₃ is substituted or unsubstituted, C₆ alkylene, wherein one, two, or three backbone carbon atoms in the substituted or unsubstituted, C₆ alkylene are independently replaced with —O—, —NR₅—, —S(═O)—, —S(═O)₂—, or —C(═O)—.

In certain embodiments, L₃ is substituted or unsubstituted, C₁₋₁₃ alkylene, substituted or unsubstituted, C₂₋₁₃ alkenylene, or substituted or unsubstituted, C₂₋₁₃ alkynylene, wherein one or more (e.g., two or three) backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, —S(═O)—, —S(═O)₂—, or —C(═O)—.

In certain embodiments, L₃ is substituted or unsubstituted, monocyclic heterocyclylene or substituted or unsubstituted, bicyclic heterocyclylene.

In certain embodiments, L₃ is -(substituted or unsubstituted, monocyclic heterocyclylene)-(substituted or unsubstituted, C₁ alkylene)-, -(substituted or unsubstituted, bicyclic heterocyclylene)-(substituted or unsubstituted, C₁ alkylene)-, -(substituted or unsubstituted, C₁ alkylene)-(substituted or unsubstituted, monocyclic heterocyclylene)-, —(substituted or unsubstituted, C₁ alkylene)-(substituted or unsubstituted, bicyclic heterocyclylene)-, -(substituted or unsubstituted, C₂ alkylene)-(substituted or unsubstituted, bicyclic carbocyclylene)-, -(substituted or unsubstituted, bicyclic carbocyclylene)-(substituted or unsubstituted, C₂ alkylene)-, optionally wherein one backbone carbon atom in the substituted or unsubstituted, C₁ alkylene or C₂ alkylene is replaced with —O—, —NR₅—, —S(═O)—, —S(═O)₂—, or —C(═O)—.

In certain embodiments, L₃ is -(substituted or unsubstituted, C₁ alkylene)-(substituted or unsubstituted, monocyclic heterocyclylene)-(substituted or unsubstituted, C₁ alkylene)- or -(substituted or unsubstituted, C₁ alkylene)-(substituted or unsubstituted, bicyclic heterocyclylene)-(substituted or unsubstituted, C₁ alkylene)-, optionally wherein the backbone carbon atom in each instance of the substituted or unsubstituted, C₁ alkylene is independently replaced with —O—, —NR₅—, —S(═O)—, —S(═O)₂—, or —C(═O)—.

In certain embodiments, L₃ is -(substituted or unsubstituted, C₁ alkylene)-(substituted or unsubstituted, monocyclic heterocyclylene)-or -(substituted or unsubstituted, monocyclic heterocyclylene)-(substituted or unsubstituted, C₁ alkylene)-.

In certain embodiments, L₃ is -(substituted or unsubstituted, monocyclic heterocyclylene)₂-, -(substituted or unsubstituted, monocyclic heterocyclylene)₂-(substituted or unsubstituted, C₁ alkylene)-, -(substituted or unsubstituted, C₁ alkylene)-(substituted or unsubstituted, monocyclic heterocyclylene)₂-, -(substituted or unsubstituted, C₁ alkylene)-(substituted or unsubstituted, monocyclic heterocyclylene)₂-(substituted or unsubstituted, C₁ alkylene)-, -(substituted or unsubstituted, monocyclic heterocyclylene)-(substituted or unsubstituted, monocyclic carbocyclylene)-, or -(substituted or unsubstituted, monocyclic carbocyclylene)-(substituted or unsubstituted, monocyclic heterocyclylene)-, optionally wherein the backbone carbon atom in each instance of the substituted or unsubstituted, C₁ alkylene is independently replaced with —O—, —NR₅—, —S(═O)—, —S(═O)₂—, or —C(═O)—.

In certain embodiments, L₃ is

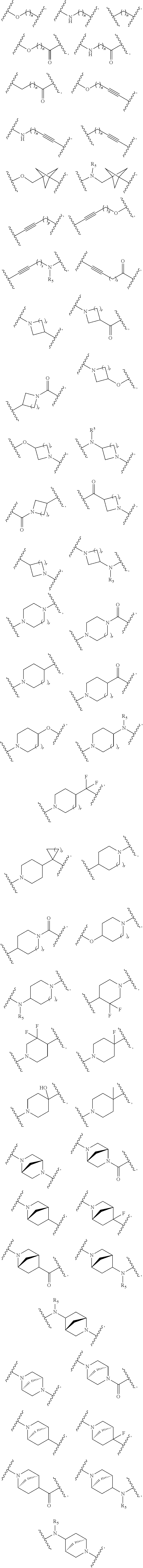

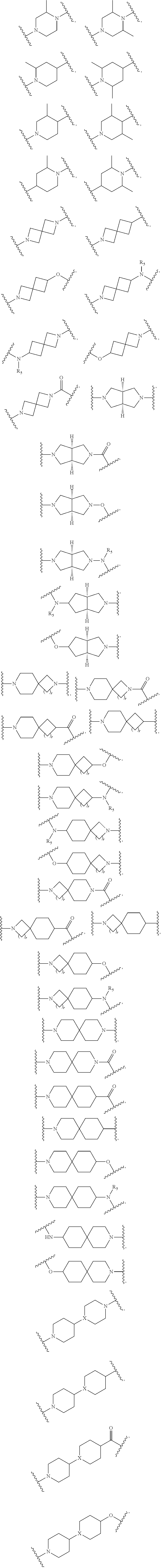

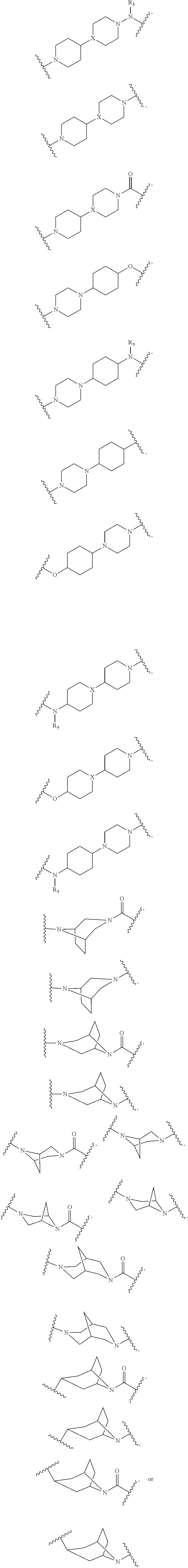

In certain embodiments, L₃ is attached to the 2 or 3 position. In certain embodiments, L₃ is attached to the 1 or 4 position.

In certain embodiments, the compound is of the formula:

| Com- | |
|---|---|
| pound | |
| Num- | |
| ber | Formula |
| 1 | 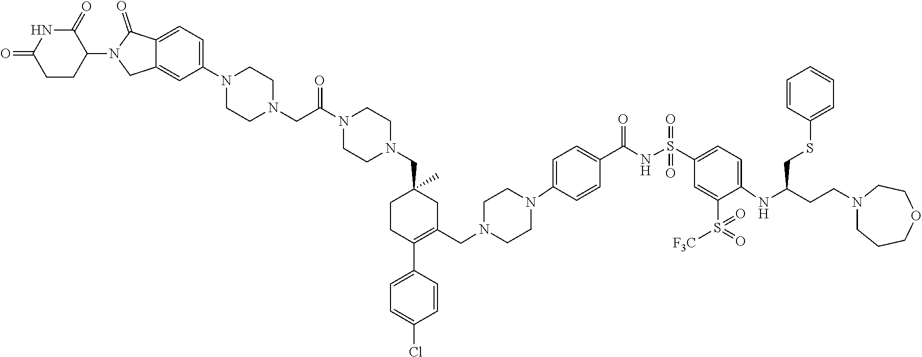 [SMILES: C[C@@]1(CN2CCN(C(=O)CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 2 | 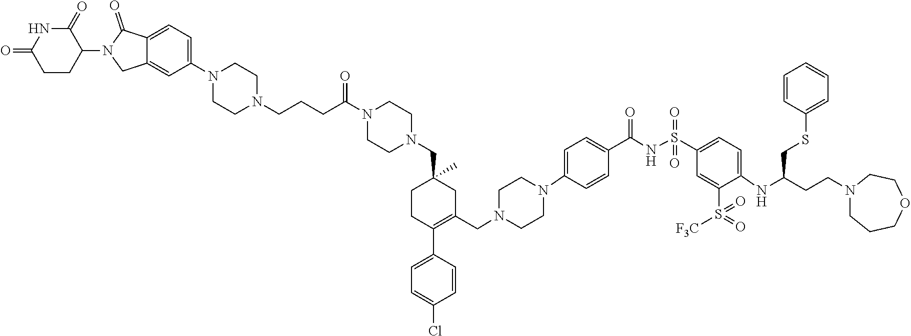 [SMILES: C[C@@]1(CN2CCN(C(=O)CCCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 3 | 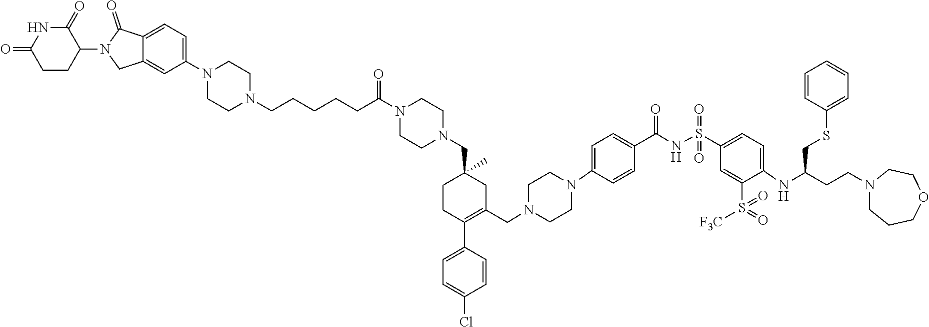 [SMILES: C[C@@]1(CN2CCN(C(=O)CCCCCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 4 | 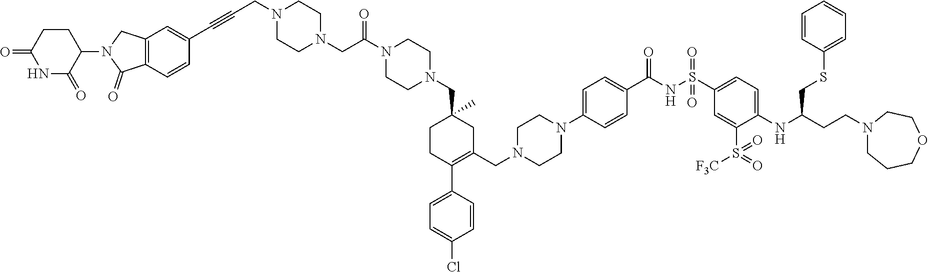 [SMILES: C[C@@]1(CN2CCN(C(=O)CN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 5 | 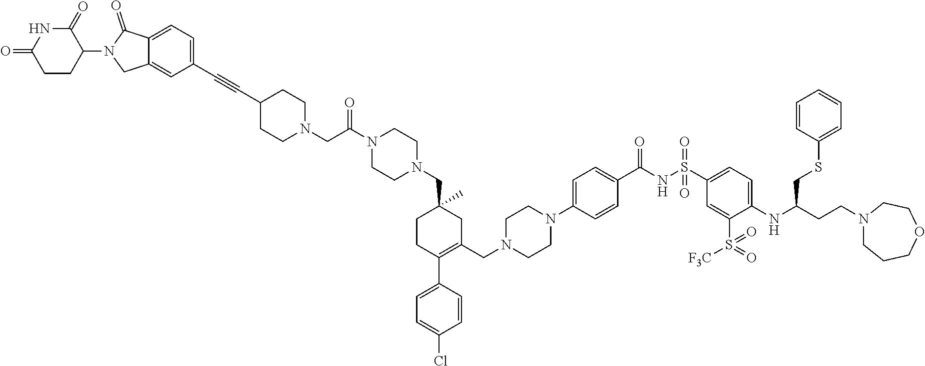 [SMILES: C[C@@]1(CN2CCN(C(=O)CN3CCC(C#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 6 | 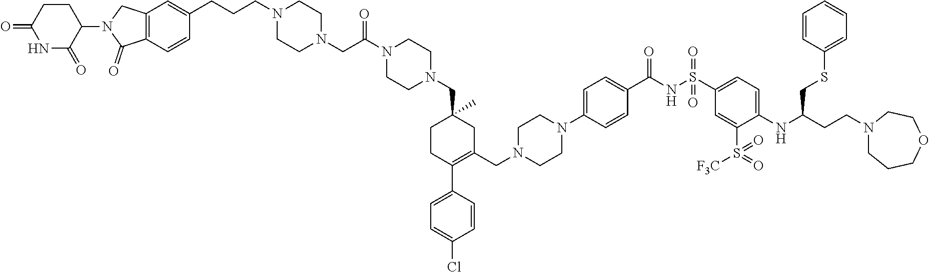 [SMILES: C[C@@]1(CN2CCN(C(=O)CN3CCN(CCCc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 7 | 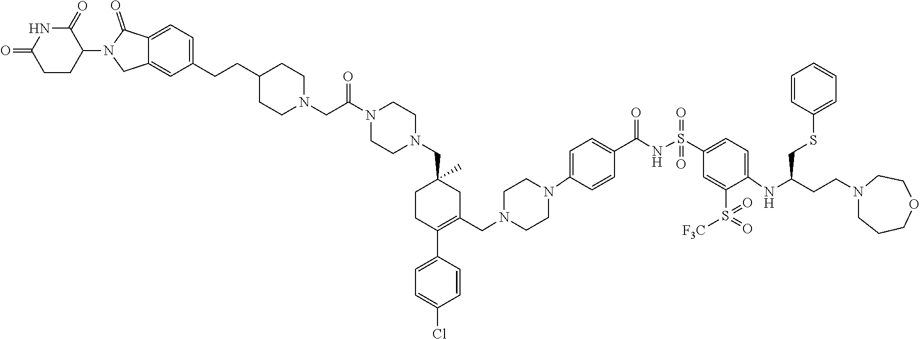 [SMILES: C[C@@]1(CN2CCN(C(=O)CN3CCC(CCc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 8 | 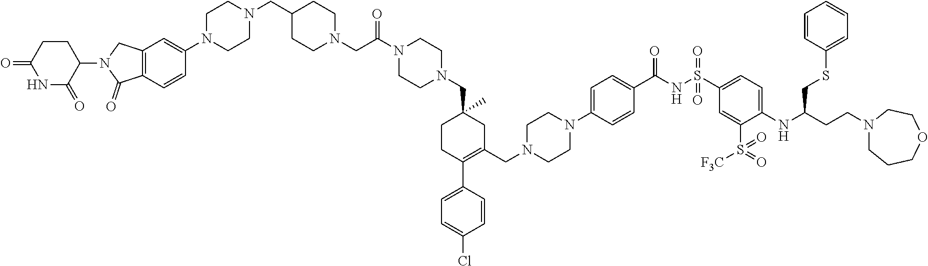 [SMILES: C[C@@]1(CN2CCN(C(=O)CN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 9 | 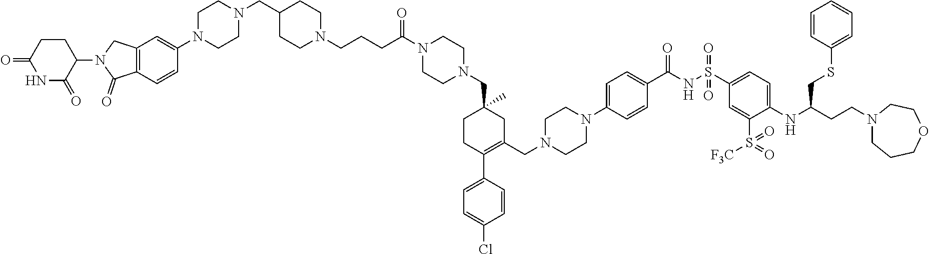 [SMILES: C[C@@]1(CN2CCN(C(=O)CCCN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 10 | 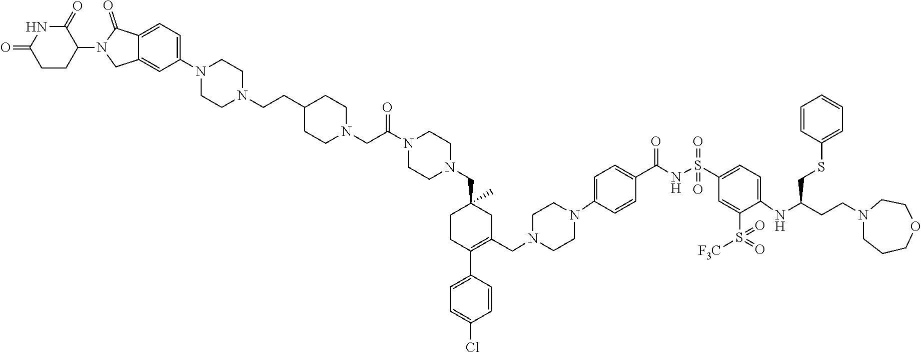 [SMILES: C[C@@]1(CN2CCN(C(=O)CN3CCC(CCN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 11 | 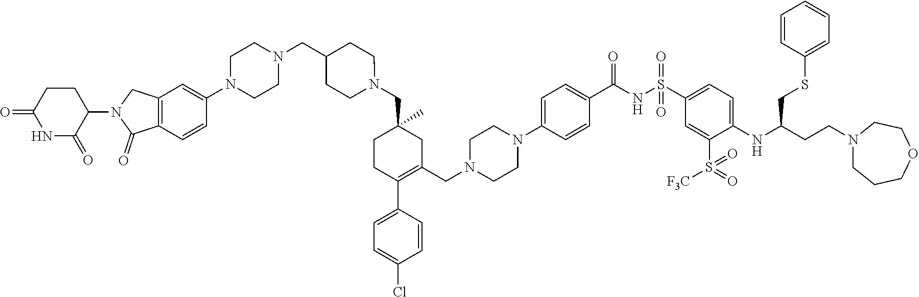 [SMILES: C[C@@]1(CN2CCC(CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 12 | 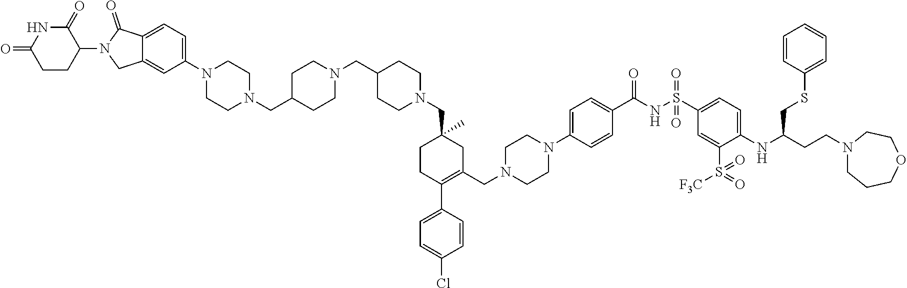 [SMILES: C[C@@]1(CN2CCC(CN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 13 | 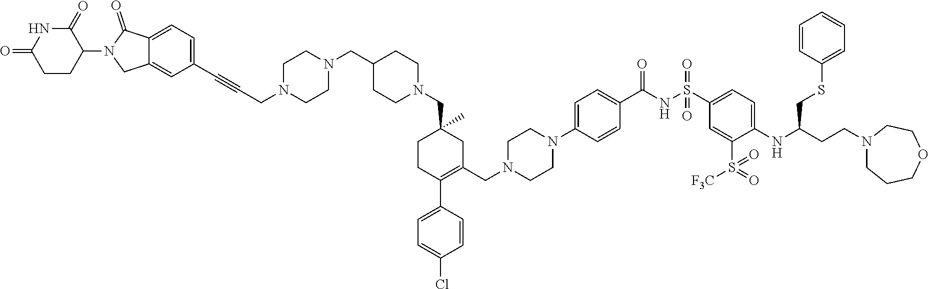 [SMILES: C[C@@]1(CN2CCC(CN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 14 | 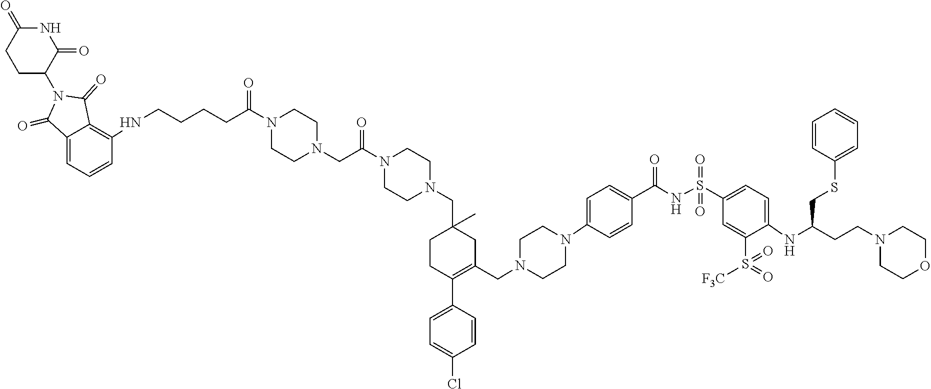 [SMILES: CC1(CN2CCN(C(=O)CN3CCN(C(=O)CCCCNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 15 | 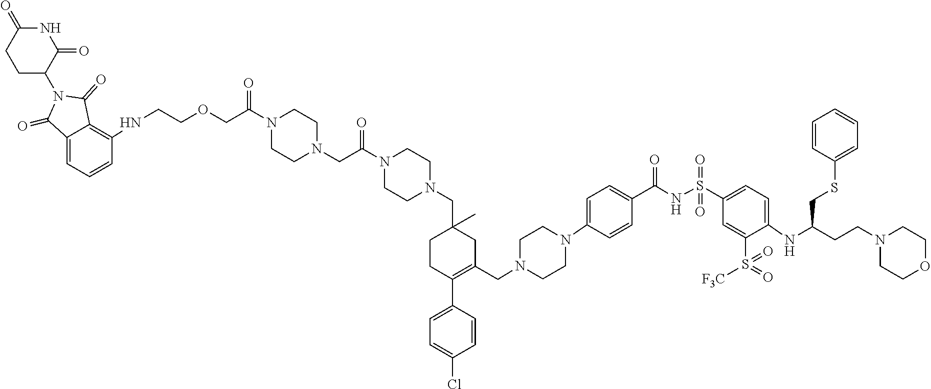 [SMILES: CC1(CN2CCN(C(=O)CN3CCN(C(=O)COCCNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 16 | 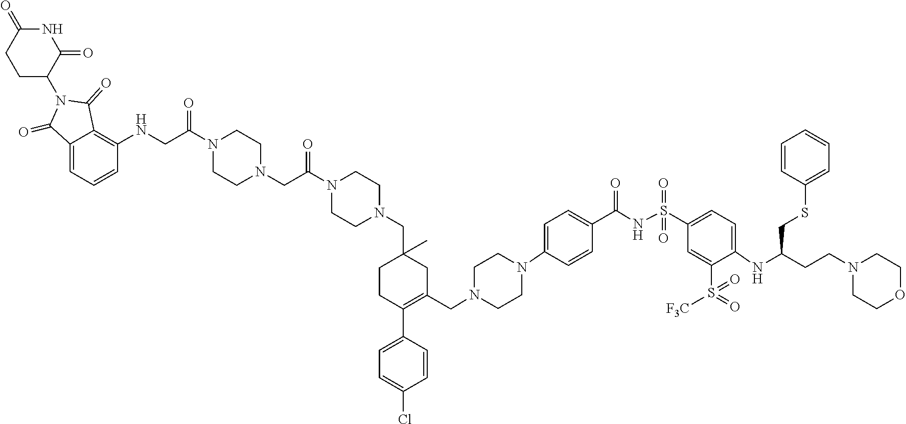 [SMILES: CC1(CN2CCN(C(=O)CN3CCN(C(=O)CNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 17 | 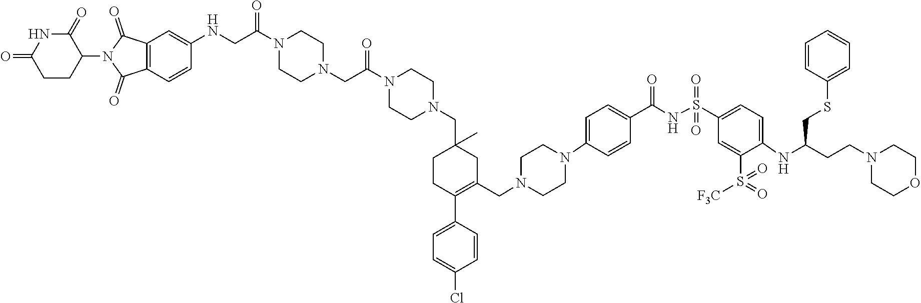 [SMILES: CC1(CN2CCN(C(=O)CN3CCN(C(=O)CNc4ccc5c(c4)C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 18 | 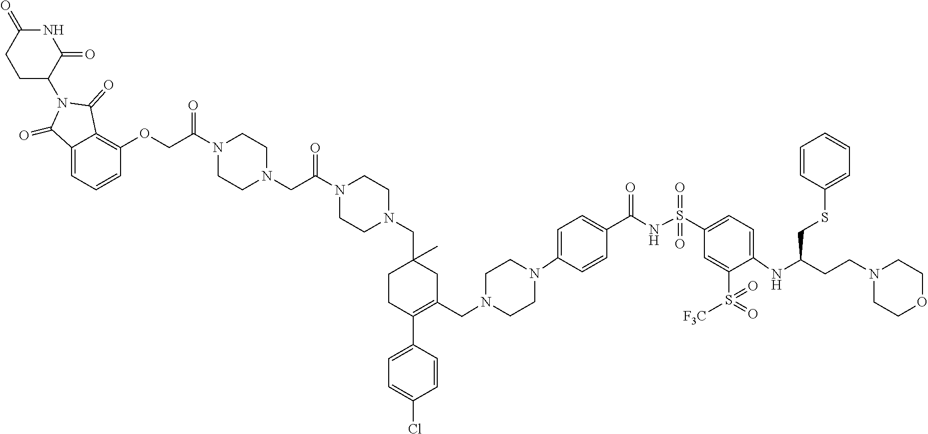 [SMILES: CC1(CN2CCN(C(=O)CN3CCN(C(=O)COc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 19 | 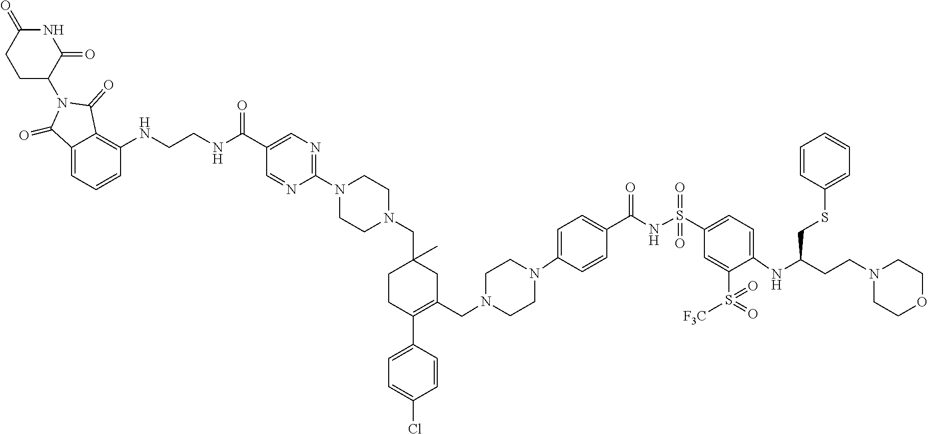 [SMILES: CC1(CN2CCN(c3ncc(C(=O)NCCNc4cccc5c4C(=O)N(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 20 | 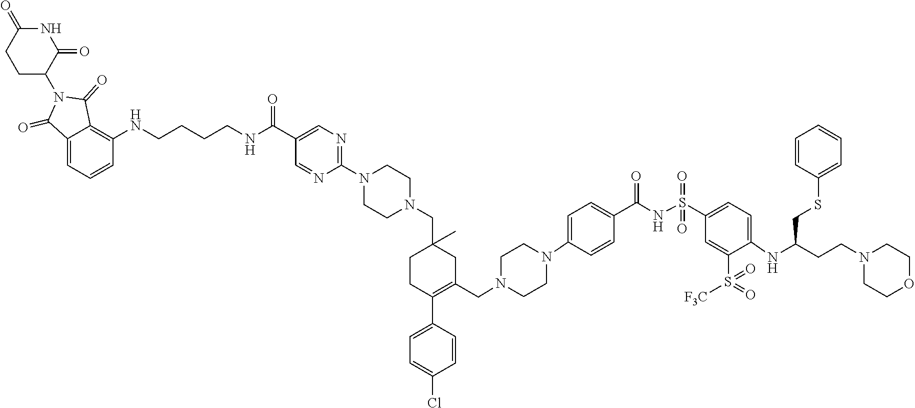 [SMILES: CC1(CN2CCN(c3ncc(C(=O)NCCCCNc4cccc5c4C(=O)N(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 21 | 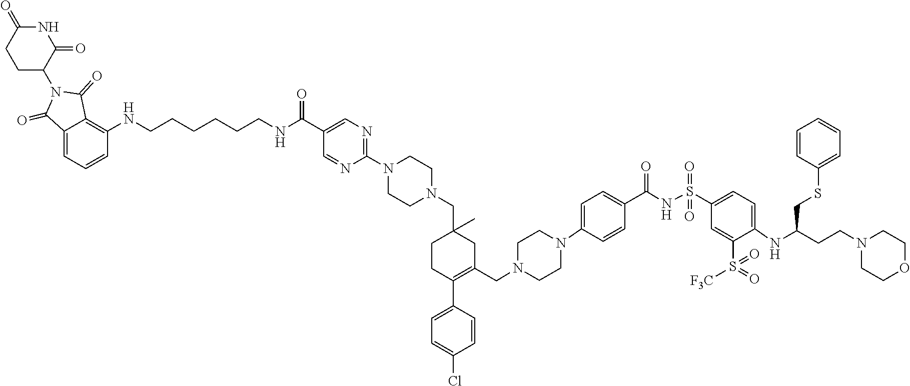 [SMILES: CC1(CN2CCN(c3ncc(C(=O)NCCCCCCNc4cccc5c4C(=O)N(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 22 | 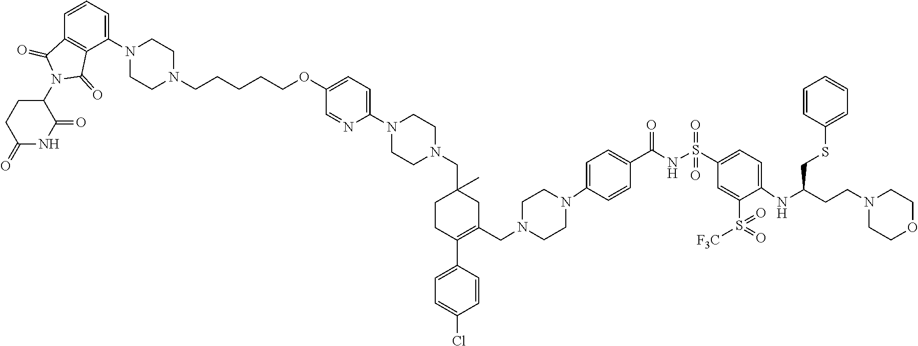 [SMILES: CC1(CN2CCN(c3ccc(OCCCCCN4CCN(c5cccc6c5C(=O)N(C5CCC(=O)NC5=O)C6=O)CC4)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 23 | 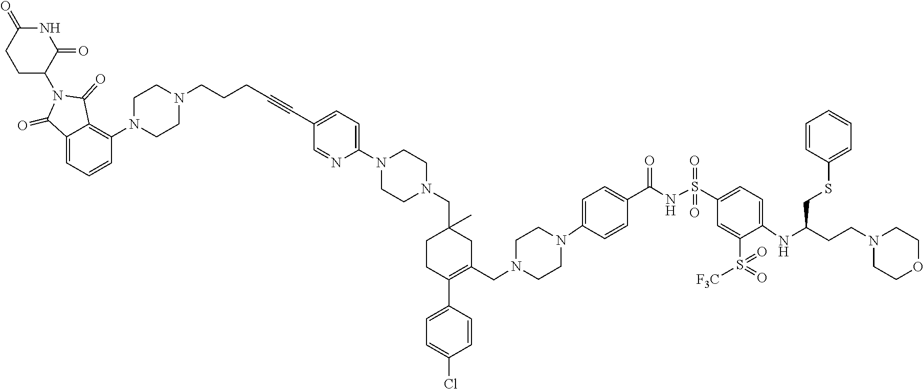 [SMILES: CC1(CN2CCN(c3ccc(C#CCCCN4CCN(c5cccc6c5C(=O)N(C5CCC(=O)NC5=O)C6=O)CC4)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 24 | 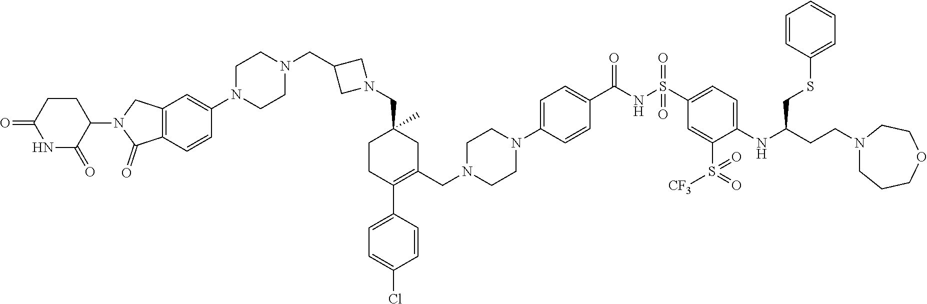 [SMILES: C[C@@]1(CN2CC(CN3CCN(c4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 25 | 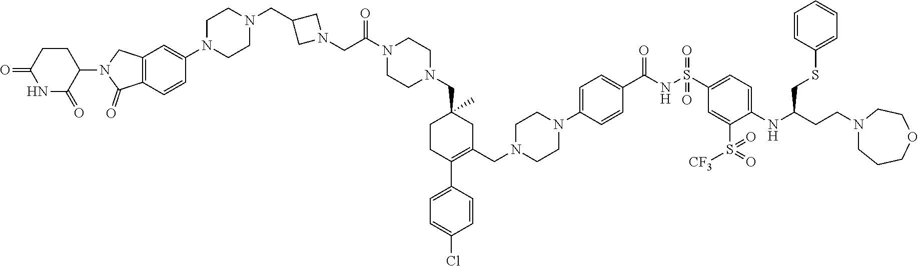 [SMILES: C[C@@]1(CN2CCN(C(=O)CN3CC(CN4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 26 | 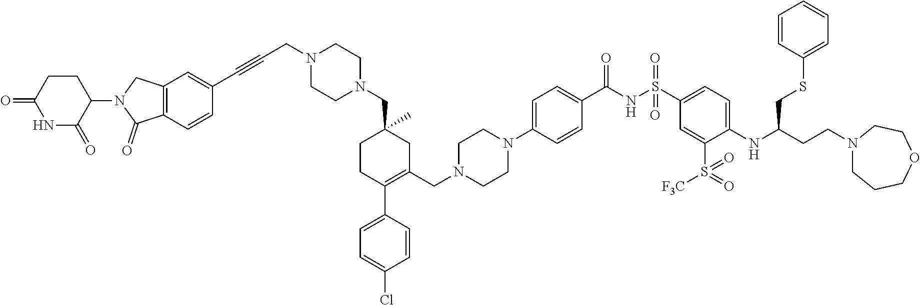 [SMILES: C[C@@]1(CN2CCN(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 27 | 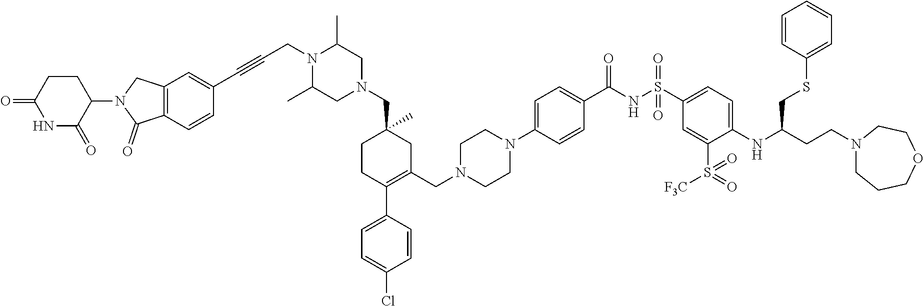 [SMILES: CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC(C)N1CC#Cc1ccc2c(c1)CN(C1CCC(=O)NC1=O)C2=O] |
| 28 | 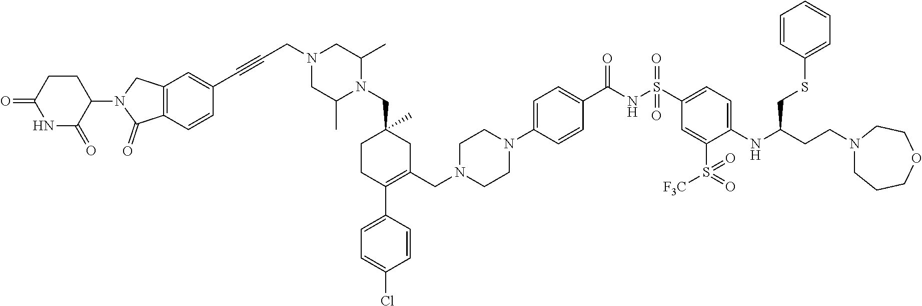 [SMILES: CC1CN(CC#Cc2ccc3c(c2)CN(C2CCC(=O)NC2=O)C3=O)CC(C)N1C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 29 | 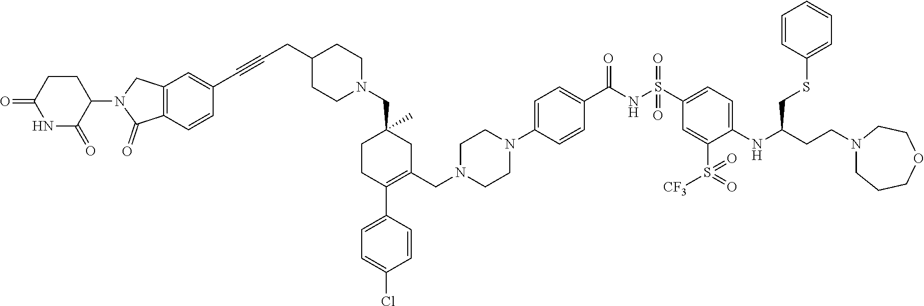 [SMILES: C[C@@]1(CN2CCC(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 30 | 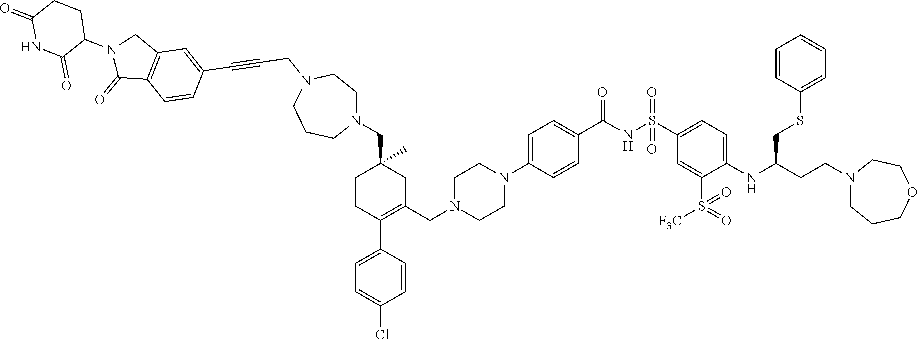 [SMILES: C[C@@]1(CN2CCCN(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 31 | 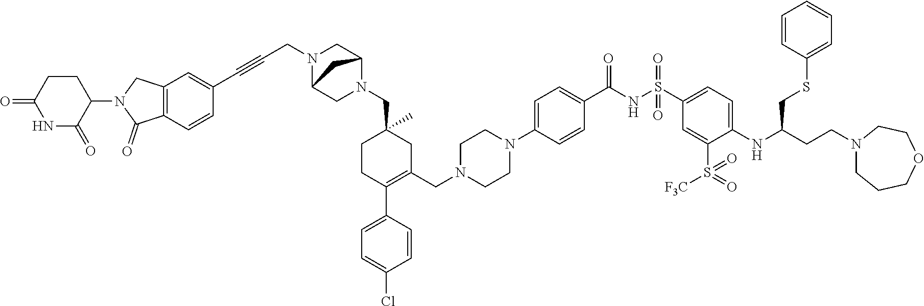 [SMILES: C[C@@]1(CN2C[C@H]3C[C@@H]2CN3CC#Cc2ccc3c(c2)CN(C2CCC(=O)NC2=O)C3=O)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 32 | 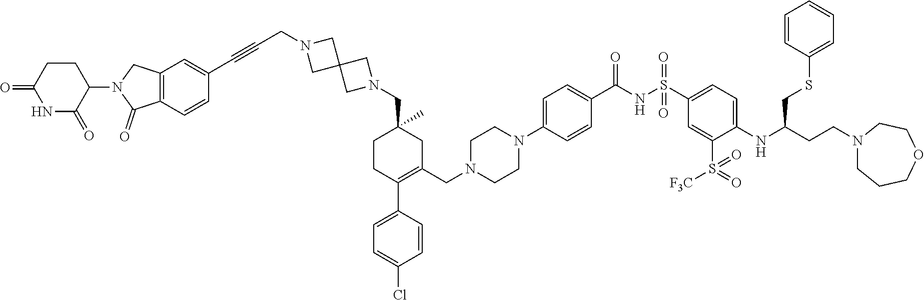 [SMILES: C[C@@]1(CN2CC3(CN(CC#Cc4ccc5c(c4)CN(C4CCC(=O)NC4=O)C5=O)C3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 33 | 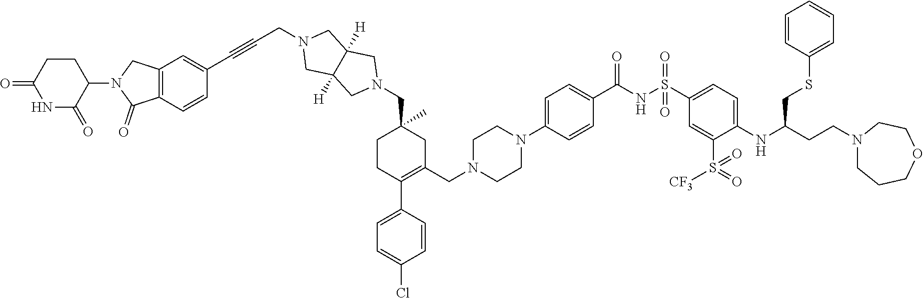 [SMILES: C[C@@]1(CN2C[C@H]3CN(CC#Cc4ccc5c(c4)CN(C4CCC(=O)NC4=O)C5=O)C[C@H]3C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 34 | 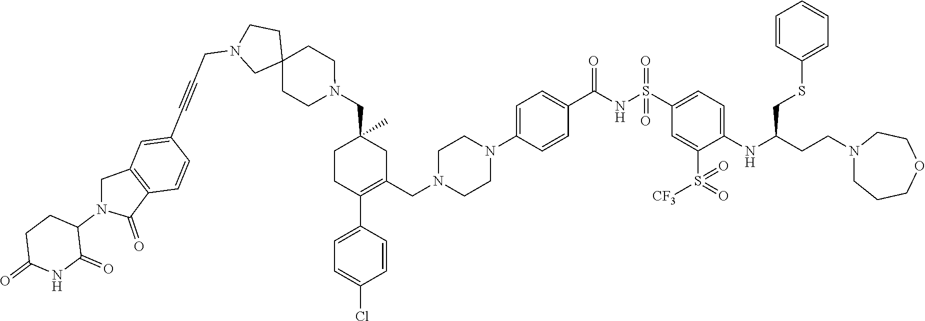 [SMILES: C[C@@]1(CN2CCC3(CCN(CC#Cc4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 35 | 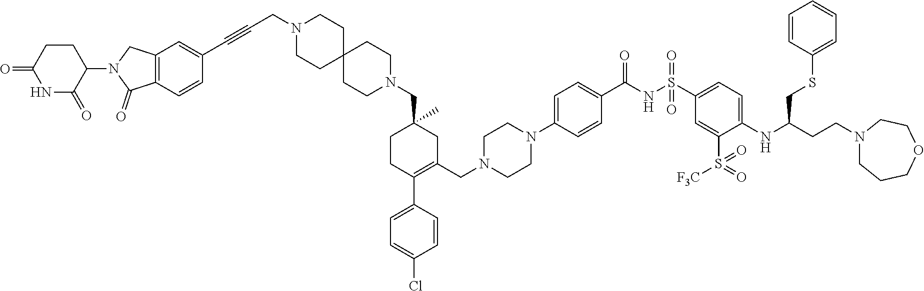 [SMILES: C[C@@]1(CN2CCC3(CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 36 | 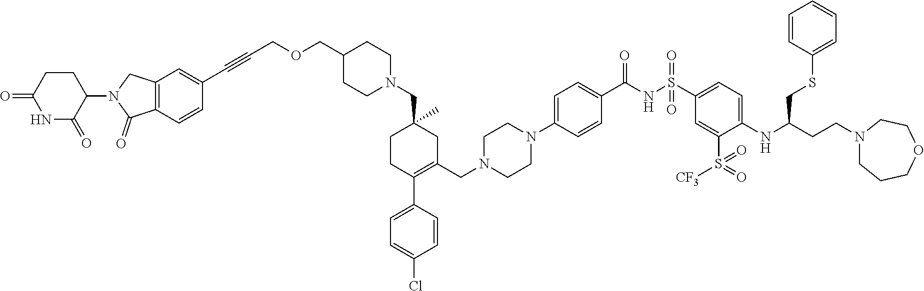 [SMILES: C[C@@]1(CN2CCC(COCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 37 | 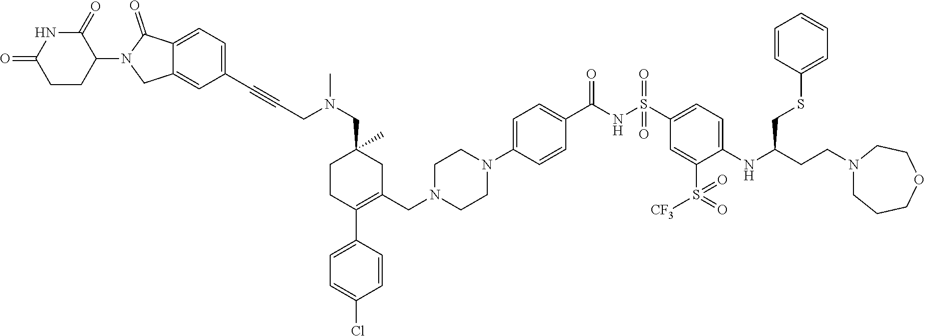 [SMILES: CN(CC#Cc1ccc2c(c1)CN(C1CCC(=O)NC1=O)C2=O)C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 38 | 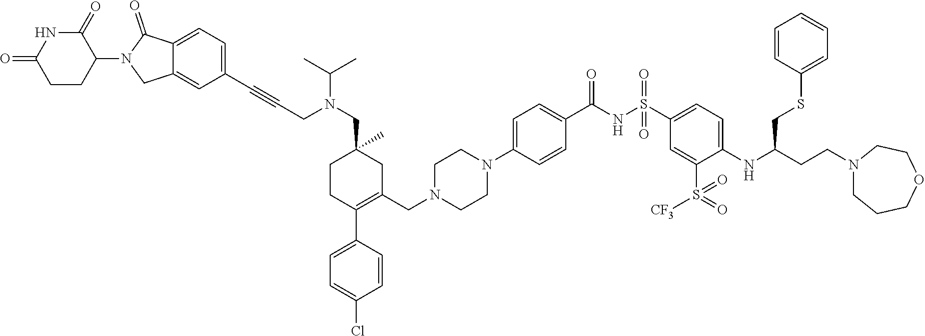 [SMILES: CC(C)N(CC#Cc1ccc2c(c1)CN(C1CCC(=O)NC1=O)C2=O)C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 39 | 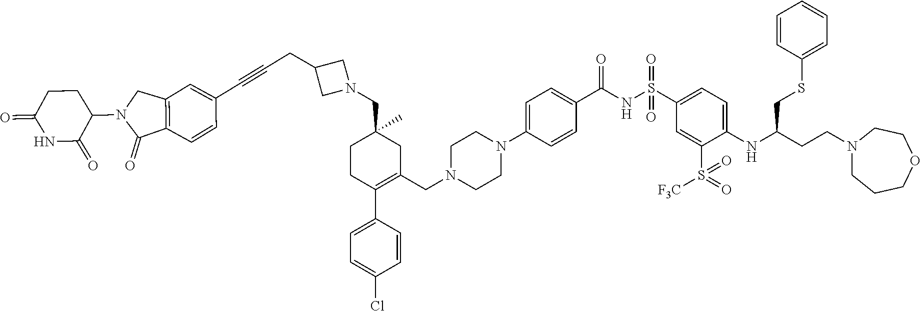 [SMILES: C[C@@]1(CN2CC(CC#Cc3ccc4c(c3)CN(C3CCC(=O)NC3=O)C4=O)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 40 | 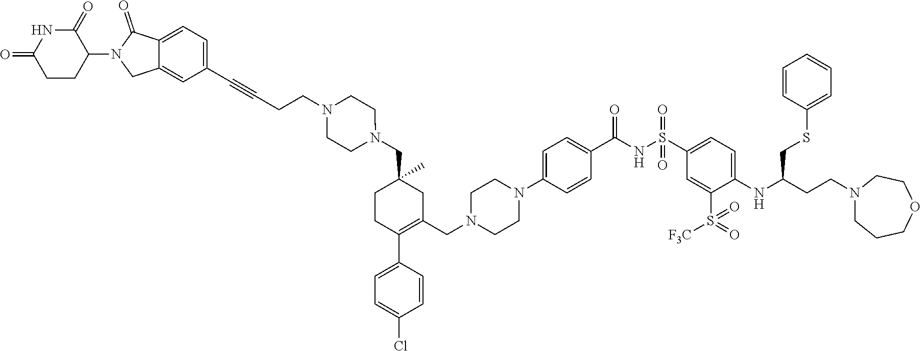 [SMILES: C[C@@]1(CN2CCN(CCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 41 | 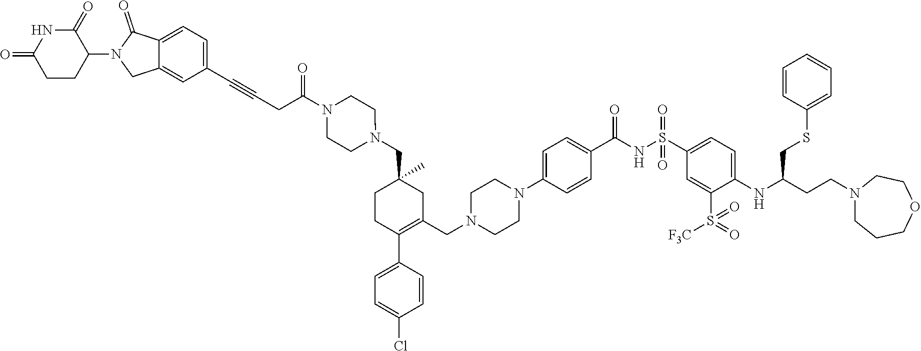 [SMILES: C[C@@]1(CN2CCN(C(=O)CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 42 | 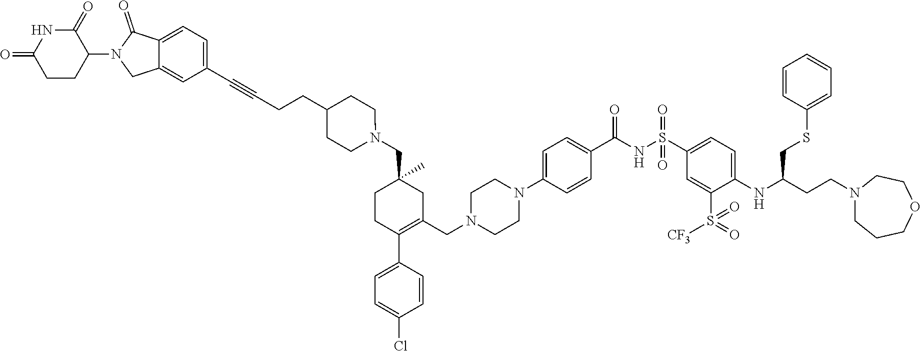 [SMILES: C[C@@]1(CN2CCC(CCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 43 | 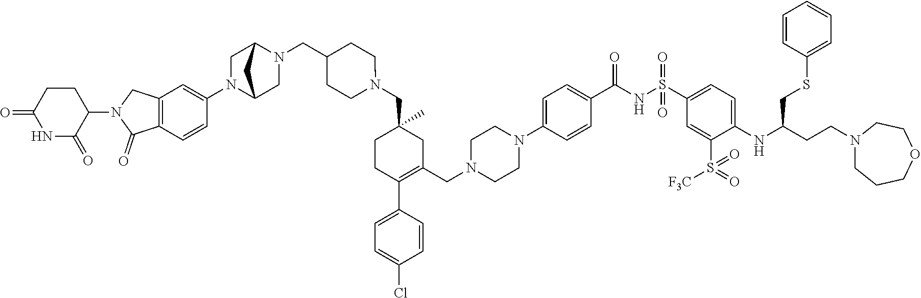 [SMILES: C[C@@]1(CN2CCC(CN3C[C@H]4C[C@@H]3CN4c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 44 | 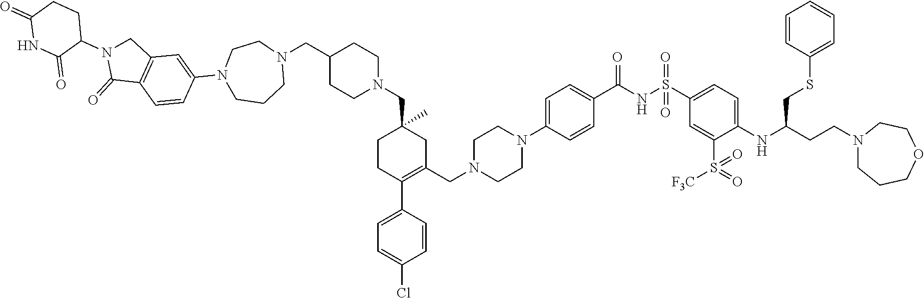 [SMILES: C[C@@]1(CN2CCC(CN3CCCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 45 | 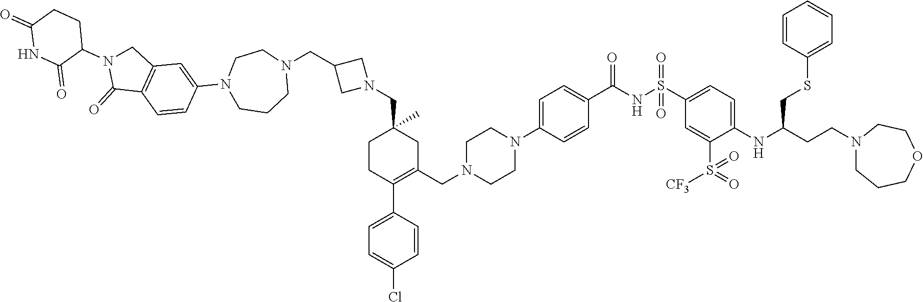 [SMILES: C[C@@]1(CN2CC(CN3CCCN(c4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 46 | 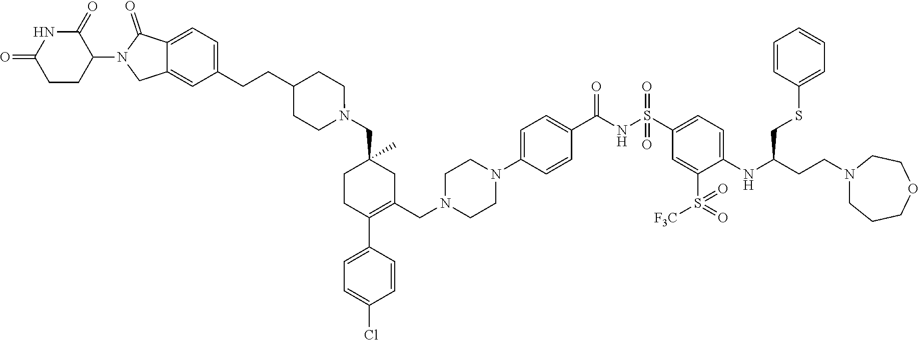 [SMILES: C[C@@]1(CN2CCC(CCc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 47 | 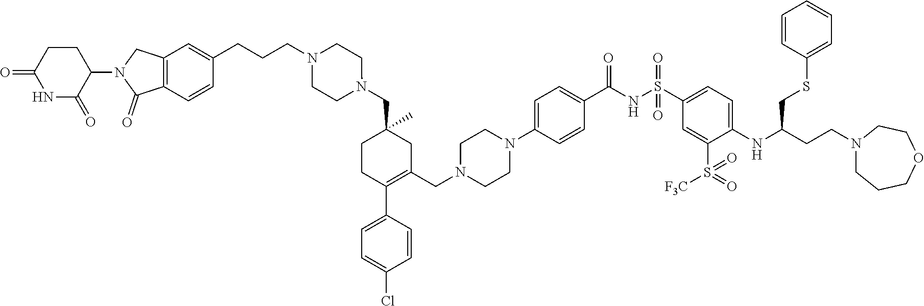 [SMILES: C[C@@]1(CN2CCN(CCCc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 48 | 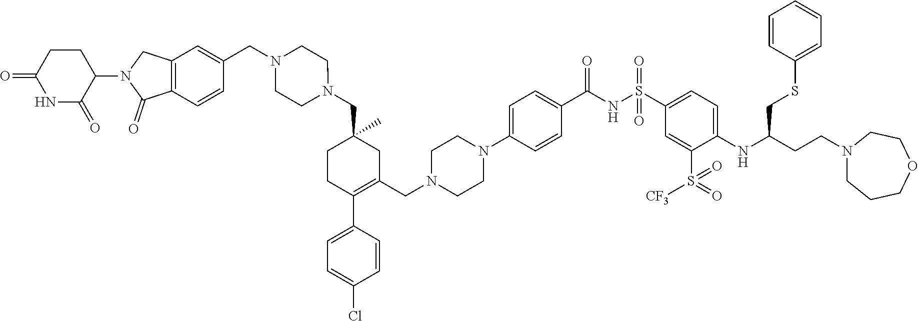 [SMILES: C[C@@]1(CN2CCN(Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 49 | 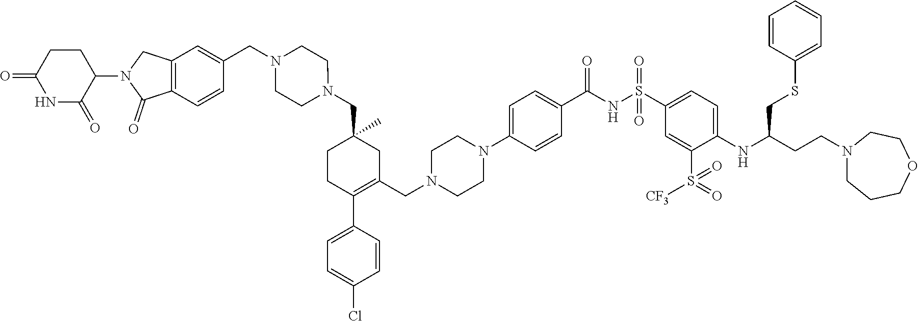 [SMILES: C[C@@]1(CN2CCN(Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 50 | 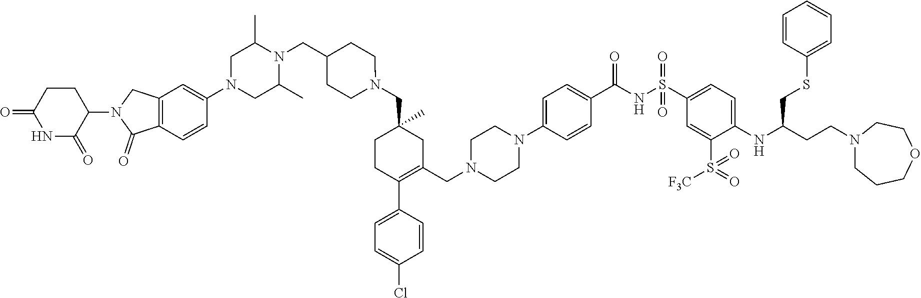 [SMILES: CC1CN(c2ccc3c(c2)CN(C2CCC(=O)NC2=O)C3=O)CC(C)N1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 51 | 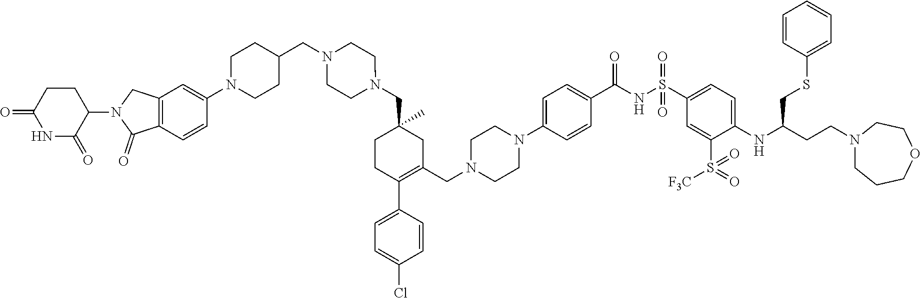 [SMILES: C[C@@]1(CN2CCN(CC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 52 | 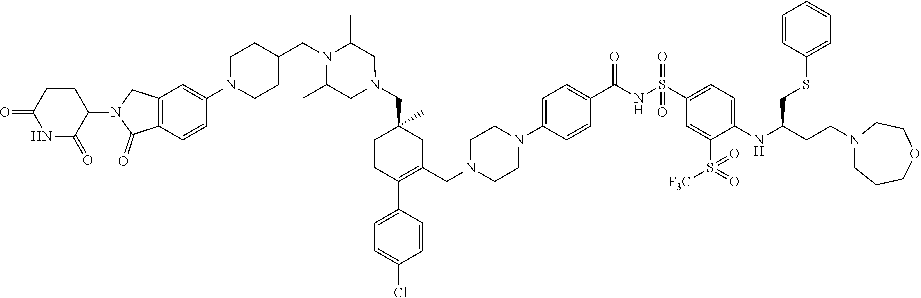 [SMILES: CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC(C)N1CC1CCN(c2ccc3c(c2)CN(C2CCC(=O)NC2=O)C3=O)CC1] |
| 53 | 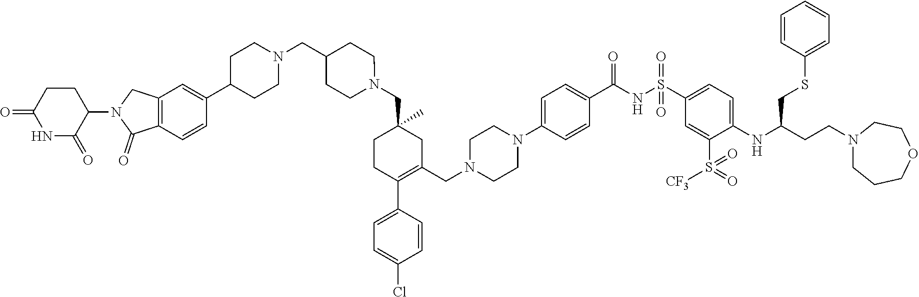 [SMILES: C[C@@]1(CN2CCC(CN3CCC(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 54 | 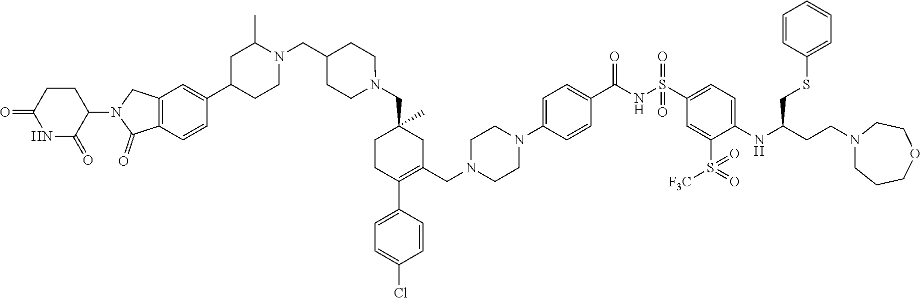 [SMILES: CC1CC(c2ccc3c(c2)CN(C2CCC(=O)NC2=O)C3=O)CCN1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 55 | 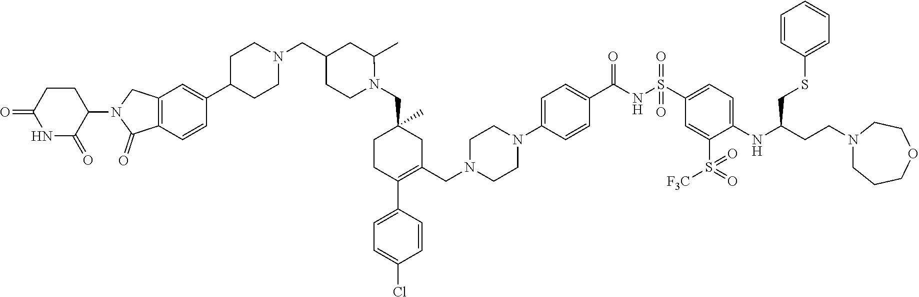 [SMILES: CC1CC(CN2CCC(c3ccc4c(c3)CN(C3CCC(=O)NC3=O)C4=O)CC2)CCN1C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 56 | 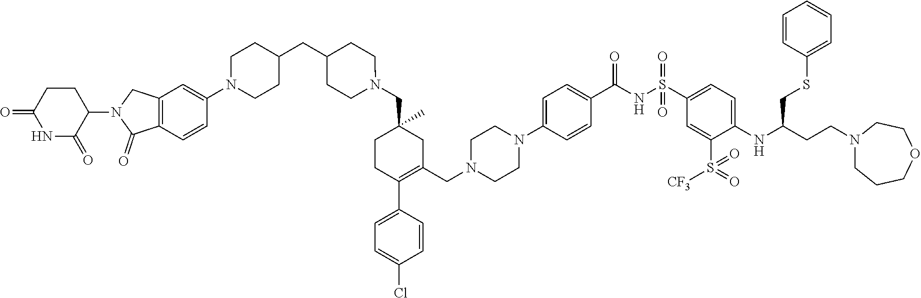 [SMILES: C[C@@]1(CN2CCC(CC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 57 | 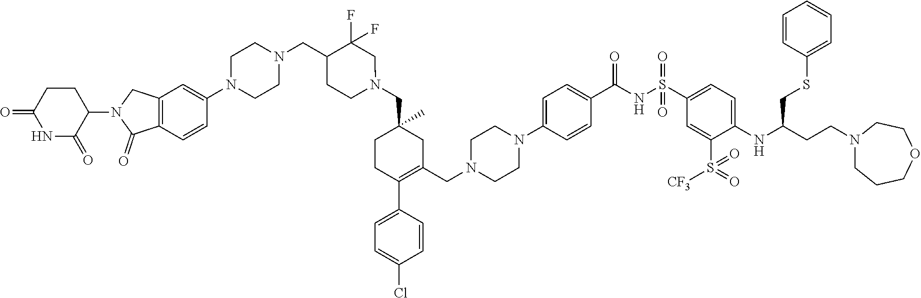 [SMILES: C[C@@]1(CN2CCC(CN3CCN(c4ccc5c(c4)CN(C4CCC(=O)NC4=O)C5=O)CC3)C(F)(F)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 58 | 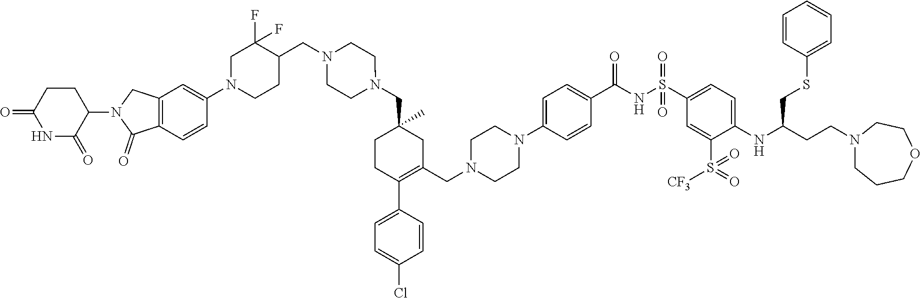 [SMILES: C[C@@]1(CN2CCN(CC3CCN(c4ccc5c(c4)CN(C4CCC(=O)NC4=O)C5=O)CC3(F)F)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 59 | 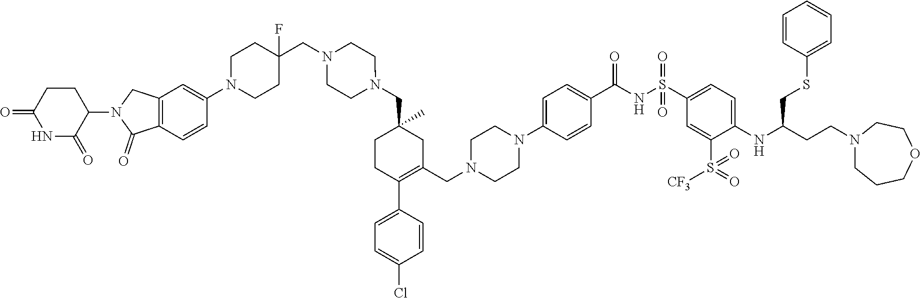 [SMILES: C[C@@]1(CN2CCN(CC3(F)CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 60 | 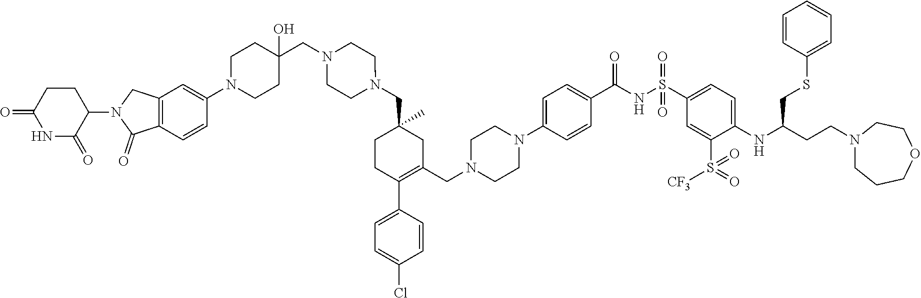 [SMILES: C[C@@]1(CN2CCN(CC3(O)CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 61 | 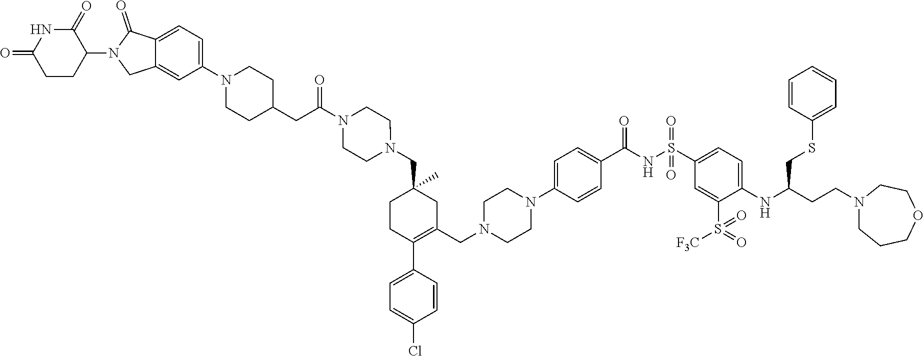 [SMILES: C[C@@]1(CN2CCN(C(=O)CC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 62 | 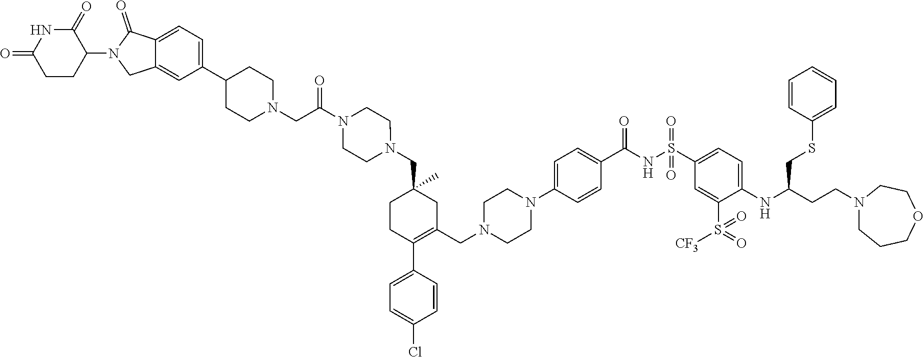 [SMILES: C[C@@]1(CN2CCN(C(=O)CN3CCC(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 63 | 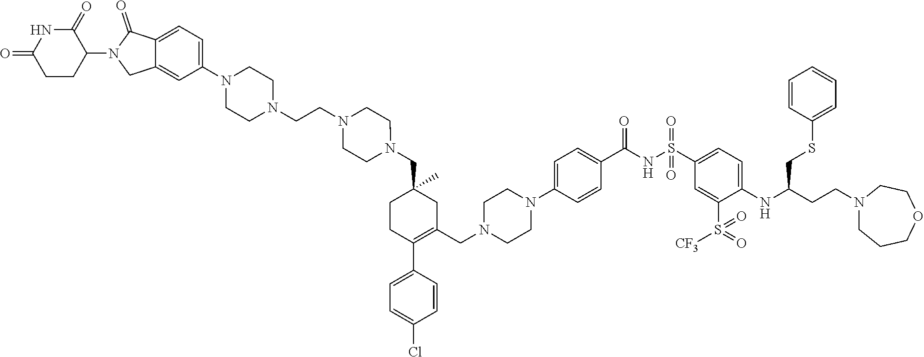 [SMILES: C[C@@]1(CN2CCN(CCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 64 | 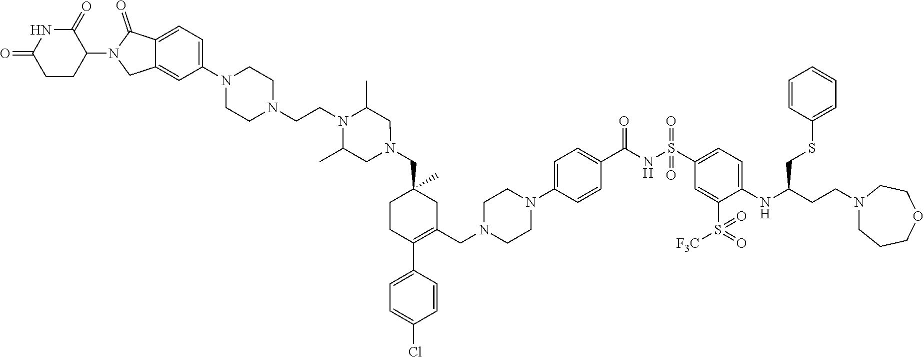 [SMILES: CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC(C)N1CCN1CCN(c2ccc3c(c2)CN(C2CCC(=O)NC2=O)C3=O)CC1] |
| 65 | 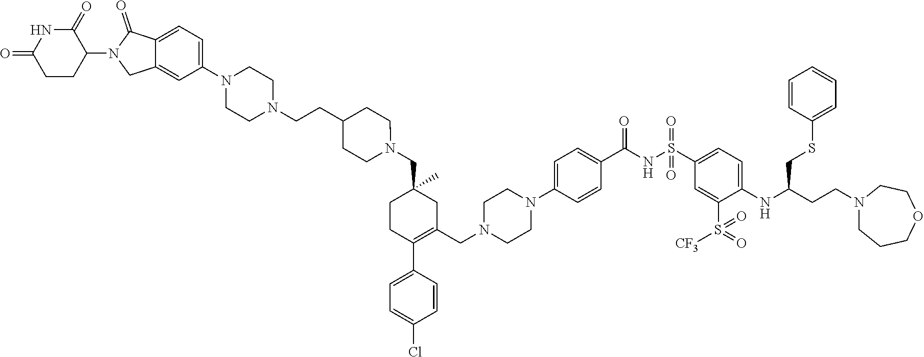 [SMILES: C[C@@]1(CN2CCC(CCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 66 | 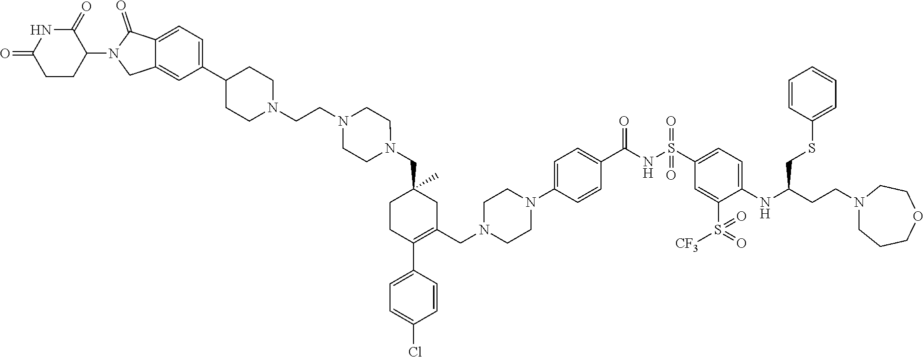 [SMILES: C[C@@]1(CN2CCN(CCN3CCC(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 67 | 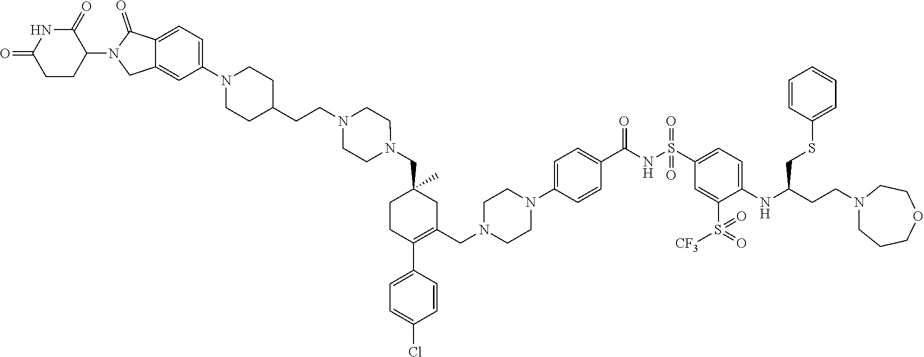 [SMILES: C[C@@]1(CN2CCN(CCC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 68 | 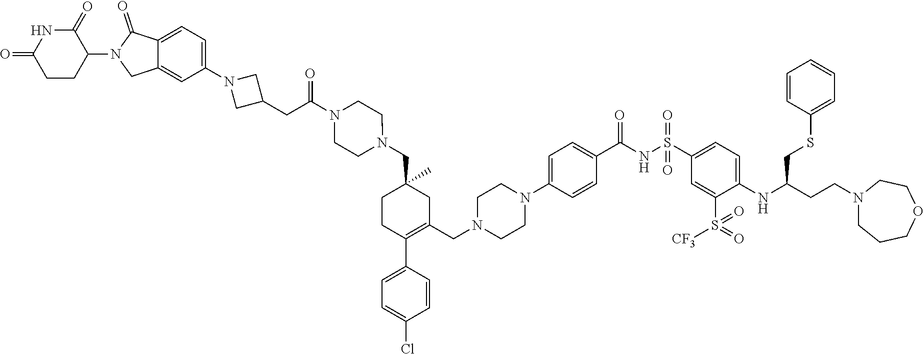 [SMILES: C[C@@]1(CN2CCN(C(=O)CC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 69 | 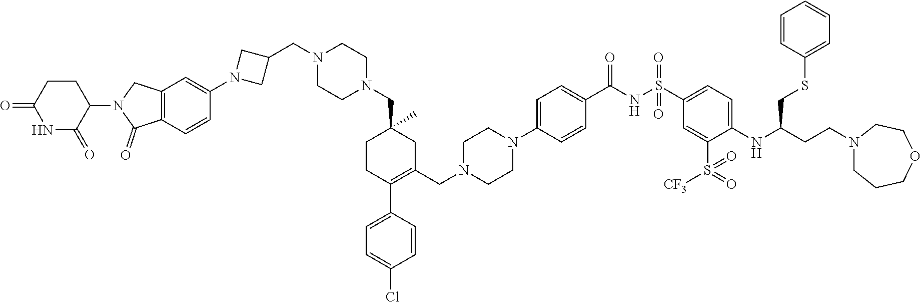 [SMILES: C[C@@]1(CN2CCN(CC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 70 | 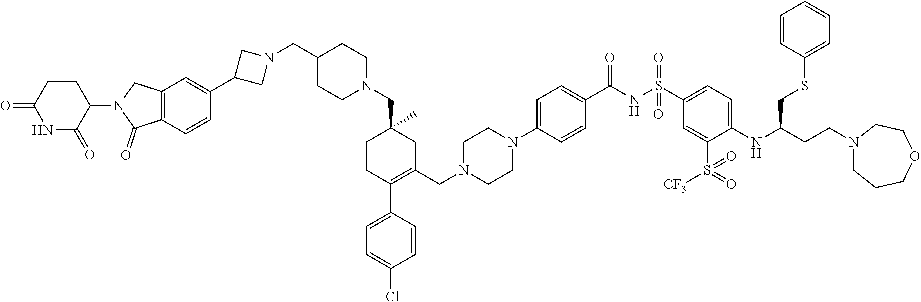 [SMILES: C[C@@]1(CN2CCC(CN3CC(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 71 | 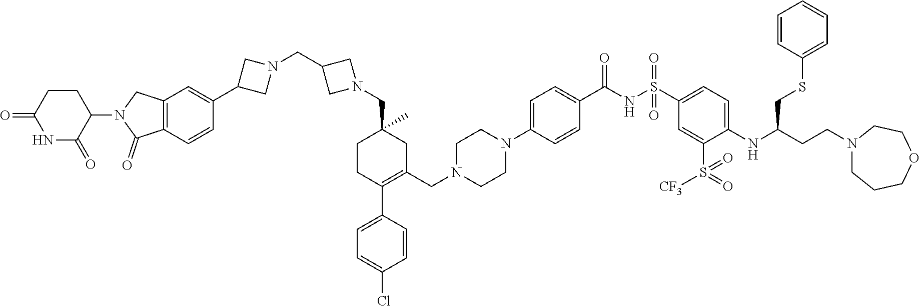 [SMILES: C[C@@]1(CN2CC(CN3CC(c4ccc5c(c4)CN(C4CCC(=O)NC4=O)C5=O)C3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 72 | 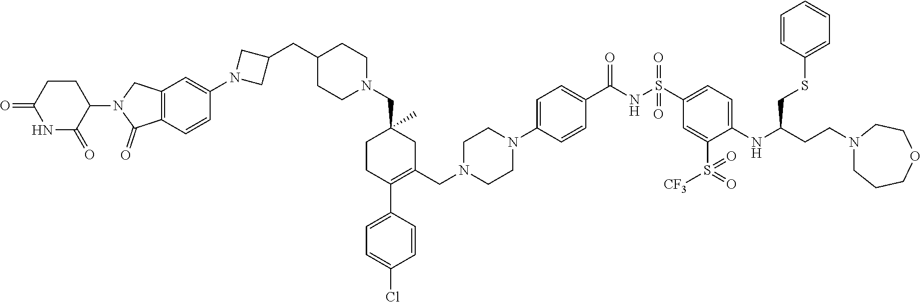 [SMILES: C[C@@]1(CN2CCC(CC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 73 | 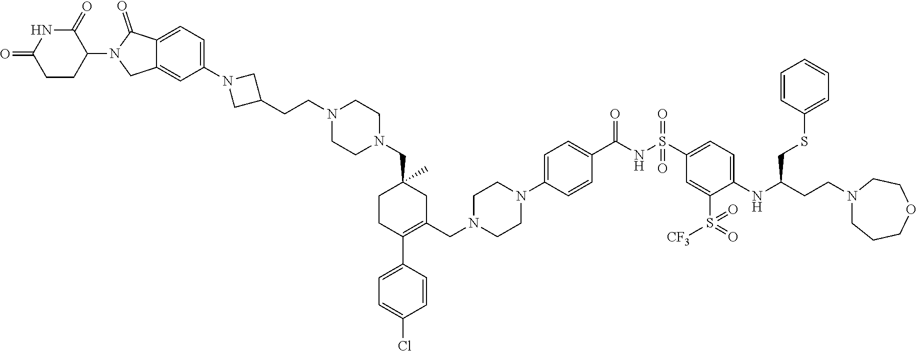 [SMILES: C[C@@]1(CN2CCN(CCC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 74 | 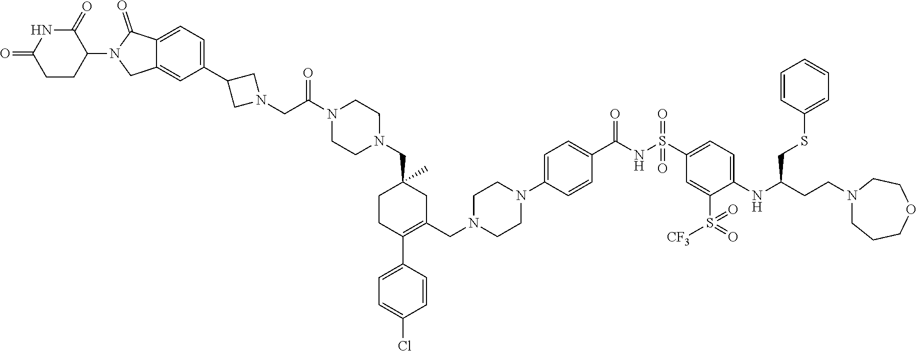 [SMILES: C[C@@]1(CN2CCN(C(=O)CN3CC(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 75 | 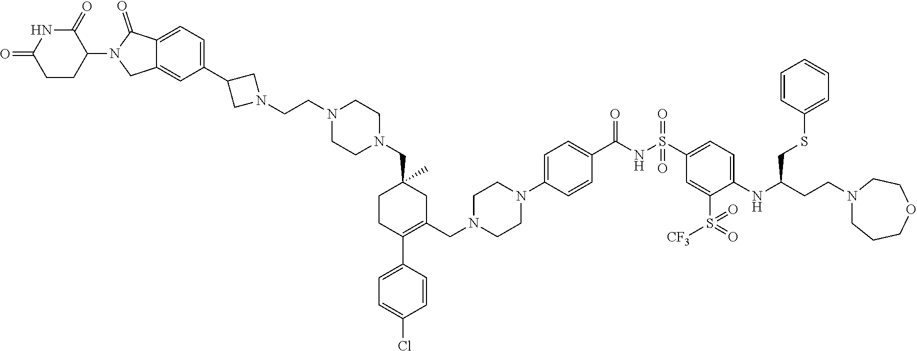 [SMILES: C[C@@]1(CN2CCN(CCN3CC(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 76 | 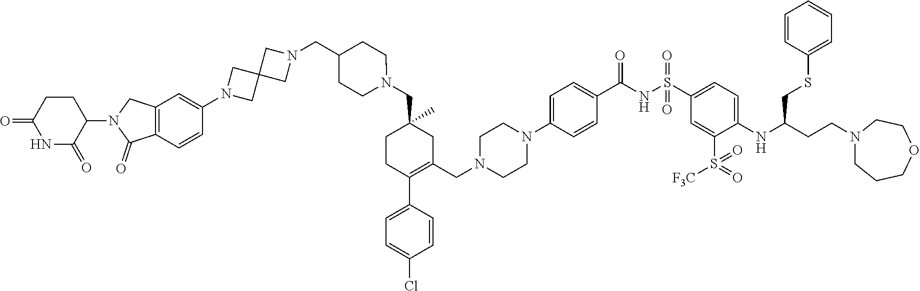 [SMILES: C[C@@]1(CN2CCC(CN3CC4(C3)CN(c3ccc5c(c3)CN(C3CCC(=O)NC3=O)C5=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 77 | 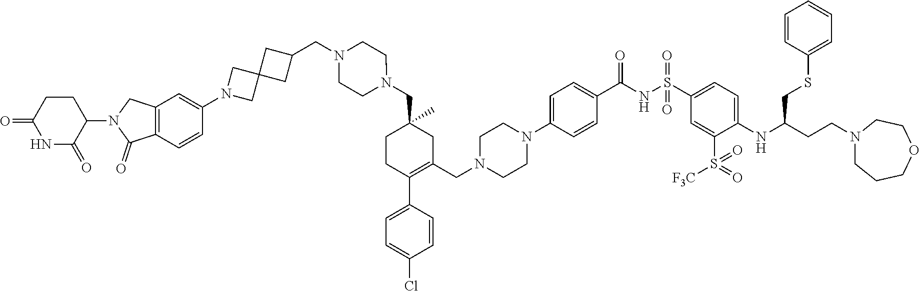 [SMILES: C[C@@]1(CN2CCN(CC3CC4(C3)CN(c3ccc5c(c3)CN(C3CCC(=O)NC3=O)C5=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 78 | 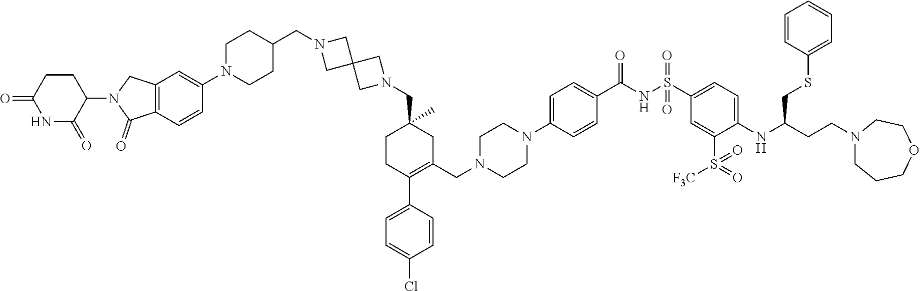 [SMILES: C[C@@]1(CN2CC3(CN(CC4CCN(c5ccc6c(c5)CN(C5CCC(=O)NC5=O)C6=O)CC4)C3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 79 | 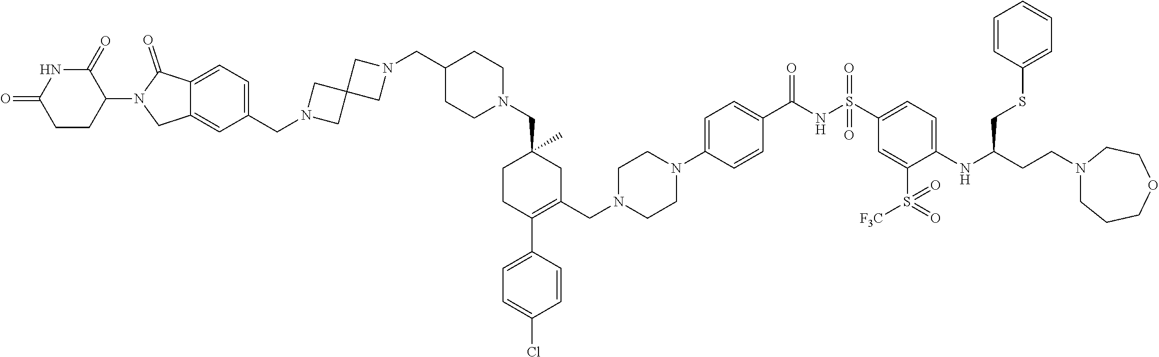 [SMILES: C[C@@]1(CN2CCC(CN3CC4(CN(Cc5ccc6c(c5)CN(C5CCC(=O)NC5=O)C6=O)C4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 80 | 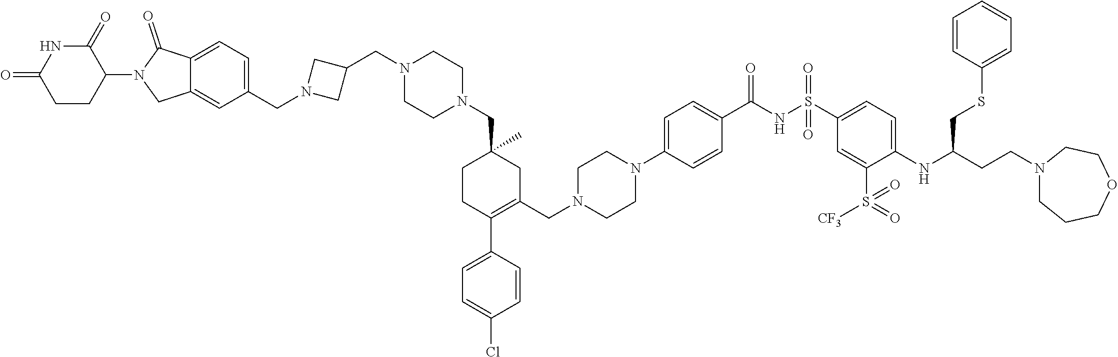 [SMILES: C[C@@]1(CN2CCN(CC3CN(Cc4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 81 | 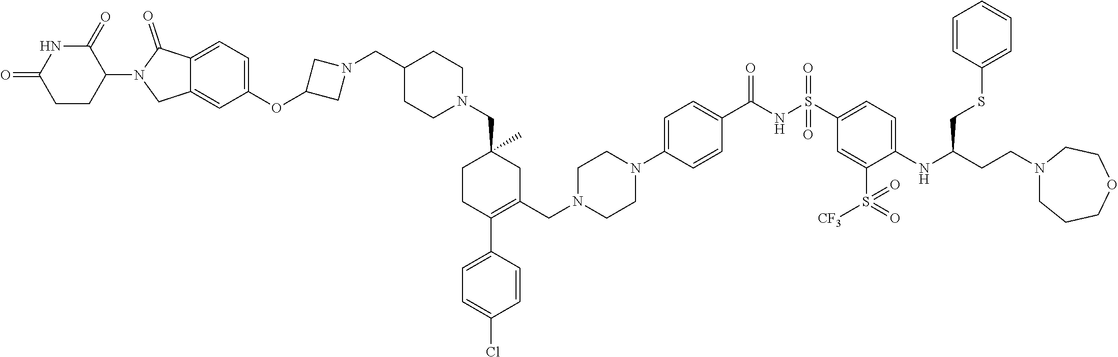 [SMILES: C[C@@]1(CN2CCC(CN3CC(Oc4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 82 | 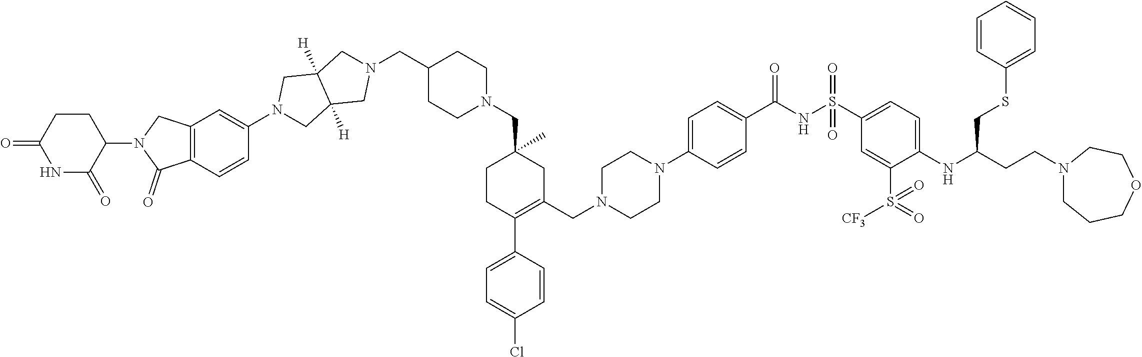 [SMILES: C[C@@]1(CN2CCC(CN3C[C@@H]4CN(c5ccc6c(c5)CN(C5CCC(=O)NC5=O)C6=O)C[C@@H]4C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 83 | 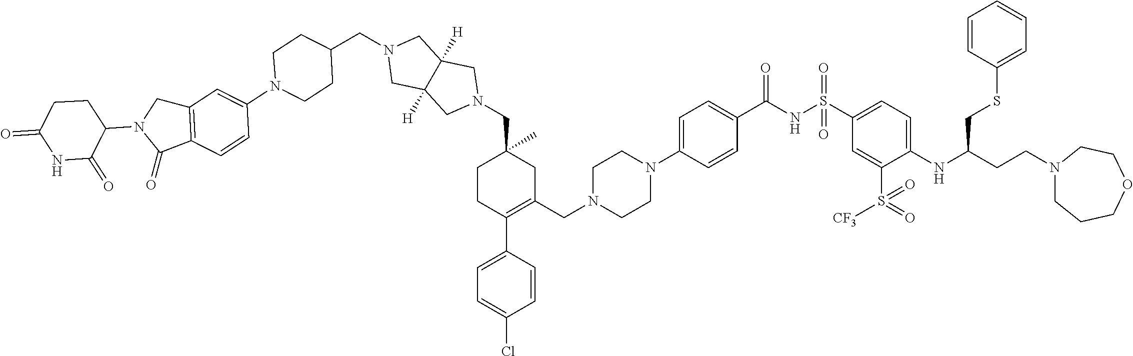 [SMILES: C[C@@]1(CN2C[C@H]3CN(CC4CCN(c5ccc6c(c5)CN(C5CCC(=O)NC5=O)C6=O)CC4)C[C@H]3C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 84 | 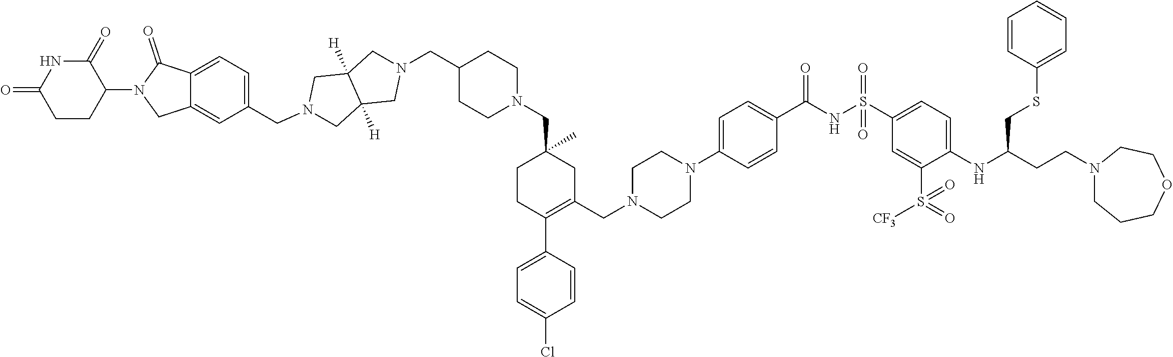 [SMILES: C[C@@]1(CN2CCC(CN3C[C@H]4CN(Cc5ccc6c(c5)CN(C5CCC(=O)NC5=O)C6=O)C[C@H]4C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 85 | 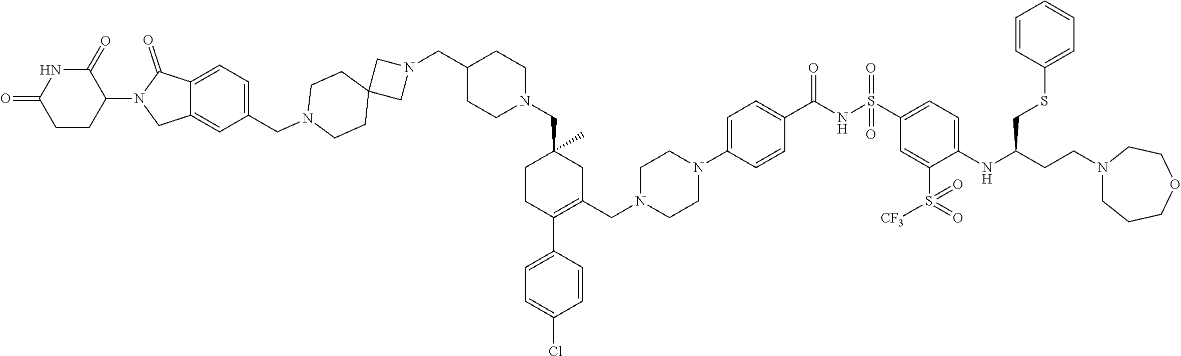 [SMILES: C[C@@]1(CN2CCC(CN3CC4(CCN(Cc5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 86 | 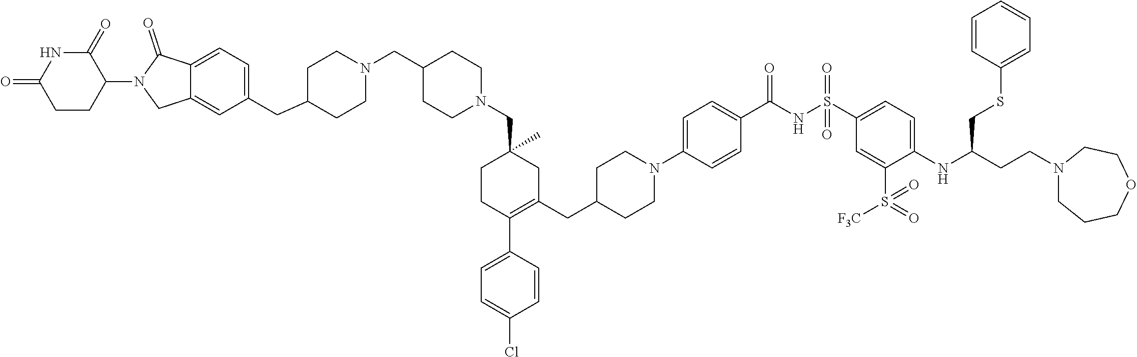 [SMILES: C[C@@]1(CN2CCC(CN3CCC(Cc4ccc5c(c4)CN(C4CCC(=O)NC4=O)C5=O)CC3)CC2)CCC(c2ccc(Cl)cc2)=C(CC2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 87 | 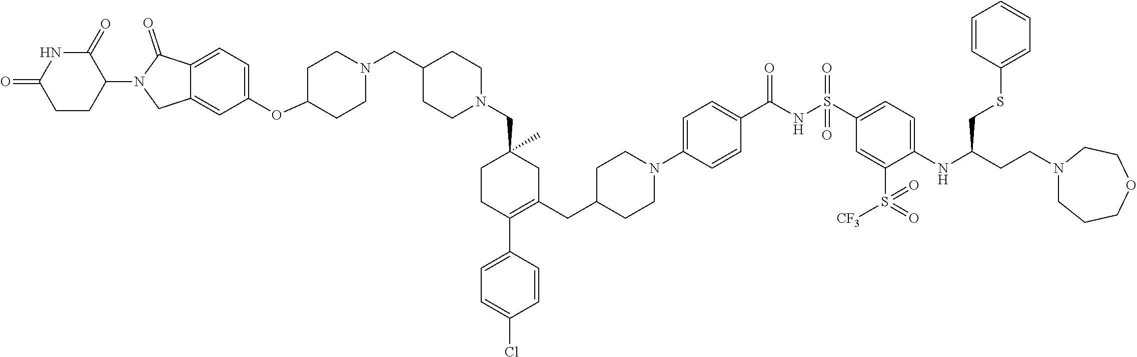 [SMILES: C[C@@]1(CN2CCC(CN3CCC(Oc4ccc5c(c4)CN(C4CCC(=O)NC4=O)C5=O)CC3)CC2)CCC(c2ccc(Cl)cc2)=C(CC2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 88 | 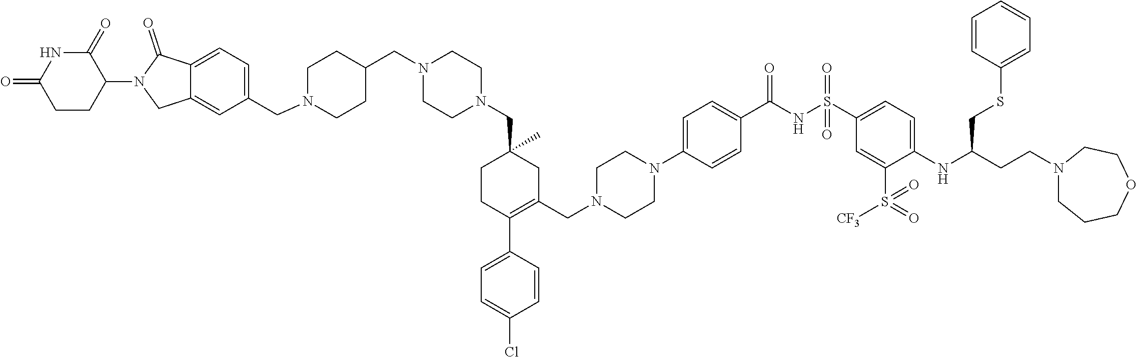 [SMILES: C[C@@]1(CN2CCN(CC3CCN(Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 89 | 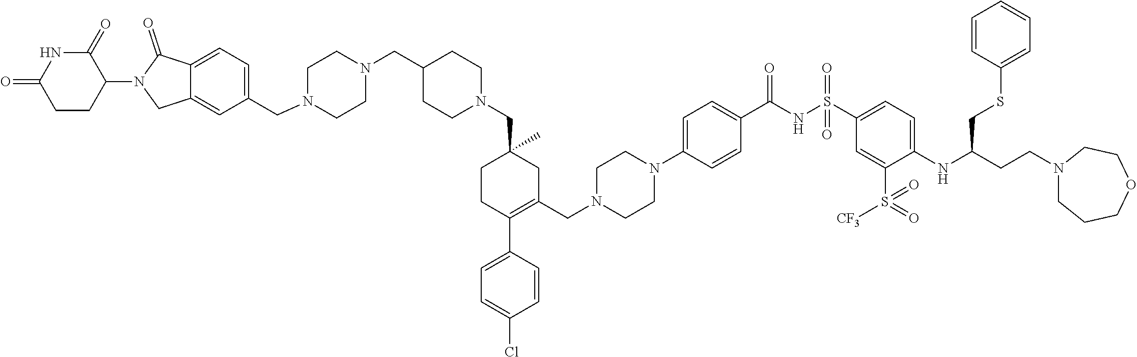 [SMILES: C[C@@]1(CN2CCC(CN3CCN(Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 90 | 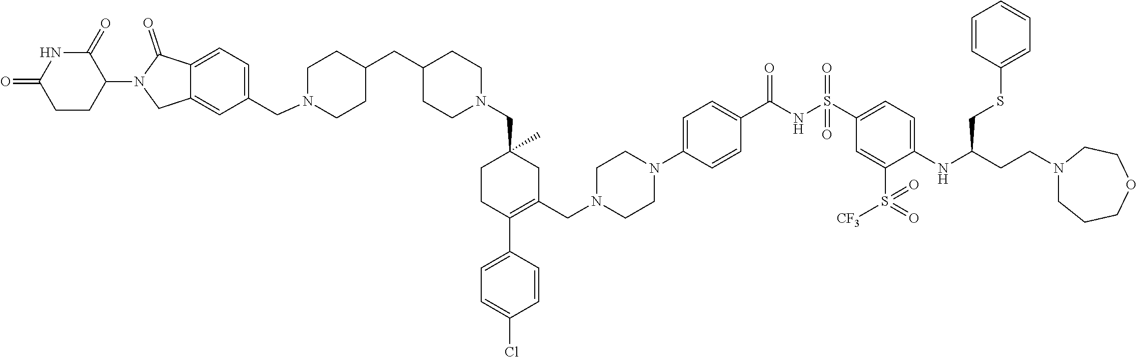 [SMILES: C[C@@]1(CN2CCC(CC3CCN(Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 91 | 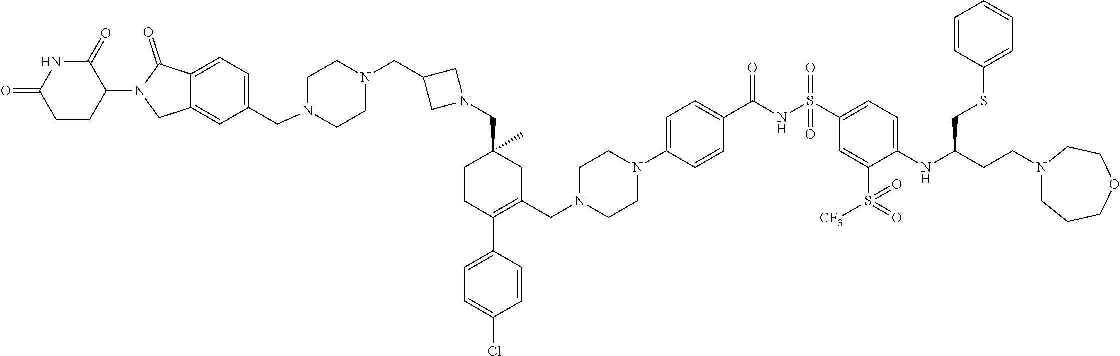 [SMILES: C[C@@]1(CN2CC(CN3CCN(Cc4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 92 | 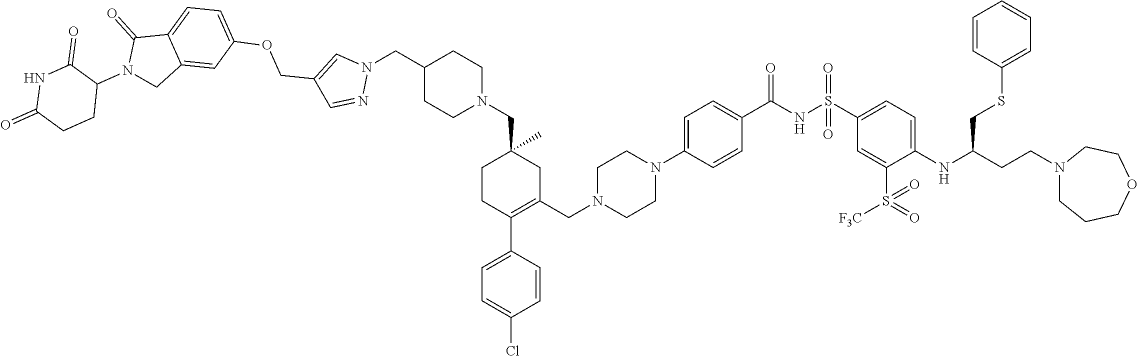 [SMILES: C[C@@]1(CN2CCC(Cn3cc(COc4ccc5c(c4)CN(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 93 | 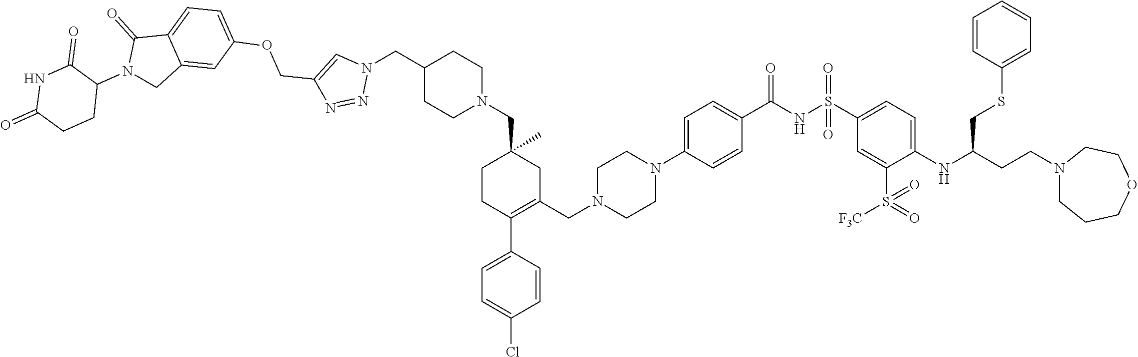 [SMILES: C[C@@]1(CN2CCC(Cn3cc(COc4ccc5c(c4)CN(C4CCC(=O)NC4=O)C5=O)nn3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 94 | 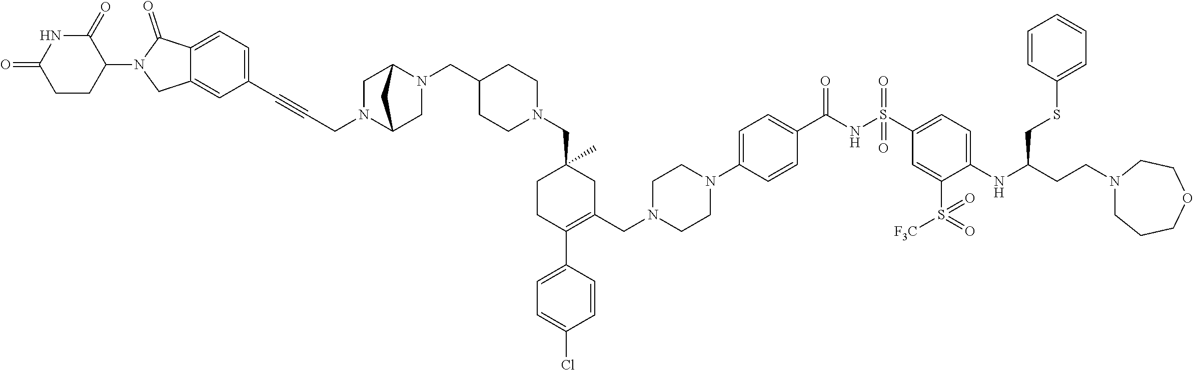 [SMILES: C[C@@]1(CN2CCC(CN3C[C@H]4C[C@@H]3CN4CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 95 | 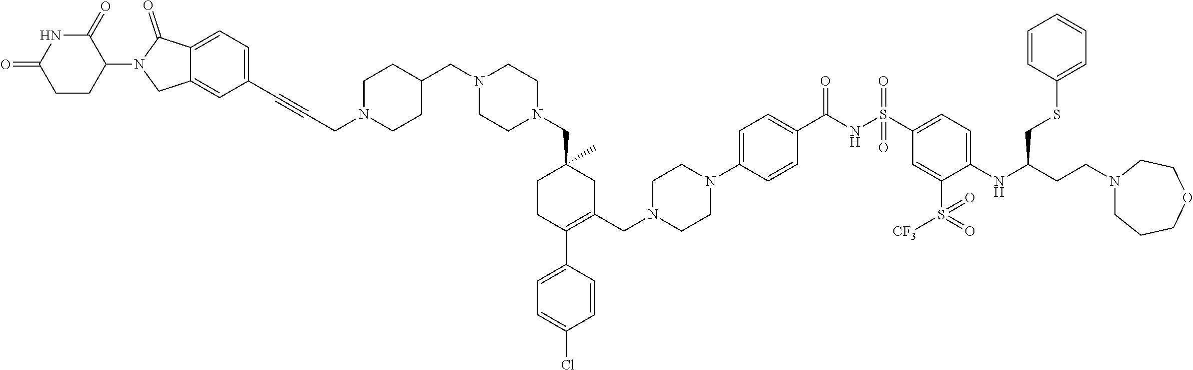 [SMILES: C[C@@]1(CN2CCN(CC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 96 | 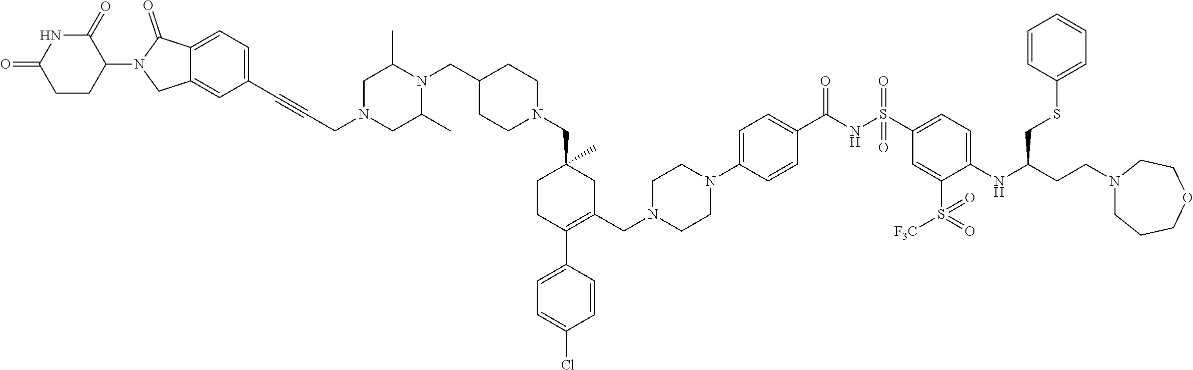 [SMILES: CC1CN(CC#Cc2ccc3c(c2)CN(C2CCC(=O)NC2=O)C3=O)CC(C)N1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 97 | 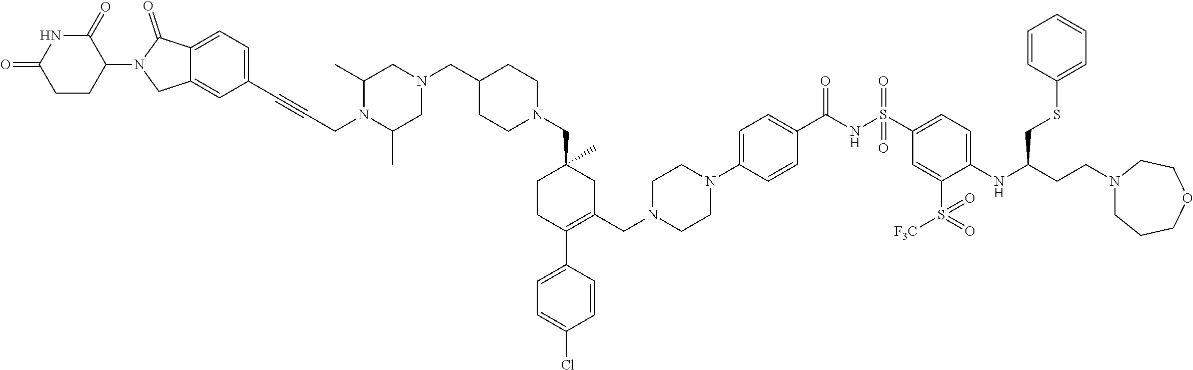 [SMILES: CC1CN(CC2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)CC(C)N1CC#Cc1ccc2c(c1)CN(C1CCC(=O)NC1=O)C2=O] |
| 98 | 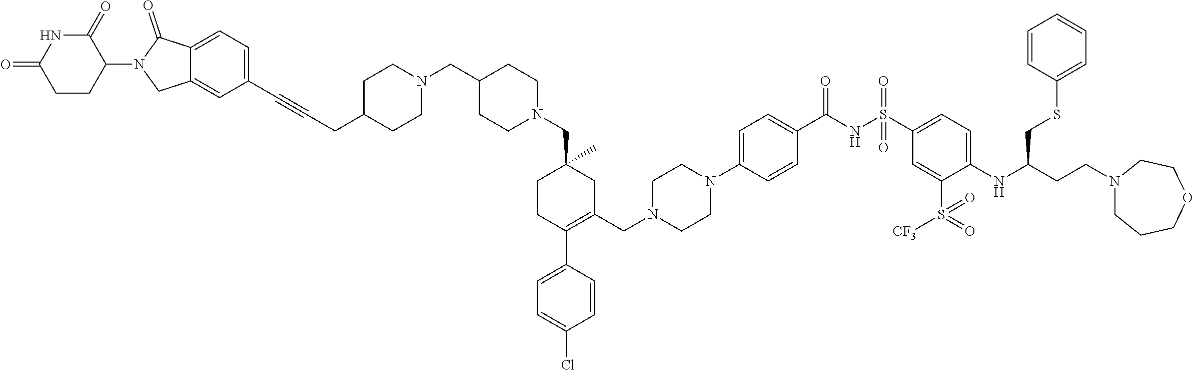 [SMILES: C[C@@]1(CN2CCC(CN3CCC(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 99 | 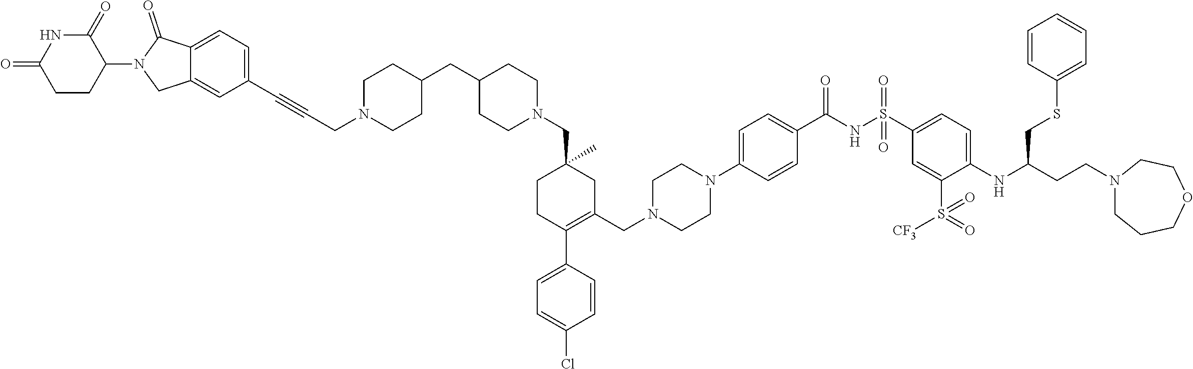 [SMILES: C[C@@]1(CN2CCC(CC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 100 | 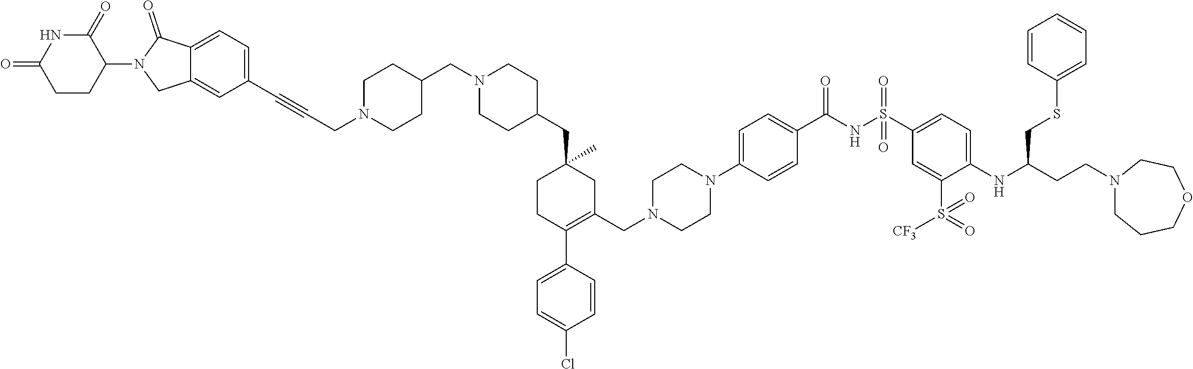 [SMILES: C[C@@]1(CC2CCN(CC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 101 | 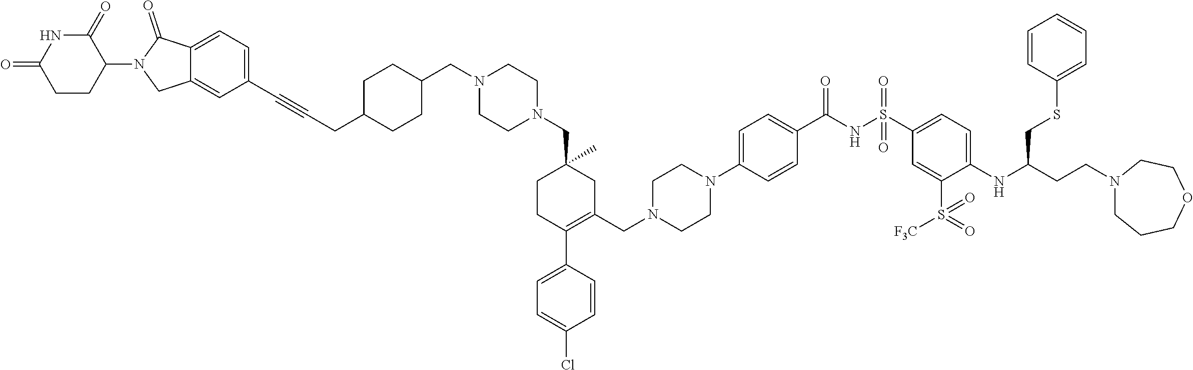 [SMILES: C[C@@]1(CN2CCN(CC3CCC(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 102 | 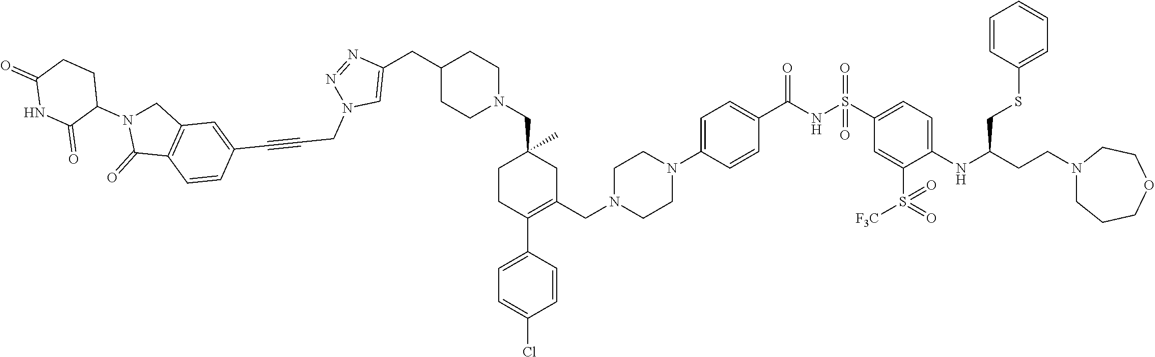 [SMILES: C[C@@]1(CN2CCC(Cc3cn(CC#Cc4ccc5c(c4)CN(C4CCC(=O)NC4=O)C5=O)nn3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 103 | 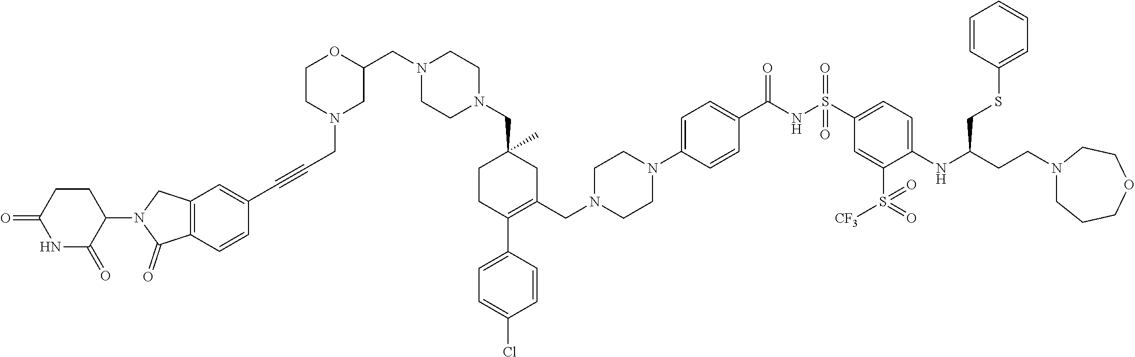 [SMILES: C[C@@]1(CN2CCN(CC3CN(CC#Cc4ccc5c(c4)CN(C4CCC(=O)NC4=O)C5=O)CCO3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 104 | 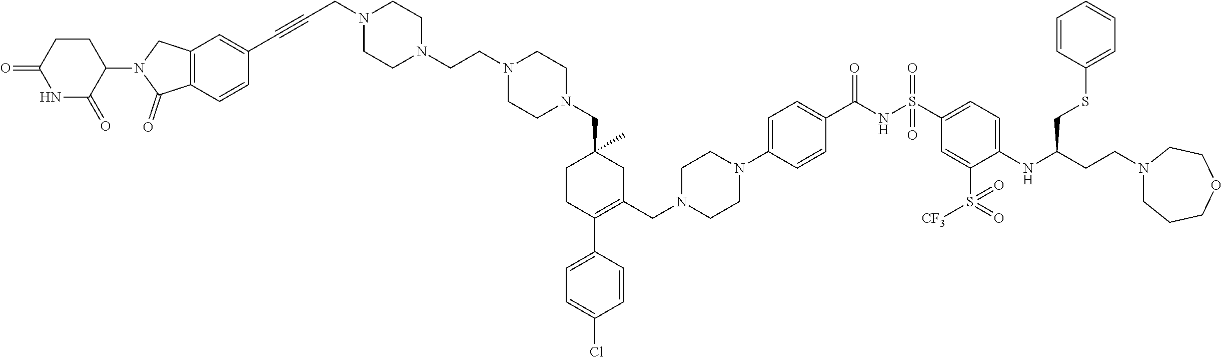 [SMILES: C[C@@]1(CN2CCN(CCN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 105 | 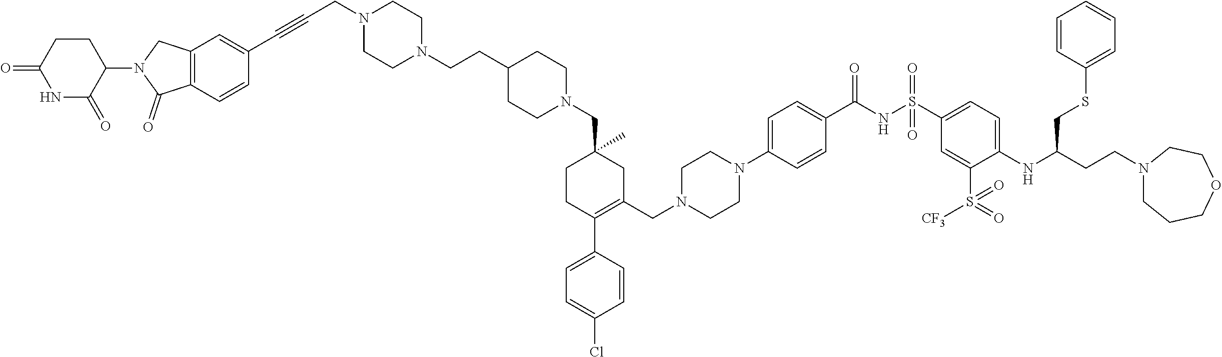 [SMILES: C[C@@]1(CN2CCC(CCN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 106 | 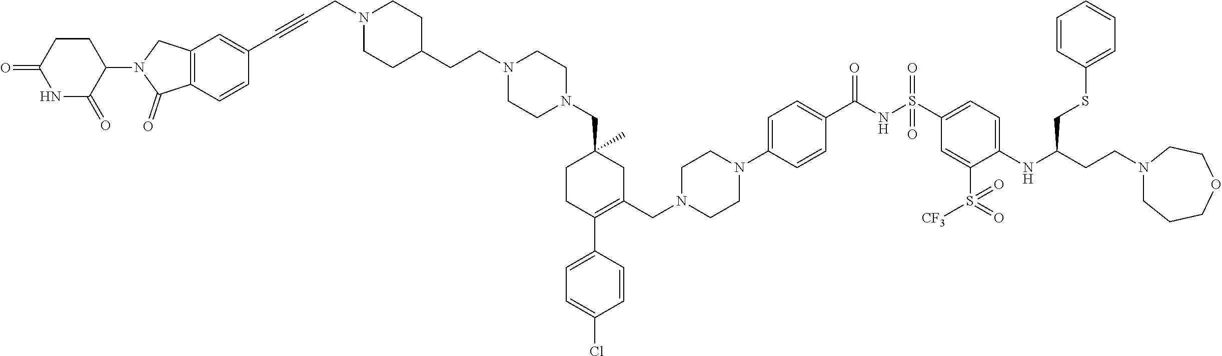 [SMILES: C[C@@]1(CN2CCN(CCC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 107 | 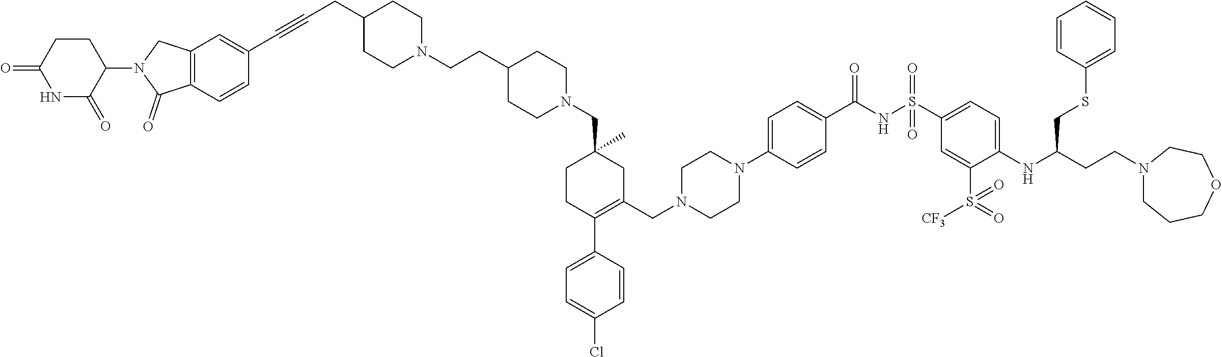 [SMILES: C[C@@]1(CN2CCC(CCN3CCC(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 108 | 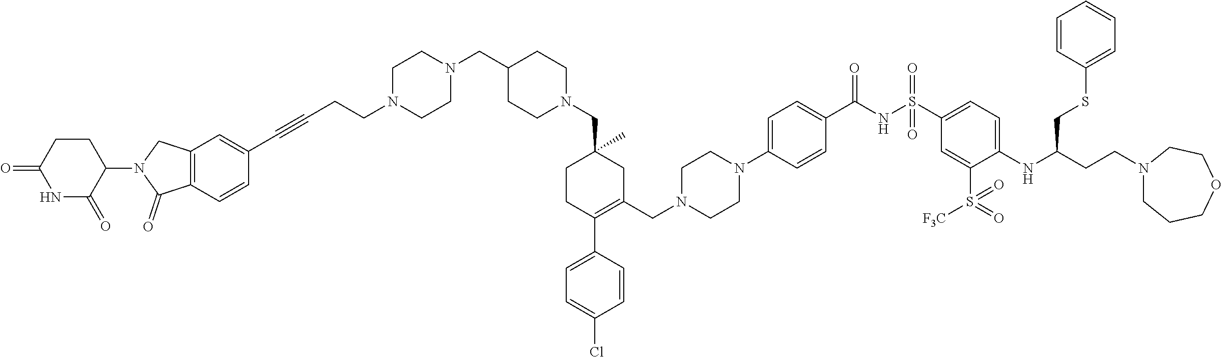 [SMILES: C[C@@]1(CN2CCC(CN3CCN(CCC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 109 | 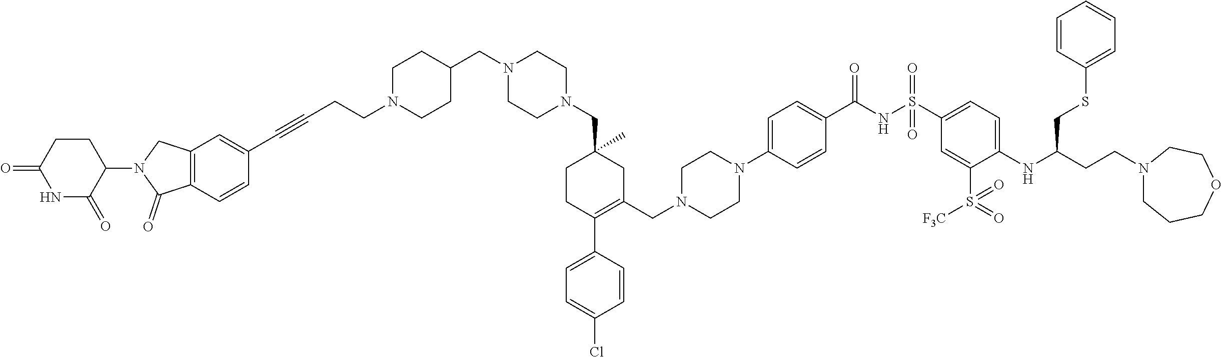 [SMILES: C[C@@]1(CN2CCN(CC3CCN(CCC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 110 | 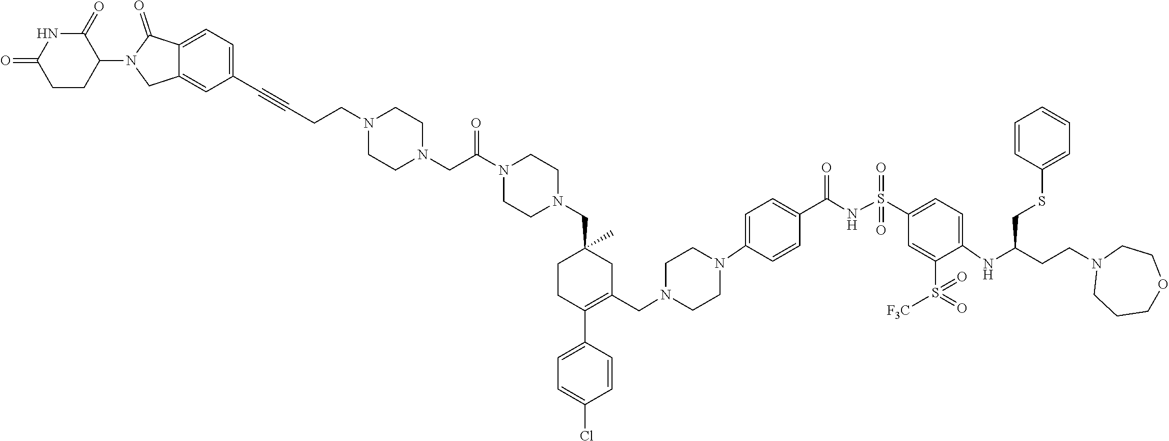 [SMILES: C[C@@]1(CN2CCN(C(=O)CN3CCN(CCC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 111 | 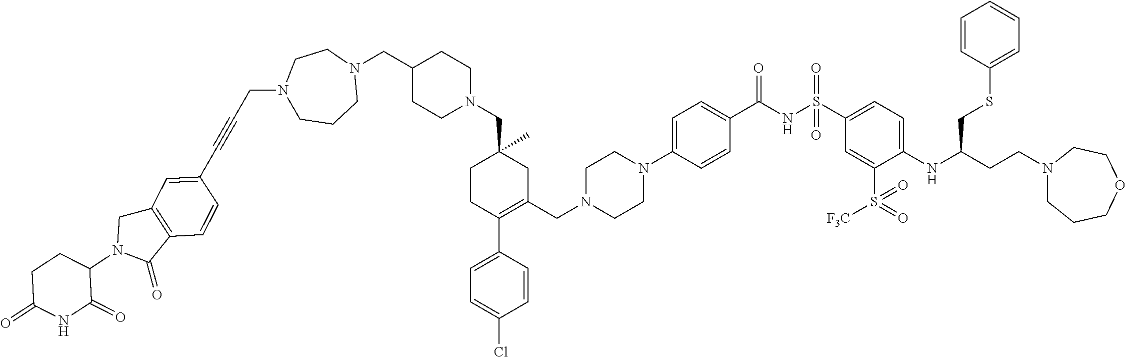 [SMILES: C[C@@]1(CN2CCC(CN3CCCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 112 | 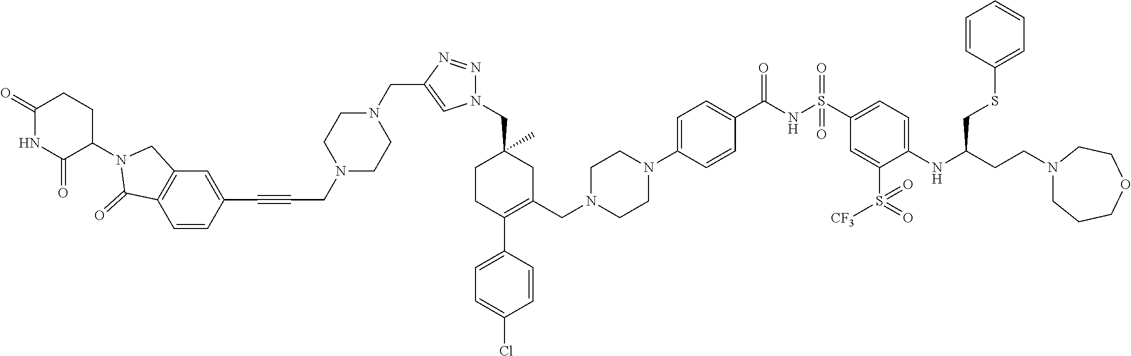 [SMILES: C[C@@]1(Cn2cc(CN3CCN(CC#Cc4ccc5c(c4)CN(C4CCC(=O)NC4=O)C5=O)CC3)nn2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 113 | 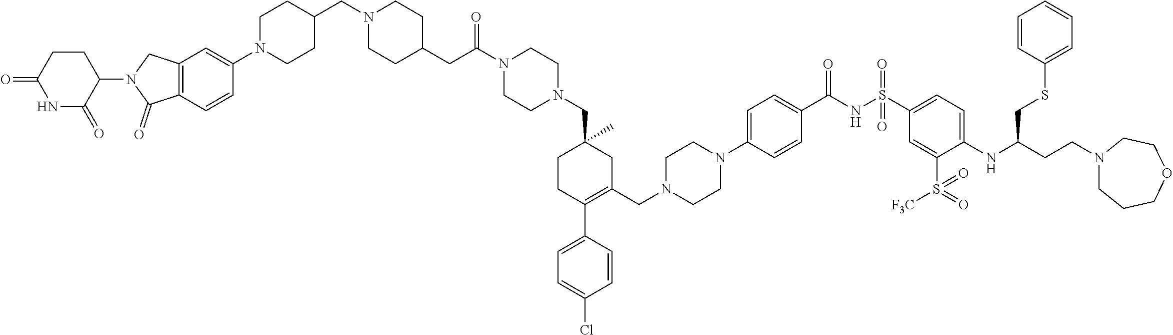 [SMILES: C[C@@]1(CN2CCN(C(=O)CC3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 114 | 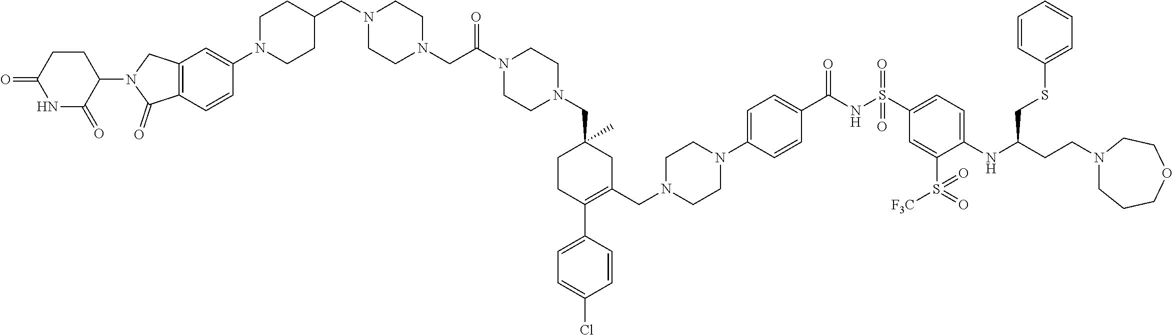 [SMILES: C[C@@]1(CN2CCN(C(=O)CN3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 115 | 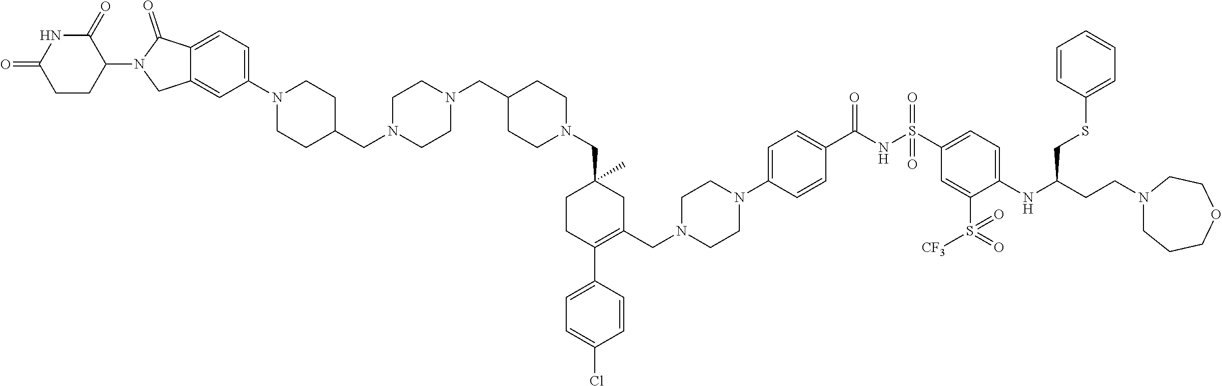 [SMILES: C[C@@]1(CN2CCC(CN3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 116 | 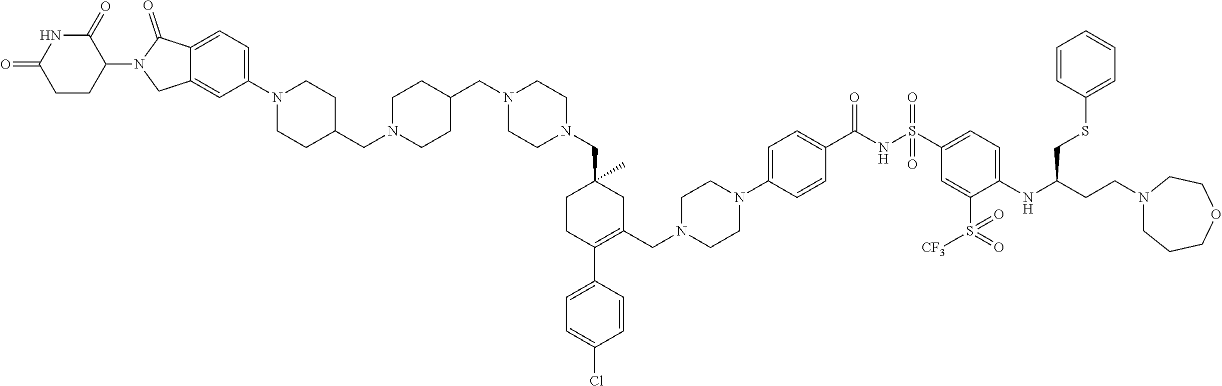 [SMILES: C[C@@]1(CN2CCN(CC3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 117 | 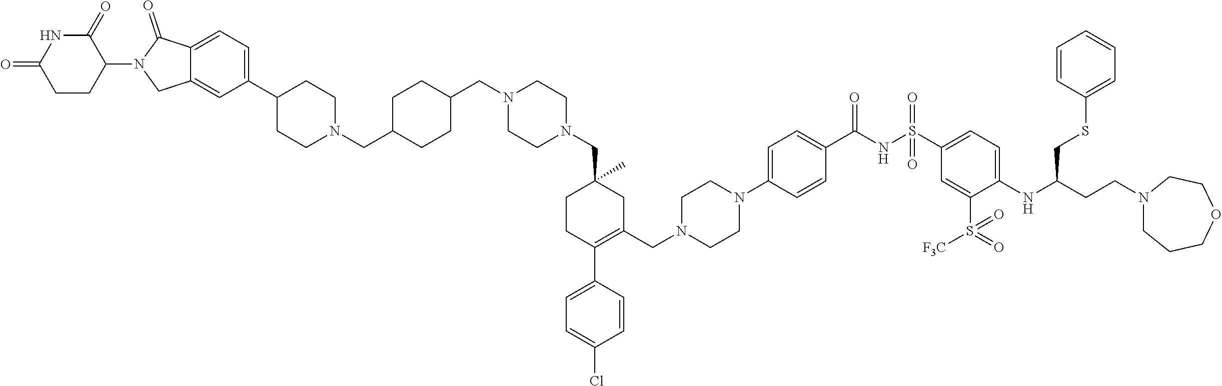 [SMILES: C[C@@]1(CN2CCN(CC3CCC(CN4CCC(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 118 | 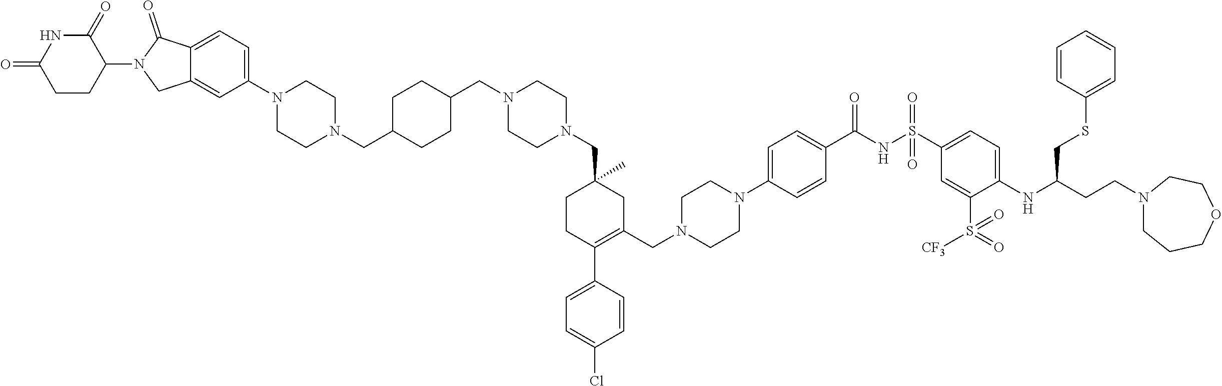 [SMILES: C[C@@]1(CN2CCN(CC3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 119 | 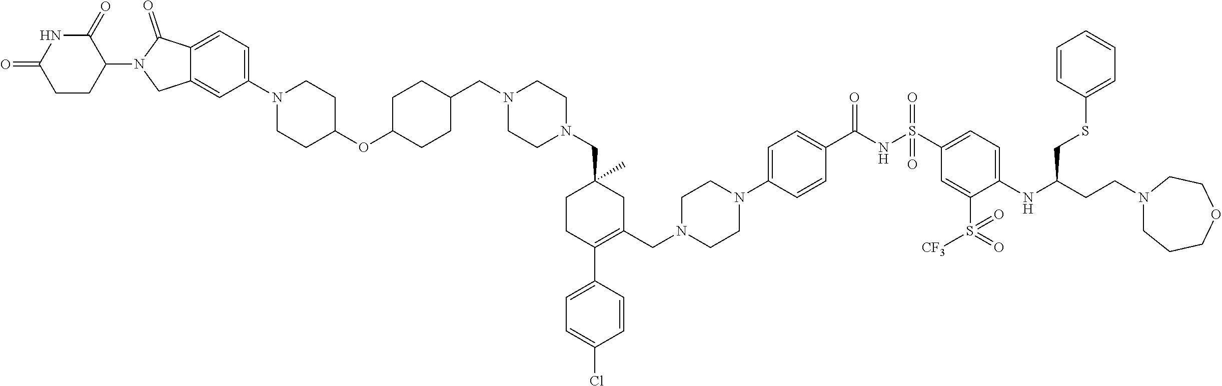 [SMILES: C[C@@]1(CN2CCN(CC3CCC(OC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 120 | 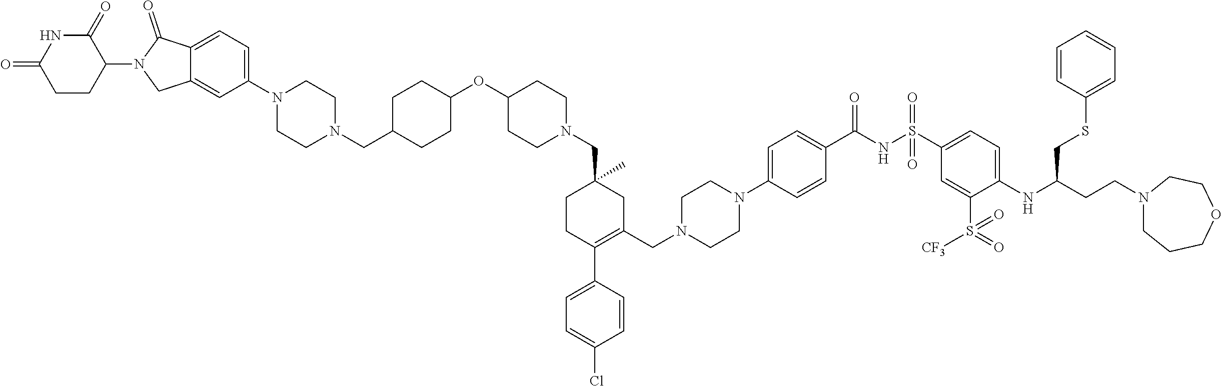 [SMILES: C[C@@]1(CN2CCC(OC3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 121 | 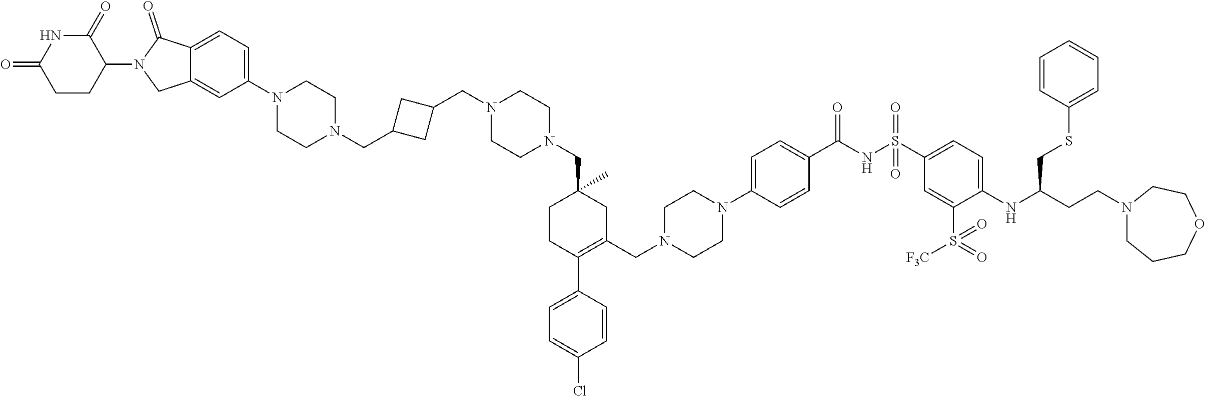 [SMILES: C[C@@]1(CN2CCN(CC3CC(CN4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 122 | 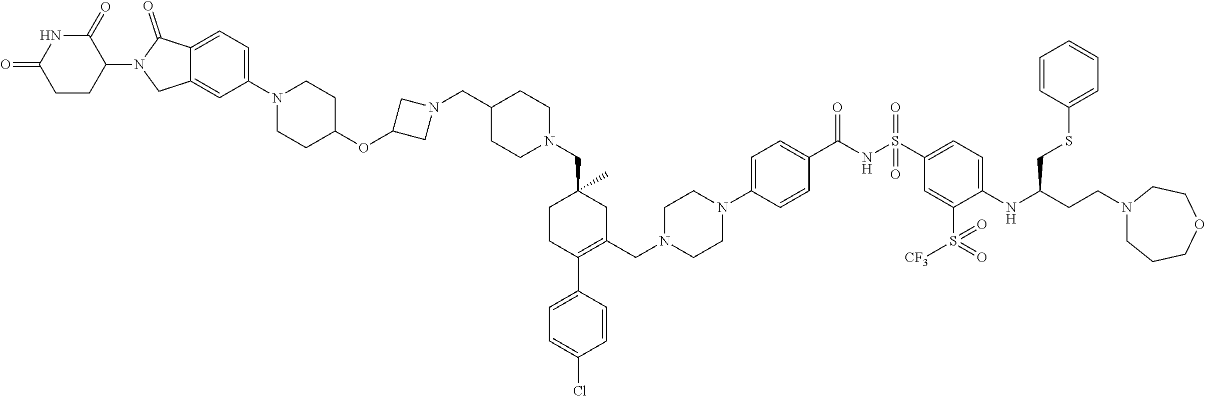 [SMILES: C[C@@]1(CN2CCC(CN3CC(OC4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 123 | 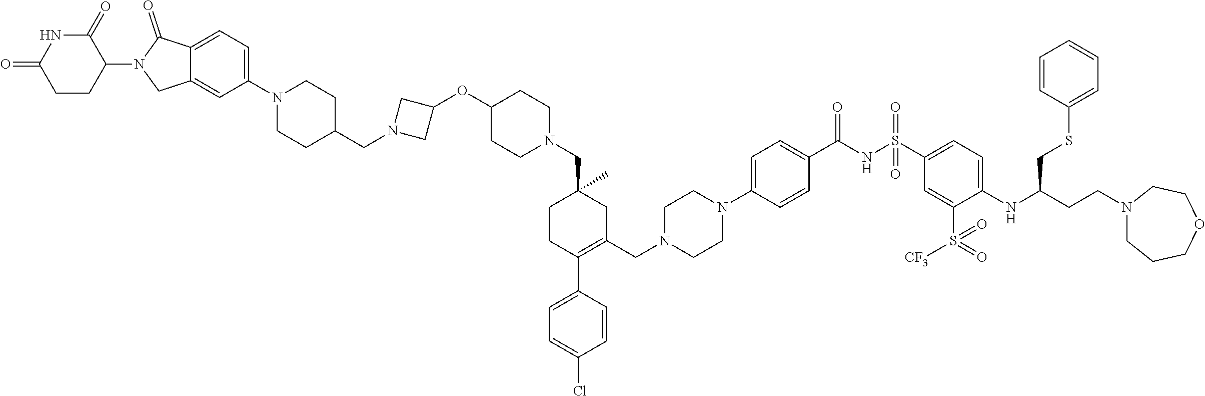 [SMILES: C[C@@]1(CN2CCC(OC3CN(CC4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 124 | 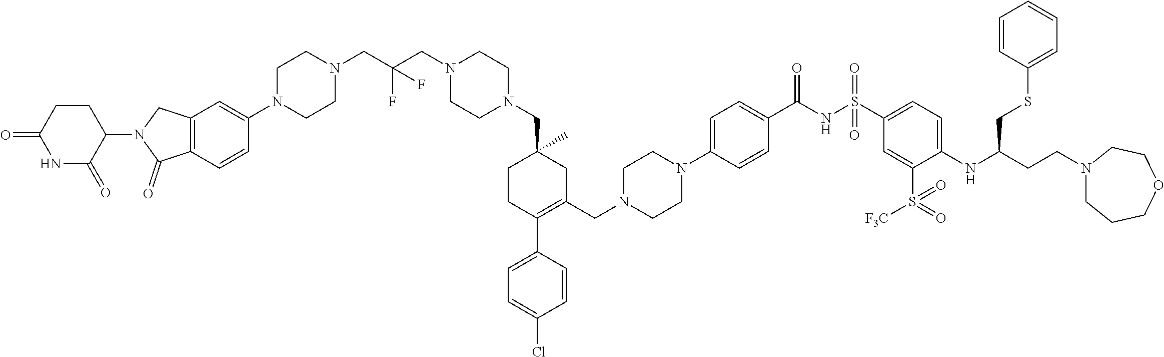 [SMILES: C[C@@]1(CN2CCN(CC(F)(F)CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 125 | 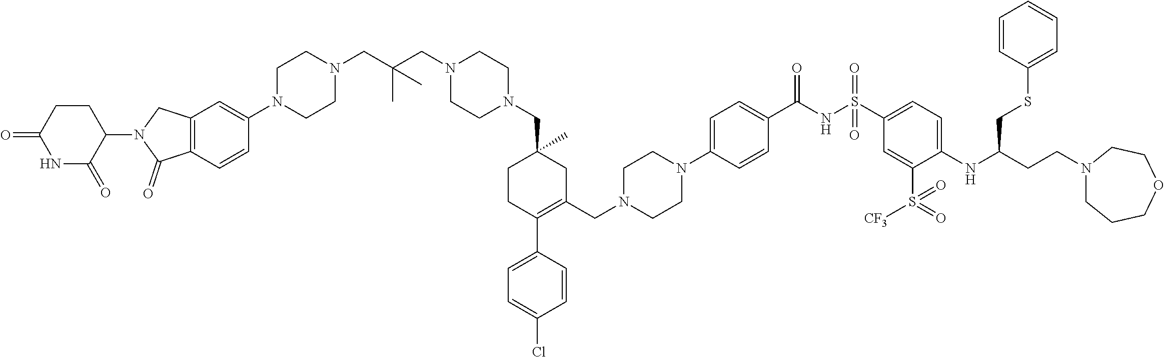 [SMILES: CC(C)(CN1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1)CN1CCN(c2ccc3c(c2)CN(C2CCC(=O)NC2=O)C3=O)CC1] |
| 126 | 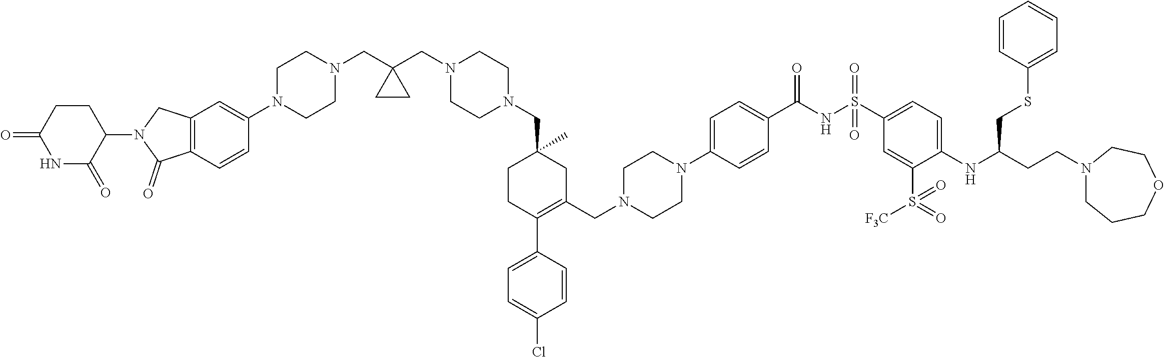 [SMILES: C[C@@]1(CN2CCN(CC3(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 127 | 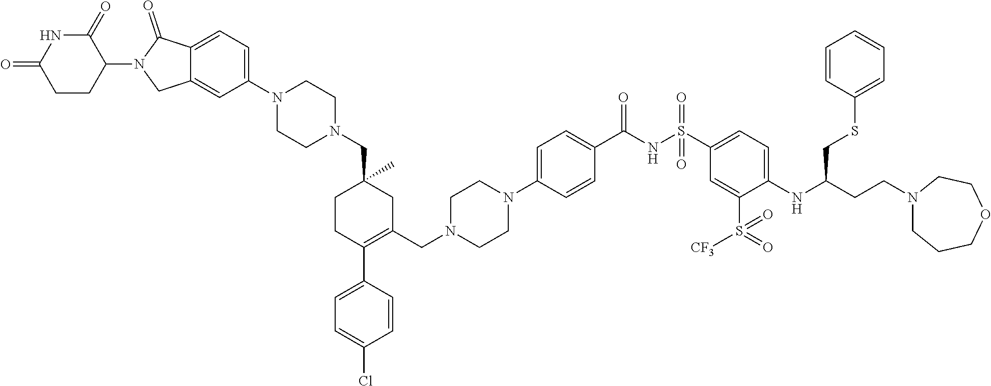 [SMILES: C[C@@]1(CN2CCN(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 128 | 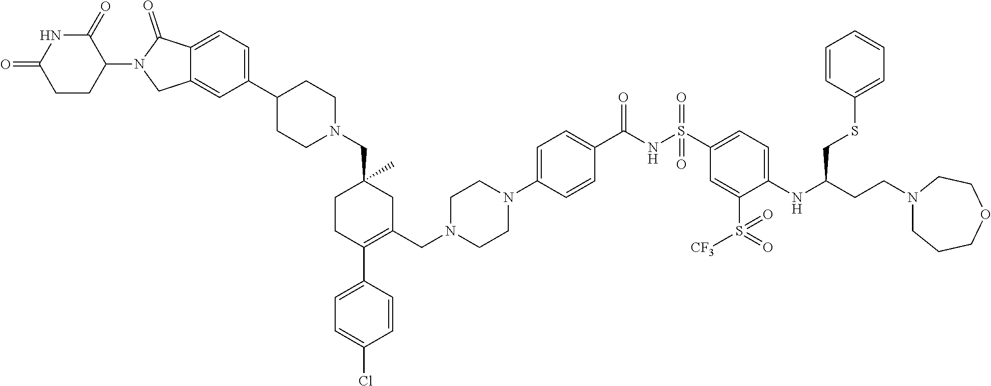 [SMILES: C[C@@]1(CN2CCC(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |

or a pharmaceutically acceptable salt, solvate, hydrate, polymorph, co-crystal, tautomer, stereoisomer, isotopically labeled compound, or prodrug thereof.

In certain embodiments, the compound is of the formula:

| Com- | |
|---|---|
| pound | |
| Number | Formula |
| 129 | 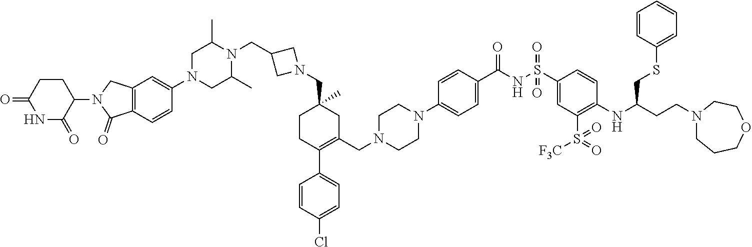 [SMILES: CC1CN(c2ccc3c(c2)CN(C2CCC(=O)NC2=O)C3=O)CC(C)N1CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)C1] |
| 130 | 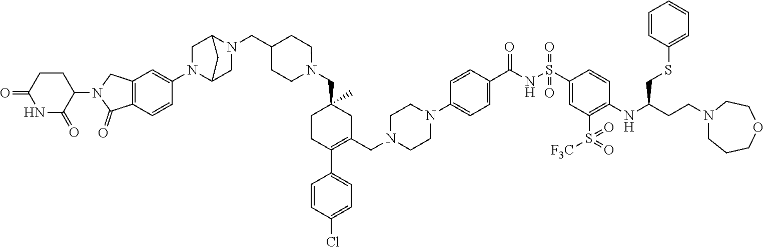 [SMILES: C[C@@]1(CN2CCC(CN3CC4CC3CN4c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 131 | 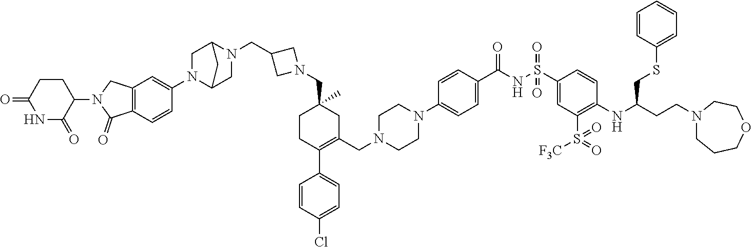 [SMILES: C[C@@]1(CN2CC(CN3CC4CC3CN4c3ccc4c(c3)CN(C3CCC(=O)NC3=O)C4=O)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 132 | 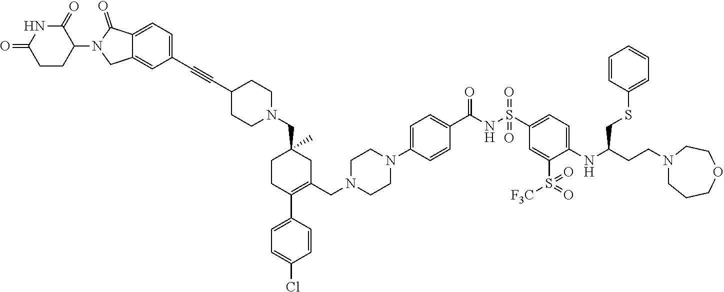 [SMILES: C[C@@]1(CN2CCC(C#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 133 | 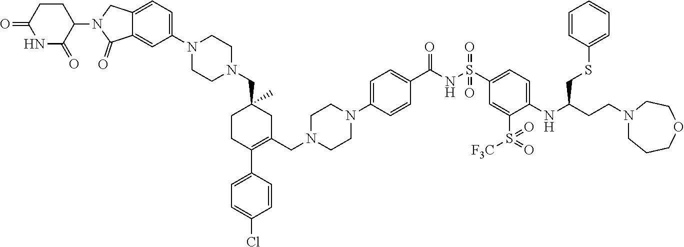 [SMILES: C[C@@]1(CN2CCN(c3ccc4c(c3)C(=O)N(C3CCC(=O)NC3=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 134 | 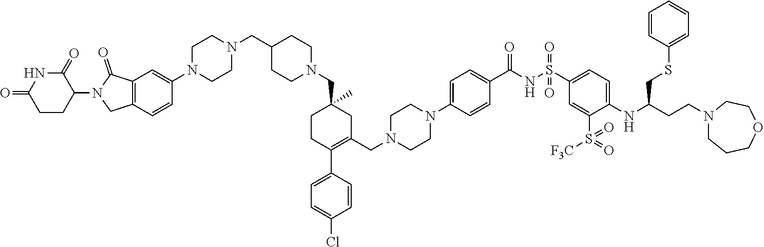 [SMILES: C[C@@]1(CN2CCC(CN3CCN(c4ccc5c(c4)C(=O)N(C4CCC(=O)NC4=O)C5)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 135 | 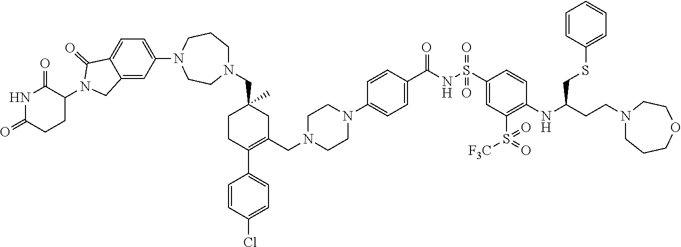 [SMILES: C[C@@]1(CN2CCCN(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 136 | 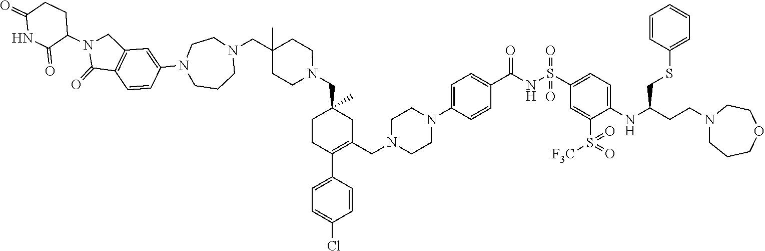 [SMILES: CC1(CN2CCCN(c3ccc4c(c3)CN(C3CCC(=O)NC3=O)C4=O)CC2)CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 137 | 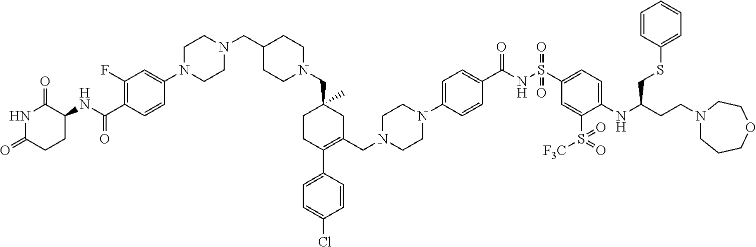 [SMILES: C[C@@]1(CN2CCC(CN3CCN(c4ccc(C(=O)N[C@H]5CCC(=O)NC5=O)c(F)c4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 138 | 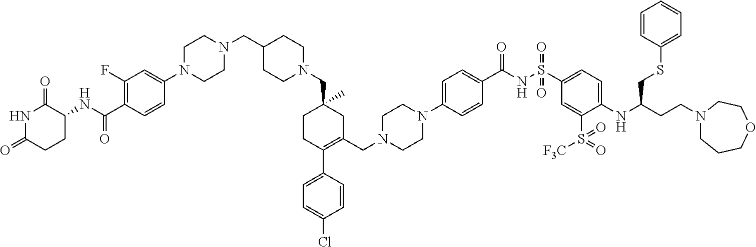 [SMILES: C[C@@]1(CN2CCC(CN3CCN(c4ccc(C(=O)N[C@@H]5CCC(=O)NC5=O)c(F)c4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 139 | 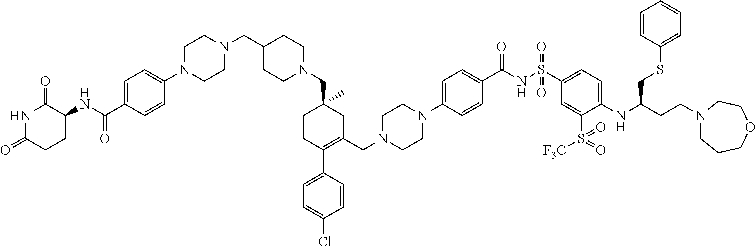 [SMILES: C[C@@]1(CN2CCC(CN3CCN(c4ccc(C(=O)N[C@H]5CCC(=O)NC5=O)cc4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 140 | 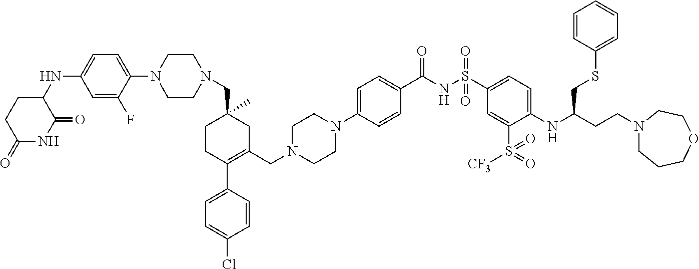 [SMILES: C[C@@]1(CN2CCN(c3ccc(NC4CCC(=O)NC4=O)cc3F)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 141 | 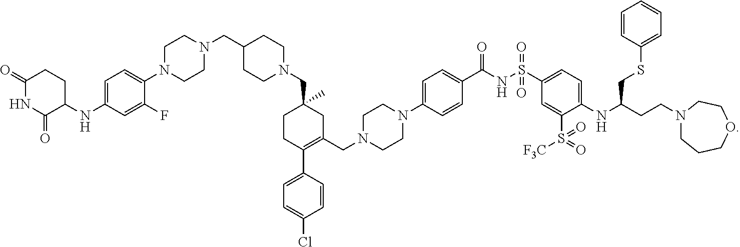 [SMILES: C[C@@]1(CN2CCC(CN3CCN(c4ccc(NC5CCC(=O)NC5=O)cc4F)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |

or a pharmaceutically acceptable salt, solvate, hydrate, polymorph, co-crystal, tautomer, stereoisomer, isotopically labeled compound, or prodrug thereof.

In certain embodiments, a provided compound (a compound of the present disclosure, a compound provided herein) is a compound of Formula (I), or a pharmaceutically acceptable salt, solvate, hydrate, polymorph, co-crystal, tautomer, stereoisomer, isotopically labeled compound, or prodrug thereof. In certain embodiments, a provided compound is a compound of Formula (I), or a pharmaceutically acceptable salt, solvate, hydrate, polymorph, co-crystal, tautomer, stereoisomer, or isotopically labeled compound thereof. In certain embodiments, a provided compound is a compound of Formula (I), or a pharmaceutically acceptable salt, tautomer, or stereoisomer thereof. In certain embodiments, a provided compound is a compound of Formula (I), or a pharmaceutically acceptable salt thereof. In certain embodiments, a provided compound is a mixture (e.g., a racemic mixture) of stereoisomers. In certain embodiments, a provided compound is any one of Compounds 1 to 20, or a pharmaceutically acceptable salt, solvate, hydrate, polymorph, co-crystal, tautomer, stereoisomer, isotopically labeled compound, or prodrug thereof. In certain embodiments, a provided compound is any one of Compounds to 20, or a pharmaceutically acceptable salt thereof.

Compounds provided herein can be obtained from natural sources or made or modified made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43:406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database. For example, compounds of formulae herein can be made using methodology known in the art, including Additionally, general principles of organic chemistry, as well as specific functional moieties and reactivity, are described in Thomas Sorrell, Organic Chemistry, University Science Books, Sausalito, 1999;Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

The compounds provided herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present disclosure. The compounds herein may also be represented in multiple tautomeric forms, in such instances, the disclosure expressly includes all tautomeric forms of the compounds provided herein, even though only a single tautomeric form may be represented. All such isomeric forms of such compounds herein are expressly included in the present disclosure. All crystal forms and polymorphs of the compounds provided herein are expressly included in the present disclosure. All hydrate and solvate forms of the compounds provided herein are expressly included in the present disclosure. Also embodied are extracts and fractions comprising compounds provided herein. The term “isomers” is intended to include diastereoisomers, enantiomers, regioisomers, structural isomers, rotational isomers, tautomers, and the like. All such isomers of such compounds herein are expressly included in the present disclosure. For compounds which contain one or more stereogenic centers, e.g., chiral compounds, the methods provided herein may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound the present disclosure provides administered to cells or a subject.

The compounds of the formulae herein can be synthesized using methodology similarly to that described in Chen, Q. Y.; Liu, Y.; Cai, W.; Luesch, H. Improved Total Synthesis and Biological Evaluation of Potent Apratoxin S4 Based Anticancer Agents with Differential Stability and Further Enhanced Activity. J. Med. Chem. 2014, 57 (7): p. 3011-302; and in WO2012/158933.

The present disclosure provides compounds which are in a salt form. In some embodiments, the salt is a pharmaceutically acceptable salt. Certain specific compounds provided herein contain both basic and acidic functionalities that allow the compounds to be converted into either base or acid addition salts. The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, provided herein are compounds which are in a prodrug form. Prodrugs of the compounds provided herein are those compounds that readily undergo chemical changes under physiological conditions to provide the compounds provided herein. Additionally, prodrugs can be converted to the compounds provided herein by chemical or biochemical methods in an ex vivo environment. For example, prodrugs can be slowly converted to the compounds provided herein when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are intended to be encompassed within the scope of the present disclosure. Certain compounds provided herein may exist in multiple crystalline or amorphous forms. In general, all physical forms are equivalent for the uses contemplated by the present disclosure and are intended to be within the scope of the present disclosure.

In certain embodiments, the provided compounds bind to Bcl-2 with a dissociation constant (K_{d}) of less than 1 pM, between 1 and 10 pM, between 10 and 100 pM, between 0.1 and 1 nM, between 1 and 10 nM, between 10 and 100 nM, between 0.1 and 1 μM, between 1 and 10 μM, between 10 and 30 μM, or between 30 and 100 μM.

In certain embodiments, the provided compounds inhibit the activity of Bcl-2 with an IC₅₀ of less than 1 pM, between 1 and 10 pM, between 10 and 100 pM, between 0.1 and 1 nM, between 1 and 10 nM, between 10 and 100 nM, between 0.1 and 1 μM, between 1 and 10 μM, between 10 and 30 μM, or between 30 and 100 μM.

In certain embodiments, the provided compounds inhibit the production of Bcl-2 with an IC₅₀ of less than 1 pM, between 1 and 10 pM, between 10 and 100 pM, between 0.1 and 1 nM, between 1 and 10 nM, between 10 and 100 nM, between 0.1 and 1 μM, between 1 and 10 μM, between 10 and 30 μM, or between 30 and 100 μM.

In certain embodiments, the provided compounds bind to Bcl-xL with a dissociation constant (K_{d}) of less than 1 μM, between 1 and 10 μM, between 10 and 100 pM, between 0.1 and 1 nM, between 1 and 10 nM, between 10 and 100 nM, between 0.1 and 1 μM, between 1 and 10 μM, between 10 and 30 μM, or between 30 and 100 μM.

In certain embodiments, the provided compounds inhibit the activity of Bcl-xL with an IC₅₀ of less than 1 pM, between 1 and 10 pM, between 10 and 100 pM, between 0.1 and 1 nM, between 1 and 10 nM, between 10 and 100 nM, between 0.1 and 1 μM, between 1 and 10 μM, between 10 and 30 μM, or between 30 and 100 μM.

In certain embodiments, the provided compounds inhibit the production of Bcl-xL with an IC₅₀ of less than 1 pM, between 1 and 10 pM, between 10 and 100 pM, between 0.1 and 1 nM, between 1 and 10 nM, between 10 and 100 nM, between 0.1 and 1 μM, between 1 and 10 μM, between 10 and 30 μM, or between 30 and 100 μM.

In certain embodiments, the provided compounds are able to bind to both Bcl-2 and Bcl-xL. In certain embodiments, the provided compounds inhibit the activity of both Bcl-2 and Bcl-xL. In certain embodiments, the provided compounds inhibit the production of both Bcl-2 and Bcl-xL.

In certain embodiments, the selectivity (e.g., as determined by K_{d} or IC₅₀) of the provided compounds for Bcl-2 over a different Bcl-2 family protein (e.g., Bcl-xL) is between 2 and 3, between 3 and 5, between 5 and 10, between 10 and 30, between 30 and 100, between 100 and 300, or between 300 and 1000 folds, or greater than 1000 folds.

In certain embodiments, the selectivity (e.g., as determined by K_{d} or IC₅₀) of the provided compounds for Bcl-xL over a different Bcl-2 family protein (e.g., Bcl-2) is between 2 and 3, between 3 and 5, between 5 and 10, between 10 and 30, between 30 and 100, between 100 and 300, or between 300 and 1000 folds, or greater than 1000 folds.

In certain embodiments, a compound provided herein has reduced platelet toxicity relative to other Bcl-2 and/or Bcl-xL inhibitors. In certain embodiments, the ratio of human platelet toxicity (IC₅₀) to anticancer activity (IC₅₀) of the compound the present disclosure provides less than that of other Bcl-2 and/or Bcl-xL inhibitors (e.g., the another Bcl-2 inhibitor and the another Bcl-xL inhibitor described herein).

The provided compounds may be prepared by methods known in the art, e.g., the methods described in U.S. Patent Application Publication No. 2022-0169628.

### Pharmaceutical Compositions, Kits, and Administration

In another aspect, the present disclosure provides a pharmaceutical composition comprising a compound of the present disclosure, and a pharmaceutically acceptable carrier.

In certain embodiments, the pharmaceutical composition comprises an effective amount of the provided compound. In certain embodiments, the effective amount is a therapeutically effective amount. In certain embodiments, the effective amount is a prophylactically effective amount. In certain embodiments, the effective amount is an amount effective for treating a disease in a subject in need thereof. In certain embodiments, the effective amount is an amount effective for preventing a disease in a subject in need thereof. In certain embodiments, the effective amount is an amount effective for reducing the risk of developing a disease in a subject in need thereof. In certain embodiments, the effective amount is an amount effective for binding Bcl-xL and/or Bcl-2 (e.g., in a subject, tissue, biological sample, or cell). In certain embodiments, the effective amount is an amount effective for inhibiting the production of Bcl-xL and/or Bcl-2 (e.g., in a subject, tissue, biological sample, or cell). In certain embodiments, the effective amount is an amount effective for inhibiting the activity (e.g., aberrant activity, such as increased activity) of Bcl-xL and/or Bcl-2 (e.g., in a subject, tissue, biological sample, or cell).

In certain embodiments, the effective amount is an amount effective for binding Bcl-xL and/or Bcl-2 by between 0.1% and 10%, between 10% and 20%, between 20% and 30%, between 30% and 40%, between 40% and 50%, between 50% and 60%, between 60% and 70%, between 70% and 80%, between 80% and 90%, between 90% and 95%, between 95% and 98%, between 98% and 99%, or greater than 99%.

In certain embodiments, the effective amount is an amount effective for inhibiting the production of Bcl-xL and/or Bcl-2 by between 0.1% and 10%, between 10% and 20%, between 20% and 30%, between 30% and 40%, between 40% and 50%, between 50% and 60%, between 60% and 70%, between 70% and 80%, between 80% and 90%, between 90% and 95%, between 95% and 98%, between 98% and 99%, or greater than 99%.

In certain embodiments, the effective amount is an amount effective for inhibiting the activity of Bcl-xL and/or Bcl-2 by between 0.1% and 10%, between 10% and 20%, between 20% and 30%, between 30% and 40%, between 40% and 50%, between 50% and 60%, between 60% and 70%, between 70% and 80%, between 80% and 90%, between 90% and 95%, between 95% and 98%, between 98% and 99%, or greater than 99%.

In certain embodiments, the pharmaceutical composition is for use in treating a disease. In certain embodiments, the pharmaceutical composition is for use in preventing a disease. In certain embodiments, the pharmaceutical composition is for use in inhibiting the activity of Bcl-xL and/or Bcl-2. In certain embodiments, the pharmaceutical composition is for use in inhibiting the production of Bcl-xL and/or Bcl-2. In certain embodiments, the pharmaceutical composition is for use in inhibiting the activity of Bcl-xL and/or Bcl-2.

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk of developing a disease in a subject in need thereof), improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease.

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, cytotoxic agents, anti-angiogenesis agents, anti-inflammatory agents, and immunosuppressants. In certain embodiments, the additional pharmaceutical agent is an anti-inflammatory agent. In certain embodiments, the additional pharmaceutical agent is an immunotherapy. In certain embodiments, the additional pharmaceutical agent is an anti-proliferative agent. In certain embodiments, the additional pharmaceutical agent is an anti-cancer agent. In certain embodiments, the anti-cancer agents include, but are not limited to, epigenetic or transcriptional modulators (e.g., DNA methyltransferase inhibitors, HDAC inhibitors, lysine methyltransferase inhibitors), antimitotic drugs (e.g., taxanes and vinca alkaloids), cell signaling pathway inhibitors (e.g., tyrosine protein kinase inhibitors), modulators of protein stability (e.g., proteasome inhibitors), Hsp90 inhibitors, glucocorticoids, all-trans retinoic acids, anti-estrogens (e.g., tamoxifen, raloxifene, and megestrol), LHRH agonists (e.g., goscrclin and leuprolide), anti-androgens (e.g. flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca2⁺ ATPase inhibitors (e.g., thapsigargin), thalidomide, lenalidomide, pomalidomide, tyrosine kinase inhibitors (e.g., axitinib, bosutinib, cediranib (RECENTIN™), dasatinib (SPRYCEL®), erlotinib (TARCEVA®), gefitinib (IRESSA®), imatinib (Gleevec®), lapatinib (TYKERB®, TYVERB®), lestaurtinib, neratinib, nilotinib (TASIGNA®), semaxanib, sunitinib (SUTENT®), toceranib (PALLADIA®), vandetanib (ZACTIMA®, ZD6474), vatalanib (PTK787), nilotinib (TASIGNA®), sorafenib (NEXAVAR®), everolimus (AFINITOR®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the anti-cancer agent is alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Suitable anti-metabolites include, but are not limited to aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcetabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Non-limiting examples of suitable anti-tumor antibiotics include aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin.

Non-limiting examples of suitable anti-cytoskeletal agents include cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine.

Suitable topoisomerase inhibitors include, but are not limited to, amsacrine, etoposide (VP-16), irinotecan, mitoxantrone, RFS 2000, teniposide, and topotecan.

Non-limiting examples of suitable anti-hormonal agents such as aminoglutethimide, antiestrogen, aromatase inhibiting 4 (5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, and trilostane.

Examples of targeted therapeutic agents include, without limit, monoclonal antibodies such as alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, and vandetanib; angiogeneisis inhibitors such as angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, and thalidomide; and growth inhibitory polypeptides such as bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1BB ligand, and Apo-1 ligand.

Non-limiting examples of photodynamic therapeutic agents include aminolevulinic acid, methyl aminolevulinate, retinoids (alitretinon, tamibarotene, tretinoin), and temoporfin.

Other antineoplastic agents include anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, and vorinostat.

In certain embodiments, the additional pharmaceutical agent is another Bcl-2 inhibitor. In certain embodiments, the another Bcl-2 inhibitor is ABT-737, navitoclax (ABT-263), venetoclax (ABT-199), obatoclax (GX 15-070), (−)-gossypol (AT-101), sabutoclax (BI-97C₁), TW-37, BM-1252 (APG-1252), or A-1155463. In certain embodiments, the other Bcl-2 inhibitor is venetoclax or ABT-263. In certain embodiments, the additional pharmaceutical agent is another Bcl-xL inhibitor. In certain embodiments, the another Bcl-xL inhibitor is ABT-263, ABT-737, A-1155463, A-1331852, AZD4320, WEHI-539, (−)-gossypol, BH3I-1, berberine, flavokawain A, sabutoclax, TW-37, or gambogic acid.

In certain embodiments, the additional pharmaceutical agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-1 antibody, an anti-PD-L₁ antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, MGA271, IMC-CS4, emactuzumab, pertuzumab, obinutuzumab, cabiralizumab, margetuximab, enoblituzumab, mogamulizumab, carlumab, bevacizumab, trastuzumab (HERCEPTIN®), bevacizumab (AVASTIN®), rituximab (RITUXAN®), cetuximab (ERBITUX®), panitumumab (VECTIBIX®), alemtuzumab (CAMPATH®), or ranibizumab (Lucentis®).

In certain embodiments, at least one of the additional pharmaceutical agents is a cytotoxic agent. In certain embodiments, at least one of the additional pharmaceutical agents is venetoclax, azacitidine, bortezomib, cladribine, cytarabine, doxorubicin, eribulin, etoposide, everolimus, ixabepilone, mitoxantrone, ixazomib, panobinostat, pemetrexed, SN-38, or topotecan. In certain embodiments, at least one of the additional pharmaceutical agents is nivolumab, pidilizumab, pembrolizumab, MEDI-0680, REGN2810, AMP-224, atezolizumab, durvalumab, BMS-936559, avelumab, CA-170, ipilimumab, or tremelimumab.

In certain embodiments, at least one of the additional pharmaceutical agents is an immune check-point inhibitor. In certain embodiments, at least one of the additional pharmaceutical agents is a cytotoxic T-lymphocyte-associated protein 4 (CTLA-4) inhibitor. In certain embodiments, at least one of the additional pharmaceutical agents is a programmed cell death 1 protein (PD-1) inhibitor. In certain embodiments, at least one of the additional pharmaceutical agents is a programmed cell death 1 protein ligand 1 (PD-L₁) inhibitor. In certain embodiments, at least one of the additional pharmaceutical agents is a stimulator of interferon genes (STING) agonist. In certain embodiments, the additional pharmaceutical agents include pharmaceutically acceptable salts, solvates, hydrates, polymorphs, co-crystals, tautomers, stereoisomers, isotopically labeled compounds, and prodrug thereof.

In certain embodiments, the provided compounds or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) agent.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In another aspect, the present disclosure provide a kit comprising a provided compound or pharmaceutical composition, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of treatment or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of

Medicinal Products (EMA). In certain embodiments, the instructions comprise prescribing information.

In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. In certain embodiments, the kits and instructions provide for inhibiting the activity (e.g., aberrant activity, such as increased activity) of Bcl-xL and/or Bcl-2 in a subject or cell. A kit described herein may include one or more additional pharmaceutical agents described herein as a separate pharmaceutical composition.

### Methods of Treatment

In another aspect, the present disclosure provides a method of degrading a Bcl-2 protein in a cell, tissue, or biological sample, the method comprising contacting the cell, tissue, or biological sample with an effective amount of a compound or pharmaceutical composition provided herein.

In another aspect, the present disclosure provides a method of degrading a Bcl-xL protein in a cell, tissue, or biological sample, the method comprising contacting the cell, tissue, or biological sample with an effective amount of a compound or pharmaceutical composition provided herein.

In another aspect, the present disclosure provides a method of treating a disease in a subject in need thereof, the method comprising administering to the subject an effective amount of a compound or pharmaceutical composition provided herein.

In another aspect, the present disclosure provides a method of preventing a disease in a subject in need thereof, the method comprising administering to the subject an effective amount of a compound or pharmaceutical composition provided herein.

In another aspect, the present disclosure provides a method of selectively killing one or more cancer cells in a tissue or biological sample, the method comprising contacting the tissue or biological sample with an effective amount of a compound or pharmaceutical composition of the present disclosure

In another aspect, the present disclosure provides a method of selectively killing one or more cancer cells in a subject in need thereof, the method comprising administering to the subject an effective amount of a compound or pharmaceutical composition of the present disclosure.

By “selectively killing one or more cancer cells,” it is meant that a composition provided herein does not appreciably kill non-cancer cells at the same concentration. In one embodiment, a composition provided herein has reduced platelet toxicity and retained or improved toxicity in cancer cells when compared to other Bcl-2 and/or Bcl-xL inhibitors. Accordingly, the median lethal dose or LD₅₀ of the inhibitor in non-cancer cells may be about 5 to about 50 times higher than the LD₅₀ of the inhibitor in cancer cells. As used herein, the LD₅₀ is the concentration of inhibitor required to kill half the cells in the cell sample. For example, the LD₅₀ of the inhibitor in non-cancer cells may be greater than about 5, about 6, about 7, about 8, about 9 or about 10 times higher than the LD₅₀ of the inhibitor in cancer cells. Alternatively, the LD₅₀ of the inhibitor in non-cancer cells may be greater than about 10, about 15, about 20, about 25, about 30, about 35, about 40, about 45, or about 50 times higher than the LD₅₀ of the inhibitor in cancer cells.

Additionally, the LD₅₀ of the inhibitor in non-cancer cells may be greater than 50 times higher than the LD₅₀ of the inhibitor in cancer cells. In a specific embodiment, the LD₅₀ of the inhibitor in non-cancer cells is greater than 10 times higher than the LD₅₀₀ of the inhibitor in cancer cells. In another specific embodiment, the LD₅₀ of the inhibitor in non-cancer cells is greater than 20 times higher than the LD₅₀ of the inhibitor in cancer cells.

In certain embodiments, the cell, tissue, biological sample, or cancer cell is in vitro. In certain embodiments, the cell, tissue, biological sample, or cancer cell is in vivo. In certain embodiments, the cell, tissue, or biological sample is ex vivo. In certain embodiments, the cell is a cancer cell. In certain embodiments, the cell is a pre-cancer cell. In certain embodiments, the cell, tissue, or biological sample is in a subject.

In certain embodiments, the disease is cancer. In certain embodiments, the cancer is a Bcl-2-mediated cancer. In certain embodiments, the cancer is a Bcl-xL-mediated cancer. In certain embodiments, the cancer is a hematological malignancy. In certain embodiments, the cancer is leukemia. In certain embodiments, the cancer is acute myeloid leukemia, chronic lymphocytic leukemia, or acute lymphoblastic leukemia. In certain embodiments, the cancer is lymphoma. In certain embodiments, the cancer is T-cell lymphoma. In certain embodiments, the cancer is a solid tumor. In certain embodiments, the anticancer activity is measured in MOLT-4 cells. In certain embodiments, the anticancer activity is measured in RS4 cells. In some embodiments, the anticancer activity is higher in MOLT-4 cells than in RS4 cells. In certain embodiments, the anticancer activity is higher in RS4 cells than in MOLT-4 cells. In certain embodiments, the ratio of the anticancer activity in MOLT-4 cells to the anticancer activity in RS4 cells is between 2:1 and 5:1, between 5:1 and 10:1, between 10:1 and 30:1, between 30:1 and 100:1, between 100:1 and 300:1, or between 300:1 and 1000:1, or greater than 1000:1. In certain embodiments, the ratio of the anticancer activity in RS4 cells to the anticancer activity in MOLT-4 cells is between 2:1 and 5:1, between 5:1 and 10:1, between 10:1 and 30:1, between 30:1 and 100:1, between 100:1 and 300:1, or between 300:1 and 1000:1, or greater than 1000:1.

It has been reported that senolytics have the potential to prevent and treat a growing number of age-related diseases and extend healthspan, and that senolytics include inhibitors of the antiapoptotic Bcl-2 family proteins (He et al., Nat. Commun., 11, 1996 (2020); Kirkland et al., J. Intern. Med., 2020, 288:518-536). In certain embodiments, the disease is an age-related disease. In certain embodiments, the disease is age-related cognitive dysfunction, age-related intervertebral disc disease, age-related muscle loss, Alzheimer's disease, amyotrophic lateral sclerosis, ataxia, AV fistulae, bone marrow transplant complication, cardiac dysfunction, cataracts, chemotherapy complication, chronic obstructive, cirrhosis, COVID-19 complication, diabetes, frailty, glaucoma, hepatic steatosis, hyperoxic lung damage, idiopathic pulmonary fibrosis, macular degeneration, MGUS, myeloma, obesity, obesity-related neuropsychiatric dysfunction, organ transplantation complication, osteoarthritis, osteoporosis, Parkinson's disease, pre-eclampsia, primary biliary cirrhosis, progerias, prostatic hypertrophy, psoriasis, pulmonary disease, radiation complication, renal dysfunction, urinary incontinence, vascular calcification, or vascular hyporeactivity.

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years. In certain embodiments, the subject is a human younger than 2 years. In certain embodiments, the subject is a non-human animal. In certain embodiments, the subject is a mammal. In certain embodiments, the subject is a non-human mammal.

### EXAMPLES

The present disclosure will now be demonstrated using specific examples that are not to be construed as limiting.

### Preparation of Intermediate A

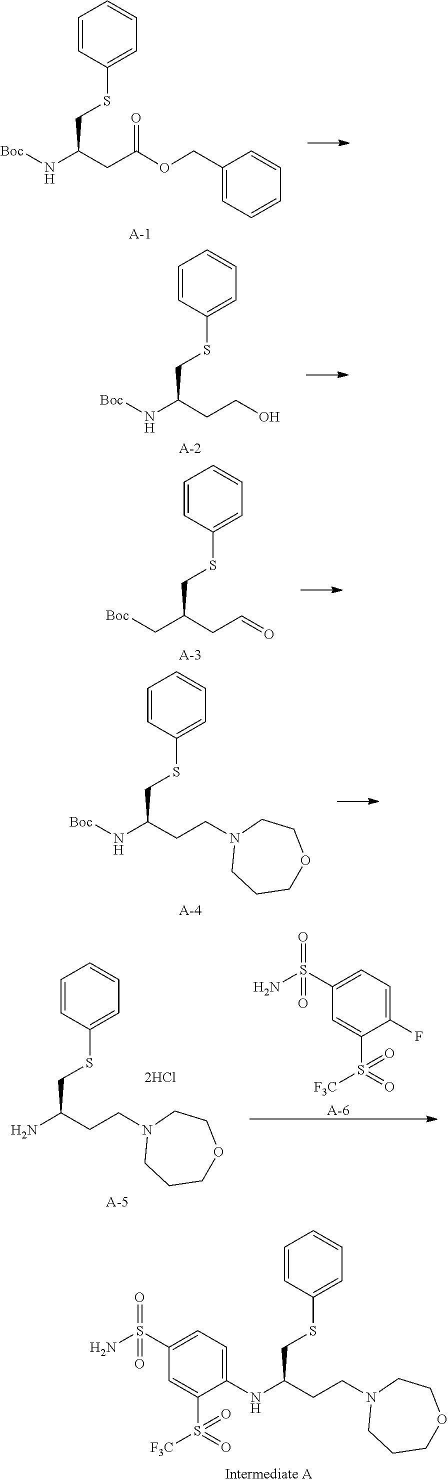

Step 1: Synthesis of tert-butyl (R)-(4-hydroxy-1-(phenylthio) butan-2-yl) carbamate (A-2). To a solution of A-1 (2.7 g, 6.72 mmol) in THF/MeOH (50 mL/5 mL) was added NaBH₄ (1.49 g, 40.35 mmol) in portions over a period of 6 hours. The reaction mixture was added sat. aq. NH₄Cl and extracted with DCM. The combined organic layers were washed with sat. aq. NH₄Cl, then dried over Na₂SO₄, filtered and concentrated. The residue was purified by flash column chromatography (0% to 70% of EtOAc in hexanes) to afford A-2 (1.6 g, 80% yield) as a white solid. ¹H NMR (600 MHZ, Chloroform-d) δ (ppm): 7.43-7.40 (m, 2H), 7.32 (t, J=7.8 Hz, 2H), 7.26-7.21 (m, 1H), 4.84 (d, J=8.4 Hz, 1H), 4.07 (s, 1H), 3.67 (s, 2H), 3.22-2.98 (m, 3H), 1.98-1.82 (m, 1H), 1.67-1.49 (m, 1H), 1.46 (s, 9H). LC/MS (ESI) m/z 320.2 [M+Na]⁺; [M+H]⁺ calcd for C₁₅H₂₄NO₃S⁺: 298.15.

Step 2: Synthesis of tert-butyl (R)-(4-oxo-1-(phenylthio) butan-2-yl) carbamate (A-3). DMP (2.08 g, 4.90 mmol) was added to a solution of A-2 (1.08 g, 3.63 mmol) in EtOAc (25 mL) and stirred at room temperature for 3 hours. The resulting mixture was filtered and the filtrate was concentrated. The residue was purified by flash column chromatography (0% to 45% of EtOAc in hexanes) to afford A-3 (1.0 g, 93% yield) as a colorless oil. ¹H NMR (600 MHZ, Chloroform-d) δ 9.71 (s, 1H), 7.43-7.40 (m, 2H), 7.35-7.30 (m, 2H), 7.26-7.21 (m, 1H), 5.00 (s, 1H), 4.22 (s, 1H), 3.33-3.21 (m, 1H), 3.17-3.07 (m, 1H), 2.90-2.72 (m, 2H), 1.44 (s, 9H). LC/MS (ESI) m/z 296.2; [M+H]⁺ calcd for C₁₅H₂₂NO₃S⁺: 296.13.

Step 3: Synthesis of tert-butyl (R)-(4-(1,4-oxazepan-4-yl)-1-(phenylthio) butan-2-yl) carbamate (A-4). NaBH(OAc)₃ (632 mg, 2.98 mmol) was added to a solution of 1,4-oxazepane hydrochloride (410 mg, 2.98 mmol), TEA (565 μL, 4.06 mmol) and A-3 (800 mg, 2.71 mmol) in DCM (15 mL), then stirred at room temperature overnight. The reaction mixture was washed with sat. aq. NH₄Cl. The organic layers were dried over Na₂SO₄, filtered and concentrated. The residue was purified by flash column chromatography (50% to 100% of EtOAc in hexanes, then 0% to 10% of MeOH in DCM) to afford A-4 (920 mg, 89% yield) as a white solid. ¹H NMR (600 MHZ, Chloroform-d) δ 7.42 (d, J=7.6 Hz, 2H), 7.33-7.29 (m, 2H), 7.19 (t, J=7.4 Hz, 1H), 3.91 (s, 1H), 3.82 (t, J=6.1 Hz, 2H), 3.73 (t, 2H), 3.30 (d, J=13.5 Hz, 1H), 3.07-2.95 (m, 1H), 2.77-2.60 (m, 4H), 2.59-2.52 (m, 1H), 1.95-1.82 (m, 3H), 1.74-1.66 (m, 1H), 1.45 (s, 9H). LC/MS (ESI) m/z 381.4; [M+H]⁺ calcd for C₂₀H₃₃N₂O₃S⁺: 381.22.

Step 4: Synthesis of (R)-4-(1,4-oxazepan-4-yl)-1-(phenylthio) butan-2-amine hydrochloride (A-5). 4 N HCl in dioxane (10 mL) was added to a stirring solution of A-4 (730 mg, 1.92 mmol) in DCM (10 mL). After stirring at room temperature for 1 hour, the reaction mixture was concentrated to afford A-5 (850 mg, quantitative yield), which was directly used in the next step. LC/MS (ESI) m/z 281.1; [M+H]⁺ calcd for C₁₅H₂₅N₂OS⁺: 281.17.

Step 5: Synthesis of (R)-4-((4-(1,4-oxazepan-4-yl)-1-(phenylthio) butan-2-yl)amino)-3-((trifluoromethyl) sulfonyl)benzenesulfonamide (intermediate A). A-6 (600 mg, 1.95 mmol) was added to a stirring solution of A-5 (850 mg, HCl salt) and DIPEA (2.72 mL, 15.64 mmol) in DMSO (25 mL), then stirred at room temperature overnight. The resulting mixture was diluted with EtOAc and washed with sat. aq. NH₄Cl. The organic layer was dried over Na₂SO₄, filtered, and concentrated. The residue was purified by flash column chromatography (50% to 100% of 20 EtOAc in hexanes, then 0% to 10% of MeOH in DCM) to afford Intermediate A (900 mg, 82% yield for two steps) as a yellow solid. ¹H NMR (600 MHZ, Chloroform-d) δ 8.27 (d, J=2.2 Hz, 1H), 7.83 (dd, J=9.2, 2.3 Hz, 1H), 7.44-7.40 (m, 2H), 7.37-7.33 (m, 2H), 7.33-7.27 (m, 1H), 7.19 (d, J=8.5 Hz, 1H), 6.65 (d, J=9.2 Hz, 1H), 5.06 (s, 2H), 4.01-3.91 (m, 1H), 3.77 (t, J=6.0 Hz, 2H), 3.73-3.63 (m, 2H), 3.17-3.03 (m, 2H), 2.75-2.69 (m, 2H), 2.68-2.52 (m, 4H), 2.14-2.04 (m, 1H), 1.90-1.82 (m, 2H), 1.76-1.67 (m, 1H). LC/MS (ESI) m/z 568.1; [M+H]⁺ calcd for C₂₂H₂₉F₃N₃O₅S₃⁺: 568.12.

### Preparation of Intermediate B

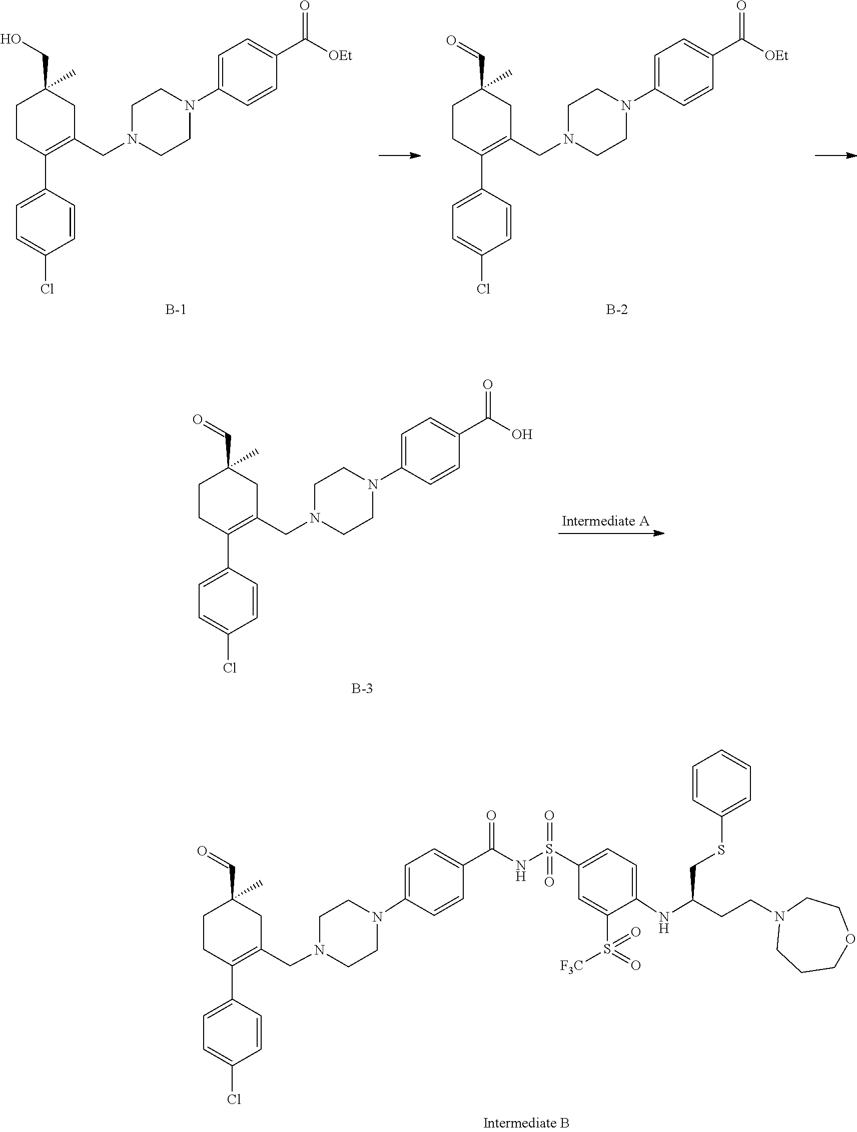

Step 1: Synthesis of ethyl (R)-4-(4-((4′-chloro-4-formyl-4-methyl-3,4,5,6-tetrahydro-[1,1′-biphenyl]-2-yl)methyl) piperazin-1-yl)benzoate (B-2). Under an argon atmosphere, to a solution of DMSO (1.76 mL, 24.84 mmol) in DCM (30 mL) was added oxalyl chloride (1.3 mL, 15.53 mmol) dropwise at −78° C. with stirring. The resulting mixture was stirred at −78° C. for an additional 15 min. Then a solution of B-1 (4 g, 6.21 mmol) in DCM/DMSO (20 mL/5 mL) was added dropwise. The mixture was stirred at −78° C. for an extra 30 min after completing the addition. TEA (6.9 mL, 49.69 mmol) was added to the mixture at −78° C., then dry-ice/acetone bath was removed. The reaction mixture was warmed up to room temperature and stirred for an additional 30 min. Water was added to the reaction mixture, then extracted with DCM. The combined organic layers were washed with 10% aq. Na₂S₂O₃ and sat. aq. NH₄Cl, dried over Na₂SO₄, filtered, and concentrated. The residue was purified by flash column chromatography (0% to 40% of EtOAc in hexanes) to afford B-2 (2.82 g, 71%) as an off-white solid. ¹H NMR (600 MHZ, Chloroform-d) δ (ppm): 9.56 (s, 1H), 7.95-7.89 (m, 2H), 7.33-7.27 (m, 2H), 7.01-6.95 (m, 2H), 6.87-6.81 (m, 2H), 4.34 (q, J=7.1 Hz, 2H), 3.30 (t, J=5.1 Hz, 4H), 2.86 (q, J=12.7 Hz, 2H), 2.73-2.66 (m, 1H), 2.48-2.41 (m, 2H), 2.40-2.26 (m, 4H), 2.12-2.04 (m, 1H), 2.05-1.99 (m, 1H), 1.69-1.60 (m, 1H), 1.38 (t, J=7.1 Hz, 3H), 1.18 (s, 3H). LC/MS (ESI) m/z 481.2; [M+H]⁺ calcd for C₂₈H₃₄ClN₂O₃⁺: 481.23.

Step 2: Synthesis of (R)-4-(4-((4′-chloro-4-formyl-4-methyl-3,4,5,6-tetrahydro-[1,1′-biphenyl]-2-yl)methyl) piperazin-1-yl)benzoic acid (B-3). To a solution of B-2 (2.3 g, 4.78 mmol) in MeOH/THF (25 mL/25 mL) was added a solution of LiOH·H₂O (602 mg, 14.35 mmol) in water (6 mL), then stirred at 40° C. overnight. The reaction mixture was concentrated to remove the organic solvents, then adjusted to pH=6 with 10% aq. citric acid. The precipitate was collected via filtration and washed with water. The filtered cake was dried under vacuum to afford B-3 (2.0 g, 92% yield) as a white solid. ¹H NMR (600 MHz, Methanol-d₄) δ 9.56 (s, 1H), 7.91 (d, J=9.0 Hz, 2H), 7.39 (d, J=8.4 Hz, 2H), 7.10 (d, J=8.5 Hz, 2H), 6.98 (d, J=9.0 Hz, 2H), 3.52-3.42 (m, 5H), 2.97 (s, 4H), 2.68 (dd, J=17.5, 1.9 Hz, 1H), 2.44-2.33 (m, 3H), 2.14-2.06 (m, 2H), 1.77-1.69 (m, 1H), 1.21 (s, 3H). LC/MS (ESI) m/z 453.2; [M+H]⁺ calcd for C₂₆H₃₀ClN₂O₃⁺: 453.19.

Step 3: Synthesis of N-((4-(((R)-4-(1,4-Oxazepan-4-yl)-1-(phenylthio) butan-2-yl)amino)-3-((trifluoromethyl) sulfonyl)phenyl) sulfonyl)-4-(4-(((R)-4′-chloro-4-formyl-4-methyl-3,4,5,6-tetrahydro-[1,1′-biphenyl]-2-yl)methyl) piperazin-1-yl)benzamide (intermediate B). Intermediate A (853 mg, 1.5 mmol) was added to a solution of B-3 (680 mg, 1.5 mmol), DMAP (916 mg, 7.51 mmol) and EDC (1.49 g, 7.51 mmol) in DCM (15 mL). The resulting mixture was stirred at room temperature overnight. The reaction mixture was washed with water and sat. aq. NH₄Cl, dried over Na₂SO₄, filtered, and concentrated. The residue was purified by flash column chromatography (0% to 10% of MeOH in EtOAc) to afford Intermediate B (840 mg, 56% yield). ¹H NMR (600 MHZ, DMSO-d₆) δ 9.52 (s, 1H), 8.09 (d, J=2.2 Hz, 1H), 7.94 (dd, J=9.0, 2.2 Hz, 1H), 7.71 (d, J=8.6 Hz, 2H), 7.37 (d, J=8.2 Hz, 2H), 7.34 (d, J=7.7 Hz, 2H), 7.29 (t, J =7.6 Hz, 2H), 7.22-7.17 (m, 1H), 7.08 (d, J=8.2 Hz, 2H), 6.89 (d, J=9.3 Hz, 1H), 6.79 (dd, J=18.3, 8.6 Hz, 3H), 4.05-3.96 (m, 1H), 3.63 (t, J=6.1 Hz, 4H), 3.39-3.29 (m, 7H), 3.26 (dd, J=13.9, 6.7 Hz, 1H), 3.20-3.11 (m, 4H), 2.85-2.71 (m, 3H), 2.55 (d, J=17.6 Hz, 1H), 2.35-2.17 (m, 6H), 2.06-1.95 (m, 2H), 1.92-1.74 (m, 4H), 1.57 (dt, J=13.5, 6.9 Hz, 1H), 1.08 (s, 3H). LC/MS (ESI) m/z 1002.4; [M+H]⁺ calcd for C₄₈H₅₆ClF₃N₅O₇S₃⁺: 1002.3.

### Preparation of Intermediate C

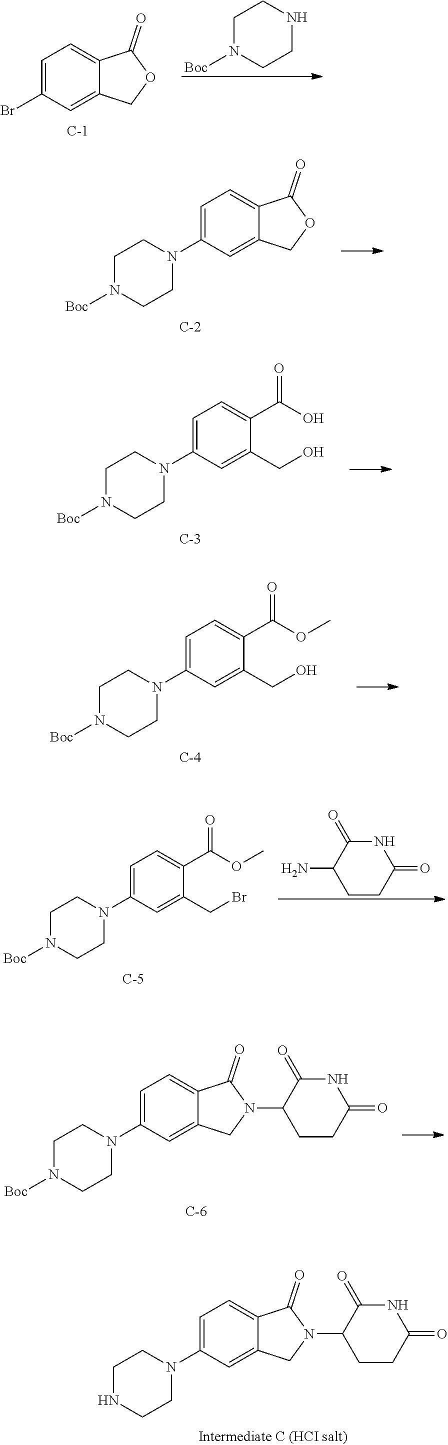

Step 1: Synthesis of tert-butyl 4-(1-ox0-1,3-dihydroisobenzofuran-5-yl) piperazine-1-carboxylate (C-2). Under an argon atmosphere, a mixture of C-1 (4 g, 18.78 mmol), 4-Boc-piperazine (5.24 g, 28.17 mmol), Pd2 (dba)₃ (860 mg, 0.94 mmol), BINAP (1.17 g, 1.88 mmol), and K₃PO₄ (11.9 g, 56.34 mmol) in toluene (100 mL) was stirred at 90° C. overnight. After cooling to room temperature, the reaction mixture was washed with water and sat. aq. NH₄Cl. The organic layers were dried over Na₂SO₄, filtered and concentrated. The residue was purified by flash column chromatography (0% to 10% of MeOH in DCM) to afford C-2 (2.2 g, 37% yield). ¹H NMR (600 MHZ, Chloroform-d) δ 7.78 (d, J=8.6 Hz, 1H), 7.01 (dd, J=8.6, 2.2 Hz, 1H), 6.82 (d, J=2.2 Hz, 1H), 5.23 (s, 2H), 3.63 (t, 4H), 3.38 (t, J=5.3 Hz, 4H), 1.51 (s, 8H). LC/MS (ESI) m/z 319.1; [M+H]⁺ calcd for C₁₇H₂₃N₂O₄⁺: 319.17.

Step 2: Synthesis of 4-(4-(tert-butoxycarbonyl) piperazin-1-yl)-2-(hydroxymethyl)benzoic acid (C-3). To a solution of B-2 (2.2 g, 6.92 mmol) in MeOH/THF (10 mL/10 mL) was added a solution of LiOH.H₂O (553 mg, 13.84 mmol) in water (5 mL). The resulting mixture was stirred at room temperature for 2.5 hours. The reaction mixture was concentrated to remove the organic solvents, then extracted with EtOAc. The water phase was adjusted to pH=3-4 with 5% aq. citric acid. The precipitate was collected via filtration and washed with water. The filtered cake was dried under vacuum to afford C-3 (2.2 g, 96% yield) as a light-yellow solid. ¹H NMR (600 MHz, Chloroform-d) δ 8.07 (d, J=8.8 Hz, 1H), 6.91 (d, J=2.7 Hz, 1H), 6.81 (dd, J=8.9, 2.7 Hz, 1H), 4.81 (s, 2H), 3.61 (t, J=5.3 Hz, 4H), 3.40 (t, J=5.3 Hz, 4H), 1.51 (s, 9H). LC/MS (ESI) m/z 337.2; [M+H]⁺ calcd for C₁₇H₂₅N₂O₅⁺: 337.18.

Step 3: Synthesis of tert-butyl 4-(3-(hydroxymethyl)-4-(methoxycarbonyl)phenyl) piperazine-1-carboxylate (C-4). (Trimethylsilyl)diazomethane (10% in hexanes, 22.4 g, 19.64 mmol) was added dropwise to a stirring solution of C-3 (2.2 g, 6.55 mmol) in MeOH/EtOAc (50 mL/50 mL) at room temperature. After completing the addition, the resulting mixture was stirred at room temperature for an extra 30 min. The reaction mixture was concentrated and the residue was purified by flash column chromatography (0% to 60% of EtOAc in hexanes) to afford C-4 (2.2 g, 96% yield). ¹H NMR (600 MHZ, Chloroform-d) δ 7.97 (dd, J=8.8, 1.3 Hz, 1H), 6.88 (d, J=2.7 Hz, 1H), 6.81-6.77 (m, 1H), 4.75 (d, J=7.5 Hz, 2H), 3.91 (s, 3H), 3.60 (t, J=5.2 Hz, 4H), 3.36 (t, J=5.4 Hz, 4H), 1.51 (s, 9H). LC/MS (ESI) m/z 351.2; [M+H]⁺ calcd for C₁₈H₂₇N₂O₅⁺: 351.19.

Step 4: Synthesis of tert-butyl 4-(3-(bromomethyl)-4-(methoxycarbonyl)phenyl) piperazine-1-carboxylate (C-5). Under argon atmosphere, to a solution of C-4 (2.2 g, 6.29 mmol) in THF (60 mL) were added triphenylphosphine (2.5 g, 9.43 mmol) and carbon tetrabromide (3.1 g, 9.43 mmol). The resulting mixture was stirred at room temperature for 1 hour, then washed with water and brine. The organic layers were dried over Na₂SO₄, filtered, and concentrated. The residue was purified by flash column chromatography (0% to 50% of EtOAc in hexanes) to afford C-5 (2.24 g, 86% yield) as an off-white solid. ¹H NMR (600 MHz, Chloroform-d) δ 7.96 (d, J=8.8 Hz, 1H), 6.91 (d, J=2.7 Hz, 1H), 6.80 (dd, J=8.9, 2.7 Hz, 1H), 4.99 (s, 2H), 3.91 (s, 3H), 3.61 (t, 4H), 3.34 (t, J=5.3 Hz, 4H), 1.51 (s, 9H). LC/MS (ESI) m/z 313.1, 315.1 (M-Boc); [M+H]⁺ calcd for C₁₈H₂₅BrN₂O₄⁺: 413.11.

Step 5: Synthesis of tert-butyl 4-(2-(2,6-dioxopiperidin-3-yl)-1-oxoisoindolin-5-yl) piperazine-1-carboxylate (C-6). A solution of C-5 (2.2 g, 5.33 mmol), 3-aminopiperidine-2,6-dione hydrochloride (1.31 g, 8.0 mmol), and DIPEA (4.6 mL, 26.63 mmol) in acetonitrile (30 mL) was stirred at 70° C. overnight. After cooling to room temperature, the reaction mixture was concentrated. Water was added to the residue, then collected the precipitate via filtration. The filtered cake was washed with water, DCM and DCM/Et20 (v/v=1/1), then dried under vacuum to afford C-6 (1.43 g, 63% yield). ¹H NMR (600 MHZ, Chloroform-d) δ 8.07 (s, 1H), 7.77 (d, J=8.5 Hz, 1H), 7.01 (dd, J=8.6, 2.2 Hz, 1H), 6.90 (d, J=2.2 Hz, 1H), 5.22 (dd, J=13.3, 5.1 Hz, 1H), 4.44 (d, J=15.6 Hz, 1H), 4.28 (d, J=15.6 Hz, 1H), 3.62 (t, J=5.3 Hz, 4H), 3.30 (t, J=5.2 Hz, 4H), 2.98-2.77 (m, 2H), 2.35 (qd, J=13.2, 4.8 Hz, 1H), 2.26-2.17 (m, 1H), 1.51 (s, 9H). LC/MS (ESI) m/z 429.2; [M+H]⁺ calcd for C₂₂H₂₉N₂O₅⁺: 429.21.

Step 6: Synthesis of 3-(1-oxo-5-(piperazin-1-yl) isoindolin-2-yl) piperidine-2,6-dione hydrochloride (Intermediate C). HCl (10 mL, 4 N in dioxane) was added to a stirring solution of C-6 (1 g, 2.34 mmol) in DCM (10 mL) at room temperature. The resulting mixture was stirred at room temperature for 2 hours, the reaction mixture was concentrated and the residue was triturated with diethyl ether. The precipitate was collected via filtration and dried under vacuum to afford Intermediate C (1.04 g, quantitative yield), which was directly used in the next step. LC/MS (ESI) m/z 329.2; [M+H]⁺ calcd for C₁₇H₂₁N₄O₃⁺: 329.16.

### Preparation of Intermediate D

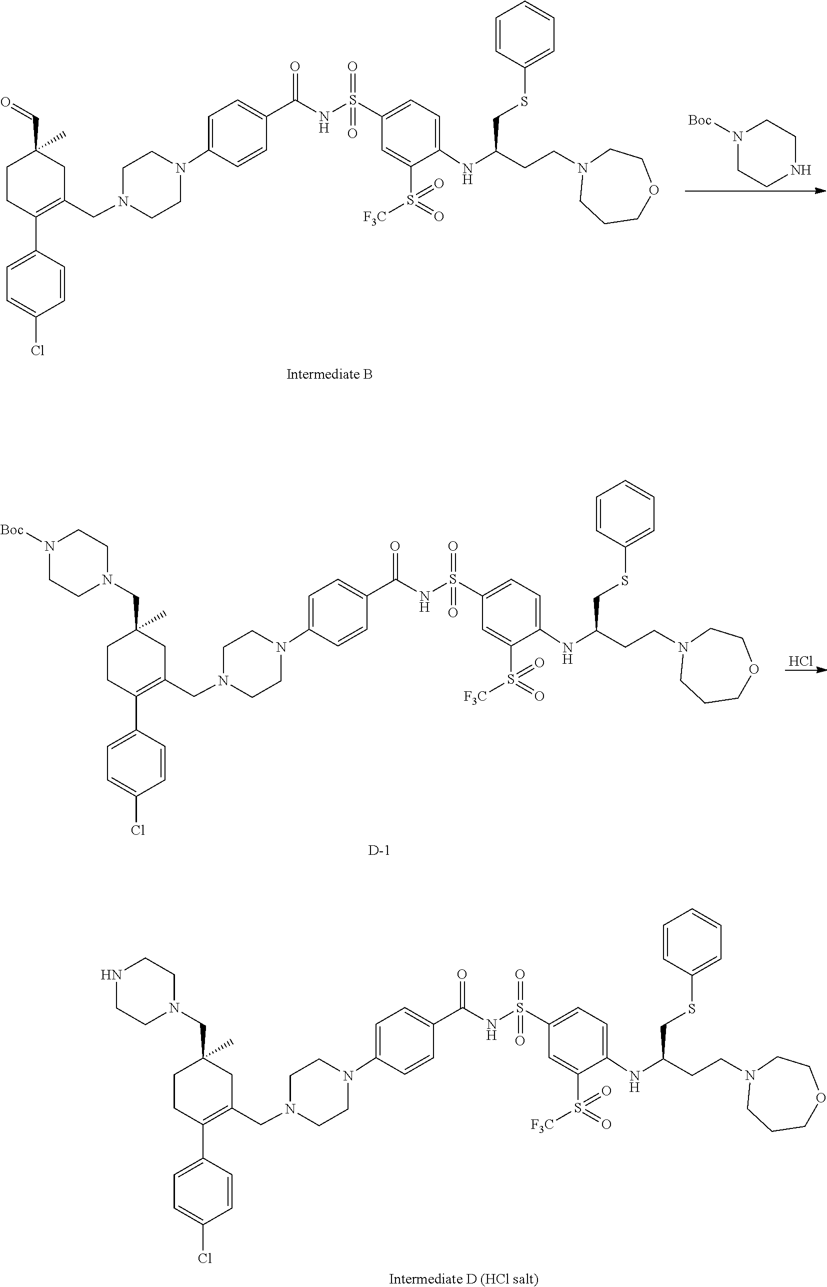

Intermediates D was synthesized according to the procedure described in WO 2023107606.

### Preparation of Intermediate E

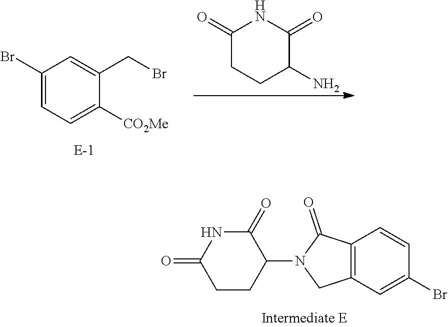 [SMILES: COC(=O)c1ccc(Br)cc1CBr ; NC1CCC(=O)NC1=O ; O=C1CCC(N2Cc3cc(Br)ccc3C2=O)C(=O)N1]

Synthesis of 3-(5-bromo-1-oxoisoindolin-2-yl) piperidine-2,6-dione (Intermediate E). A solution of E-1 (6.7 g, 21.89 mmol), 3-aminopiperidine-2,6-dione hydrochloride (3.6 g, 21.89 mmol), and DIPEA (11.4 mL, 65.69 mmol) in acetonitrile (120 mL) was stirred at 70° C. overnight. After cooling to room temperature, the reaction mixture was concentrated. The residue was triturated with water and DCM, then the precipitate was collected via filtration and dried under vacuum to afford Intermediate E (5.1 g, 72% yield). ¹H NMR (600 MHZ, DMSO-d₆) δ 11.01 (s, 1H), 7.90 (d, J=1.6 Hz, 1H), 7.72 (dd, J=8.1, 1.7 Hz, 1H), 7.68 (d, J=8.0 Hz, 1H), 5.12 (dd, J=13.3, 5.2 Hz, 1H), 4.47 (d, J=17.6 Hz, 1H), 4.35 (d, J=17.5 Hz, 1H), 2.91 (ddd, J=17.4, 13.6, 5.4 Hz, 1H), 2.61 (ddd, J=17.4, 4.5, 2.2 Hz, 1H), 2.40 (qd, J=13.3, 4.5 Hz, 1H), 2.05-1.97 (m, 1H). LC/MS (ESI) m/z 323.1, 325.1; [M+H]⁺ calcd for C₁₃H₁₂BrN₂O₃⁺: 323.00.

### Preparation of Intermediates F and G

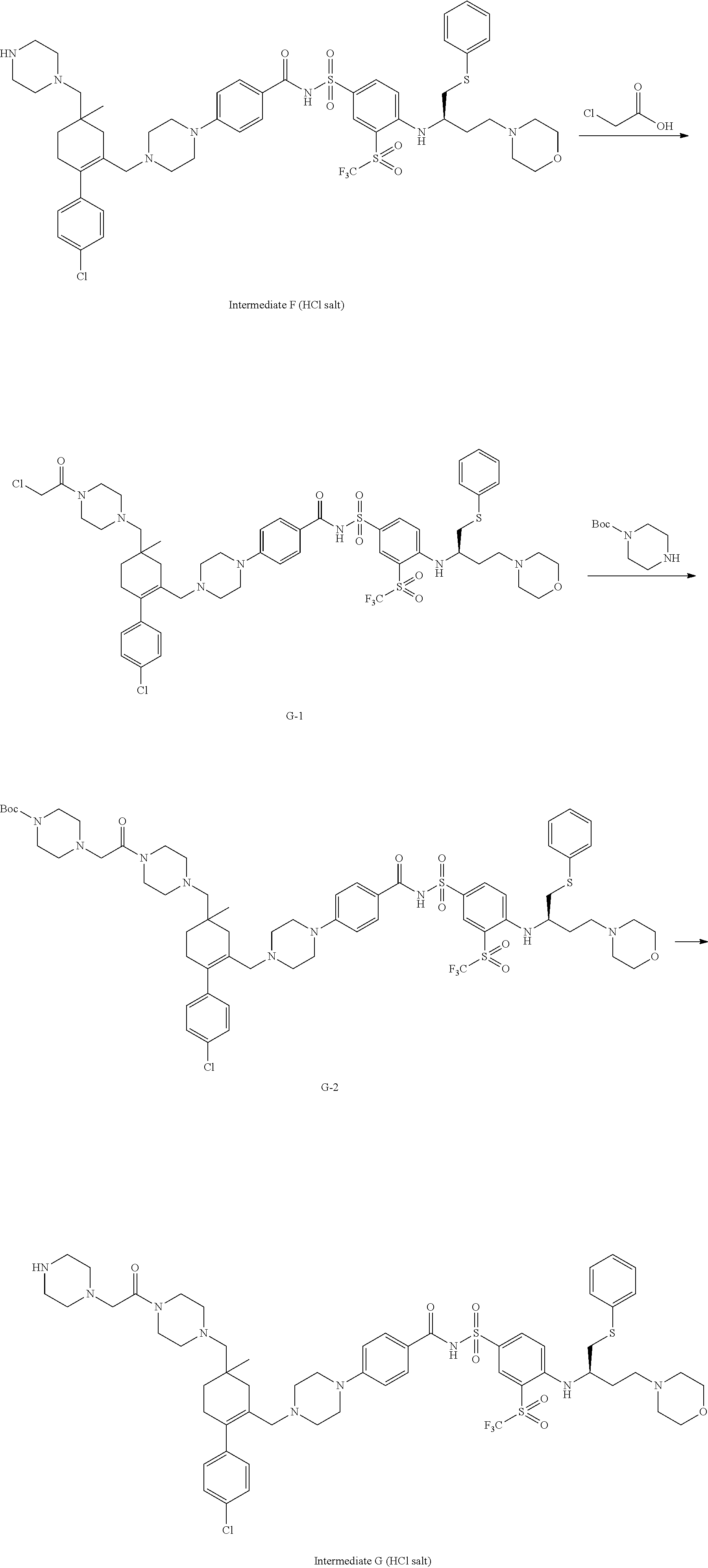

Step 1: Synthesis of 4-(4-((4′-chloro-4-((4-(2-chloroacetyl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-((trifluoromethyl) sulfonyl)phenyl) sulfonyl)benzamide (G-1). To a solution of 2-chloroacetic acid (8.6 mg, 0.091 mmol) and HATU (43 mg, 0.11 mmol) in (1 mL) was added a solution of Intermediate F (100 mg, 0.091 mmol. Intermediate F was synthesized according to WO 2023107606), and DIPEA (25 μL, 0.14 mmol) in DCM (0.5 mL). The resulting mixture was stirred at room temperature for 2 hours, then washed with water and sat. aq. NH₄Cl. The organic layers were dried over Na₂SO₄, filtered and concentrated. The residue was purified by flash column chromatography (0% to 5% of DCM in MeOH) to afford G-1 (75 mg, 72% yield). ¹H NMR (600 MHZ, Chloroform-d) δ 8.35 (d, J=2.3 Hz, 1H), 8.09 (d, J=9.1 Hz, 1H), 7.66 (d, J=8.5 Hz, 2H), 7.40-7.34 (m, 2H), 7.34-7.22 (m, 6H), 7.04 (d, J=8.6 Hz, 1H), 6.99 (d, J=8.0 Hz, 2H), 6.75 (s, 2H), 6.61 (d, J=9.3 Hz, 1H), 4.04 (s, 2H), 3.90 (d, J=9.9 Hz, 1H), 3.72-3.55 (m, 6H), 3.50 (s, 2H), 3.27 (s, 4H), 3.10 (dd, J=13.9, 5.1 Hz, 1H), 3.02 (dd, J=13.9, 7.2 Hz, 1H), 2.96-2.82 (m, 2H), 2.69-2.49 (m, 4H), 2.49-2.18 (m, 14H), 2.17-2.07 (m, 2H), 2.02-1.93 (m, 1H), 1.73-1.52 (m, 2H), 1.51-1.42 (m, 1H), 0.96 (s, 3H). LC/MS (ESI) m/z 1134.4; [M+H]⁺ calcd for C₅₃H₆₅C₁₂F₃N₇O₇S₃⁺: 1134.34.

Step 2: Synthesis of tert-butyl 4-(2-(4-((4′-chloro-4-methyl-6-((4-(4-(((4-(((R)-4-morpholino-1-(phenylthio) butan-2-yl)amino)-3-((trifluoromethyl) sulfonyl)phenyl) sulfonyl) carbamoyl)phenyl) piperazin-1-yl)methyl)-2,3,4,5-tetrahydro-[1,1′-biphenyl]-4-yl)methyl) piperazin-1-yl)-2-oxoethyl) piperazine-1-carboxylate (G-2). A mixture of G-1 (72 mg, 0.063 mmol), N-Boc-piperazine (15.3 mg, 0.082 mmol), and K₂CO₃ (26.3 mg, 0.19 mmol) in DMF (1.0 mL) was stirred at room temperature overnight. The reaction mixture was diluted with EtOAc and washed with water and sat. aq. NH₄Cl. The organic layers were dried over Na₂SO₄, filtered, and concentrated. The residue was purified by flash column chromatography (0% to 10% of DCM in MeOH) to afford G-2 (69 mg, 85% yield). ¹H NMR (600 MHZ, Chloroform-d) δ 8.35 (s, 1H), 8.11 (d, J=9.1 Hz, 1H), 7.64 (d, J=8.4 Hz, 2H), 7.41-7.34 (m, 2H), 7.34-7.21 (m, 5H), 7.06 (d, J=8.5 Hz, 1H), 6.99 (d, J=7.9 Hz, 2H), 6.76 (d, J=8.6 Hz, 2H), 6.61 (d, J=9.3 Hz, 1H), 3.95-3.83 (m, 1H), 3.71-3.49 (m, 8H), 3.45-3.30 (m, 5H), 3.30-3.20 (m, 4H), 3.20-3.13 (m, 2H), 3.10 (dd, J=13.9, 5.0 Hz, 1H), 3.02 (dd, J=13.9, 7.2 Hz, 1H), 2.89-2.75 (m, 2H), 2.61-2.47 (m, 5H), 2.47-2.07 (m, 19H), 1.92 (d, J=17.2 Hz, 1H), 1.71-1.56 (m, 2H), 1.52-1.39 (m, 10H), 0.95 (s, 3H). LC/MS (ESI) m/z 1284.4; [M+H]⁺ calcd for C₆₂H₈₂ClF₃N₉O₉S₃⁺: 1284.50.

Step 3: Synthesis of 4-(4-((4′-chloro-4-methyl-4-((4-(2-(piperazin-1-yl) acetyl) piperazin-1-yl)methyl)-3,4,5,6-tetrahydro-[1,1′-biphenyl]-2-yl)methyl) piperazin-1-yl)-N-((4-(((R)-4-morpholino-1-(phenylthio) butan-2-yl)amino)-3-12 ((trifluoromethyl) sulfonyl)phenyl) sulfonyl)benzamide hydrochloride (Intermediate G). HCl (1 mL, 4 N HCl in dioxane) was added to a stirring solution of G-2 (69 mg, 0.054 mmol) in DCM (1 mL) at room temperature. Then the resulting mixture was stirred at room temperature for 2 hours, then concentrated and dried under vacuum to afford Intermediate G (71 mg, quantitative yield), which was directly used in the next step. LC/MS (ESI) m/z 1184.5; [M+H]⁺ calcd for C₅₇H₇₄ClF₃N₉O₇S₃⁺: 1184.45.

### Preparation of Intermediates H, I, and J

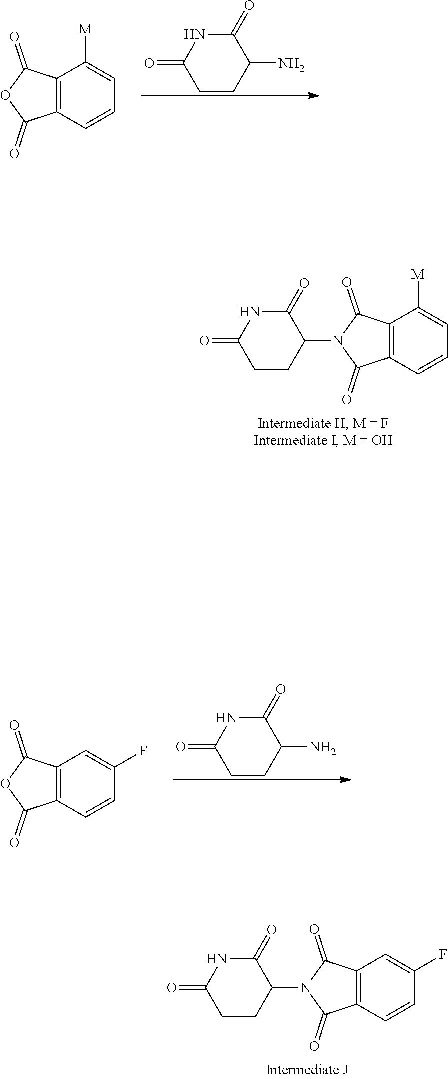

General synthetic procedure: A mixture of phthalic anhydride (1.0 equiv.), 3-aminopiperidine-2,6-dione hydrochloride (1.1 equiv.) and KOAc (1.3 equiv.) in acetic acid was stirred at 90° C. overnight. The resulting mixture was cooled to room temperature and concentrated. The residue was triturated with DCM and water, then collected the precipitate via filtration and dried under vacuum to afford Intermediates H, I and J.

2-(2,6-dioxopiperidin-3-yl)-4-fluoroisoindoline-1,3-dione (Intermediate H) (37.5 g, 90% 2 yield): ¹H NMR (600 MHz, DMSO-d₆) δ 11.15 (s, 1H), 7.99-7.92 (m, 1H), 7.80 (d, J=7.3 Hz, 1H), 7.74 (t, J=8.8 Hz, 1H), 5.17 (dd, J=12.9, 5.5 Hz, 1H), 2.90 (ddd, J=17.2, 13.9, 5.4 Hz, 1H), 2.62 (ddd, J=17.2, 4.7, 2.4 Hz, 1H), 2.58-2.47 (m, 1H), 2.13-2.03 (m, 1H). LC/MS (ESI) m/z 277.1; [M+H]⁺ calcd for C₁₃H₁₀FN₂O₄⁺: 277.06. 2-(2,6-dioxopiperidin-3-yl)-4-hydroxyisoindoline-1,3-dione (Intermediate I) (13.2 g, 79% yield): ¹H NMR (600 MHZ, DMSO-d₆) § 11.17 (s, 1H), 11.08 (s, 1H), 7.65 (dd, J=8.4, 7.2 Hz, 1H), 7.32 (d, J=7.1 Hz, 1H), 7.25 (d, J=8.3 Hz, 1H), 5.07 (dd, J=12.9, 5.4 Hz, 1H), 2.88 (ddd, J=17.0, 13.9, 5.4 Hz, 1H), 2.63-2.56 (m, 1H), 2.57-2.46 (m, 1H), 2.02 (dtd, J=13.1, 5.4, 2.3 Hz, 1H). LC/MS (ESI) m/z 275.1; [M+H]⁺ calcd for C₁₃H₁₁N₂O₅⁺: 275.07. 2-(2,6-dioxopiperidin-3-yl)-5-fluoroisoindoline-1,3-dione (Intermediate J) (27.6 g, 83% yield): ¹H NMR (600 MHZ, DMSO-d₆) δ 11.14 (s, 1H), 8.01 (dd, J=8.3, 4.4 Hz, 1H), 7.85 (dd, J=7.4, 2.3 Hz, 1H), 7.72 (ddd, J=9.4, 8.3, 2.3 Hz, 1H), 5.16 (dd, J=13.0, 5.4 Hz, 1H), 2.89 (ddd, J=17.2, 13.9, 5.4 Hz, 1H), 2.64-2.57 (m, 1H), 2.58 -2.46 (m, 1H), 2.12-2.02 (m, 1H). LC/MS (ESI) m/z 277.2; [M+H]⁺ calcd for C₁₃H₁₀FN₂O₄⁺: 277.06.

### Preparation of Intermediate K

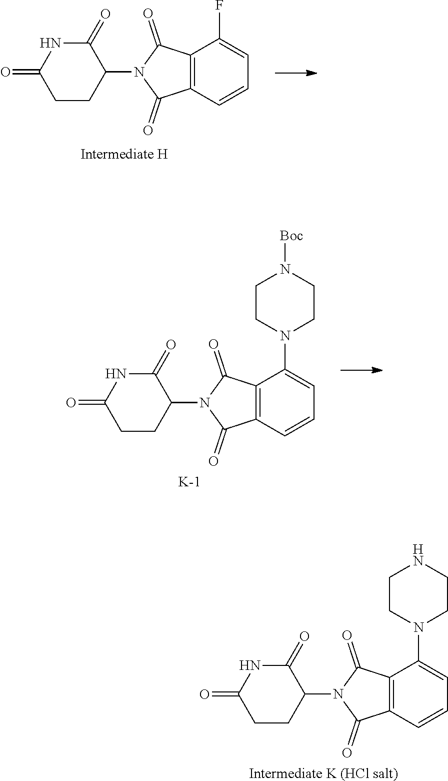

Step 1: Synthesis of tert-butyl 4-(2-(2,6-dioxopiperidin-3-yl)-1,3-dioxoisoindolin-4-22 yl) piperazine-1-carboxylate (K-1). A mixture of Intermediate H (300 mg, 1.09 mmol), N-Boc-piperazine (263 mg, 1.41 mmol), and DIPEA (567 μL, 3.26 mmol) in DMSO (5 mL) was stirred at 90° C. overnight. The reaction mixture was cooled to room temperature and diluted with EtOAc, then washed with water and sat. aq. NH₄Cl. The organic layers were dried over Na₂SO₄, filtered and concentrated. The residue was purified by flash column chromatography (30% to 100% of EtOAc in hexanes) to afford K-1 (320 mg, 63% yield). ¹H NMR (600 MHz, Chloroform-d) δ 8.05 (s, 1H), 7.63 (dd, J=8.4, 7.2 Hz, 1H), 7.46 (dd, J=7.2, 0.8 Hz, 1H), 7.19 (dd, J=8.5, 0.8 Hz, 1H), 4.99 (dd, J=12.6, 5.3 Hz, 1H), 3.68 (t, J=5.0 Hz, 4H), 3.36-3.25 (m, 4H), 2.99-2.68 (m, 3H), 2.18-2.11 (m, 1H), 1.51 (s, 9H). LC/MS (ESI) m/z 443.2; [M+H]⁺ calcd for C₂₂H₂₇N₄O₅⁺: 443.19.

2 Step 2: Synthesis of 2-(2,6-dioxopiperidin-3-yl)-4-(piperazin-1-yl) isoindoline-1,3-dione (Intermediate K): HCl (2 mL, 4 N HCl in dioxane) was added to a stirring solution of K-1 (150 mg, 3.39 mmol) in DCM (4 mL), then stirred at room temperature for 2 hours. The resulting mixture was concentrated and dried under vacuum to afford Intermediate K (151 mg, quantitative yield), which was directly used in the next step. LC/MS (ESI) m/z 343.1; [M+H]⁺ calcd for C₁₇H₁₉N₄O₄⁺: 343.14.

### Preparation of Intermediates L, M, N and P

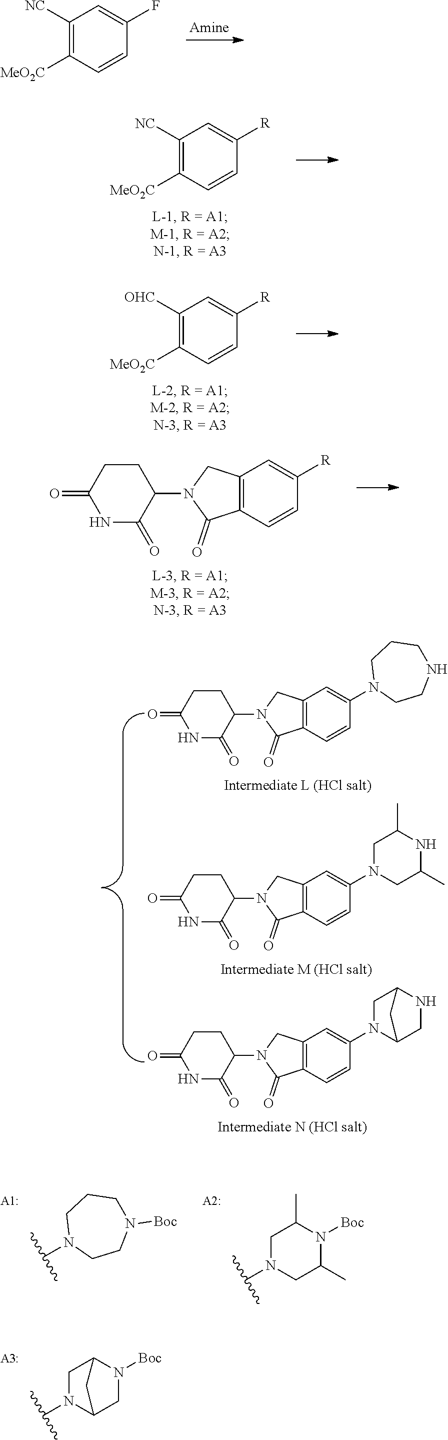

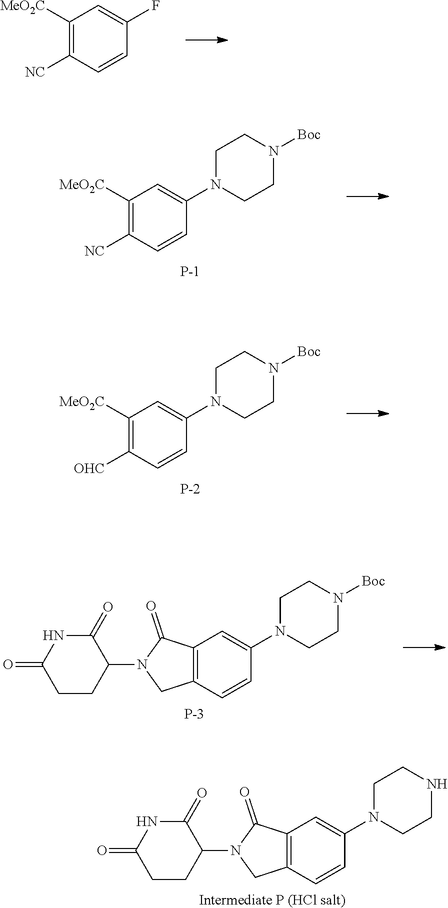

Step 1: General procedure for the synthesis of tert-butyl 4-(3-cyano-4-(methoxycarbonyl)phenyl)-1,4-diazepane-1-carboxylate (L-1), tert-butyl 4-(3-cyano-4-(methoxycarbonyl)phenyl)-1,4-diazepane-1-carboxylate (M-1), tert-butyl 5-(3-cyano-4-(methoxycarbonyl)phenyl)-2,5-diazabicyclo[2.2.1]heptane-2-carboxylate (N-1) and tert-butyl 4-(4-cyano-3-(methoxycarbonyl)phenyl) piperazine-1-carboxylate (P-1). A mixture of fluoride (1.0 equiv.), amine (1.1 equiv.), and DIPEA (3.0 equiv.) in DMSO was stirred at 110° C. for 2 hours. The resulting mixture was cooled to room temperature and diluted with EtOAc, then washed with water and sat. aq. NH₄Cl. The organic layers were dried over Na₂SO₄, filtered and concentrated. The residue was purified by flash column chromatography (0% to 60% of EtOAc in hexanes) to afford L-1, M-1, N-1, and P-1. L-1 (960 mg, 67% yield): ¹H NMR (600 MHZ, Chloroform-d) δ 7.96 (d, J=9.0 Hz, 1H), 6.98 (d, J=2.7 Hz, 1H), 6.83 (dd, J=9.0, 2.6 Hz, 1H), 3.92 (s, 3H), 3.67-3.56 (m, 6H), 3.35 (t, J=5.8 Hz, 1H), 3.25 (t, J=6.1 Hz, 1H), 2.00-1.91 (m, 2H), 1.38 (d, J=32.5 Hz, 9H). LC/MS (ESI) m/z 360.3; [M+H]⁺ calcd for C₁₉H₂₆N₃O₄⁺: 360.19. M-1 (610 mg, 64% yield): ¹H NMR (600 MHz, Chloroform-d) δ 7.99 (d, J=9.0 Hz, 1H), 7.13 (d, J=2.8 Hz, 1H), 6.98 (dd, J=9.0, 2.8 Hz, 1H), 4.34-4.26 (m, 2H), 3.94 (s, 3H), 3.65-3.57 (m, 2H), 3.16 (dd, J=12.7, 4.5 Hz, 2H), 1.49 (s, 9H), 1.30 (s, 3H), 1.29 (s, 3H). LC/MS (ESI) m/z 374.2; [M+H]⁺ calcd for C₂₀H₂₈N₃O₄⁺: 374.21. N-1:(700 mg, 70% yield): ¹H NMR (600 MHZ, Chloroform-d) δ 7.96 (d, J=8.9 Hz, 1H), 6.83 (d, J=8.6 Hz, 1H), 6.67 (dd, J=8.8, 2.6 Hz, 1H), 4.64 (d, J=80.9 Hz, 1H), 4.52-4.43 (m, 1H), 3.93 (s, 3H), 3.62-3.52 (m, 1H), 3.50-3.18 (m, 3H), 2.08-1.95 (m, 2H), 1.44 (d, J=29.7 Hz, 9H). LC/MS (ESI) m/z 358.2; [M+H]⁺ calcd for C₁₉H₂₄N₃O₄⁺: 358.18. P-1 (520 mg, 54% yield): ¹H NMR (599 MHz, Chloroform-d) δ 7.62 (d, J=8.7 Hz, 1H), 7.53 (d, J=2.8 Hz, 1H), 6.98 (dd, J=8.7, 2.8 Hz, 1H), 3.99 (s, 3H), 3.60 (t, J=5.3 Hz, 4H), 3.37 (t, J=5.2 Hz, 4H), 1.49 (s, 9H). LC/MS (ESI) m/z 346.1; [M+H]⁺ calcd for C₁₈H₂₄N₃O₄⁺: 346.18.

Step 2: General procedure for the synthesis of tert-butyl 4-(3-formyl-4-(methoxycarbonyl)phenyl)-1,4-diazepane-1-carboxylate (L-2), tert-butyl 4-(3-formyl-4-(methoxycarbonyl)phenyl)-2,6-dimethylpiperazine-1-carboxylate (M-2), tert-butyl 5-(3-formyl-4-(methoxycarbonyl)phenyl)-2,5-diazabicyclo[2.2.1]heptane-2-carboxylate (N-2) and tert-butyl 4-(4-formyl-3-(methoxycarbonyl)phenyl) piperazine-1-carboxylate (P-2). A mixture of L-1, M-1, N-1, or P-1 (1.0 equiv.), pyridine (30.0 equiv.), acetate acid (20.0 equiv.), Raney-Ni (2.0 equiv.) and NaH₂PO₂·H₂O (10.0 equiv.) in water was stirred at 85° C. overnight. The resulting mixture was cooled to room temperature and filtered through celite. The filtration was extracted with EtOAc. The combined organic layers were washed with water and sat. aq. NH₄Cl, dried over Na₂SO₄, then filtered and concentrated. The residue was purified by flash column chromatography (0% to 60% of EtOAc in hexanes) to afford L-2, M-2, N-2, and P-2. L-2 (567 mg, 59% yield): ¹H NMR (600 MHZ, Chloroform-d) & 10.75 (s, 1H), 7.91 (d, J=8.8 Hz, 1H), 7.15 (d, J=2.8 Hz, 1H), 6.82 (dd, J=8.9, 2.6 Hz, 1H), 3.90 (s, 3H), 3.72-3.55 (m, 6H), 3.33 (t, J=6.0 Hz, 1H), 3.22 (t, J=6.1 Hz, 1H), 2.02-1.93 (m, 2H), 1.38 (d, J=43.1 Hz, 9H). LC/MS (ESI) m/z 363.2; [M+H]⁺ calcd for C₁₉H₂₇N₂O₅⁺: 363.19. M-2 (345 mg, 56% yield): ¹H NMR (600 MHZ, Chloroform-d) δ 10.74 (s, 1H), 7.93 (d, J=8.8 Hz, 1H), 7.31 (d, J=2.8 Hz, 1H), 6.99 (dd, J=8.8, 2.8 Hz, 1H), 4.33-4.24 (m, 2H), 3.92 (s, 3H), 3.71-3.62 (m, 2H), 3.13 (dd, J=12.6, 4.5 Hz, 2H), 1.49 (s, 9H), 1.30 (s, 3H), 1.29 (s, 3H). LC/MS (ESI) m/z 377.2; [M+H]⁺ calcd for C₂₀H₂₉N₂O₅⁺: 377.21. N-2 (270 mg, 54% yield): ¹H NMR (600 MHZ, Chloroform-d) δ 10.74 (s, 1H), 7.92 (dd, J=8.8, 3.3 Hz, 1H), 6.98 (d, J=10.8 Hz, 1H), 6.65 (d, J=9.0 Hz, 1H), 4.75-4.46 (m, 2H), 3.91 (s, 3H), 3.59 (t, J=7.2 Hz, 1H), 3.48-3.21 (m, 3H), 2.03-1.93 (m, 2H), 1.43 (d, J=32.3 Hz, 9H). LC/MS (ESI) m/z 361.1; [M+H]⁺ calcd for C₁₉H₂₅N₂O₅⁺: 361.18. P-2 (198 mg, 44% yield): ¹H NMR (600 MHZ, Chloroform-d) δ 10.35 (s, 1H), 7.92 (d, J=8.8 Hz, 1H), 7.27 (d, J=2.7 Hz, 1H), 7.00 (dd, J=9.0, 2.6 Hz, 1H), 3.96 (s, 3H), 3.59 (t, J=5.3 Hz, 4H), 3.40 (t, J=5.3 Hz, 4H), 1.49 (s, 9H). LC/MS (ESI) m/z 349.2; [M+H]⁺ calcd for C₁₈H₂₅N₂O₅⁺: 349.18.

Step 3: General procedure for the synthesis of tert-butyl 4-(2-(2,6-dioxopiperidin-3-yl)-1-oxoisoindolin-5-yl)-1,4-diazepane-1-carboxylate (L-3), tert-butyl 4-(2-(2,6-dioxopiperidin-3-yl)-1-oxoisoindolin-5-yl)-2,6-dimethylpiperazine-1-carboxylate (M-3), tert-butyl 5-(2-(2,6-dioxopiperidin-3-yl)-1-oxoisoindolin-5-yl)-2,5-diazabicyclo[2.2.1]heptane-2-carboxylate (N-3) and tert-butyl 4-(2-(2,6-dioxopiperidin-3-yl)-3-oxoisoindolin-5-yl) piperazine-1-12 carboxylate (P-3). A mixture of L-2, M-2, N-2, or P-2 (1.0 equiv.), 3-aminopiperidine-2,6-dione hydrochloride (1.3 equiv.), acetate acid (1.2 equiv.) and NaBH(OAc)₃ (3.0 equiv.) in EtOH was stirred at 40° C. overnight. The resulting mixture was concentrated and the residue was purified by flash column chromatography (0% to 5% of MeOH in DCM) to afford L-3, M-3, N-3 and P-3.

L-3 (370 mg, 54% yield): ¹H NMR (600 MHZ, Chloroform-d) δ 8.16 (s, 1H), 7.70 (d, J=8.6 Hz, 1H), 6.78 (dd, J=8.7, 2.2 Hz, 1H), 6.70-6.62 (m, 1H), 5.21 (dd, J=13.3, 5.1 Hz, 1H), 4.39 (d, J=15.5 Hz, 1H), 4.26-4.21 (m, 1H), 3.71-3.52 (m, 6H), 3.40-3.27 (m, 1H), 3.23 (q, J=5.9 Hz, 1H), 2.95-2.87 (m, 1H), 2.83 (ddd, J=17.9, 13.2, 5.3 Hz, 1H), 2.35-2.27 (m, 1H), 2.21-2.16 (m, 1H), 2.03-1.91 (m, 2H), 1.39 (d, J=42.7 Hz, 9H). LC/MS (ESI) m/z 443.3; [M+H]⁺ 22 calcd for C₂₃H₃₁N₄O₅⁺: 443.23. M-3 (72 mg, 56% yield): ¹H NMR (600 MHZ, DMSO-d₆) δ 10.95 (s, 1H), 7.54 (d, J=8.5 Hz, 1H), 7.10 (d, J=2.1 Hz, 1H), 7.07 (dd, J=8.6, 2.2 Hz, 1H), 5.05 (dd, J=13.4, 5.1 Hz, 1H), 4.34 (d, J=16.7 Hz, 1H), 4.22 (d, J=16.8 Hz, 1H), 4.15-4.09 (m, 2H), 3.73 (d, J=12.7 Hz, 2H), 2.99 (dd, J=12.7, 4.4 Hz, 2H), 2.91 (ddd, J=17.3, 13.6, 5.4 Hz, 1H), 2.63-2.54 (m, 1H), 2.37 (qd, J=13.2, 4.5 Hz, 1H), 1.99-1.93 (m, 1H), 1.44 (s, 9H), 1.23 (s, 3H), 1.22 (s, 3H). LC/MS (ESI) m/z 457.2; [M+H]⁺ calcd for C₂₄H₃₃N₄O₅⁺: 457.24. M-3 (103 mg, 65% yield): ¹H NMR (600 MHZ, Chloroform-d) δ 8.07 (s, 1H), 7.74-7.67 (m, 1H), 6.67-6.57 (m, 1H), 6.55-6.47 (m, 1H), 5.25-5.16 (m, 1H), 4.71-4.43 (m, 2H), 4.39 (dd, J=15.5, 4.8 Hz, 1H), 4.28-4.19 (m, 1H), 3.65-3.56 (m, 1H), 3.50-3.35 (m, 2H), 3.24 (dd, J=50.7, 8.9 Hz, 1H), 2.94-2.87 (m, 1H), 2.87-2.78 (m, 1H), 2.31 (qd, J=13.2, 4.8 Hz, 1H), 2.23-2.16 (m, 1H), 2.04-1.93 (m, 2H), 1.43 (d, J=27.6 Hz, 9H). LC/MS (ESI) m/z 441.1; [M+H]⁺ calcd for C₂₃H₂₉N₄O₅⁺: 441.21. P-3 (70 mg, 33% yield): ¹H NMR (600 MHZ, Chloroform-d) δ 8.06 (s, 1H), 7.38 (d, J=2.4 Hz, 1H), 7.35 (d, J=8.4 Hz, 1H), 7.17 (dd, J=8.4, 2.4 Hz, 1H), 5.21 (dd, J=13.3, 5.1 Hz, 1H), 4.41 (d, J=15.5 Hz, 1H), 4.27 (d, J=15.7 Hz, 1H), 3.59 (t, J=5.2 Hz, 4H), 3.19 (t, J=5.2 Hz, 4H), 2.96-2.88 (m, 1H), 2.83 (ddd, J=18.0, 13.4, 5.4 Hz, 1H), 2.42-2.29 (m, 1H), 2.25-2.18 (m, 1H), 1.49 (s, 9H). LC/MS (ESI) m/z 429.2; [M+H]⁺ calcd for C C₂₂H₂₉N₄O₅⁺: 429.21.

Step 4: General procedure for the synthesis of 3-(5-(1,4-diazepan-1-yl)-1-oxoisoindolin-2-yl) piperidine-2,6-dione hydrochloride (intermediate L), 3-(5-(3,5-dimethylpiperazin-1-yl)-1-oxoisoindolin-2-yl) piperidine-2,6-dione hydrochloride (intermediate M), 3-(5-(2,5-diazabicyclo[2.2.1]heptan-2-yl)-1-oxoisoindolin-2-yl) piperidine-2,6-dione hydrochloride (intermediate N) and 3-(1-ox0-6-(piperazin-1-yl) isoindolin-2-yl) piperidine-2,6-dione hydrochloride (intermediate P). HCl (4 N HCl in dioxane) was added to a solution of L-3, M-3, N-3, or P-3 (1.0 equiv.) in DCM, then stirred at room temperature for 2 hours. The resulting mixture was concentrated to afford Intermediates L, M, N and P, which were directly used in the next step.

Intermediate L (362 mg, quantitative yield): LC/MS (ESI) m/z 343.1; [M+H]⁺ calcd for C₁₈H₂₃N₄O₃⁺: 343.18. Intermediate M (70 mg, quantitative yield): LC/MS (ESI) m/z 357.3; [M+H]⁺ calcd for C₁₉H₂₅N₄O₃⁺: 357.29. Intermediate N (100 mg, quantitative yield): LC/MS (ESI) m/z 341.2; [M+H]⁺ calcd for C₁₈H₂₁N₄O₃⁺: 341.16. Intermediate P (67 mg, quantitative yield): LC/MS (ESI) m/z 329.1; [M+H]⁺ calcd for C₁₇H₂₁N₄O₃⁺: 329.16.

### Preparation of Compounds in Table 1

### General Procedure A:

Step 1: A reaction mixture of Intermediate C (1.0 equiv.), Bromide (2.0 equiv.) and TEA (4.0 equiv.) in DCM were stirred at room temperature overnight. The mixture was washed with sat. aq. NHCl₄ and dried over Na₂SO₄, filtered and concentrated. The residue was purified by flash column chromatography (hexanes/EtOAc or DCM/MeOH) to afford a tert-butyl ester intermediate.

Step 2: To a solution of the tert-butyl ester intermediate from Step 1 (1.0 equiv.) in DCM was added HCl (4 N HCl in dioxane), then stirred at room temperature overnight. The reaction mixture was concentrated to afford a carboxylic acid intermediate, which was directly used in the next step.

Step 3: To a solution of the carboxylic acid intermediate from step 2 (1.2 equiv.) and HATU (1.5 equiv.) in DCM was added a solution of Intermediate D (1.0 equiv.) and TEA (6.0 equiv.) in DCM. The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH) to afford the desired compound.

### General Procedure B:

Step 1: Under an argon atmosphere, a mixture of Intermediate E (1.0 equiv.), alkyne (1.5 equiv.), Pd(Ph₃P)₂Cl₂ (0.15 equiv.) and CuI (0.3 equiv.) in TEA and DMSO were irradiated at 110° C. for 3 hours. The resulting mixture was diluted with EtOAc and washed with water and sat. aq. NHCl₄. The organic layers were dried over Na₂SO₄, filtered and concentrated. The residue was purified by flash column chromatography (hexanes/EtOAc or DCM/MeOH) to afford a Boc-protected amine.

Step 2: HCl (4 N HCl in dioxane) or TFA was added to a stirring solution of the Boc-protected amine from step 1 (1.0 equiv.) in DCM. Then the resulting mixture was stirred at room temperature for 1 hour. The reaction mixture was concentrated to afford the corresponding amine, which was directly used in the next step.

Step 3: Intermediate B (1.0 equiv.) and NaBH(OAc)₃ (1.2 equiv.) were added to a solution of the amine from step 2 (2.0 equiv.) and TEA (8.0 equiv.) in DCM, then stirred at room temperature overnight. The resulting mixture was washed with sat. aq. NH₄Cl. The organic layers were dried over Na₂SO₄, filtered and concentrated. The residue was purified by preparative TLC (DCM/MeOH) to afford the desired compound.

### General Procedure C:

Step 1: Aldehyde (1.2 equiv.) and NaBH(OAc)₃ (1.5 equiv.) were added to a solution of amine (1.0 equiv.) and TEA (6.0 equiv.) in DCM. The resulting mixture was stirred at room temperature overnight, then washed with water and sat. aq. NH₄Cl. The organic layers were dried over Na₂SO₄, filtered and concentrated. The residue was purified by flash column chromatography (hexanes/EtOAc or DCM/MeOH) to afford the reductive amination product.

Step 2:4 N HCl in dioxane or TFA was added to a stirring solution of the product from Step (1.0 equiv.) in DCM and stirred at room temperature for 1 hour. The reaction mixture was concentrated to afford the corresponding de-Boc product, which was directly used in the next step.

Step 3: Intermediate B (1.0 equiv.) and NaBH(OAc)₃ (1.2 equiv.) were added to a solution of the product from Step 2 (2.0 equiv.) and TEA (8.0 equiv.) in DCM, then stirred at room temperature overnight. The resulting mixture was washed with sat. aq. NH₄Cl. The organic layers were dried over Na₂SO₄, filtered and concentrated. The residue was purified by preparative TLC (DCM/MeOH) to afford the desired compound.

### General Procedure D:

Step 1: A stirring mixture of alkyne (1.0 equiv.) and 10% palladium on carbon (20%, w/w) in EtOAc/MeOH (v/v=1/1) was hydrogenated overnight with a hydrogen balloon. The resulting mixture was filtered through celite and the filtration was concentrated. The residue was purified by flash column chromatography (hexanes/EtOAc or DCM/MeOH) to afford the corresponding coupling product.

Step 2: To a solution of the product from Step 1 (1.2 equiv.) and HATU (1.5 equiv.) in DCM was added a solution of Intermediate D (1.0 equiv.) and TEA (6.0 equiv.) in DCM. The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH) to afford the desired compound.

### General Procedure E:

Step 1: A stirring mixture of alkyne (1.0 equiv.) and 10% palladium on carbon (20%, w/w) in EtOAc/MeOH (v/v=1/1) was hydrogenated overnight with a hydrogen balloon. The resulting mixture was filtered through celite and the filtration was concentrated. The residue was purified by flash column chromatography (hexanes/EtOAc or DCM/MeOH) to afford the corresponding coupling product.

Step 2:4 N HCl in dioxane or TFA was added to a stirring solution of the product from Step 1 (1.0 equiv.) in DCM and stirred at room temperature for 1 hour. The reaction mixture was concentrated to afford the corresponding de-Boc product, which was directly used in the next step.

Step 3: Intermediate B (1.0 equiv.) and NaBH(OAc)₃ (1.2 equiv.) were added to a solution of the product from Step 2 (2.0 equiv.) and TEA (8.0 equiv.) in DCM, then stirred at room temperature overnight. The resulting mixture was washed with sat. aq. NH₄Cl and dried over Na₂SO₄, filtered and concentrated. The residue was purified by preparative TLC (DCM/MeOH) to afford the desired compound.

### General Procedure F:

Step 1: A reaction mixture of Intermediate H (1.0 equiv.), amine (1.5 equiv.) and DIPEA (3.0 equiv.) in DMSO were stirred at 80° C. overnight. The resulting mixture was cooled to room temperature and diluted with EtOAc, then washed with water and sat. aq. NHCl₄. The organic layers were dried over Na₂SO₄, filtered and concentrated. The residue was purified by flash column chromatography (hexanes/EtOAc or DCM/MeOH) to afford a carboxylic acid intermediate.

Step 2: To a solution of the carboxylic acid intermediate from Step 1 (1.2 equiv.) and HATU (1.5 equiv.) in DCM was added a solution of Intermediate G (1.0 equiv.) and TEA (6.0 equiv.) in DCM. The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH) to afford the desired compound.

### General Procedure G:

Step 1: A reaction mixture of Intermediate H or I (1.0 equiv.), amine or bromide (1.2 equiv.) and DIPEA (3.0 equiv.) in NMP were irradiated at 110° C. for 3 hours. The resulting mixture was diluted with EtOAc and washed with water and sat. aq. NHCl₄. The organic layers were dried over Na₂SO₄, filtered and concentrated. The residue was purified by flash column chromatography (hexanes/EtOAc or DCM/MeOH) to afford the corresponding product.

Step 2: To a solution of the product from Step 1 (1.0 equiv.) in DCM was added HCl (4 N HCl in dioxane), then stirred at room temperature overnight. The reaction mixture was concentrated to afford a carboxylic acid intermediate, which was directly used in the next step.

Step 3: To a solution of 19-3 or the carboxylic acid intermediate from Step 2 (1.2 equiv.) and HATU (1.5 equiv.) in DCM was added a solution of Intermediate G, 19-2, 20-2, or 21-2 (1.0 equiv.) and TEA (6.0 equiv.) in DCM. The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH) to afford the desired compound.

### General Procedure H:

Intermediate B (1.0 equiv.) and NaBH(OAc)₃ (1.2 equiv.) were added to a solution of an appropriate amine (2.0 equiv.) and TEA (8.0 equiv.) in DCM, then stirred at room temperature overnight. The resulting mixture was washed with sat. aq. NH₄Cl. The organic layers were dried over Na₂SO₄, filtered and concentrated. The residue was purified by preparative TLC (DCM/MeOH) to afford the desired compound.

### Example 1: Preparation of Compound #11

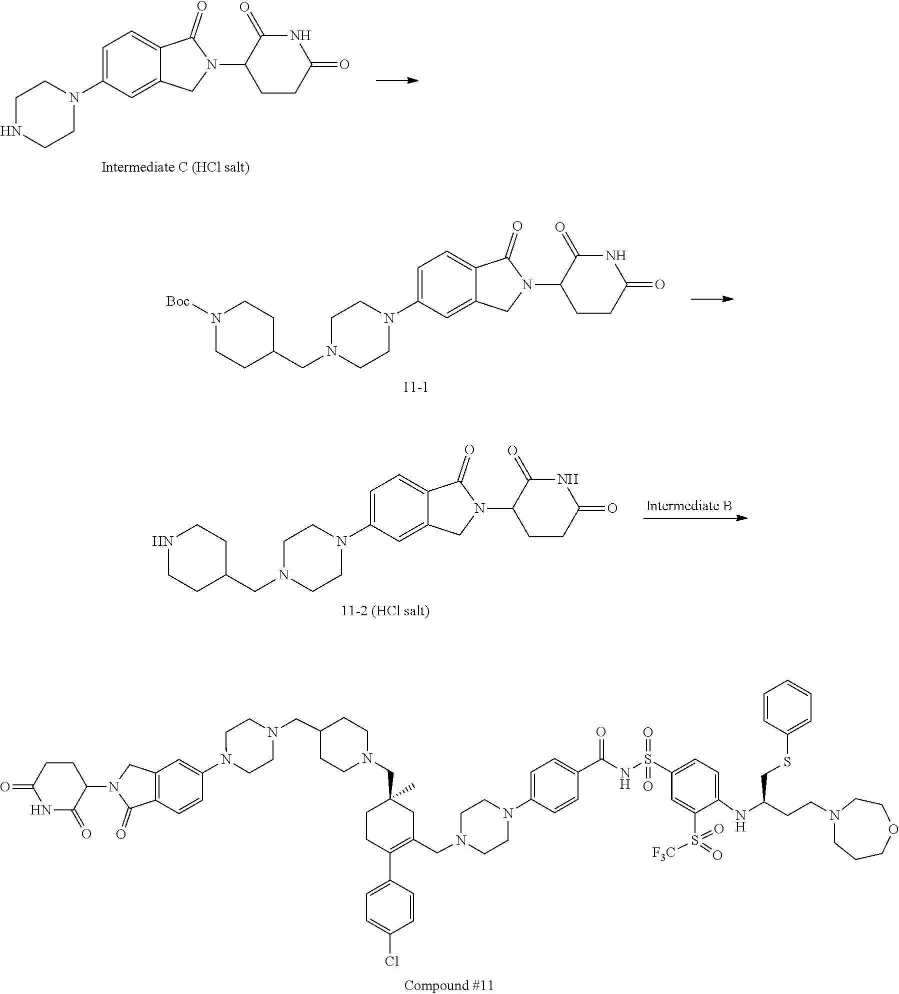 [SMILES: O=C1CCC(N2Cc3cc(N4CCNCC4)ccc3C2=O)C(=O)N1 ; CC(C)(C)OC(=O)N1CCC(CN2CCN(c3ccc4c(c3)CN(C3CCC(=O)NC3=O)C4=O)CC2)CC1 ; O=C1CCC(N2Cc3cc(N4CCN(CC5CCNCC5)CC4)ccc3C2=O)C(=O)N1 ; C[C@@]1(CN2CCC(CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Step 1: Synthesis of tert-butyl 4-((4-(2-(2,6-dioxopiperidin-3-yl)-1-oxoisoindolin-5-yl) piperazin-1-yl)methyl) piperidine-1-carboxylate (11-1). 1-Boc-piperidine-4-carboxaldehyde (93 mg, 0.44 mmol) and NaBH(OAc)₃ (92 mg, 0.44 mmol) were added to a solution of Intermediate C (106 mg, 0.29 mmol) and TEA (242 μL, 1.74 mmol) in DCM (5 mL). The resulting mixture was stirred at room temperature overnight, then washed with water and sat. aq. NH₄Cl. The organic layers were dried over Na₂SO₄, filtered and concentrated. The residue was purified by flash column chromatography (0% to 15% of MeOH in DCM) to afford 11-1 (112 mg, 74% yield) as an off-white solid. ¹H NMR (600 MHZ, Chloroform-d) δ 8.04 (s, 1H), 7.75 (d, J=8.6 Hz, 1H), 7.01 (dd, J=8.6, 2.2 Hz, 1H), 6.89 (d, J=2.2 Hz, 1H), 5.22 (dd, J=13.3, 5.1 Hz, 1H), 4.43 (d, J=15.6 Hz, 1H), 4.27 (d, J=15.6 Hz, 1H), 4.20-4.02 (m, 2H), 3.37-3.28 (m, 4H), 2.98-2.79 (m, 2H), 2.78-2.65 (m, 2H), 2.58 (t, J=5.1 Hz, 4H), 2.34 (qd, J=13.2, 4.8 Hz, 1H), 2.25 (d, J=7.2 Hz, 2H), 2.24-2.19 (m, 1H), 1.78 (d, J=13.2 Hz, 2H), 1.73-1.66 (m, 1H), 1.48 (s, 9H), 1.12 (qd, J=12.3, 4.3 Hz, 2H). LC/MS (ESI) m/z 526.1; [M+H]⁺ calcd for C₂₈H₄₀N₅O₅⁺: 526.3.

Step 2: Synthesis of 3-(1-oxo-5-(4-(piperidin-4-ylmethyl) piperazin-1-yl) isoindolin-2-28 yl) piperidine-2,6-dione (11-2). HCl (1 mL, 4 N HCl in dioxane) was added to a stirring solution of 11-1 (70 mg, 0.13 mmol) in DCM (1 mL) at room temperature. Then the resulting mixture was stirred at room temperature for 1 hour, the reaction mixture was concentrated to afford 11-2 (72 mg, quantitative yield) as HCl salt, which was used in the next step directly. LC/MS (ESI) m/z 426.2; [M+H]⁺ calcd for C₂₃H₃₂N₅O₃⁺: 426.25.

Step 3: Synthesis of N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-(2-(2,6-dioxopiperidin-3-yl)-1-oxoisoindolin-5-yl) piperazin-1-yl)methyl) piperidin-1-yl)methyl)-4-methyl-3,4,5,6-tetrahydro-[1,1′-biphenyl]-2-yl)methyl) piperazin-1-yl)benzamide (compound #11). Intermediate B (15 mg, 0.015 mmol) and NaBH(OAc)₃ (3 mg, 0.023 mmol) were added to a solution of 11-2 (9 mg, 0.0195 mmol) and TEA (13 μL, 0.09 mmol) in DCM (1 mL), then stirred at room temperature overnight. The resulting mixture was washed with sat. aq. NH₄Cl. The organic layers were dried over Na₂SO₄, filtered and concentrated. The residue was purified by preparative TLC (DCM/MeOH=10/1) to afford compound #11 (5.37 mg, 25% yield) as an off-white solid. ¹H NMR (600 MHZ, Chloroform-d) δ 8.32 (d, J=2.3 Hz, 1H), 8.01 (d, 1H), 7.84 (d, J=8.5 Hz, 2H), 7.71 (d, J=8.6 Hz, 1H), 7.36 (d, J=7.6 Hz, 2H), 7.32-7.25 (m, 4H), 7.23 (t, J=7.5 Hz, 1H), 6.97 (t, J=8.8 Hz, 3H), 6.86 (s, 1H), 6.74 (d, J=8.6 Hz, 2H), 6.47 (d, J=9.2 Hz, 1H), 5.16 (dd, J=13.4, 5.0 Hz, 1H), 4.40 (d, J=15.8 Hz, 1H), 4.26 (d, J=15.8 Hz, 1H), 3.85-3.67 (m, 5H), 3.28 (t, J=5.0 Hz, 4H), 3.25-3.14 (m, 5H), 3.09 (dd, J=13.8, 5.0 Hz, 2H), 3.02 (dd, J=13.9, 6.9 Hz, 1H), 2.92-2.64 (m, 7H), 2.63-2.42 (m, 7H), 2.41-2.21 (m, 9H), 2.21-2.14 (m, 1H), 2.14-2.01 (m, 2H), 1.90 (p, J=6.0 Hz, 2H), 1.85-1.53 (m, 14H), 1.53-1.41 (m, 1H), 1.02 (s, 3H) . . . . LC/MS (ESI) m/z 1411.7; [M+H]⁺ calcd for C₇₁H₈₇ClF₃N₁₀O₃S₃⁺: 1411.55.

### Example 2: Preparation of Compounds #1-3

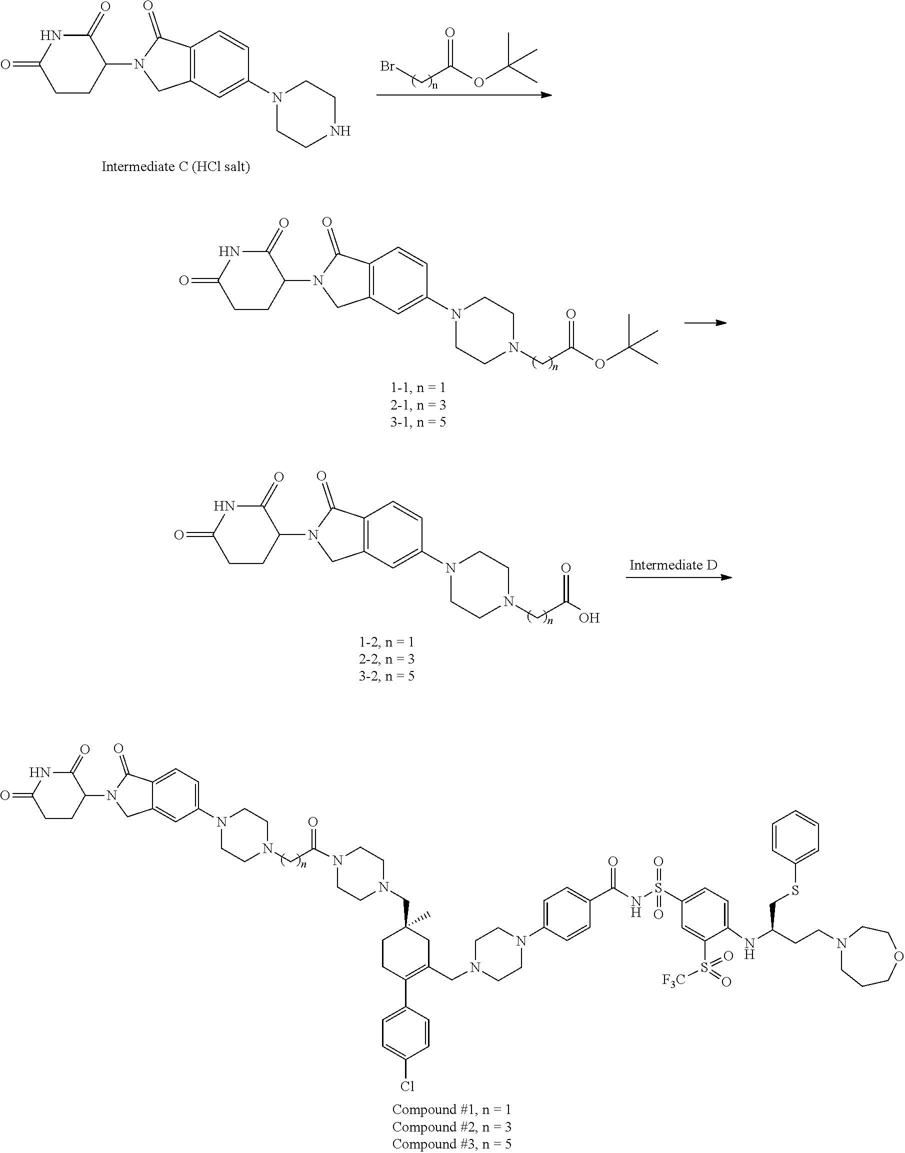

Compounds #1-3 were Prepared by Following General Procedure A:

tert-butyl 2-(4-(2-(2,6-dioxopiperidin-3-yl)-1-oxoisoindolin-5-yl) piperazin-1-yl)acetate (1-1) (59 mg, 81% yield). ¹H NMR (600 MHZ, Chloroform-d) δ 8.04 (s, 1H), 7.75 (d, J=8.6 Hz, 1H), 7.02 (dd, J=8.6, 2.2 Hz, 1H), 6.90 (s, 1H), 5.22 (dd, J=13.3, 5.1 Hz, 1H), 4.43 (d, J=15.6 Hz, 1H), 4.28 (d, J=15.6 Hz, 1H), 3.42-3.37 (m, 4H), 3.21 (s, 2H), 2.97-2.89 (m, 1H), 2.88-2.80 (m, 1H), 2.80-2.74 (m, 4H), 2.37-2.29 (m, 1H), 2.26-2.18 (m, 1H), 1.50 (s, 9H). LC/MS (ESI) m/z 443.4; [M+H]⁺ calcd for C₂₃H₃₁N₄O₅⁺: 443.52.

tert-butyl 4-(4-(2-(2,6-dioxopiperidin-3-yl)-1-oxoisoindolin-5-yl) piperazin-1-yl) butanoate (2-1) (66 mg, 61% yield). ¹H NMR (600 MHZ, Chloroform-d) δ 8.11 (s, 1H), 7.75 (d, J=8.6 Hz, 1H), 7.01 (dd, J=8.6, 2.2 Hz, 1H), 6.90 (d, J=2.1 Hz, 1H), 5.22 (dd, J=13.3, 5.1 Hz, 1H), 4.43 (d, J=15.6 Hz, 1H), 4.28 (d, J=15.6 Hz, 1H), 3.35 (t, J=5.1 Hz, 4H), 2.92 (ddd, J=17.8, 4.8, 2.6 Hz, 1H), 2.84 (ddd, J=18.0, 13.3, 5.3 Hz, 1H), 2.64 (t, J=5.0 Hz, 4H), 2.45 (t, J=7.5 Hz, 2H), 2.39-2.28 (m, 3H), 2.25-2.18 (m, 1H), 1.84 (p, J=7.5 Hz, 2H), 1.47 (s, 9H). LC/MS (ESI) m/z 471.2; [M+H]⁺ calcd for C₂₅H₃₅N₄O₅⁺: 471.26.

tert-butyl 6-(4-(2-(2,6-dioxopiperidin-3-yl)-1-oxoisoindolin-5-yl) piperazin-1-yl) hexanoate (3-1) (84 mg, 68% yield). ¹H NMR (600 MHZ, Chloroform-d) δ 8.08 (s, 1H), 7.75 (d, J=8.6 Hz, 1H), 7.01 (dd, J=8.6, 2.2 Hz, 1H), 6.90 (d, J=2.1 Hz, 1H), 5.22 (dd, J=13.3, 5.1 Hz, 1H), 4.43 (d, J=15.6 Hz, 1H), 4.28 (d, J=15.6 Hz, 1H), 3.35 (t, J=5.1 Hz, 4H), 2.96-2.88 (m, 1H), 2.88-2.79 (m, 1H), 2.62 (t, J=4.9 Hz, 4H), 2.42 (t, J=7.7 Hz, 2H), 2.38-2.29 (m, 1H), 2.28-2.17 (m, 3H), 1.64 (p, J=7.5 Hz, 2H), 1.60-1.53 (m, 2H), 1.47 (s, 9H), 1.43-1.34 (m, 2H). LC/MS (ESI) m/z 499.2; [M+H]⁺ calcd for C₂₇H₃₉N₄O₅⁺: 499.29.

2-(4-(2-(2,6-dioxopiperidin-3-yl)-1-oxoisoindolin-5-yl) piperazin-1-yl) acetic acid (1-2) (1-1 mg, quantitative yield). LC/MS (ESI) m/z 387.1; [M+H]⁺ calcd for C₁₉H₂₃N₄O₅⁺: 387.17.

4-(4-(2-(2,6-dioxopiperidin-3-yl)-1-oxoisoindolin-5-yl) piperazin-1-yl) butanoic acid (2-2) (13 mg, quantitative yield). LC/MS (ESI) m/z 415.1; [M+H]⁺ calcd for C₂₁H₂₇N₄O₅⁺: 415.20.

6-(4-(2-(2,6-dioxopiperidin-3-yl)-1-oxoisoindolin-5-yl) piperazin-1-yl) hexanoic acid (3-2) (13 mg, quantitative yield). LC/MS (ESI) m/z 443.1; [M+H]⁺ calcd for C₂₃H₃₁N₄O₅⁺: 443.23.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-(2-(2,6-dioxopiperidin-3-yl)-1-oxoisoindolin-5-yl) piperazin-1-yl) acetyl) piperazin-1-yl)methyl)-4-methyl-3,4,5,6-tetrahydro-[1,1′-biphenyl]-2-yl)methyl) piperazin-1-yl)benzamide (compound #1) (2.83 mg, 15% yield). ¹H NMR (600 MHZ, Chloroform-d) δ 8.31 (s, 1H), 8.07 (d, J=9.1 Hz, 1H), 7.74 (d, J=8.4 Hz, 2H), 7.70 (d, J=8.7 Hz, 1H), 7.40-7.35 (m, 3H), 7.34-7.21 (m, 5H), 7.02-6.94 (m, 3H), 6.88 (s, 1H), 6.75 (d, J=8.4 Hz, 2H), 6.59 (d, J=9.3 Hz, 1H), 5.18 (d, J=13.1 Hz, 1H), 4.43-4.36 (m, 1H), 4.27 (d, J=15.8 Hz, 1H), 3.89 (s, 1H), 3.76 (t, J=6.1 Hz, 2H), 3.74-3.66 (m, 2H), 3.65-3.54 (m, 4H), 3.30 (s, 4H), 3.28-3.16 (m, 6H), 3.11 (dd, J=14.1, 5.0 Hz, 1H), 3.04 (dd, J=14.0, 6.8 Hz, 1H), 2.97-2.79 (m, 4H), 2.78-2.50 (m, 13H), 2.43-2.14 (m, 12H), 2.12-2.01 (m, 1H), 1.96-1.85 (m, 3H), 1.76-1.68 (m, 1H), 1.66-1.58 (m, 1H), 1.49-1.43 (m, 1H), 0.97 (s, 3H). LC/MS (ESI) m/z 1440.5; [M+H]⁺ calcd for C₇₁H₈₆ClF₃N₁₁O₁₀S₃⁺: 1440.54.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(4-(4-(2-(2,6-dioxopiperidin-3-yl)-1-oxoisoindolin-5-yl) piperazin-1-yl) butanoyl) piperazin-1-yl)methyl)-4-methyl-3,4,5,6-tetrahydro-[1,1′-biphenyl]-2-yl)methyl) piperazin-1-yl)benzamide (compound #2) (2.79 mg, 14% yield). ¹H NMR (600 MHZ, Chloroform-d) δ 8.38 (d, J=3.0 Hz, 1H), 8.05 (d, J=8.5 Hz, 1H), 7.84 (d, J=8.5 Hz, 2H), 7.77-7.69 (m, 1H), 7.39 (d, J=7.3 Hz, 2H), 7.34-7.23 (m, 6H), 7.08-6.97 (m, 4H), 6.93 (s, 1H), 6.77 (dd, J=8.7, 4.6 Hz, 2H), 6.55 (d, J=9.3 Hz, 1H), 5.23 (d, J=12.7 Hz, 1H), 4.42 (d, J=15.7 Hz, 1H), 4.29 (d, J=15.8 Hz, 1H), 3.88 (s, 1H), 3.84-3.66 (m, 4H), 3.50-3.28 (m, 8H), 3.23 (s, 4H), 3.10 (dd, J=13.8, 5.0 Hz, 1H), 3.02 (dd, J=13.8, 7.1 Hz, 1H), 2.96-2.83 (m, 8H), 2.81-2.51 (m, 9H), 2.51-2.24 (m, 12H), 2.25-2.05 (m, 5H), 1.95-1.84 (m, 4H), 1.76-1.69 (m, 1H), 1.69-1.62 (m, 1H), 1.62-1.55 (m, 1H), 1.51-1.43 (m, 1H), 0.98 (s, 3H). LC/MS (ESI) m/z 1468.5; [M+H]⁺ calcd for C₇₃H₉₀ClF₃N₁₁O₁₀S₃⁺: 1468.57.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(6-(4-(2-(2,6-dioxopiperidin-3-yl)-1-oxoisoindolin-5-yl) piperazin-1-yl) hexanoyl) piperazin-1-yl)methyl)-4-methyl-3,4,5,6-tetrahydro-[1,1′-biphenyl]-2-yl)methyl) piperazin-1-yl)benzamide (compound #3) (1.13 mg, 6% yield). ¹H NMR (600 MHZ, Chloroform-d) δ 8.38 (s, 1H), 8.07 (d, J=9.2 Hz, 1H), 7.79 (d, J=8.7 Hz, 2H), 7.75 (d, J=8.6 Hz, 1H), 7.42-7.36 (m, 2H), 7.34-7.23 (m, 6H), 7.10 (d, J=8.4 Hz, 1H), 7.01 (d, 3H), 6.94 (d, J=5.2 Hz, 1H), 6.79 (d, J=8.7 Hz, 2H), 6.58 (d, J=9.3 Hz, 1H), 5.24 (dd, J=13.7, 4.1 Hz, 1H), 4.43 (d, J=15.7 Hz, 1H), 4.29 (d, J=15.8 Hz, 1H), 3.90 (s, 1H), 3.77 (t, J=6.1 Hz, 2H), 3.75-3.64 (m, 4H), 3.48 (s, 4H), 3.43-3.32 (m, 2H), 3.23 (s, 4H), 3.10 (dd, J=13.8, 5.1 Hz, 1H), 3.03 (dd, J=14.0, 7.1 Hz, 1H), 2.96-2.76 (m, 8H), 2.76-2.53 (m, 8H), 2.53-2.17 (m, 17H), 2.12-2.05 (m, 1H), 1.91-1.84 (m, 4H), 1.79-1.56 (m, 7H), 1.51-1.45 (m, 1H), 0.99 (s, 3H). LC/MS (ESI) m/z 1496.7; [M+H]⁺ calcd for C₇₅H₉₄ClF₃N₁₁O₁₀S₃⁺: 1496.60.

### Example 3: Preparation of Compound #4

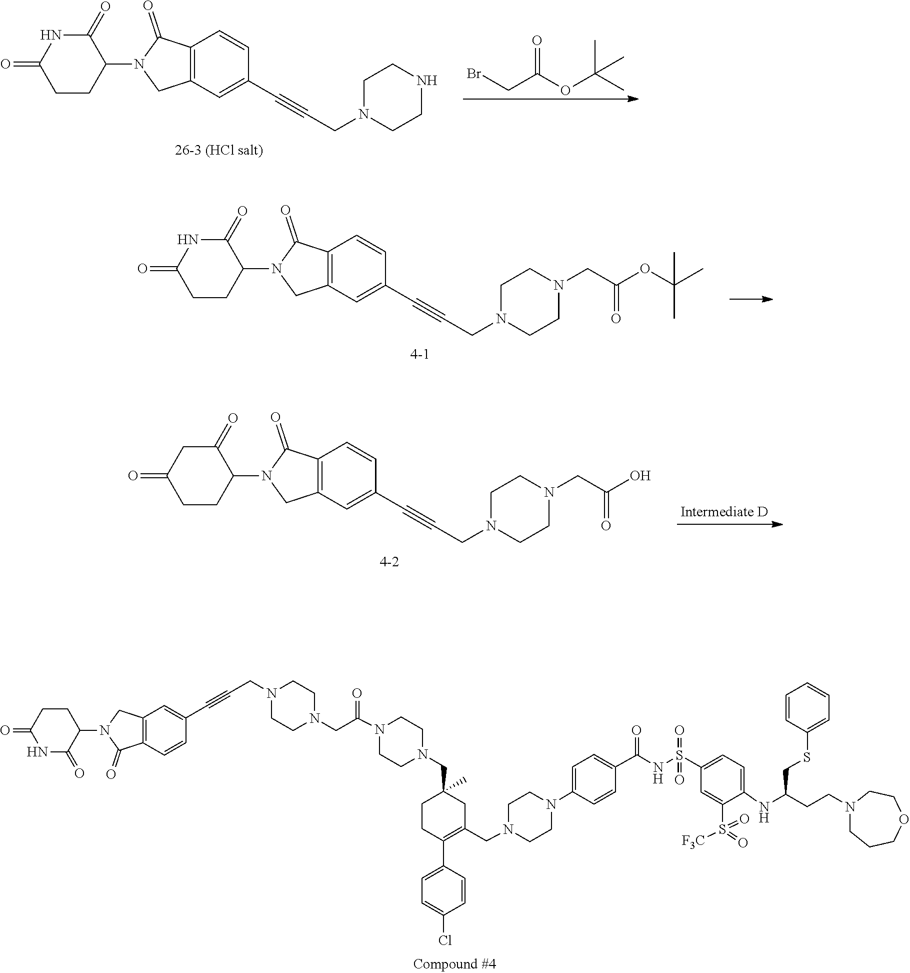

### Compound #4 was Prepared by Following General Procedure A:

tert-butyl 2-(4-(3-(2-(2,6-dioxopiperidin-3-yl)-1-oxoisoindolin-5-yl) prop-2-yn-1-yl) piperazin-1-yl)acetate (4-1) (11 mg, 21% yield). ¹H NMR (600 MHZ, Chloroform-d) δ 8.24 (s, 1H), 7.83 (d, J=7.8 Hz, 1H), 7.56 (dd, J=7.9, 1.3 Hz, 1H), 7.54 (s, 1H), 5.23 (dd, J=13.4, 5.1 Hz, 1H), 4.49 (d, J=16.0 Hz, 1H), 4.33 (d, J=16.0 Hz, 1H), 3.58 (s, 2H), 3.15 (s, 2H), 2.95 (ddd, J=17.8, 4.8, 2.5 Hz, 1H), 2.85 (ddd, J=17.9, 13.4, 5.4 Hz, 1H), 2.76 (s, 4H), 2.69 (s, 4H), 2.38 (qd, J=13.3, 4.7 Hz, 1H), 2.29-2.20 (m, 1H), 1.49 (s, 9H). LC/MS (ESI) m/z 481.1; [M+H]⁺ calcd for C₂₆H₃₃N₄O₅⁺: 481.24.

2-(4-(3-(2-(2,6-dioxopiperidin-3-yl)-1-oxoisoindolin-5-yl) prop-2-yn-1-yl) piperazin-1-yl) acetic acid (4-2) (10 mg, quantitative yield). LC/MS (ESI) m/z 424.2; [M+H]⁺ calcd for C₂₂H₂₄N₄O₅⁺: 424.17.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-(3-(2-(2,6-dioxopiperidin-3-yl)-1-oxoisoindolin-5-yl) prop-2-yn-1-yl) piperazin-1-yl) acetyl) piperazin-1-yl)methyl)-4-methyl-3,4,5,6-tetrahydro-[1,1′-biphenyl]-2-yl)methyl) piperazin-1-yl)benzamide (compound #4) (8.27 mg, 41% yield). ¹H NMR (600 MHZ, Chloroform-d) δ 8.32 (s, 1H), 8.07 (d, J=9.1 Hz, 1H), 7.81-7.76 (m, 3H), 7.56-7.51 (m, 2H), 7.38 (d, J=7.4 Hz, 2H), 7.33-7.22 (m, 6H), 7.09-7.06 (m, 1H), 7.04-6.98 (m, 2H), 6.77 (d, J=8.6 Hz, 2H), 6.59 (d, J=9.3 Hz, 1H), 5.25 (dd, J=13.4, 4.6 Hz, 1H), 4.46 (d, J=16.1 Hz, 1H), 4.33 (d, J=16.2 Hz, 1H), 3.90 (s, 1H), 3.77 (t, J=6.1 Hz, 2H), 3.75-3.68 (m, 2H), 3.67-3.52 (m, 6H), 3.25 (s, 4H), 3.21 (s, 2H), 3.11 (dd, J=13.9, 5.0 Hz, 1H), 3.04 (dd, J=13.9, 6.9 Hz, 1H), 2.95-2.83 (m, 4H), 2.83-2.48 (m, 16H), 2.47-2.27 (m, 6H), 2.27-2.04 (m, 8H), 2.01-1.86 (m, 3H), 1.82-1.69 (m, 1H), 1.67-1.56 (m, 1H), 1.52-1.42 (m, 1H), 0.97 (s, 3H). LC/MS (ESI) m/z 1478.4; [M+H]⁺ calcd for C₇₄H₈₈ClF₃N₁₁O₁₀S₃⁺: 1478.55.

### Example 4: Preparation of Compound #5

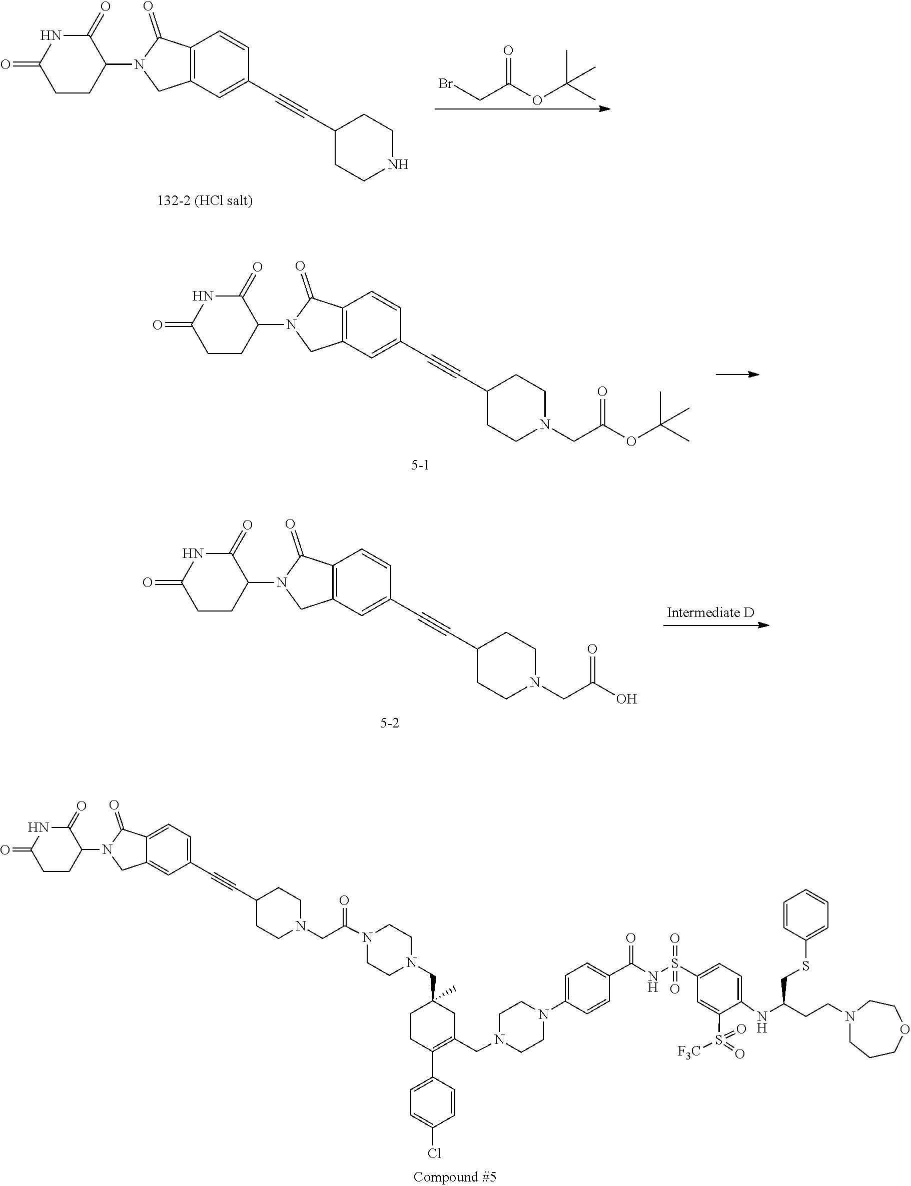

### Compound #5 was Prepared by Following General Procedure A:

tert-butyl 2-(4-((2-(2,6-dioxopiperidin-3-yl)-1-oxoisoindolin-5-yl) ethynyl) piperidin-1-yl)acetate (5-1) (32 mg, 62% yield). ¹H NMR (600 MHZ, Chloroform-d) δ 8.36 (s, 1H), 7.81 (d, J=7.9 Hz, 1H), 7.52 (dd, J=7.8, 1.3 Hz, 1H), 7.50 (s, 1H), 5.23 (dd, J=13.3, 5.1 Hz, 1H), 4.48 (d, J=15.9 Hz, 1H), 4.32 (d, J=15.9 Hz, 1H), 3.15 (s, 2H), 2.97-2.90 (m, 1H), 2.89-2.80 (m, 3H), 2.71-2.65 (m, 1H), 2.45 (t, J=10.1 Hz, 2H), 2.37 (qd, J=13.2, 4.7 Hz, 1H), 2.28-2.20 (m, 1H), 2.03-1.94 (m, 2H), 1.90-1.79 (m, 2H), 1.48 (s, 9H). LC/MS (ESI) m/z 466.2; [M+H]⁺ calcd for C₂₆H₃₂N₃O₅⁺: 466.23.

2-(4-((2-(2,6-dioxopiperidin-3-yl)-1-oxoisoindolin-5-yl) ethynyl) piperidin-1-yl) acetic acid (5-2) (11 mg, quantitative yield). LC/MS (ESI) m/z 410.1; [M+H]⁺ calcd for C₂₂H₂₄N₃O₅⁺: 410.17.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-((2-(2,6-dioxopiperidin-3-yl)-1-oxoisoindolin-5-yl) ethynyl) piperidin-1-yl) acetyl) piperazin-1-yl)methyl)-4-methyl-3,4,5,6-tetrahydro-[1,1′-biphenyl]-2-yl)methyl) piperazin-1-yl)benzamide (compound #5) (7.07 mg, 36% yield). ¹H NMR (600 MHz, Chloroform-d) δ 8.51 (s, 1H), 8.33 (t, J=2.2 Hz, 1H), 8.07 (dt, J=9.4, 2.7 Hz, 1H), 7.84-7.74 (m, 3H), 7.54-7.47 (m, 2H), 7.41-7.35 (m, 2H), 7.33-7.27 (m, 4H), 7.27-7.22 (m, 1H), 7.07 (d, J=8.5 Hz, 1H), 7.03-6.98 (m, 2H), 6.76 (d, J=8.7 Hz, 2H), 6.59 (d, J=9.3 Hz, 1H), 5.29 (ddd, J=16.0, 13.3, 5.1 Hz, 1H), 4.47 (d, J=16.1 Hz, 1H), 4.33 (d, J=16.1 Hz, 1H), 3.90 (d, J=7.9 Hz, 1H), 3.80-3.75 (m, 2H), 3.75-3.68 (m, 2H), 3.60 (s, 4H), 3.35-3.17 (m, 6H), 3.11 (dd, J=13.9, 5.0 Hz, 1H), 3.04 (dd, J=13.9, 6.8 Hz, 1H), 2.96-2.75 (m, 10H), 2.75-2.49 (m, 8H), 2.45-2.21 (m, 12H), 2.17 (d, J=17.4 Hz, 1H), 2.13-2.05 (m, 1H), 2.04-1.98 (m, 2H), 1.97-1.87 (m, 3H), 1.85-1.71 (m, 2H), 1.70-1.57 (m, 1H), 1.53-1.44 (m, 1H), 0.98 (s, 3H). LC/MS (ESI) m/z 1463.4; [M+H]⁺ calcd for C₇₄H₈₇ClF₃N₁₀O₁₀S₃⁺: 1463.54.

### Example 5: Preparation of Compound #6

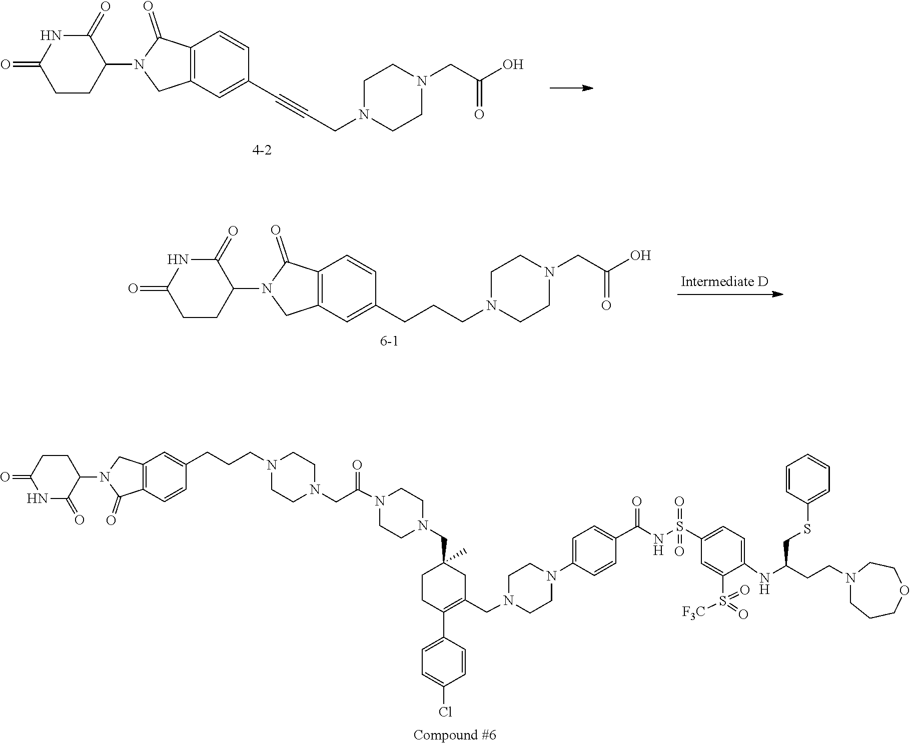 [SMILES: O=C(O)CN1CCN(CC#Cc2ccc3c(c2)CN(C2CCC(=O)NC2=O)C3=O)CC1 ; O=C(O)CN1CCN(CCCc2ccc3c(c2)CN(C2CCC(=O)NC2=O)C3=O)CC1 ; C[C@@]1(CN2CCN(C(=O)CN3CCN(CCCc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #6 was Prepared by Following General Procedure D:

2-(4-(3-(2-(2,6-dioxopiperidin-3-yl)-1-oxoisoindolin-5-yl) propyl) piperazin-1-yl) acetic acid (6-1) (17 mg, 89% yield). LC/MS (ESI) m/z 429.2; [M+H]⁺ calcd for C₂₂H₂₉N₄O₅⁺: 429.21.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-(3-(2-(2,6-dioxopiperidin-3-yl)-1-oxoisoindolin-5-yl) propyl) piperazin-1-yl) acetyl) piperazin-1-yl)methyl)-4-methyl-3,4,5,6-tetrahydro-[1,1′-biphenyl]-2-yl)methyl) piperazin-1-yl)benzamide (compound #6) (5.28 mg, 26% yield). ¹H NMR (600 MHZ, Chloroform-d) δ 8.38 (s, 1H), 8.04 (dd, J=9.2, 2.2 Hz, 1H), 7.89 (d, 2H), 7.77 (dd, J=7.8, 2.8 Hz, 1H), 7.41-7.37 (m, 2H), 7.35-7.22 (m, 7H), 7.05-6.95 (m, 3H), 6.79 (d, J=8.6 Hz, 2H), 6.56 (d, J=9.6, 1.7 Hz, 1H), 5.21 (ddd, J=13.3, 5.2, 1.6 Hz, 1H), 4.46-4.24 (m, 2H), 3.87 (s, 1H), 3.77 (t, J=6.1 Hz, 2H), 3.74-3.66 (m, 2H), 3.48-3.28 (m, 3H), 3.20 (s, 4H), 3.10 (dd, J=14.0, 4.3 Hz, 1H), 3.06-2.96 (m, 3H), 2.96-2.87 (m, 3H), 2.87-2.53 (m, 16H), 2.50-2.13 (m, 18H), 2.12-1.99 (m, 3H), 1.91-1.82 (m, 3H), 1.77-1.68 (m, 1H), 1.67-1.55 (m, 2H), 1.49-1.42 (m, 1H), 0.97 (s, 3H). LC/MS (ESI) m/z 1482.6; [M+H]⁺ calcd for C₇₄H₉₂ClF₃N₁₁O₁₀S₃⁺: 1482.58.

### Example 6: Preparation of Compound #7

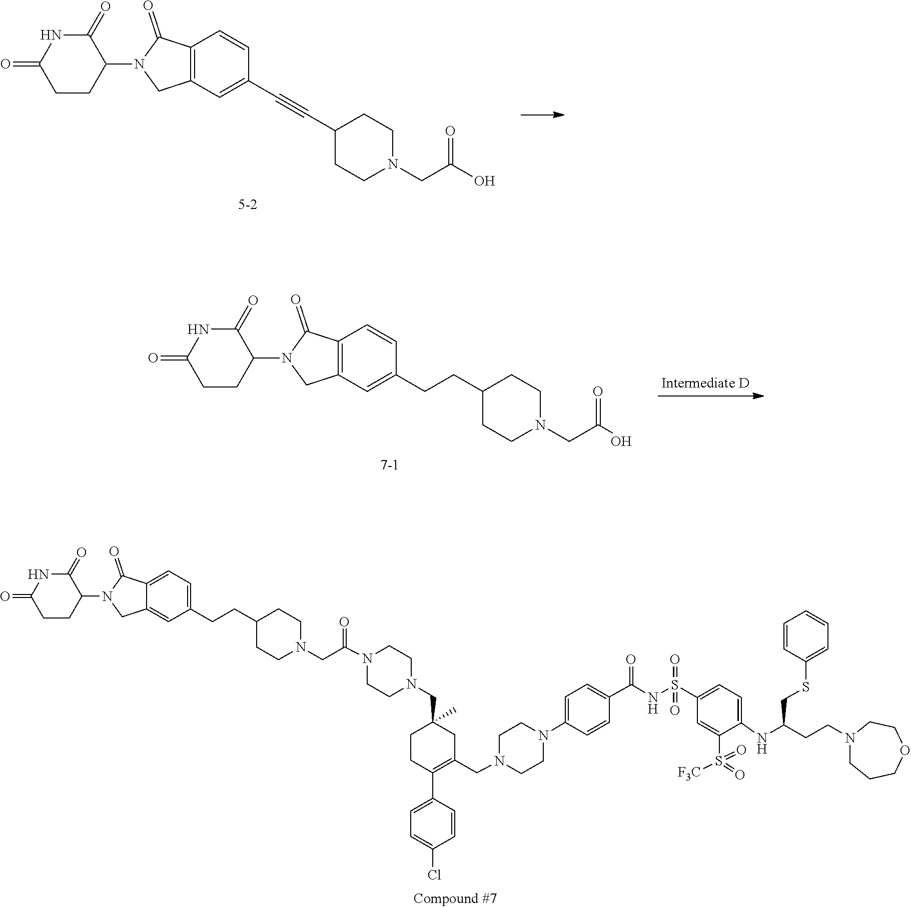 [SMILES: O=C(O)CN1CCC(C#Cc2ccc3c(c2)CN(C2CCC(=O)NC2=O)C3=O)CC1 ; O=C(O)CN1CCC(CCc2ccc3c(c2)CN(C2CCC(=O)NC2=O)C3=O)CC1 ; C[C@@]1(CN2CCN(C(=O)CN3CCC(CCc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #7 was Prepared by Following General Procedure D:

2-(4-(2-(2-(2,6-dioxopiperidin-3-yl)-1-oxoisoindolin-5-yl)ethyl) piperidin-1-yl) acetic acid (7-1) (18 mg, 85% yield). LC/MS (ESI) m/z 414.2; [M+H]⁺ calcd for C₂₂H₂₈N₃O₅⁺: 414.20.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-(2-(2-(2,6-dioxopiperidin-3-yl)-1-oxoisoindolin-5-yl)ethyl) piperidin-1-yl) acetyl) piperazin-1-yl)methyl)-4-methyl-3,4,5,6-tetrahydro-[1,1′-biphenyl]-2-yl)methyl) piperazin-1-yl)benzamide (compound #7) (7.24 mg, 36% yield). ¹H NMR (600 MHZ, Chloroform-d) δ 8.54 (s, 1H), 8.34 (t, J=2.1 Hz, 1H), 8.05 (d, J=9.1 Hz, 1H), 7.82 (dd, J=8.9, 3.4 Hz, 2H), 7.80-7.75 (m, 1H), 7.40-7.35 (m, 2H), 7.34-7.21 (m, 7H), 7.05-6.98 (m, 3H), 6.77 (d, J=8.6 Hz, 2H), 6.56 (d, J =9.4 Hz, 1H), 5.30-5.23 (m, 1H), 4.46 (dd, J=16.2, 2.5 Hz, 1H), 4.33 (d, J=16.0 Hz, 1H), 3.88 (d, J=10.5 Hz, 1H), 3.77 (t, J=6.1 Hz, 2H), 3.75-3.71 (m, 2H), 3.63-3.47 (m, 4H), 3.37 (s, 2H), 3.26 (t, J=5.3 Hz, 4H), 3.10 (dd, J=13.9, 5.0 Hz, 1H), 3.07-2.99 (m, 3H), 2.95-2.84 (m, 4H), 2.83-2.61 (m, 9H), 2.54 (d, J=17.6 Hz, 4H), 2.45-2.32 (m, 6H), 2.31-2.15 (m, 6H), 2.14-2.06 (m, 1H), 1.98-1.87 (m, 4H), 1.82-1.70 (m, 3H), 1.69-1.53 (m, 4H), 1.50-1.43 (m, OH), 1.43-1.33 (m, 2H), 0.96 (s, 3H). LC/MS (ESI) m/z 1467.7; [M+H]⁺ calcd for C₇₄H₉₁ClF₃N₁₀O₁₀S₃⁺: 1467.57.

### Example 7: Preparation of Compounds #8-9

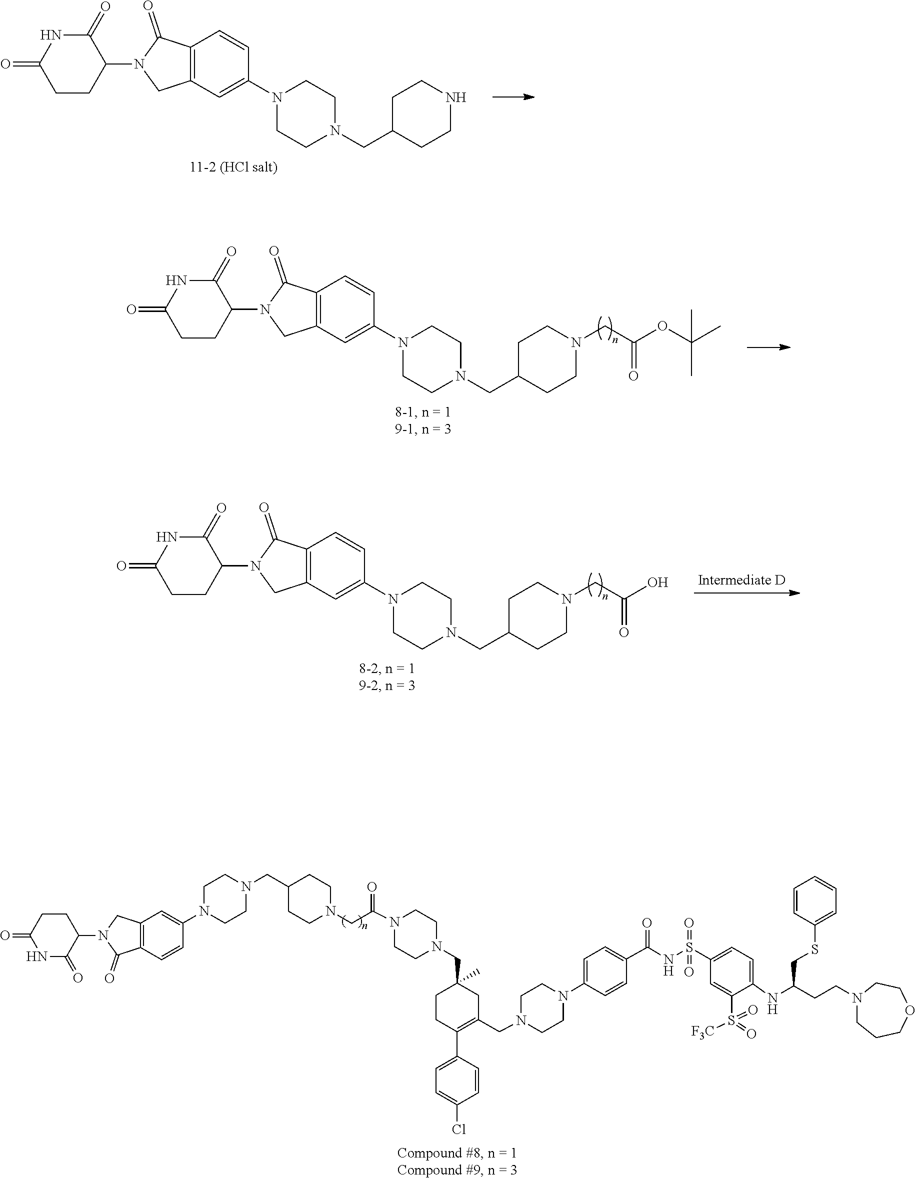

Compounds #8-9 were Prepared by Following General Procedure A:

tert-butyl 2-(4-((4-(2-(2,6-dioxopiperidin-3-yl)-1-oxoisoindolin-5-yl) piperazin-1-yl)methyl) piperidin-1-yl)acetate (8-1) (21 mg, 29% yield). ¹H NMR (600 MHZ, Chloroform-d) δ 8.12 (s, 1H), 7.75 (d, J=8.6 Hz, 1H), 7.00 (dd, J=8.7, 2.3 Hz, 1H), 6.89 (d, J=2.2 Hz, 1H), 5.21 (dd, J=13.4, 5.0 Hz, 1H), 4.42 (d, J=15.5 Hz, 1H), 4.27 (d, J=15.5 Hz, 1H), 3.33 (t, J=4.9 Hz, 4H), 3.14 (s, 2H), 3.02-2.95 (m, 2H), 2.95-2.77 (m, 2H), 2.57 (t, J=5.0 Hz, 4H), 2.39-2.29 (m, 1H), 2.26 (d, J=7.1 Hz, 2H), 2.24-2.15 (m, 3H), 1.82-1.74 (m, 2H), 1.58-1.50 (m, 1H), 1.49 (s, 9H), 1.41-1.31 (m, 2H). LC/MS (ESI) m/z 540.3; [M+H]⁺ calcd for C₂₉H₄₂N₅O₅⁺: 540.32.

tert-butyl 4-(4-((4-(2-(2,6-dioxopiperidin-3-yl)-1-oxoisoindolin-5-yl) piperazin-1-yl)methyl) piperidin-1-yl) butanoate (9-1) (12 mg, 35% yield). ¹H NMR (600 MHZ, Chloroform-d) δ 8.63 (s, 1H), 7.74 (dd, J=8.5, 1.7 Hz, 1H), 7.00 (d, J=8.3 Hz, 1H), 6.89 (d, J=2.3 Hz, 1H), 5.20 (dd, J=13.0, 5.2 Hz, 1H), 4.41 (d, J=15.4 Hz, 1H), 4.26 (d, J=15.5 Hz, 1H), 3.34 (t, J=5.0 Hz, 4H), 3.11 (s, 2H), 2.95 (d, J=10.7 Hz, 2H), 2.92-2.77 (m, 2H), 2.61 (t, J=5.0 Hz, 4H), 2.47-2.42 (m, 2H), 2.39-2.24 (m, 1H), 2.19-2.12 (m, 3H), 1.73-1.65 (m, 2H), 1.53-1.45 (m, 10H), 1.44-1.35 (m, 3H), 1.35-1.23 (m, 3H). LC/MS (ESI) m/z 568.4; [M+H]⁺ calcd for C₃₁H₄₆N₅O₅⁺: 568.35.

2-(4-((4-(2-(2,6-dioxopiperidin-3-yl)-1-oxoisoindolin-5-yl) piperazin-1-yl)methyl) piperidin-1-yl) acetic acid (8-2) (12 mg, quantitative yield). LC/MS (ESI) m/z 484.2; [M+H]⁺ calcd for C₂₅H₃₄N₅O₅⁺: 484.26.

4-(4-((4-(2-(2,6-dioxopiperidin-3-yl)-1-oxoisoindolin-5-yl) piperazin-1-yl)methyl) piperidin-1-yl) butanoic acid (9-2) (7 mg, quantitative yield). LC/MS (ESI) m/z 512.3; [M+H]⁺ calcd for C₂₇H₃₈N₅O₅⁺: 512.29.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-((4-(2-(2,6-dioxopiperidin-3-yl)-1-oxoisoindolin-5-yl) piperazin-1-yl)methyl) piperidin-1-yl) acetyl) piperazin-1-yl)methyl)-4-methyl-3,4,5,6-tetrahydro-[1,1′-biphenyl]-2-yl)methyl) piperazin-1-yl)benzamide (compound #8) (5.38 mg, 34% yield1H NMR (600 MHZ, Chloroform-d) δ 8.31 (t, J=2.4 Hz, 1H), 8.04 (d, J=9.0 Hz, 1H), 7.78 (dd, J=8.9, 2.1 Hz, 2H), 7.72 (dd, J=9.5, 2.6 Hz, 1H), 7.38-7.33 (m, 2H), 7.32-7.20 (m, 6H), 6.98 (dq, J=8.9, 2.2 Hz, 3H), 6.88 (s, 1H), 6.80-6.75 (m, 2H), 6.56 (d, J=9.3 Hz, 1H), 5.15 (dd, J=13.5, 5.1 Hz, 1H), 4.40 (d, J=15.8 Hz, 1H), 4.26 (d, J=15.8 Hz, 1H), 3.86 (s, 1H), 3.75 (t, J=6.1 Hz, 2H), 3.73-3.64 (m, 2H), 3.60-3.44 (m, 4H), 3.45-3.29 (m, 6H), 3.25 (t, J=5.1 Hz, 4H), 3.09 (dd, J=14.0, 4.9 Hz, 1H), 3.06-2.98 (m, 3H), 2.92-2.81 (m, 4H), 2.79-2.47 (m, 14H), 2.44-2.14 (m, 14H), 2.12-2.02 (m, 1H), 1.93-1.85 (m, 3H), 1.82 (d, J=13.0 Hz, 2H), 1.76-1.66 (m, 1H), 1.66-1.54 (m, 3H), 1.48-1.39 (m, 1H), 1.38-1.28 (m, 2H), 0.94 (s, 3H). LC/MS (ESI) m/z 1537.5; [M+H]⁺ calcd for C₇₇H₉₇ClF₃N₁₂O₁₀S₃⁺: 1537.62.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(4-(4-((4-(2-(2,6-dioxopiperidin-3-yl)-1-oxoisoindolin-5-yl) piperazin-1-yl)methyl) piperidin-1-yl) butanoyl) piperazin-1-yl)methyl)-4-methyl-3,4,5,6-tetrahydro-[1,1′-biphenyl]-2-yl)methyl) piperazin-1-yl)benzamide (compound #9) (0.95 mg, 7% yield). ¹H NMR (600 MHZ, Chloroform-d) δ 8.38-8.35 (m, 1H), 8.00 (d, J=8.4 Hz, 2H), 7.95 (d, J=8.4 Hz, 1H), 7.72 (d, J=8.5 Hz, 1H), 7.39-7.34 (m, 2H), 7.32-7.25 (m, 5H), 7.25-7.20 (m, 1H), 7.02-6.96 (m, 3H), 6.88 (s, 1H), 6.76 (d, J=8.7 Hz, 2H), 6.50 (d, J=9.2 Hz, 1H), 5.16 (dd, J=13.4, 5.1 Hz, 1H), 4.40 (d, J=15.8 Hz, 1H), 4.26 (d, J=15.8 Hz, 1H), 3.81 (s, 1H), 3.77-3.72 (m, 2H), 3.71-3.62 (m, 4H), 3.43-3.39 (m, 4H), 3.30 (t, J=5.1 Hz, 4H), 3.17 (s, 2H), 3.12-3.01 (m, 4H), 2.96 (dd, J=13.8, 7.6 Hz, 1H), 2.90-2.78 (m, 3H), 2.71-2.49 (m, 10H), 2.45 (d, J=13.8 Hz, 1H), 2.42-2.26 (m, 10H), 2.24-2.05 (m, 12H), 1.97 (d, J=13.9 Hz, 1H), 1.90-1.73 (m, 5H), 1.72-1.53 (m, 5H), 1.50-1.44 (m, 1H), 1.36-1.29 (m, 2H), 0.97 (s, 3H). LC/MS (ESI) m/z 1565.8; [M+H]⁺ calcd for C₇₉H₁₀₁ClF₃N₁₂O₁₀S₃⁺: 1565.66.

### Example 8: Preparation of Compound #10

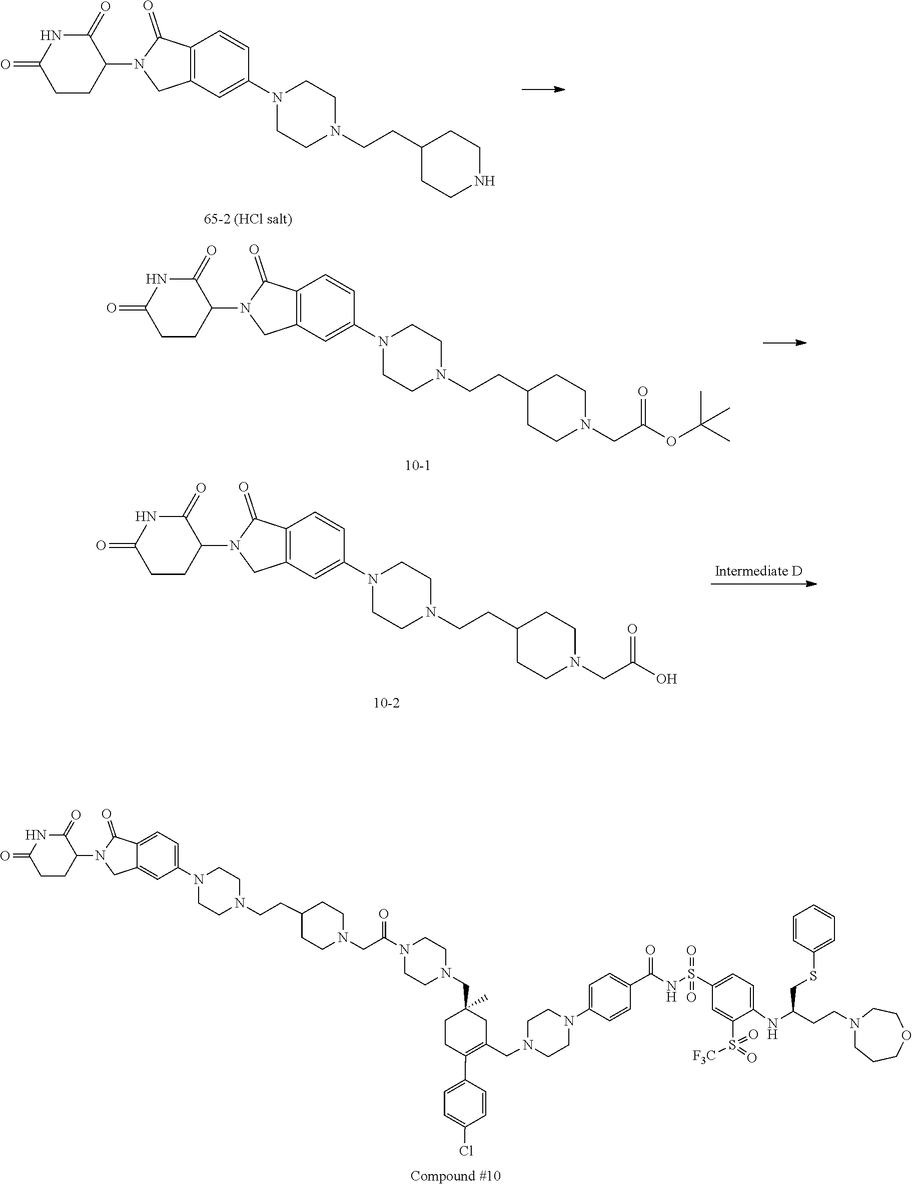 [SMILES: O=C1CCC(N2Cc3cc(N4CCN(CCC5CCNCC5)CC4)ccc3C2=O)C(=O)N1 ; CC(C)(C)OC(=O)CN1CCC(CCN2CCN(c3ccc4c(c3)CN(C3CCC(=O)NC3=O)C4=O)CC2)CC1 ; O=C(O)CN1CCC(CCN2CCN(c3ccc4c(c3)CN(C3CCC(=O)NC3=O)C4=O)CC2)CC1 ; C[C@@]1(CN2CCN(C(=O)CN3CCC(CCN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #10 was Prepared by Following General Procedure A:

tert-butyl 2-(4-(2-(4-(2-(2,6-dioxopiperidin-3-yl)-1-oxoisoindolin-5-yl) piperazin-1-yl)ethyl) piperidin-1-yl)acetate (10-1) (22 mg, 40% yield). ¹H NMR (600 MHZ, Chloroform-d) 8.63 (s, 1H), 7.74 (dd, J=9.0, 3.6 Hz, 1H), 7.00 (d, J=8.4 Hz, 1H), 6.89 (s, 1H), 5.20 (dd, J=13.4, 5.1 Hz, 1H), 4.42 (dd, J=15.3, 4.3 Hz, 1H), 4.26 (dd, J=15.7, 4.0 Hz, 1H), 3.34 (t, J=5.0 Hz, 4H), 3.11 (s, 2H), 2.95 (d, J=11.0 Hz, 2H), 2.92-2.77 (m, 2H), 2.61 (t, J=5.0 Hz, 4H), 2.47-2.42 (m, 2H), 2.38-2.25 (m, 1H), 2.19-2.12 (m, 3H), 1.69 (d, 2H), 1.53-1.45 (m, 10H), 1.43-1.35 (m, 2H), 1.35-1.24 (m, 2H). LC/MS (ESI) m/z 554.4; [M+H]⁺ calcd for C₃₀H₄₄N₅O₅⁺: 554.33.

2-(4-(2-(4-(2-(2,6-dioxopiperidin-3-yl)-1-oxoisoindolin-5-yl) piperazin-1-yl)ethyl) piperidin-1-2 yl) acetic acid (10-2) (12 mg, quantitative yield). LC/MS (ESI) m/z 498.3; [M+H]⁺ calcd for C₂₆H₃₆N₅O₅⁺: 498.27.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-(2-(4-(2-(2,6-dioxopiperidin-3-yl)-1-oxoisoindolin-5-yl) piperazin-1-yl)ethyl) piperidin-1-yl) acetyl) piperazin-1-yl)methyl)-4-methyl-3,4,5,6-tetrahydro-[1,1′-biphenyl]-2-yl)methyl) piperazin-1-yl)benzamide (compound #10) (1.18 mg, 6% yield). ¹H NMR (600 MHz, Chloroform-d) δ 8.32 (d, J=2.2 Hz, 1H), 8.04 (dd, J=9.2, 2.2 Hz, 1H), 7.79 (d, J=8.8 Hz, 2H), 7.74 (d, J=8.6 Hz, 1H), 7.39-7.34 (m, 2H), 7.32-7.20 (m, 6H), 7.03-6.97 (m, 3H), 6.92 (s, 1H), 6.79 (d, J=8.9 Hz, 2H), 6.57 (d, J=9.3 Hz, 1H), 5.17 (dd, J=13.4, 5.0 Hz, 1H), 4.41 (d, J=15.8 Hz, 1H), 4.28 (d, J=15.9 Hz, 1H), 3.86 (s, 1H), 3.75 (t, J=6.1 Hz, 2H), 3.71-3.56 (m, 3H), 3.54-3.39 (m, 10H), 3.30-3.16 (m, 4H), 3.11-3.05 (m, 1H), 3.02 (dd, J=13.9, 7.2 Hz, 1H), 2.96-2.74 (m, 10H), 2.74-2.45 (m, 12H), 2.45-2.15 (m, 10H), 2.09-1.74 (m, 5H), 1.69 (d, J=11.5 Hz, 1H), 1.66-1.56 (m, 6H), 1.54-1.46 (m, 1H), 1.46-1.39 (m, 1H), 1.38-1.21 (m, 2H), 0.95 (s, 3H). LC/MS (ESI) m/z 1551.7; [M+H]⁺ calcd for C₇₈H₉₉ClF₃N₁₂O₁₀S₃⁺: 1551.64.

### Example 9: Preparation of Compound #12

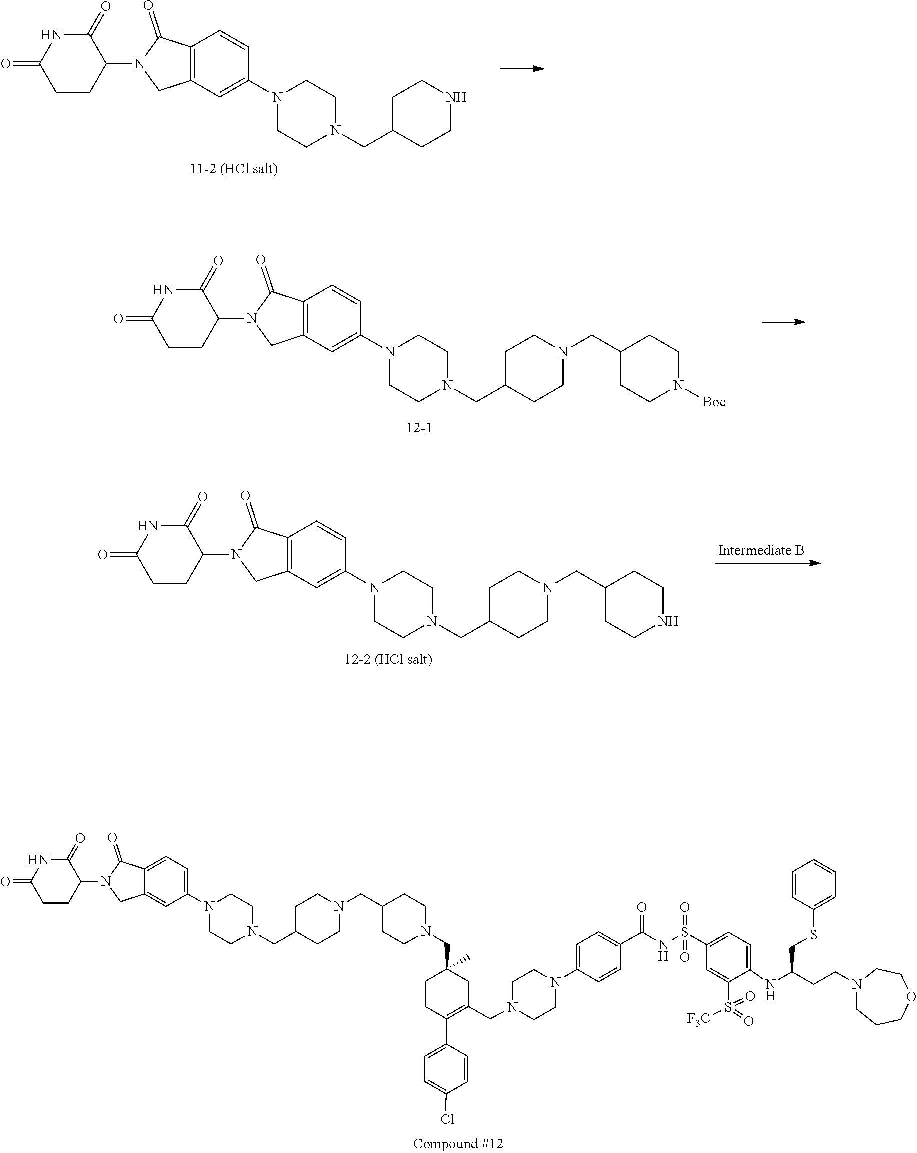 [SMILES: O=C1CCC(N2Cc3cc(N4CCN(CC5CCNCC5)CC4)ccc3C2=O)C(=O)N1 ; CC(C)(C)OC(=O)N1CCC(CN2CCC(CN3CCN(c4ccc5c(c4)CN(C4CCC(=O)NC4=O)C5=O)CC3)CC2)CC1 ; O=C1CCC(N2Cc3cc(N4CCN(CC5CCN(CC6CCNCC6)CC5)CC4)ccc3C2=O)C(=O)N1 ; C[C@@]1(CN2CCC(CN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #12 was Prepared by Following General Procedure C:

tert-butyl 4-((4-((4-(2-(2,6-dioxopiperidin-3-yl)-1-oxoisoindolin-5-yl) piperazin-1-yl)methyl) piperidin-1-yl)methyl) piperidine-1-carboxylate (12-1) (17 mg, 29% yield). ¹H NMR (600 MHZ, Chloroform-d) δ 8.06 (s, 1H), 7.75 (d, J=8.5 Hz, 1H), 7.01 (dd, J=8.6, 2.1 Hz, 1H), 6.89 (d, J=2.1 Hz, 1H), 5.22 (dd, J=13.3, 5.1 Hz, 1H), 4.43 (d, J=15.5 Hz, 1H), 4.27 (d, J=15.6 Hz, 1H), 4.21-3.95 (m, 3H), 3.33 (t, J=5.0 Hz, 4H), 3.00-2.80 (m, 4H), 2.78-2.64 (m, 2H), 2.58 (t, J=5.1 Hz, 4H), 2.40-2.30 (m, 1H), 2.29-2.13 (m, 3H), 2.03-1.62 (m, 9H), 1.61-1.51 (m, 1H), 1.47 (s, 9H), 1.19-1.03 (m, 3H). LC/MS (ESI) m/z 623.5; [M+H]⁺ calcd for C₃₄H₅₁N₆O₅⁺: 623.39.

3-(1-oxo-5-(4-((1-(piperidin-4-ylmethyl) piperidin-4-yl)methyl) piperazin-1-yl) isoindolin-2-yl) piperidine-2,6-dione hydrochloride (12-2) (9 mg, quantitative yield). LC/MS (ESI) m/z 523.3; [M+H]⁺ calcd for C₂₉H₄₃N₆O₃⁺: 523.34.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-((4-(2-(2,6-dioxopiperidin-3-yl)-1-oxoisoindolin-5-yl) piperazin-1-yl)methyl) piperidin-1-yl)methyl) piperidin-1-yl)methyl)-4-methyl-3,4,5,6-tetrahydro-[1,1′-biphenyl]-2-yl)methyl) piperazin-1-yl)benzamide (compound #12) (0.73 mg, 3% yield). ¹H NMR (600 MHz, Chloroform-d) δ 8.32 (s, 1H), 8.02 (d, J=9.2 Hz, 1H), 7.83 (d, J=8.5 Hz, 2H), 7.72 (dd, J=8.4, 1.7 Hz, 2H), 7.37 (d, J=7.6 Hz, 2H), 7.32-7.19 (m, 5H), 7.02-6.95 (m, 3H), 6.87 (s, 1H), 6.77 (d, J=8.7 Hz, 2H), 6.54 (d, J=9.2 Hz, 1H), 5.20-5.10 (m, 1H), 4.40 (d, J=15.7 Hz, 1H), 4.26 (d, J=15.8 Hz, 1H), 3.88-3.77 (m, 3H), 3.75 (dt, J=6.9, 3.5 Hz, 2H), 3.69-3.62 (m, 3H), 3.40 (t, J=1.7 Hz, 4H), 3.31 (s, 4H), 3.29-3.15 (m, 3H), 3.12-2.96 (m, 3H), 2.96-2.73 (m, 9H), 2.71-2.44 (m, 15H), 2.40-2.03 (m, 10H), 1.91-1.80 (m, 3H), 1.80-1.72 (m, 3H), 1.71-1.46 (m, 7H), 0.90 (s, 3H). LC/MS (ESI) m/z 1508.7; [M+H]⁺ calcd for C₇₇H₉₈ClF₃N₁₁O₉S₃⁺: 1508.63.

### Example 10: Preparation of Compound #13

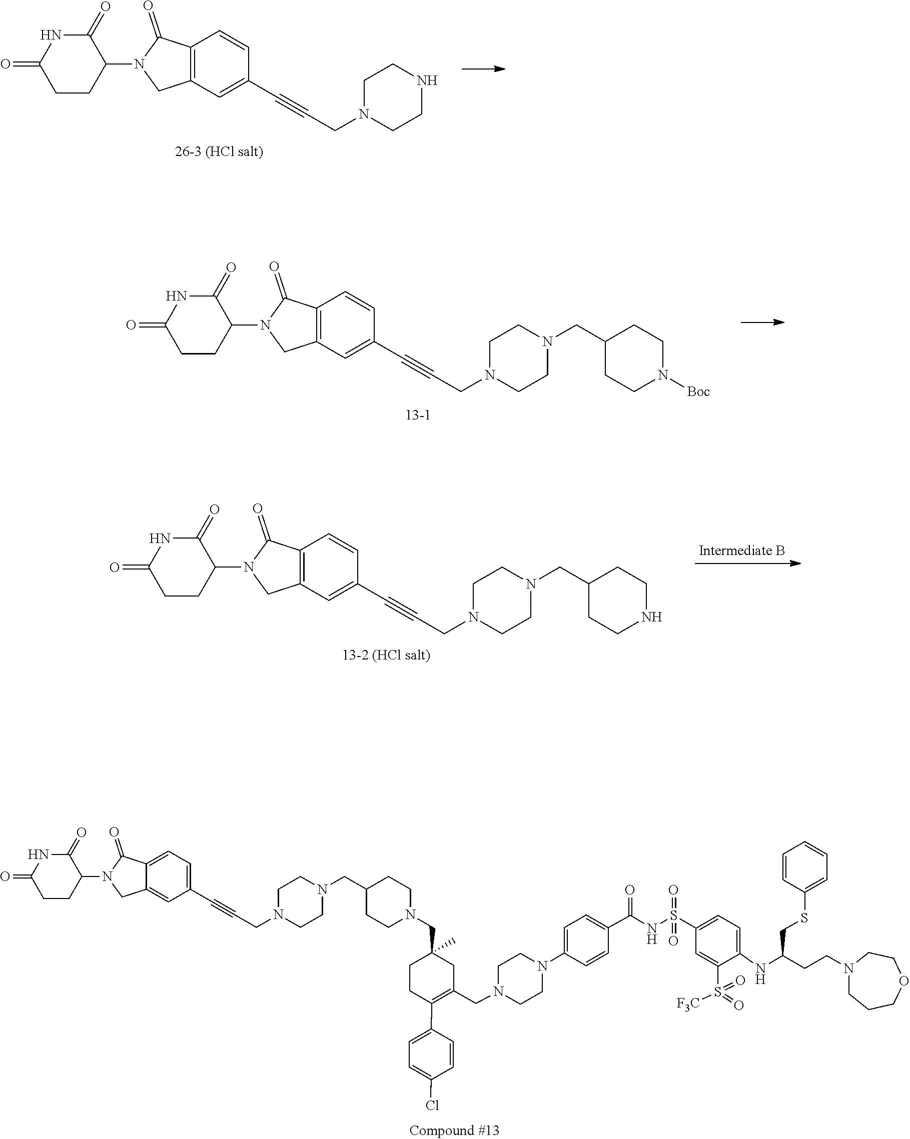 [SMILES: O=C1CCC(N2Cc3cc(C#CCN4CCNCC4)ccc3C2=O)C(=O)N1 ; CC(C)(C)OC(=O)N1CCC(CN2CCN(CC#Cc3ccc4c(c3)CN(C3CCC(=O)NC3=O)C4=O)CC2)CC1 ; O=C1CCC(N2Cc3cc(C#CCN4CCN(CC5CCNCC5)CC4)ccc3C2=O)C(=O)N1 ; C[C@@]1(CN2CCC(CN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Compound #13 was prepared by following General Procedure C:

tert-butyl 4-((4-(3-(2-(2,6-dioxopiperidin-3-yl)-1-oxoisoindolin-5-yl) prop-2-yn-1-yl) piperazin-1-yl)methyl) piperidine-1-carboxylate (13-1) (65 mg, 72% yield). ¹H NMR (600 MHz, Chloroform-d) δ 8.20 (s, 1H), 7.84 (d, 1H), 7.57 (dd, J=7.9, 1.3 Hz, 1H), 7.55 (s, 1H), 5.23 (dd, J=13.4, 5.1 Hz, 1H), 4.50 (d, J=16.0 Hz, 1H), 4.34 (d, J=16.0 Hz, 1H), 4.21-3.98 (m, 3H), 3.56 (s, 2H), 2.95 (ddd, J=17.8, 4.8, 2.5 Hz, 1H), 2.86 (ddd, J=18.1, 13.4, 5.4 Hz, 1H), 2.79-2.61 (m, 5H), 2.59-2.45 (m, 3H), 2.38 (qd, J=13.2, 4.7 Hz, 1H), 2.29-2.20 (m, 3H), 1.86-1.71 (m, 3H), 1.70-1.61 (m, 1H), 1.47 (s, 9H), 1.09 (qd, J=12.3, 4.2 Hz, 2H). LC/MS (ESI) m/z 564.3; [M+H]⁺ calcd for C₃₁H₄₂N₅O₅⁺: 564.32.

3-(1-oxo-5-(3-(4-(piperidin-4-ylmethyl) piperazin-1-yl) prop-1-yn-1-yl) isoindolin-2-yl) piperidine-2,6-dione hydrochloride (13-2) (32 mg, quantitative yield). LC/MS (ESI) m/z 464.3; [M+H]⁺ calcd for C₂₆H₃₄N₅O₃⁺: 464.27.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-(3-(2-(2,6-12 dioxopiperidin-3-yl)-1-oxoisoindolin-5-yl) prop-2-yn-1-yl) piperazin-1-yl)methyl) piperidin-1-yl)methyl)-4-methyl-3,4,5,6-tetrahydro-[1,1′-biphenyl]-2-yl)methyl) piperazin-1-14 yl)benzamide (compound #13) (4.34 mg, 20% yield). ¹H NMR (600 MHz, Chloroform-d) & 8.30 (dd, J=5.8, 2.2 Hz, 1H), 8.02-7.97 (m, 1H), 7.84 (dd, J=8.9, 3.3 Hz, 2H), 7.77 (dd, J=16.7, 8.0 Hz, 1H), 7.55-7.47 (m, 2H), 7.40-7.35 (m, 2H), 7.33-7.19 (m, 6H), 6.98 (dd, J=8.3, 1.6 Hz, 2H), 6.75 (dd, J=11.7, 8.7 Hz, 2H), 6.48 (d, J=9.3 Hz, 1H), 5.19 (dd, J=13.4, 5.1 Hz, 1H), 4.43 (d, J=16.3 Hz, 1H), 4.29 (d, J=16.3 Hz, 1H), 3.81 (s, 1H), 3.77 (td, J=6.1, 1.3 Hz, 2H), 3.74-3.67 (m, 2H), 3.58 (d, J=6.9 Hz, 2H), 3.26-3.14 (m, 4H), 3.11 (dd, J=13.8, 4.2 Hz, 1H), 3.06-2.95 (m, 3H), 2.92-2.46 (m, 16H), 2.44-2.15 (m, 16H), 2.13-2.04 (m, 1H), 2.04-1.84 (m, 3H), 1.81-1.49 (m, 8H), 1.48-1.35 (m, 1H), 0.98 (d, J=9.1 Hz, 3H). LC/MS (ESI) m/z 1449.5; [M+H]⁺ calcd for C₇₄H₈₉ClF₃N₁₀O₉S₃⁺: 1449.56.

### Example 11: Preparation of Compounds #14-15

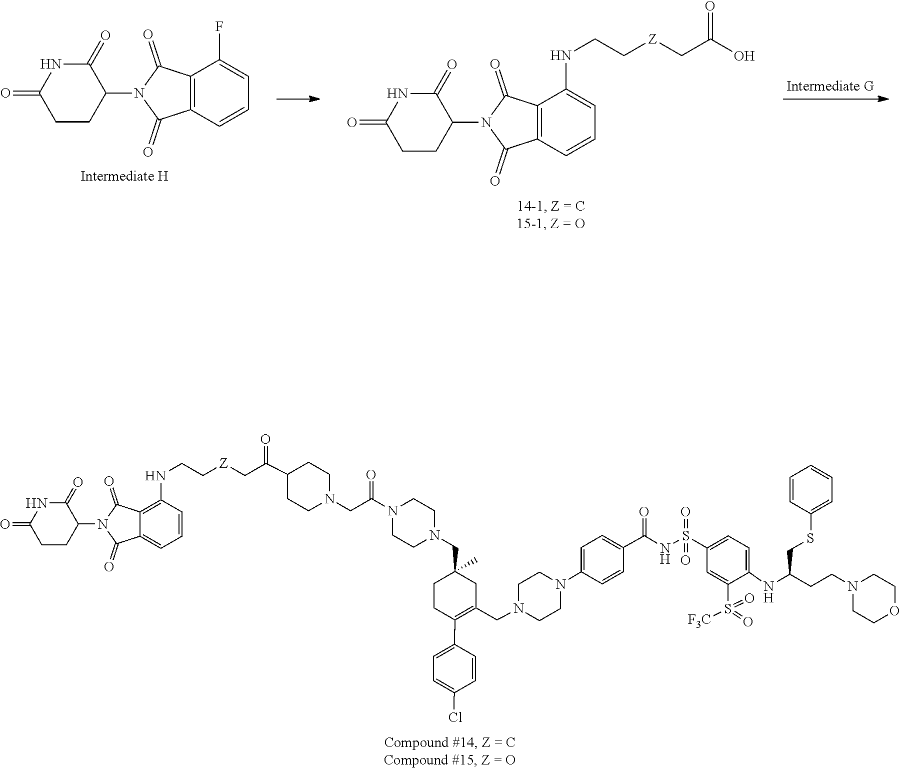

Compounds #14-15 were Prepared by Following General Procedure F:

5-((2-(2,6-dioxopiperidin-3-yl)-1,3-dioxoisoindolin-4-yl)amino) pentanoic acid (14-1) (175 mg, 65% yield). ¹H NMR (599 MHz, DMSO-d₆) δ 11.08 (s, 1H), 7.58 (dd, J=8.5, 7.1 Hz, 1H), 7.10 (d, J=8.6 Hz, 1H), 7.02 (d, J=7.0 Hz, 1H), 6.57 (t, J=6.0 Hz, 1H), 5.04 (dd, J=12.9, 5.5 Hz, 1H), 3.44-3.21 (m, 2H), 2.94-2.81 (m, 1H), 2.64-2.46 (m, 2H), 2.29-2.24 (m, 2H), 2.06-1.99 (m, 1H), 1.61-1.53 (m, 4H). LC/MS (ESI) m/z 374.1; [M+H]⁺ calcd for C₁₈H₂₀N₃O₆⁺: 374.13.

2-(2-((2-(2,6-dioxopiperidin-3-yl)-1,3-dioxoisoindolin-4-yl)amino) ethoxy) acetic acid (15-1) (143 mg, 53% yield). ¹H NMR (599 MHz, DMSO-d₆) δ 11.09 (s, 1H), 7.58 (dd, J=8.6, 7.1 Hz, 1H), 7.15 (d, J=8.6 Hz, 1H), 7.04 (d, J=7.0 Hz, 1H), 6.68 (s, 1H), 5.05 (dd, J=12.9, 5.4 Hz, 1H), 4.03 (s, 2H), 3.68 (q, J=5.5 Hz, 2H), 3.53-3.46 (m, 2H), 2.94-2.82 (m, 1H), 2.65-2.45 (m, 2H), 2.07-1.97 (m, 1H). LC/MS (ESI) m/z 376.2; [M+H]⁺ calcd for C₁₇H₁₈N₃O₇+: 376.11.

4-(4-((4′-chloro-4-((4-(2-(4-(5-((2-(2,6-dioxopiperidin-3-yl)-1,3-dioxoisoindolin-4-yl)amino) pentanoyl) piperazin-1-yl) acetyl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-((trifluoromethyl) sulfonyl)phenyl) sulfonyl)benzamide (compound #14) (2.99 mg, 25% yield). ¹H NMR (600 MHz, Chloroform-d) δ 9.18 (s, 1H), 8.31 (d, J=2.2 Hz, 1H), 8.06 (d, J=9.1 Hz, 1H), 7.71 (d, J=8.4 Hz, 2H), 7.48 (dd, J=8.5, 7.1 Hz, 1H), 7.36 (d, J=7.2 Hz, 2H), 7.32-7.20 (m, 6H), 7.07 (d, J=7.1 Hz, 1H), 7.02-6.94 (m, 3H), 6.88 (d, J=8.6 Hz, 1H), 6.72 (s, 2H), 6.59 (s, 1H), 6.23 (s, 1H), 4.88 (dd, J=12.0, 5.5 Hz, 1H), 3.87 (s, 1H), 3.69-3.45 (m, 8H), 3.45-3.35 (m, 1H), 3.34-3.12 (m, 10H), 3.09 (dd, J=13.9, 5.0 Hz, 1H), 3.01 (dd, J=13.8, 7.1 Hz, 1H), 2.92-2.79 (m, 2H), 2.79-2.65 (m, 3H), 2.63-2.14 (m, 22H), 2.13-2.06 (m, 3H), 1.98-1.85 (m, 1H), 1.81-1.50 (m, 8H), 1.49-1.40 (m, 1H), 0.95 (s, 3H). LC/MS (ESI) m/z 1539.7; [M+H]⁺ calcd for C₇₅H₉₁ClF₃N₁₂O₁₂S₃⁺: 1539.57.

4-(4-((4′-chloro-4-((4-(2-(4-(2-(2-((2-(2,6-dioxopiperidin-3-yl)-1,3-dioxoisoindolin-4-yl)amino) ethoxy) acetyl) piperazin-1-yl) acetyl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-30 (phenylthio) butan-2-yl)amino)-3-((trifluoromethyl) sulfonyl)phenyl) sulfonyl)benzamide 32 (compound #15) (2.1 mg, 18% yield). ¹H NMR (600 MHZ, Chloroform-d) δ 9.52 (s, 1H), 8.32 (s, 1H), 8.12-8.03 (m, 1H), 7.71 (d, J=8.4 Hz, 2H), 7.49 (t, J=7.8 Hz, 1H), 7.37 (d, J=7.6 Hz, 2H), 7.34-7.20 (m, 6H), 7.10 (d, J=7.1 Hz, 1H), 6.99 (d, J=7.9 Hz, 3H), 6.89 (d, J=8.6 Hz, 1H), 6.72 (s, 2H), 6.59 (s, 1H), 6.52 (s, 1H), 4.94-4.82 (m, 1H), 4.26-4.07 (m, 2H), 3.94-3.81 (m, 1H), 3.80-3.33 (m, 16H), 3.30-3.06 (m, 7H), 3.06-2.76 (m, 3H), 2.76-2.63 (m, 2H), 2.63-2.15 (m, 22H), 2.16-2.06 (m, 2H), 1.97-1.85 (m, 1H), 1.75-1.48 (m, 4H), 1.48-1.37 (m, 1H), 0.94 (s, 3H). LC/MS (ESI) m/z 1541.7; [M+H]⁺ calcd for C₇₄H₈₉ClF₃N₁₂O₁₃S₃⁺: 1541.55.

### Example 12: Preparation of Compound #16

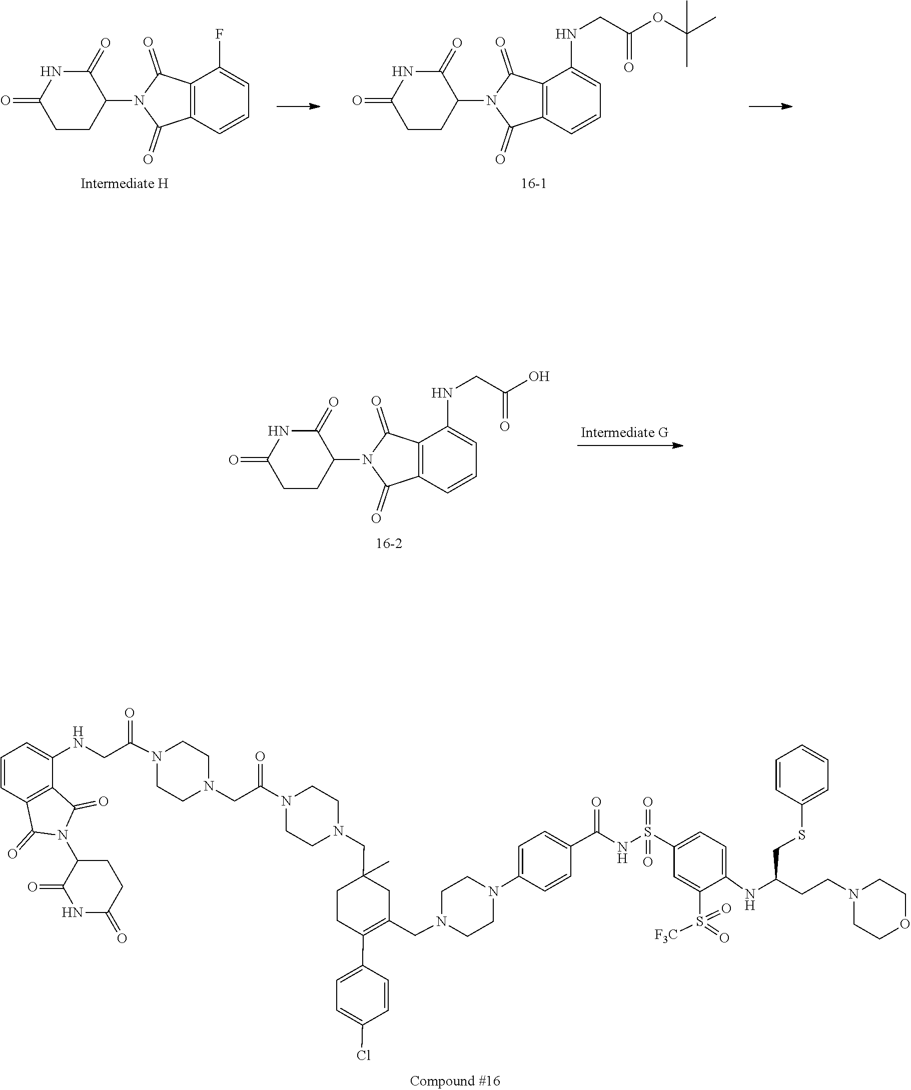 [SMILES: O=C1CCC(N2C(=O)c3cccc(F)c3C2=O)C(=O)N1 ; CC(C)(C)OC(=O)CNc1cccc2c1C(=O)N(C1CCC(=O)NC1=O)C2=O ; O=C(O)CNc1cccc2c1C(=O)N(C1CCC(=O)NC1=O)C2=O ; CC1(CN2CCN(C(=O)CN3CCN(C(=O)CNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

### Compound #16 was Prepared by Following General Procedure G:

tert-butyl(2-(2,6-dioxopiperidin-3-yl)-1,3-dioxoisoindolin-4-yl)glycinate (16-1) (405 mg, 58% yield). ¹H NMR (600 MHZ, Chloroform-d) δ 7.99 (s, 1H), 7.51 (dd, J=8.4, 7.2 Hz, 1H), 7.16 (d, J=7.2 Hz, 1H), 6.76 (d, J=8.4 Hz, 1H), 6.71 (t, J=5.5 Hz, 1H), 4.93 (dd, J=12.5, 5.4 Hz, 1H), 3.95 (d, J=5.5 Hz, 2H), 2.92-2.87 (m, 1H), 2.88-2.68 (m, 2H), 2.16-2.09 (m, 1H), 1.50 (s, 9H). LC/MS (ESI) m/z 388.2; [M+H]⁺ calcd for C₁₉H₂₂N₃O₆⁺: 388.15.

(2-(2,6-dioxopiperidin-3-yl)-1,3-dioxoisoindolin-4-yl)glycine (16-2) (4.2 mg, quantitative yield). LC/MS (ESI) m/z 332.1; [M+H]⁺ calcd for C₁₅H₁₄N₃O₆⁺: 332.09.

4-(4-((4′-chloro-4-((4-(2-(4-((2-(2,6-dioxopiperidin-3-yl)-1,3-dioxoisoindolin-4-yl) glycyl) piperazin-1-yl) acetyl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-((trifluoromethyl) sulfonyl)phenyl) sulfonyl)benzamide (compound #16) (3.56 mg, 32% yield). ¹H NMR (600 MHZ, Chloroform-d) δ 8.30 (d, 1H), 8.04 (d, 1H), 7.71 (d, J=8.4 Hz, 2H), 7.45 (t, J=24.5 Hz, 1H), 7.36 (d, J=7.6 Hz, 2H), 7.32-7.21 (m, 6H), 7.11 (d, J=7.2 Hz, 1H), 7.04-6.90 (m, 2H), 6.81-6.64 (m, 3H), 6.60 (s, 1H), 4.89 (dd, 1H), 4.06-3.81 (m, 3H), 3.77-3.35 (m, 13H), 3.32-3.13 (m, 6H), 3.09 (dd, J=13.9, 5.0 Hz, 1H), 3.01 (dd, J=13.9, 7.1 Hz, 1H), 2.95-2.64 (m, 5H), 2.64-2.15 (m, 20H), 2.13-2.02 (m, 2H), 2.02-1.86 (m, 1H), 1.77-1.38 (m, 5H), 0.96 (s, 3H). LC/MS (ESI) m/z 1497.4; [M+H]⁺ calcd for C₇₂H₈₅ClF₃N₁₂O₁₂S₃⁺: 1497.52.

### Example 13: Preparation of Compound #17

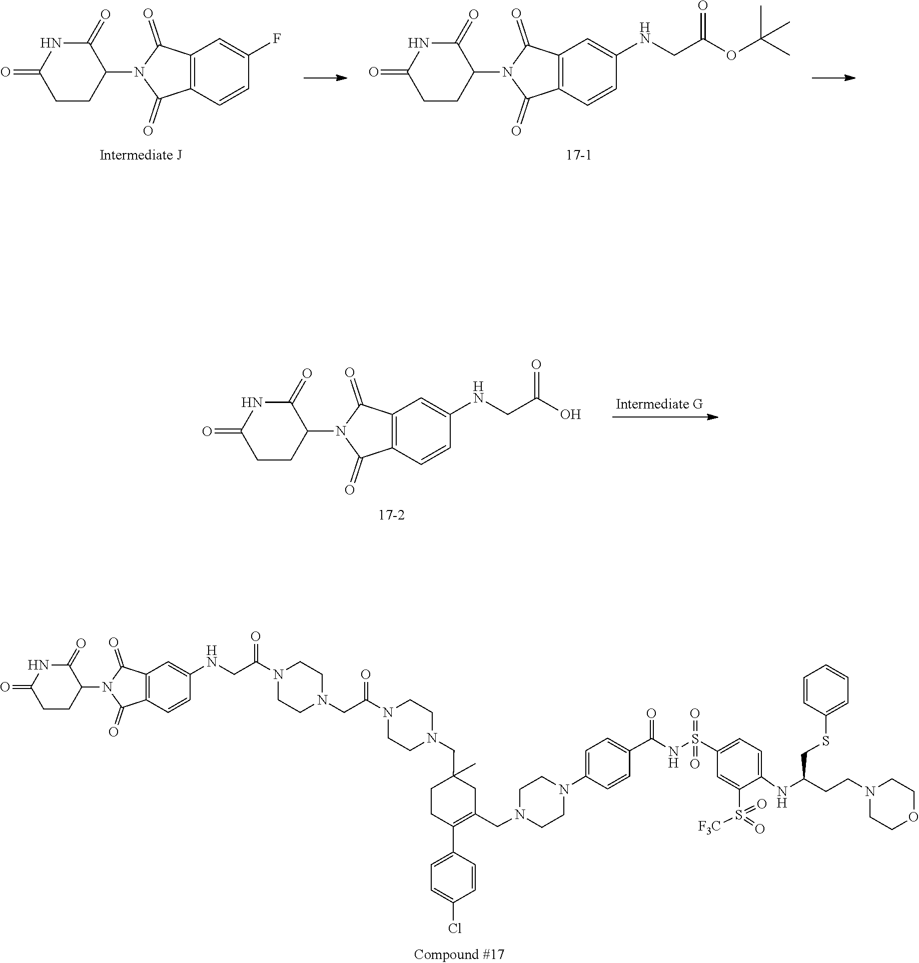 [SMILES: O=C1CCC(N2C(=O)c3ccc(F)cc3C2=O)C(=O)N1 ; CC(C)(C)OC(=O)CNc1ccc2c(c1)C(=O)N(C1CCC(=O)NC1=O)C2=O ; O=C(O)CNc1ccc2c(c1)C(=O)N(C1CCC(=O)NC1=O)C2=O ; CC1(CN2CCN(C(=O)CN3CCN(C(=O)CNc4ccc5c(c4)C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

### Compound #17 was Prepared by Following General Procedure G:

tert-butyl(2-(2,6-dioxopiperidin-3-yl)-1,3-dioxoisoindolin-5-yl)glycinate (17-1) (510 mg, 73% yield). ¹H NMR (600 MHZ, Chloroform-d) δ 8.11 (t, J=29.3 Hz, 1H), 7.64 (dt, J=8.3, 2.6 Hz, 1H), 6.95 (t, J=1.6 Hz, 1H), 6.77 (ddd, J=8.3, 2.2, 1.2 Hz, 1H), 4.93 (dd, J=12.5, 5.4 Hz, 1H), 3.89 (dd, J=4.9, 1.2 Hz, 2H), 2.92-2.68 (m, 3H), 2.16-2.08 (m, 1H), 1.51 (d, J=1.0 Hz, 9H). LC/MS (ESI) m/z 388.1; [M+H]⁺ calcd for C₁₉H₂₂N₃O₆⁺: 388.15.

(2-(2,6-dioxopiperidin-3-yl)-1,3-dioxoisoindolin-5-yl)glycine (17-2) (3.8 mg, quantitative yield). LC/MS (ESI) m/z 332.1; [M+H]⁺ calcd for C₁₅H₁₄N₃O₆⁺: 332.09.

4-(4-((4′-chloro-4-((4-(2-(4-((2-(2,6-dioxopiperidin-3-yl)-1,3-dioxoisoindolin-5-yl) glycyl) piperazin-1-yl) acetyl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-((trifluoromethyl) sulfonyl)phenyl) sulfonyl)benzamide (compound #17) (4.12 mg, 37% yield). ¹H NMR (600 MHZ, Chloroform-d) δ 8.31 (d, J=2.3 Hz, 1H), 8.09 (dd, J=9.3, 2.3 Hz, 1H), 7.67 (d, J=8.6 Hz, 2H), 7.61 (d, J=8.3 Hz, 1H), 7.39-7.32 (m, 2H), 7.32-7.21 (m, 6H), 7.00-6.96 (m, 2H), 6.93 (d, J=2.1 Hz, 1H), 6.85 (d, J=8.4 Hz, 1H), 6.74 (d, J=8.6 Hz, 2H), 6.64-6.56 (m, 1H), 4.92 (dd, J=12.4, 5.4 Hz, 1H), 3.94 (s, 2H), 3.92-3.84 (m, 1H), 3.70-3.48 (m, 11H), 3.47-3.38 (m, 2H), 3.28-3.16 (m, 6H), 3.09 (dd, J=13.9, 5.1 Hz, 1H), 3.02 (dd, J=13.9, 7.1 Hz, 1H), 2.93-2.67 (m, 5H), 2.64-2.16 (m, 20H), 2.15-2.05 (m, 2H), 1.92 (d, J=17.1 Hz, 1H), 1.74-1.49 (m, 4H), 1.47-1.37 (m, 1H), 0.95 (s, 3H). LC/MS (ESI) m/z 1497.4; [M+H]⁺ calcd for C₇₂H₈₅ClF₃N₁₂O₁₂S₃⁺: 1497.52.

### Example 14: Preparation of Compound #18

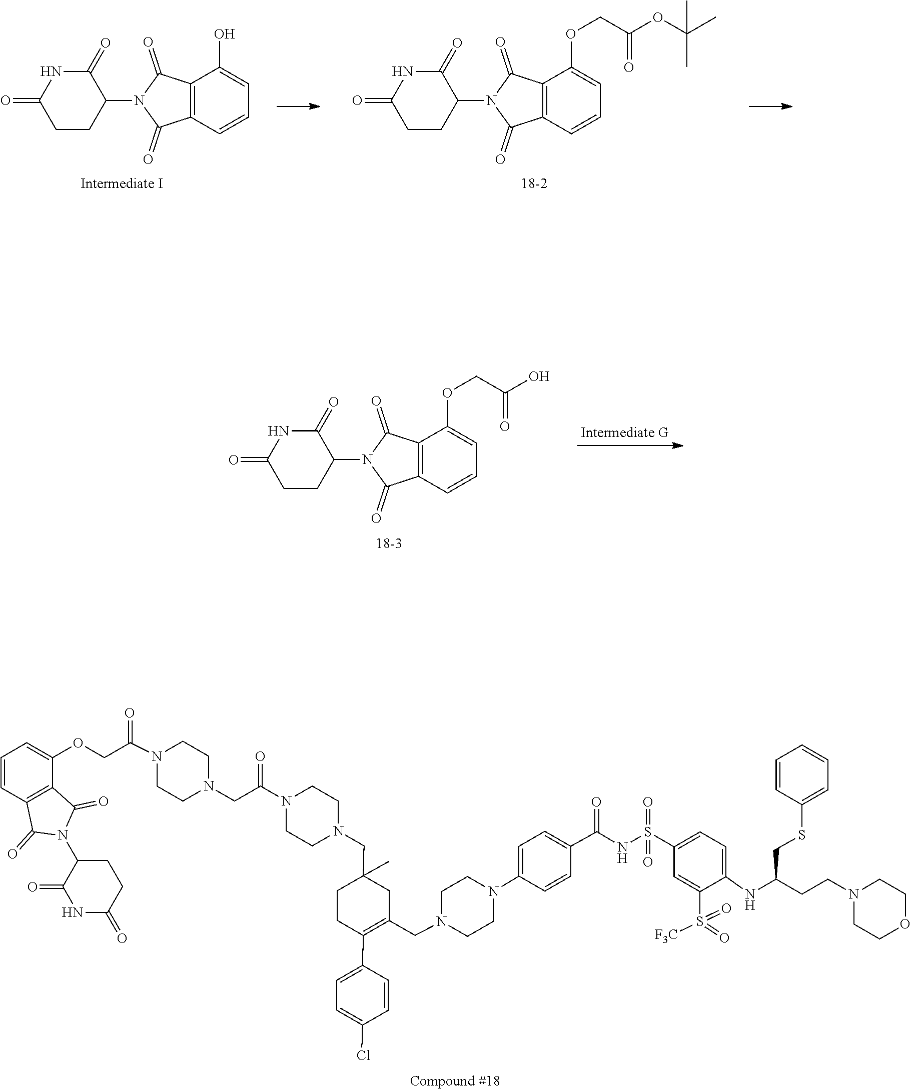 [SMILES: O=C1CCC(N2C(=O)c3cccc(O)c3C2=O)C(=O)N1 ; CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O ; CC1(CN2CCN(C(=O)CN3CCN(C(=O)COc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

### Compound #18 was Prepared by Following General Procedure G:

tert-butyl 2-((2-(2,6-dioxopiperidin-3-yl)-1,3-dioxoisoindolin-4-yl)oxy) acetate (18-1) (190 mg, 67% yield). ¹H NMR (600 MHZ, Chloroform-d) δ 8.01 (s, 1H), 7.66 (ddd, J=8.5, 7.3, 1.6 Hz, 1H), 7.50 (d, J=7.2 Hz, 1H), 7.10 (d, J=8.5 Hz, 1H), 4.97 (dd, J=12.3, 5.4 Hz, 1H), 4.78 (s, 2H), 2.92-2.70 (m, 3H), 2.16-2.07 (m, 1H), 1.47 (s, 9H). LC/MS (ESI) m/z 389.2; [M+H]⁺ calcd for C₁₉H₂₁N₂O₇+: 389.13.

2-((2-(2,6-dioxopiperidin-3-yl)-1,3-dioxoisoindolin-4-yl)oxy) acetic acid (18-2) (4.3 mg, quantitative yield). LC/MS (ESI) m/z 333.1; [M+H]⁺ calcd for C₁₅H₁₃N₂O₇+: 333.07.

4-(4-((4′-chloro-4-((4-(2-(4-(2-((2-(2,6-dioxopiperidin-3-yl)-1,3-dioxoisoindolin-4-yl)oxy) acetyl) piperazin-1-yl) acetyl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-((trifluoromethyl) sulfonyl)phenyl) sulfonyl)benzamide (compound #18) (1.81 mg, 16% yield). ¹H NMR (600 MHZ, Chloroform-d) δ 8.33 (d, J=6.3, 2.2 Hz, 1H), 8.07 (t, J=7.3 Hz, 1H), 7.73 (t, J=7.7 Hz, 2H), 7.64-7.57 (m, 1H), 7.49-7.42 (m, 1H), 7.37 (d, J=7.6 Hz, 2H), 7.34-7.21 (m, 6H), 7.06 (dd, J=8.4, 4.1 Hz, 1H), 7.03-6.94 (m, 3H), 6.75 (s, 2H), 6.61 (d, J=9.3 Hz, 1H), 4.94 (s, 1H), 4.77 (d, J=5.1 Hz, 2H), 3.89 (s, 1H), 3.73-3.45 (m, 10H), 3.45-3.05 (m, 10H), 3.05-2.79 (m, 4H), 2.78-2.69 (m, 1H), 2.69-2.16 (m, 20H), 2.15-2.07 (m, 2H), 1.94-1.80 (m, 1H), 1.73-1.53 (m, 4H), 1.51-1.39 (m, 1H), 0.96 (s, 3H). LC/MS (ESI) m/z 1498.1; [M+H]⁺ calcd for C₇₂H₈₄ClF₃N₁₁O₁₃S₃⁺: 1498.05.

### Example 15: Preparation of Compounds #19-21

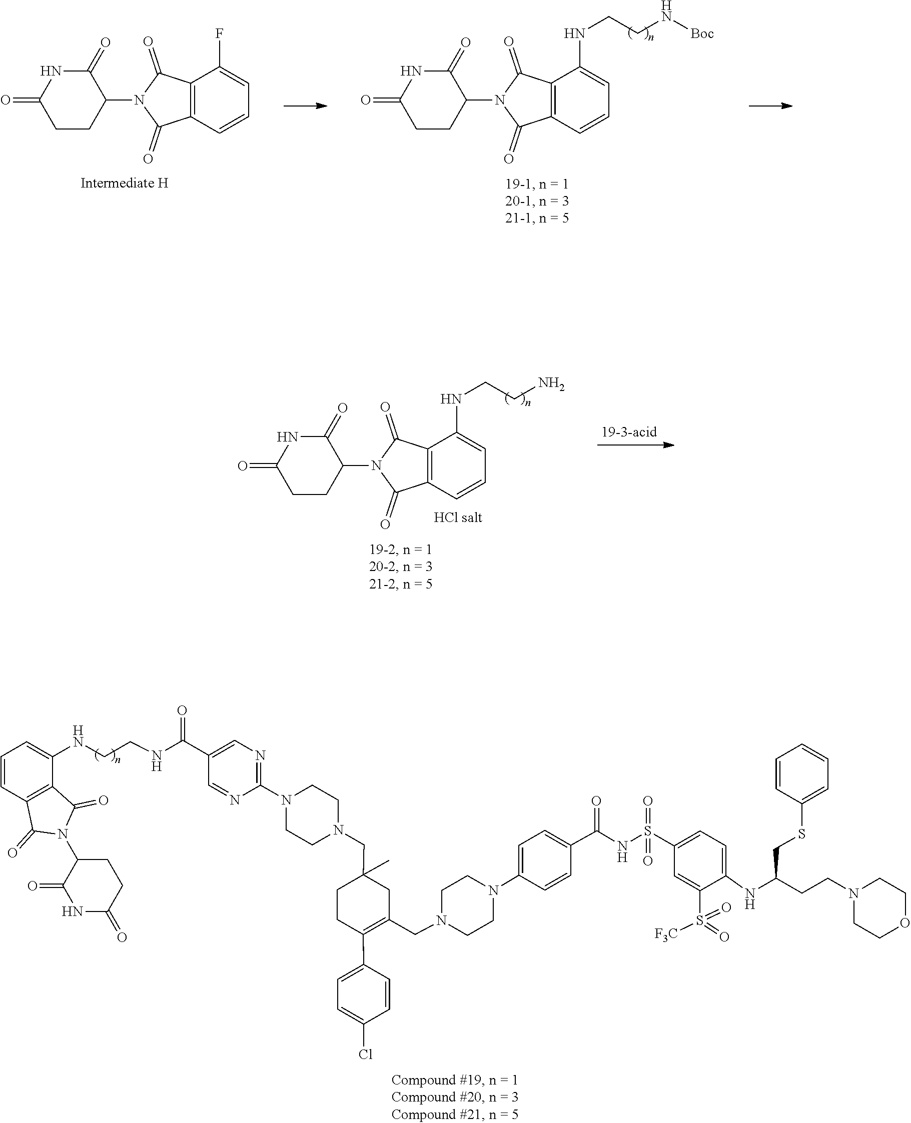

Compound #19-21 were Prepared by Following General Procedure G:

tert-butyl(2-((2-(2,6-dioxopiperidin-3-yl)-1,3-dioxoisoindolin-4-yl)amino)ethyl) carbamate (19-1) (521 mg, 69% yield). ¹H NMR (600 MHZ, Chloroform-d) δ 8.03 (s, 1H), 7.50 (dd, J=8.5, 7.1 Hz, 1H), 7.12 (d, J=7.1 Hz, 1H), 6.99 (d, J=8.5 Hz, 1H), 6.39 (t, J=6.0 Hz, 1H), 4.92 (dd, J=12.4, 5.3 Hz, 1H), 4.82 (s, 1H), 3.45 (q, J=6.1 Hz, 2H), 3.36 (q, J=6.2 Hz, 2H), 2.93-2.86 (m, 1H), 2.85-2.69 (m, 2H), 2.17-2.08 (m, 1H), 1.45 (s, 9H). LC/MS (ESI) m/z 417.3; [M+H]⁺ calcd for C₂₀H₂₅N₄O₆⁺: 417.18.

tert-butyl (4-((2-(2,6-dioxopiperidin-3-yl)-1,3-dioxoisoindolin-4-yl)amino)butyl) carbamate (20-1) (443 mg, 55% yield). ¹H NMR (600 MHZ, Chloroform-d) δ 8.23 (s, 1H), 7.48 (ddd, J=8.6, 7.1, 1.8 Hz, 1H), 7.09 (dd, J=7.1, 1.9 Hz, 1H), 6.88 (d, J=8.6 Hz, 1H), 6.23 (t, J=5.7 Hz, 1H), 4.91 (dd, J=12.4, 5.3 Hz, 1H), 4.59 (s, 1H), 3.29 (q, J=6.6 Hz, 2H), 3.17 (q, J=6.8 Hz, 2H), 2.93-2.84 (m, 1H), 2.84-2.66 (m, 2H), 2.17-2.07 (m, 1H), 1.74-1.56 (m, 4H), 1.44 (s, 9H). LC/MS (ESI) m/z 445.2; [M+H]⁺ calcd for C₂₂H₂₉N₄O₆⁺: 445.21.

tert-butyl (6-((2-(2,6-dioxopiperidin-3-yl)-1,3-dioxoisoindolin-4-yl)amino) hexyl) carbamate (21-1) (617 mg, 72% yield). ¹H NMR (600 MHZ, Chloroform-d) δ 8.07 (s, 1H), 7.49 (dd, J=8.5, 7.1 Hz, 1H), 7.09 (d, J=7.1 Hz, 1H), 6.87 (d, J=8.5 Hz, 1H), 6.22 (t, J=5.7 Hz, 1H), 4.91 (dd, J=12.3, 5.4 Hz, 1H), 4.52 (s, 1H), 3.26 (q, J=7.0, 5.6 Hz, 2H), 3.12 (q, J=6.8 Hz, 2H), 2.93-2.85 (m, 1H), 2.85-2.67 (m, 2H), 2.17-2.09 (m, 1H), 1.66 (p, J=7.2 Hz, 2H), 1.49 (q, J=7.4 Hz, 2H), 1.47-1.40 (m, 11H), 1.40-1.34 (m, 2H). LC/MS (ESI) m/z 473.2; [M+H]⁺ calcd for C₂₄H₃₃N₄O₆⁺: 473.21.

4-((2-aminoethyl)amino)-2-(2,6-dioxopiperidin-3-yl) isoindoline-1,3-dione hydrochloride 8 (19-2) (5.1 mg, quantitative yield). LC/MS (ESI) m/z 317.2; [M+H]⁺ calcd for C₁₅H₁₇N₄O₄⁺: 317.12.

4-((4-aminobutyl)amino)-2-(2,6-dioxopiperidin-3-yl) isoindoline-1,3-dione hydrochloride 12 (20-2) (6.5 mg, quantitative yield) . . . . LC/MS (ESI) m/z 345.2; [M+H]⁺ calcd for C₁₇H₂₁N₄O₄⁺: 345.16.

4-((6-aminohexyl)amino)-2-(2,6-dioxopiperidin-3-yl) isoindoline-1,3-dione hydrochloride (21-2) (6.5 mg, quantitative yield). LC/MS (ESI) m/z 373.3; [M+H]⁺ calcd for C₁₉H₂₅N₄O₄⁺: 373.19.

The preparation of 19-3-acid was performed according to patent WO 2023107606 A1. 2-(4-((4′-chloro-4-methyl-6-((4-(4-(((4-(((R)-4-morpholino-1-(phenylthio) butan-2-yl)amino)-3-((trifluoromethyl) sulfonyl)phenyl) sulfonyl) carbamoyl)phenyl) piperazin-1-yl)methyl)-2,3,4,5-tetrahydro-[1,1′-biphenyl]-4-yl)methyl) piperazin-1-yl)-N-(2-((2-(2,6-dioxopiperidin-3-yl)-1,3-dioxoisoindolin-4-yl)amino)ethyl)pyrimidine-5-carboxamide (compound #19) (2.89 mg, 17% yield). ¹H NMR (600 MHZ, Chloroform-d) δ 8.79 (s, 1H), 8.65 (s, 2H), 8.33 (d, J=2.3 Hz, 1H), 8.08 (d, J=9.1 Hz, 1H), 7.68 (d, J=8.5 Hz, 2H), 7.43 (dd, J=8.7, 7.0 Hz, 1H), 7.38-7.32 (m, 2H), 7.32-7.19 (m, 5H), 7.00 (ddd, J=31.3, 13.1, 7.9 Hz, 4H), 6.89 (s, 1H), 6.73 (d, J=8.6 Hz, 2H), 6.60 (d, J=9.4 Hz, 1H), 6.42 (d, J=6.8 Hz, 1H), 4.95-4.84 (m, 1H), 3.96-3.76 (m, 5H), 3.72-3.57 (m, 7H), 3.53 (s, 2H), 3.25 (s, 4H), 3.09 (dd, J=13.8, 5.0 Hz, 1H), 3.01 (dd, J=13.9, 7.1 Hz, 1H), 2.92 (s, 2H), 2.84 (d, J=14.0 Hz, 1H), 2.80-2.65 (m, 2H), 2.60 (s, 4H), 2.53-2.17 (m, 15H), 2.15-2.03 (m, 2H), 2.01-1.86 (m, 1H), 1.73-1.57 (m, 3H), 1.50-1.38 (m, 1H), 0.97 (s, 3H). LC/MS (ESI) m/z 1478.7; [M+H]⁺ calcd for C₇₁H₈₀ClF₃N₁₃O₁₁S₃⁺: 1478.49.

2-(4-((4′-chloro-4-methyl-6-((4-(4-(((4-(((R)-4-morpholino-1-(phenylthio) butan-2-yl)amino)-3-((trifluoromethyl) sulfonyl)phenyl) sulfonyl) carbamoyl)phenyl) piperazin-1-yl)methyl)-2,3,4,5-tetrahydro-[1,1′-biphenyl]-4-yl)methyl) piperazin-1-yl)-N-(4-((2-(2,6-dioxopiperidin-3-yl)-1,3-dioxoisoindolin-4-yl)amino)butyl)pyrimidine-5-carboxamide (compound #20) (3.71 mg, 21% yield). ¹H NMR (600 MHz, Chloroform-d) δ 8.66 (s, 2H), 8.34 (d, J=2.3 Hz, 1H), 8.09 (dd, J=9.3, 2.3 Hz, 1H), 7.67 (d, J=8.4 Hz, 2H), 7.45 (q, J=8.1 Hz, 1H), 7.36 (d, J=7.2 Hz, 2H), 7.32-7.21 (m, 5H), 7.06 (d, J=7.1 Hz, 1H), 7.03 (d, J=8.4 Hz, 1H), 6.99 (d, J=8.3 Hz, 2H), 6.86 (d, J=8.6 Hz, 1H), 6.73 (d, J=8.6 Hz, 2H), 6.60 (d, J=9.3 Hz, 1H), 6.27 (s, 1H), 6.23 (t, J=5.8 Hz, 1H), 4.95-4.85 (m, 1H), 3.94-3.77 (m, 5H), 3.72-3.58 (m, 4H), 3.53-3.39 (m, 2H), 3.30 (d, J=5.8 Hz, 2H), 3.28-3.17 (m, 4H), 3.10 (dd, J=13.9, 5.0 Hz, 1H), 3.01 (dd, J=13.9, 7.1 Hz, 1H), 2.94-2.80 (m, 3H), 2.80-2.66 (m, 2H), 2.60 (p, J=6.0 Hz, 4H), 2.52-2.18 (m, 16H), 2.18-2.03 (m, 2H), 1.93 (d, J=17.2 Hz, 1H), 1.77-1.59 (m, 7H), 1.52-1.40 (m, 1H), 0.97 (s, 3H). LC/MS (ESI) m/z 1506.6; [M+H]⁺ calcd for C₇₃H₈₄ClF₃N₁₃O₁₁S₃⁺: 1506.52.

2-(4-((4′-chloro-4-methyl-6-((4-(4-(((4-(((R)-4-morpholino-1-(phenylthio) butan-2-yl)amino)-3-((trifluoromethyl) sulfonyl)phenyl) sulfonyl) carbamoyl)phenyl) piperazin-1-yl)methyl)-2,3,4,5-tetrahydro-[1,1′-biphenyl]-4-yl)methyl) piperazin-1-yl)-N-(6-((2-(2,6-dioxopiperidin-3-yl)-1,3-dioxoisoindolin-4-yl)amino) hexyl)pyrimidine-5-carboxamide (compound #21) (3.25 mg, 18% yield). ¹H NMR (600 MHZ, Chloroform-d) δ 8.90 (s, 1H), 8.67 (s, 2H), 8.31 (d, J =2.2 Hz, 1H), 8.07 (d, J=8.5 Hz, 1H), 7.70 (d, J=8.5 Hz, 2H), 7.46 (dd, J=8.5, 7.1 Hz, 1H), 7.35 (d, J=7.5 Hz, 2H), 7.32-7.18 (m, 5H), 7.05 (d, J=7.1 Hz, 1H), 6.99 (d, J=8.0 Hz, 2H), 6.86 (d, J=8.6 Hz, 1H), 6.71 (d, J=8.6 Hz, 2H), 6.59 (d, J=9.4 Hz, 1H), 6.47 (s, 1H), 6.21 (t, J=5.6 Hz, 1H), 4.88 (ddd, J=12.4, 5.4, 2.3 Hz, 1H), 4.03-3.75 (m, 4H), 3.74-3.58 (m, 4H), 3.46-3.28 (m, 6H), 3.28-3.20 (m, 2H), 3.09 (dd, J=13.9, 5.0 Hz, 1H), 3.01 (dd, J=13.9, 7.0 Hz, 1H), 2.88-2.80 (m, 1H), 2.80-2.60 (m, 6H), 2.60-2.18 (m, 16H), 2.18-2.01 (m, 2H), 1.92-1.51 (m, 10H), 1.51-1.34 (m, 6H), 0.99 (s, 3H). LC/MS (ESI) m/z 1534.6; [M+H]⁺ calcd for C₇₅H₈₈ClF₃N₁₃O₁₁S₃⁺: 1534.55.

### Example 16: Preparation of Compound #22

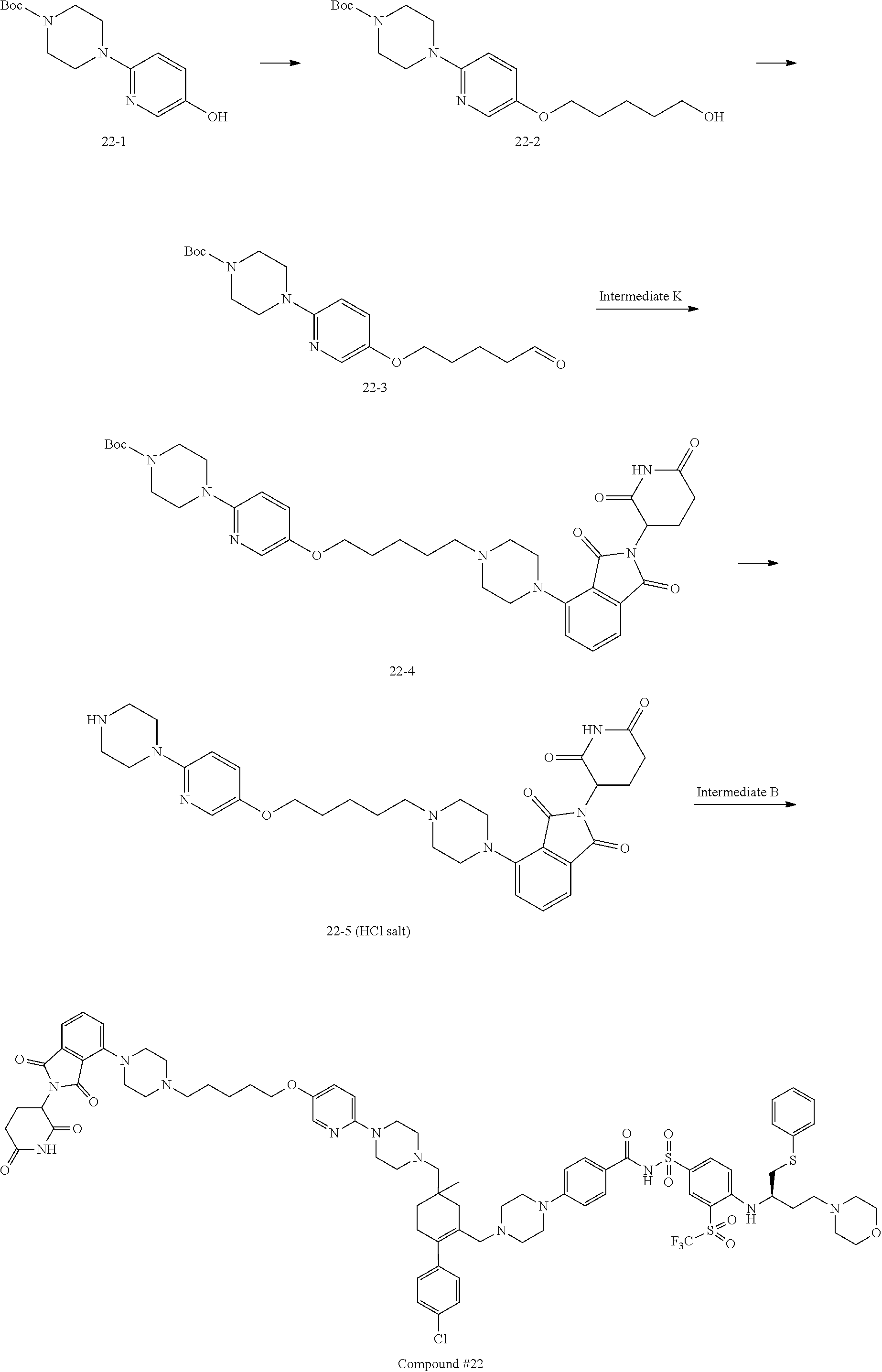 [SMILES: CC(C)(C)OC(=O)N1CCN(c2ccc(O)cn2)CC1 ; CC(C)(C)OC(=O)N1CCN(c2ccc(OCCCCCO)cn2)CC1 ; CC(C)(C)OC(=O)N1CCN(c2ccc(OCCCCC=O)cn2)CC1 ; CC(C)(C)OC(=O)N1CCN(c2ccc(OCCCCCN3CCN(c4cccc5c4C(=O)N(C4CCC(=O)NC4=O)C5=O)CC3)cn2)CC1 ; O=C1CCC(N2C(=O)c3cccc(N4CCN(CCCCCOc5ccc(N6CCNCC6)nc5)CC4)c3C2=O)C(=O)N1 ; CC1(CN2CCN(c3ccc(OCCCCCN4CCN(c5cccc6c5C(=O)N(C5CCC(=O)NC5=O)C6=O)CC4)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

Step 1: Synthesis of tert-butyl 4-(5-((5-hydroxypentyl)oxy) pyridin-2-yl) piperazine-1-carboxylate (22-2). A mixture of 22-1 (410 mg, 1.47 mmol), N-Boc-piperazine (491 mg, 2.94 mmol) and K₂CO₃ (608 mg, 4.41 mmol) in DMF (9.0 mL) was stirred at 80° C. overnight. The reaction mixture was diluted with EtOAc and washed with water and sat. aq. NH₄Cl. The organic layers were dried over Na₂SO₄, filtered and concentrated. The residue was purified by flash column chromatography (0% to 60% of EtOAc in hexane) to afford 22-2 (160 mg, 30% yield). 1H NMR (600 MHZ, Chloroform-d) δ 7.92 (d, J=3.0 Hz, 1H), 7.14 (dd, J=9.0, 3.0 Hz, 1H), 6.63 (d, J=9.1 Hz, 1H), 3.94 (t, J=6.4 Hz, 2H), 3.67 (t, J=6.5 Hz, 2H), 3.54 (t, J=5.2 Hz, 4H), 3.39-3.36 (m, 4H), 1.82-1.76 (m, 2H), 1.66-1.60 (m, 2H), 1.57-1.50 (m, 2H), 1.48 (s, 9H). LC/MS (ESI) m/z 366.3; [M+H]⁺ calcd for C₁₉H₃₂N₃O₄⁺: 366.24.

Step 2: Synthesis of tert-butyl 4-(5-((5-oxopentyl)oxy) pyridin-2-yl) piperazine-1-carboxylate (22-3). A mixture of 22-2 (30 mg, 0.082 mmol) and Dess-Martin periodinane (45 mg, 0.11 mmol) in EtOAc (1 mL) was stirred at room temperature for 1 h. The resulting mixture was filtered and concentrated to afford crude 22-3 (35 mg, quantitative yield), which was directly used in the next step. LC/MS (ESI) m/z 364.2; [M+H]⁺ calcd for C₁₉H₃₀N₃O₄⁺: 364.22.

Step 3: Synthesis of tert-butyl 4-(5-((5-(4-(2-(2,6-dioxopiperidin-3-yl)-1,3-dioxoisoindolin-4-yl) piperazin-1-yl) pentyl)oxy) pyridin-2-yl) piperazine-1-carboxylate (22-4). 22-3 (35 mg, crude) and NaBH(OAc)₃ (50 mg, 0.24 mmol) were added to a solution of Intermediate K (30 mg, 0.079 mmol) and TEA (55 μL, 0.4 mmol) in DCM (2 mL), then stirred at room temperature overnight. The resulting mixture was washed with sat. aq. NH₄Cl. The organic layers were dried over Na₂SO₄, filtered and concentrated. The residue was purified by flash column chromatography (0% to 5% of MeOH in DCM) to afford 22-4 (21 mg, 37% yield in two steps).

1H NMR (600 MHZ, Chloroform-d) δ 8.40 (s, 1H), 7.91 (d, J=3.1 Hz, 1H), 7.62 (dd, J=8.4, 7.2 Hz, 1H), 7.47-7.43 (m, 1H), 7.19 (d, J=8.1 Hz, 1H), 7.14 (dd, J=9.1, 3.1 Hz, 1H), 6.62 (d, J=9.1 Hz, 1H), 4.95 (dd, J=12.4, 5.4 Hz, 1H), 3.93 (t, J=6.2 Hz, 2H), 3.61 (t, J=5.1 Hz, 4H), 3.54 (t, J=5.3 Hz, 4H), 3.39-3.35 (m, 4H), 3.15 (s, 4H), 2.91-2.67 (m, 5H), 2.18-2.08 (m, 1H), 1.89-1.75 (m, 4H), 1.59-1.50 (m, 2H), 1.47 (s, 9H). LC/MS (ESI) m/z 690.4; [M+H]⁺ calcd for C₃₆H₄₈N—O₇⁺: 690.36.

Step 4: Synthesis of 2-(2,6-dioxopiperidin-3-yl)-4-(4-(5-((6-(piperazin-1-yl)pyridin-3-yl)oxy) pentyl) piperazin-1-yl) isoindoline-1,3-dione hydrochloride (22-5). HCl (1 mL, 4 N HCl in dioxane) was added to a stirring solution of 22-4 (20 mg, 0.029 mmol) in DCM (1 mL) and stirred at room temperature for 2 hours. The reaction mixture was concentrated to afford 22-5 (19 mg, quantitative yield). LC/MS (ESI) m/z 590.4; [M+H]⁺ calcd for C₃₁H₄₀N₇O₅⁺: 590.31.

Step 5: Synthesis of 4-(4-((4′-chloro-4-((4-(5-((5-(4-(2-(2,6-dioxopiperidin-3-yl)-1,3-dioxoisoindolin-4-yl) piperazin-1-yl) pentyl)oxy) pyridin-2-yl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-22 ((trifluoromethyl) sulfonyl)phenyl) sulfonyl)benzamide (compound #22). Intermediate B (19 mg, 0.019 mmol) and NaBH(OAc)₃ (5 mg, 0.023 mmol) were added to a solution of 22-5 (19 mg, 0.03 mmol) and TEA (16 μL, 0.12 mmol) in DCM (1 mL), then stirred at room temperature overnight. The resulting mixture was washed with sat. aq. NH₄Cl and dried over Na₂SO₄, filtered and concentrated. The residue was purified by preparative TLC (DCM/MeOH) to afford compound #22 (2.24 mg, 7% yield). ¹H NMR (600 MHZ, Chloroform-d) δ 8.34 (d, J=2.2 Hz, 1H), 8.27 (s, 1H), 8.04 (dd, J=9.3, 2.2 Hz, 1H), 7.88 (d, J=3.0 Hz, 1H), 7.76 (d, J=8.7 Hz, 2H), 7.61 (t, J=8.4, 7.2 Hz, 1H), 7.44 (d, J=7.2 Hz, 1H), 7.40-7.34 (m, 2H), 7.33-7.21 (m, 5H), 7.19 (d, J=8.4 Hz, 1H), 7.08 (dd, J=9.1, 3.1 Hz, 1H), 7.02-6.96 (m, 2H), 6.94 (d, J=8.6 Hz, 1H), 6.75 (d, J=8.7 Hz, 2H), 6.55 (d, J=9.3 Hz, 1H), 6.53 (d, J=9.1 Hz, 1H), 4.95 (dd, J=12.4, 5.4 Hz, 1H), 3.92 (t, J=6.3 Hz, 2H), 3.90-3.82 (m, 1H), 3.71-3.61 (m, 4H), 3.57 (s, 4H), 3.36 (s, 4H), 3.24-3.19 (m, 4H), 3.16-3.03 (m, 4H), 2.98 (dd, J=13.8, 7.4 Hz, 1H), 2.93-2.63 (m, 11H), 2.50-2.19 (m, 15H), 2.18-2.07 (m, 2H), 1.88 (d, J=17.4 Hz, 1H), 1.82-1.70 (m, 4H), 1.70-1.56 (m, 2H), 1.55-1.46 (m, 2H), 1.46-1.39 (m, 1H), 0.97 (s, 3H). 2 LC/MS (ESI) m/z 1561.7; [M+H]⁺ calcd for C₇₈H₉₃ClF₃N₁₂O₁₁S₃⁺: 1561.59.

### Example 17: Preparation of Compound #23

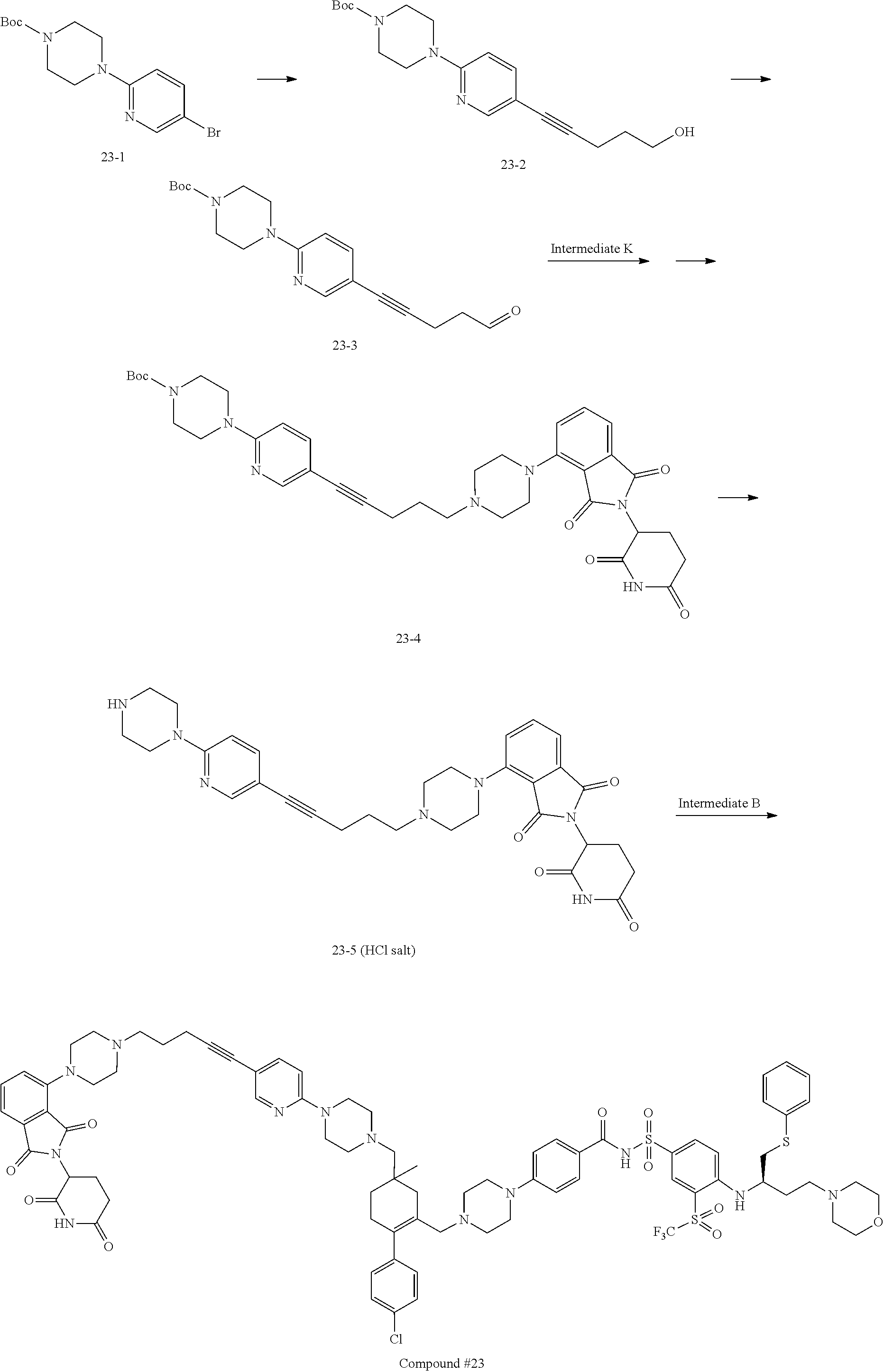 [SMILES: CC(C)(C)OC(=O)N1CCN(c2ccc(Br)cn2)CC1 ; CC(C)(C)OC(=O)N1CCN(c2ccc(C#CCCCO)cn2)CC1 ; CC(C)(C)OC(=O)N1CCN(c2ccc(C#CCCC=O)cn2)CC1 ; CC(C)(C)OC(=O)N1CCN(c2ccc(C#CCCCN3CCN(c4cccc5c4C(=O)N(C4CCC(=O)NC4=O)C5=O)CC3)cn2)CC1 ; O=C1CCC(N2C(=O)c3cccc(N4CCN(CCCC#Cc5ccc(N6CCNCC6)nc5)CC4)c3C2=O)C(=O)N1 ; CC1(CN2CCN(c3ccc(C#CCCCN4CCN(c5cccc6c5C(=O)N(C5CCC(=O)NC5=O)C6=O)CC4)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

Step 1: Synthesis of tert-butyl 4-(5-(5-hydroxypent-1-yn-1-yl)pyridin-2-yl) piperazine-1-carboxylate (23-2). Under an argon atmosphere, a mixture of 23-1 (100 mg, 0.29 mmol), pent-4-yn-1-ol (123 mg, 1.46 mmol), PdCl2 (PPh3)₂ (10.3 mg, 0.015 mmol), CuI (5.6 mg, 0.029 mmol) and TEA (0.5 mL) in DMSO (3 mL) was stirred at 80° C. overnight. After cooling to room temperature, the reaction mixture was diluted with EtOAc, then washed with water and sat. aq.

NH₄Cl. The organic layers were dried over Na₂SO₄, filtered and concentrated. The residue was purified by flash column chromatography (0% to 60% of EtOAc in hexanes) to afford 23-2 (60 mg, 59% yield). ¹H NMR (600 MHZ, Chloroform-d) δ 8.20 (d, J=2.3, 0.7 Hz, 1H), 7.46 (dd, J=8.8, 2.3 Hz, 1H), 6.52 (d, J=8.8, 0.8 Hz, 1H), 3.79 (t, J=6.2 Hz, 2H), 3.55-3.49 (m, 8H), 2.52 (t, J=6.9 Hz, 2H), 1.84 (p, 2H), 1.47 (s, 9H). LC/MS (ESI) m/z 346.3; [M+H]⁺ calcd for C₁₉H₂₈N₃O₃⁺: 346.21.

Step 2: Synthesis of tert-butyl 4-(5-(5-oxopent-1-yn-1-yl)pyridin-2-yl) piperazine-1-carboxylate (23-3). A mixture of 23-2 (33 mg, 0.096 mmol) and Dess-Martin periodinane (49 mg, 0.11 mmol) in EtOAc (1 mL) was stirred at room temperature for 1 h. The resulting mixture was filtered and concentrated to afford crude 23-3 (41 mg, quantitative yield), which was directly used in the next step. LC/MS (ESI) m/z 344.2; [M+H]⁺ calcd for C₁₉H₂₆N₃O₃⁺: 344.20.

Step 3: Synthesis of tert-butyl 4-(5-(5-(4-(2-(2,6-dioxopiperidin-3-yl)-1,3-dioxoisoindolin-4-yl) piperazin-1-yl) pent-1-yn-1-yl)pyridin-2-yl) piperazine-1-carboxylate (23-4). 23-3 (41 mg, crude) and NaBH(OAc)₃ (75 mg, 0.24 mmol) were added to a solution of Intermediate K (30 mg, 0.079 mmol) and TEA (55 μL, 0.4 mmol) in DCM (2 mL), then stirred at room temperature overnight. The resulting mixture was washed with sat. aq. NH₄Cl. The organic layers were dried over Na₂SO₄, filtered and concentrated. The residue was purified by flash column chromatography (0% to 5% of MeOH in DCM) to afford 23-4 (25 mg, 39% yield in two steps). 1H NMR (600 MHZ, Chloroform-d) δ 8.44 (s, 1H), 8.21 (dd, J=2.3, 0.7 Hz, 1H), 7.58 (dd, J=8.4, 7.2 Hz, 1H), 7.46 (dd, J=8.8, 2.3 Hz, 1H), 7.39 (dd, J=7.2, 0.7 Hz, 1H), 7.18-7.14 (m, 1H), 6.54 (dd, J=8.8, 0.9 Hz, 1H), 4.95 (dd, J=12.5, 5.4 Hz, 1H), 3.59-3.46 (m, 8H), 3.44-3.28 (m, 4H), 2.91-2.76 (m, 2H), 2.76-2.64 (m, 5H), 2.57 (t, J=7.4 Hz, 2H), 2.46 (t, J=7.0 Hz, 2H), 2.13-2.06 (m, 1H), 1.82 (p, J=7.2 Hz, 2H), 1.47 (s, 9H). LC/MS (ESI) m/z 670.2; [M+H]⁺ calcd for C₃₆H₄₄N₇O₆⁺: 670.33.

Step 4: Synthesis of 2-(2,6-dioxopiperidin-3-yl)-4-(4-(5-(6-(piperazin-1-yl)pyridin-3-yl) pent-4-yn-1-yl) piperazin-1-yl) isoindoline-1,3-dione hydrochloride (23-5). HCl (1 mL, 4 N HCl in dioxane) was added to a stirring solution of 23-4 (20 mg, 0.03 mmol) in DCM (1 mL) at room temperature. Then the resulting mixture was stirred at room temperature for 2 hours, then concentrated to afford 23-5 (23 mg, quantitative yield). LC/MS (ESI) m/z 570.3; [M+H]⁺ calcd for C₃₁H₃₆N₇O₄⁺: 570.28.

Step 5: Synthesis of 4-(4-((4′-chloro-4-((4-(5-(5-(4-(2-(2,6-dioxopiperidin-3-yl)-1,3-26 dioxoisoindolin-4-yl) piperazin-1-yl) pent-1-yn-1-yl)pyridin-2-yl) piperazin-1-yl)methyl)-4-methyl-3,4,5,6-tetrahydro-[1,1′-biphenyl]-2-yl)methyl) piperazin-1-yl)-N-((4-(((R)-4-morpholino-1-(phenylthio) butan-2-yl)amino)-3-28 ((trifluoromethyl) sulfonyl)phenyl) sulfonyl)benzamide (compound #23). Intermediate B (23 mg, 0.023 mmol) and NaBH(OAc)₃ (6 mg, 0.028 mmol) were added to a solution of 23-5 (23 mg, 0.038 mmol) and TEA (16 μL, 0.12 mmol) in DCM (1 mL), then stirred at room temperature overnight. The resulting mixture was washed with sat. aq. NH₄Cl. The organic layers were dried over Na₂SO₄, filtered and concentrated. The residue was purified by preparative TLC (DCM/MeOH) to afford compound #23 (3.89 mg, 11% yield). ¹H NMR (600 MHz, Chloroform-d) δ 8.37 (t, J=1.7 Hz, 1H), 8.33 (s, 1H), 8.22 (d, J=2.2 Hz, 1H), 8.09 (dd, J=9.2, 2.2 Hz, 1H), 7.74 (d, J=8.6 Hz, 2H), 7.62 (dd, J=8.4, 7.2 Hz, 1H), 7.48-7.42 (m, 2H), 7.42-7.37 (m, 2H), 7.35-7.24 (m, 5H), 7.20 (d, J=8.5 Hz, 1H), 7.05-6.97 (m, 3H), 6.77 (d, J=9.0 Hz, 2H), 6.59 (d, J=9.5 Hz, 1H), 6.52 (d, J=8.8 Hz, 1H), 4.98 (dd, J=12.5, 5.4 Hz, 1H), 3.94-3.85 (m, 1H), 3.73-3.62 (m, 4H), 3.59-3.43 (m, 8H), 3.26 (t, J=5.2 Hz, 4H), 3.11 (dd, J=13.9, 5.0 Hz, 1H), 3.02 (dd, J=13.9, 7.4 Hz, 1H), 2.99-2.90 (m, 4H), 2.90-2.79 (m, 5H), 2.79-2.62 (m, 5H), 2.51 (t, J=6.9 Hz, 2H), 2.49-2.22 (m, 13H), 2.19-2.08 (m, 2H), 1.99-1.87 (m, 3H), 1.73-1.61 (m, 3H), 1.52-1.44 (m, 1H), 0.99 (s, 3H). LC/MS (ESI) m/z 1541.6; [M+H]⁺ calcd for C₇₈H₈₉ClF₃N₁₂O₁₀S₃⁺: 1541.56.

### Example 18: Preparation of Compound #24

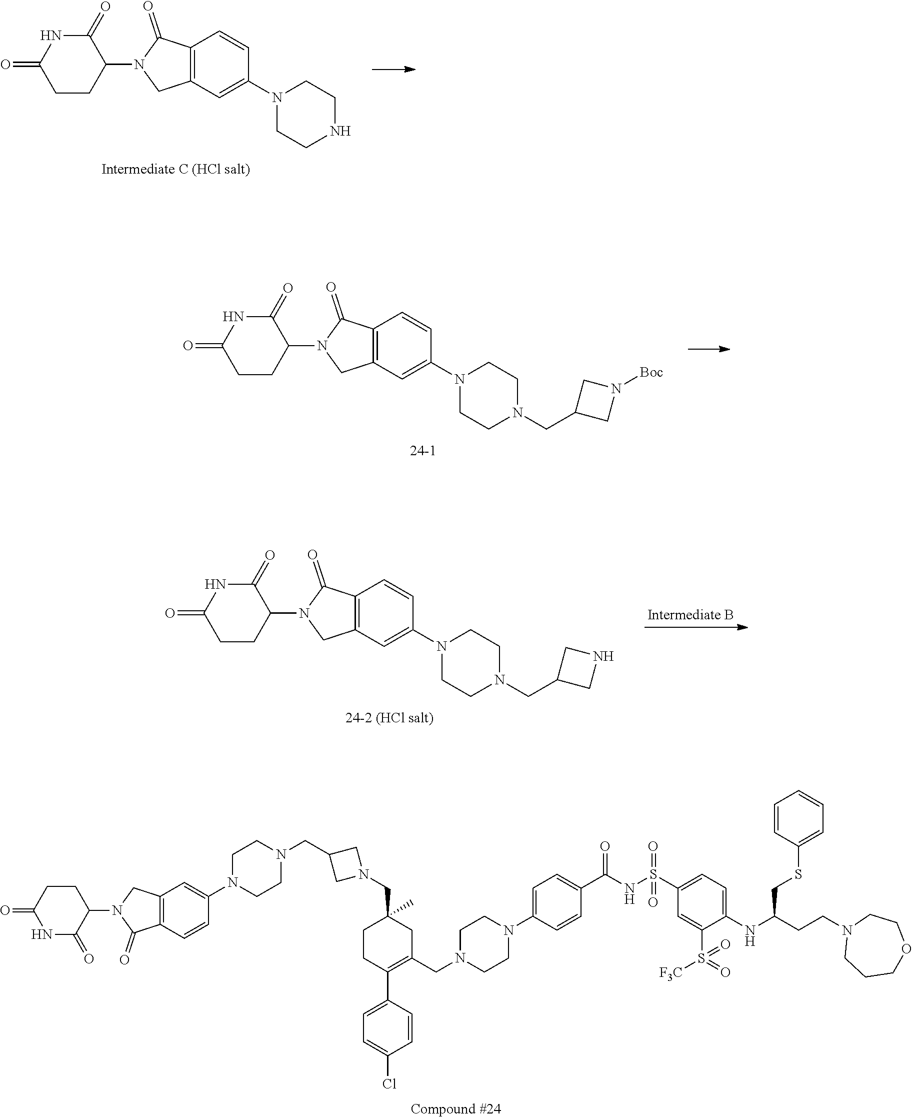 [SMILES: O=C1CCC(N2Cc3cc(N4CCNCC4)ccc3C2=O)C(=O)N1 ; CC(C)(C)OC(=O)N1CC(CN2CCN(c3ccc4c(c3)CN(C3CCC(=O)NC3=O)C4=O)CC2)C1 ; O=C1CCC(N2Cc3cc(N4CCN(CC5CNC5)CC4)ccc3C2=O)C(=O)N1 ; C[C@@]1(CN2CC(CN3CCN(c4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #24 was Prepared by Following General Procedure C:

tert-butyl 3-((4-(2-(2,6-dioxopiperidin-3-yl)-1-oxoisoindolin-5-yl) piperazin-1-yl)methyl) azetidine-1-carboxylate (24-1) (67 mg, 20% yield). ¹H NMR (600 MHZ, Chloroform-d) δ 8.06 (s, 1H), 7.75 (d, J=8.5 Hz, 1H), 7.00 (dd, J=8.6, 2.2 Hz, 1H), 6.89 (d, J=2.1 Hz, 1H), 5.21 (dd, J=13.3, 5.1 Hz, 1H), 4.43 (d, J=15.5 Hz, 1H), 4.27 (d, J=15.6 Hz, 1H), 4.06 (t, J=8.4 Hz, 2H), 3.64 (dd, J=8.6, 5.4 Hz, 2H), 3.32 (t, 4H), 2.92 (ddd, J=17.8, 4.8, 2.5 Hz, 1H), 2.84 (ddd, J=17.9, 13.3, 5.4 Hz, 1H), 2.81-2.74 (m, 1H), 2.66 (d, J=7.4 Hz, 2H), 2.60 (t, J=5.1 Hz, 4H), 2.34 (qd, J=13.2, 4.8 Hz, 1H), 2.25-2.17 (m, 1H), 1.46 (s, 9H). LC/MS (ESI) m/z 498.2; [M+H]⁺ calcd for C₂₆H₃₆N₅O₅⁺: 498.27.

3-(5-(4-(azetidin-3-ylmethyl) piperazin-1-yl)-1-oxoisoindolin-2-yl) piperidine-2,6-dione hydrochloride (24-2) (20 mg, quantitative yield). LC/MS (ESI) m/z 398.2; [M+H]⁺ calcd for C₂₁H₂₈N₅O₃⁺: 398.22.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((3-((4-(2-(2,6-dioxopiperidin-3-yl)-1-oxoisoindolin-5-yl) piperazin-1-yl)methyl) azetidin-1-yl)methyl)-4-methyl-3,4,5,6-tetrahydro-[1,1′-biphenyl]-2-yl)methyl) piperazin-1-yl)benzamide (compound #24) (4.23 mg, 12% yield). ¹H NMR (600 MHZ, Chloroform-d) & 8.32 (s, 1H), 7.98 (d, J=9.0 Hz, 1H), 7.91 (d, J=8.2 Hz, 2H), 7.66 (d, J=8.4 Hz, 1H), 7.38-7.30 (m, 2H), 7.28-7.22 (m, 4H), 7.19 (t, J=7.3 Hz, 1H), 6.96 (d, J=8.0 Hz, 2H), 6.89-6.79 (m, 2H), 6.79-6.72 (m, 1H), 6.66 (d, J=8.3 Hz, 2H), 6.45 (d, J=9.2 Hz, 1H), 5.10 (dd, J=13.1 Hz, 1H), 4.41-4.00 (m, 3H), 3.85-3.60 (m, 6H), 3.25-2.92 (m, 12H), 2.92-2.36 (m, 17H), 2.36-1.95 (m, 13H), 1.93-1.75 (m, 2H), 1.74-1.52 (m, 2H), 1.52-1.37 (m, 1H), 1.05 (s, 3H). LC/MS (ESI) m/z 1383.7; [M+H]⁺ calcd for C₆₉H₈₃ClF₃N₁₀O₉S₃⁺: 1383.51.

### Example 19: Preparation of Compound #26

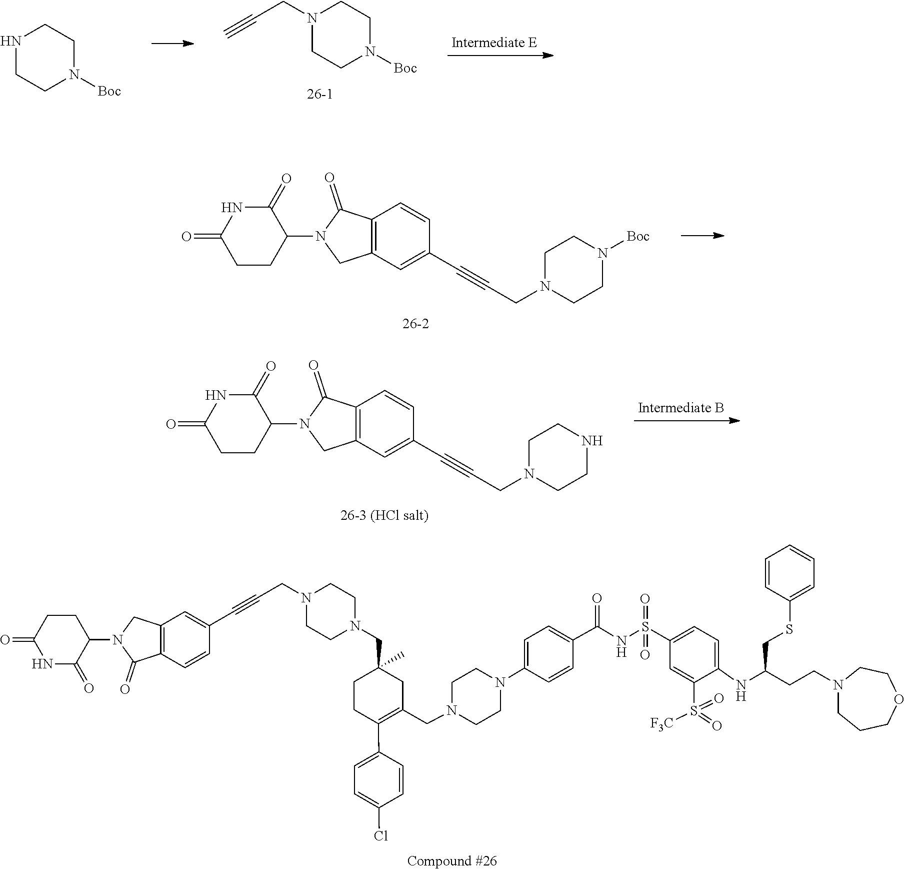 [SMILES: CC(C)(C)OC(=O)N1CCNCC1 ; C#CCN1CCN(C(=O)OC(C)(C)C)CC1 ; CC(C)(C)OC(=O)N1CCN(CC#Cc2ccc3c(c2)CN(C2CCC(=O)NC2=O)C3=O)CC1 ; O=C1CCC(N2Cc3cc(C#CCN4CCNCC4)ccc3C2=O)C(=O)N1 ; C[C@@]1(CN2CCN(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Step 1: Synthesis of tert-butyl 4-(prop-2-yn-1-yl) piperazine-1-carboxylate (26-1): A solution of N-Boc-piperazine (5 g, 26.88 mmol), 3-bromoprop-1-yne (6.4 g, 53.76 mmol) and DIPEA (18.7 mL, 107.53 mmol) in DCM (100 mL) was stirred at room temperature for 3 days. The reaction mixture was washed with water and sat. aq. NH₄Cl. The organic layers were dried over Na₂SO₄, filtered and concentrated. The residue was purified by flash column chromatography (20% to 100% of EtOAc in hexanes) to afford 26-1 (3.2 g, 53% yield) as light-yellow oil. 1H NMR (600 MHZ, Chloroform-d) δ 3.49 (t, J=5.1 Hz, 4H), 3.34 (d, J=2.5 Hz, 2H), 2.53 (t, J=5.1 Hz, 4H), 2.28 (t, J=2.4 Hz, 1H), 1.48 (s, 9H). LC/MS (ESI) m/z 225.2; [M+H]⁺ calcd for C₁₂H₂₁N₂O₂+: 225.16.

Steps 2-4 were Performed According to General Procedure B:

tert-butyl 4-(3-(2-(2,6-dioxopiperidin-3-yl)-1-oxoisoindolin-5-yl) prop-2-yn-1-yl) piperazine-1-carboxylate (26-2) (257 mg, 45% yield). ¹H NMR (600 MHZ, Chloroform-d) δ 8.07 (s, 1H), 7.85 (d, J=7.8 Hz, 1H), 7.57 (dd, J=7.8, 1.3 Hz, 1H), 7.54 (s, 1H), 5.24 (dd, J=13.3, 5.1 Hz, 1H), 4.50 (d, J=16.0 Hz, 1H), 4.35 (d, J=15.9 Hz, 1H), 3.59 (s, 2H), 3.53 (t, J=5.0 Hz, 4H), 3.00-2.91 (m, 1H), 2.86 (ddd, J=18.0, 13.4, 5.4 Hz, 1H), 2.61 (t, J=5.0 Hz, 4H), 2.38 (qd, J=13.3, 4.7 Hz, 1H), 2.31-2.19 (m, 1H), 1.49 (s, 9H). LC/MS (ESI) m/z 467.3; [M+H]⁺ calcd for C₂₅H₃₁N₄O₅⁺: 467.23.

3-(1-oxo-5-(3-(piperazin-1-yl) prop-1-yn-1-yl) isoindolin-2-yl) piperidine-2,6-dione hydrochloride (26-3) (63 mg, quantitative yield). LC/MS (ESI) m/z 367.1; [M+H]⁺ calcd for C₂₀H₂₃N₄O₃⁺: 367.18.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(3-(2-(2,6-dioxopiperidin-3-yl)-1-oxoisoindolin-5-yl) prop-2-yn-1-yl) piperazin-1-yl)methyl)-4-methyl-3,4,5,6-tetrahydro-[1,1′-biphenyl]-2-yl)methyl) piperazin-1-yl)benzamide (compound #26) (5.69 mg, 21% yield). ¹H NMR (600 MHZ, Chloroform-d) δ 8.32 (t, J=2.2 Hz, 1H), 8.14-8.07 (m, 1H), 7.76-7.70 (m, 2H), 7.53-7.47 (m, 2H), 7.41-7.36 (m, 2H), 7.34-7.22 (m, 6H), 7.14 (t, J=8.7 Hz, 1H), 7.02 (t, J=8.2 Hz, 2H), 6.68 (d, J=8.7 Hz, 1H), 6.64-6.56 (m, 2H), 5.42 (dt, J=13.6, 4.5 Hz, 1H), 4.47 (t, J=15.8 Hz, 1H), 4.36 (dd, J=16.3, 9.3 Hz, 1H), 3.92 (s, 1H), 3.77 (td, J=6.1, 1.1 Hz, 2H), 3.75-3.61 (m, 4H), 3.22 (dt, J=18.7, 4.3 Hz, 4H), 3.11 (dd, J=13.9, 5.1 Hz, 1H), 3.05 (dd, J=13.9, 6.9 Hz, 1H), 2.99-2.89 (m, 2H), 2.84 (s, 2H), 2.79-2.54 (m, 14H), 2.46-2.20 (m, 12H), 2.14-2.04 (m, 1H), 2.02-1.84 (m, 3H), 1.78-1.58 (m, 2H), 0.96 (d, J=13.3 Hz, 3H). LC/MS (ESI) m/z 1352.4; [M+H]⁺ calcd for C₆₈H₇₈ClF₃N₉₀₉S₃⁺: 1352.47.

### Example 20: Preparation of Compound #29

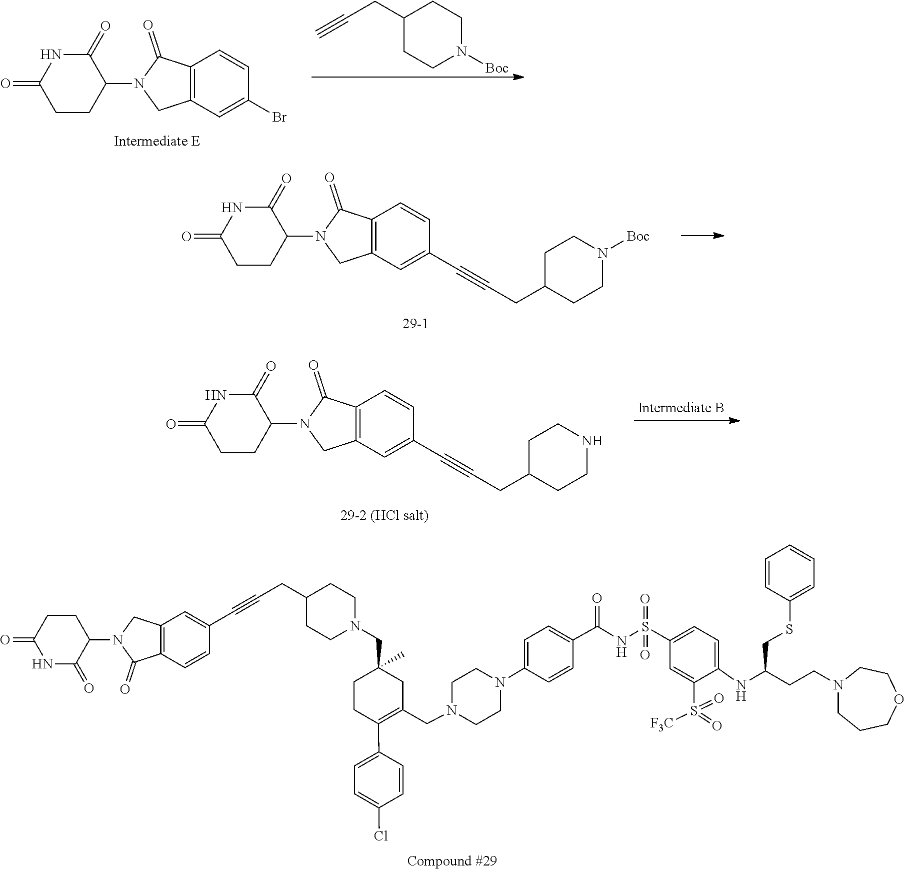 [SMILES: O=C1CCC(N2Cc3cc(Br)ccc3C2=O)C(=O)N1 ; C#CCC1CCN(C(=O)OC(C)(C)C)CC1 ; CC(C)(C)OC(=O)N1CCC(CC#Cc2ccc3c(c2)CN(C2CCC(=O)NC2=O)C3=O)CC1 ; O=C1CCC(N2Cc3cc(C#CCC4CCNCC4)ccc3C2=O)C(=O)N1 ; C[C@@]1(CN2CCC(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #29 was Prepared by Following General Procedure B:

2 tert-butyl 4-(3-(2-(2,6-dioxopiperidin-3-yl)-1-oxoisoindolin-5-yl) prop-2-yn-1-yl) piperidine-1-carboxylate (29-1) (487 mg, 68% yield). ¹H NMR (600 MHZ, Chloroform-d) δ 8.02 (s, 1H), 7.82 (d, J=7.8 Hz, 1H), 7.52 (dd, J=7.8, 1.3 Hz, 1H), 7.50 (s, 1H), 5.23 (dd, J=13.4, 5.1 Hz, 1H), 4.49 (d, J=15.9 Hz, 1H), 4.33 (d, J=16.0 Hz, 1H), 4.17 (s, 2H), 2.99-2.91 (m, 1H), 2.86 (ddd, J=18.1, 13.4, 5.4 Hz, 1H), 2.75 (s, 2H), 2.43 (d, J=6.6 Hz, 2H), 2.41-2.32 (m, 1H), 2.29-2.21 (m, 1H), 1.84 (d, J=13.0 Hz, 2H), 1.80-1.71 (m, 1H), 1.48 (s, 9H), 1.30 (qd, J=12.3, 4.3 Hz, 2H). LC/MS (ESI) m/z 488.2 (M+23), 410.1 (M-56), 366.2 (M-Boc); [M+H]⁺ calcd for C₂₆H₃₂N₃O₅⁺: 466.23.

3-(1-oxo-5-(3-(piperidin-4-yl) prop-1-yn-1-yl) isoindolin-2-yl) piperidine-2,6-dione hydrochloride (29-2) (210 mg, quantitative yield). LC/MS (ESI) m/z 366.3; [M+H]⁺ calcd for C₂₁H₂₄N₃O₃⁺: 366.44.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(3-(2-(2,6-dioxopiperidin-3-yl)-1-oxoisoindolin-5-yl) prop-2-yn-1-yl) piperidin-1-yl)methyl)-4-methyl-3,4,5,6-tetrahydro-[1,1′-biphenyl]-2-yl)methyl) piperazin-1-yl)benzamide (compound #29) (6.02 mg, 22% yield). ¹H NMR (600 MHZ, Chloroform-d) δ 8.34 (d, J=2.2 Hz, 1H), 8.11-8.01 (m, 1H), 7.77 (t, J=8.3 Hz, 2H), 7.66 (dd, J=34.5, 7.8 Hz, 1H), 7.45 (d, J=9.6 Hz, 2H), 7.41-7.35 (m, 2H), 7.35-7.21 (m, 6H), 7.08-6.96 (m, 3H), 6.67 (s, 1H), 6.61 (d, J=8.5 Hz, 1H), 6.53 (d, J=9.2 Hz, 1H), 5.34-5.23 (m, 1H), 4.41 (t, J=17.1 Hz, 1H), 4.29 (d, J=16.0 Hz, 1H), 3.87 (s, 1H), 3.81-3.77 (m, 2H), 3.75-3.68 (m, 2H), 3.20 (s, 4H), 3.11 (dd, J=13.9, 4.9 Hz, 1H), 3.04 (dd, J=13.9, 6.9 Hz, 1H), 2.98-2.57 (m, 12H), 2.52-2.15 (m, 16H), 2.16-2.06 (m, 1H), 2.07-1.85 (m, 3H), 1.83-1.55 (m, 6H), 1.49-1.38 (m, 1H), 1.00 (d, J=15.0 Hz, 3H). LC/MS (ESI) m/z 1351.6; [M+H]⁺ calcd for C₆₉H₇₉ClF₃N₈O₉S₃⁺: 1351.48.

### Example 21: Preparation of Compounds #37-38

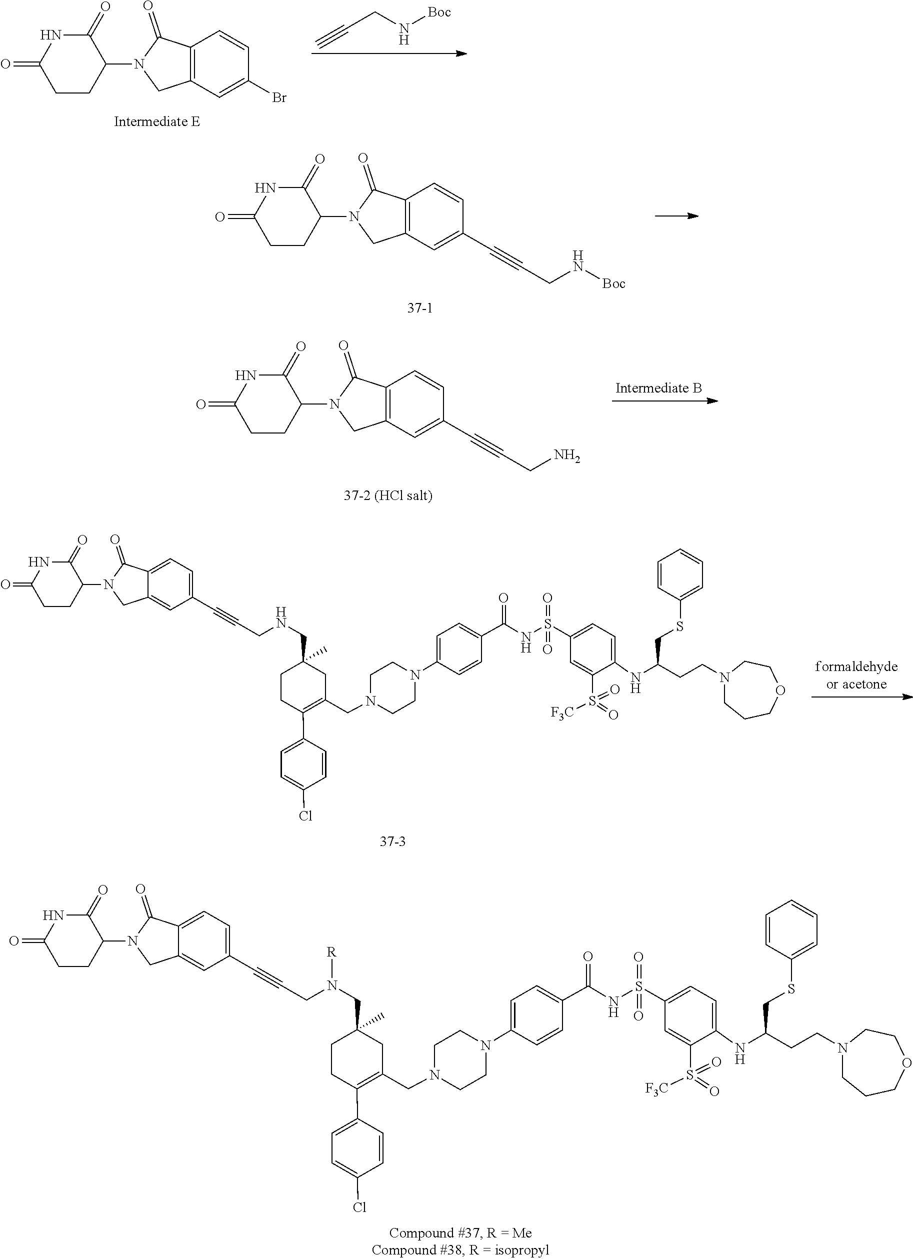

Compounds #37-38 were prepared by following General Procedure B: tert-butyl (3-(2-(2,6-dioxopiperidin-3-yl)-1-oxoisoindolin-5-yl) prop-2-yn-1-yl) carbamate (37-1) (364 mg, 59% yield). ¹H NMR (600 MHZ, Chloroform-d) δ 8.05 (s, 1H), 7.84 (d, J=7.9 Hz, 1H), 7.55 (dd, J=8.0, 1.3 Hz, 1H), 7.53 (s, 1H), 5.24 (dd, J=13.4, 5.1 Hz, 1H), 4.83 (s, 1H), 4.50 (d, J=16.0 Hz, 1H), 4.34 (d, J=16.0 Hz, 1H), 4.21 (s, 2H), 3.00-2.91 (m, 1H), 2.86 (ddd, J=18.1, 13.4, 5.4 Hz, 1H), 2.38 (qd, J=13.3, 4.7 Hz, 1H), 2.29-2.20 (m, 1H), 1.50 (s, 9H). LC/MS (ESI) m/z 398.1; [M+H]⁺ calcd for C₂₁H₂₄N₃O₅⁺: 398.17.

3-(5-(3-aminoprop-1-yn-1-yl)-1-oxoisoindolin-2-yl) piperidine-2,6-dione hydrochloride (37-2) (106 mg, quantitative yield). LC/MS (ESI) m/z 298.1; [M+H]⁺ calcd for C₁₆H₁₆N₃O₃⁺: 298.12.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-(((3-(2-(2,6-dioxopiperidin-3-yl)-1-oxoisoindolin-5-yl) prop-2-yn-1-yl)amino)methyl)-4-methyl-3,4,5,6-tetrahydro-[1,1′-biphenyl]-2-yl)methyl) piperazin-1-yl)benzamide (37-3) (50 mg, 78% yield). LC/MS (ESI) m/z 1283.5; [M+H]⁺ calcd for C₆₄H₇₁ClF₃N₈O₉S₃⁺: 1283.41.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-(((3-(2-(2,6-dioxopiperidin-3-yl)-1-oxoisoindolin-5-yl) prop-2-yn-1-yl)(methyl)amino)methyl)-4-methyl-3,4,5,6-tetrahydro-[1,1′-biphenyl]-2-yl)methyl) piperazin-1-yl)benzamide (compound #37) (7.39 mg, 49% yield). ¹H NMR (600 MHZ, Chloroform-d) δ 8.33 (d, J=2.2 Hz, 1H), 8.16-8.07 (m, 1H), 7.78-7.60 (m, 3H), 7.56-7.49 (m, 2H), 7.38 (d, J=7.2 Hz, 2H), 7.34-7.21 (m, 6H), 7.12 (t, J=10.2 Hz, 1H), 7.07-7.02 (m, 1H), 7.02-6.99 (m, 1H), 6.62 (d, J=9.2 Hz, 1H), 6.48 (d, J=8.6 Hz, 1H), 6.44 (d, J=8.7 Hz, 1H), 5.35 (d, J=13.1 Hz, 1H), 4.60-4.24 (m, 2H), 3.92 (s, 1H), 3.77 (td, J=6.1, 1.4 Hz, 2H), 3.74-3.66 (m, 2H), 3.64 (t, J=5.9 Hz, 2H), 3.16-2.97 (m, 6H), 2.96-2.87 (m, 2H), 2.87-2.54 (m, 9H), 2.52 (d, J=3.2 Hz, 3H), 2.47-2.20 (m, 8 H), 2.12-2.03 (m, 1H), 1.99-1.85 (m, 3H), 1.85-1.77 (m, 1H), 1.76-1.69 (m, 1H), 1.54-1.42 (m, 1H), 1.06 (s, 3H). LC/MS (ESI) m/z 1297.5; [M+H]⁺ calcd for C₆₅H₇₃ClF₃N₈O₉S₃⁺: 1297.43.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-(((3-(2-(2,6-dioxopiperidin-3-yl)-1-oxoisoindolin-5-yl) prop-2-yn-1-yl) (isopropyl)amino)methyl)-4-methyl-3,4,5,6-tetrahydro-[1,1′-biphenyl]-2-yl)methyl) piperazin-1-yl)benzamide (compound #38) (2.8 mg, 18% yield). ¹H NMR (600 MHZ, Chloroform-d) δ 8.33 (dd, J=8.7, 2.3 Hz, 1H), 8.11 (dd, J=15.0, 9.2 Hz, 1H), 7.70 (td, J=27.6, 8.2 Hz, 2H), 7.60-7.43 (m, 2H), 7.39 (d, J=7.6 Hz, 2H), 7.35-7.23 (m, 6H), 7.21-7.12 (m, 1H), 7.09-7.04 (m, 1H), 7.04-6.99 (m, 1H), 6.62 (d, J=9.3 Hz, 1H), 6.49 (d, J=8.6 Hz, 1H), 6.38 (d, J=8.6 Hz, 1H), 5.33 (dd, J=52.2, 13.5 Hz, 1H), 4.57-4.24 (m, 2H), 3.92 (s, 1H), 3.82-3.73 (m, 2H), 3.74-3.63 (m, 4H), 3.18-3.00 (m, 6H), 2.99-2.89 (m, 2H), 2.89-2.77 (m, 1H), 2.76-2.51 (m, 8H), 2.51-2.17 (m, 11H), 2.13-2.03 (m, 1H), 2.01-1.93 (m, 1H), 1.90-1.62 (m, 4H), 1.46-1.38 (m, 1H), 1.15 (dd, J=6.6, 2.0 Hz, 6H), 1.01 (d, J=11.6 Hz, 3H). LC/MS (ESI) m/z 1325.6; [M+H]⁺ calcd for C₆₇H₇₇ClF₃N₈O₉S₃⁺: 1325.46.

### Example 22: Preparation of Compound #40

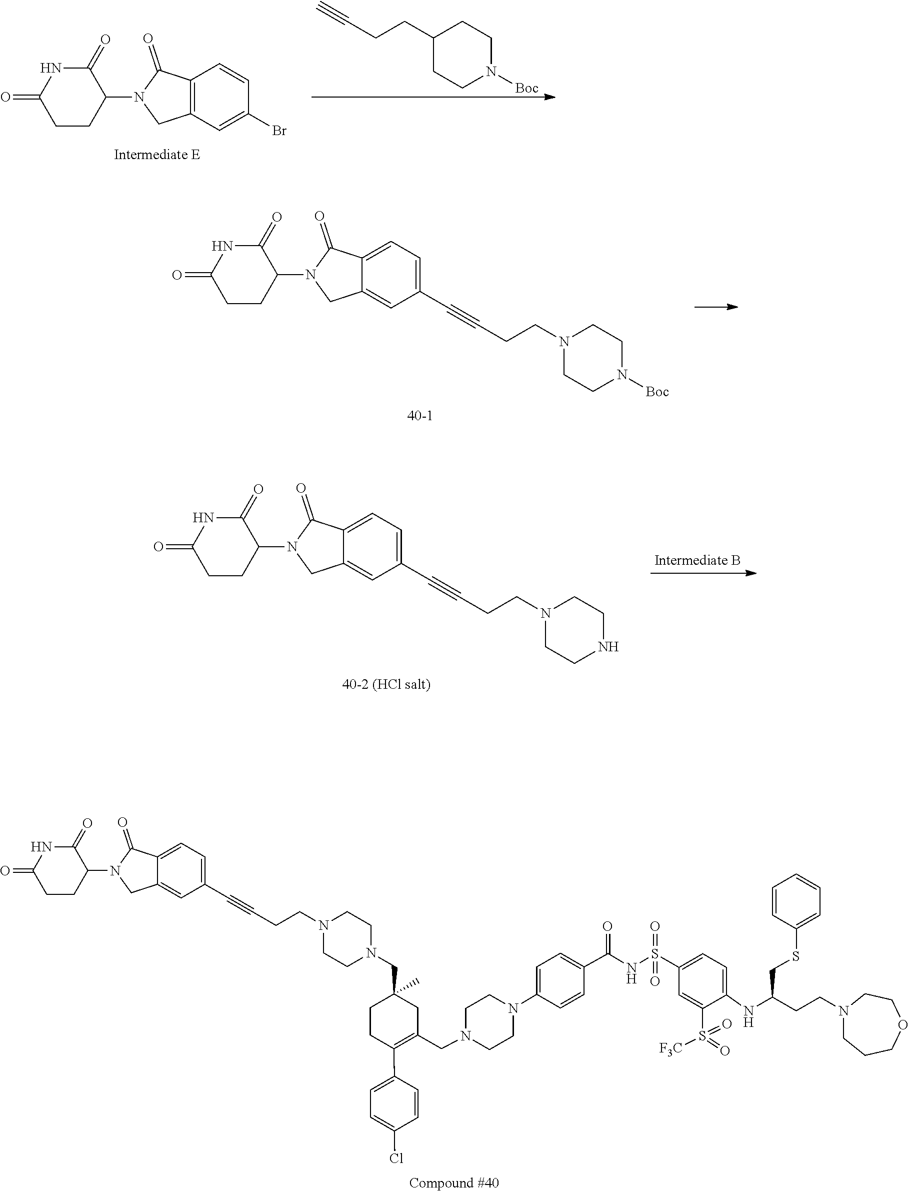 [SMILES: O=C1CCC(N2Cc3cc(Br)ccc3C2=O)C(=O)N1 ; C#CCCC1CCN(C(=O)OC(C)(C)C)CC1 ; CC(C)(C)OC(=O)N1CCN(CCC#Cc2ccc3c(c2)CN(C2CCC(=O)NC2=O)C3=O)CC1 ; O=C1CCC(N2Cc3cc(C#CCCN4CCNCC4)ccc3C2=O)C(=O)N1 ; C[C@@]1(CN2CCN(CCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #40 was Prepared by Following General Procedure B:

tert-butyl 4-(4-(2-(2,6-dioxopiperidin-3-yl)-1-oxoisoindolin-5-yl) but-3-yn-1-yl) piperazine-1-carboxylate (40-1) (280 mg, 38% yield). ¹H NMR (600 MHZ, Chloroform-d) δ 8.08 (s, 1H), 7.82 (d, J=7.8 Hz, 1H), 7.52 (dd, J=8.0, 1.3 Hz, 1H), 7.49 (s, 1H), 5.23 (dd, J=13.3, 5.1 Hz, 1H), 4.49 (d, J=16.0 Hz, 1H), 4.33 (d, J=16.0 Hz, 1H), 3.48 (t, J=5.1 Hz, 4H), 2.95 (ddd, J=17.8, 4.7, 2.5 Hz, 1H), 2.85 (ddd, J=18.1, 13.4, 5.4 Hz, 1H), 2.76-2.70 (m, 2H), 2.68-2.62 (m, 2H), 2.51 (t, J=5.1 Hz, 4H), 2.37 (qd, J=13.3, 4.7 Hz, 1H), 2.28-2.20 (m, 1H), 1.49 (s, 9H). LC/MS (ESI) m/z 481.2; [M+H]⁺ calcd for C₂₆H₃₃N₄O₅⁺: 481.24.

3-(1-oxo-5-(4-(piperazin-1-yl) but-1-yn-1-yl) isoindolin-2-yl) piperidine-2,6-dione hydrochloride (40-2) (121 mg, quantitative yield). LC/MS (ESI) m/z 381.1; [M+H]⁺ calcd for C₂₁H₂₅N₄O₃⁺: 381.19.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(4-(2-(2,6-dioxopiperidin-3-yl)-1-oxoisoindolin-5-yl) but-3-yn-1-yl) piperazin-1-yl)methyl)-4-methyl-3,4,5,6-tetrahydro-[1,1′-biphenyl]-2-yl)methyl) piperazin-1-yl)benzamide (compound #40) (4.93 mg, 24% yield). ¹H NMR (600 MHZ, Chloroform-d) δ 8.34 (t, J=2.7 Hz, 1H), 8.06 (dt, J=9.4, 2.9 Hz, 1H), 7.78 (dd, J=9.1, 2.9 Hz, 2H), 7.70 (dd, J=23.5, 8.1 Hz, 1H), 7.48-7.42 (m, 2H), 7.41-7.35 (m, 2H), 7.34-7.22 (m, 5H), 7.07 (d, J=8.5 Hz, 1H), 7.03-6.98 (m, 2H), 6.74 (dd, J=13.7, 8.8 Hz, 2H), 6.57 (d, J=9.4 Hz, 1H), 5.32-5.24 (m, 1H), 4.45 (dd, J=16.2, 6.4 Hz, 1H), 4.32 (d, J=16.2 Hz, 1H), 3.90 (s, 1H), 3.78 (t, J=6.1 Hz, 2H), 3.75-3.69 (m, 1H), 3.23 (s, 4H), 3.11 (dd, J=14.0, 5.0 Hz, 1H), 3.04 (dd, J=13.9, 7.0 Hz, 1H), 2.97-2.54 (m, 23H), 2.50-2.19 (m, 10H), 2.15-2.05 (m, 1H), 1.97-1.87 (m, 3H), 1.80-1.70 (m, 1H), 1.69-1.56 (m, 2H), 1.48-1.39 (m, 1H), 0.97 (d, J=3.2 Hz, 3H). LC/MS (ESI) m/z 1366.6; [M+H]⁺ calcd for C₆₉H₈₀ClF₃N₉₀₉S₃⁺: 1366.49.

### Example 23: Preparation of Compound #44

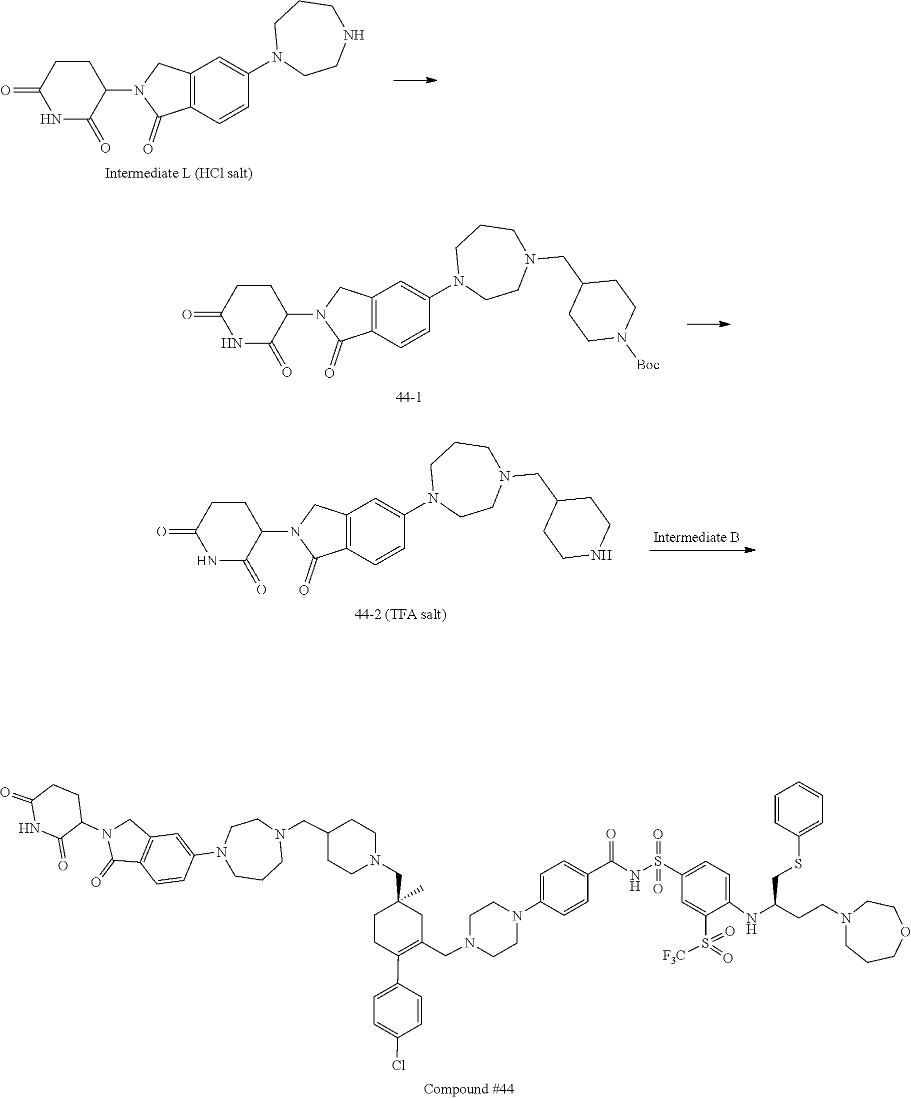 [SMILES: O=C1CCC(N2Cc3cc(N4CCCNCC4)ccc3C2=O)C(=O)N1 ; CC(C)(C)OC(=O)N1CCC(CN2CCCN(c3ccc4c(c3)CN(C3CCC(=O)NC3=O)C4=O)CC2)CC1 ; O=C1CCC(N2Cc3cc(N4CCCN(CC5CCNCC5)CC4)ccc3C2=O)C(=O)N1 ; C[C@@]1(CN2CCC(CN3CCCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #44 was Prepared by Following General Procedure C:

tert-butyl 4-((4-(2-(2,6-dioxopiperidin-3-yl)-1-oxoisoindolin-5-yl)-1,4-diazepan-1-yl)methyl) piperidine-1-carboxylate (44-1) (58 mg, 79% yield). ¹H NMR (600 MHZ, Chloroform-d) δ 7.90 (s, 1H), 7.68 (d, J=8.6 Hz, 1H), 6.75 (dd, J=8.7, 2.3 Hz, 1H), 6.64 (d, J=2.2 Hz, 1H), 5.20 (dd, J=13.3, 5.1 Hz, 1H), 4.38 (d, J=15.4 Hz, 1H), 4.23 (d, J=15.4 Hz, 1H), 4.07 (s, 2H), 3.61-3.52 (m, 4H), 2.93-2.87 (m, 1H), 2.82 (ddd, J=18.0, 13.3, 5.4 Hz, 1H), 2.77 (t, J=4.9 Hz, 2H), 2.72-2.62 (m, 2H), 2.61-2.54 (m, 2H), 2.35-2.26 (m, 3H), 2.24-2.15 (m, 1H), 1.94 (p, J=5.9 Hz, 2H), 1.77-1.51 (m, 3H), 1.45 (s, 9H), 1.04 (q, J=12.1 Hz, 2H). LC/MS (ESI) m/z 540.2; [M+H]⁺ calcd for C₂₉H₄₂N₅O₅⁺: 540.32.

3-(1-oxo-5-(4-(piperidin-4-ylmethyl)-1,4-diazepan-1-yl) isoindolin-2-yl) piperidine-2,6-dione TFA salt (44-2) (31 mg, quantitative yield). LC/MS (ESI) m/z 440.2; [M+H]⁺ calcd for C₂₄H₃₄N₅O₃⁺: 440.27.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-(2-(2,6-dioxopiperidin-3-yl)-1-oxoisoindolin-5-yl)-1,4-diazepan-1-yl)methyl) piperidin-1-yl)methyl)-4-methyl-3,4,5,6-tetrahydro-[1,1′-biphenyl]-2-yl)methyl) piperazin-1-yl)benzamide (compound #44) (9.3 mg, 26% yield). ¹H NMR (600 MHZ, Chloroform-d) δ 8.31 (s, 1H), 7.99 (d, J=8.9 Hz, 1H), 7.87 (d, J=8.3 Hz, 2H), 7.63 (d, J=8.5 Hz, 1H), 7.34 (d, J=7.6 Hz, 2H), 7.31-7.14 (m, 5H), 6.96 (d, J=8.0 Hz, 2H), 6.89 (d, J=8.3 Hz, 1H), 6.77-6.65 (m, 3H), 6.62 (s, 1H), 6.44 (d, J=9.1 Hz, 1H), 5.13 (d, J=13.0 Hz, 1H), 4.33 (d, J=15.8 Hz, 1H), 4.20 (d, J=15.7 Hz, 1H), 3.87-3.61 (m, 5H), 3.61-3.42 (m, 4H), 3.24-2.93 (m, 7H), 2.89-2.51 (m, 16H), 2.51-2.16 (m, 16H), 2.15-1.97 (m, 2H), 1.95-1.78 (m, 5H), 1.77-1.54 (m, 4H), 1.50-1.39 (m, 1H), 0.99 (s, 3H). LC/MS (ESI) m/z 1425.7; [M+H]⁺ calcd for C₇₂H₈₉ClF₃N₁₀O₉S₃⁺: 1425.56.

### Example 24: Preparation of Compound #45

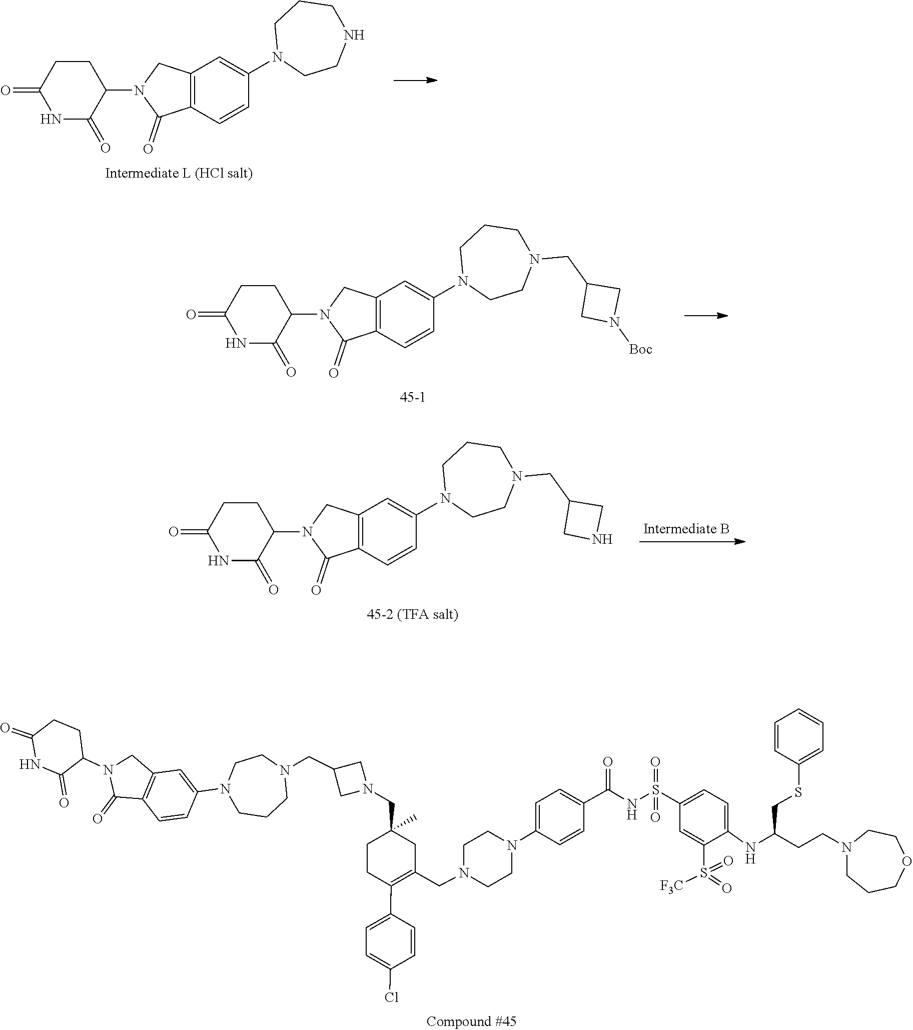 [SMILES: O=C1CCC(N2Cc3cc(N4CCCNCC4)ccc3C2=O)C(=O)N1 ; CC(C)(C)OC(=O)N1CC(CN2CCCN(c3ccc4c(c3)CN(C3CCC(=O)NC3=O)C4=O)CC2)C1 ; O=C1CCC(N2Cc3cc(N4CCCN(CC5CNC5)CC4)ccc3C2=O)C(=O)N1 ; C[C@@]1(CN2CC(CN3CCCN(c4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Compound #45 was prepared by following General Procedure C: tert-butyl 3-((4-(2-(2,6-dioxopiperidin-3-yl)-1-oxoisoindolin-5-yl)-1,4-diazepan-1-yl)methyl) azetidine-1-carboxylate (45-1) (47 mg, 68% yield). ¹H NMR (600 MHZ, Chloroform-d) δ 7.95 (s, 1H), 7.68 (d, J=8.6 Hz, 1H), 6.75 (dd, J=8.7, 2.3 Hz, 1H), 6.63 (d, J=2.2 Hz, 1H), 5.19 (dd, J=13.4, 5.1 Hz, 1H), 4.38 (d, J=15.4 Hz, 1H), 4.24 (d, J=15.4 Hz, 1H), 3.97 (td, J=8.2, 1.6 Hz, 2H), 3.61-3.51 (m, 6H), 2.90 (ddd, J=17.5, 5.7, 2.9 Hz, 1H), 2.82 (ddd, J=17.9, 13.3, 5.4 Hz, 1H), 2.76 (t, J=4.9 Hz, 2H), 2.72-2.62 (m, 3H), 2.61-2.55 (m, 2H), 2.31 (qd, J=13.3, 4.8 Hz, 1H), 2.22-2.15 (m, 1H), 1.95 (p, J=6.1 Hz, 2H), 1.43 (s, 9H).

LC/MS (ESI) m/z 512.4; [M+H]⁺ calcd for C₂₇H₃₈N₅O₅⁺: 512.29.

3-(5-(4-(azetidin-3-ylmethyl)-1,4-diazepan-1-yl)-1-oxoisoindolin-2-yl) piperidine-2,6-dione TFA salt (45-2) (28 mg, quantitative yield). LC/MS (ESI) m/z 412.3; [M+H]⁺ calcd for C₂₂H₃₀N₅O₃⁺: 412.23.

l N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((3-((4-(2-(2,6-dioxopiperidin-3-yl)-1-oxoisoindolin-5-yl)-1,4-diazepan-1-yl)methyl) azetidin-1-yl)methyl)-4-methyl-3,4,5,6-tetrahydro-[1,1′-biphenyl]-2-yl)methyl) piperazin-1-yl)benzamide (compound #45) (6.3 mg, 18% yield). ¹H NMR (600 MHZ, Chloroform-d) δ 8.32 (s, 1H), 7.98 (d, J=9.0 Hz, 1H), 7.92 (d, J=8.3 Hz, 2H), 7.63 (d, J=8.5 Hz, 1H), 7.35 (d, J=7.6 Hz, 2H), 7.30-7.16 (m, 5H), 6.95 (d, J=8.0 Hz, 2H), 6.87 (d, J=8.3 Hz, 1H), 6.73-6.62 (m, 3H), 6.59 (s, 1H), 6.46 (d, J=9.2 Hz, 1H), 5.10 (s, 1H), 4.32 (d, J=15.9 Hz, 1H), 4.19 (d, J=15.6 Hz, 1H), 3.81 (s, 1H), 3.73 (t, J=6.1 Hz, 2H), 3.71-3.60 (m, 2H), 3.57-3.35 (m, 5H), 3.15-2.87 (m, 8H), 2.86-2.45 (m, 20H), 2.35-2.13 (m, 8H), 2.13-1.95 (m, 3H), 1.93-1.75 (m, 5H), 1.74-1.48 (m, 2H), 1.48-1.37 (m, 1H), 1.01 (s, 3H). LC/MS (ESI) m/z 1397.6; [M+H]⁺ calcd for C₇₀H₈₅ClF₃N₁₀O₉S₃⁺: 1397.53.

### Example 25: Preparation of Compounds #46

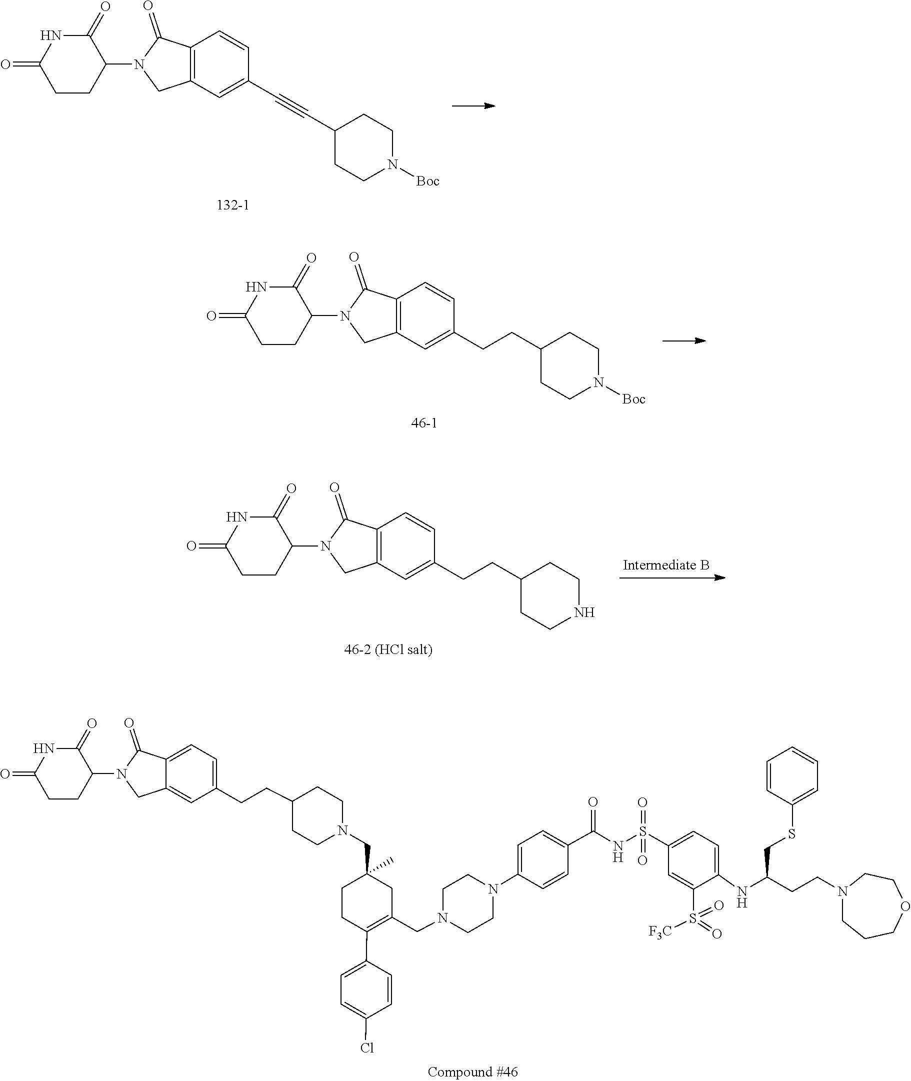 [SMILES: CC(C)(C)OC(=O)N1CCC(C#Cc2ccc3c(c2)CN(C2CCC(=O)NC2=O)C3=O)CC1 ; CC(C)(C)OC(=O)N1CCC(CCc2ccc3c(c2)CN(C2CCC(=O)NC2=O)C3=O)CC1 ; O=C1CCC(N2Cc3cc(CCC4CCNCC4)ccc3C2=O)C(=O)N1 ; C[C@@]1(CN2CCC(CCc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #46 was Prepared by Following General Procedure E:

tert-butyl 4-(2-(2-(2,6-dioxopiperidin-3-yl)-1-oxoisoindolin-5-yl)ethyl) piperidine-1-carboxylate (46-1) (170 mg, 56% yield). ¹H NMR (600 MHZ, Chloroform-d) δ 8.02 (s, 1H), 7.82 (d, J=7.8 Hz, 1H), 7.32 (dd, J=7.9, 1.4 Hz, 1H), 7.29 (s, 1H), 5.25 (dd, J=13.3, 5.1 Hz, 1H), 4.49 (d, J=15.8 Hz, 1H), 4.33 (d, J=15.8 Hz, 1H), 4.20-4.01 (m, 2H), 2.94 (ddd, J=17.8, 4.7, 2.5 Hz, 1H), 2.86 (ddd, J=18.0, 13.4, 5.4 Hz, 1H), 2.80-2.73 (m, 2H), 2.73-2.63 (m, 2H), 2.38 (qd, J=13.2, 4.8 Hz, 1H), 2.29-2.19 (m, 1H), 1.73 (d, J=13.0 Hz, 2H), 1.66-1.57 (m, 2H), 1.50-1.40 (m, 10H), 1.17 (qd, J=12.5, 4.4 Hz, 2H). LC/MS (ESI) m/z 478.3 (M+23), 400.2 (M-56), 356.2 (M-Boc); [M+H]⁺ calcd for C₂₅H₃₄N₃O₅⁺: 456.25.

3-(1-oxo-5-(2-(piperidin-4-yl)ethyl) isoindolin-2-yl) piperidine-2,6-dione hydrochloride (46-2) (22 mg, quantitative yield). LC/MS (ESI) m/z 356.2; [M+H]⁺ calcd for C₂₀H₂₆N₃O₃⁺: 356.20. N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(2-(2,6-dioxopiperidin-3-yl)-1-oxoisoindolin-5-yl)ethyl) piperidin-1-yl)methyl)-4-methyl-3,4,5,6-tetrahydro-[1,1′-biphenyl]-2-yl)methyl) piperazin-1-yl)benzamide (compound #46) (7.14 mg, 27% yield). ¹H NMR (600 MHZ, Chloroform-d) δ 8.56 (s, 1H), 8.35 (d, J=2.2 Hz, 1H), 8.05-8.00 (m, 1H), 7.86 (dd, J=8.8, 2.7 Hz, 2H), 7.75 (t, J=7.6 Hz, 1H), 7.40-7.36 (m, 2H), 7.33-7.27 (m, 6H), 7.26-7.20 (m, 1H), 6.99 (dd, J=8.4, 2.2 Hz, 2H), 6.93 (d, J=8.5 Hz, 1H), 6.72 (d, J=8.1 Hz, 2H), 6.47 (d, J=9.3 Hz, 1H), 5.23 (dt, J=13.3, 4.6 Hz, 1H), 4.44 (dd, J=16.1, 3.1 Hz, 1H), 4.31 (d, J=16.0 Hz, 1H), 3.82 (s, 1H), 3.78 (t, J=6.2 Hz, 2H), 3.75-3.71 (m, 2H), 3.35-3.14 (m, 8H), 3.11 (dd, J=13.8, 4.8 Hz, 1H), 3.03 (dd, J=13.9, 6.9 Hz, 1H), 2.96-2.74 (m, 9H), 2.74-2.48 (m, 8H), 2.47-2.25 (m, 6H), 2.25-2.17 (m, 1H), 2.16-2.07 (m, 1H), 1.93 (s, 2H), 1.81-1.68 (m, 4H), 1.68-1.52 (m, 5H), 1.51-1.44 (m, 1H), 1.06 (s, 3H). LC/MS (ESI) m/z 1341.5; [M+H]⁺ calcd for C₆₈H₈₁ClF₃N₈O₉S₃⁺: 1341.49.

### Example 26: Preparation of Compound #50

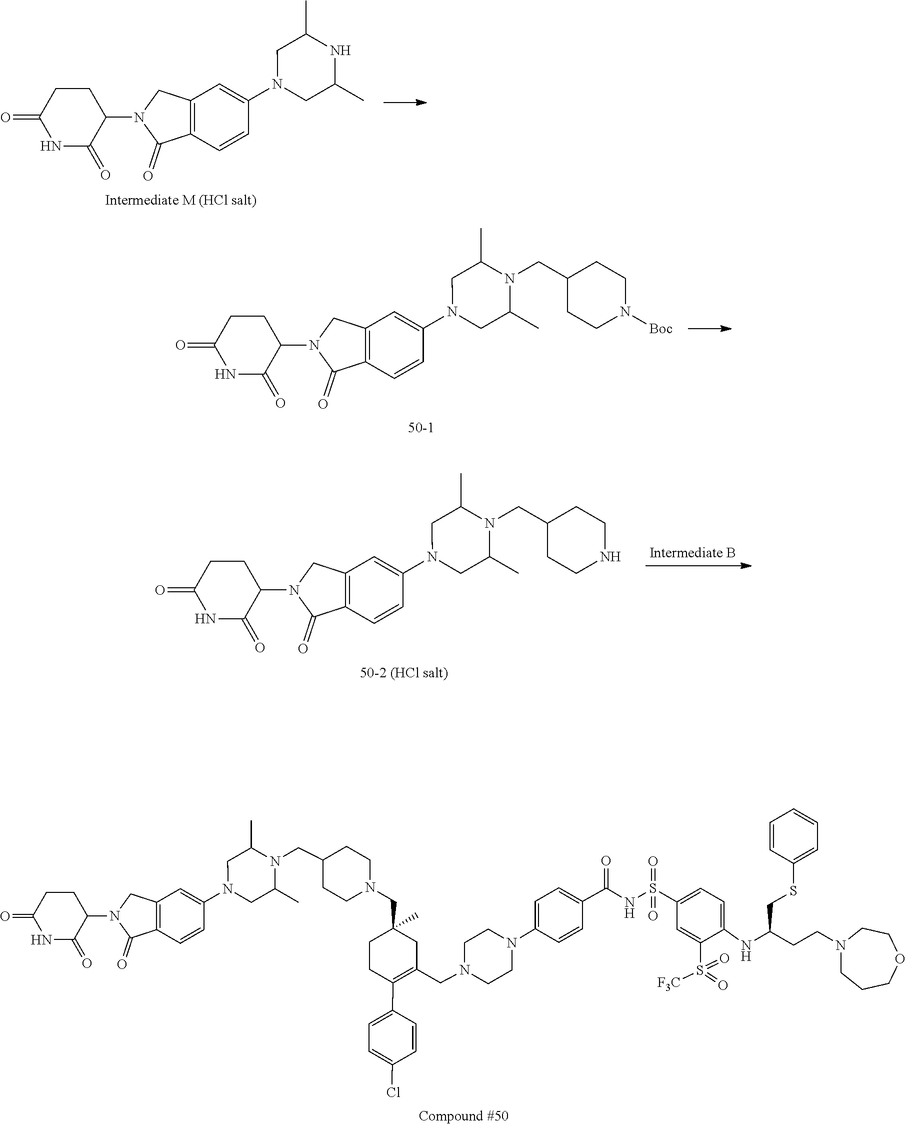 [SMILES: CC1CN(c2ccc3c(c2)CN(C2CCC(=O)NC2=O)C3=O)CC(C)N1 ; CC1CN(c2ccc3c(c2)CN(C2CCC(=O)NC2=O)C3=O)CC(C)N1CC1CCN(C(=O)OC(C)(C)C)CC1 ; CC1CN(c2ccc3c(c2)CN(C2CCC(=O)NC2=O)C3=O)CC(C)N1CC1CCNCC1 ; CC1CN(c2ccc3c(c2)CN(C2CCC(=O)NC2=O)C3=O)CC(C)N1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1]

### Compound #50 was Prepared by Following General Procedure C:

tert-butyl 4-((4-(2-(2,6-dioxopiperidin-3-yl)-1-oxoisoindolin-5-yl)-2,6-dimethylpiperazin-1-yl)methyl) piperidine-1-carboxylate (50-1) (17 mg, 70% yield). ¹H NMR (600 MHZ, 20 Chloroform-d) δ 8.37 (s, 1H), 7.74 (d, J=8.6 Hz, 1H), 6.97 (dd, J=8.6, 2.2 Hz, 1H), 6.85 (d, J=2.0 Hz, 1H), 5.24 (dd, J=13.3, 5.1 Hz, 1H), 4.42 (d, J=15.7 Hz, 1H), 4.27 (d, J=15.7 Hz, 1H), 2.95-2.81 (m, 4H), 2.81-2.62 (m, 6H), 2.53-2.40 (m, 4H), 2.39-2.28 (m, 1H), 2.25-2.17 (m, 1H), 1.85-1.77 (m, 2H), 1.70-1.59 (m, 1H), 1.48 (s, 9H), 1.18 (d, J=4.8 Hz, 6H), 1.13-1.03 (m, 2H). LC/MS (ESI) m/z 554.4; [M+H]⁺ calcd for C₃₀H₄₄N₅O₅⁺: 554.33.

3-(5-(3,5-dimethyl-4-(piperidin-4-ylmethyl) piperazin-1-yl)-1-oxoisoindolin-2-yl) piperidine-2,6-dione hydrochloride (50-2) (11 mg, quantitative yield). LC/MS (ESI) m/z 454.4; [M+H]⁺ calcd for C₂₅H₃₆N₅O₃⁺: 454.28.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-(2-(2,6-dioxopiperidin-3-yl)-1-oxoisoindolin-5-yl)-2,6-dimethylpiperazin-1-yl)methyl) piperidin-1-yl)methyl)-4-methyl-3,4,5,6-tetrahydro-[1,1′-biphenyl]-2-yl)methyl) piperazin-1-8 yl)benzamide (compound #50) (4.64 mg, 27% yield). ¹H NMR (600 MHZ, Chloroform-d) δ 8.34 (d, J=2.2 Hz, 1H), 8.16 (s, 1H), 8.03 (d, 1H), 7.78 (d, J=8.4 Hz, 2H), 7.70 (d, J=8.6 Hz, 1H), 7.36 (d, J=7.3 Hz, 2H), 7.32-7.19 (m, 5H), 6.98 (d, J=8.0 Hz, 3H), 6.92 (d, 1H), 6.81 (s, 1H), 6.72 (d, J=8.5 Hz, 2H), 6.48 (d, J=9.2 Hz, 1H), 5.18 (dd, J=13.3, 5.1 Hz, 1H), 4.39 (d, J=15.2 Hz, 1H), 4.24 (d, J=15.7 Hz, 1H), 3.83 (s, 1H), 3.76 (t, J=6.1 Hz, 2H), 3.73-3.62 (m, 2H), 3.54-3.44 (m, 2H), 3.20 (s, 4H), 3.09 (dd, J=13.8, 4.9 Hz, 1H), 3.01 (dd, J=13.9, 6.9 Hz, 1H), 2.94-2.54 (m, 14H), 2.51-2.23 (m, 12H), 2.22-2.15 (m, 1H), 2.14-2.05 (m, 2H), 2.03-1.97 (m, 1H), 1.96-1.84 (m, 2H), 1.84-1.67 (m, 3H), 1.67-1.58 (m, 3H), 1.53-1.39 (m, 3H), 1.37-1.28 (m, 2H), 1.10 (s, 6H), 1.03 (s, 3H). LC/MS (ESI) m/z 1439.7; [M+H]⁺ calcd for C₇₃H₉₁ClF₃N₁₀O₉S₃⁺: 1439.58.

### Example 27: Preparation of Compound #65

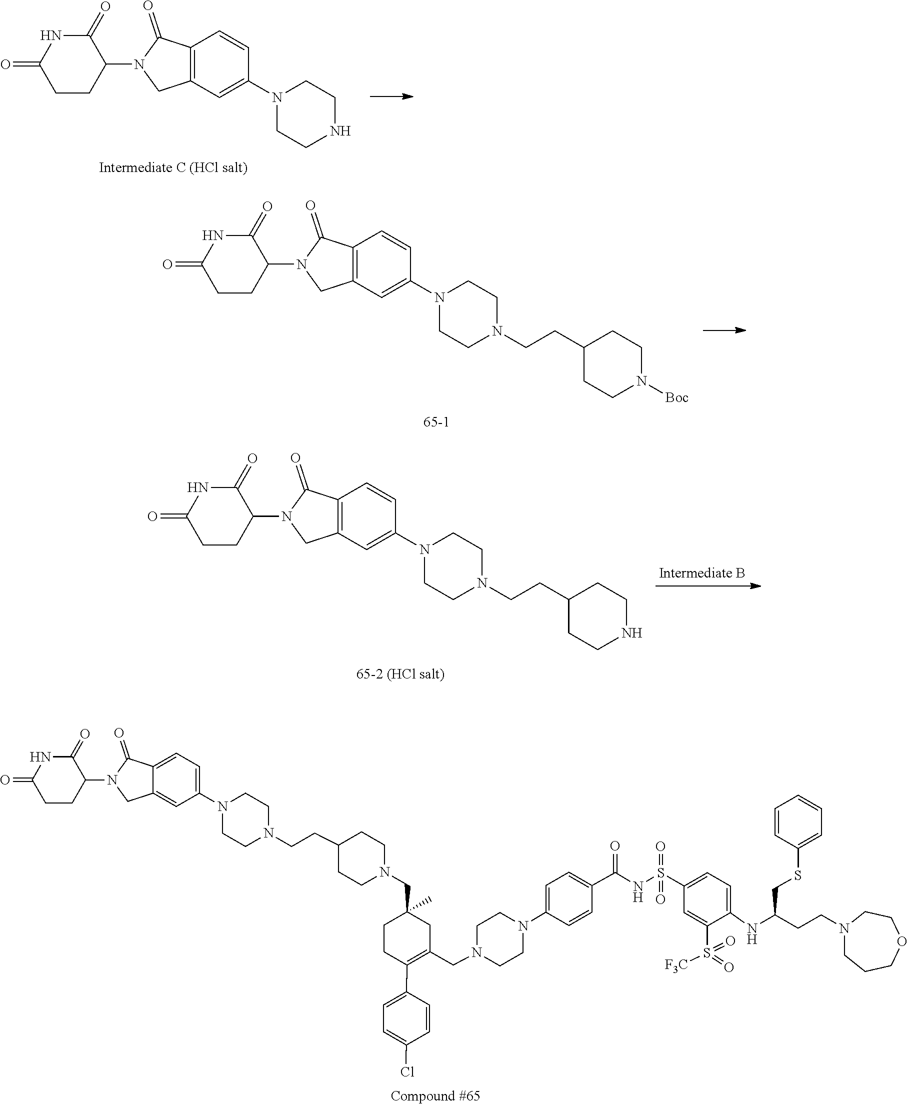 [SMILES: O=C1CCC(N2Cc3cc(N4CCNCC4)ccc3C2=O)C(=O)N1 ; CC(C)(C)OC(=O)N1CCC(CCN2CCN(c3ccc4c(c3)CN(C3CCC(=O)NC3=O)C4=O)CC2)CC1 ; O=C1CCC(N2Cc3cc(N4CCN(CCC5CCNCC5)CC4)ccc3C2=O)C(=O)N1 ; C[C@@]1(CN2CCC(CCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #65 was Prepared by Following General Procedure C:

tert-butyl 4-(2-(4-(2-(2,6-dioxopiperidin-3-yl)-1-oxoisoindolin-5-yl) piperazin-1-yl)ethyl) piperidine-1-carboxylate (65-1) (148 mg, 52% yield). ¹H NMR (600 MHz, Chloroform-d) δ 8.02 (s, 1H), 7.75 (d, J=8.5 Hz, 1H), 7.02 (dd, J=8.7, 2.2 Hz, 1H), 6.90 (d, J=2.2 Hz, 1H), 5.22 (dd, J=13.4, 5.1 Hz, 1H), 4.43 (d, J=15.6 Hz, 1H), 4.28 (d, J=15.6 Hz, 1H), 4.10 (s, 2H), 3.35 (t, J=5.2 Hz, 4H), 2.93 (ddd, J=17.8, 4.8, 2.6 Hz, 1H), 2.89-2.80 (m, 1H), 2.71 (s, 2H), 2.62 (t, J=5.0 Hz, 4H), 2.50-2.41 (m, 2H), 2.39-2.30 (m, 1H), 2.25-2.15 (m, 1H), 1.69 (d, J=13.0 Hz, 2H), 1.55-1.43 (m, 12H), 1.22-1.09 (m, 2H). LC/MS (ESI) m/z 540.3; [M+H]⁺ calcd for C₂₉H₄₂N₅O₅⁺: 540.32.

3-(1-oxo-5-(4-(2-(piperidin-4-yl)ethyl) piperazin-1-yl) isoindolin-2-yl) piperidine-2,6-dione hydrochloride (65-2) (47 mg, quantitative yield). LC/MS (ESI) m/z 440.2; [M+H]⁺ calcd for C₂₄H₃₄N₅O₃⁺: 440.27.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(4-(2-(2,6-dioxopiperidin-3-yl)-1-oxoisoindolin-5-yl) piperazin-1-yl)ethyl) piperidin-1-yl)methyl)-4-methyl-3,4,5,6-tetrahydro-[1,1′-biphenyl]-2-yl)methyl) piperazin-1-yl)benzamide 14 (compound #65) (8.52 mg, 30% yield). ¹H NMR (600 MHZ, Chloroform-d) δ 8.50 (s, 1H), 8.35 (d, J=2.2 Hz, 1H), 8.04-7.97 (m, 1H), 7.88 (d, J=8.7 Hz, 2H), 7.72 (d, J=8.6 Hz, 1H), 7.40-7.35 (m, 2H), 7.32-7.26 (m, 5H), 7.26-7.19 (m, 1H), 7.02-6.96 (m, 3H), 6.95-6.86 (m, 1H), 6.75-6.70 (m, 2H), 6.45 (d, J=9.1 Hz, 1H), 5.19 (dd, J=13.3, 5.1 Hz, 1H), 4.40 (d, J=15.7 Hz, 1H), 4.27 (d, J=15.8 Hz, 1H), 3.85-3.69 (m, 5H), 3.42-3.32 (m, 5H), 3.30-3.06 (m, 8H), 3.02 (dd, J=13.9, 6.9 Hz, 1H), 2.96-2.74 (m, 6H), 2.74-2.56 (m, 7H), 2.56-2.23 (m, 12H), 2.23-2.17 (m, 1H), 2.12 (d, J=10.0 Hz, 2H), 2.00-1.85 (m, 3H), 1.79-1.59 (m, 5H), 1.56-1.43 (m, 4H), 1.43-1.33 (m, 1H), 1.04 (s, 3H). LC/MS (ESI) m/z 1425.7; [M+H]⁺ calcd for C₇₂H₈₉ClF₃N₁₀O₉S₃⁺: 1425.56.

### Example 28: Preparation of Compound #127

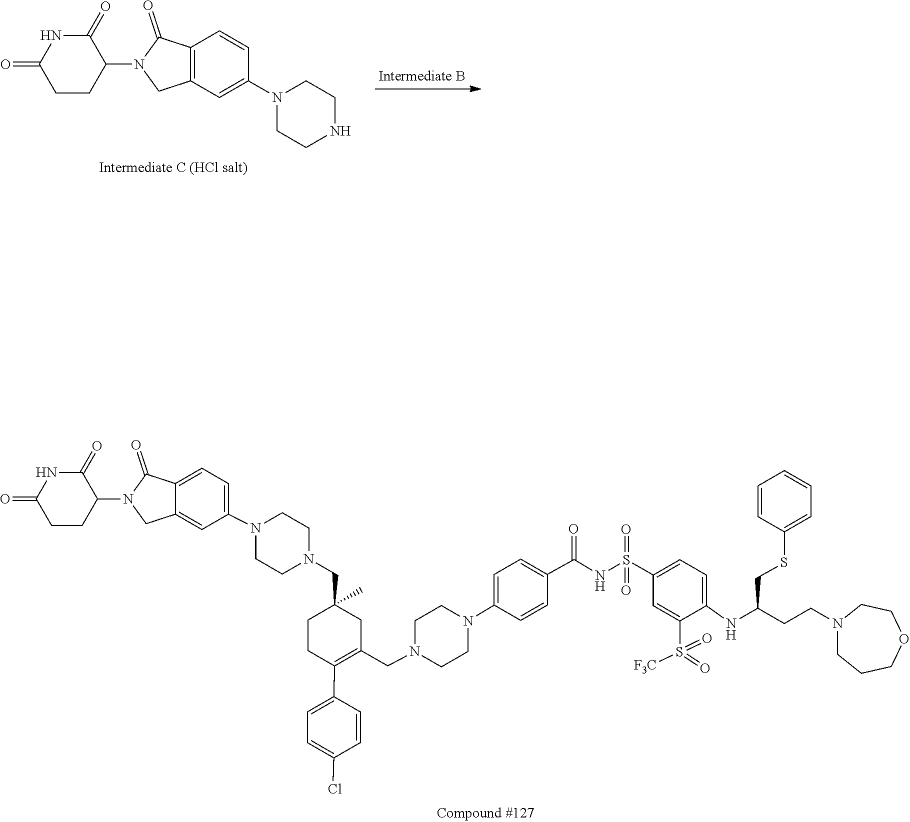 [SMILES: O=C1CCC(N2Cc3cc(N4CCNCC4)ccc3C2=O)C(=O)N1 ; C[C@@]1(CN2CCN(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(2,6-dioxopiperidin-3-yl)-1-oxoisoindolin-5-yl) piperazin-1-yl)methyl)-4-methyl-3,4,5,6-tetrahydro-[1,1′-biphenyl]-2-yl)methyl) piperazin-1-yl)benzamide (compound #127) (4.52 mg, 23% yield). The preparation of compound #127 was performed according to General Procedure H. ¹H NMR (600 MHZ, Chloroform-d) δ 8.51 (s, 1H), 8.32 (t, J=1.8 Hz, 1H), 8.08-8.00 (m, 1H), 7.78-7.70 (m, 2H), 7.66 (dd, J=18.1, 8.6 Hz, 1H), 7.38-7.33 (m, 2H), 7.28 (dt, J=7.8, 3.2 Hz, 4H), 7.25-7.20 (m, 1H), 7.05 (d, J=8.4 Hz, 1H), 7.01-6.97 (m, 2H), 6.93 (ddd, J=10.6, 8.7, 2.1 Hz, 1H), 6.83 (t, J=2.7 Hz, 1H), 6.75-6.66 (m, 2H), 6.57 (d, J=9.1 Hz, 1H), 5.20 (dd, J=12.5, 5.9 Hz, 1H), 4.38 (dd, J=15.8, 10.3 Hz, 1H), 4.23 (dd, J=16.0, 3.0 Hz, 1H), 3.88 (s, 1H), 3.81-3.67 (m, 4H), 3.33-3.16 (m, 8H), 3.08 (dd, J=13.9, 5.0 Hz, 1H), 3.02 (dd, J=13.9, 6.8 Hz, 1H), 2.96-2.57 (m, 12H), 2.53-2.37 (m, 4H), 2.37-2.21 (m, 8H), 2.21-2.13 (m, 1H), 2.13-2.04 (m, 1H), 1.95-1.86 (m, 3H), 1.79-1.70 (m, 1H), 1.68-1.58 (m, 1H), 1.49-1.40 (m, 1H), 1.01-0.94 (m, 3H). LC/MS (ESI) m/z 1314.6; [M+H]⁺ calcd for C₆₅H₇₆ClF₃N₉₀₉S₃⁺: 1314.46.

### Example 29: Preparation of Compound #128

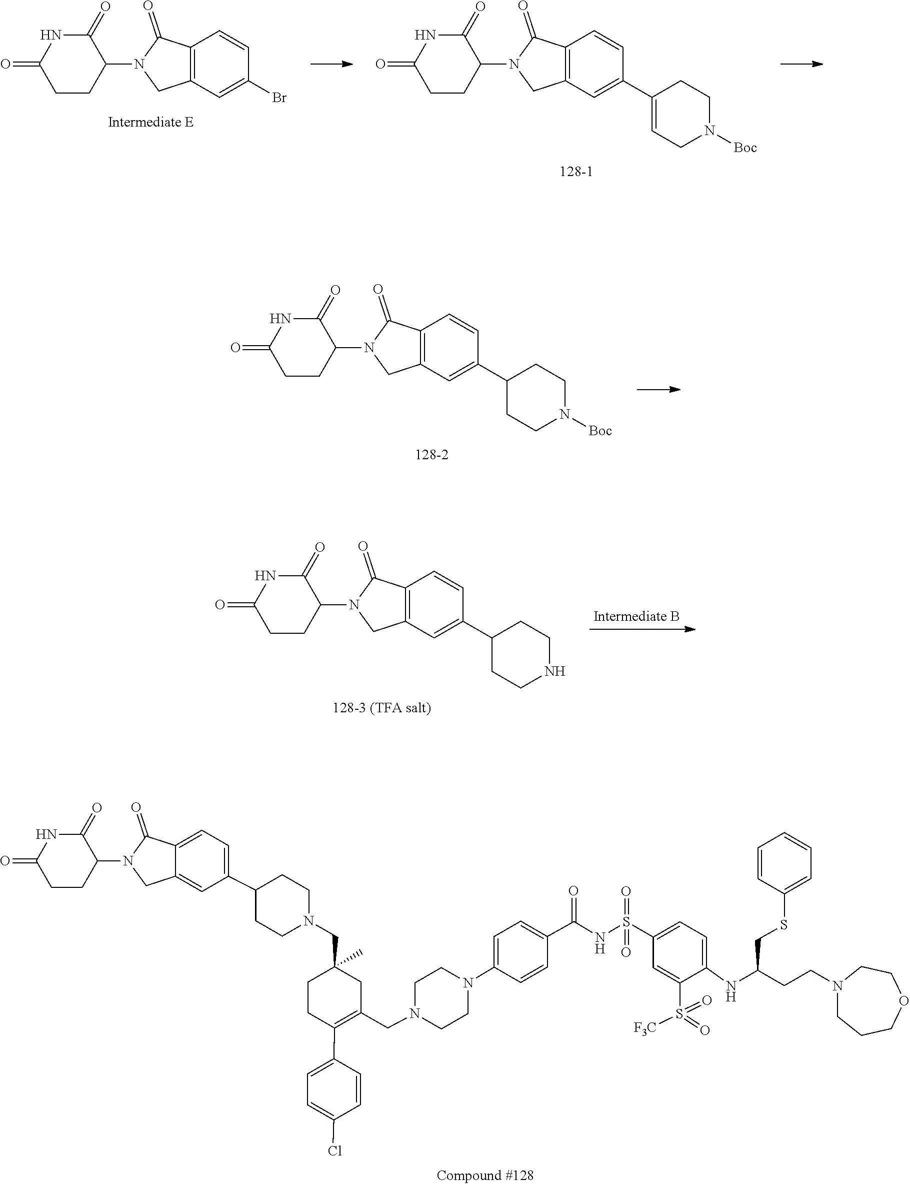 [SMILES: O=C1CCC(N2Cc3cc(Br)ccc3C2=O)C(=O)N1 ; CC(C)(C)OC(=O)N1CC=C(c2ccc3c(c2)CN(C2CCC(=O)NC2=O)C3=O)CC1 ; CC(C)(C)OC(=O)N1CCC(c2ccc3c(c2)CN(C2CCC(=O)NC2=O)C3=O)CC1 ; O=C1CCC(N2Cc3cc(C4CCNCC4)ccc3C2=O)C(=O)N1 ; C[C@@]1(CN2CCC(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Step 1: Synthesis of tert-butyl 4-(2-(2,6-dioxopiperidin-3-yl)-1-oxoisoindolin-5-yl)-3,6-dihydropyridine-1 (2H)-carboxylate (128-1). Under an argon atmosphere, a mixture of Intermediate E (600 mg, 1.86 mmol), tert-butyl 4-(4,4,5,5-tetramethyl-1,3,2-dioxaborolan-2-yl)-3,6-dihydropyridine-1 (2H)-carboxylate (631 mg, 2.04 mmol), Pd (tert-Bu3P)₂ (95 mg, 0.186 mmol) and TEA (1.3 MI, 9.29 mmol) in DMSO (20 mL) was stirred at 80° C. overnight. After cooling to room temperature, the reaction mixture was diluted with EtOAc and washed with 22 water and sat. aq. NH₄Cl. The organic layers were dried over Na₂SO₄, filtered and concentrated. The residue was purified by flash column chromatography (30% to 90% of EtOAc in hexanes) to afford 128-1 (203 mg, 26% yield). ¹H NMR (600 MHz, Chloroform-d) δ 8.04 (s, 1H), 7.86 (d, J =8.1 Hz, 1H), 7.54-7.48 (m, 2H), 7.46 (s, 1H), 5.26 (dd, J=13.3, 2.7 Hz, 1H), 4.51 (d, J=9.8 Hz, 1H), 4.38 (d, J=9.3 Hz, 1H), 4.18-4.10 (m, 2H), 2.97-2.92 (m, 2H), 2.90-2.82 (m, 2H), 2.44-2.33 (m, 2H), 2.29-2.21 (m, 2H), 1.52 (s, 9H). LC/MS (ESI) m/z 426.2; [M+H]⁺ calcd for C₂₃H₂₈N₃O₅⁺: 426.20.

Step 2: Synthesis of tert-butyl 4-(2-(2,6-dioxopiperidin-3-yl)-1-oxoisoindolin-5-yl) piperidine-1-carboxylate (128-2). A stirring mixture of 128-1 (203 mg, 0.48 mmol), and 10% palladium on carbon (40 mg) in EtOAc/MeOH (5 mL/5 mL) was hydrogenated overnight with a hydrogen balloon. The resulting mixture was filtered through celite and the filtration was concentrated. The residue was purified by flash column chromatography (30% to 90% of EtOAc in hexanes) to afford 128-2 (141 mg, 69% yield). ¹H NMR (600 MHZ, Chloroform-d) δ 8.06 (s, 1H), 7.85 (d, J=7.8 Hz, 1H), 7.36 (dd, J=7.9, 1.5 Hz, 1H), 7.32 (s, 1H), 5.25 (dd, J=13.4, 5.1 Hz, 1H), 4.49 (d, J=15.8 Hz, 1H), 4.36-4.20 (m, 3H), 3.02-2.70 (m, 4H), 2.38 (qd, J=13.3, 4.8 Hz, 1H), 2.26-2.20 (m, 1H), 1.86 (d, J=13.0 Hz, 2H), 1.73-1.58 (m, 3H), 1.51 (s, 9H). LC/MS (ESI) m/z 428.3; [M+H]⁺ calcd for C₂₃H₃₀N₃O₅⁺: 428.22.

Step 3: Synthesis of 3-(1-oxo-5-(piperidin-4-yl) isoindolin-2-yl) piperidine-2,6-dione TFA salt (128-3). 4 N HCl in dioxane (0.5 mL) was added to a stirring solution of 128-2 (15 mg, 0.037 mmol) in DCM (0.5 mL), then stirred at room temperature for 2 hours. The reaction mixture was concentrated to afford 128-3 (23 mg, quantitative yield) as TFA salt, which was directly used in the next step. LC/MS (ESI) m/z 328.1; [M+H]⁺ calcd for C₁₈H₂₂N₃O₃⁺: 328.17.

Step 4: Synthesis of N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(2,6-dioxopiperidin-3-yl)-1-oxoisoindolin-5-yl) piperidin-1-yl)methyl)-4-methyl-3,4,5,6-tetrahydro-[1,1′-biphenyl]-2-yl)methyl) piperazin-1-yl)benzamide (compound #128).

Intermediate B (20 mg, 0.02 mmol) and NaBH(OAc)₃ (5.5 mg, 0.026 mmol) were added to a solution of 128-3 (23 mg) and TEA (16.6 μL, 0.12 mmol) in DCM (1 mL), then stirred at room temperature overnight. The resulting mixture was washed with sat. aq. NH₄Cl and dried over Na₂SO₄, filtered and concentrated. The residue was purified by preparative TLC (DCM/MeOH) to afford compound #128 (1.73 mg, 7% yield). ¹H NMR (599 MHz, Chloroform-d) δ 8.36 (d, J=2.3 Hz, 1H), 8.09 (d, J=9.2 Hz, 1H), 7.81-7.68 (m, 3H), 7.43-7.23 (m, 8H), 7.17-7.09 (m, 1H), 7.03 (d, J=8.1 Hz, 2H), 6.74 (t, J=6.8 Hz, 2H), 6.58 (d, J=9.4 Hz, 1H), 5.36-5.21 (m, 1H), 4.45 (t, J=15.7 Hz, 1H), 4.31 (d, J=15.8 Hz, 1H), 3.90 (s, 1H), 3.83-3.62 (m, 4H), 3.30-3.18 (m, 4H), 3.11 (dd, J=13.9, 5.0 Hz, 1H), 3.07-2.97 (m, 3H), 2.97-2.82 (m, 4H), 2.81-2.54 (m, 7H), 2.53-2.15 (m, 13H), 2.15-2.00 (m, 1H), 1.99-1.49 (m, 9H), 1.46-1.38 (m, 1H), 0.98 (d, J=4.2 Hz, 3H). LC/MS (ESI) m/z 1313.7; [M+H]⁺ calcd for C₆₆H₇₇ClF₃N₈O₉S₃⁺: 1313.46.

### Example 30: Preparation of Compound #129

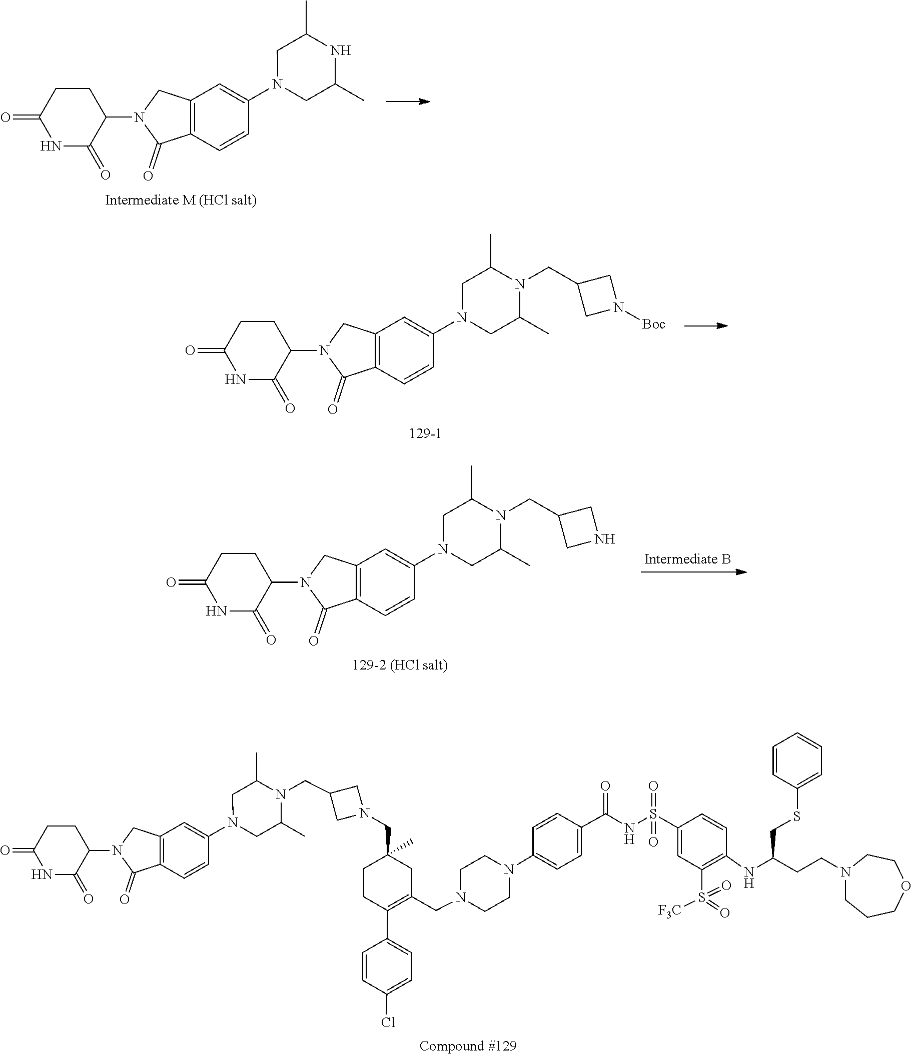 [SMILES: CC1CN(c2ccc3c(c2)CN(C2CCC(=O)NC2=O)C3=O)CC(C)N1 ; CC1CN(c2ccc3c(c2)CN(C2CCC(=O)NC2=O)C3=O)CC(C)N1CC1CN(C(=O)OC(C)(C)C)C1 ; CC1CN(c2ccc3c(c2)CN(C2CCC(=O)NC2=O)C3=O)CC(C)N1CC1CNC1 ; CC1CN(c2ccc3c(c2)CN(C2CCC(=O)NC2=O)C3=O)CC(C)N1CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)C1]

Compound #129 was prepared by following General Procedure C:

tert-butyl 3-((4-(2-(2,6-dioxopiperidin-3-yl)-1-oxoisoindolin-5-yl)-2,6-dimethylpiperazin-1-yl)methyl) azetidine-1-carboxylate (129-1) (18.5 mg, 80% yield). ¹H NMR (600 MHZ, Chloroform-d) δ 8.36 (s, 1H), 7.71 (d, J=8.5 Hz, 1H), 6.93 (dd, J=8.6, 2.2 Hz, 1H), 6.82 (d, J=2.1 Hz, 1H), 5.18 (dd, J=13.3, 5.1 Hz, 1H), 4.39 (d, J=15.6 Hz, 1H), 4.23 (d, J=15.7 Hz, 1H), 3.99 (t, J=8.4 Hz, 2H), 3.64-3.58 (m, 2H), 3.53 (d, J=10.7 Hz, 2H), 2.92 (d, J=7.1 Hz, 2H), 2.87 (ddd, J=17.8, 4.9, 2.6 Hz, 1H), 2.84-2.76 (m, 2H), 2.75-2.65 (m, 4H), 2.36-2.25 (m, 1H), 2.20-2.14 (m, 1H), 1.43 (s, 9H), 1.16 (d, J=5.8 Hz, 6H). LC/MS (ESI) m/z 526.3; [M+H]⁺ calcd for C₂₈H₄₀N₅O₅⁺: 526.30.

3-(5-(4-(azetidin-3-ylmethyl)-3,5-dimethylpiperazin-1-yl)-1-oxoisoindolin-2-yl) piperidine-2,6-dione hydrochloride (129-2) (10 mg, quantitative yield). LC/MS (ESI) m/z 426.3; [M+H]⁺ calcd for C₂₃H₃₂N₅O₃⁺: 426.25.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((3-((4-(2-(2,6-dioxopiperidin-3-yl)-1-oxoisoindolin-5-yl)-2,6-dimethylpiperazin-1-yl)methyl) azetidin-1-yl)methyl)-4-methyl-3,4,5,6-tetrahydro-[1,1′-biphenyl]-2-yl)methyl) piperazin-1-yl)benzamide (compound #129) (3.75 mg, 20% yield). ¹H NMR (600 MHZ, Chloroform-d) δ 8.35 (d, J=2.2 Hz, 1H), 7.99-7.95 (m, 1H), 7.90 (d, J=8.6 Hz, 2H), 7.67 (d, J=8.5 Hz, 1H), 7.36 (dt, J=8.0, 1.3 Hz, 2H), 7.32-7.18 (m, 5H), 6.97 (d, J=8.0 Hz, 2H), 6.90-6.82 (m, 2H), 6.78 (s, 1H), 6.65 (s, 2H), 6.40 (d, J=8.8 Hz, 1H), 5.15 (dd, J=12.8, 5.3 Hz, 1H), 4.37 (d, J=15.7 Hz, 1H), 4.23 (d, J=15.7 Hz, 1H), 3.86-3.63 (m, 5H), 3.54-3.38 (m, 2H), 3.20-3.05 (m, 2H), 3.05-2.91 (m, 8H), 2.91-2.70 (m, 8H), 2.70-2.50 (m, 10H), 2.39-2.13 (m, 11H), 2.13-2.06 (m, 1H), 1.97-1.81 (m, 3H), 1.75-1.66 (m, 1H), 1.66-1.57 (m, 1H), 1.53-1.43 (m, 1H), 1.11 (d, J=5.7 Hz, 6H), 1.07 (s, 3H). LC/MS (ESI) m/z 1411.6; [M+H]⁺ calcd for C₇₁H₈₇ClF₃N₁₀O₉S₃⁺: 1411.55.

### Example 31: Preparation of Compound #130

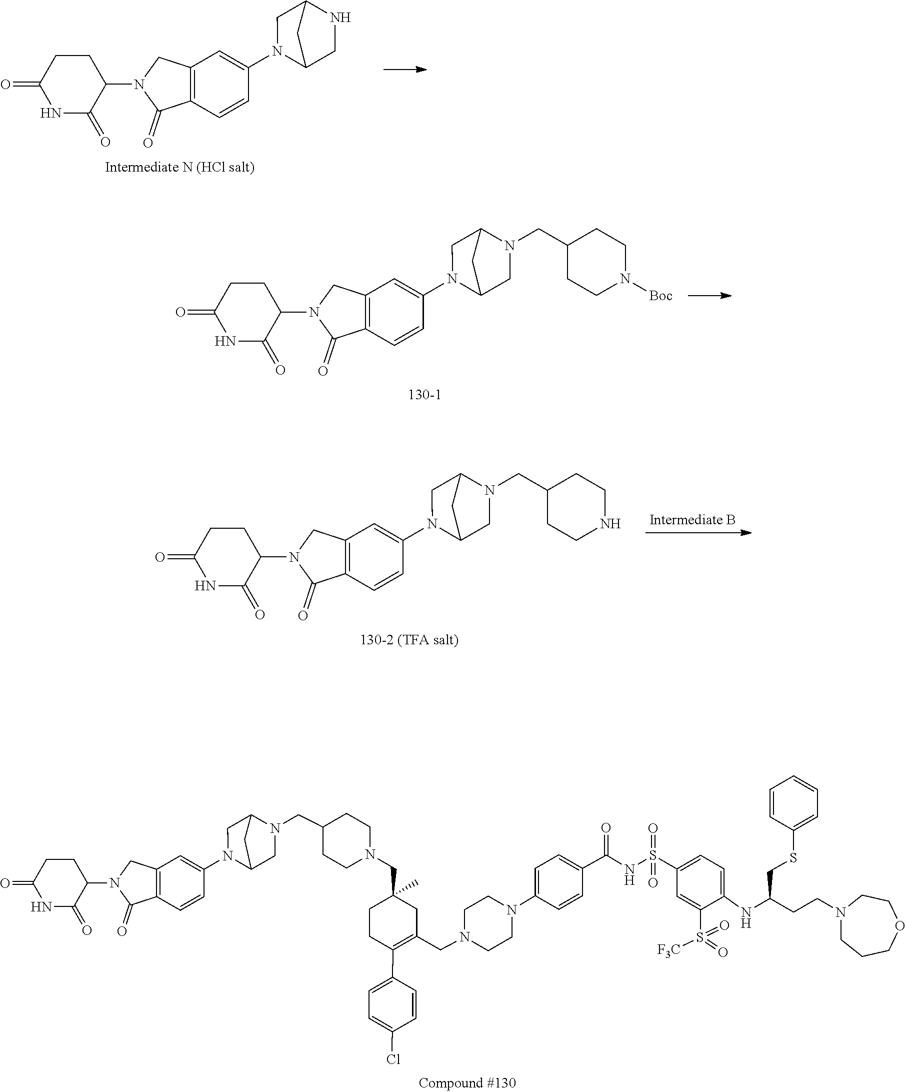 [SMILES: O=C1CCC(N2Cc3cc(N4CC5CC4CN5)ccc3C2=O)C(=O)N1 ; CC(C)(C)OC(=O)N1CCC(CN2CC3CC2CN3c2ccc3c(c2)CN(C2CCC(=O)NC2=O)C3=O)CC1 ; O=C1CCC(N2Cc3cc(N4CC5CC4CN5CC4CCNCC4)ccc3C2=O)C(=O)N1 ; C[C@@]1(CN2CCC(CN3CC4CC3CN4c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #130 was Prepared by Following General Procedure C:

tert-butyl 4-((5-(2-(2,6-dioxopiperidin-3-yl)-1-oxoisoindolin-5-yl)-2,5-diazabicyclo[2.2.1]heptan-2-yl)methyl) piperidine-1-carboxylate (130-1) (31 mg, 85% yield). 1H NMR (600 MHZ, Chloroform-d) δ 8.29 (s, 1H), 7.68 (dd, J=8.4, 1.0 Hz, 1H), 6.60 (dd, J=8.5, 2.1 Hz, 1H), 6.49 (d, J=1.9 Hz, 1H), 5.19 (ddd, J=13.3, 5.1, 2.4 Hz, 1H), 4.37 (dd, J=15.5, 4.9 Hz, 1H), 4.31-4.26 (m, 1H), 4.23 (dd, J=15.6, 3.4 Hz, 1H), 4.03 (s, 2H), 3.63 (t, J=1.9 Hz, 1H), 3.42 (dt, J=9.3, 2.4 Hz, 1H), 3.37 (d, J=9.2 Hz, 1H), 3.11-3.03 (m, 1H), 2.88 (ddd, J=17.8, 4.9, 2.6 Hz, 1H), 2.85-2.77 (m, 1H), 2.64 (s, 2H), 2.59-2.53 (m, 1H), 2.39 (dd, J=7.0, 2.9 Hz, 2H), 2.30 (qd, J=13.1, 5.0 Hz, 1H), 2.23-2.14 (m, 1H), 2.01 (d, J=9.5 Hz, 1H), 1.91 (d, J=9.6 Hz, 1H), 1.77-1.57 (m, 3H), 1.43 (s, 9H), 1.09-0.98 (m, 2H). LC/MS (ESI) m/z 538.4; [M+H]⁺ calcd for C₂₉H₄₀N₅O₅⁺: 538.30.

3-(1-oxo-5-(5-(piperidin-4-ylmethyl)-2,5-diazabicyclo[2.2.1]heptan-2-yl) isoindolin-2-yl) piperidine-2,6-dione TFA salt (130-2) (16 mg, quantitative yield). LC/MS (ESI) m/z 438.2; [M+H]⁺ calcd for C₂₄H₃₂N₅O₃⁺: 438.25.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((5-(2-(2,6-dioxopiperidin-3-yl)-1-oxoisoindolin-5-yl)-2,5-diazabicyclo[2.2.1]heptan-2-yl)methyl) piperidin-1-yl)methyl)-4-methyl-3,4,5,6-tetrahydro-[1,1′-biphenyl]-2-yl)methyl) piperazin-1-yl)benzamide (compound #130) (6.86 mg, 30% yield). ¹H NMR (600 MHz, Chloroform-d) δ 8.34 (d, J=8.2 Hz, 1H), 8.06 (d, J=9.0 Hz, 1H), 7.74 (d, J=8.3 Hz, 2H), 7.63 (d, J=8.3 Hz, 1H), 7.40-7.34 (m, 2H), 7.32-7.21 (m, 5H), 7.07-6.95 (m, 3H), 6.71 (s, 2H), 6.55 (s, 2H), 6.49 (s, 1H), 5.19 (s, 1H), 4.36 (d, J=15.5 Hz, 1H), 4.28 (s, 1H), 4.23 (d, J=15.3 Hz, 1H), 3.87 (s, 1H), 3.80-3.62 (m, 6H), 3.45-3.32 (m, 2H), 3.20 (s, 4H), 3.10 (d, J=13.9 Hz, 1H), 3.04 (d, J=6.8 Hz, 1H), 2.92-2.81 (m, 2H), 2.79-2.54 (m, 6H), 2.53-2.20 (m, 16H), 2.20-1.51 (m, 17H), 1.46-1.39 (m, 1H), 0.99 (s, 3H). LC/MS (ESI) m/z 1423.6; [M+H]⁺ calcd for C₇₂H₈₇ClF₃N₁₀O₉S₃⁺: 1423.55.

### Example 32: Preparation of Compound #131

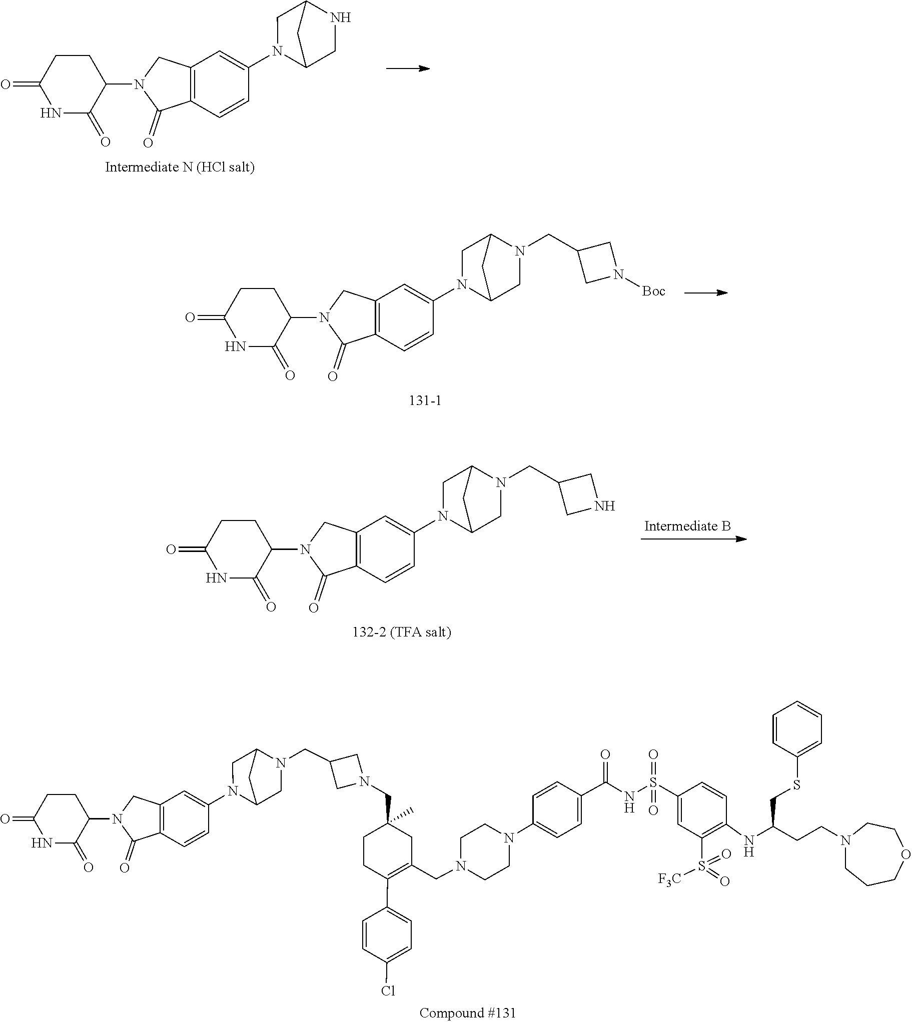 [SMILES: O=C1CCC(N2Cc3cc(N4CC5CC4CN5)ccc3C2=O)C(=O)N1 ; CC(C)(C)OC(=O)N1CC(CN2CC3CC2CN3c2ccc3c(c2)CN(C2CCC(=O)NC2=O)C3=O)C1 ; O=C1CCC(N2Cc3cc(N4CC5CC4CN5CC4CNC4)ccc3C2=O)C(=O)N1 ; C[C@@]1(CN2CC(CN3CC4CC3CN4c3ccc4c(c3)CN(C3CCC(=O)NC3=O)C4=O)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #131 was Prepared by Following General Procedure C:

tert-butyl 3-((5-(2-(2,6-dioxopiperidin-3-yl)-1-oxoisoindolin-5-yl)-2,5-diazabicyclo[2.2.1]heptan-2-yl)methyl) azetidine-1-carboxylate (131-1) (25 mg, 72% yield). ¹H NMR (600 MHZ, Chloroform-d) δ 8.42 (d, J=10.9 Hz, 1H), 7.67 (d, J=8.5 Hz, 1H), 6.59 (dt, J=8.5, 1.8 Hz, 1H), 6.49 (t, J=2.5 Hz, 1H), 5.17 (dd, J=13.4, 4.9 Hz, 1H), 4.36 (dd, J=15.5, 11.9 Hz, 1H), 4.27 (s, 1H), 4.22 (dd, J=15.7, 2.6 Hz, 1H), 3.93 (dt, J=16.8, 8.4 Hz, 2H), 3.59-3.50 (m, 3H), 3.41 (dt, J=9.1, 1.8 Hz, 1H), 3.37-3.29 (m, 1H), 2.99 (dd, J=9.3, 2.0 Hz, 1H), 2.90-2.84 (m, 1H), 2.84-2.75 (m, 1H), 2.68 (dd, J=7.6, 2.2 Hz, 2H), 2.59-2.45 (m, 2H), 2.35-2.23 (m, 1H), 2.21-2.11 (m, 1H), 1.99-1.94 (m, 1H), 1.92-1.87 (m, 1H), 1.41 (s, 9H). LC/MS (ESI) m/z 510.4; [M+H]⁺ calcd for C₂₇H₃₆N₅O₅⁺: 510.27.

3-(5-(5-(azetidin-3-ylmethyl)-2,5-diazabicyclo[2.2.1]heptan-2-yl)-1-oxoisoindolin-2-2 yl) piperidine-2,6-dione TFA salt (131-2) (17 mg, quantitative yield). LC/MS (ESI) m/z 410.2; [M+H]⁺ calcd for C₂₂H₂₈N₅O₃⁺: 410.22.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((3-((5-(2-(2,6-dioxopiperidin-3-yl)-1-oxoisoindolin-5-yl)-2,5-diazabicyclo[2.2.1]heptan-2-yl)methyl) azetidin-1-yl)methyl)-4-methyl-3,4,5,6-tetrahydro-[1,1′-biphenyl]-2-yl)methyl) piperazin-1-yl)benzamide (compound #131) (4.26 mg, 17% yield). ¹H NMR (600 MHz, Chloroform-d) δ 8.36-8.28 (m, 1H), 7.98 (d, J=9.0 Hz, 1H), 7.91 (d, J=8.3 Hz, 2H), 7.61 (d, J=8.4 Hz, 1H), 7.35 (d, J=7.6 Hz, 2H), 7.29-7.17 (m, 5H), 6.95 (d, J=8.0 Hz, 2H), 6.86 (d, J=8.3 Hz, 1H), 6.66 (d, J=8.4 Hz, 2H), 6.53-6.40 (m, 3H), 5.10 (d, 1H), 4.37-3.97 (m, 4H), 3.81 (s, 1H), 3.74 (t, J=5.9 Hz, 2H), 3.71-3.61 (m, 2H), 3.52 (s, 1H), 3.37-3.27 (m, 1H), 3.27-3.16 (m, 1H), 3.08 (dd, J=13.6, 4.7 Hz, 1H), 3.04-2.89 (m, 6H), 2.88-2.47 (m, 12H), 2.23 (s, 12H), 2.06 (d, J=23.8 Hz, 4H), 1.91 (d, J=8.9 Hz, 1H), 1.88-1.77 (m, 4H), 1.72-1.63 (m, 1H), 1.63-1.50 (m, 2H), 1.50-1.39 (m, 1H), 1.04 (s, 3H). LC/MS (ESI) m/z 1395.7; [M+H]⁺ calcd for C₇₀H₈₃ClF₃N₁₀O₉S₃⁺: 1395.51.

### Example 33: Preparation of Compound #132

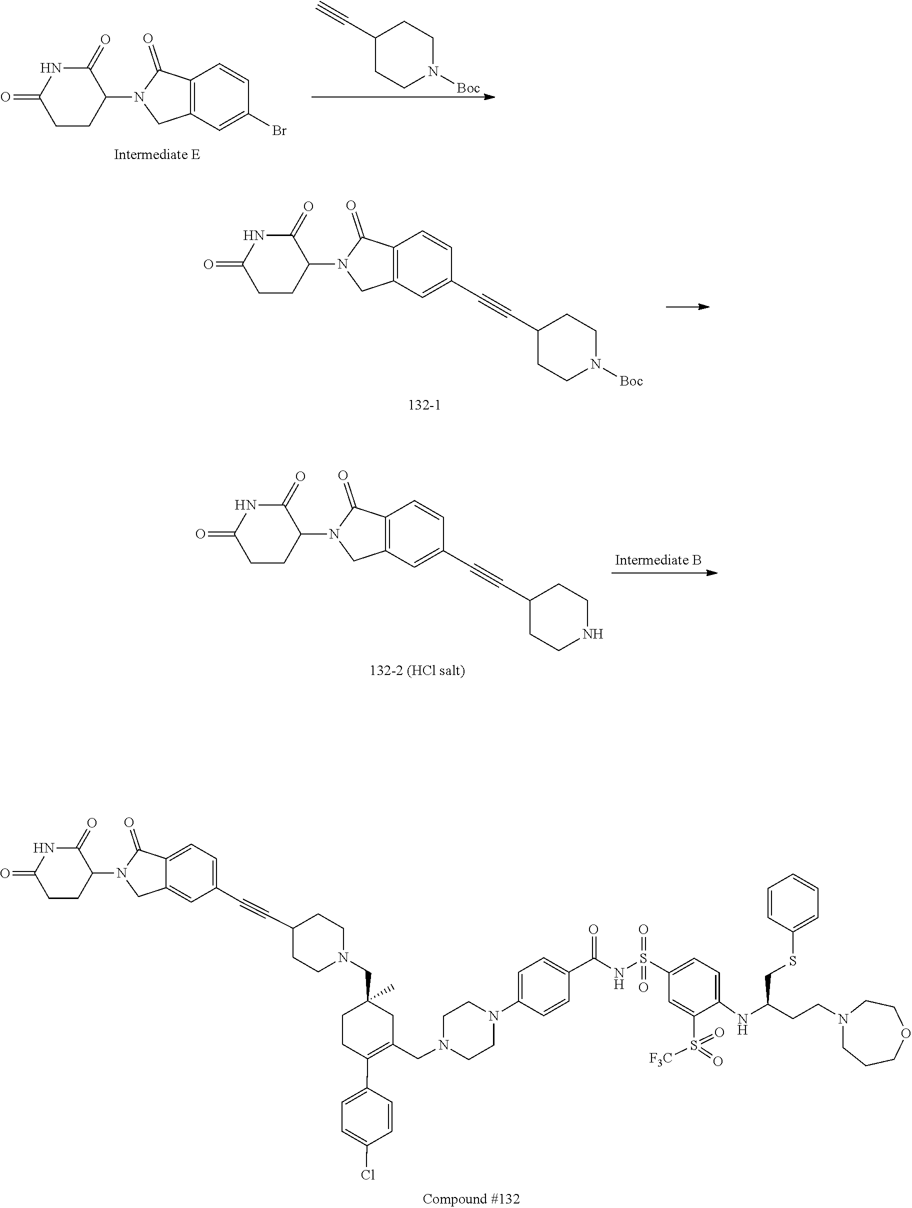 [SMILES: O=C1CCC(N2Cc3cc(Br)ccc3C2=O)C(=O)N1 ; C#CC1CCN(C(=O)OC(C)(C)C)CC1 ; CC(C)(C)OC(=O)N1CCC(C#Cc2ccc3c(c2)CN(C2CCC(=O)NC2=O)C3=O)CC1 ; O=C1CCC(N2Cc3cc(C#CC4CCNCC4)ccc3C2=O)C(=O)N1 ; C[C@@]1(CN2CCC(C#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #132 was Prepared by Following General Procedure B:

tert-butyl 4-((2-(2,6-dioxopiperidin-3-yl)-1-oxoisoindolin-5-yl) ethynyl) piperidine-1-carboxylate (132-1) (422 mg, 70% yield). ¹H NMR (600 MHZ, Chloroform-d) δ 8.00 (s, OH), 7.83 (d, J=7.8 Hz, 1H), 7.53 (dd, J=7.9, 1.3 Hz, 1H), 7.51 (s, 1H), 5.24 (dd, J=13.4, 5.1 Hz, 1H), 4.50 (d, J=15.9 Hz, 1H), 4.34 (d, J=15.9 Hz, 1H), 3.77 (d, J=10.5 Hz, 2H), 3.27 (ddd, J=13.5, 8.5, 3.4 Hz, 2H), 3.00-2.91 (m, 1H), 2.91-2.80 (m, 2H), 2.38 (qd, J=13.3, 4.7 Hz, 1H), 2.29-2.20 (m, 1H), 1.96-1.84 (m, 2H), 1.76-1.66 (m, 2H), 1.49 (s, 9H). LC/MS (ESI) m/z 452.3; [M+H]⁺ calcd for C₂₅H₃₀N₃O₅⁺: 452.22.

3-(1-oxo-5-(piperidin-4-ylethynyl) isoindolin-2-yl) piperidine-2,6-dione hydrochloride (132-2) (22 mg, quantitative yield). LC/MS (ESI) m/z 352.2; [M+H]⁺ calcd for C₂₀H₂₂N₃O₃⁺: 352.17.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((2-(2,6-dioxopiperidin-3-yl)-1-oxoisoindolin-5-yl) ethynyl) piperidin-1-yl)methyl)-4-methyl-3,4,5,6-tetrahydro-[1,1′-biphenyl]-2-yl)methyl) piperazin-1-yl)benzamide (compound #132) (5.1 mg, % yield). ¹H NMR (600 MHZ, Chloroform-d) δ 8.36 (dd, J=5.5, 2.3 Hz, 1H), 8.06 (d, J=9.2 Hz, 1H), 7.82-7.71 (m, 3H), 7.53-7.44 (m, 2H), 7.41-7.35 (m, 2H), 7.34-7.21 (m, 6H), 7.09-6.96 (m, 3H), 6.75 (d, J=8.7 Hz, 2H), 6.55 (d, J=9.3 Hz, 1H), 5.29-5.20 (m, 1H), 4.47 (dd, J=16.2, 4.4 Hz, 1H), 4.33 (d, J=16.2 Hz, 1H), 3.88 (s, 1H), 3.82-3.71 (m, 4H), 3.25 (s, 4H), 3.11 (dd, J=13.9, 5.0 Hz, 1H), 3.05 (dd, J=14.0, 6.8 Hz, 1H), 2.99-2.84 (m, 6H), 2.84-2.74 (m, 4H), 2.75-2.52 (m, 5H), 2.48-2.19 (m, 11H), 2.16-2.06 (m, 1H), 2.05-1.89 (m, 4H), 1.88-1.62 (m, 5H), 1.50-1.41 (m, 1H), 0.99 (s, 3H). LC/MS (ESI) m/z 1337.6; [M+H]⁺ calcd for C₆₈H₇₇ClF₃N₈O₉S₃⁺: 1337.46.

### Example 34: Preparation of Compound #133

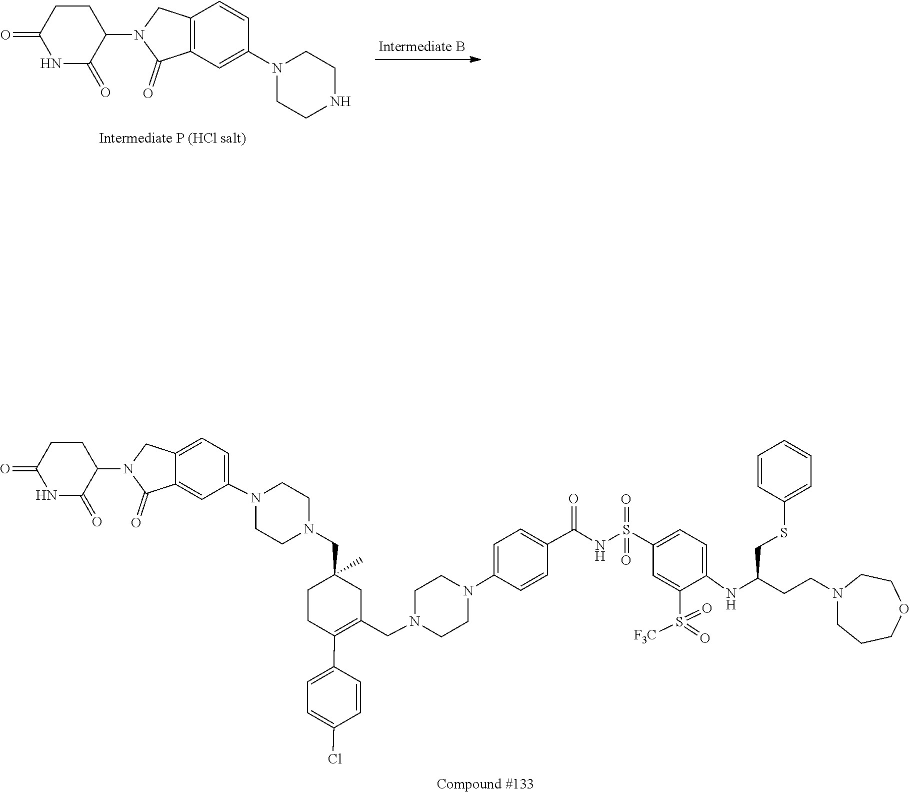 [SMILES: O=C1CCC(N2Cc3ccc(N4CCNCC4)cc3C2=O)C(=O)N1 ; C[C@@]1(CN2CCN(c3ccc4c(c3)C(=O)N(C3CCC(=O)NC3=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(2,6-dioxopiperidin-3-yl)-3-oxoisoindolin-5-yl) piperazin-1-yl)methyl)-4-methyl-3,4,5,6-tetrahydro-[1,1′-biphenyl]-2-yl)methyl) piperazin-1-yl)benzamide (compound #132) (7.34 mg, 28% yield). The preparation of compound #132 was performed according to General Procedure H. ¹H NMR (600 MHZ, Chloroform-d) δ 8.27 (t, J=1.9 Hz, 1H), 8.10-8.03 (m, 1H), 7.90-7.81 (m, 2H), 7.38-7.30 (m, 3H), 7.31-7.15 (m, 7H), 7.02-6.95 (m, 2H), 6.83-6.77 (m, 2H), 6.58 (dd, J=9.4, 5.4 Hz, 1H), 5.65-5.46 (m, 1H), 4.37 (dd, J=15.7, 3.2 Hz, 1H), 4.29 (dd, J=15.8, 4.4 Hz, 1H), 3.94-3.80 (m, 1H), 3.78-3.70 (m, 2H), 3.70-3.58 (m, 2H), 3.19 (t, J=5.1 Hz, 8H), 3.07 (ddd, J=13.8, 5.2, 2.6 Hz, 1H), 3.04-2.99 (m, 1H), 2.98-2.90 (m, 1H), 2.90-2.77 (m, 3H), 2.78-2.70 (m, 4H), 2.69-2.63 (m, 2H), 2.63-2.43 (m, 5H), 2.41-2.15 (m, 10H), 2.07-1.97 (m, 1H), 1.85-1.79 (m, 2H), 1.79-1.36 (m, 4H), 0.94 (d, J=15.5 Hz, 3H). LC/MS (ESI) m/z 1314.5; [M+H]⁺ calcd for C₆₅H₇₆ClF₃N₉O₉S₃⁺: 1314.46.

### Example 35: Preparation of Compound #135

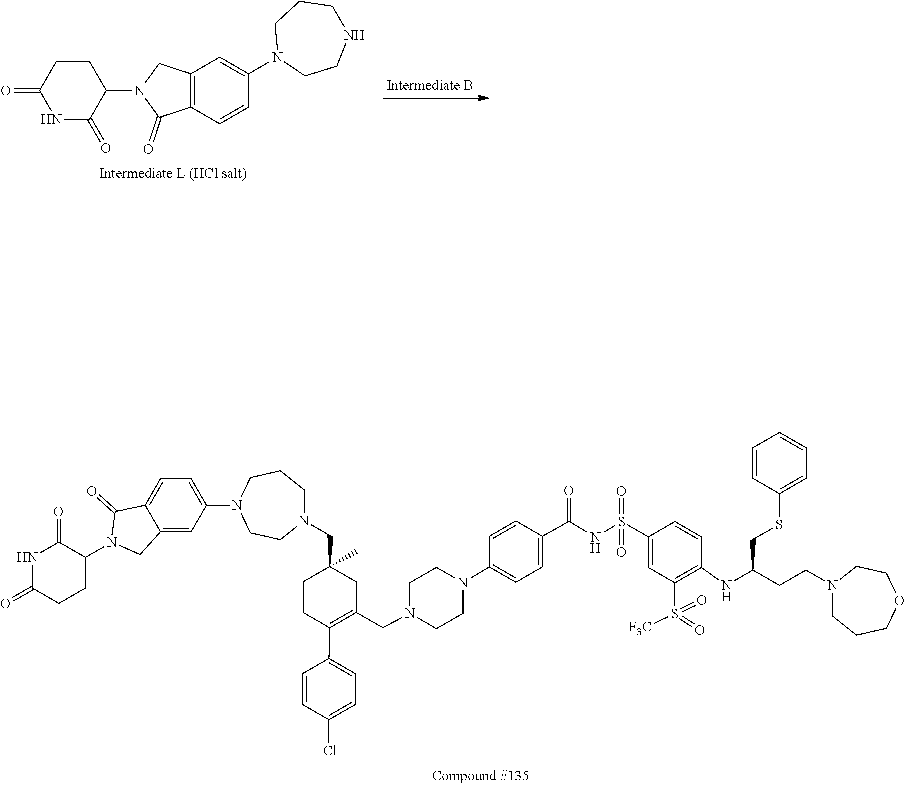 [SMILES: O=C1CCC(N2Cc3cc(N4CCCNCC4)ccc3C2=O)C(=O)N1 ; C[C@@]1(CN2CCCN(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(2-(2,6-dioxopiperidin-3-yl)-1-oxoisoindolin-5-yl)-1,4-diazepan-1-yl)methyl)-4-methyl-3,4,5,6-tetrahydro-[1,1′-biphenyl]-2-yl)methyl) piperazin-1-yl)benzamide (compound #135) (8.63 mg, 28% yield). The preparation of compound #135 was performed according to General Procedure H. ¹H NMR (600 MHZ, Chloroform-d) δ 8.31 (t, J=2.5 Hz, 1H), 8.06 (dd, J=9.3, 2.2 Hz, 1H), 7.72 (dd, J=8.8, 4.0 Hz, 2H), 7.47 (t, J=8.2 Hz, 1H), 7.37-7.31 (m, 2H), 7.30-7.23 (m, 4H), 7.23-7.19 (m, 1H), 6.96 (d, J=8.3 Hz, 2H), 6.73-6.64 (m, 3H), 6.62 (t, J=2.9 Hz, 1H), 6.59-6.52 (m, 1H), 5.23-5.11 (m, 1H), 4.32 (dd, J=15.7, 7.7 Hz, 1H), 4.17 (d, J=15.7 Hz, 1H), 3.89-3.82 (m, 1H), 3.74 (t, J=6.1 Hz, 2H), 3.72-3.64 (m, 2H), 3.59-3.47 (m, 4H), 3.15 (t, J=5.2 Hz, 4H), 3.07 (dd, J=13.9, 5.0 Hz, 1H), 3.01 (dd, J=13.9, 7.0 Hz, 1H), 2.98-2.89 (m, 2H), 2.87-2.76 (m, 4H), 2.76-2.50 (m, 8H), 2.43 (dd, J=14.1, 2.6 Hz, 1H), 2.40-2.15 (m, 8H), 2.15-2.09 (m, 1H), 2.09-2.02 (m, 1H), 1.96-1.83 (m, 4H), 1.75-1.50 (m, 4H), 1.39-1.33 (m, 1H), 0.90 (s, 3H). LC/MS (ESI) m/z 1328.6; [M+H]⁺ calcd for C₆₆H₇₈ClF₃N₉₀₉S₃⁺: 1328.47.

### Example 36: Preparation of Compound #136

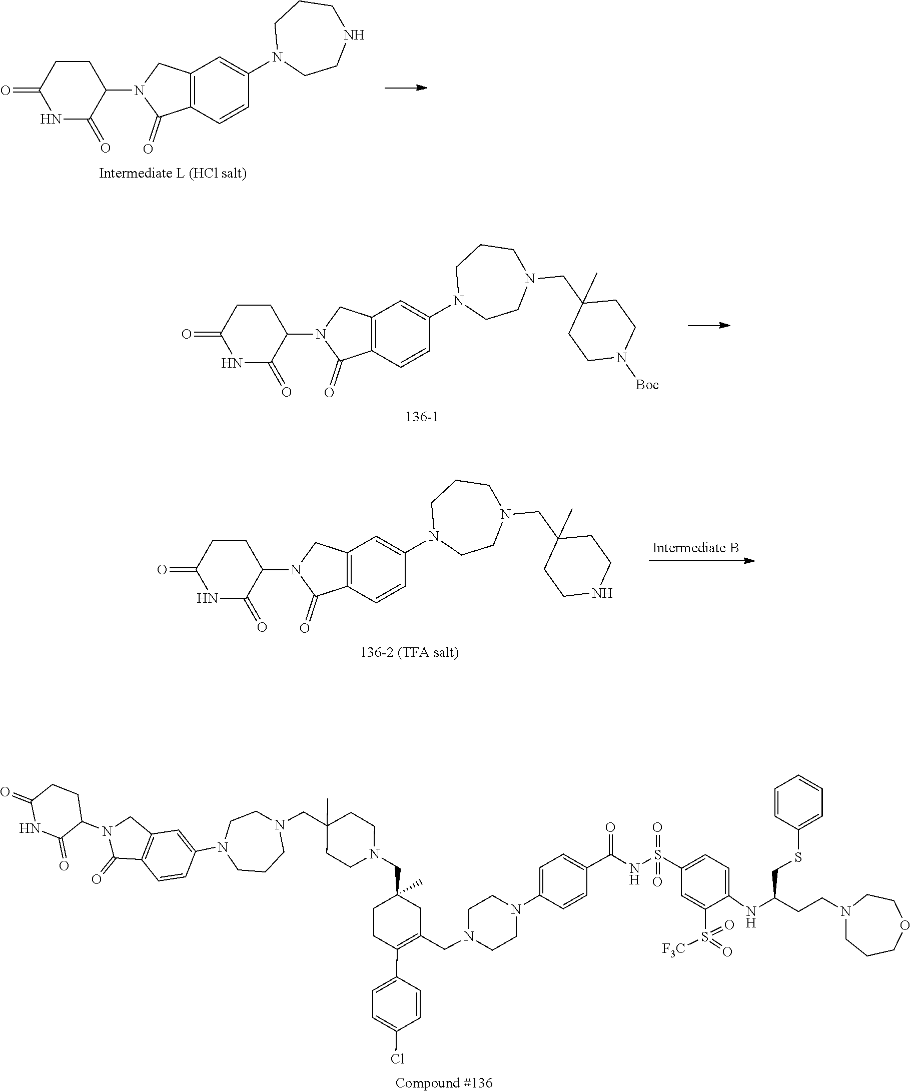 [SMILES: O=C1CCC(N2Cc3cc(N4CCCNCC4)ccc3C2=O)C(=O)N1 ; CC1(CN2CCCN(c3ccc4c(c3)CN(C3CCC(=O)NC3=O)C4=O)CC2)CCN(C(=O)OC(C)(C)C)CC1 ; CC1(CN2CCCN(c3ccc4c(c3)CN(C3CCC(=O)NC3=O)C4=O)CC2)CCNCC1 ; CC1(CN2CCCN(c3ccc4c(c3)CN(C3CCC(=O)NC3=O)C4=O)CC2)CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1]

Compound #136 was prepared by following General Procedure C:

tert-butyl 4-((4-(2-(2,6-dioxopiperidin-3-yl)-1-oxoisoindolin-5-yl)-1,4-diazepan-1-yl)methyl)-4-methylpiperidine-1-carboxylate (136-1) (24 mg, 38% yield). ¹H NMR (600 MHZ, Chloroform-d) δ 8.09 (s, 1H), 7.67 (d, J=8.7 Hz, 1H), 6.74 (dd, J=8.7, 2.2 Hz, 1H), 6.63 (d, J=2.2 Hz, 1H), 5.19 (dd, J=13.3, 5.1 Hz, 1H), 4.38 (d, J=15.4 Hz, 1H), 4.23 (d, J=15.5 Hz, 1H), 3.74-3.63 (m, 2H), 3.58 (t, J=6.3 Hz, 2H), 3.55 (t, J=5.0 Hz, 2H), 3.05 (t, J=12.1 Hz, 2H), 2.92-2.86 (m, 3H), 2.82 (ddd, J=18.0, 13.2, 5.3 Hz, 1H), 2.67 (t, J=6.8, 4.6 Hz, 2H), 2.35-2.25 (m, 3H), 2.22-2.14 (m, 1H), 1.91 (p, J=6.1 Hz, 2H), 1.45 (s, 9H), 1.43-1.35 (m, 2H), 1.27-1.17 (m, 2H), 0.91 (s, 3H). LC/MS (ESI) m/z 554.4; [M+H]⁺ calcd for C₃₀H₄₄N₅O₅⁺: 554.33.

3-(5-(4-((4-methylpiperidin-4-yl)methyl)-1,4-diazepan-1-yl)-1-oxoisoindolin-2-yl) piperidine-2,6-dione TFA salt (136-2) (14 mg, quantitative yield). LC/MS (ESI) m/z 454.2; [M+H]⁺ calcd for C₂₅H₃₆N₅O₃⁺: 454.28.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-(2-(2,6-dioxopiperidin-3-yl)-1-oxoisoindolin-5-yl)-1,4-diazepan-1-yl)methyl)-4-methylpiperidin-1-yl)methyl)-4-methyl-3,4,5,6-tetrahydro-[1,1′-biphenyl]-2-yl)methyl) piperazin-1-yl)benzamide (compound #136) (6.89 mg, 32% yield). ¹H NMR (600 MHZ, Chloroform-d) δ 8.31 (d, J=2.3 Hz, 1H), 8.00 (dd, J=9.1, 2.2 Hz, 1H), 7.82 (d, J=8.4 Hz, 2H), 7.61 (dd, J=8.7, 3.4 Hz, 1H), 7.38-7.32 (m, 2H), 7.31-7.23 (m, 5H), 7.23-7.18 (m, 1H), 6.97 (d, J=8.0 Hz, 2H), 6.67 (d, J=8.1 Hz, 3H), 6.59 (s, 1H), 6.46 (d, J=9.2 Hz, 1H), 5.12 (dt, J=13.3, 6.8 Hz, 1H), 4.33 (d, J=15.6 Hz, 1H), 4.21 (d, J=15.7 Hz, 1H), 3.79 (s, 1H), 3.75 (t, J=6.1 Hz, 2H), 3.71-3.65 (m, 2H), 3.55-3.43 (m, 4H), 3.23-3.04 (m, 6H), 3.00 (dd, J=13.8, 7.0 Hz, 1H), 2.91-2.50 (m, 17H), 2.47-2.20 (m, 10H), 2.19-2.10 (m, 1H), 2.10-2.01 (m, 1H), 1.93-1.76 (m, 4H), 1.78-1.41 (m, 9H), 1.05 (s, 3H), 0.87 (t, J=1.6 Hz, 3H). LC/MS (ESI) m/z 1439.7; [M+H]⁺ calcd for C₇₃H₉₁ClF₃N₁₀O₉S₃⁺: 1439.58.

### Example 37: Preparation of Compound #137

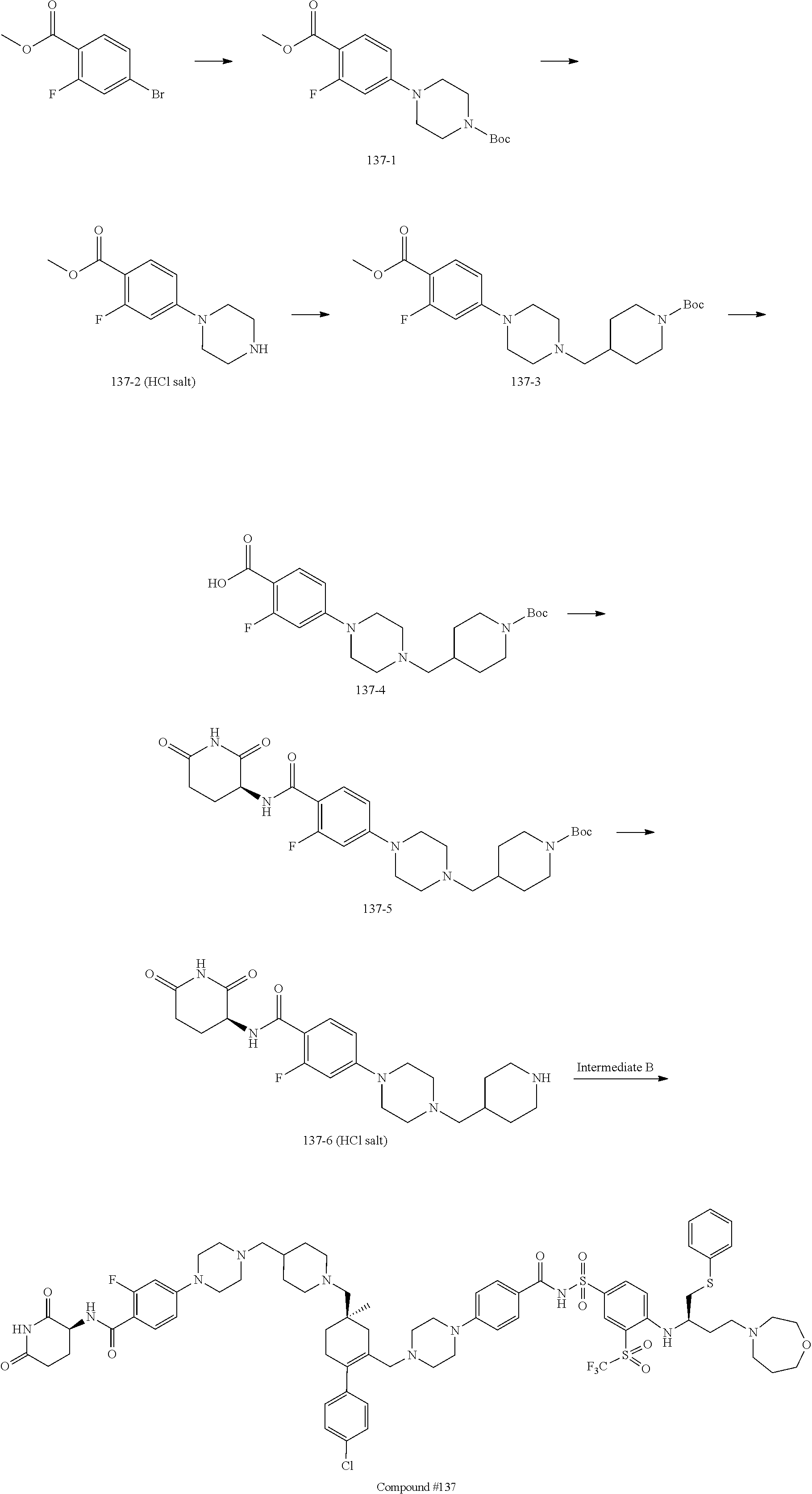

Step 1: Synthesis of tert-butyl 4-(3-fluoro-4-(methoxycarbonyl)phenyl) piperazine-1-carboxylate (137-1). Under an argon atmosphere, a mixture of methyl 4-bromo-2-fluorobenzoate (1 g, 4.29 mmol), 4-Boc-piperazine (1.12 g, 6.01 mmol), Pd(OAc)₂ (48 mg, 0.21 mmol), BINAP (267 mg, 0.43 mmol) and Cs₂CO₃ (1.54 g, 4.72 mmol) in toluene (20 mL) was stirred at 60° C. overnight. After cooling to room temperature, the reaction mixture was washed with water and sat. aq. NH₄Cl. The organic layers were dried over Na₂SO₄, filtered and concentrated. The residue was purified by flash column chromatography (10% to 40% of EtOAc in hexanes) to afford 137-1 (410 mg, 37% yield). ¹H NMR (600 MHZ, Chloroform-d) δ 7.83 (t, J=8.8 Hz, 1H), 6.61 (dd, J=9.0, 2.5 Hz, 1H), 6.50 (dd, J=14.5, 2.5 Hz, 1H), 3.87 (s, 3H), 3.57 (t, J=6.5, 4.1 Hz, 4H), 3.30 (t, J=5.3 Hz, 4H), 1.48 (s, 9H). LC/MS (ESI) m/z 339.2; [M+H]⁺ calcd for C₁₇H₂₄FN₂O₄⁺: 339.17.

Step 2: Synthesis of methyl 2-fluoro-4-(piperazin-1-yl)benzoate hydrochloride (137-2). 4 N HCl in dioxane (5 mL) was added to a stirring solution of 137-1 (200 mg, 0.59 mmol) in DCM (5 mL) at room temperature. The resulting mixture was stirred at room temperature for 2 hours, then concentrated to afford 137-2 (195 mg, quantitative yield), which was directly used in the next step. LC/MS (ESI) m/z 239.2; [M+H]⁺ calcd for C₁₂H₁₆FN₂O₂⁺: 239.12.

Step 3: Synthesis of tert-butyl 4-((4-(3-fluoro-4-(methoxycarbonyl)phenyl) piperazin-1-yl)methyl) piperidine-1-carboxylate (137-3). tert-Butyl 4-formylpiperidine-1-carboxylate (189 mg, 0.89 mmol) and NaBH(OAc)₃ (188 mg, 0.89 mmol) were added to a solution of 137-2 (195 mg, 0.71 mmol) and TEA (789 μL, 5.67 mmol) in DCM (10 mL), then stirred at room temperature overnight. The resulting mixture was washed with water and sat. aq. NH₄Cl. The organic layers were dried over Na₂SO₄, filtered and concentrated. The residue was purified by flash column chromatography (10% to 40% of EtOAc in hexanes) to afford 137-3 (218 mg, 85% 10 yield in two steps). ¹H NMR (600 MHZ, Chloroform-d) δ 7.84 (t, J=8.8 Hz, 1H), 6.64 (dd, J=8.9, 2.5 Hz, 1H), 6.52 (dd, J=14.7, 2.4 Hz, 1H), 4.12 (s, 2H), 3.90 (s, 3H), 3.33 (t, 4H), 2.72 (s, 2H), 2.55 (t, 4H), 2.24 (d, J=7.2 Hz, 2H), 1.77 (d, J=11.8 Hz, 2H), 1.73-1.63 (m, 1H), 1.48 (s, 9H), 1.11 (qd, J=12.4, 4.3 Hz, 2H). LC/MS (ESI) m/z 436.3; [M+H]⁺ calcd for C₂₃H₃₅FN₃O₄⁺: 436.26.

Step 4: Synthesis of 4-(4-((1-(tert-butoxycarbonyl) piperidin-4-yl)methyl) piperazin-1-yl)-2-fluorobenzoic acid (137-4). To a solution of 137-3 (210 mg, 0.48 mmol) in MeOH/THF (5 mL/5 mL) was added a solution of LiOH·H₂O (61 mg, 0.14 mmol) in water (1 mL), then stirred at room temperature overnight. The reaction mixture was concentrated to remove the organic solvents, then adjusted to pH=6 with 10% aq. citric acid and extracted with EtOAc. The combined organic layers were washed with brine and dried over Na₂SO₄, filtered and concentrated. The residue was purified by flash column chromatography (10% to 40% of EtOAc in hexanes) to afford 137-4 (179 mg, 88% yield). ¹H NMR (600 MHZ, Chloroform-d) δ 7.89 (t, J=8.9 Hz, 1H), 6.65 (dd, J=9.1, 2.4 Hz, 1H), 6.53 (dd, J=14.8, 2.4 Hz, 1H), 4.12 (s, 2H), 3.37 (t, J=5.1 Hz, 4H), 2.73 (s, 2H), 2.58 (t, J=5.1 Hz, 4H), 2.28 (d, J=7.0 Hz, 2H), 1.77 (d, J=11.7 Hz, 2H), 1.74-1.63 (m, 1H), 1.48 (s, 9H), 1.13 (qd, J=12.5, 4.3 Hz, 2H). LC/MS (ESI) m/z 422.2; [M+H]⁺ calcd for C₂₂H₃₃FN3O₄⁺: 422.24.

Step 5: Synthesis of tert-butyl(S)-4-((4-(4-((2,6-dioxopiperidin-3-yl) carbamoyl)-3-fluorophenyl) piperazin-1-yl)methyl) piperidine-1-carboxylate (137-5). To a solution of 137-4 (18 mg, 0.043 mmol) and HATU (21 mg, 0.055 mmol) in (1 mL) was added a solution of(S)-3-aminopiperidine-2,6-dione hydrochloride (8 mg, 0.048 mmol) and DIPEA (30 μL, 0.17 mmol) in DCM (0.5 mL). The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH=10/1) to afford 137-5 (14 mg, 62% yield). ¹H NMR (600 MHZ, Chloroform-d) δ 8.31 (s, 1H), 7.94 (t, J=9.2 Hz, 1H), 7.41 (dd, J=14.1, 5.7 Hz, 1H), 6.70 (dd, J=9.0, 2.4 Hz, 1H), 6.50 (dd, J=16.3, 2.4 Hz, 1H), 4.84-4.70 (m, 1H), 4.09 (s, 2H), 3.30 (t, J=5.1 Hz, 4H), 2.87-2.72 (m, 2H), 2.73-2.63 (m, 3H), 2.53 (t, J=5.1 Hz, 4H), 2.22 (d, J=7.1 Hz, 2H), 1.94 (qd, J=12.9, 5.2 Hz, 1H), 1.79-1.70 (m, 2H), 1.70-1.59 (m, 1H), 1.45 (s, 9H), 1.09 (qd, J=12.5, 4.3 Hz, 2H). LC/MS (ESI) m/z 532.4; [M+H]⁺ calcd for C₂₇H₃₉FN₅O₅⁺: 532.29.

Step 6: Synthesis of(S)—N-(2,6-dioxopiperidin-3-yl)-2-fluoro-4-(4-(piperidin-4-ylmethyl) piperazin-1-yl)benzamide hydrochloride (137-6). To a stirring solution of 137-5 (14 mg, 0.026 mmol) in DCM (0.5 mL) was added 4 N HCl in dioxane (0.5 mL) and stirred at room temperature for 2 hours. The reaction mixture was concentrated to afford 137-6 (13 mg, quantitative yield), which was directly used in the next step. LC/MS (ESI) m/z 432.3; [M+H]⁺ calcd for C₂₂H₃₁FN₅O₃⁺: 432.24.

Step 7: Synthesis of 4-(4-((1-(((R)-6-((4-(4-(((4-(((R)-4-(1,4-oxazepan-4-yl)-1-(phenylthio) butan-2-yl)amino)-3-((trifluoromethyl) sulfonyl)phenyl) sulfonyl) carbamoyl)phenyl) piperazin-1-yl)methyl)-4′-chloro-4-methyl-2,3,4,5-tetrahydro-[1,1′-biphenyl]-4-yl)methyl) piperidin-4-yl)methyl) piperazin-1-yl)-N-((S)-2,6-dioxopiperidin-3-yl)-2-fluorobenzamide (compound 137). Intermediate B (17 mg, 0.017 mmol) and NaBH(OAc)₃ (4.3 mg, 0.02 mmol) were added to a solution of 137-6 (13 mg, 0.027 mmol) and TEA (19 μL, 0.14 mmol) in DCM (1 mL), then stirred at room temperature overnight. The resulting mixture was washed with sat. aq. NH₄Cl and dried over Na₂SO₄, filtered and concentrated. The residue was purified by preparative TLC (DCM/MeOH) to afford compound #137 (4.78 mg, 20% yield in two steps).1H NMR (600 MHZ, Chloroform-d) δ 8.34 (d, J=2.2 Hz, 1H), 8.07 (s, 1H), 8.02 (d, J=9.1 Hz, 1H), 7.93 (t, J=9.2 Hz, 1H), 7.77 (d, J=8.5 Hz, 2H), 7.45-7.34 (m, 3H), 7.28 (s, 5H), 6.98 (d, J=8.2 Hz, 3H), 6.72 (d, J=8.1 Hz, 2H), 6.68 (dd, J=9.0, 2.3 Hz, 1H), 6.53-6.44 (m, 2H), 4.82-4.72 (m, 1H), 3.83 (s, 1H), 3.80-3.66 (m, 4H), 3.24 (d, J=31.7 Hz, 9H), 3.09 (dd, J=13.9, 5.0 Hz, 1H), 3.02 (dd, J=13.9, 7.0 Hz, 1H), 2.90-2.64 (m, 6H), 2.63-2.56 (m, 1H), 2.51 (s, 4H), 2.47-2.18 (m, 12H), 2.14-1.83 (m, 12H), 1.80-1.67 (m, 2H), 1.67-1.53 (m, 3H), 1.52-1.41 (m, 1H), 1.03 (s, 3H).). LC/MS (ESI) m/z 1417.6; [M+H]⁺ calcd for C₇₀H₈₆ClF₄N₁₀O₉S₃⁺: 1417.54.

### Example 38: Preparation of Compound #138

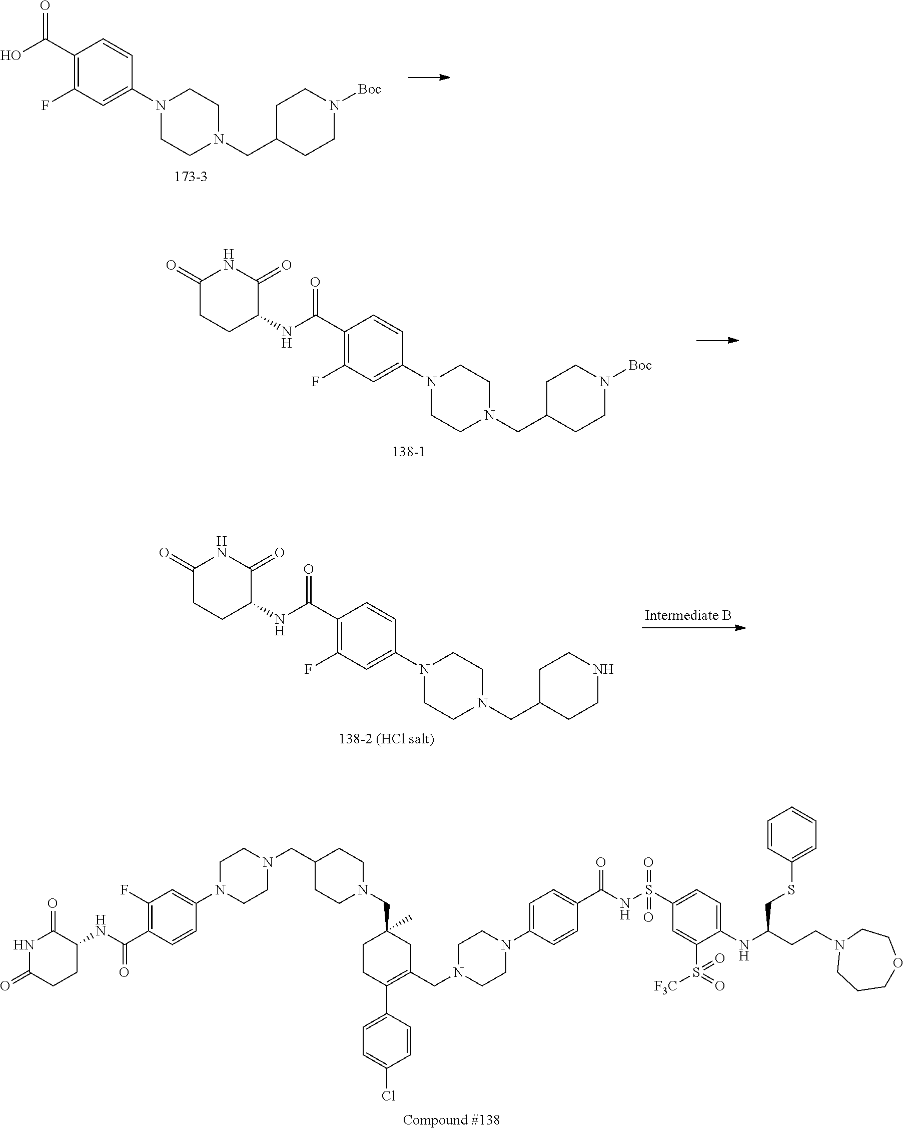 [SMILES: CC(C)(C)OC(=O)N1CCC(CN2CCN(c3ccc(C(=O)O)c(F)c3)CC2)CC1 ; CC(C)(C)OC(=O)N1CCC(CN2CCN(c3ccc(C(=O)N[C@@H]4CCC(=O)NC4=O)c(F)c3)CC2)CC1 ; O=C1CC[C@@H](NC(=O)c2ccc(N3CCN(CC4CCNCC4)CC3)cc2F)C(=O)N1 ; C[C@@]1(CN2CCC(CN3CCN(c4ccc(C(=O)N[C@@H]5CCC(=O)NC5=O)c(F)c4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Step 1: Synthesis of tert-butyl (R)-4-((4-(4-((2,6-dioxopiperidin-3-yl) carbamoyl)-3-fluorophenyl) piperazin-1-yl)methyl) piperidine-1-carboxylate (138-1). To a solution of 137-4 (15 mg, 0.036 mmol) and HATU (17.6 mg, 0.046 mmol) in (1 mL) was added a solution of (R)-3-aminopiperidine-2,6-dione hydrochloride (7 mg, 0.042 mmol) and DIPEA (25 μL, 0.14 mmol) in DCM (0.5 mL). The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH=10/1) to afford 138-1 (13 mg, 69% yield). ¹H NMR (600 MHZ, Chloroform-d) δ 8.03 (s, 1H), 7.95 (t, J=9.2 Hz, 1H), 7.40 (dd, J=14.2, 5.7 Hz, 1H), 6.70 (dd, J=9.0, 2.4 Hz, 1H), 6.50 (dd, J=16.3, 2.4 Hz, 1H), 4.85-4.71 (m, 1H), 4.10 (s, 2H), 3.31 (t, 4H), 2.88-2.73 (m, 2H), 2.73-2.63 (m, 3H), 2.53 (t, 12 J=5.1 Hz, 4H), 2.23 (d, J=7.2 Hz, 2H), 1.94 (qd, J=13.0, 5.1 Hz, 1H), 1.78-1.71 (m, 2H), 1.71-1.57 (m, 1H), 1.46 (s, 9H), 1.09 (qd, J=12.5, 4.3 Hz, 2H). LC/MS (ESI) m/z 532.4; [M+H]⁺ calcd for C₂₇H₃₉FN₅O₅⁺: 532.29.

Step 2: Synthesis of (R)—N-(2,6-dioxopiperidin-3-yl)-2-fluoro-4-(4-(piperidin-4-ylmethyl) piperazin-1-yl)benzamide hydrochloride (138-2). To a stirring solution of 138-1 (13 mg, 0.026 mmol) in DCM (0.5 mL) was added 4 N HCl in dioxane (0.5 mL) and stirred at room temperature for 2 hours. The reaction mixture was concentrated to afford 138-2 (13 mg, quantitative yield), which was directly used in the next step. LC/MS (ESI) m/z 432.3; [M+H]⁺ calcd for C₂₂H₃₁FN₅O₃⁺: 432.24.

Step 3: Synthesis of 4-(4-((1-(((R)-6-((4-(4-(((4-(((R)-4-(1,4-oxazepan-4-yl)-1-(phenylthio) butan-2-yl)amino)-3-((trifluoromethyl) sulfonyl)phenyl) sulfonyl) carbamoyl)phenyl) piperazin-1-yl)methyl)-4′-chloro-4-methyl-2,3,4,5-tetrahydro-[1,1′-biphenyl]-4-yl)methyl) piperidin-4-yl)methyl) piperazin-1-yl)-N-((R)-2,6-dioxopiperidin-3-yl)-2-fluorobenzamide (compound 138). Intermediate B (17 mg, 0.017 mmol) and NaBH(OAc)₃ (4.3 mg, 0.02 mmol) were added to a solution of 138-2 (13 mg, 0.027 mmol) and TEA (19 μL, 0.14 mmol) in DCM (1 mL), then stirred at room temperature overnight. The resulting mixture was washed with sat. aq. NH₄Cl and dried over Na₂SO₄, filtered and concentrated. The residue was purified by preparative TLC (DCM/MeOH) to afford compound #138 (6.21 mg, 26% yield). ¹H NMR (600 MHZ, Chloroform-d) δ 8.34 (d, J=2.2 Hz, 1H), 8.07 (s, 1H), 8.05-7.99 (m, 1H), 7.93 (t, J=9.2 Hz, 1H), 7.77 (d, J=8.5 Hz, 2H), 7.40 (dd, J=14.2, 5.8 Hz, 1H), 7.38-7.34 (m, 2H), 7.32-7.20 (m, 6H), 7.04-6.94 (m, 3H), 6.77-6.64 (m, 3H), 6.53-6.43 (m, 2H), 4.78 (dtd, J=12.7, 5.5, 1.8 Hz, 1H), 3.83 (s, 1H), 3.76 (t, J=6.1 Hz, 2H), 3.74-3.68 (m, 2H), 3.24 (d, J=29.7 Hz, 9H), 3.09 (dd, J=13.9, 5.0 Hz, 1H), 3.02 (dd, J=13.9, 7.0 Hz, 1H), 2.92-2.64 (m, 6H), 2.64-2.55 (m, 1H), 2.51 (t, J=5.2 Hz, 4H), 2.45-2.19 (m, 12H), 2.18-1.83 (m, 12H), 1.82-1.69 (m, 2H), 1.68-1.54 (m, 3H), 1.53-1.44 (m, 1H), 1.04 (s, 3H).). LC/MS (ESI) m/z 1417.7; [M+H]⁺ calcd for C₇₀H₈₆ClF₄N₁₀O₉S₃⁺: 1417.54.

### Example 39: Preparation of Compound #139

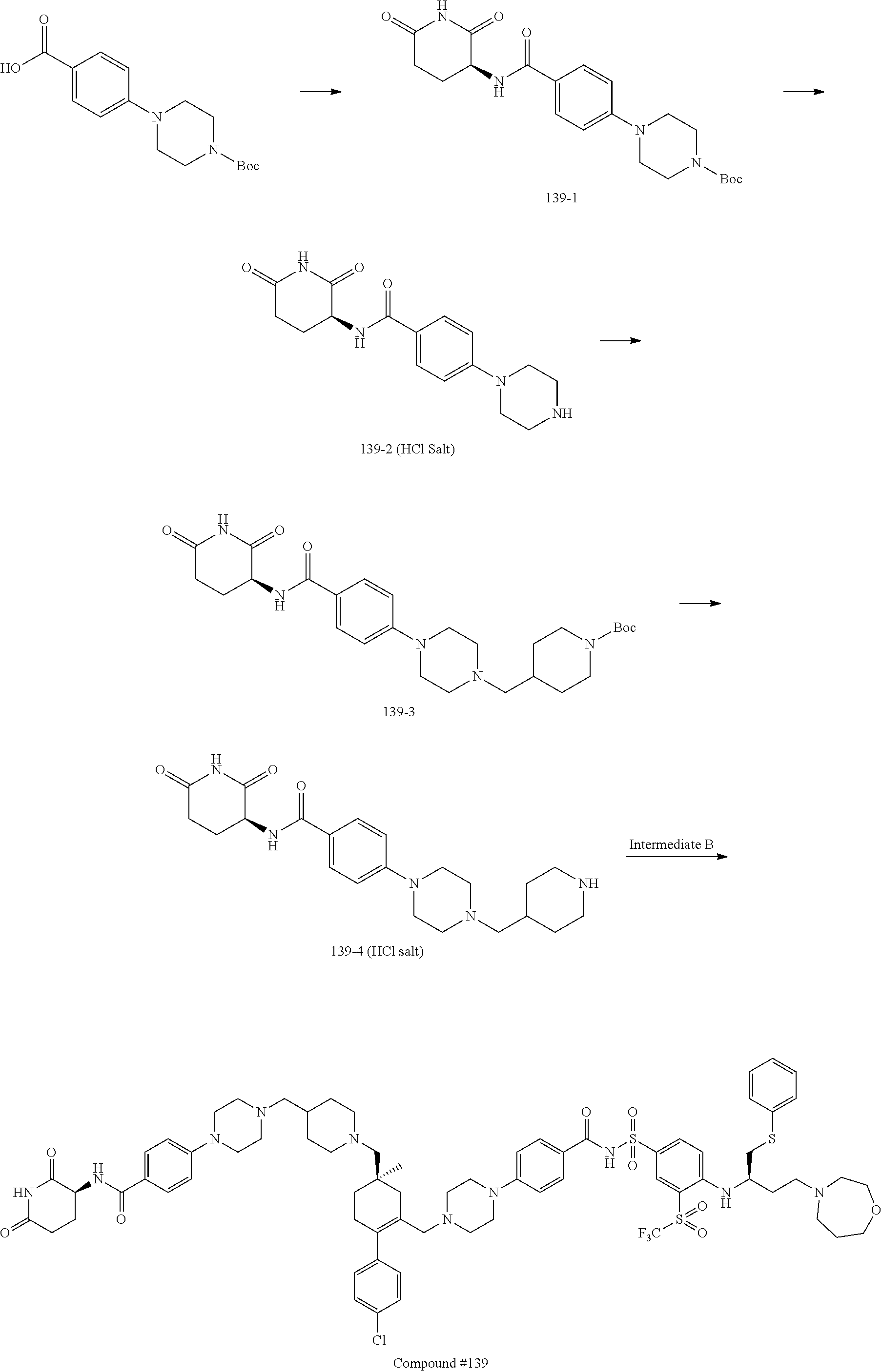 [SMILES: CC(C)(C)OC(=O)N1CCN(c2ccc(C(=O)O)cc2)CC1 ; CC(C)(C)OC(=O)N1CCN(c2ccc(C(=O)N[C@H]3CCC(=O)NC3=O)cc2)CC1 ; O=C1CC[C@H](NC(=O)c2ccc(N3CCNCC3)cc2)C(=O)N1 ; CC(C)(C)OC(=O)N1CCC(CN2CCN(c3ccc(C(=O)N[C@H]4CCC(=O)NC4=O)cc3)CC2)CC1 ; O=C1CC[C@H](NC(=O)c2ccc(N3CCN(CC4CCNCC4)CC3)cc2)C(=O)N1 ; C[C@@]1(CN2CCC(CN3CCN(c4ccc(C(=O)N[C@H]5CCC(=O)NC5=O)cc4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Step 1: Synthesis of tert-butyl(S)-4-(4-((2,6-dioxopiperidin-3-yl) carbamoyl)phenyl) piperazine-1-carboxylate (139-1). To a solution of 4-(4-(tert-butoxycarbonyl) piperazin-1-yl)benzoic acid (25 mg, 0.082 mmol) and HATU (40 mg, 0.11 mmol) in (1.0 mL) was added a solution of(S)-3-aminopiperidine-2,6-dione hydrochloride (15 mg, 0.091 mmol) and DIPEA (57 μL, 0.33 mmol) in DCM (1.0 mL). The resulting mixture was stirred at room temperature for 2 hours, then washed with water and sat. aq. NH₄Cl. The organic layers were dried over Na₂SO₄, filtered and concentrated. The residue was purified by preparative TLC (DCM/MeOH=10/1) to afford 140-1 (26 mg, 76% yield). ¹H NMR (600 MHZ, Chloroform-d) δ 7.95 (s, 1H), 7.80-7.76 (m, 2H), 6.96-6.89 (m, 2H), 6.86 (d, J=5.1 Hz, 1H), 4.76 (dt, J=12.5, 5.0 Hz, 1H), 3.61 (t, J=5.2 Hz, 4H), 3.31 (t, J=5.2 Hz, 4H), 2.92-2.75 (m, 3H), 1.97-1.86 (m, 1H), 1.51 (s, 9H). LC/MS (ESI) m/z 417.1; [M+H]⁺ calcd for C₂₁H₂₉N₄O₅⁺: 417.21.

Step 2: Synthesis of(S)—N-(2,6-dioxopiperidin-3-yl)-4-(piperazin-1-yl)benzamide hydrochloride (139-2). To a stirring solution of 139-1 (25 mg, 0.06 mmol) in DCM (0.5 mL) was added 4 N HCl in dioxane (0.5 mL) and stirred at room temperature for 2 hours. The reaction mixture was concentrated to afford 139-2 (23 mg, quantitative yield), which was directly used in the next step. LC/MS (ESI) m/z 317.2; [M+H]⁺ calcd for C₁₆H₂₁N₄O₃⁺: 317.16.

12 Step 3: Synthesis of tert-butyl(S)-4-((4-(4-((2,6-dioxopiperidin-3-yl) carbamoyl)phenyl) piperazin-1-yl)methyl) piperidine-1-carboxylate (139-3). tert-butyl 4-formylpiperidine-1-carboxylate (16 mg, 0.075 mmol) and NaBH(OAc)₃ (28 mg, 0.13 mmol) were added to a solution of 139-2 (23 mg, 0.063 mmol) and TEA (45 μL, 0.33 mmol) in DCM (1 mL), then stirred at room temperature overnight. The resulting mixture was washed with water and sat. aq. NH₄Cl. The organic layers were dried over Na₂SO₄, filtered and concentrated. The residue was purified by preparative TLC (DCM/MeOH=10/1) to afford 139-3 (12 mg, 39% yield in two steps). ¹H NMR (600 MHZ, Chloroform-d) δ 8.54 (s, 1H), 7.73 (dd, J=9.0, 2.1 Hz, 2H), 6.93 (d, J=5.5 Hz, 1H), 6.87 (d, J=8.9 Hz, 1H), 4.79-4.70 (m, 1H), 4.08 (s, 2H), 3.30 (t, J=4.8 Hz, 4H), 2.92-2.73 (m, 2H), 2.73-2.64 (m, 3H), 2.58 (t, J=5.0 Hz, 4H), 2.25 (d, J=7.0 Hz, 2H), 1.95-1.82 (m, 1H), 1.78-1.65 (m, 3H), 1.45 (s, 9H), 1.10 (qd, J=12.4, 4.3 Hz, 2H). LC/MS (ESI) m/z 514.2; [M+H]⁺ calcd for C₂₇H₄₀N₅O₅⁺: 514.3.

Step 4: Synthesis of(S)—N-(2,6-dioxopiperidin-3-yl)-4-(4-(piperidin-4-ylmethyl) piperazin-1-yl)benzamide hydrochloride (139-4). To a stirring solution of 139-3 (12 mg, 0.023 mmol) in DCM (0.5 mL) was added 4 N HCl in dioxane (0.5 mL) and stirred at room temperature for 2 hours. The reaction mixture was concentrated to afford 139-4 (10 mg, quantitative yield), which was directly used in the next step. LC/MS (ESI) m/z 414.2; [M+H]⁺ calcd for C₂₂H₃₂N₅O₃⁺: 414.25.

Step 5: Synthesis of N-((4-(((R)-4-(1,4-oxazepan-4-yl)-1-(phenylthio) butan-2-yl)amino)-3-((trifluoromethyl) sulfonyl)phenyl) sulfonyl)-4-(4-(((R)-4′-chloro-4-((4-((4-(4-(((S)-2,6-dioxopiperidin-3-yl) carbamoyl)phenyl) piperazin-1-yl)methyl) piperidin-1-yl)methyl)-4-methyl-3,4,5,6-tetrahydro-[1,1′-biphenyl]-2-yl)methyl) piperazin-1-yl)benzamide (compound #139). Intermediate B (15 mg, 0.015 mmol) and NaBH(OAc)₃ (4.0 mg, 0.019 mmol) were added to a solution of 139-4 (10 mg, 0.022 mmol) and TEA (17 μL, 0.12 mmol) in DCM (1 mL), then stirred at room temperature overnight. The resulting mixture was washed with sat. aq. NH₄Cl and dried over Na₂SO₄, filtered and concentrated. The residue was purified by preparative TLC (DCM/MeOH) to afford compound #139 (5.89 mg, 28% yield in two steps). ¹H NMR (600 MHZ, Chloroform-d) δ 8.33 (d, J=2.2 Hz, 1H), 8.02 (d, 1H), 7.79 (d, J=8.4 Hz, 2H), 7.75 (d, 2H), 7.39-7.34 (m, 2H), 7.32-7.20 (m, 5H), 7.05-6.92 (m, 4H), 6.85 (d, J=8.5 Hz, 2H), 6.73 (d, J=8.6 Hz, 2H), 6.47 (d, J=9.2 Hz, 1H), 4.81-4.68 (m, 1H), 3.82 (s, 1H), 3.76 (t, J=6.1 Hz, 2H), 3.73-3.67 (m, 2H), 3.22 (d, J=23.4 Hz, 9H), 3.09 (dd, J=13.8, 4.9 Hz, 1H), 3.01 (dd, J=13.9, 6.9 Hz, 1H), 2.90-2.70 (m, 5H), 2.71-2.65 (m, 1H), 2.60 (m, 1H), 2.53 (s, 4H), 2.49-2.19 (m, 12H), 2.20-1.83 (m, 12H), 1.80-1.68 (m, 2H), 1.66-1.53 (m, 3H), 1.52-1.40 (m, 2H), 1.02 (s, 3H).). LC/MS (ESI) m/z 1399.7; [M+H]⁺ calcd for C₇₀H₈₇ClF₃N₁₀O₉S₃⁺: 1399.55.

### Example 40: Preparation of Compound #140

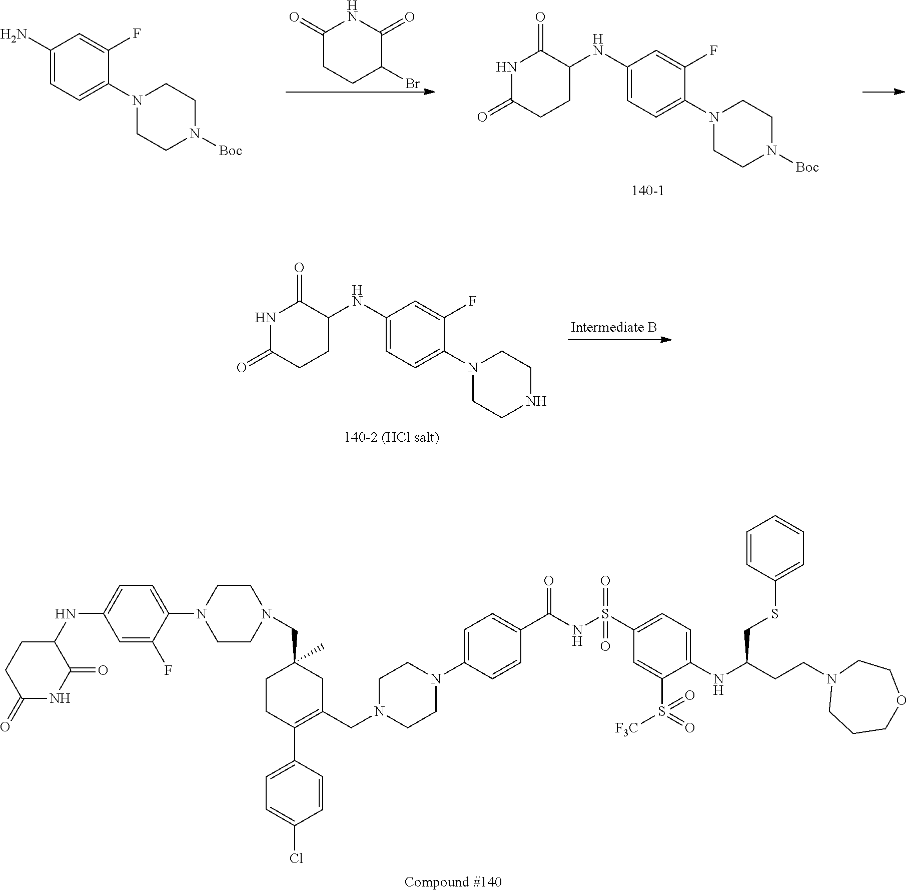 [SMILES: CC(C)(C)OC(=O)N1CCN(c2ccc(N)cc2F)CC1 ; O=C1CCC(Br)C(=O)N1 ; CC(C)(C)OC(=O)N1CCN(c2ccc(NC3CCC(=O)NC3=O)cc2F)CC1 ; O=C1CCC(Nc2ccc(N3CCNCC3)c(F)c2)C(=O)N1 ; C[C@@]1(CN2CCN(c3ccc(NC4CCC(=O)NC4=O)cc3F)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

Step 1: Synthesis of tert-butyl 4-(4-((2,6-dioxopiperidin-3-yl)amino)-2-fluorophenyl) piperazine-1-carboxylate (140-1). A mixture of tert-butyl 4-(4-amino-2-fluorophenyl) piperazine-1-carboxylate (295 mg, 1.0 mmol), 3-bromopiperidine-2,6-dione (288 mg, 1.5 mmol) and DIPEA (695 μL, 4.0 mmol) in DMSO (6 mL) was stirred at 70° C. overnight. The resulting mixture was cooled to room temperature and diluted with EtOAc, then washed with water and sat. aq. NH₄Cl. The organic layers were dried over Na₂SO₄, filtered and concentrated. The residue was purified by flash column chromatography (20% to 80% of EtOAc in hexanes) to afford 140-1 (290 mg, 71% yield) as a light-green solid. ¹H NMR (600 MHZ, Chloroform-d) δ 8.00 (s, 1H), 6.87 (t, J=8.9 Hz, 1H), 6.49-6.39 (m, 2H), 4.64 (d, J=3.8 Hz, 1H), 4.03 (dt, J=12.5, 4.4 Hz, 1H), 3.60 (t, J=5.0 Hz, 4H), 3.00-2.86 (m, 5H), 2.77 (ddd, J=18.2, 13.5, 5.2 Hz, 1H), 2.58-2.51 (m, 1H), 1.99-1.87 (m, 1H), 1.50 (s, 9H). LC/MS (ESI) m/z 407.1; [M+H]⁺ calcd for C₂₀H₂₈N₄O₄⁺: 407.21.

Step 2: Synthesis of 3-((3-fluoro-4-(piperazin-1-yl)phenyl)amino) piperidine-2,6-dione hydrochloride (140-2). To a solution of 137-1 (100 mg, 0.246 mmol) in DCM (4 mL) was added a solution of 4 N HCl in dioxane (4 mL) and stirred at room temperature for 2 hours. The resulting mixture was concentrated to afford 140-2 (112 mg, quantitative yield), which was directly used in the next step. LC/MS (ESI) m/z 307.2; [M+H]⁺ calcd for C₁₅H₂₀N₄O₂⁺: 307.16.

Step 3: Synthesis of N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-(4-((2,6-dioxopiperidin-3-yl)amino)-2-fluorophenyl) piperazin-1-yl)methyl)-4-methyl-3,4,5,6-tetrahydro-[1,1′-biphenyl]-2-yl)methyl) piperazin-1-yl)benzamide (compound #140). Intermediate B (15 mg, 0.015 mmol) and NaBH(OAc)₃ (3 mg, 0.023 mmol) were added to a solution of 140-2 (10.3 mg, 0.03 mmol) and TEA (13 μL, 0.09 mmol) in DCM (1 mL), then stirred at room temperature overnight. The resulting mixture was washed with sat. aq. NH₄Cl. The organic layers were dried over Na₂SO₄, filtered and concentrated. The residue was purified by preparative TLC (DCM/MeOH) to afford compound #140 (4.91 mg, 19% yield) as an off-white solid. ¹H NMR (599 MHz, Chloroform-d) δ 8.41-8.27 (m, 2H), 8.07 (dt, J=9.2, 2.4 Hz, 1H), 7.78-7.70 (m, 2H), 7.41-7.35 (m, 2H), 7.34-7.21 (m, 6H), 7.09 (d, J=8.6 Hz, 1H), 7.04-6.99 (m, 2H), 6.86 (td, J=9.1, 2.4 Hz, 1H), 6.77 (dd, J=9.1, 3.2 Hz, 2H), 6.58 (d, J=9.3 Hz, 1H), 6.47-6.39 (m, 2H), 4.57 (s, 1H), 4.02 (dt, J=12.4, 4.3 Hz, 1H), 3.95-3.85 (m, 1H), 3.82-3.69 (m, 4H), 3.26 (t, J=5.6 Hz, 4H), 3.11 (dd, J=13.9, 5.0 Hz, 1H), 3.07-2.95 (m, 5H), 2.92-2.58 (m, 12H), 2.57-2.21 (m, 11H), 2.16-2.06 (m, 1H), 2.01-1.87 (m, 4H), 1.83-1.72 (m, 1H), 1.71-1.60 (m, 1H), 1.51-1.42 (m, 1H), 0.99 (d, J=2.0 Hz, 3H). LC/MS (ESI) m/z 1292.6; [M+H]⁺ calcd for C₆₃H₇₅ClF₄N₉O₈S₃⁺: 1292.45.

### Example 41: Preparation of Compound #141

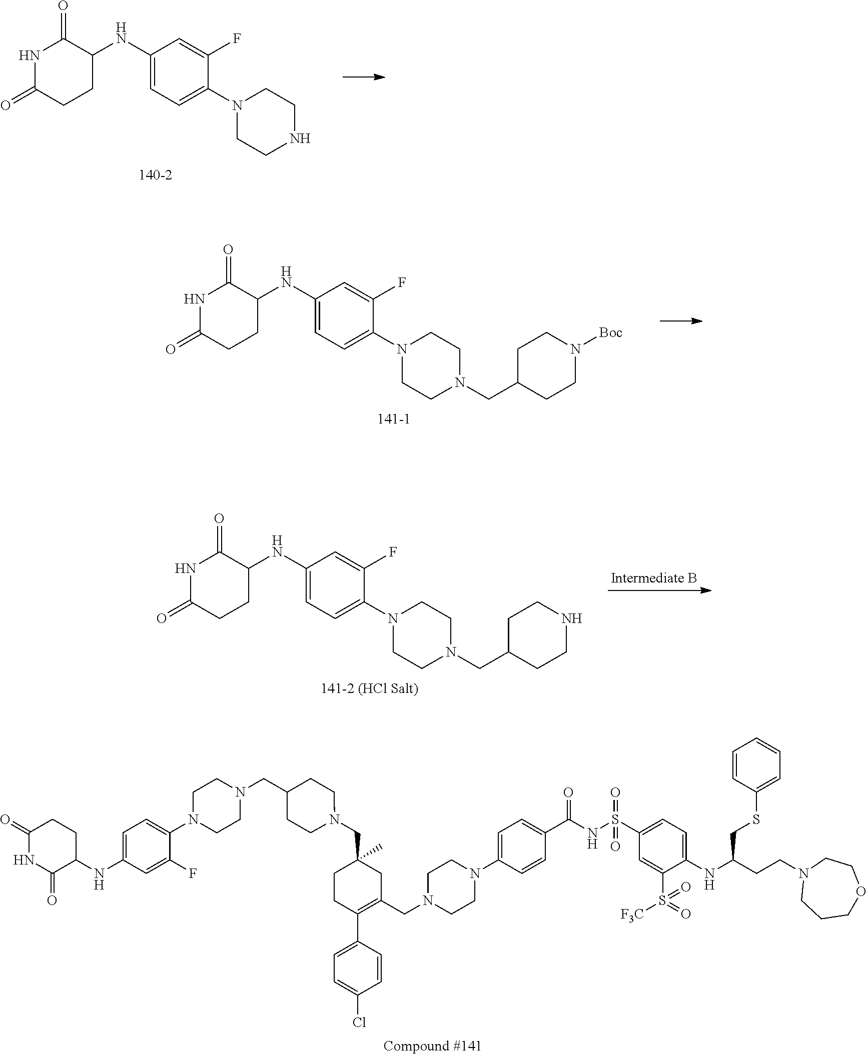 [SMILES: O=C1CCC(Nc2ccc(N3CCNCC3)c(F)c2)C(=O)N1 ; CC(C)(C)OC(=O)N1CCC(CN2CCN(c3ccc(NC4CCC(=O)NC4=O)cc3F)CC2)CC1 ; O=C1CCC(Nc2ccc(N3CCN(CC4CCNCC4)CC3)c(F)c2)C(=O)N1 ; C[C@@]1(CN2CCC(CN3CCN(c4ccc(NC5CCC(=O)NC5=O)cc4F)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1]

### Compound #141 was Prepared by Following General Procedure C:

tert-butyl 4-((4-(4-((2,6-dioxopiperidin-3-yl)amino)-2-fluorophenyl) piperazin-1-yl)methyl) piperidine-1-carboxylate (141-1) (115 mg, 70% yield). ¹H NMR (600 MHZ, Chloroform-d) δ 7.97 (s, 1H), 6.89 (t, J=8.9 Hz, 1H), 6.49-6.39 (m, 2H), 4.61 (d, J=3.7 Hz, 1H), 4.21-4.05 (m, 2H), 4.02 (dt, J=12.5, 4.4 Hz, 1H), 3.02 (t, J=5.3 Hz, 4H), 2.89 (ddd, J=18.0, 4.5, 2.7 Hz, 1H), 2.81-2.66 (m, 2H), 2.64-2.51 (m, 5H), 2.26 (d, J=7.2 Hz, 2H), 1.92 (qd, J=13.5, 4.5 Hz, 1H), 1.77 (d, J=13.2 Hz, 2H), 1.72-1.65 (m, 1H), 1.48 (s, 9H), 1.11 (qd, J=12.4, 4.3 Hz, 2H). LC/MS (ESI) m/z 504.3; [M+H]⁺ calcd for C₂₆H₃₉N₅O₄⁺: 504.30.

3-((3-fluoro-4-(4-(piperidin-4-ylmethyl) piperazin-1-yl)phenyl)amino) piperidine-2,6-dione hydrochloride (141-2) (20 mg, quantitative yield). LC/MS (ESI) m/z 404.2; [M+H]⁺ calcd for C₂₁H₃₁N₅O₂⁺: 404.25.

N-((4-(((R)-4-(1,4-oxazepan-4-yl)-1-(phenylthio) butan-2-yl)amino)-3-((trifluoromethyl) sulfonyl)phenyl) sulfonyl)-4-(4-(((4R)-4′-chloro-4-((4-((4-(4-((2,6-dioxopiperidin-3-yl)amino)-2-fluorophenyl) piperazin-1-yl)methyl) piperidin-1-yl)methyl)-4-methyl-3,4,5,6-tetrahydro-[1,1′-biphenyl]-2-yl)methyl) piperazin-1-yl)benzamide (compound #141) (3.89 mg, 14% yield). ¹H NMR (600 MHZ, Chloroform-d) δ 8.36 (d, J=1.8 Hz, 1H), 8.12 (s, 1H), 8.05-7.99 (m, 1H), 7.84 (d, J=8.4 Hz, 2H), 7.41-7.36 (m, 2H), 7.34-7.22 (m, 6H), 7.00 (d, J=8.3 Hz, 2H), 6.96 (d, J=8.5 Hz, 1H), 6.86 (t, J=9.0 Hz, 1H), 6.76 (d, J=8.6 Hz, 2H), 6.49-6.39 (m, 3H), 4.62 (s, 1H), 4.02 (dd, J=12.5, 4.7 Hz, 1H), 3.88-3.69 (m, 5H), 3.29-3.16 (m, 4H), 3.16-3.07 (m, 2H), 3.07-2.96 (m, 6H), 2.92-2.72 (m, 8H), 2.73-2.57 (m, 7H), 2.55-2.42 (m, 4H), 2.41-2.18 (m, 12H), 2.18-2.01 (m, 1H), 2.00-1.85 (m, 3H), 1.83-1.56 (m, 4H), 1.55-1.41 (m, 1H), 1.03 (s, 3H). LC/MS (ESI) m/z 1389.6; [M+H]⁺ calcd for C₆₉H₈₆ClF₄N₁₀O₈S₃⁺: 1389.54.

### Example 42. Cell Viability Assay

Acute lymphoblastic leukemia cells (MOLT-4 and RS4;11) were incubated with increasing concentrations of Bcl-xL/Bcl-2 degraders for 48 h. Cell viability was measured by tetrazolium-based MTS assay. The IC₅₀ values of individual agents were calculated with GraphPad Prism and presented in Table 1.

TABLE 1
Anti-cancer activity of compounds of Formula (I)

| Compound | Anti-Cancer Activity EC₅₀ (48 hr)^{a} | | Senolytic Activity EC₅₀ (72 hr)^{a} | | | |
|---|---|---|---|---|---|---|
| # | MOLT-4 | RS4; 11 | HUVEC IR-SC | HUVEC NC | WI-38 IR-SC | WI-38 NC |
| 1 | +++++ | +++++ | ND | ND | ++++ | + |
| 2 | +++++ | +++++ | ND | ND | ++++ | + |
| 3 | +++++ | +++++ | ND | ND | ND | + |
| 4 | +++++ | +++++ | ND | ND | ++++ | + |
| 5 | +++++ | +++++ | ND | ND | +++ | + |
| 6 | +++++ | +++++ | ND | ND | ++++ | + |
| 7 | +++++ | +++++ | ND | ND | ++++ | + |
| 8 | +++++ | +++++ | ND | ND | ++++ | + |
| 9 | +++++ | +++++ | ND | ND | +++ | + |
| 10 | +++++ | +++++ | ND | ND | +++ | + |
| 11 | +++++ | +++++ | +++++ | ND | ++++ | + |
| 12 | +++++ | +++++ | ND | ND | +++ | + |
| 13 | +++++ | +++++ | ND | ND | ++++ | + |
| 14 | +++++ | +++++ | ND | ND | ++ | + |
| 15 | +++++ | +++++ | ND | ND | ++ | + |
| 16 | +++++ | +++++ | ND | ND | +++ | + |
| 17 | +++++ | +++++ | ND | ND | ND | + |
| 18 | +++++ | +++++ | ND | ND | +++ | + |
| 19 | ++++ | +++ | ND | ND | ND | ND |
| 20 | ++++ | +++ | ND | ND | ND | ND |
| 21 | +++ | ++ | ND | ND | ND | ND |
| 22 | +++ | +++ | ND | ND | ND | ND |
| 23 | ++ | +++ | ND | ND | ND | ND |
| 24 | ++++ | +++++ | ND | ND | ND | + |
| 26 | +++++ | ++++ | ND | ND | ND | + |
| 29 | ++++ | +++++ | ND | ND | ND | + |
| 37 | +++++ | ++++ | ND | ND | ND | + |
| 38 | ++++ | ++++ | ND | ND | ND | + |
| 40 | +++++ | +++++ | ND | ND | ND | + |
| 44 | +++++ | +++++ | +++++ | ++ | ++++ | + |
| 45 | ++++ | ++++ | ND | ND | ND | ++ |
| 46 | +++++ | +++++ | ND | ND | ND | + |
| 50 | +++++ | +++++ | ND | ND | ND | ++ |
| 65 | +++++ | +++++ | ND | ND | ND | + |
| 127 | +++++ | +++++ | ND | ND | ND | + |
| 128 | +++++ | +++++ | +++++ | ++ | ++++ | ND |
| 129 | +++++ | +++++ | ND | ND | ND | + |
| 130 | ++++ | ++++ | ND | ND | ND | + |
| 131 | ++++ | ++++ | ND | ND | ND | + |
| 132 | +++++ | ++++ | ND | ND | ND | + |
| 133 | +++++ | ++++ | +++++ | ND | +++ | ND |
| 134 | +++++ | +++++ | +++++ | ++ | ++++ | ND |
| 135 | +++++ | +++++ | +++++ | ++ | +++ | ND |
| 136 | +++++ | +++++ | ND | ND | ND | ND |
| 137 | ++++ | ++++ | ND | ND | ND | ND |
| 138 | ++++ | ++++ | ND | ND | ND | ND |
| 139 | ++++ | ++++ | ND | ND | ND | ND |
| 140 | ++++ | +++ | ND | ND | ND | ND |
| 141 | ++++ | ++++ | +++++ | ND | ++++ | + |

^{a}+++++: <1 nM;
++++: 1-10 nM;
+++: 10-100 nM;
++: 100-10 μM;
+: >10 μM;
ND: Not determined.

### Example 43: Cell Viability Assay of Senescent Cells

To induce senescence in WI-38 and HUVEC, cells were subjected to x-ray irradiation at 20 Gray using X-RAD 320 biological irradiator. Following between 10 to 15 days after irradiation, senescence was induced in cells, and irradiation-induced senescent cells (IR-SC) were then used for cell viability and Western blotting tests.

WI-38 and HUVEC cells were incubated with increasing concentrations of degraders for 72 h. Cell viability was measured using PrestoBlue HS cell viability reagent. The IC₅₀ values of individual compounds were calculated with GraphPad Prism and presented in Table 1.

### Example 44: Protein Degradation Assays in Jurkat, Hela and WI-38 Cells

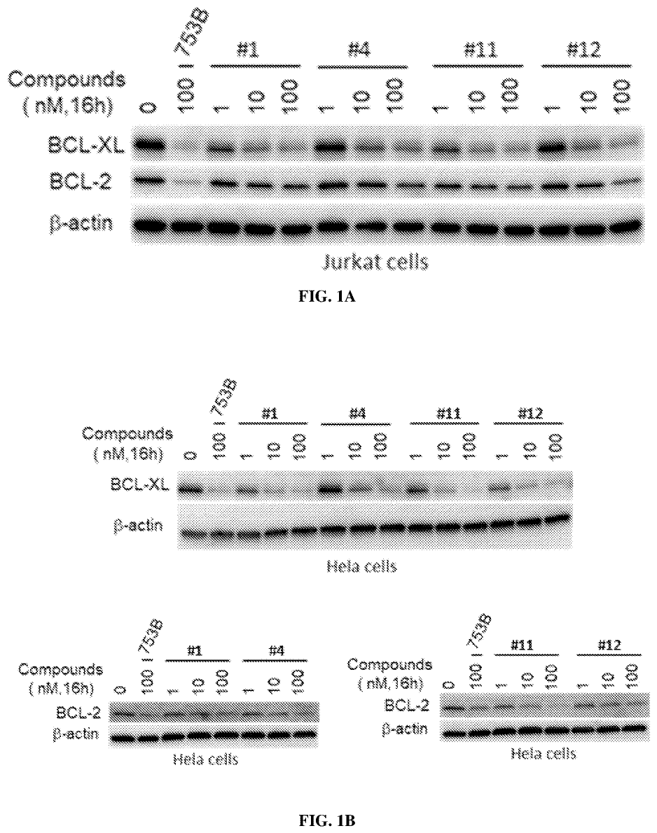

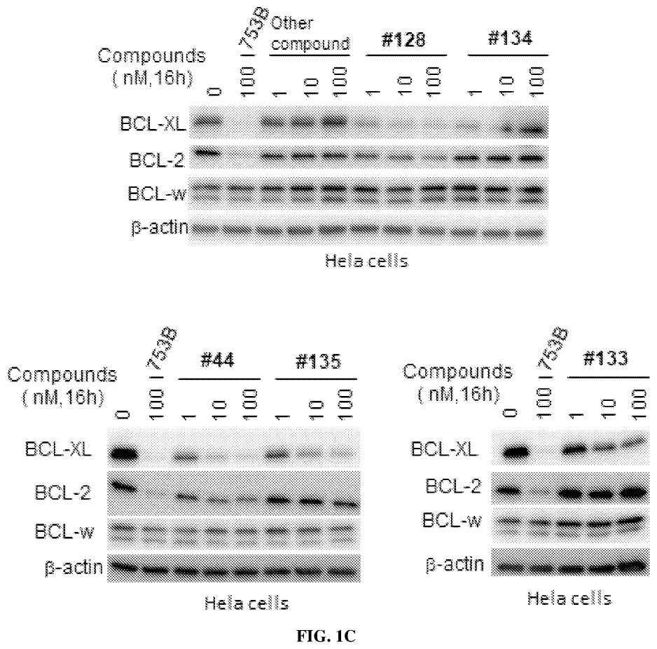

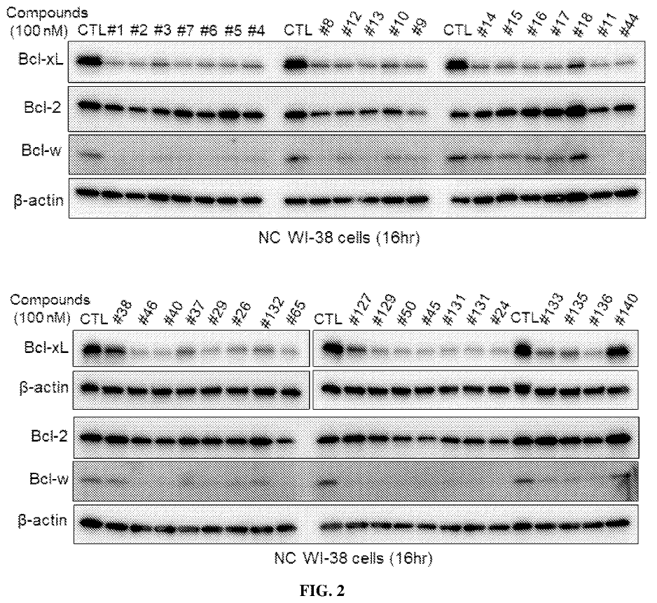

Jurkat and Hela cells were incubated with increasing concentrations of test compounds for 16 h. The cells were harvested and lysed in RIPA lysis buffer supplemented with protease and phosphatase inhibitor cocktails. An equal amount of protein (20 μg/lane) was resolved on a pre-cast 4-20% SDS-PAGE gel. Proteins were subsequently transferred to NOVEX PVDF membranes by electrophoresis. The membranes were blocked in blocking buffer (5% non-fat dry milk in TBS-T), and incubated with primary antibodies (at optimized concentrations) overnight at 4° C. After three washings in TBS-T, the membranes were incubated with an appropriate FIRP-conjugated secondary antibody for 1 h at room temperature. After extensive washing for three times, the proteins of interest were detected with ECL western blotting detection reagents and recorded with autoradiography (Pierce Biotech, Rockford, IL, USA). The primary antibodies for Bcl-xL (Cat #2762), Bcl-2 (Cat #2872), Bcl-w (Cat #2724) and β-actin (Cat #4970) were purchased from Cell Signaling Technology. The relative band intensity was measured using ImageJ software and normalized to β-actin. Representative data are presented in FIGS. 1A, 1B, 1C, and 2.

753B or 753b is a compound of the formula:

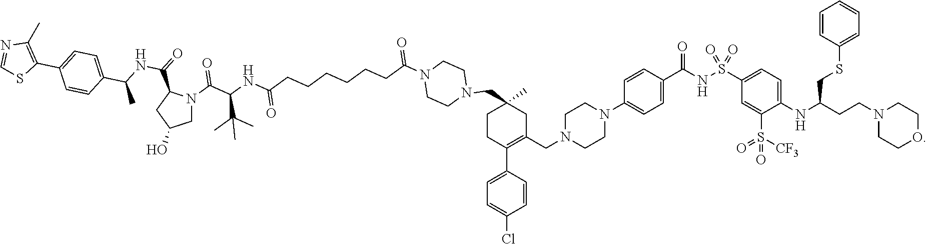 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

### INCORPORATION BY REFERENCE

The contents of all references (including literature references, issued patents, published patent applications, and co-pending patent applications) cited throughout this application are hereby expressly incorporated herein in their entireties by reference.

### Equivalents and Scope

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising,” “including,” and “containing,” and all other tenses thereof, are intended to be open and permits the inclusion of additional possibilities (e.g., elements or steps). Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

What is claimed is:

1. A compound of Formula (I), or a pharmaceutically acceptable salt, solvate, hydrate, polymorph, co-crystal, tautomer, stereoisomer, isotopically labeled compound, or prodrug thereof: 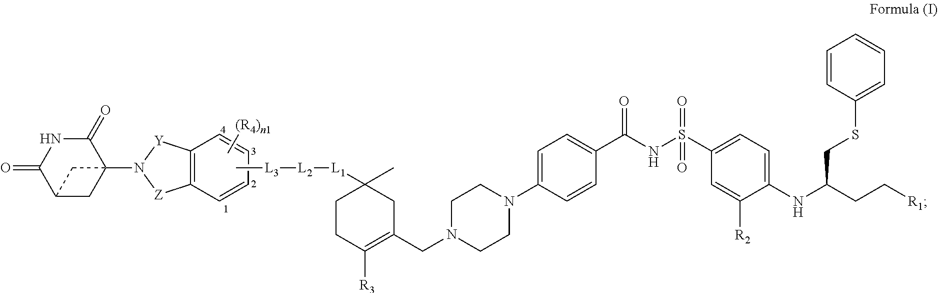
wherein:
R₁ is —O(substituted or unsubstituted alkyl) or —N(R^{a})₂, wherein each instance of R^{a} is independently substituted or unsubstituted alkyl or substituted or unsubstituted carbocyclyl, or two instances of R^{a} are joined with the nitrogen atom to which they are attached to form substituted or unsubstituted heterocyclyl;
R₂ is SO₂CF₃ or NO₂;
R₃ is 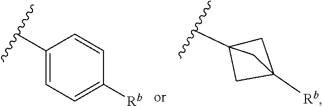
wherein R^{b} is halogen or substituted or unsubstituted alkyl;
each instance of R⁴ is independently F, Cl, —CN, —CFH₂, —CF₂H, —CF₃, or —OCH₃;
n1 is 0, 1, 2, or 3;
Z is a bond, CH₂, or CO;
Y is CO;
or 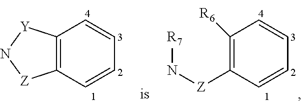
wherein R⁶ is F, Cl, CN, CFH₂, —CF₂H, —CF₃, —OCH₃, or H; and R⁷ is H or substituted or unsubstituted alkyl;
L₁ is substituted or unsubstituted, C₁₋₁₁ alkylene, substituted or unsubstituted, C₂₋₁₁ alkenylene, substituted or unsubstituted, C₂₋₁₁ alkynylene, or substituted or unsubstituted heterocyclylene, optionally wherein one or more backbone carbon atoms in the substituted or unsubstituted, C₁₋₁₁ alkylene, substituted or unsubstituted, C₂₋₁₁ alkenylene, or substituted or unsubstituted, C₂₋₁₁ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene;
each instance of R₅ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
L₂ is substituted or unsubstituted, C₁₋₁₂ alkylene, substituted or unsubstituted, C₂₋₁₂ alkenylene, substituted or unsubstituted, C₂₋₁₂ alkynylene, substituted or unsubstituted heterocyclylene, or substituted or unsubstituted carbocyclylene, optionally wherein one or more backbone carbon atoms in the substituted or unsubstituted, C₁₋₁₂ alkylene, substituted or unsubstituted, C₂₋₁₂ alkenylene, or substituted or unsubstituted, C₂₋₁₂ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene; and
L₃ is a bond, substituted or unsubstituted, C₁₋₁₃ alkylene, substituted or unsubstituted, C₂₋₁₃ alkenylene, substituted or unsubstituted, C₂₋₁₃ alkynylene, or substituted or unsubstituted heterocyclylene, optionally wherein one or more backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene.

2. The compound of claim 1, or a pharmaceutically acceptable salt, solvate, hydrate, polymorph, co-crystal, tautomer, stereoisomer, isotopically labeled compound, or prodrug thereof, wherein:
R₆ is F, Cl, —CN, —CFH₂, —CF₂H, —CF₃, or —OCH₃, and
L₃ is substituted or unsubstituted, C₁₋₁₃ alkylene, substituted or unsubstituted, C₂₋₁₃ alkenylene, substituted or unsubstituted, C₂₋₁₃ alkynylene, or substituted or unsubstituted heterocyclylene, optionally wherein one or more backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, ═N—, —N═, —S—, —S(═O)—, —S(═O)₂—, —C(═O)—, substituted or unsubstituted heterocyclylene, substituted or unsubstituted heteroarylene, substituted or unsubstituted carbocyclylene, or substituted or unsubstituted arylene.

3. The compound of claim 1 or 2, or a pharmaceutically acceptable salt, solvate, hydrate, polymorph, co-crystal, tautomer, stereoisomer, isotopically labeled compound, or prodrug thereof, wherein R₁ is —O(substituted or unsubstituted alkyl).

4. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —O(alkyl substituted at least with substituted or unsubstituted heterocyclyl, wherein at least one heteroatom in the heterocyclic ring system is nitrogen).

5. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —N(R^{a})₂, wherein each instance of R^{a} is independently substituted or unsubstituted alkyl or substituted or unsubstituted carbocyclyl.

6. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —N(R^{a})₂, wherein two instances of R^{a} are joined with the nitrogen atom to which they are attached to form substituted or unsubstituted heterocyclyl.

7. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —N(R^{a})₂, wherein two instances of R^{a} are joined with the nitrogen atom to which they are attached to form substituted or unsubstituted, monocyclic heterocyclyl.

8. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —N(R^{a})₂, wherein two instances of R^{a} are joined with the nitrogen atom to which they are attached to form substituted or unsubstituted, fused bicyclic heterocyclyl.

9. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —N(R^{a})₂, wherein two instances of R^{a} are joined with the nitrogen atom to which they are attached to form substituted or unsubstituted, bridged bicyclic heterocyclyl.

10. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is —N(R^{a})₂, wherein two instances of R^{a} are joined with the nitrogen atom to which they are attached to form substituted or unsubstituted, spiro bicyclic heterocyclyl.

11. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is 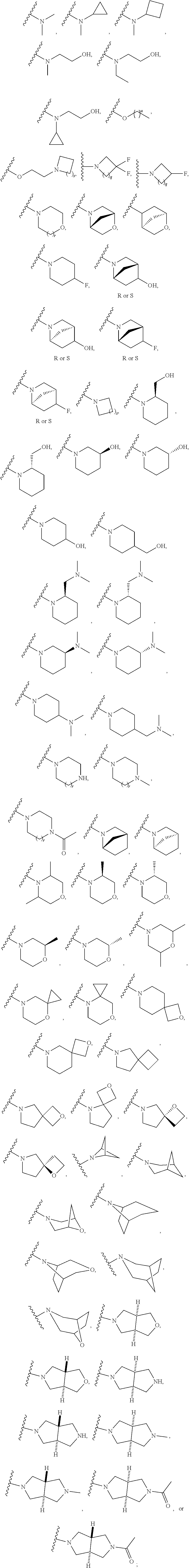
wherein:
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2;
each s is independently 1, 2, 3, or 4;
each t is independently 1, 2, 3, 4, or 5; and
each u is independently 0, 1, 2, 3, 4, or 5.

12. The compound of any one of claims 1-2, or a pharmaceutically acceptable salt, solvate, hydrate, polymorph, co-crystal, tautomer, stereoisomer, isotopically labeled compound, or prodrug thereof, wherein R₁ is of the formula:

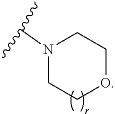

13. The compound of any one of claims 1-12, or a pharmaceutically acceptable salt, solvate, hydrate, polymorph, co-crystal, tautomer, stereoisomer, isotopically labeled compound, or prodrug thereof, wherein R₂ is NO₂.

14. The compound of any one of claims 1-12, or a pharmaceutically acceptable salt, solvate, hydrate, polymorph, co-crystal, tautomer, stereoisomer, isotopically labeled compound, or prodrug thereof, wherein R₂ is SO₂CF₃.

15. The compound of any one of claims 1-14, or a pharmaceutically acceptable salt, solvate, hydrate, polymorph, co-crystal, tautomer, stereoisomer, isotopically labeled compound, or prodrug thereof, wherein R³ is

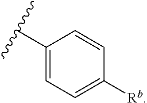

16. The compound of claim 15, or a pharmaceutically acceptable salt, solvate, hydrate, polymorph, co-crystal, tautomer, stereoisomer, isotopically labeled compound, or prodrug thereof, wherein R³ is

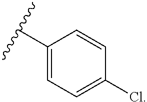 [SMILES: *c1ccc(Cl)cc1]

17. The compound of any one of claims 1-14, or a pharmaceutically acceptable salt, solvate, hydrate, polymorph, co-crystal, tautomer, stereoisomer, isotopically labeled compound, or prodrug thereof, wherein R³ is

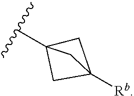

18. The compound of any one of claims 1-15 and 17, or a pharmaceutically acceptable salt, solvate, hydrate, polymorph, co-crystal, tautomer, stereoisomer, isotopically labeled compound, or prodrug thereof, wherein R^{b} is halogen.

19. The compound of claim 18, or a pharmaceutically acceptable salt, solvate, hydrate, polymorph, co-crystal, tautomer, stereoisomer, isotopically labeled compound, or prodrug thereof, wherein R^{b} is Cl.

20. The compound of any one of claims 1-15 and 17, or a pharmaceutically acceptable salt, solvate, hydrate, polymorph, co-crystal, tautomer, stereoisomer, isotopically labeled compound, or prodrug thereof, wherein R^{b} is substituted or unsubstituted alkyl.

21. The compound of claim 20, or a pharmaceutically acceptable salt, solvate, hydrate, polymorph, co-crystal, tautomer, stereoisomer, isotopically labeled compound, or prodrug thereof, wherein R^{b} is alkyl substituted only with one or more instances of fluoro.

22. The compound of any one of claims 1-14, or a pharmaceutically acceptable salt, solvate, hydrate, polymorph, co-crystal, tautomer, stereoisomer, isotopically labeled compound, or
prodrug thereof, wherein R³ is

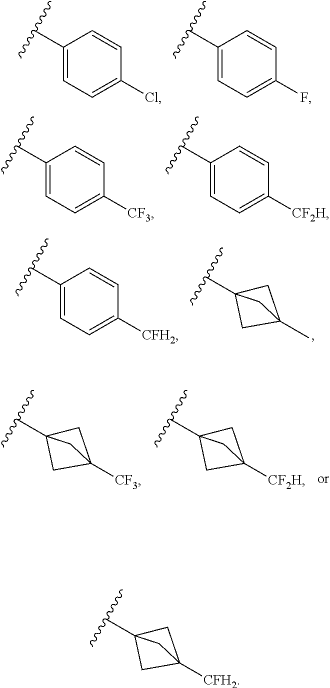

23. The compound of any one of claims 1-22, or a pharmaceutically acceptable salt, solvate, hydrate, polymorph, co-crystal, tautomer, stereoisomer, isotopically labeled compound, or prodrug thereof, wherein n1 is 0.

24. The compound of any one of claims 1-22, or a pharmaceutically acceptable salt, solvate, hydrate, polymorph, co-crystal, tautomer, stereoisomer, isotopically labeled compound, or prodrug thereof, wherein n1 is 1.

25. The compound of any one of claims 1-22 and 24, or a pharmaceutically acceptable salt, solvate, hydrate, polymorph, co-crystal, tautomer, stereoisomer, isotopically labeled compound, or prodrug thereof, wherein at least one instance of R₄ is F.

26. The compound of any one of claims 1-25, or a pharmaceutically acceptable salt, solvate, hydrate, polymorph, co-crystal, tautomer, stereoisomer, isotopically labeled compound, or prodrug thereof, wherein Z is a bond.

27. The compound of any one of claims 1-25, or a pharmaceutically acceptable salt, solvate, hydrate, polymorph, co-crystal, tautomer, stereoisomer, isotopically labeled compound, or prodrug thereof, wherein Z is CH₂.

28. The compound of any one of claims 1-25, or a pharmaceutically acceptable salt, solvate, hydrate, polymorph, co-crystal, tautomer, stereoisomer, isotopically labeled compound, or prodrug thereof, wherein Z is CO.

29. The compound of any one of claims 1-28, or a pharmaceutically acceptable salt, solvate, hydrate, polymorph, co-crystal, tautomer, stereoisomer, isotopically labeled compound, or prodrug thereof, wherein Y is CO.

30. The compound of any one of claims 1-25, or a pharmaceutically acceptable salt, solvate, hydrate, polymorph, co-crystal, tautomer, stereoisomer, isotopically labeled compound, or prodrug thereof, wherein

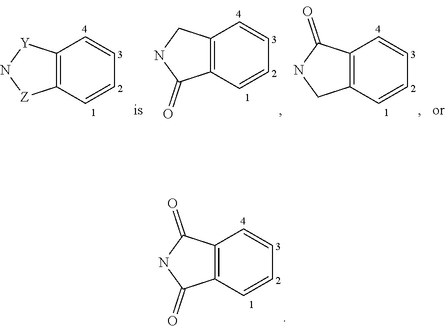

31. The compound of any one of claims 1-28, or a pharmaceutically acceptable salt, solvate, hydrate, polymorph, co-crystal, tautomer, stereoisomer, isotopically labeled compound, or prodrug thereof, wherein

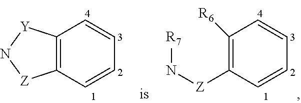

32. The compound of claim 31, or a pharmaceutically acceptable salt, solvate, hydrate, polymorph, co-crystal, tautomer, stereoisomer, isotopically labeled compound, or prodrug thereof, wherein

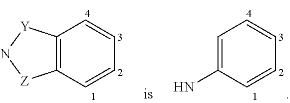

33. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is substituted or unsubstituted, C₁₋₁₁ alkylene.

34. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is substituted or unsubstituted, C₁₋₁₁ alkylene, wherein one or more backbone carbon atoms in the substituted or unsubstituted, C₁₋₁₁ alkylene are independently replaced with —O—, —NR₅—, —S(═O)—, —S(═O)₂—, or —C(═O)—.

35. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is substituted or unsubstituted, monocyclic heterocyclylene, substituted or unsubstituted, bicyclic heterocyclylene, or substituted or unsubstituted, monocyclic heteroarylene.

36. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is -(substituted or unsubstituted, monocyclic heterocyclylene)-(substituted or unsubstituted, C₁₋₂ alkylene)-, -(substituted or unsubstituted, bicyclic heterocyclylene)-(substituted or unsubstituted, C₁₋₂ alkylene)-, -(substituted or unsubstituted, monocyclic heteroarylene)-(substituted or unsubstituted, C₁₋₂ alkylene)-, -(substituted or unsubstituted, C₁₋₂ alkylene)-(substituted or unsubstituted, monocyclic heterocyclylene)-, —(substituted or unsubstituted, C₁₋₂ alkylene)-(substituted or unsubstituted, bicyclic heterocyclylene)-, or -(substituted or unsubstituted, C₁₋₂ alkylene)-(substituted or unsubstituted, monocyclic heteroarylene)-, optionally wherein one or two backbone carbon atoms in the substituted or unsubstituted, C₁₋₂ alkylene are independently replaced with —O—, —NR₅—, —S(═O)—, —S(═O)₂—, or —C(═O)—.

37. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is -(substituted or unsubstituted, C₁₋₂ alkylene)-(substituted or unsubstituted, monocyclic heterocyclylene)-(substituted or unsubstituted, C₁₋₂ alkylene)-, —(substituted or unsubstituted, C₁₋₂ alkylene)-(substituted or unsubstituted, bicyclic heterocyclylene)-(substituted or unsubstituted, C₁₋₂ alkylene)-, or -(substituted or unsubstituted, C₁₋₂ alkylene)-(substituted or unsubstituted, monocyclic heteroarylene)-(substituted or unsubstituted, C₁₋₂ alkylene)-, optionally wherein one or two backbone carbon atoms in each instance of the substituted or unsubstituted, C₁₋₂ alkylene are independently replaced with —O—, —NR₅—, —S(═O)—, —S(═O)₂—, or —C(═O)—.

38. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is -(substituted or unsubstituted, monocyclic heterocyclylene)₂-(substituted or unsubstituted, C₁ alkylene)-, -(substituted or unsubstituted, C₁ alkylene)-(substituted or unsubstituted, monocyclic heterocyclylene)₂-, -(substituted or unsubstituted, C₁ alkylene)-(substituted or unsubstituted, monocyclic heterocyclylene)₂-(substituted or unsubstituted, C₁ alkylene)-, -(substituted or unsubstituted, monocyclic heterocyclylene)-(substituted or unsubstituted, monocyclic carbocyclylene)-(substituted or unsubstituted, C₁ alkylene)-, -(substituted or unsubstituted, monocyclic carbocyclylene)-(substituted or unsubstituted, monocyclic heterocyclylene)-(substituted or unsubstituted, C₁ alkylene)-, —(substituted or unsubstituted, C₁ alkylene)-(substituted or unsubstituted, monocyclic heterocyclylene)-(substituted or unsubstituted, monocyclic carbocyclylene)-, or -(substituted or unsubstituted, C₁ alkylene)-(substituted or unsubstituted, monocyclic carbocyclylene)-(substituted or unsubstituted, monocyclic heterocyclylene)-, optionally wherein the backbone carbon atom in each instance of the substituted or unsubstituted, C₁ alkylene is independently replaced with —O—, —NR₅—, —S(═O)—, —S(═O)₂—, or —C(═O)—.

39. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is 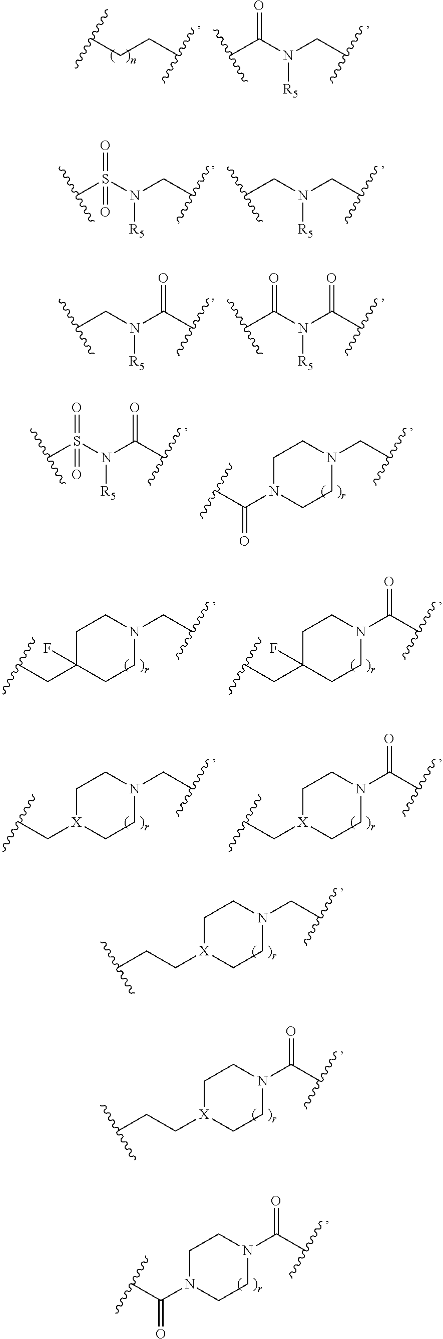 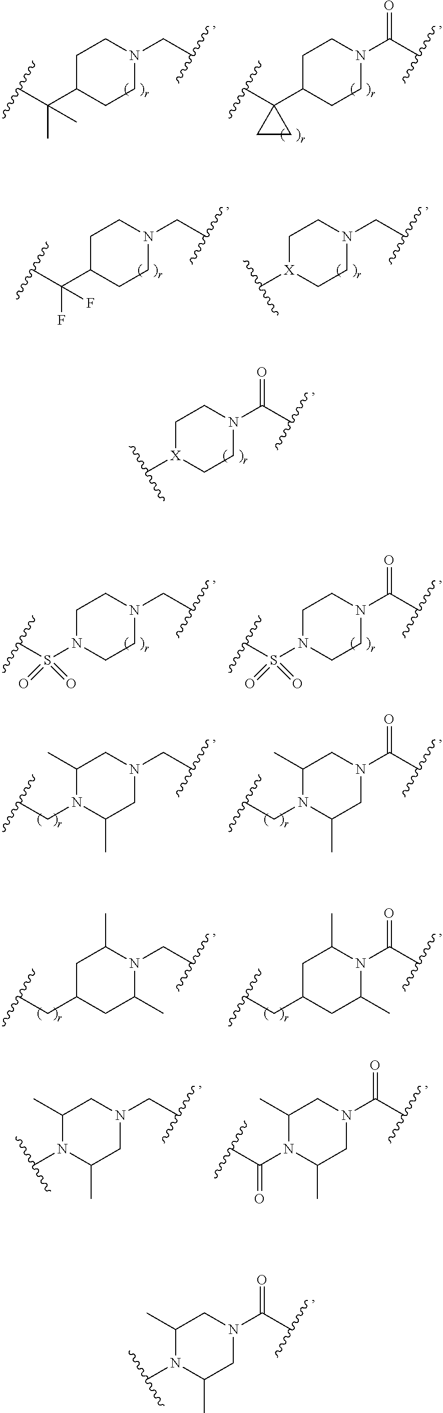 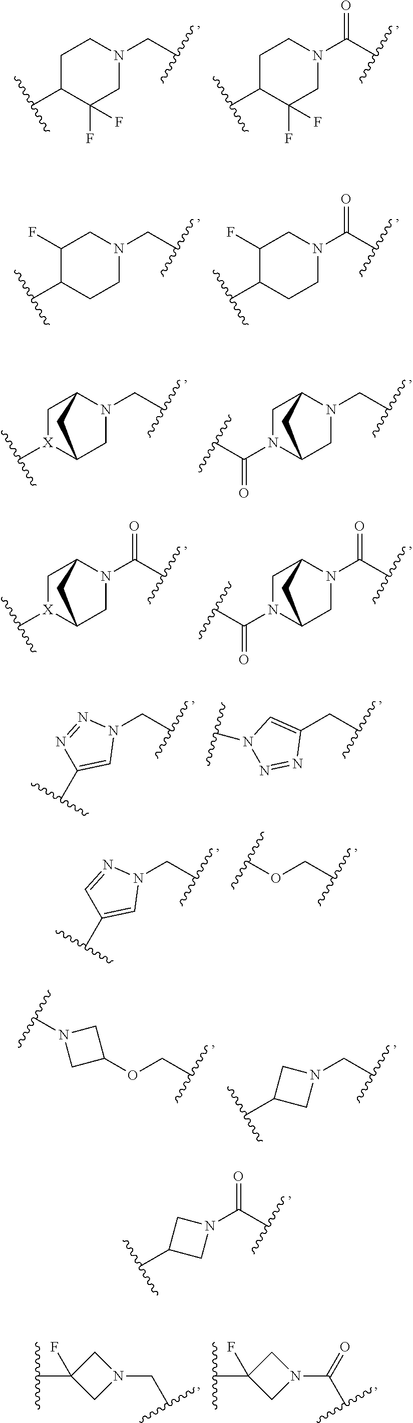 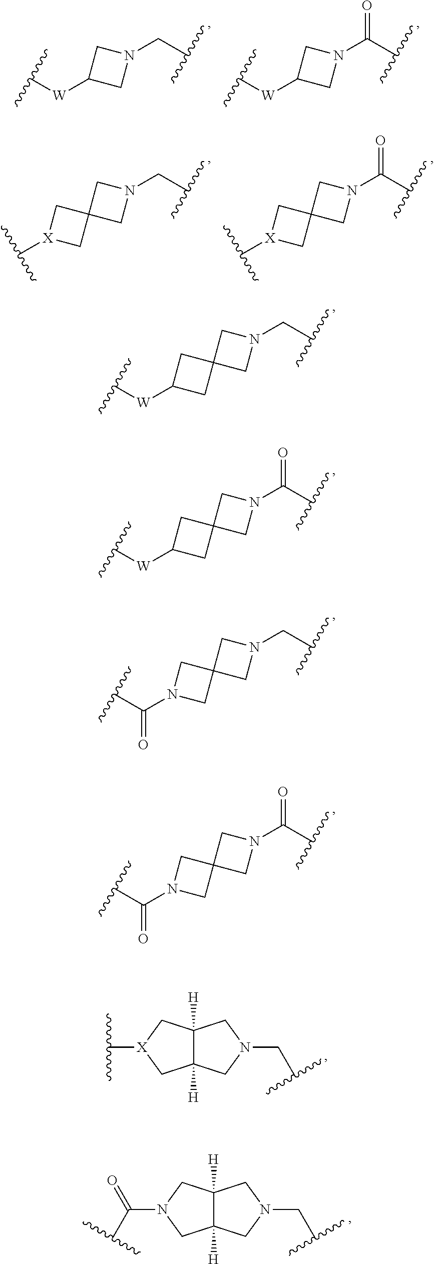 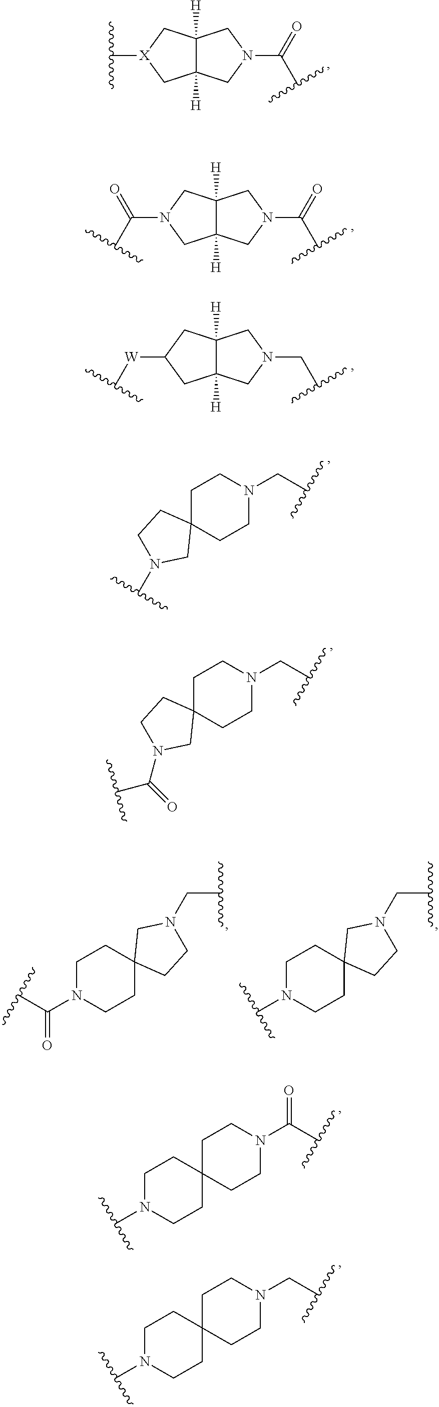 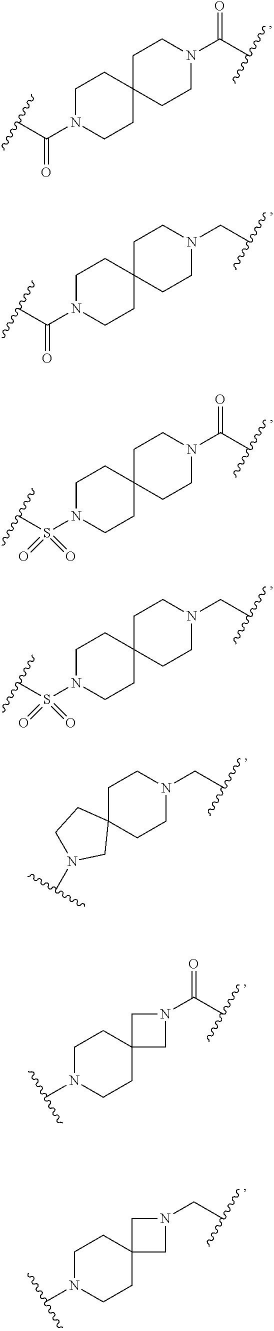 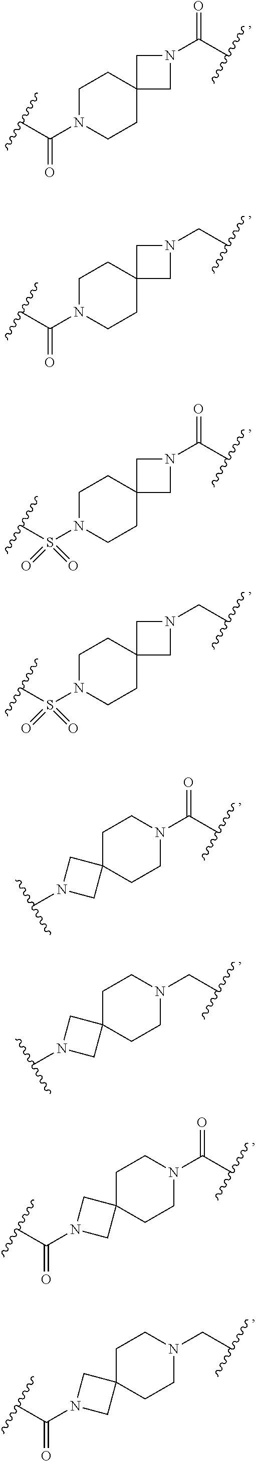 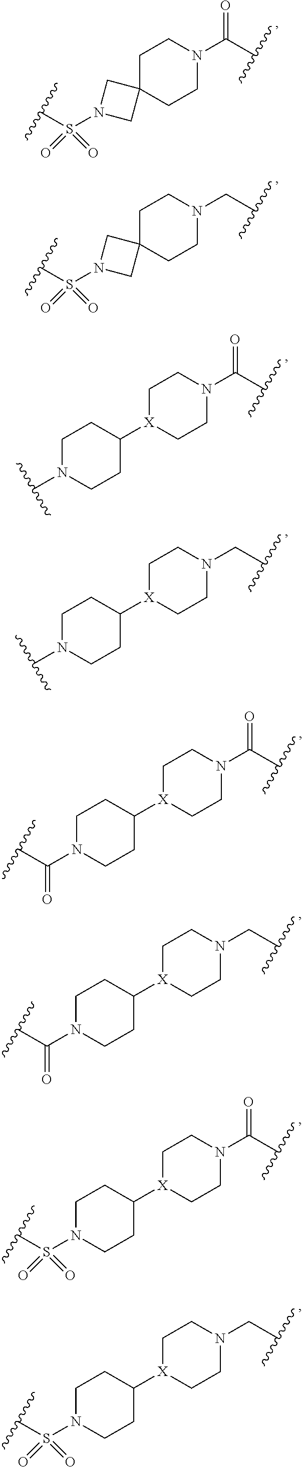 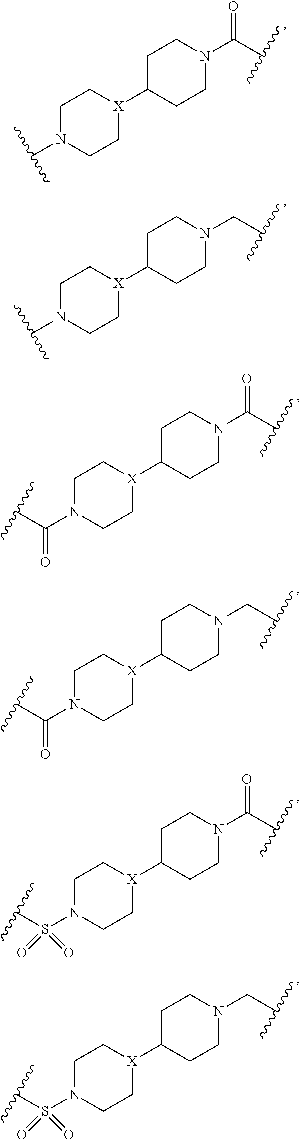 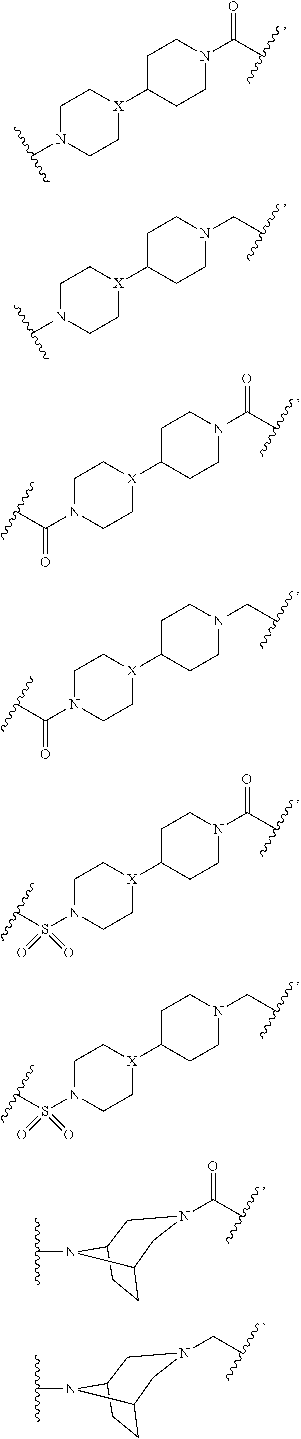 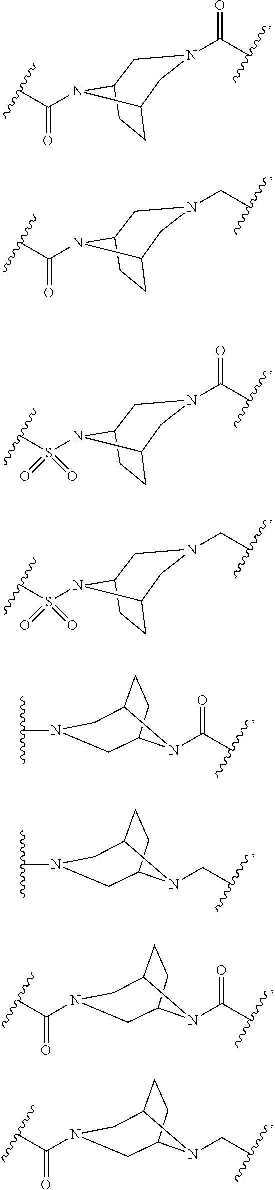 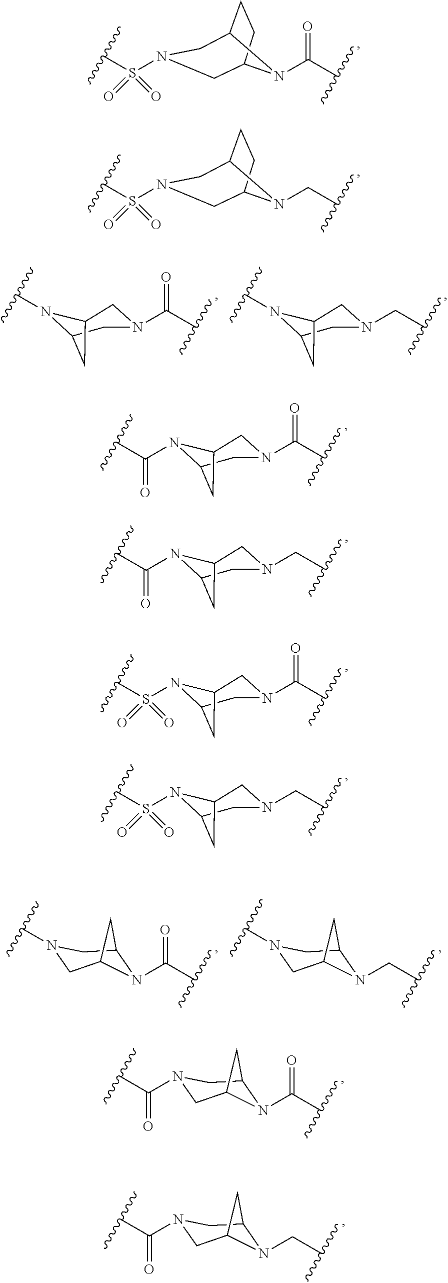 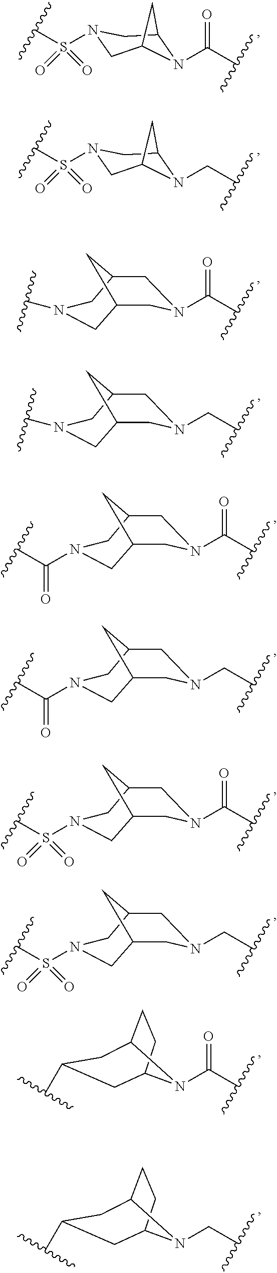 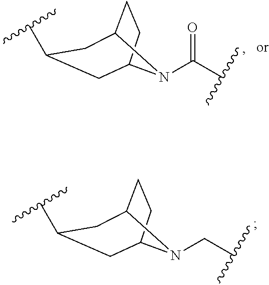
wherein:
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2;
each s is independently 1, 2, 3, or 4;
each t is independently 1, 2, 3, 4, or 5; and
each u is independently 0, 1, 2, 3, 4, or 5.

40. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is

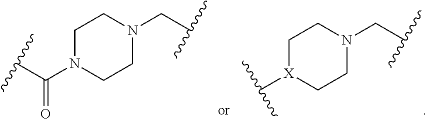

41. The compound of any one of claims 1-32, or a pharmaceutically acceptable salt, solvate, hydrate, polymorph, co-crystal, tautomer, stereoisomer, isotopically labeled compound, or prodrug thereof, wherein L₁ is —CH₂—.

42. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is substituted or unsubstituted, C₁₋₁₂ alkylene, substituted or unsubstituted, C₂₋₁₂ alkenylene, or substituted or unsubstituted, C₂₋₁₂ alkynylene.

43. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is substituted or unsubstituted, C₁₋₁₂ alkylene, substituted or unsubstituted, C₂₋₁₂ alkenylene, or substituted or unsubstituted, C₂₋₁₂ alkynylene, wherein one or more backbone carbon atoms in the substituted or unsubstituted, C₁₋₁₂ alkylene, substituted or unsubstituted, C₂₋₁₂ alkenylene, or substituted or unsubstituted, C₂₋₁₂ alkynylene are independently replaced with —O—, —NR₅—, —S(═O)—, —S(═O)₂—, or —C(═O)—.

44. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is substituted or unsubstituted heterocyclylene.

45. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is substituted or unsubstituted, monocyclic heterocyclylene.

46. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is -(substituted or unsubstituted, monocyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, bicyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, monocyclic heteroarylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, monocyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, —(substituted or unsubstituted, bicyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heterocyclylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic heterocyclylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heteroarylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic carbocyclylene)-, or -(substituted or unsubstituted, C₁₋₈ alkylene, substituted or unsubstituted, C₂₋₈ alkenylene, or substituted or unsubstituted, C₂₋₈ alkynylene) (substituted or unsubstituted, bicyclic carbocyclylene)-, optionally wherein one or more backbone carbon atoms in the substituted or unsubstituted, C₁₋₈ alkylene, substituted or unsubstituted, C₂₋₈ alkenylene, or substituted or unsubstituted, C₂₋₈ alkynylene are independently replaced with —O—, —NR₅—, —S(═O)—, —S(═O)₂—, or —C(═O)—.

47. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heterocyclylene) (substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic heterocyclylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic heteroarylene)-(substituted or unsubstituted, C₁₋₈ alkylene, substituted or unsubstituted, C₂₋₈ alkenylene, or substituted or unsubstituted, C₂₋₈ alkynylene)-, -(substituted or unsubstituted, C₁₋₈ alkylene, substituted or unsubstituted, C₂₋₈ alkenylene, or substituted or unsubstituted, C₂₋₈ alkynylene)-(substituted or unsubstituted, monocyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, or (substituted or unsubstituted, C₁₋₈ alkylene, substituted or unsubstituted, C₂₋₈ alkenylene, or substituted or unsubstituted, C₂₋₈ alkynylene)-(substituted or unsubstituted, bicyclic carbocyclylene)-(substituted or unsubstituted, C₁₋₈ alkylene, substituted or unsubstituted, C₂₋₈ alkenylene, or substituted or unsubstituted, C₂₋₈ alkynylene)-, optionally wherein one or more backbone carbon atoms in each instance of the substituted or unsubstituted, C₁₋₈ alkylene, substituted or unsubstituted, C₂₋₈ alkenylene, or substituted or unsubstituted, C₂₋₈ alkynylene are independently replaced with —O—, —NR₅—, —S(═O)—, —S(═O)₂—, or —C(═O)—.

48. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is -(substituted or unsubstituted, monocyclic heterocyclylene)₂-(substituted or unsubstituted, C₁₋₂ alkylene)-, -(substituted or unsubstituted, C₁₋₂ alkylene)-(substituted or unsubstituted, monocyclic heterocyclylene)₂-, or -(substituted or unsubstituted, C₁₋₂ alkylene)-(substituted or unsubstituted, monocyclic heterocyclylene)₂-(substituted or unsubstituted, C₁₋₂ alkylene)-, optionally wherein one or more of the backbone carbon atoms in each instance of the substituted or unsubstituted, C₁₋₂ alkylene are independently replaced with —O—, —NR₅—, —S(═O)—, —S(═O)₂—, or —C(═O)—.

49. The compound of any one of claims 1-41, or a pharmaceutically acceptable salt, solvate, hydrate, polymorph, co-crystal, tautomer, stereoisomer, isotopically labeled compound, or prodrug thereof, wherein L₂ is 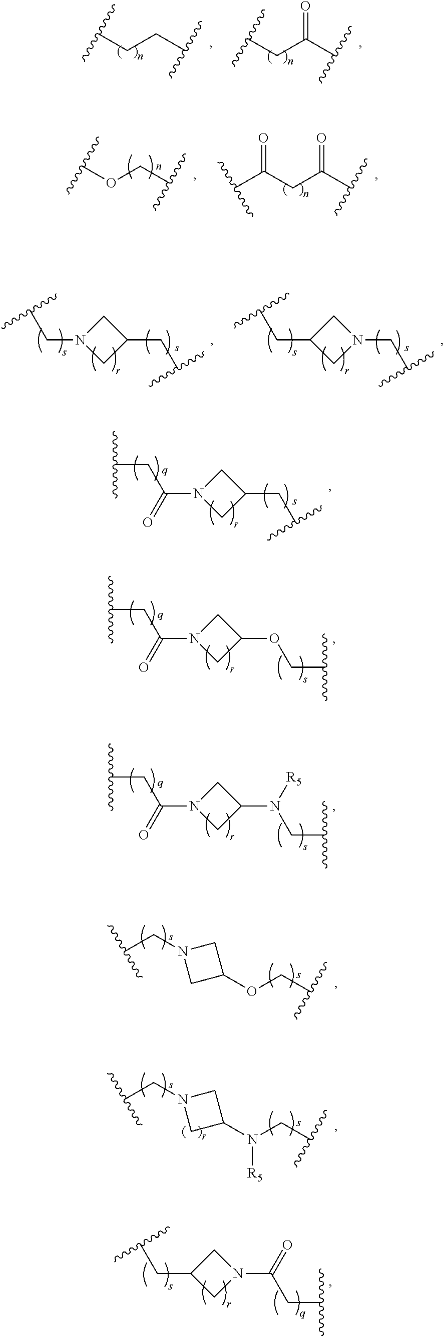 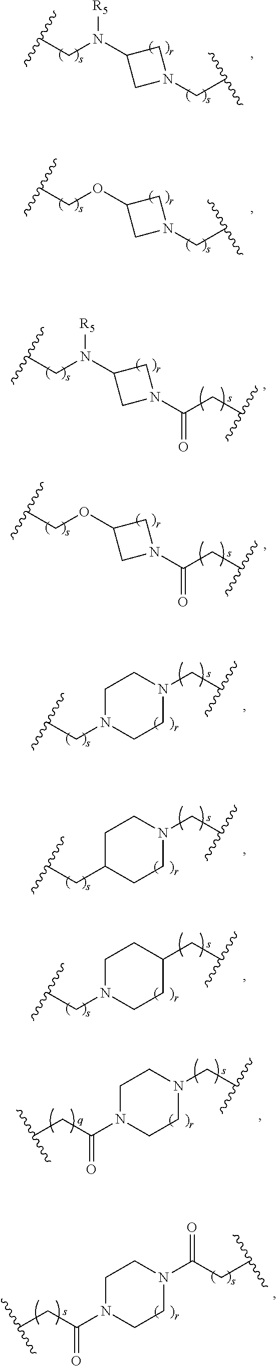 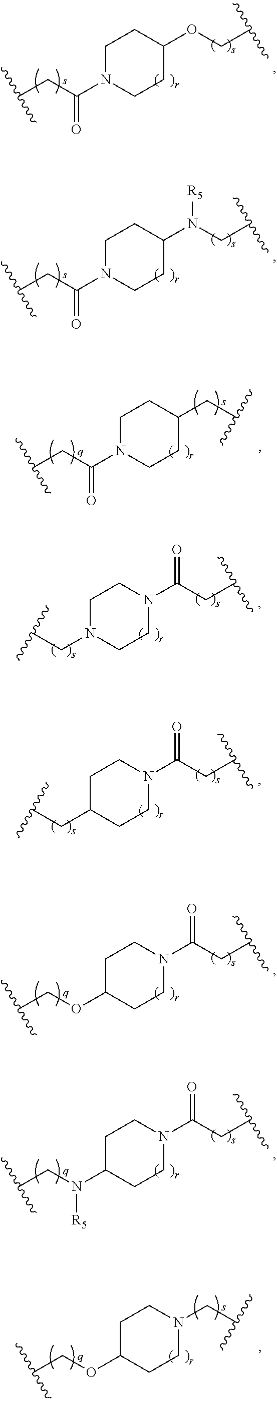 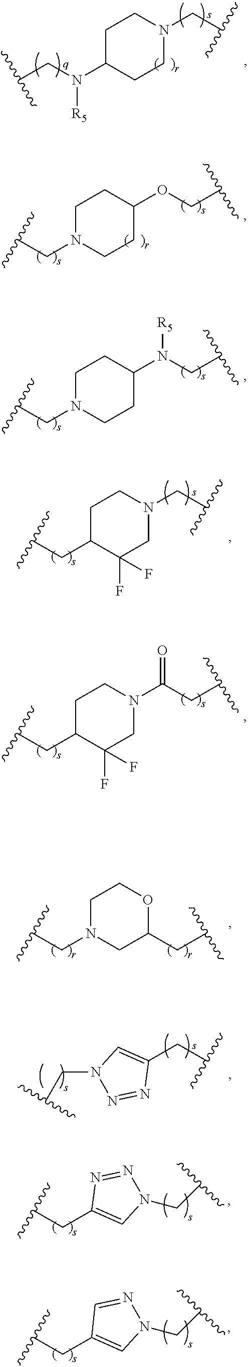 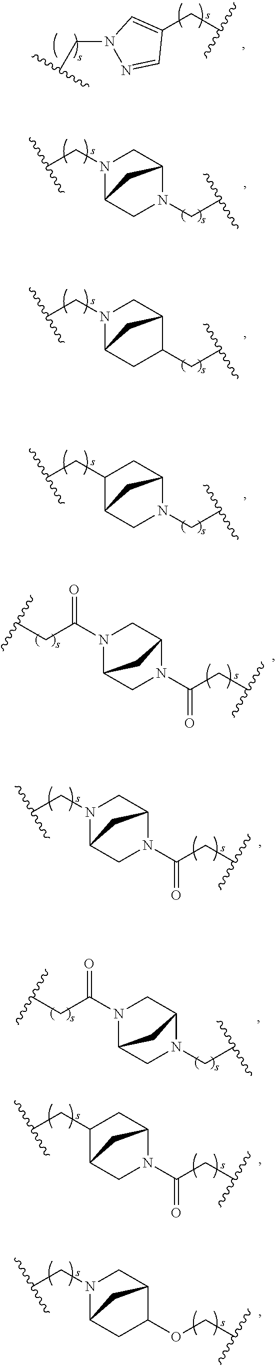 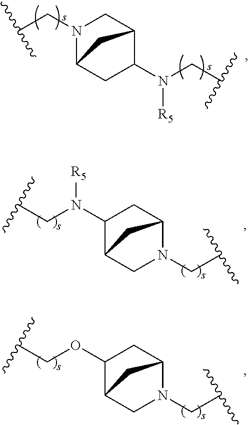 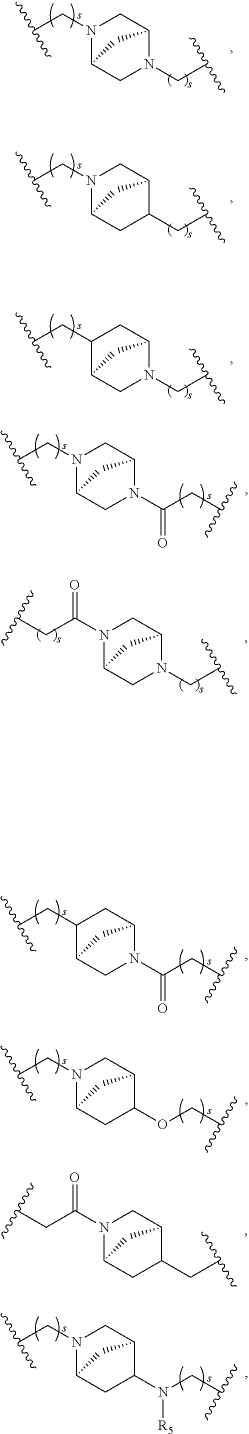 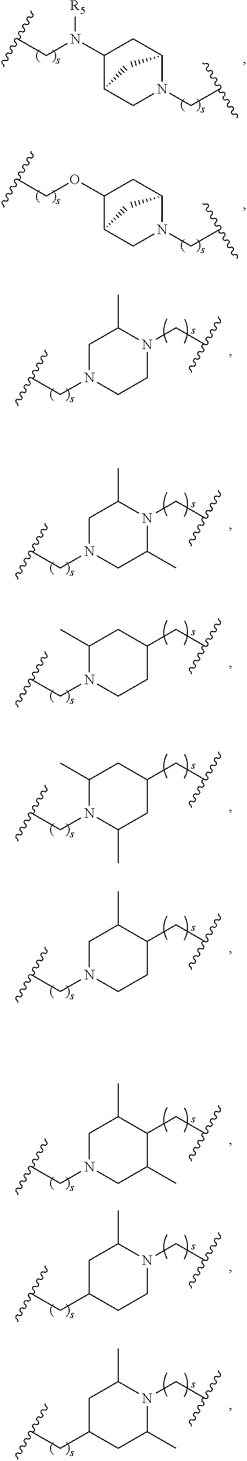 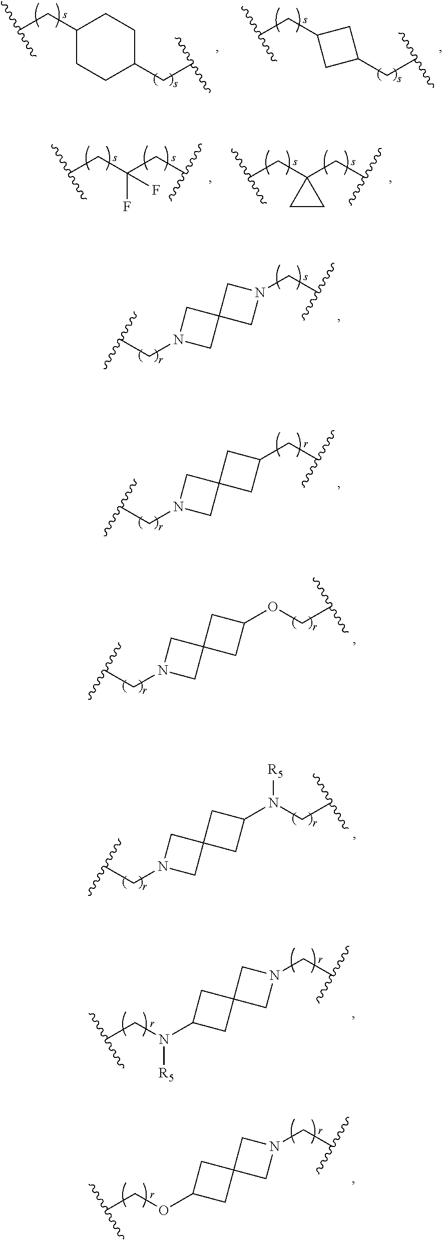 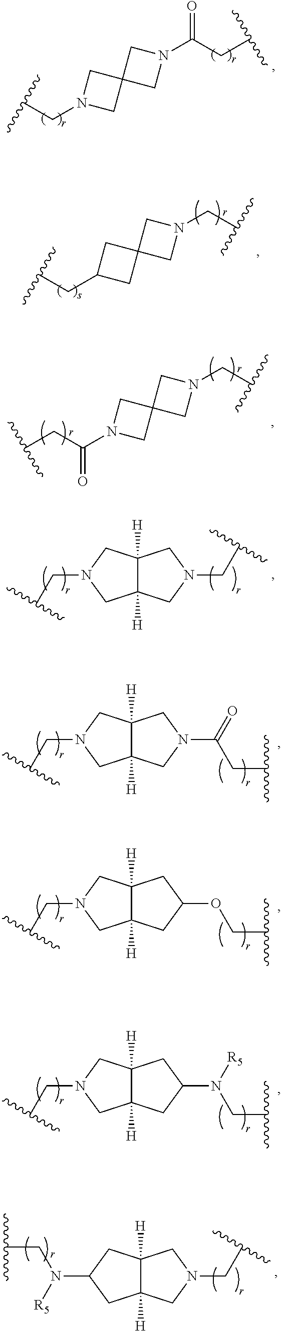 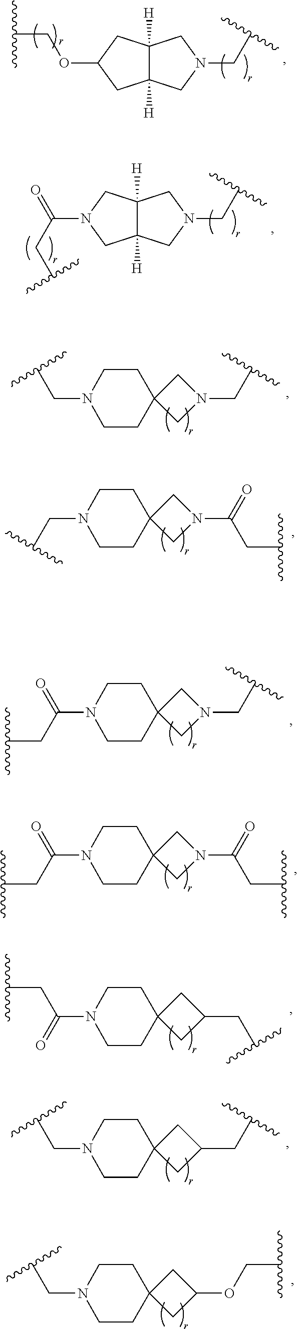 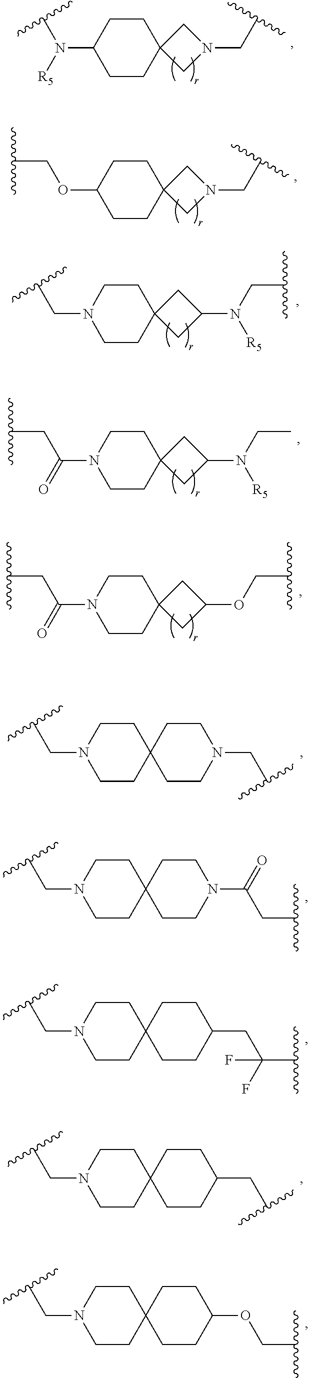 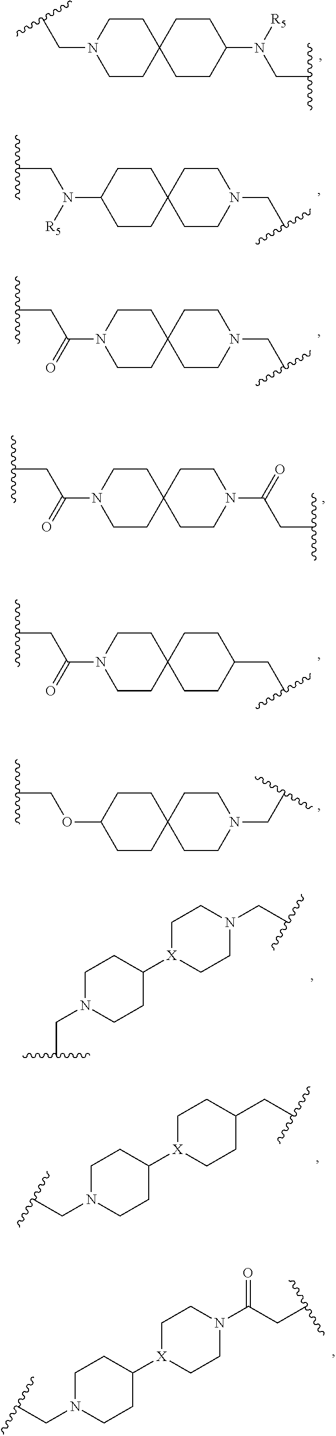 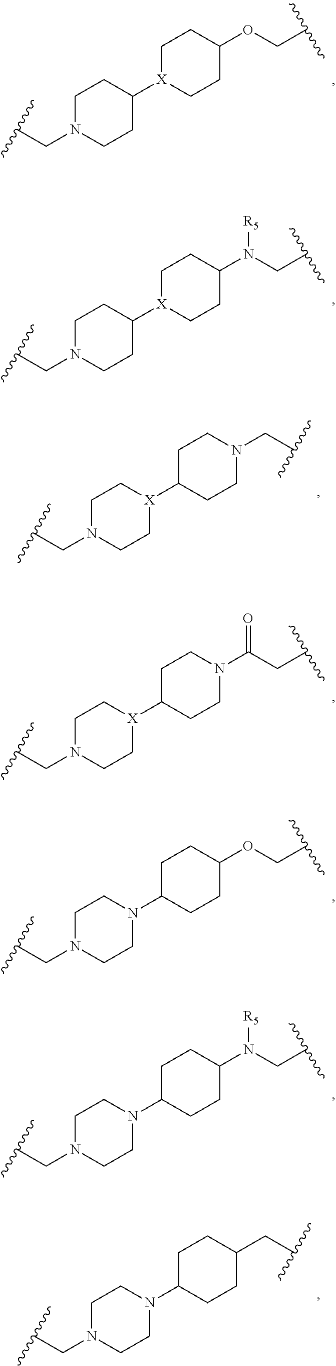 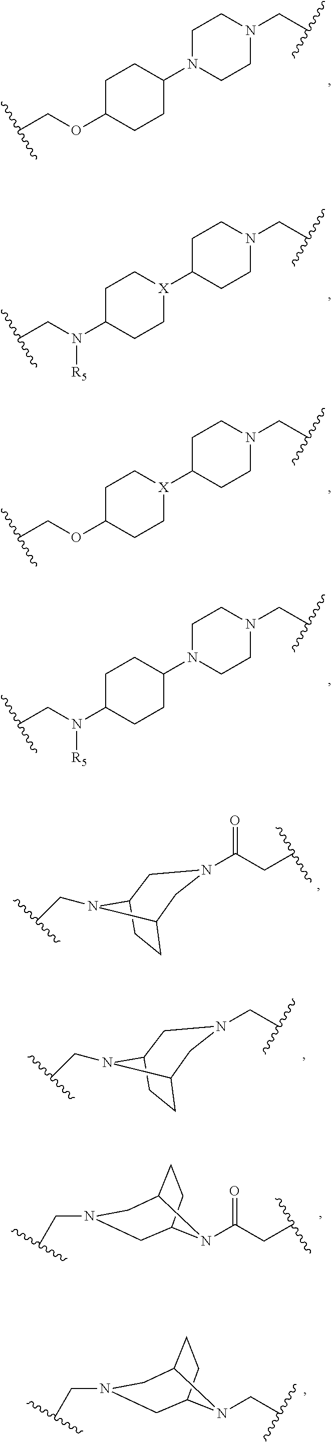 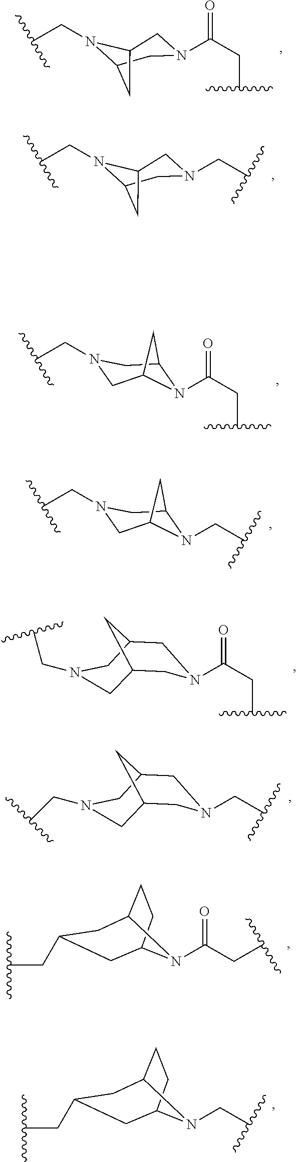 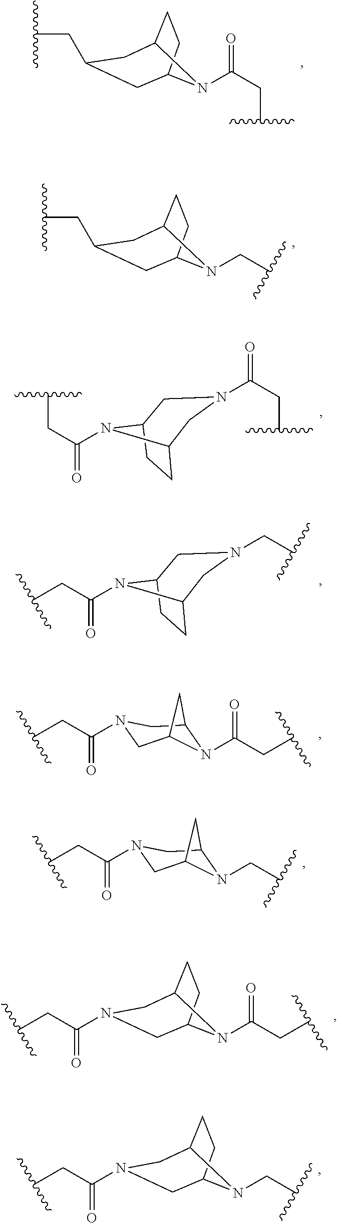 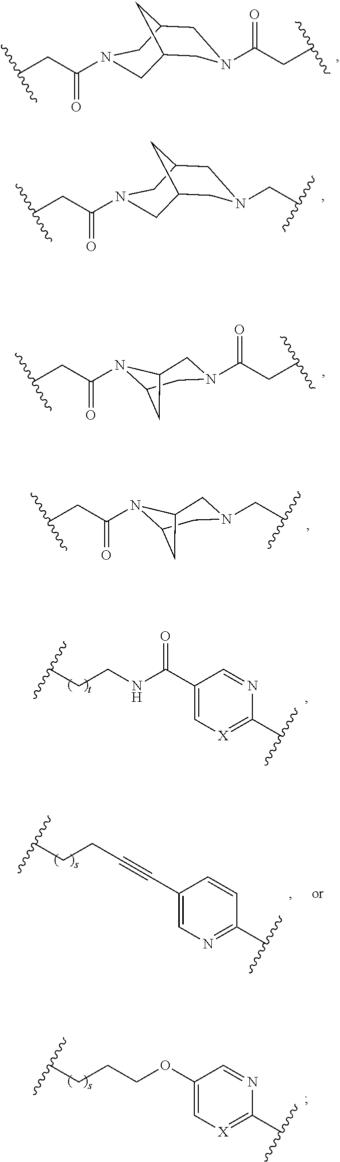
wherein:
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2;
each s is independently 1, 2, 3, or 4;
each t is independently 1, 2, 3, 4, or 5; and
each u is independently 0, 1, 2, 3, 4, or 5.

50. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is a bond.

51. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is substituted or unsubstituted, C₁₋₁₃ alkylene, substituted or unsubstituted, C₂₋₁₃ alkenylene, or substituted or unsubstituted, C₂₋₁₃ alkynylene.

52. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is substituted or unsubstituted, C₁₋₁₃ alkylene, substituted or unsubstituted, C₂₋₁₃ alkenylene, or substituted or unsubstituted, C₂₋₁₃ alkynylene, wherein one or more backbone carbon atoms in the substituted or unsubstituted, C₁₋₁₃ alkylene, substituted or unsubstituted, C₂₋₁₃ alkenylene, or substituted or unsubstituted, C₂₋₁₃ alkynylene are independently replaced with —O—, —NR₅—, —S(═O)—, —S(═O)₂—, or —C(═O)—.

53. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is substituted or unsubstituted, monocyclic heterocyclylene or substituted or unsubstituted, bicyclic heterocyclylene.

54. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is -(substituted or unsubstituted, monocyclic heterocyclylene)-(substituted or unsubstituted, C₁ alkylene)-, -(substituted or unsubstituted, bicyclic heterocyclylene)-(substituted or unsubstituted, C₁ alkylene)-, -(substituted or unsubstituted, C₁ alkylene)-(substituted or unsubstituted, monocyclic heterocyclylene)-, -(substituted or unsubstituted, C₁ alkylene)-(substituted or unsubstituted, bicyclic heterocyclylene)-, —(substituted or unsubstituted, C₂ alkylene)-(substituted or unsubstituted, bicyclic carbocyclylene)-, -(substituted or unsubstituted, bicyclic carbocyclylene)-(substituted or unsubstituted, C₂ alkylene)-, optionally wherein one backbone carbon atom in the substituted or unsubstituted, C₁ alkylene or C₂ alkylene is replaced with —O—, —NR₅—, —S(═O)—, —S(═O)₂—, or —C(═O)—.

55. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is -(substituted or unsubstituted, C₁ alkylene)-(substituted or unsubstituted, monocyclic heterocyclylene)-(substituted or unsubstituted, C₁ alkylene)- or -(substituted or unsubstituted, C₁ alkylene)-(substituted or unsubstituted, bicyclic heterocyclylene)-(substituted or unsubstituted, C₁ alkylene)-, optionally wherein the backbone carbon atom in each instance of the substituted or unsubstituted, C₁ alkylene is independently replaced with —O—, —NR₅—, —S(═O)—, —S(═O)₂—, or —C(═O)—.

56. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is -(substituted or unsubstituted, monocyclic heterocyclylene)₂-, —(substituted or unsubstituted, monocyclic heterocyclylene)₂-(substituted or unsubstituted, C₁ alkylene)-, -(substituted or unsubstituted, C₁ alkylene)-(substituted or unsubstituted, monocyclic heterocyclylene)₂-, -(substituted or unsubstituted, C₁ alkylene)-(substituted or unsubstituted, monocyclic heterocyclylene)₂-(substituted or unsubstituted, C₁ alkylene)-, -(substituted or unsubstituted, monocyclic heterocyclylene)-(substituted or unsubstituted, monocyclic carbocyclylene)-, or -(substituted or unsubstituted, monocyclic carbocyclylene)-(substituted or unsubstituted, monocyclic heterocyclylene)-, optionally wherein the backbone carbon atom in each instance of the substituted or unsubstituted, C₁ alkylene is independently replaced with —O—, —NR₅—, —S(═O)—, —S(═O)₂—, or —C(═O)—.

57. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is -(substituted or unsubstituted, C₁ alkylene)-(substituted or unsubstituted, monocyclic heterocyclylene)- or -(substituted or unsubstituted, monocyclic heterocyclylene)-(substituted or unsubstituted, C₁ alkylene)-.

58. The compound of any one of claims 1-49, or a pharmaceutically acceptable salt, solvate, hydrate, polymorph, co-crystal, tautomer, stereoisomer, isotopically labeled compound, or prodrug thereof, wherein L₃ is 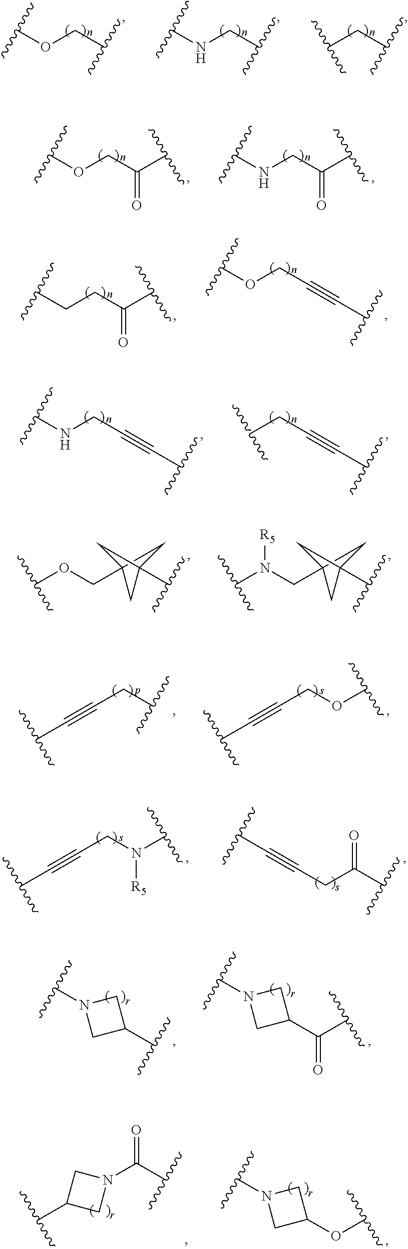 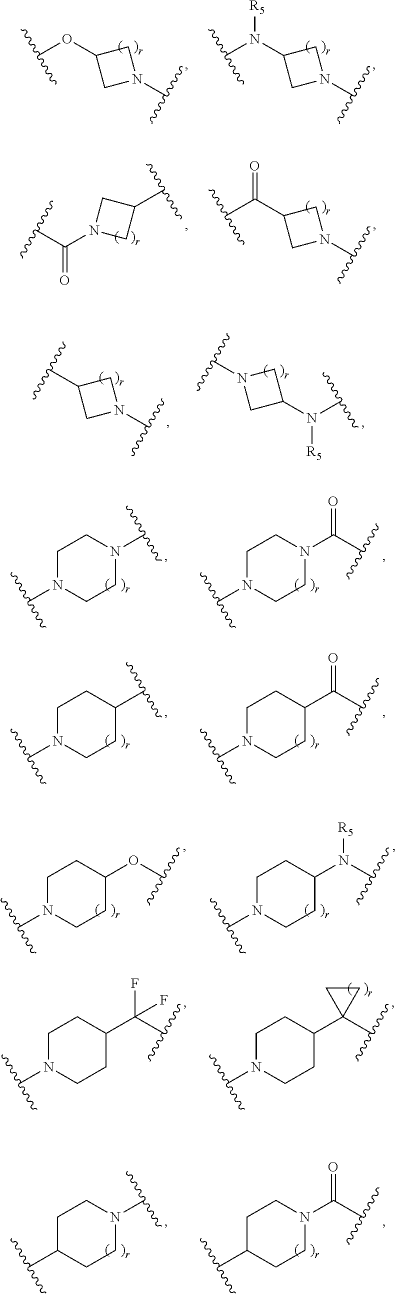 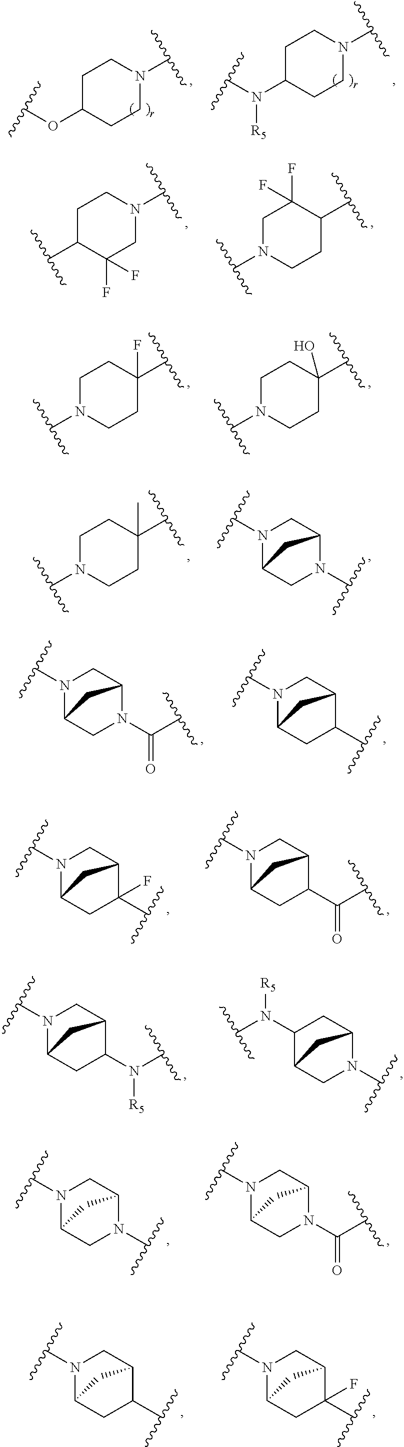 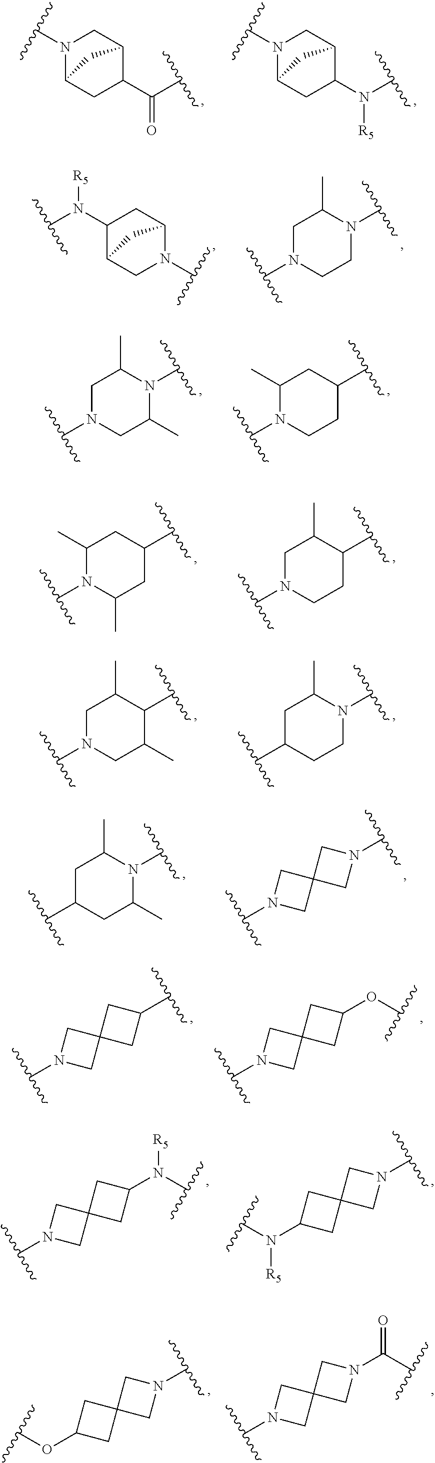 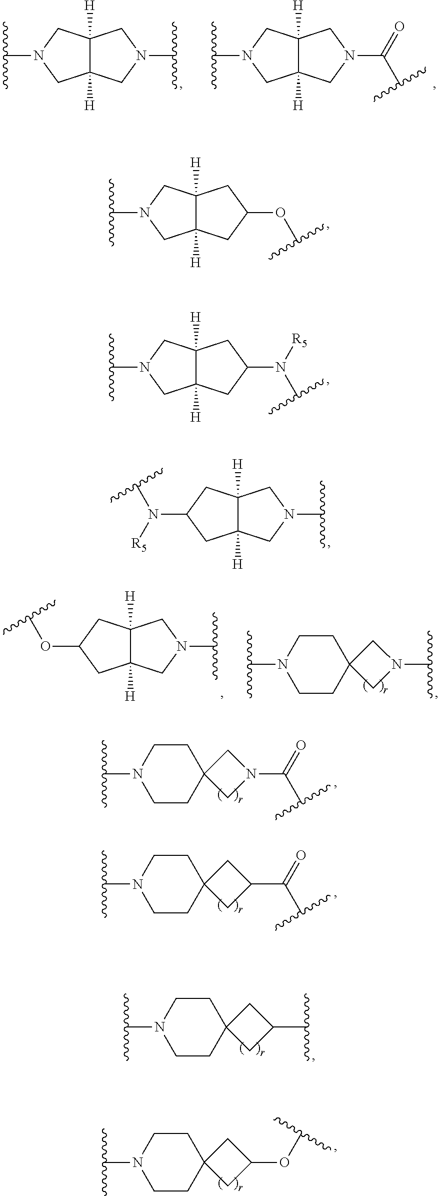 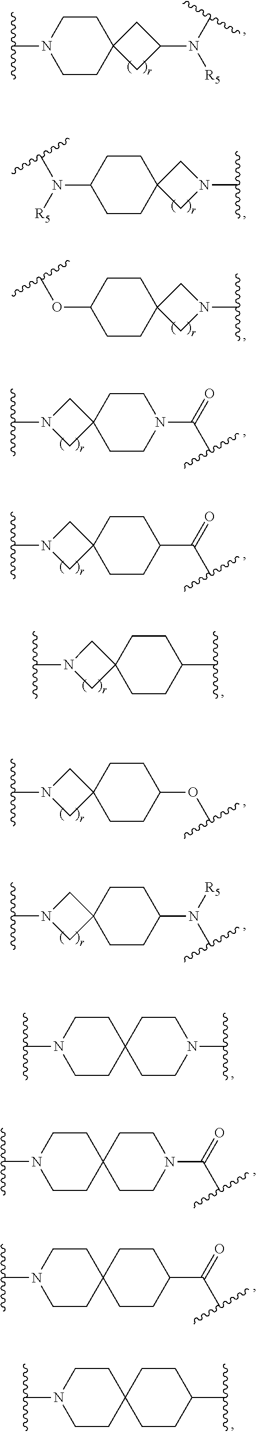 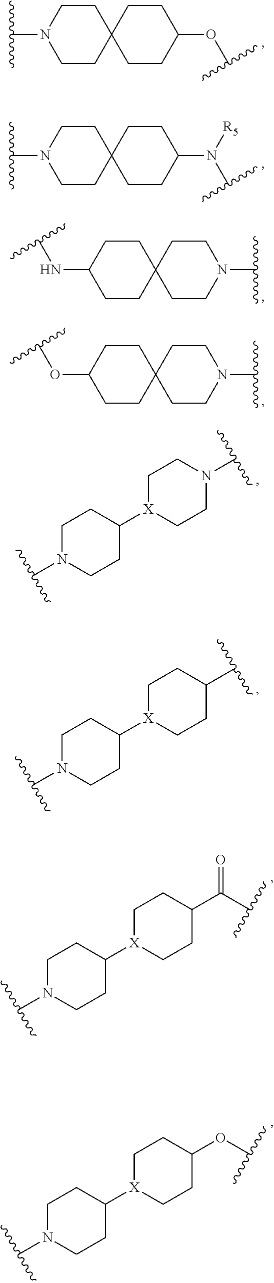 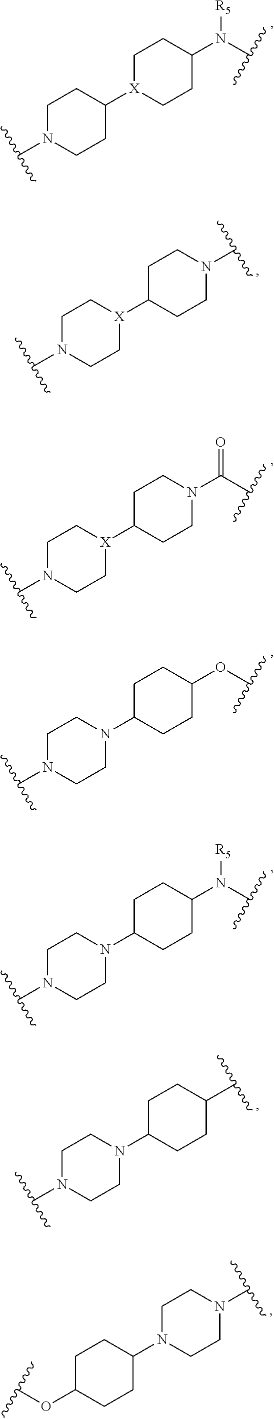 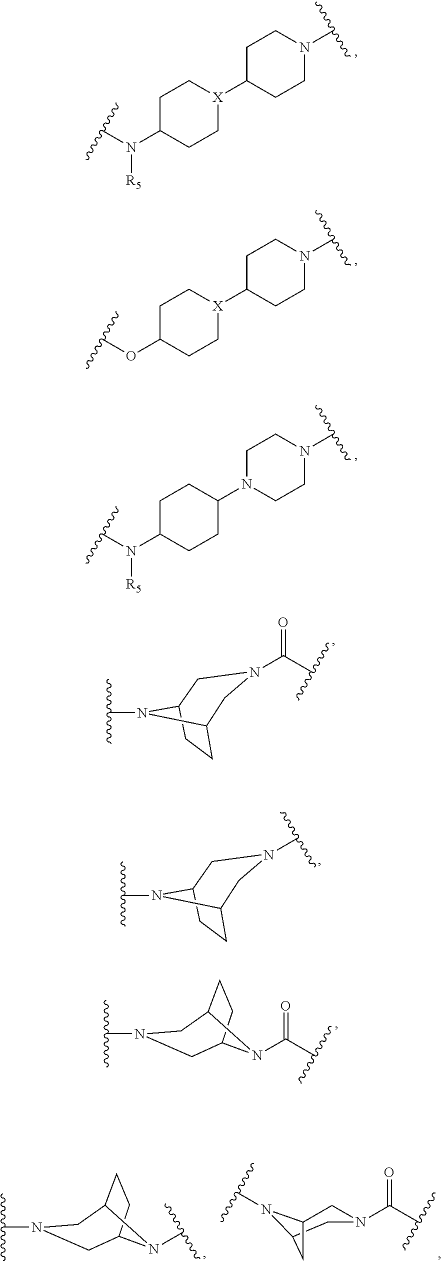 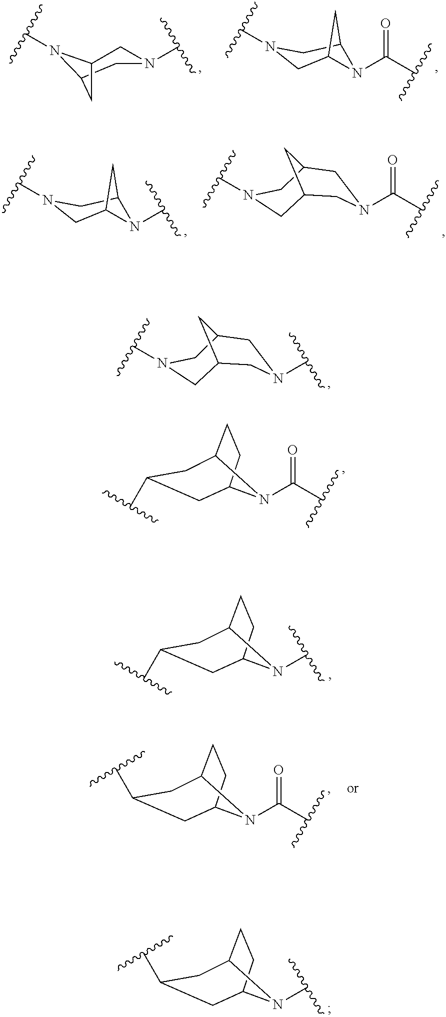
wherein:
each n is independently 0, 1, 2, 3, 4, 5, 6, 7, 8, 9, or 10;
each p is independently 0, 1, 2, 3, or 4;
each q is independently 1, 2, or 3;
each r is independently 1 or 2;
each s is independently 1, 2, 3, or 4;
each t is independently 1, 2, 3, 4, or 5; and
each u is independently 0, 1, 2, 3, 4, or 5.

59. The compound of any one of claims 1-58, or a pharmaceutically acceptable salt, solvate, hydrate, polymorph, co-crystal, tautomer, stereoisomer, isotopically labeled compound, or prodrug thereof, wherein at least one instance of R₅ is hydrogen.

60. The compound of any one of claims 1-58, or a pharmaceutically acceptable salt, solvate, hydrate, polymorph, co-crystal, tautomer, stereoisomer, isotopically labeled compound, or prodrug thereof, wherein at least one instance of R₅ is substituted or unsubstituted alkyl or substituted or unsubstituted carbocyclyl.

61. The compound of any one of claims 1-60, or a pharmaceutically acceptable salt, solvate, hydrate, polymorph, co-crystal, tautomer, stereoisomer, isotopically labeled compound, or prodrug thereof, wherein L₃ is attached to the 2 or 3 position.

62. The compound of any one of claims 1-60, or a pharmaceutically acceptable salt, solvate, hydrate, polymorph, co-crystal, tautomer, stereoisomer, isotopically labeled compound, or prodrug thereof, wherein L₃ is attached to the 1 or 4 position.

63. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

| Compound | |
|---|---|
| Number | Formula |
| 1 | 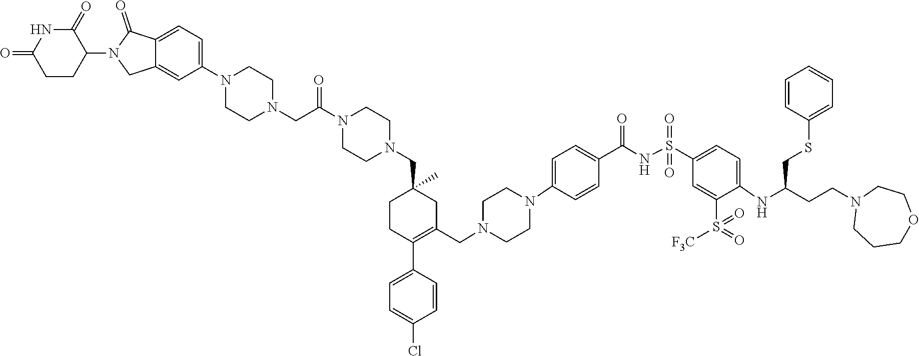 [SMILES: C[C@@]1(CN2CCN(C(=O)CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 2 | 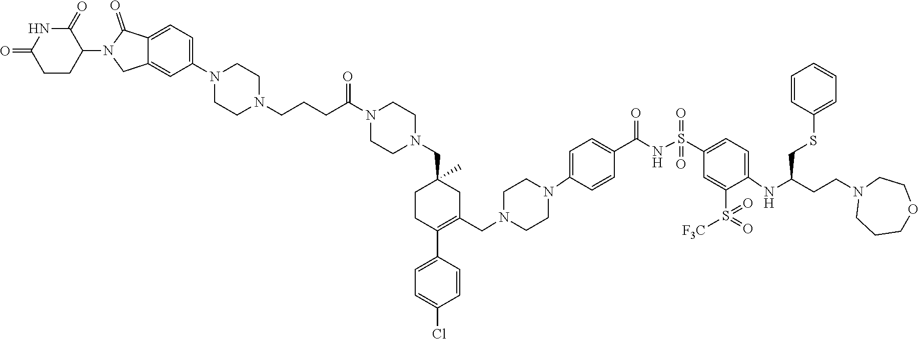 [SMILES: C[C@@]1(CN2CCN(C(=O)CCCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 3 | 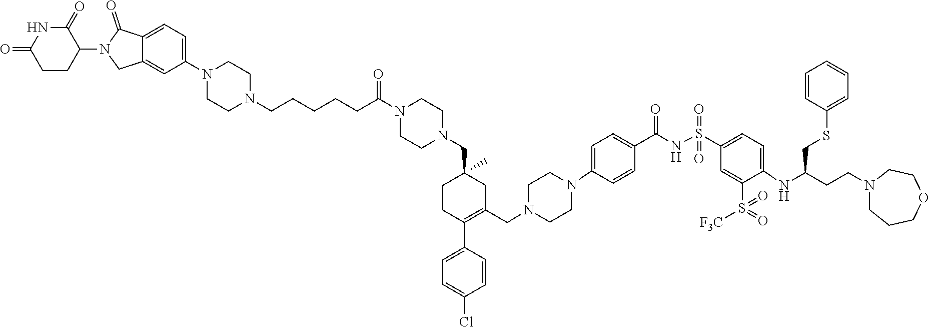 [SMILES: C[C@@]1(CN2CCN(C(=O)CCCCCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 4 | 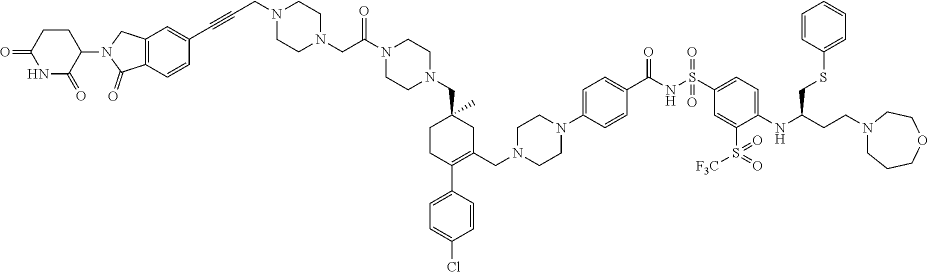 [SMILES: C[C@@]1(CN2CCN(C(=O)CN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 5 | 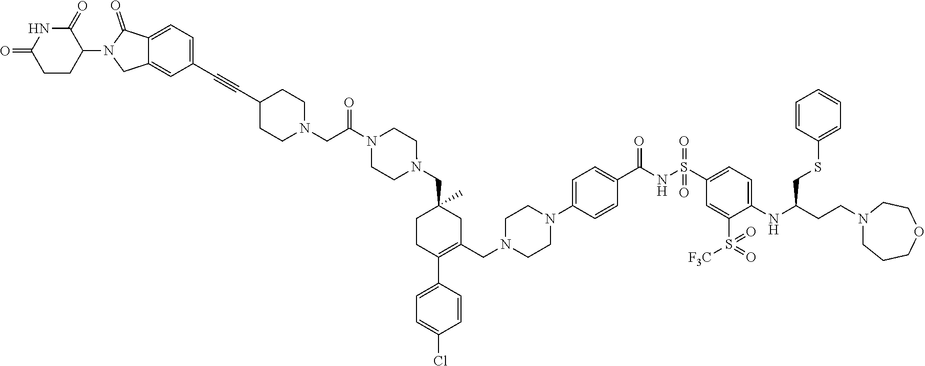 [SMILES: C[C@@]1(CN2CCN(C(=O)CN3CCC(C#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 6 | 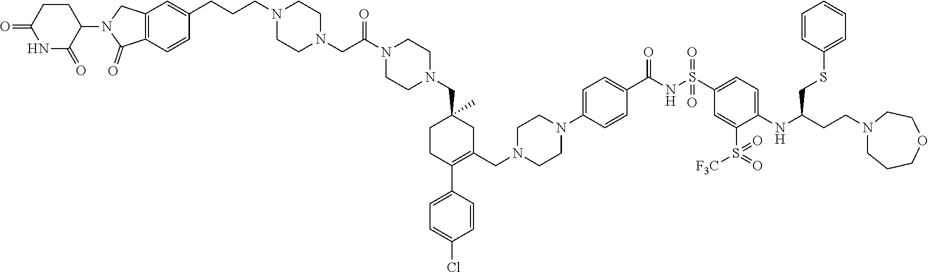 [SMILES: C[C@@]1(CN2CCN(C(=O)CN3CCN(CCCc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 7 | 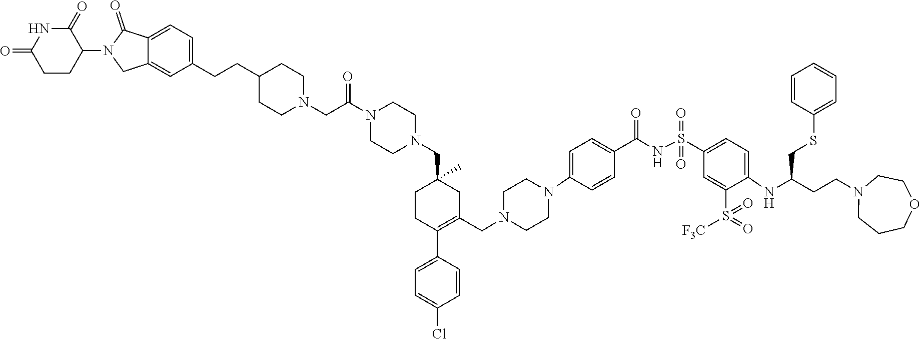 [SMILES: C[C@@]1(CN2CCN(C(=O)CN3CCC(CCc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 8 | 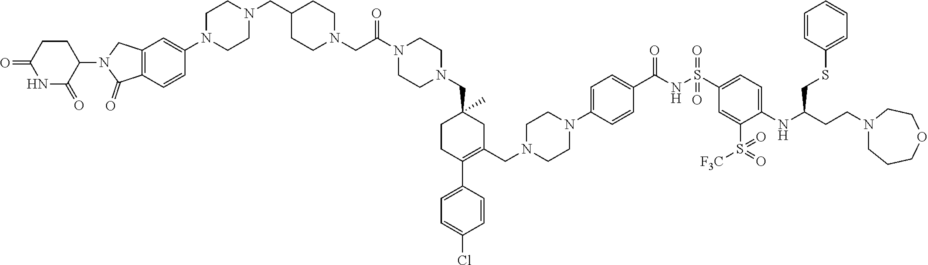 [SMILES: C[C@@]1(CN2CCN(C(=O)CN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 9 | 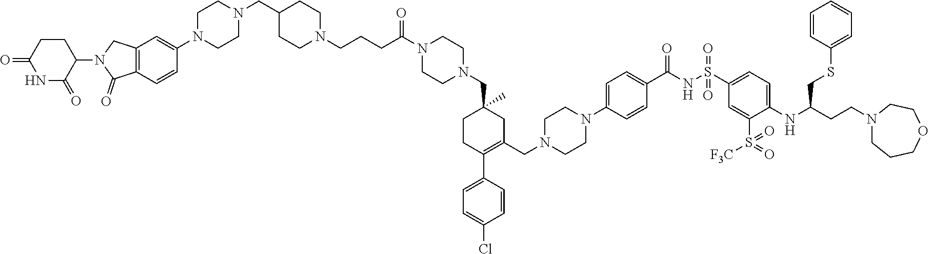 [SMILES: C[C@@]1(CN2CCN(C(=O)CCCN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 10 | 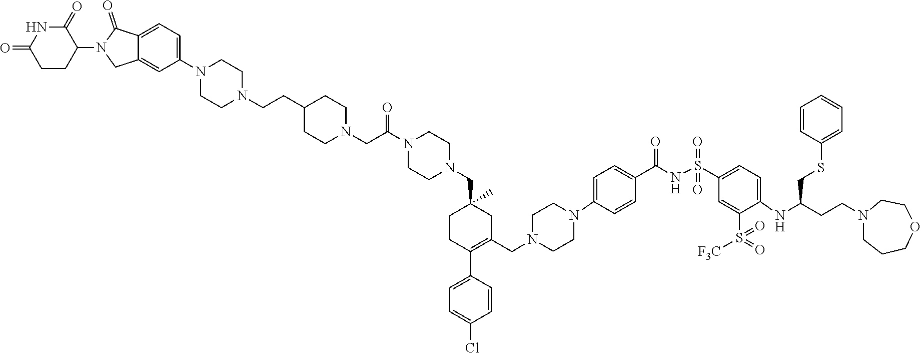 [SMILES: C[C@@]1(CN2CCN(C(=O)CN3CCC(CCN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 11 | 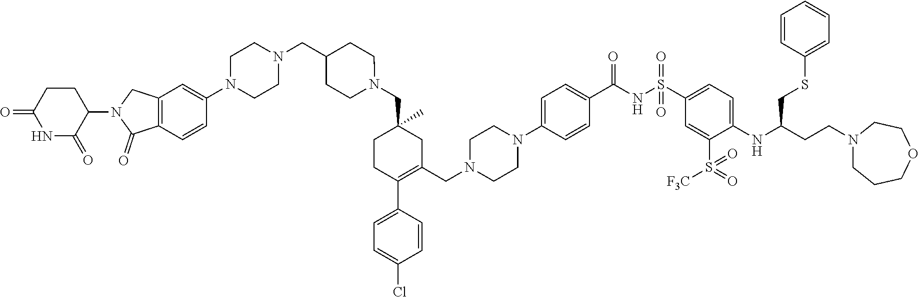 [SMILES: C[C@@]1(CN2CCC(CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 12 | 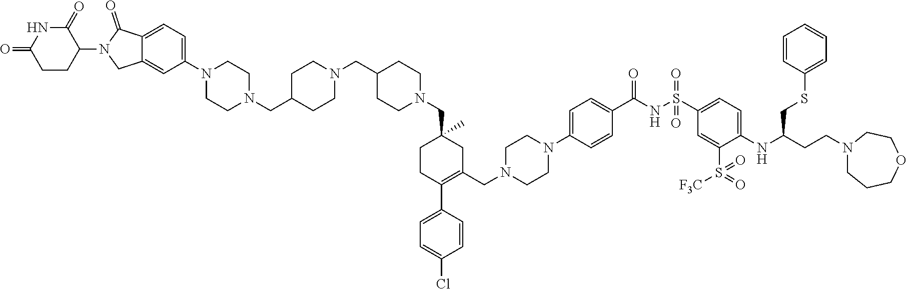 [SMILES: C[C@@]1(CN2CCC(CN3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 13 | 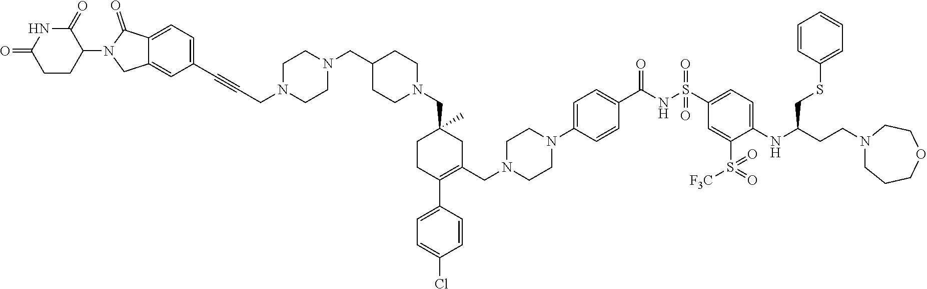 [SMILES: C[C@@]1(CN2CCC(CN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 14 | 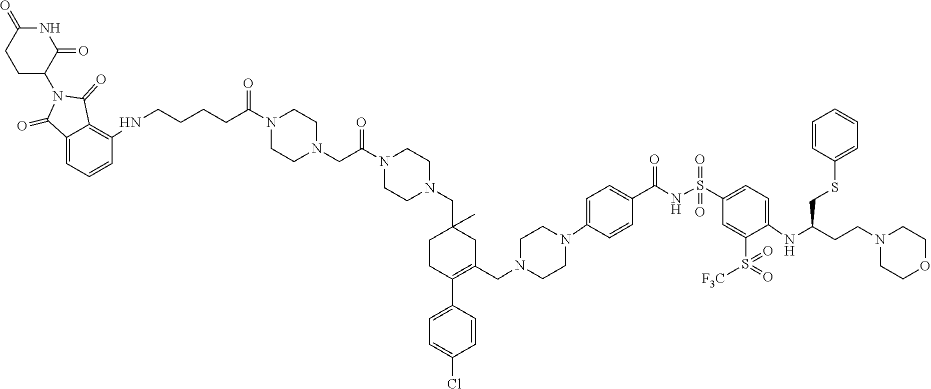 [SMILES: CC1(CN2CCN(C(=O)CN3CCN(C(=O)CCCCNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 15 | 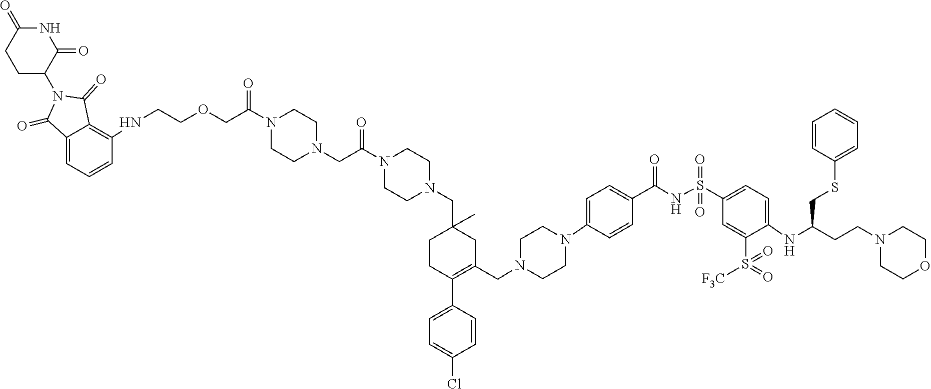 [SMILES: CC1(CN2CCN(C(=O)CN3CCN(C(=O)COCCNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 16 | 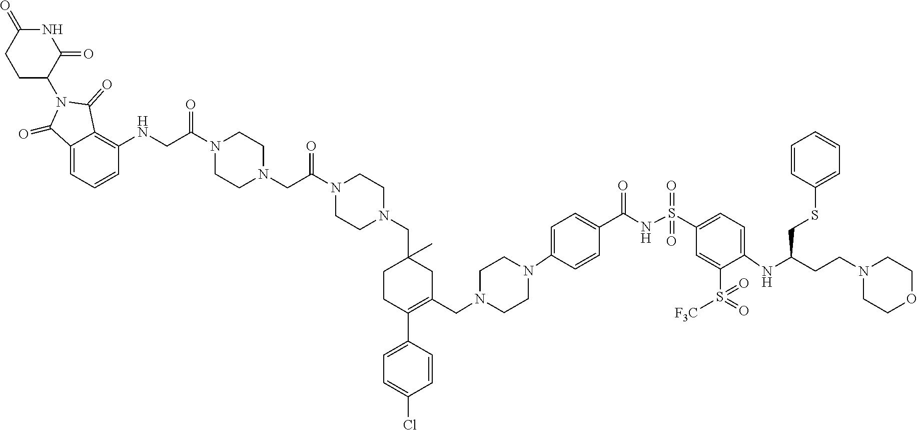 [SMILES: CC1(CN2CCN(C(=O)CN3CCN(C(=O)CNc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 17 | 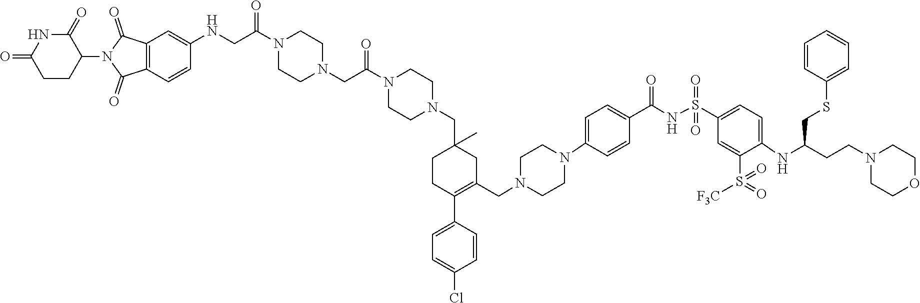 [SMILES: CC1(CN2CCN(C(=O)CN3CCN(C(=O)CNc4ccc5c(c4)C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 18 | 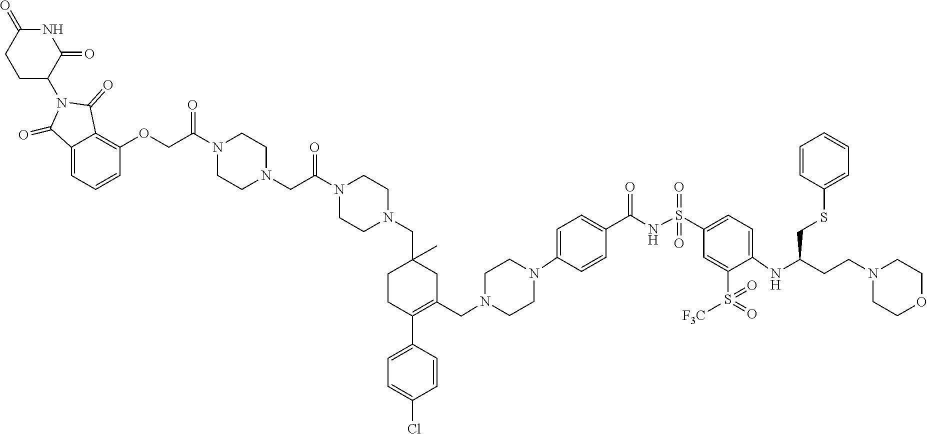 [SMILES: CC1(CN2CCN(C(=O)CN3CCN(C(=O)COc4cccc5c4C(=O)N(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 19 | 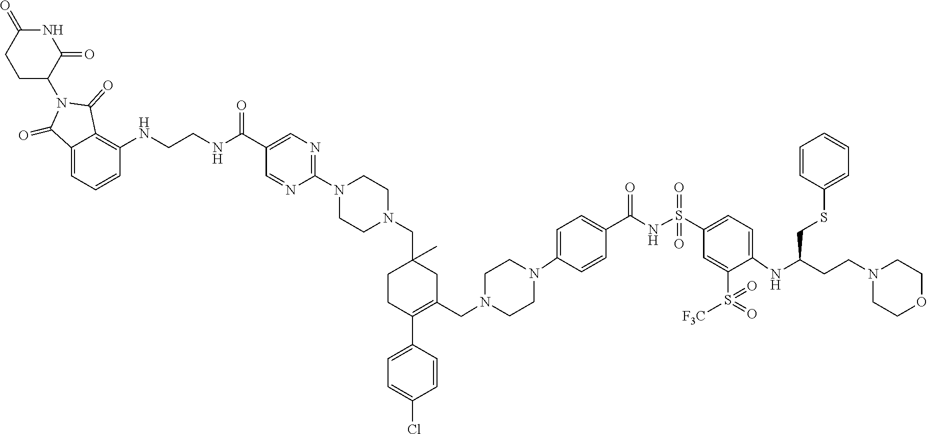 [SMILES: CC1(CN2CCN(c3ncc(C(=O)NCCNc4cccc5c4C(=O)N(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 20 | 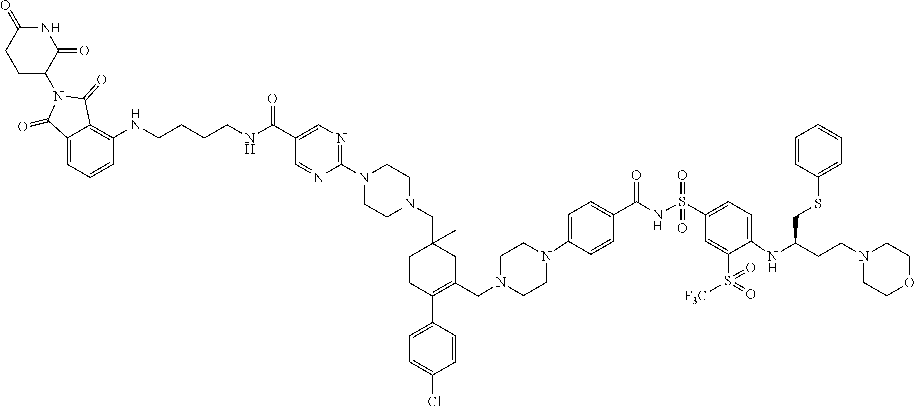 [SMILES: CC1(CN2CCN(c3ncc(C(=O)NCCCCNc4cccc5c4C(=O)N(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 21 | 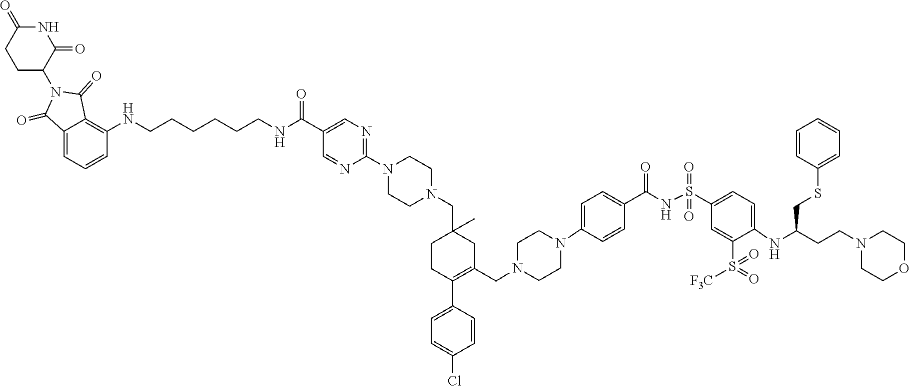 [SMILES: CC1(CN2CCN(c3ncc(C(=O)NCCCCCCNc4cccc5c4C(=O)N(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 22 | 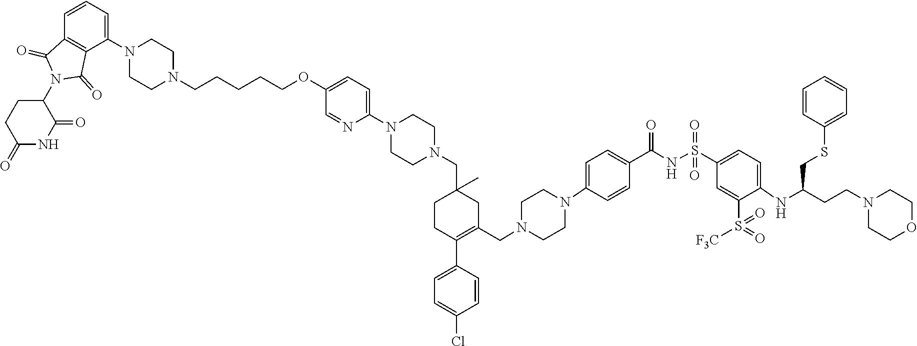 [SMILES: CC1(CN2CCN(c3ccc(OCCCCCN4CCN(c5cccc6c5C(=O)N(C5CCC(=O)NC5=O)C6=O)CC4)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 23 | 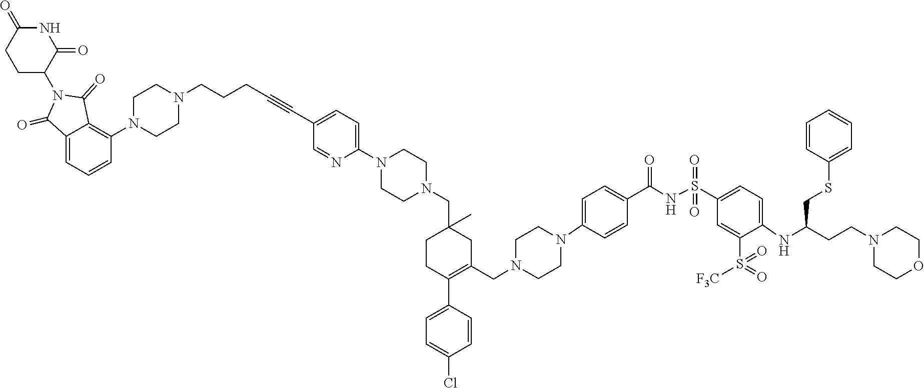 [SMILES: CC1(CN2CCN(c3ccc(C#CCCCN4CCN(c5cccc6c5C(=O)N(C5CCC(=O)NC5=O)C6=O)CC4)cn3)CC2)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 24 | 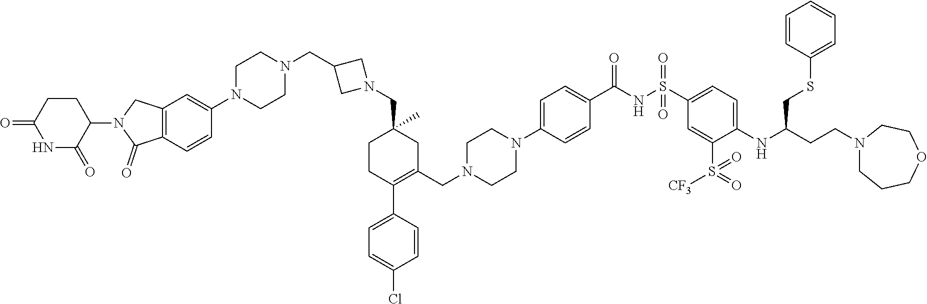 [SMILES: C[C@@]1(CN2CC(CN3CCN(c4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 25 | 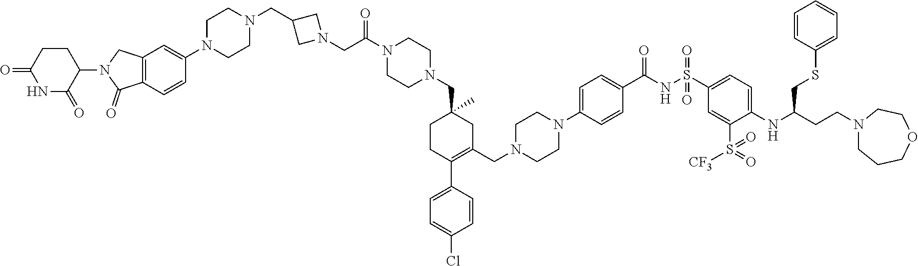 [SMILES: C[C@@]1(CN2CCN(C(=O)CN3CC(CN4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 26 | 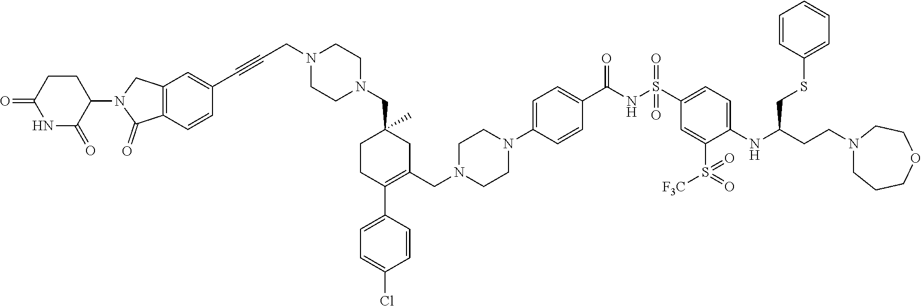 [SMILES: C[C@@]1(CN2CCN(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 27 | 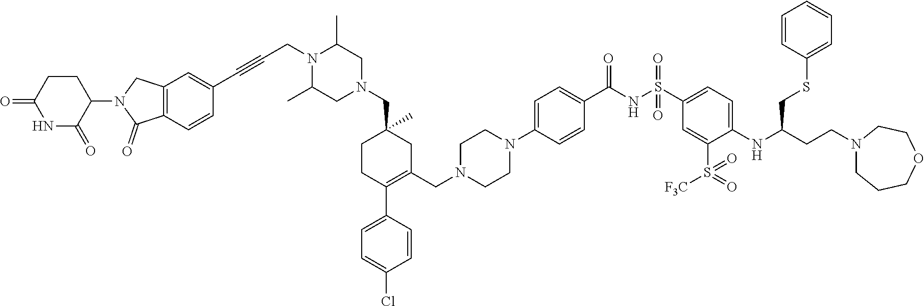 [SMILES: CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC(C)N1CC#Cc1ccc2c(c1)CN(C1CCC(=O)NC1=O)C2=O] |
| 28 | 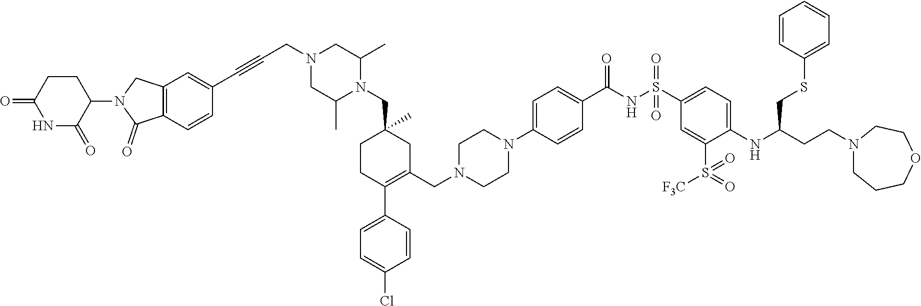 [SMILES: CC1CN(CC#Cc2ccc3c(c2)CN(C2CCC(=O)NC2=O)C3=O)CC(C)N1C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 29 | 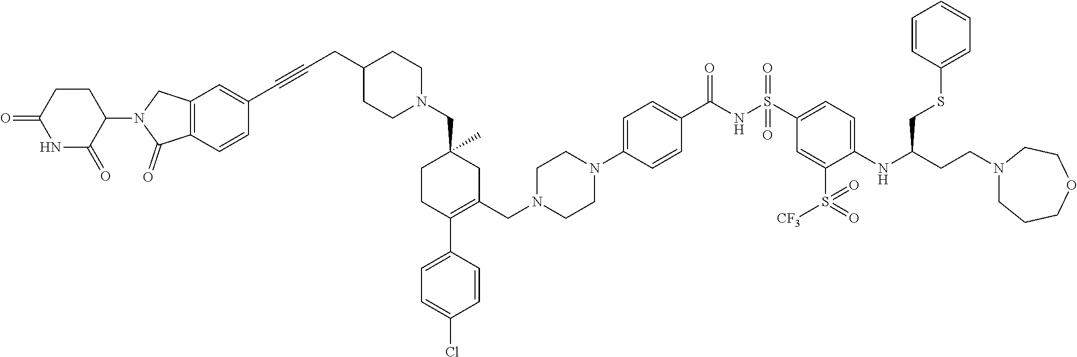 [SMILES: C[C@@]1(CN2CCC(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 30 | 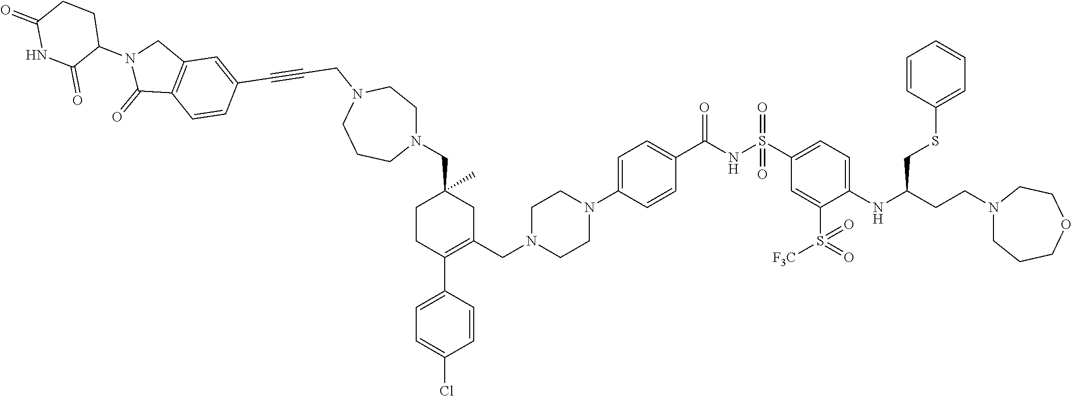 [SMILES: C[C@@]1(CN2CCCN(CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 31 | 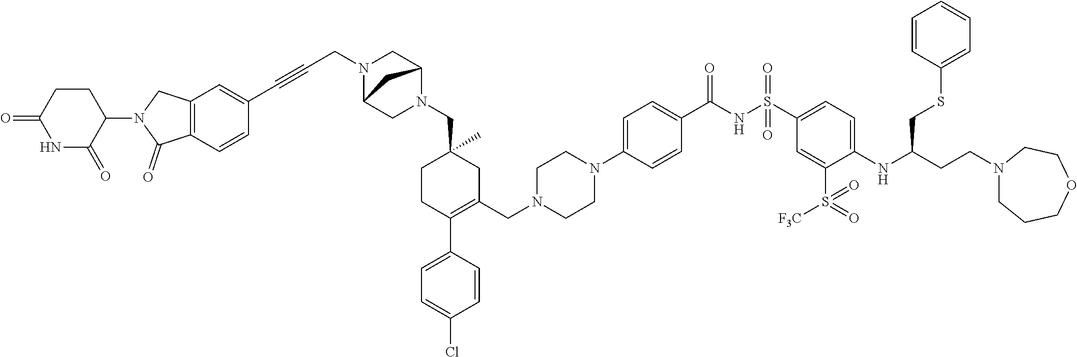 [SMILES: C[C@@]1(CN2C[C@H]3C[C@@H]2CN3CC#Cc2ccc3c(c2)CN(C2CCC(=O)NC2=O)C3=O)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 32 | 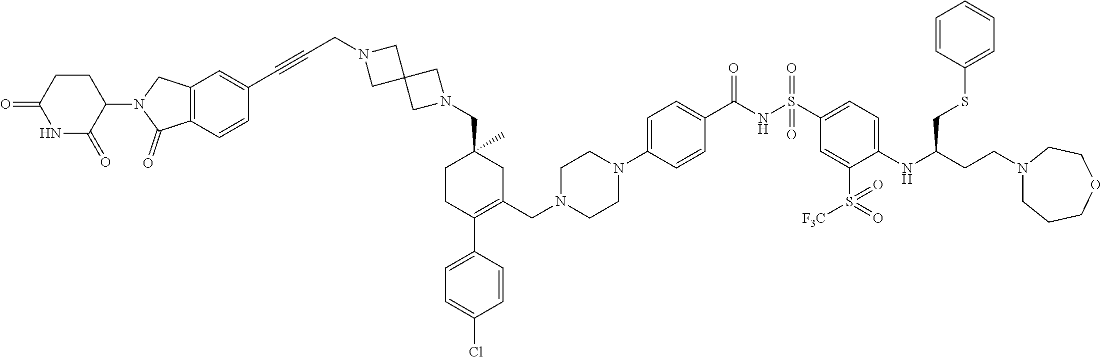 [SMILES: C[C@@]1(CN2CC3(CN(CC#Cc4ccc5c(c4)CN(C4CCC(=O)NC4=O)C5=O)C3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 33 | 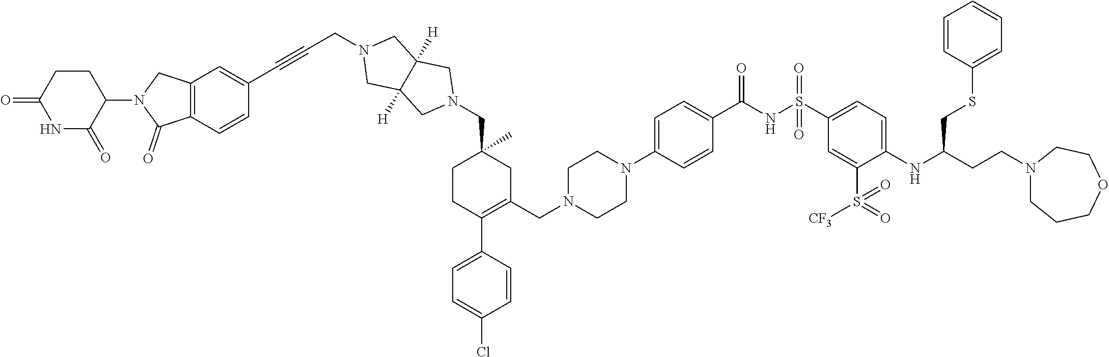 [SMILES: C[C@@]1(CN2C[C@H]3CN(CC#Cc4ccc5c(c4)CN(C4CCC(=O)NC4=O)C5=O)C[C@H]3C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 34 | 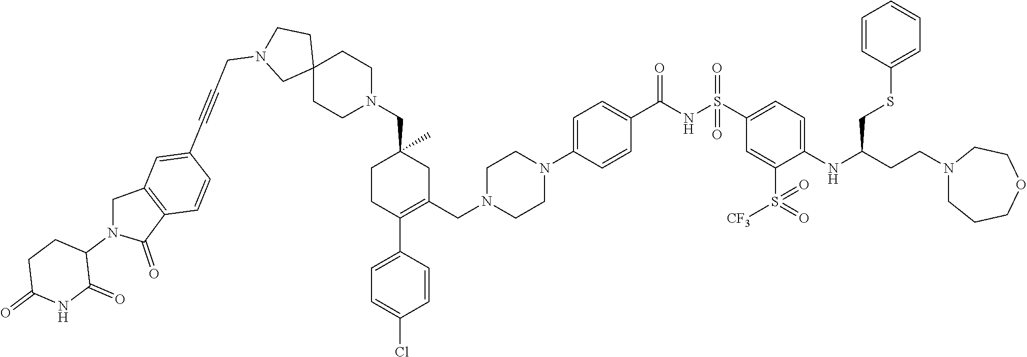 [SMILES: C[C@@]1(CN2CCC3(CCN(CC#Cc4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 35 | 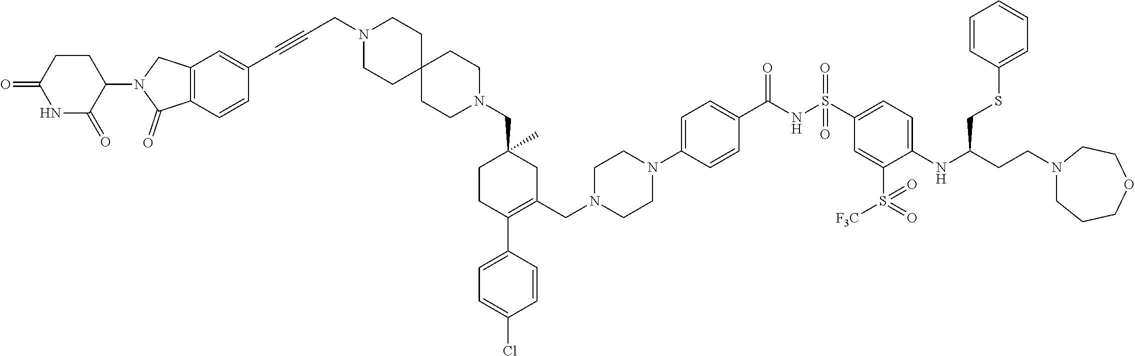 [SMILES: C[C@@]1(CN2CCC3(CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 36 | 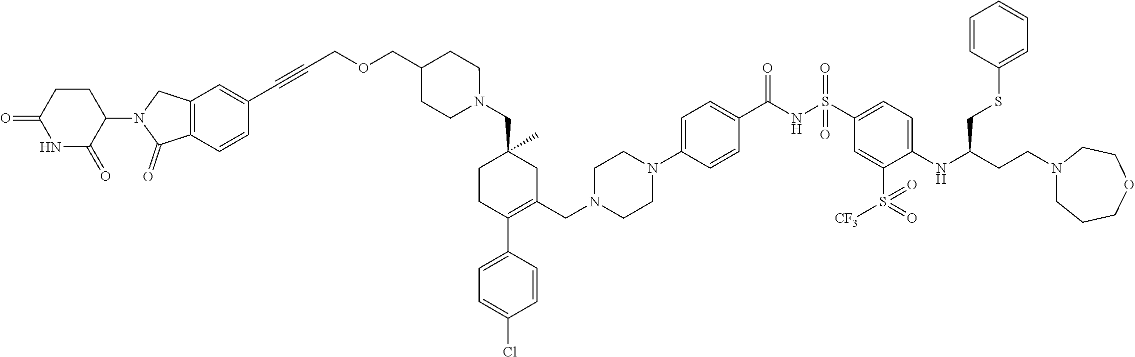 [SMILES: C[C@@]1(CN2CCC(COCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 37 | 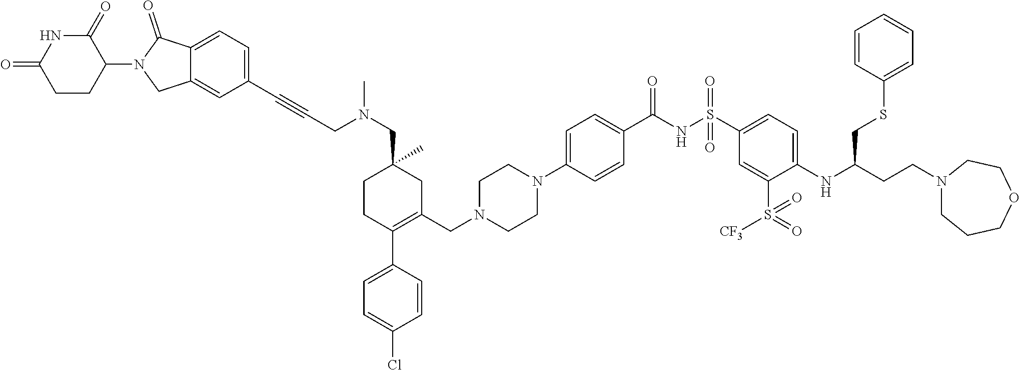 [SMILES: CN(CC#Cc1ccc2c(c1)CN(C1CCC(=O)NC1=O)C2=O)C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 38 | 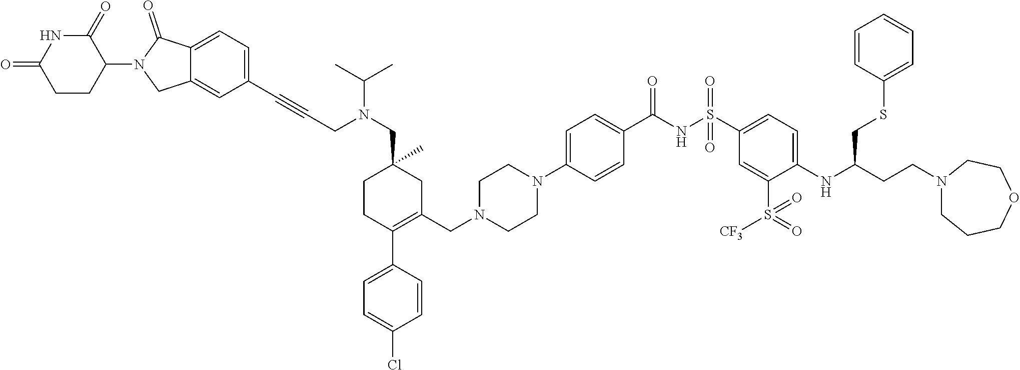 [SMILES: CC(C)N(CC#Cc1ccc2c(c1)CN(C1CCC(=O)NC1=O)C2=O)C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 39 | 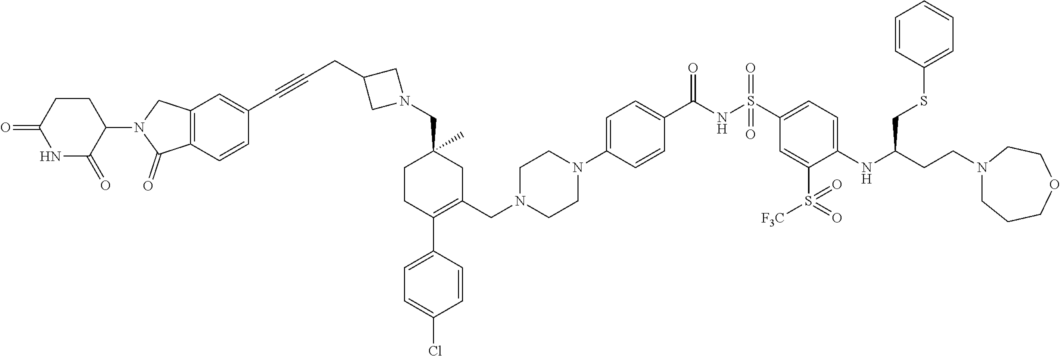 [SMILES: C[C@@]1(CN2CC(CC#Cc3ccc4c(c3)CN(C3CCC(=O)NC3=O)C4=O)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 40 | 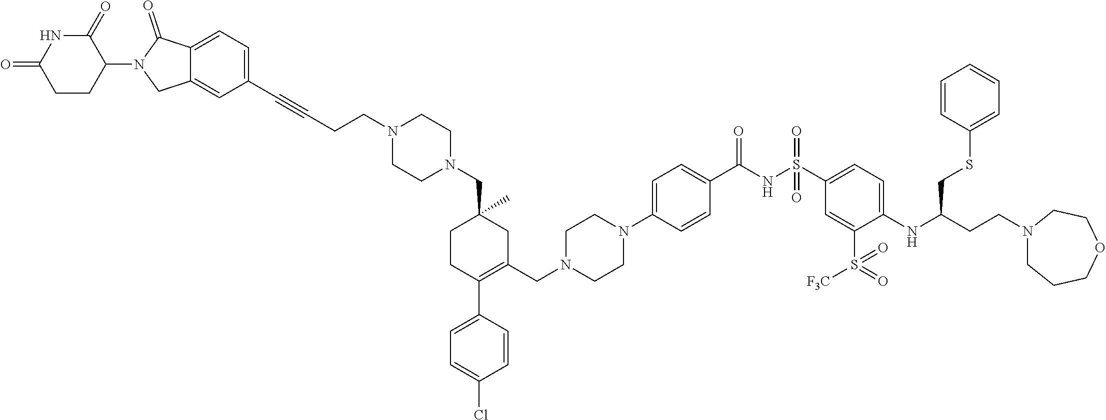 [SMILES: C[C@@]1(CN2CCN(CCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 41 | 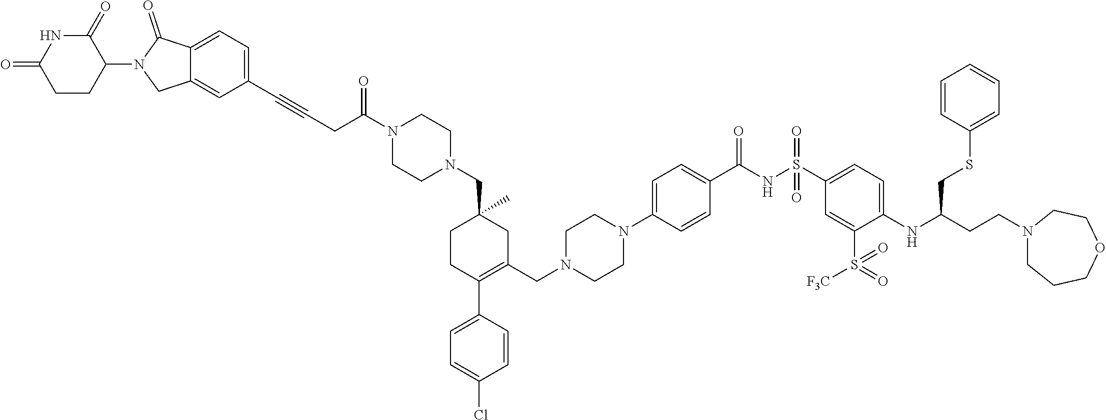 [SMILES: C[C@@]1(CN2CCN(C(=O)CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 42 | 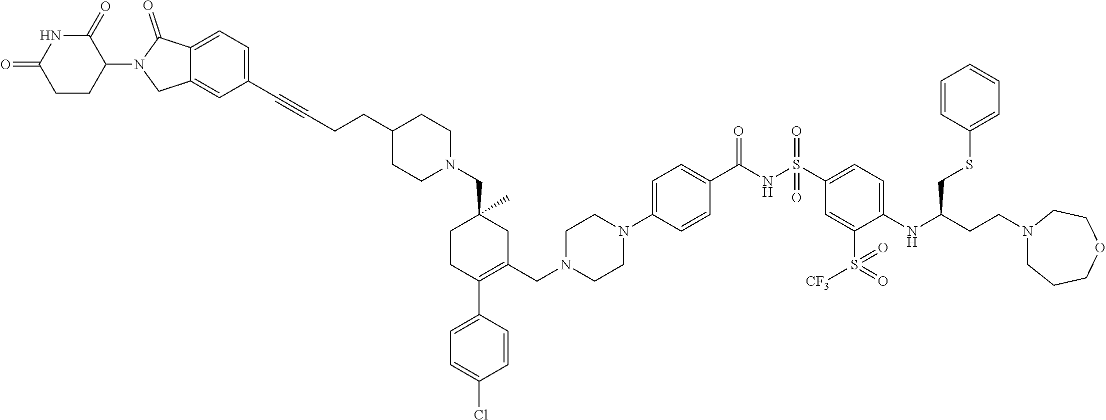 [SMILES: C[C@@]1(CN2CCC(CCC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 43 | 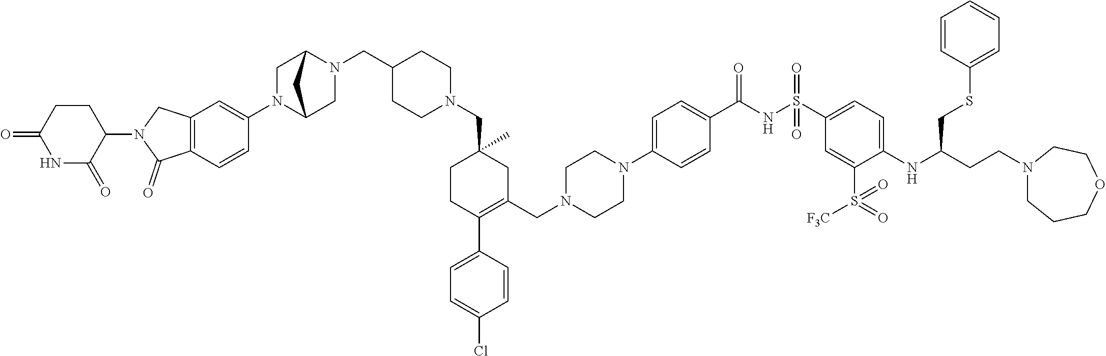 [SMILES: C[C@@]1(CN2CCC(CN3C[C@H]4C[C@@H]3CN4c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 44 | 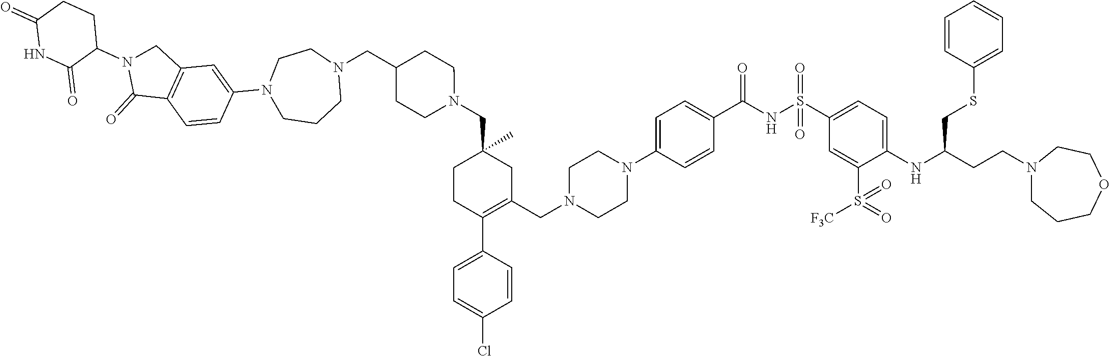 [SMILES: C[C@@]1(CN2CCC(CN3CCCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 45 | 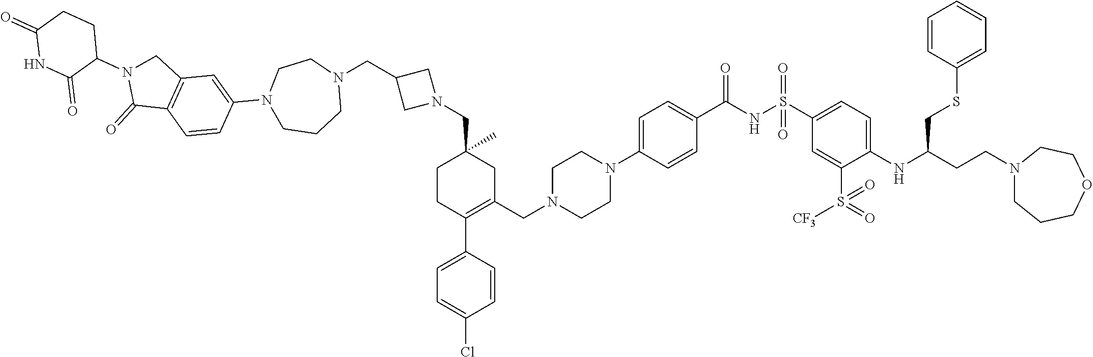 [SMILES: C[C@@]1(CN2CC(CN3CCCN(c4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 46 | 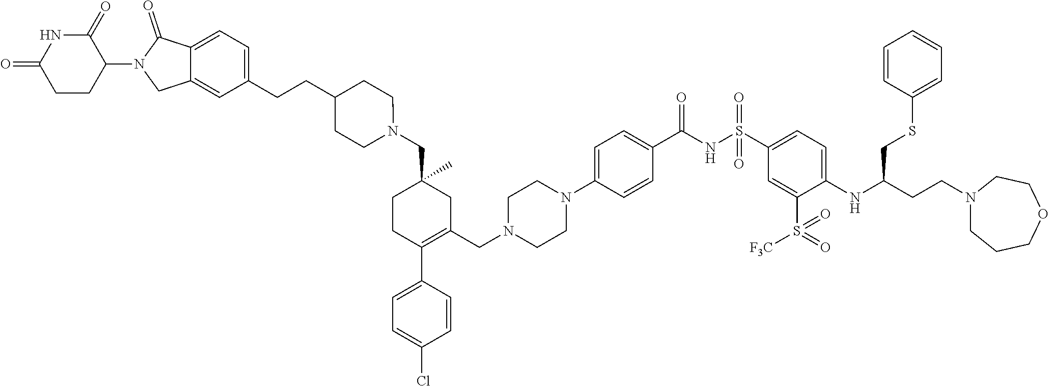 [SMILES: C[C@@]1(CN2CCC(CCc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 47 | 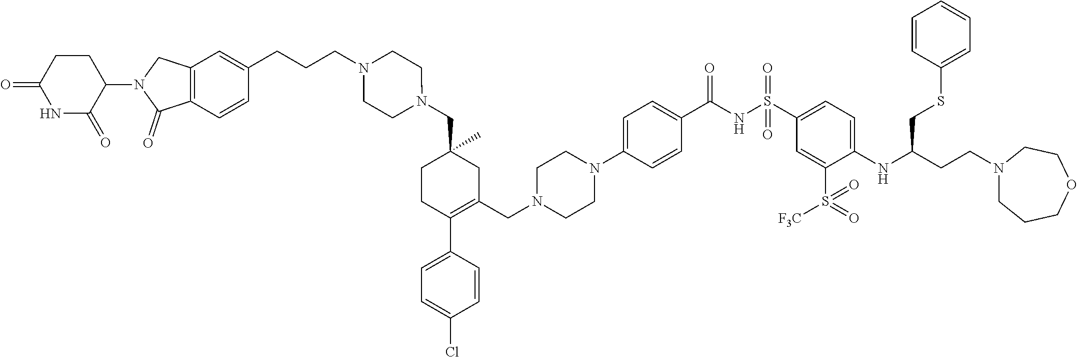 [SMILES: C[C@@]1(CN2CCN(CCCc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 48 | 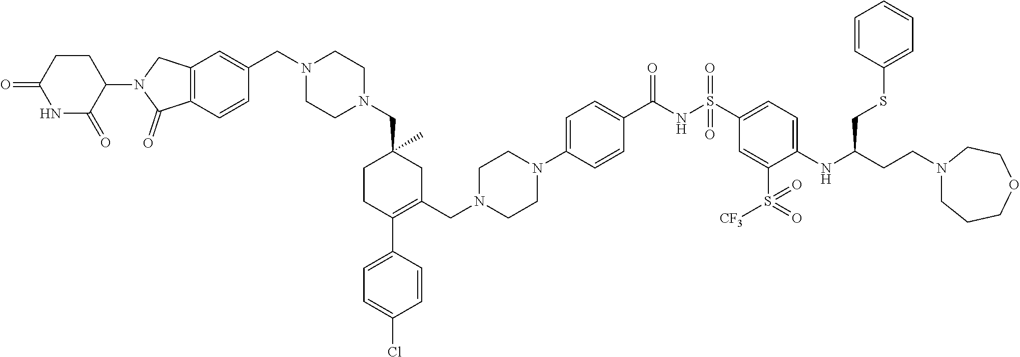 [SMILES: C[C@@]1(CN2CCN(Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 49 | 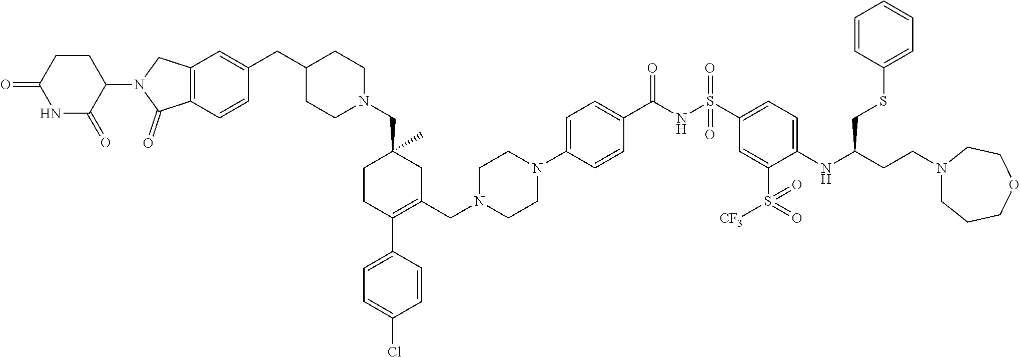 [SMILES: C[C@@]1(CN2CCC(Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 50 | 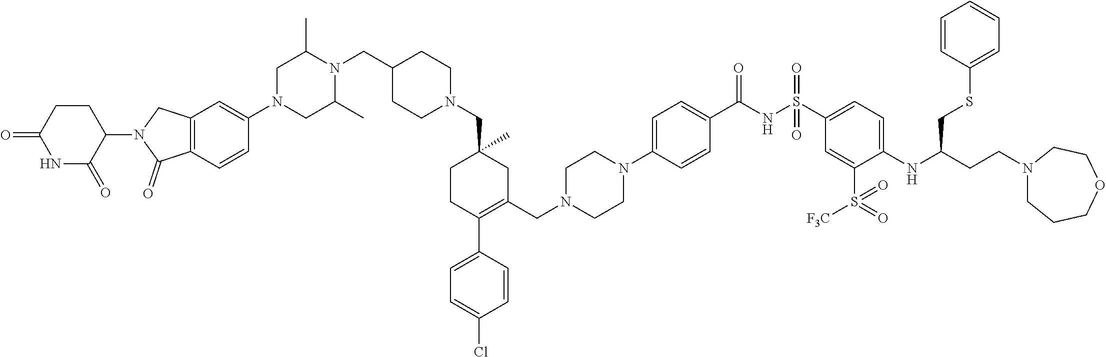 [SMILES: CC1CN(c2ccc3c(c2)CN(C2CCC(=O)NC2=O)C3=O)CC(C)N1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 51 | 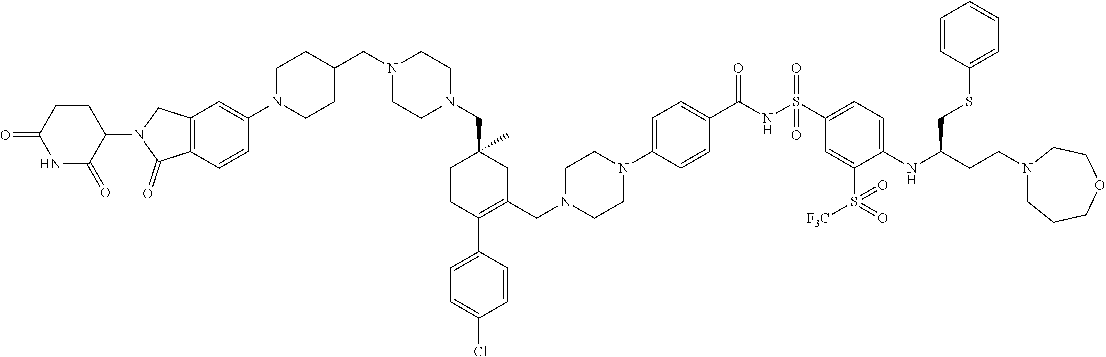 [SMILES: C[C@@]1(CN2CCN(CC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 52 | 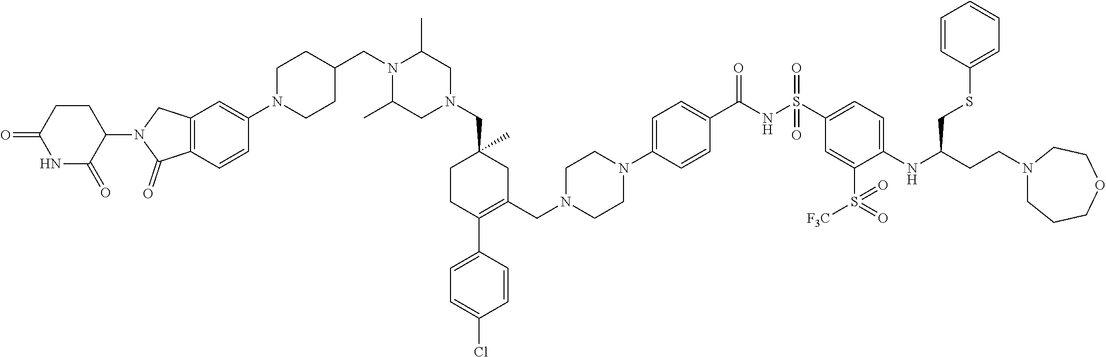 [SMILES: CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC(C)N1CC1CCN(c2ccc3c(c2)CN(C2CCC(=O)NC2=O)C3=O)CC1] |
| 53 | 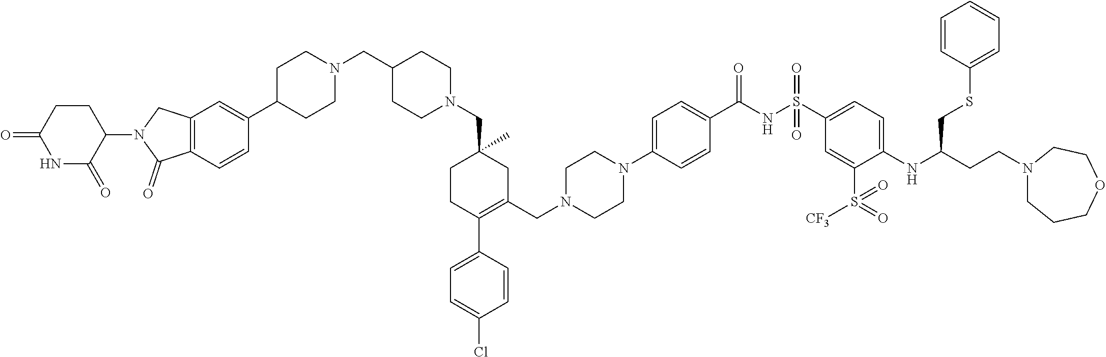 [SMILES: C[C@@]1(CN2CCC(CN3CCC(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 54 | 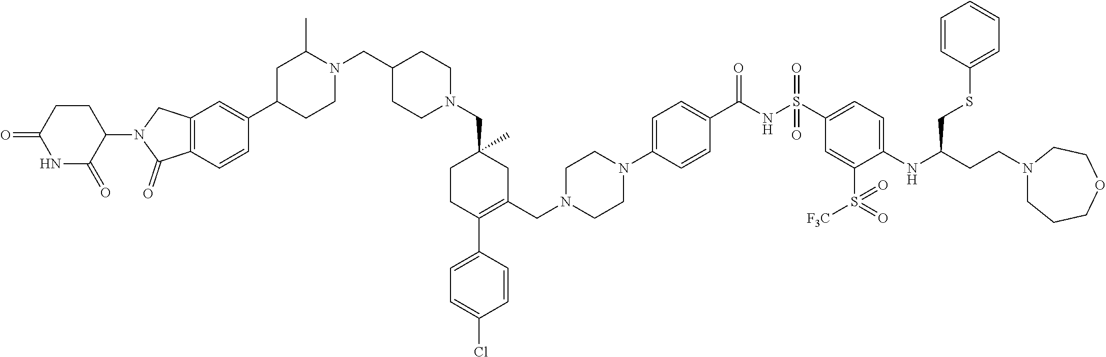 [SMILES: CC1CC(c2ccc3c(c2)CN(C2CCC(=O)NC2=O)C3=O)CCN1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 55 | 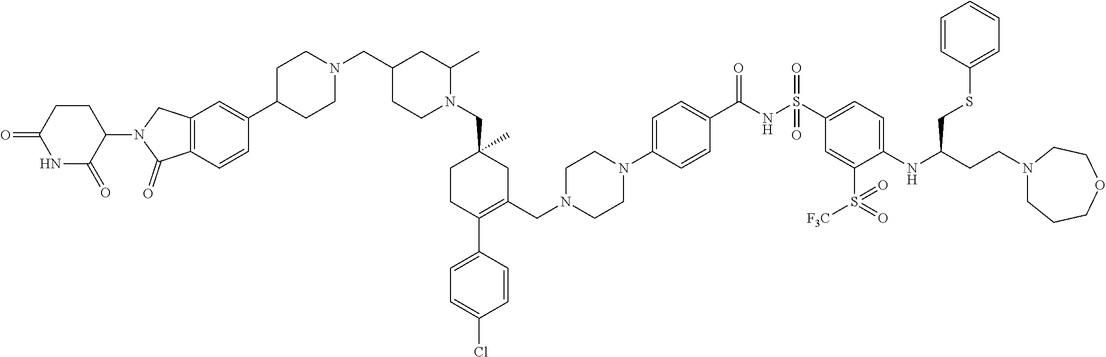 [SMILES: CC1CC(CN2CCC(c3ccc4c(c3)CN(C3CCC(=O)NC3=O)C4=O)CC2)CCN1C[C@]1(C)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 56 | 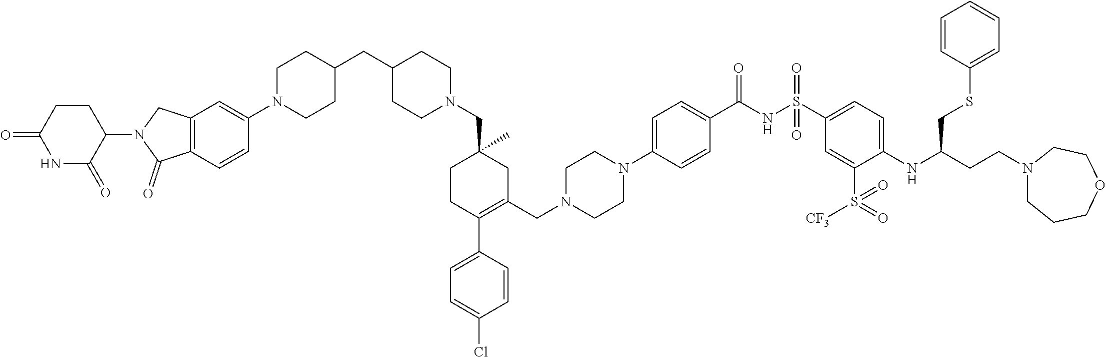 [SMILES: C[C@@]1(CN2CCC(CC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 57 | 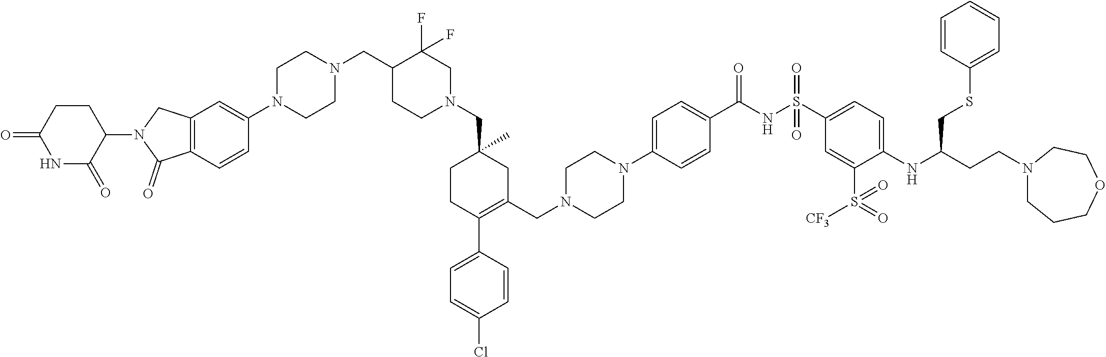 [SMILES: C[C@@]1(CN2CCC(CN3CCN(c4ccc5c(c4)CN(C4CCC(=O)NC4=O)C5=O)CC3)C(F)(F)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 58 | 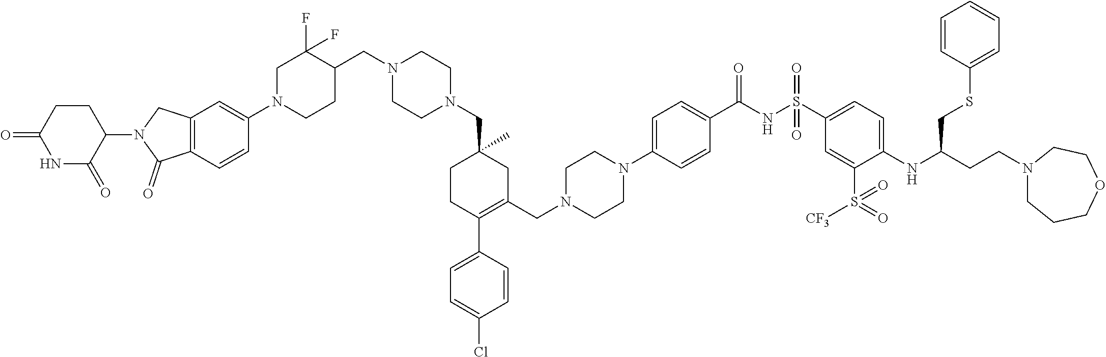 [SMILES: C[C@@]1(CN2CCN(CC3CCN(c4ccc5c(c4)CN(C4CCC(=O)NC4=O)C5=O)CC3(F)F)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 59 | 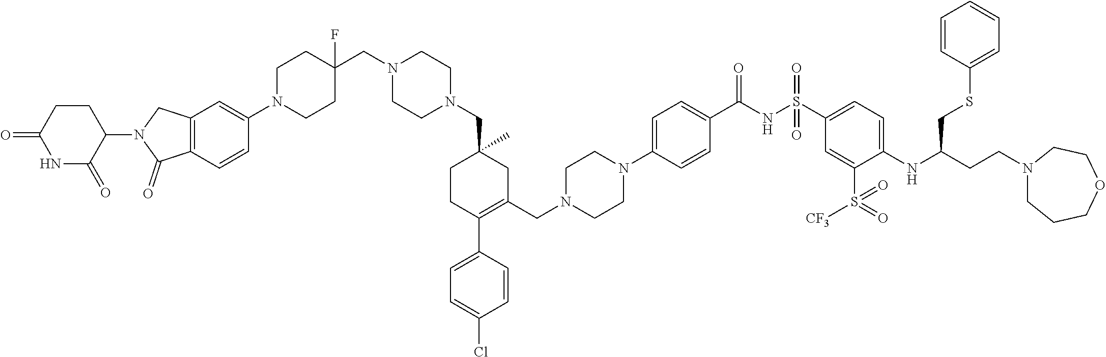 [SMILES: C[C@@]1(CN2CCN(CC3(F)CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 60 | 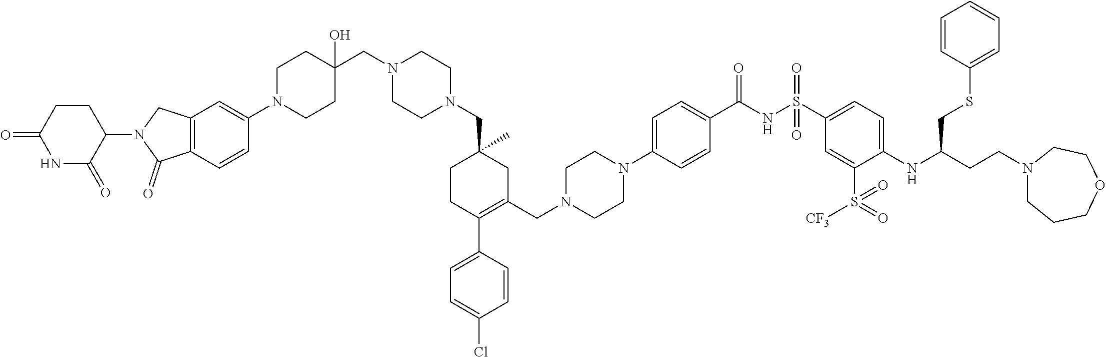 [SMILES: C[C@@]1(CN2CCN(CC3(O)CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 61 | 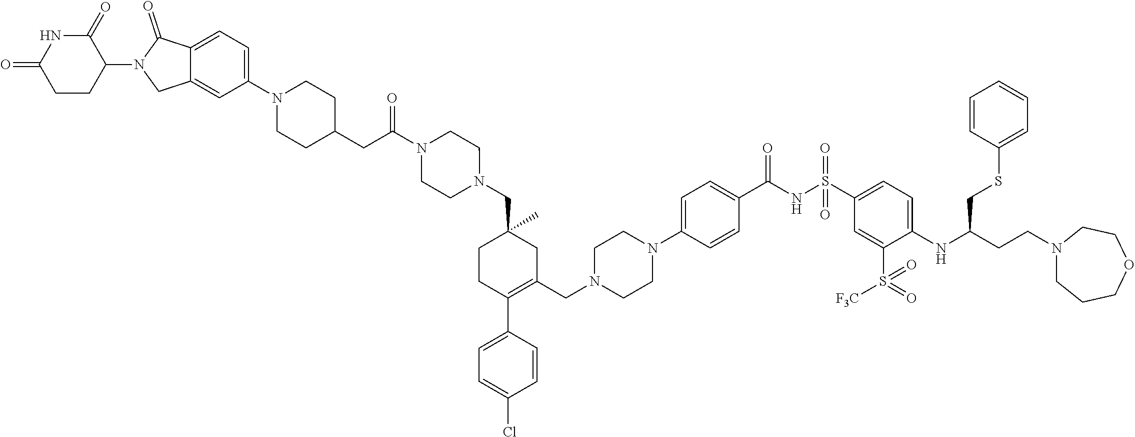 [SMILES: C[C@@]1(CN2CCN(C(=O)CC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 62 | 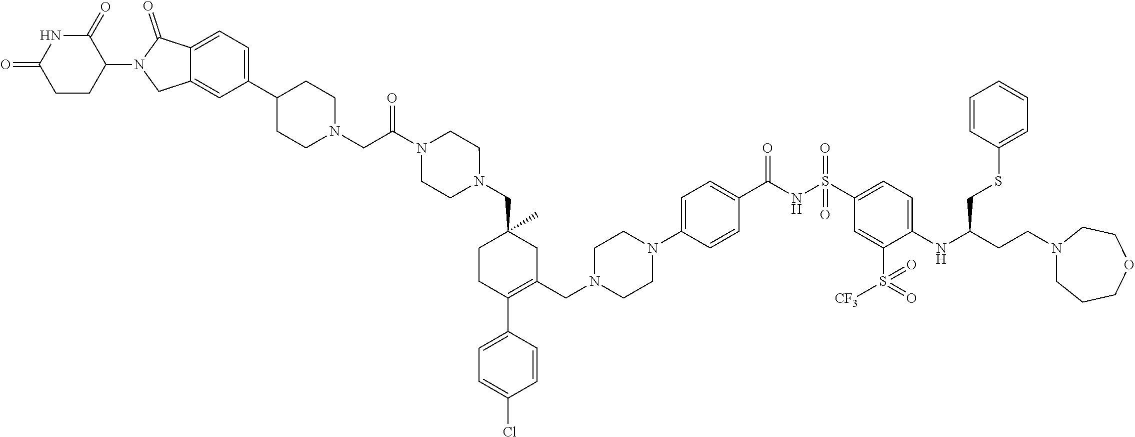 [SMILES: C[C@@]1(CN2CCN(C(=O)CN3CCC(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 63 | 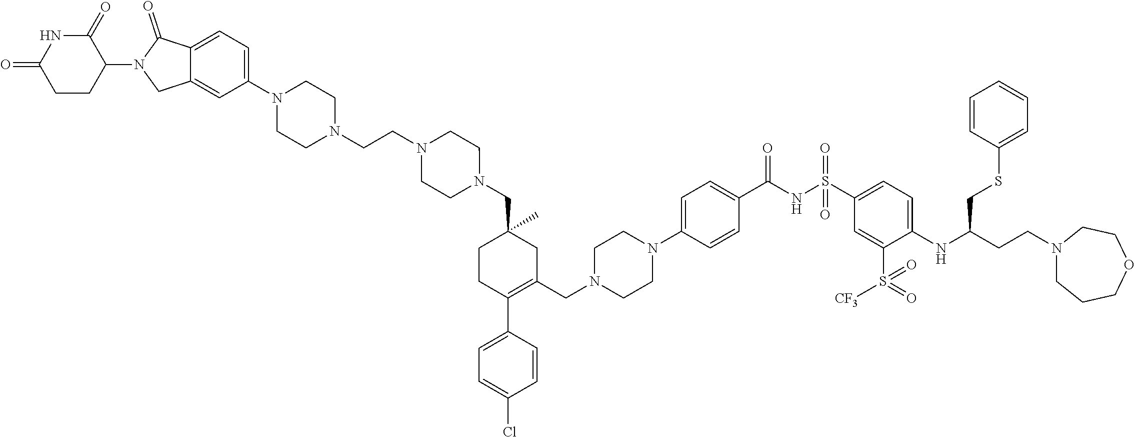 [SMILES: C[C@@]1(CN2CCN(CCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 64 | 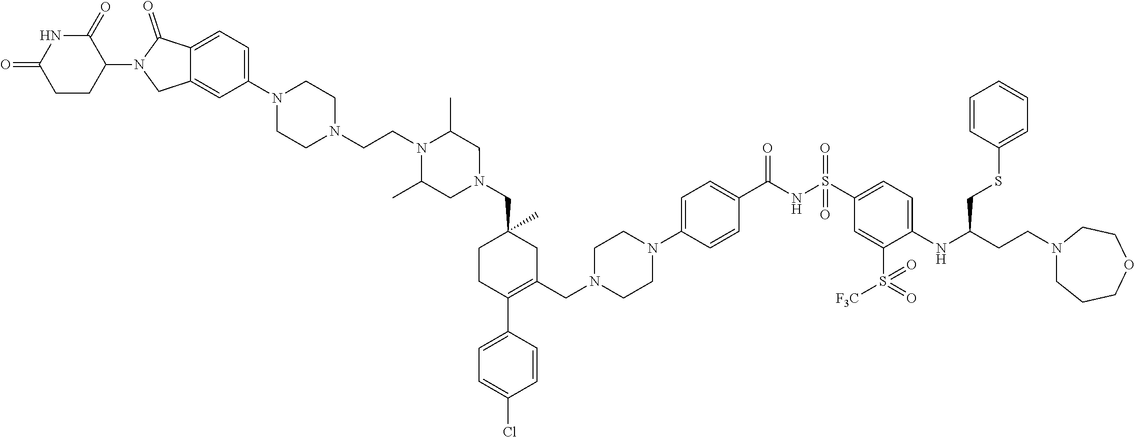 [SMILES: CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC(C)N1CCN1CCN(c2ccc3c(c2)CN(C2CCC(=O)NC2=O)C3=O)CC1] |
| 65 | 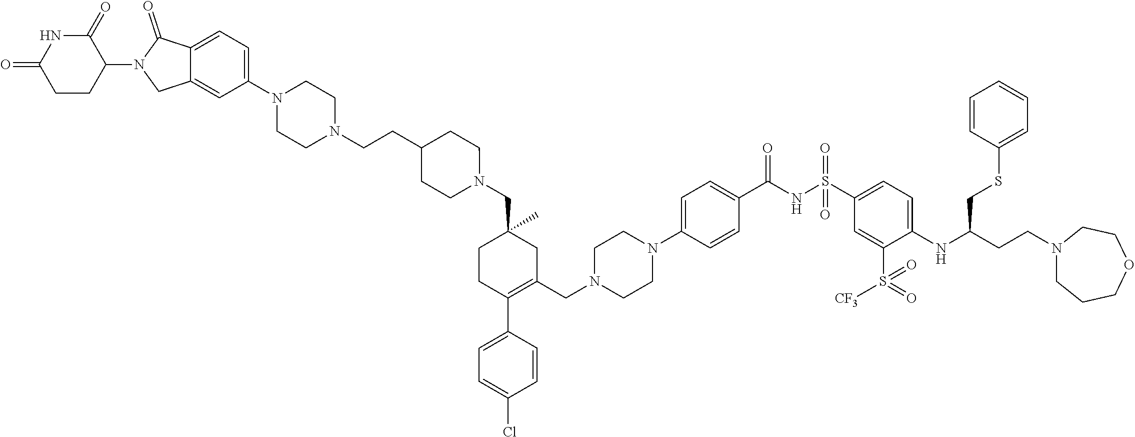 [SMILES: C[C@@]1(CN2CCC(CCN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 66 | 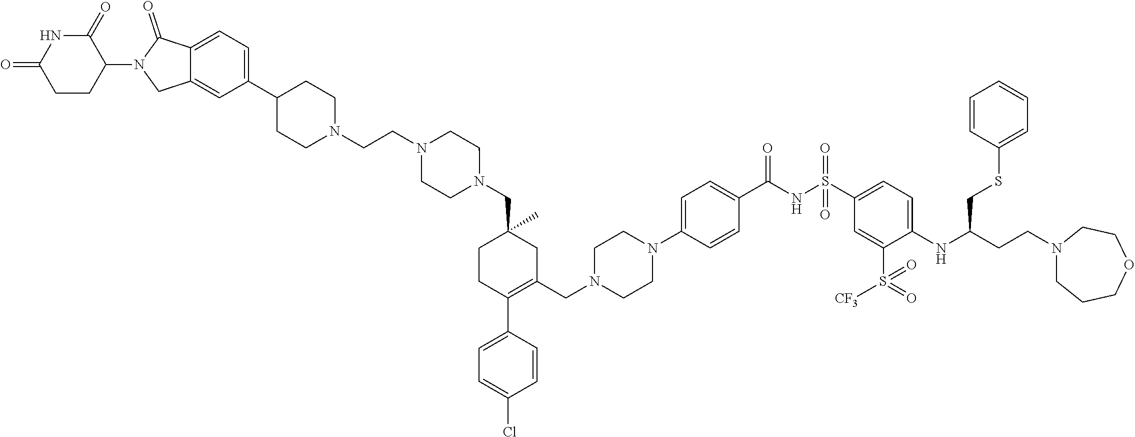 [SMILES: C[C@@]1(CN2CCN(CCN3CCC(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 67 | 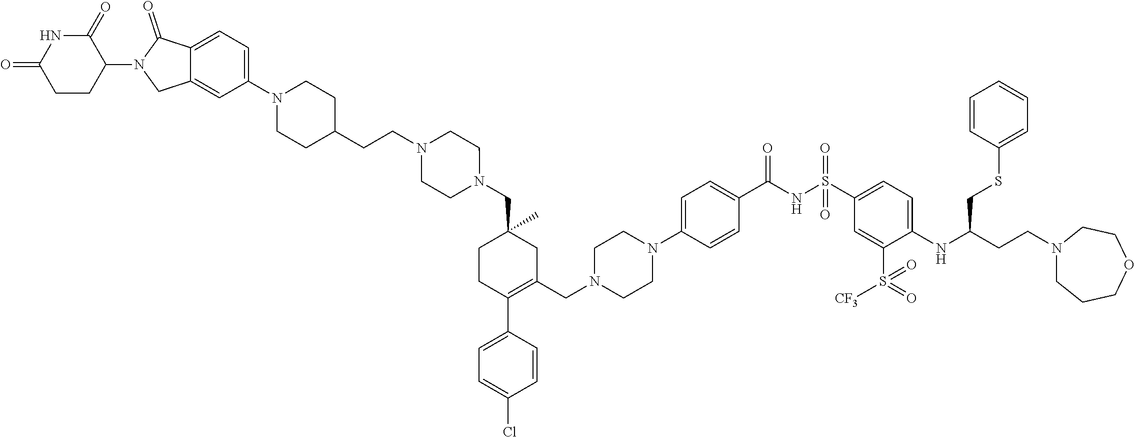 [SMILES: C[C@@]1(CN2CCN(CCC3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 68 | 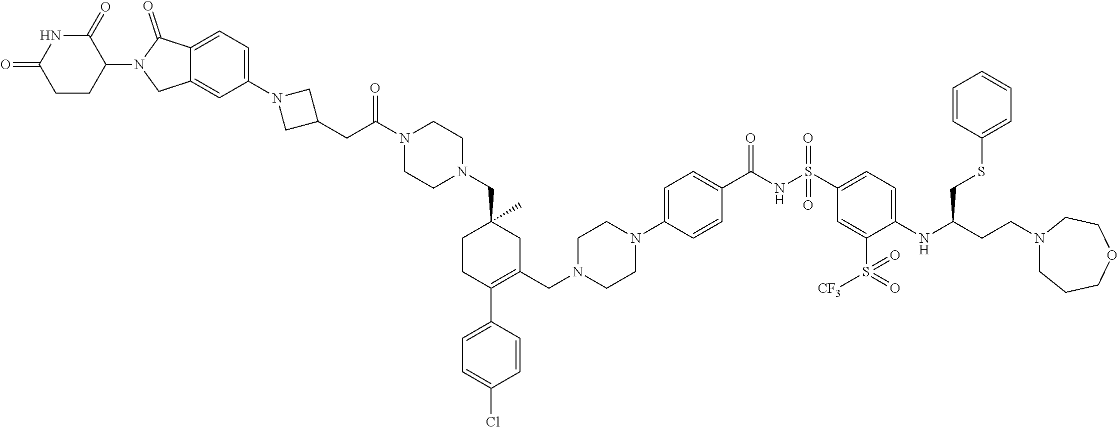 [SMILES: C[C@@]1(CN2CCN(C(=O)CC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 69 | 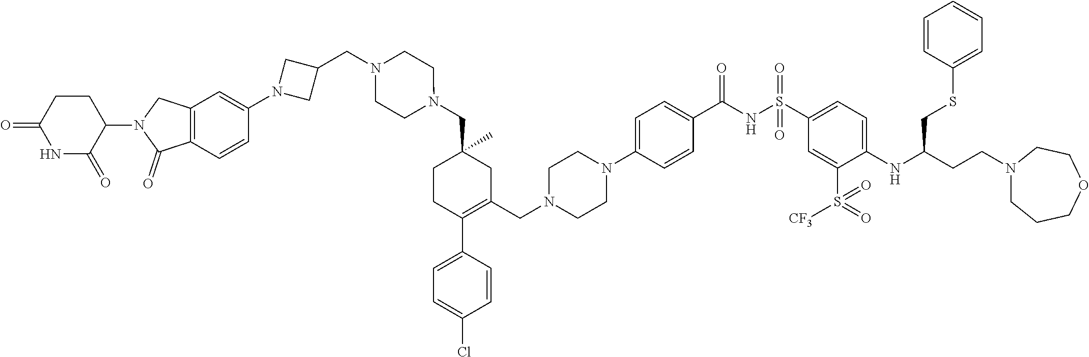 [SMILES: C[C@@]1(CN2CCN(CC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 70 | 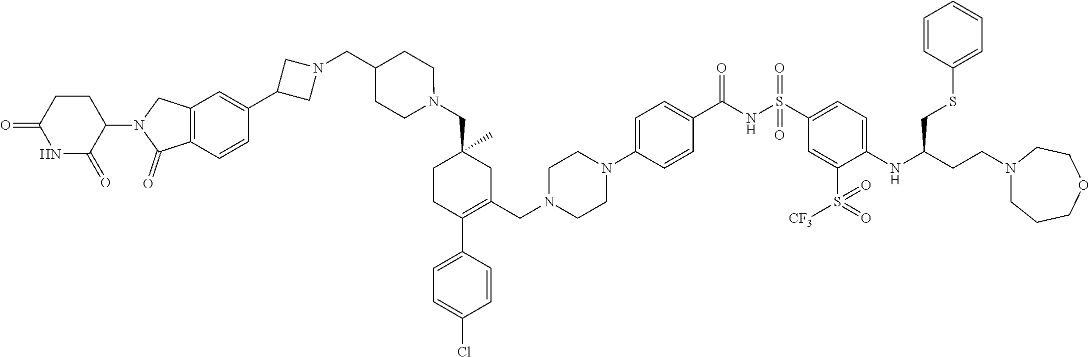 [SMILES: C[C@@]1(CN2CCC(CN3CC(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 71 | 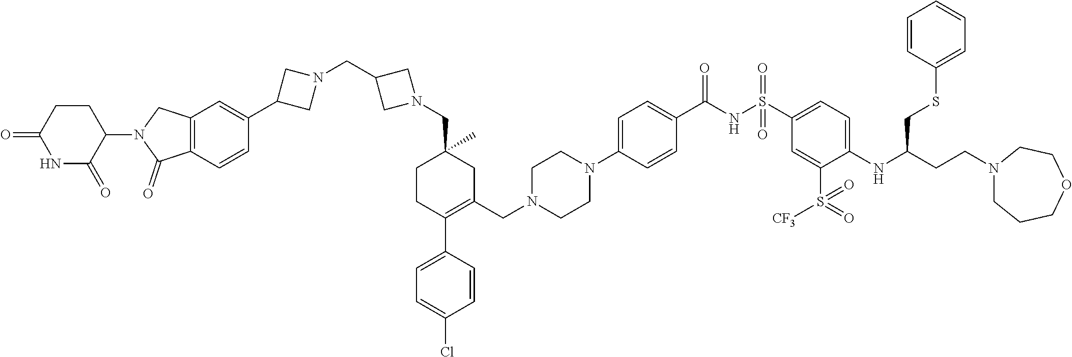 [SMILES: C[C@@]1(CN2CC(CN3CC(c4ccc5c(c4)CN(C4CCC(=O)NC4=O)C5=O)C3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 72 | 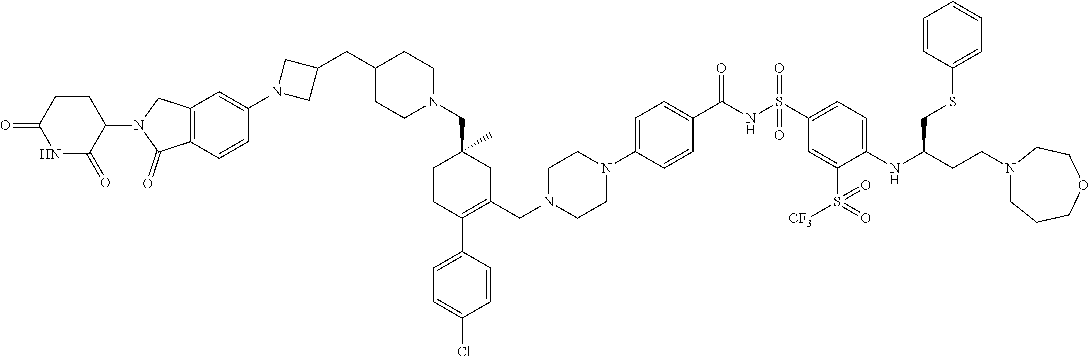 [SMILES: C[C@@]1(CN2CCC(CC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 73 | 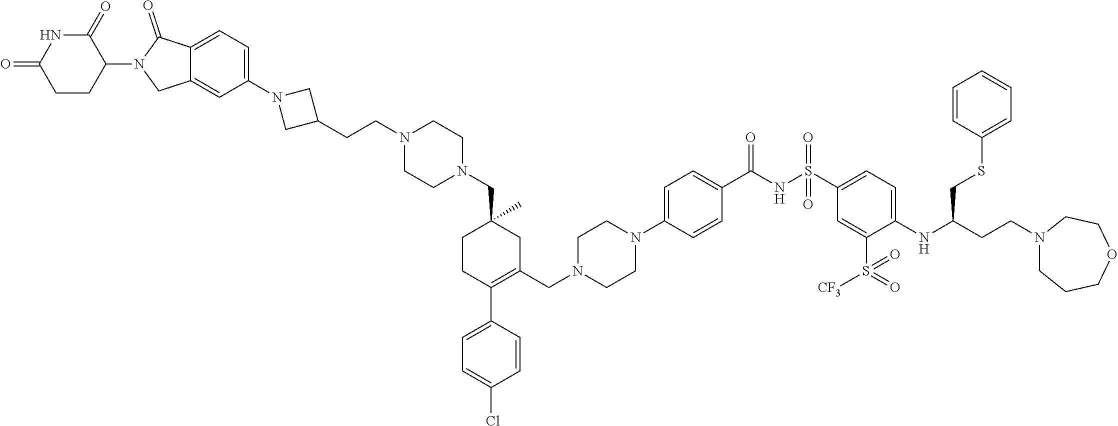 [SMILES: C[C@@]1(CN2CCN(CCC3CN(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 74 | 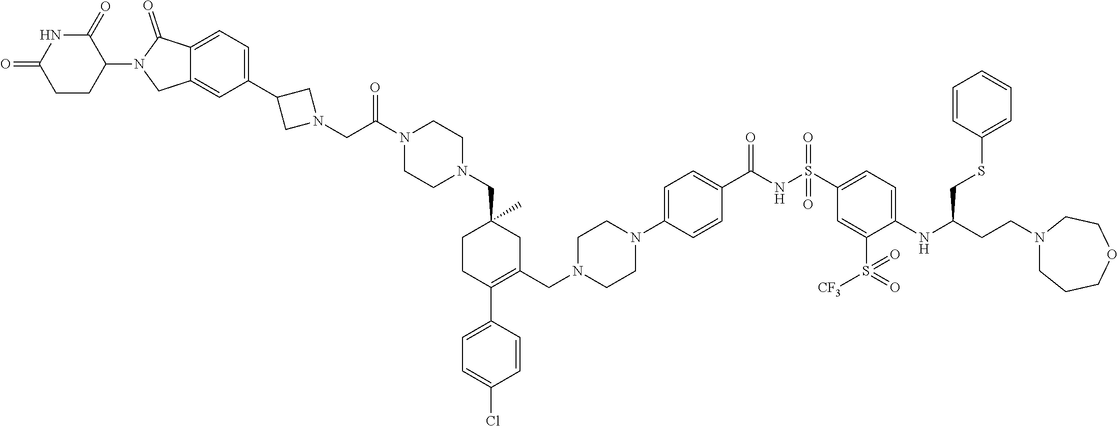 [SMILES: C[C@@]1(CN2CCN(C(=O)CN3CC(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 75 | 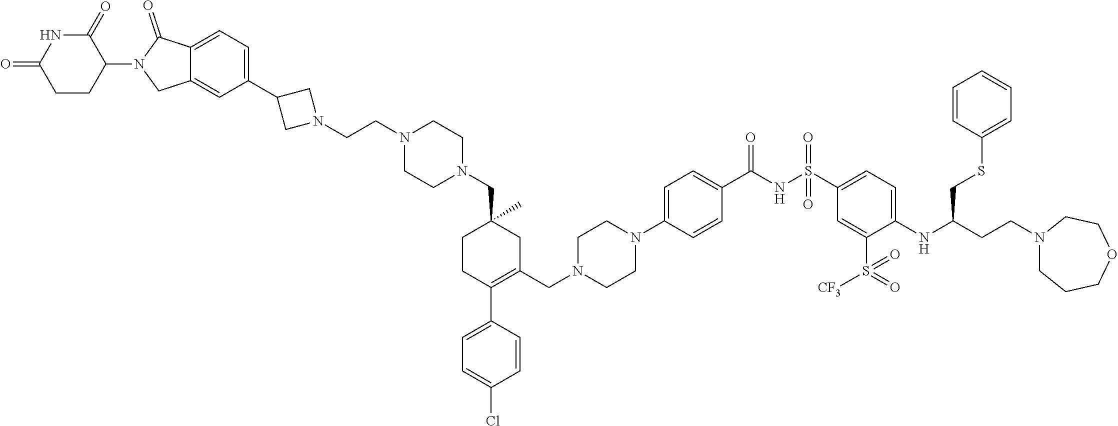 [SMILES: C[C@@]1(CN2CCN(CCN3CC(c4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 76 | 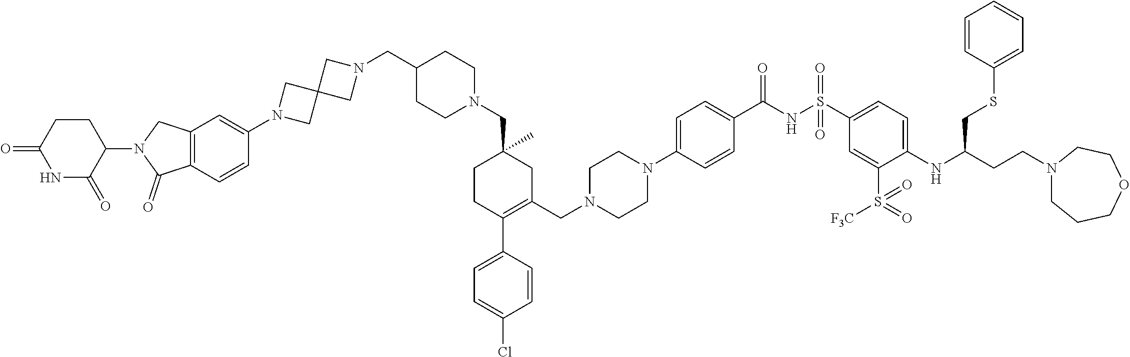 [SMILES: C[C@@]1(CN2CCC(CN3CC4(C3)CN(c3ccc5c(c3)CN(C3CCC(=O)NC3=O)C5=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 77 | 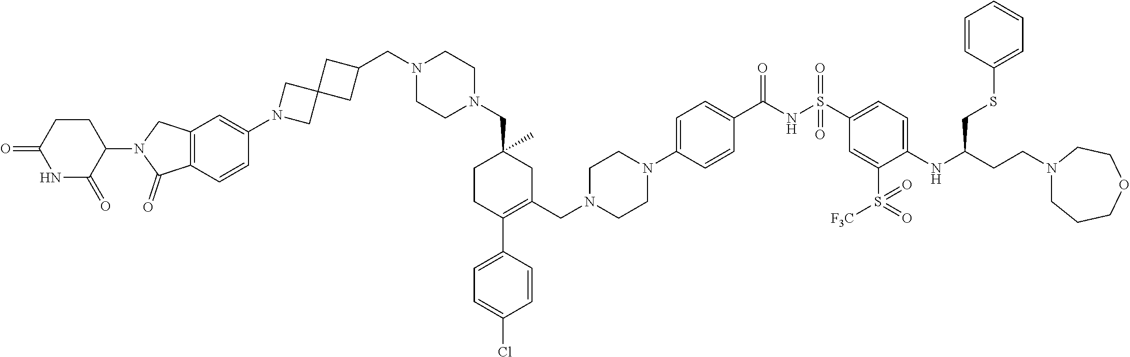 [SMILES: C[C@@]1(CN2CCN(CC3CC4(C3)CN(c3ccc5c(c3)CN(C3CCC(=O)NC3=O)C5=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 78 | 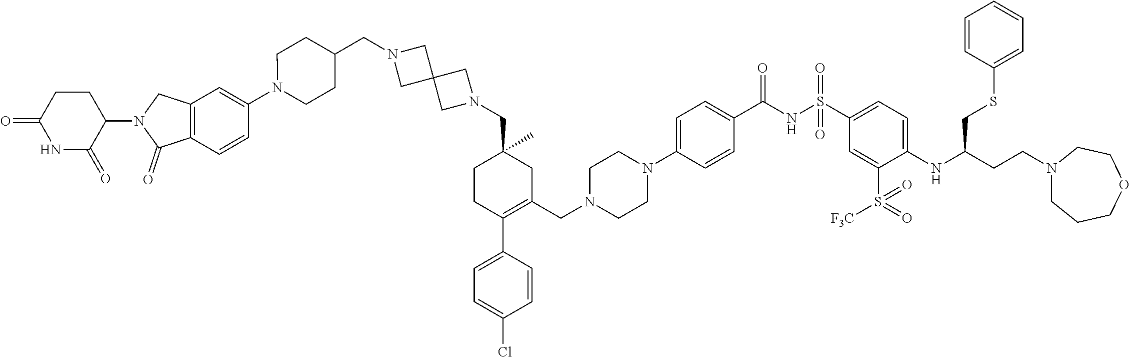 [SMILES: C[C@@]1(CN2CC3(CN(CC4CCN(c5ccc6c(c5)CN(C5CCC(=O)NC5=O)C6=O)CC4)C3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 79 | 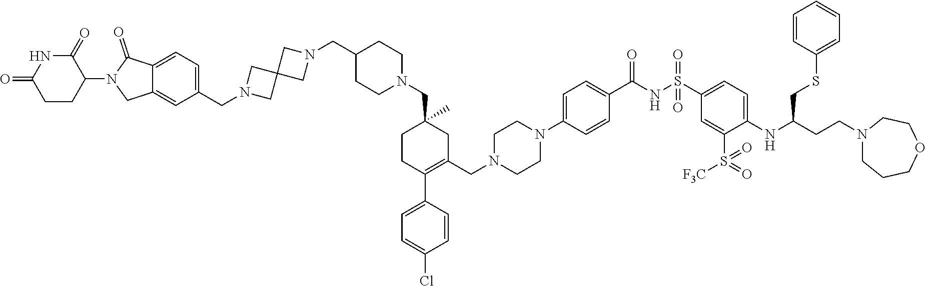 [SMILES: C[C@@]1(CN2CCC(CN3CC4(CN(Cc5ccc6c(c5)CN(C5CCC(=O)NC5=O)C6=O)C4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 80 | 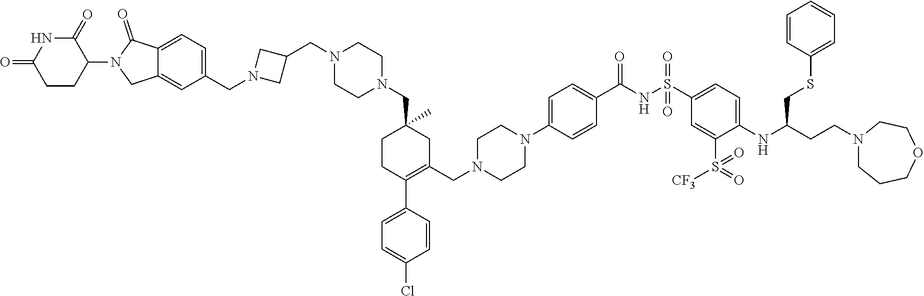 [SMILES: C[C@@]1(CN2CCN(CC3CN(Cc4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 81 | 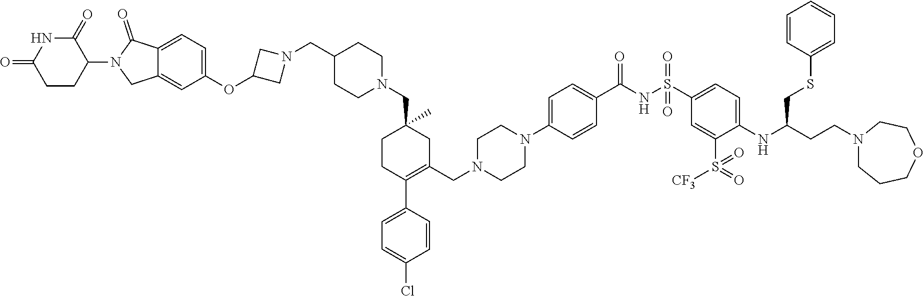 [SMILES: C[C@@]1(CN2CCC(CN3CC(Oc4ccc5c(c4)CN(C4CCC(=O)NC4=O)C5=O)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 82 | 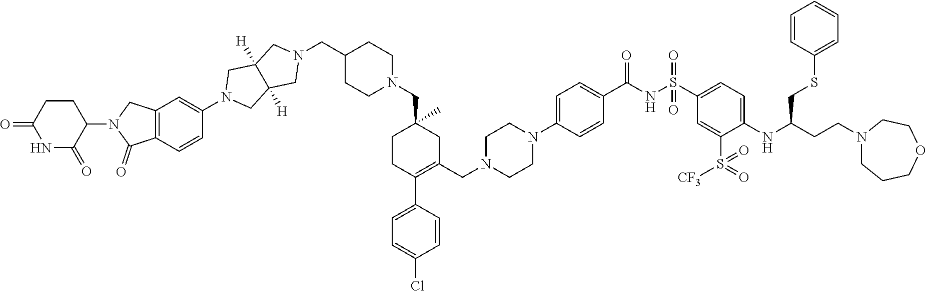 [SMILES: C[C@@]1(CN2CCC(CN3C[C@@H]4CN(c5ccc6c(c5)CN(C5CCC(=O)NC5=O)C6=O)C[C@@H]4C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 83 | 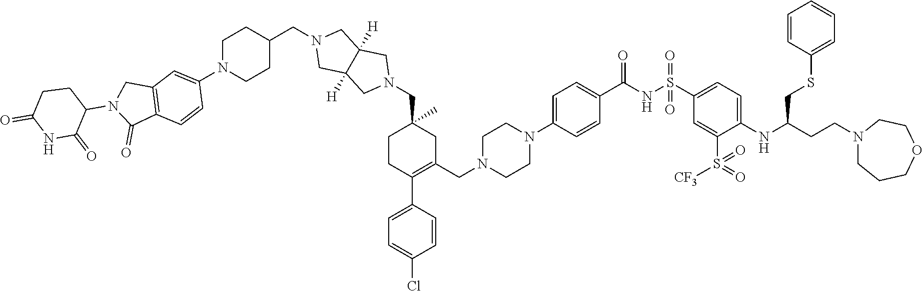 [SMILES: C[C@@]1(CN2C[C@H]3CN(CC4CCN(c5ccc6c(c5)CN(C5CCC(=O)NC5=O)C6=O)CC4)C[C@H]3C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 84 | 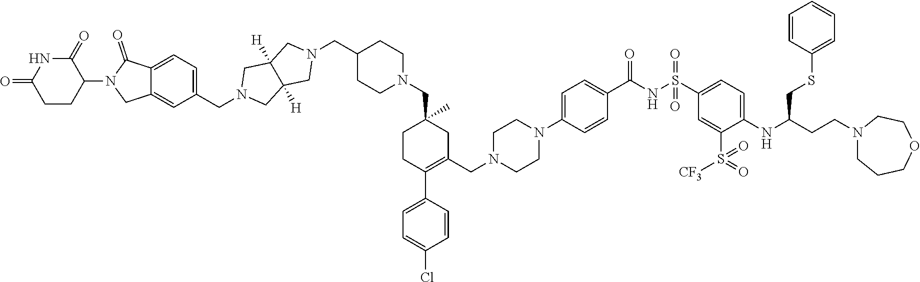 [SMILES: C[C@@]1(CN2CCC(CN3C[C@H]4CN(Cc5ccc6c(c5)CN(C5CCC(=O)NC5=O)C6=O)C[C@H]4C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 85 | 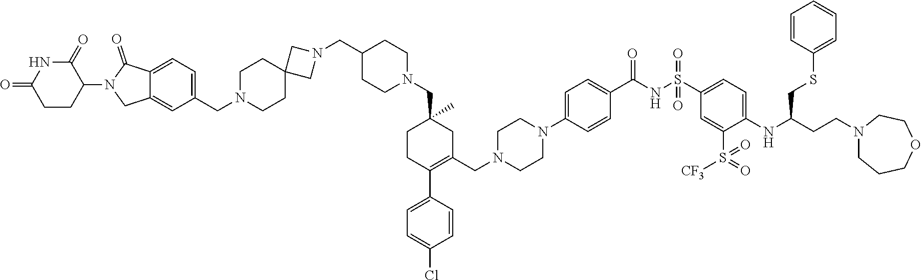 [SMILES: C[C@@]1(CN2CCC(CN3CC4(CCN(Cc5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 86 | 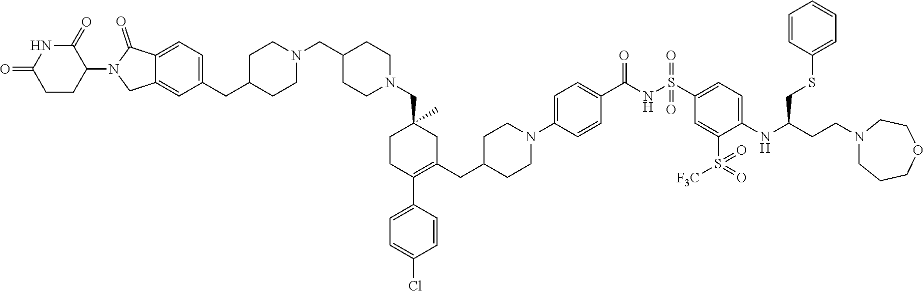 [SMILES: C[C@@]1(CN2CCC(CN3CCC(Cc4ccc5c(c4)CN(C4CCC(=O)NC4=O)C5=O)CC3)CC2)CCC(c2ccc(Cl)cc2)=C(CC2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 87 | 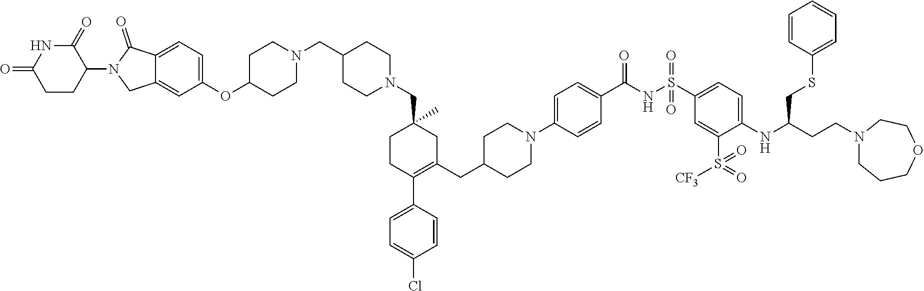 [SMILES: C[C@@]1(CN2CCC(CN3CCC(Oc4ccc5c(c4)CN(C4CCC(=O)NC4=O)C5=O)CC3)CC2)CCC(c2ccc(Cl)cc2)=C(CC2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 88 | 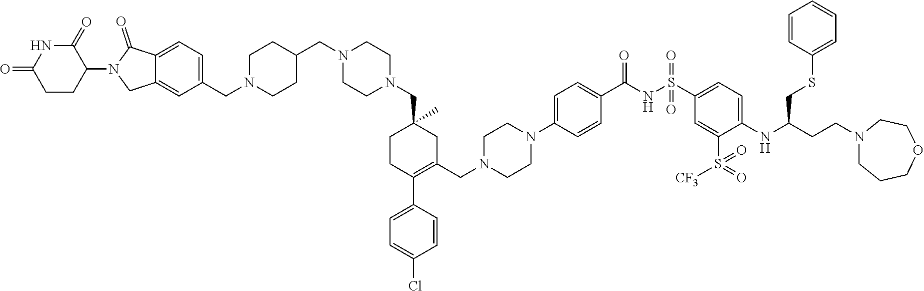 [SMILES: C[C@@]1(CN2CCN(CC3CCN(Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 89 | 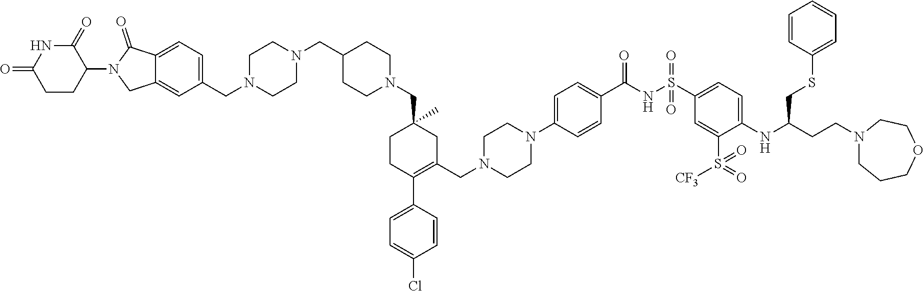 [SMILES: C[C@@]1(CN2CCC(CN3CCN(Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 90 | 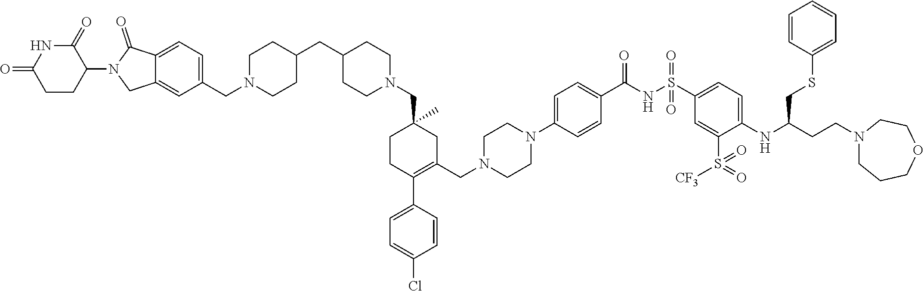 [SMILES: C[C@@]1(CN2CCC(CC3CCN(Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 91 | 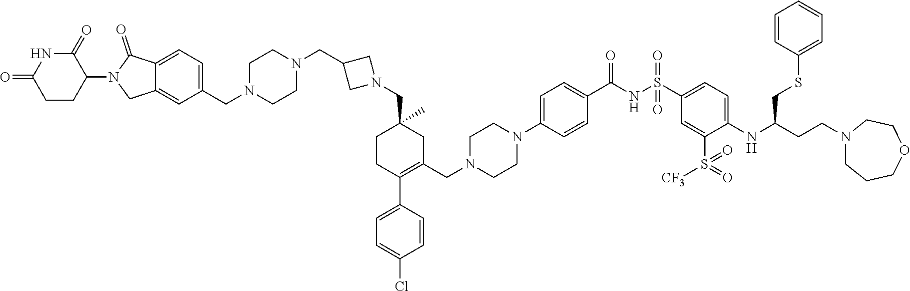 [SMILES: C[C@@]1(CN2CC(CN3CCN(Cc4ccc5c(c4)CN(C4CCC(=O)NC4=O)C5=O)CC3)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 92 | 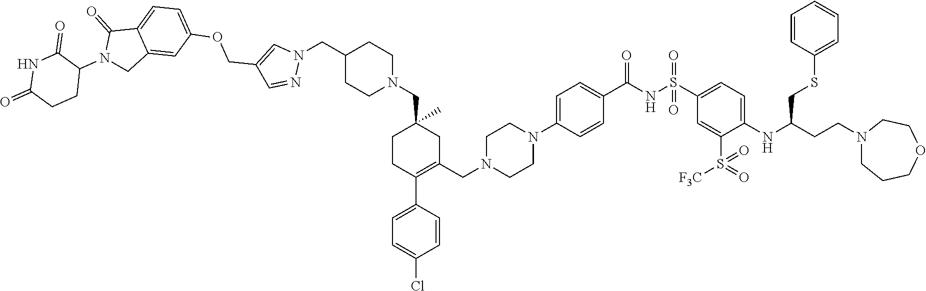 [SMILES: C[C@@]1(CN2CCC(Cn3cc(COc4ccc5c(c4)CN(C4CCC(=O)NC4=O)C5=O)cn3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 93 | 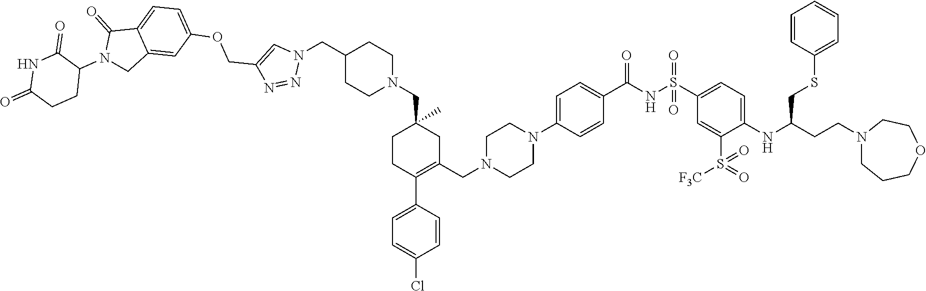 [SMILES: C[C@@]1(CN2CCC(Cn3cc(COc4ccc5c(c4)CN(C4CCC(=O)NC4=O)C5=O)nn3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 94 | 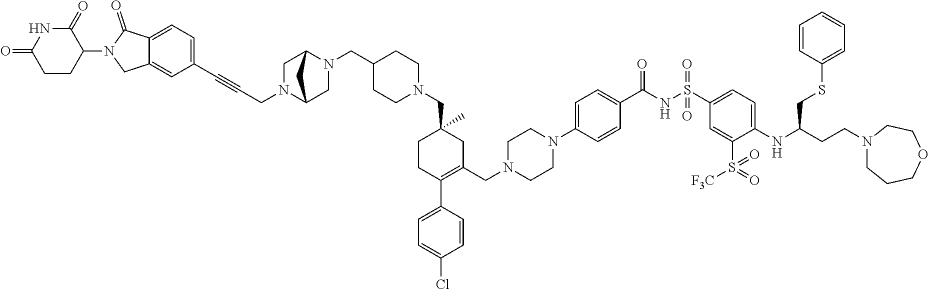 [SMILES: C[C@@]1(CN2CCC(CN3C[C@H]4C[C@@H]3CN4CC#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 95 | 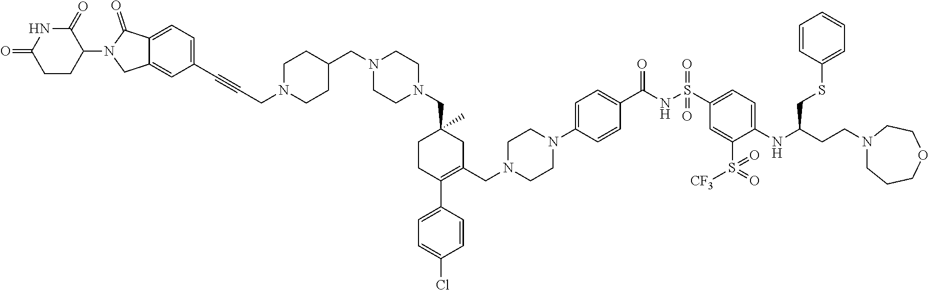 [SMILES: C[C@@]1(CN2CCN(CC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 96 | 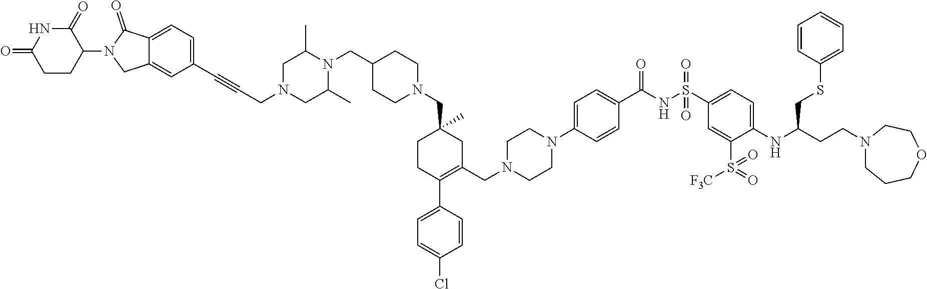 [SMILES: CC1CN(CC#Cc2ccc3c(c2)CN(C2CCC(=O)NC2=O)C3=O)CC(C)N1CC1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 97 | 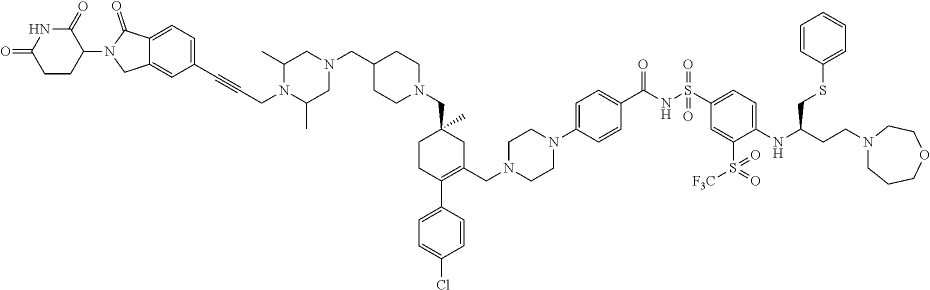 [SMILES: CC1CN(CC2CCN(C[C@]3(C)CCC(c4ccc(Cl)cc4)=C(CN4CCN(c5ccc(C(=O)NS(=O)(=O)c6ccc(N[C@H](CCN7CCCOCC7)CSc7ccccc7)c(S(=O)(=O)C(F)(F)F)c6)cc5)CC4)C3)CC2)CC(C)N1CC#Cc1ccc2c(c1)CN(C1CCC(=O)NC1=O)C2=O] |
| 98 | 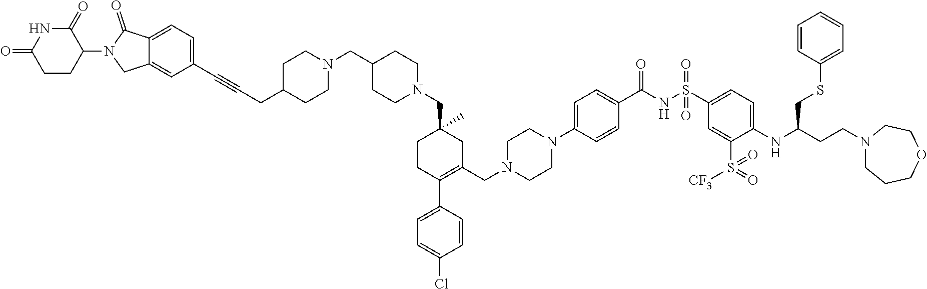 [SMILES: C[C@@]1(CN2CCC(CN3CCC(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 99 | 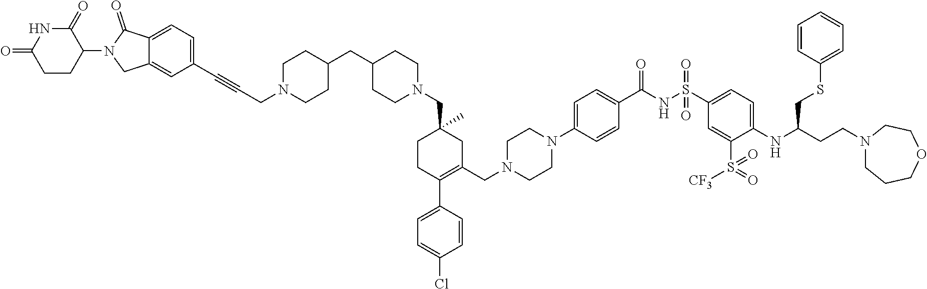 [SMILES: C[C@@]1(CN2CCC(CC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 100 | 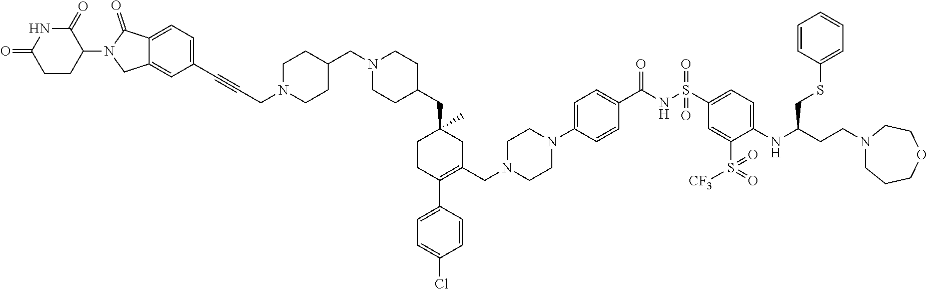 [SMILES: C[C@@]1(CC2CCN(CC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 101 | 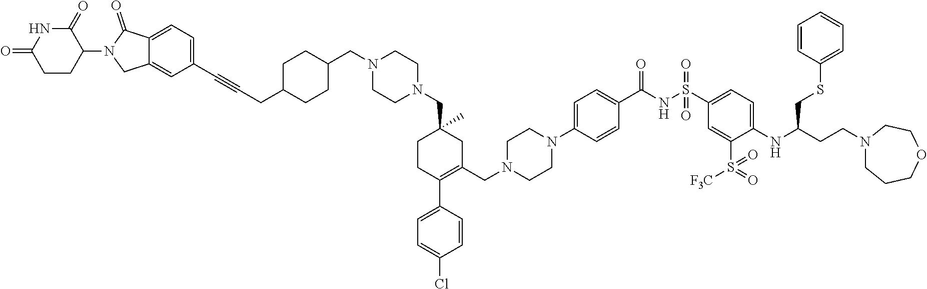 [SMILES: C[C@@]1(CN2CCN(CC3CCC(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 102 | 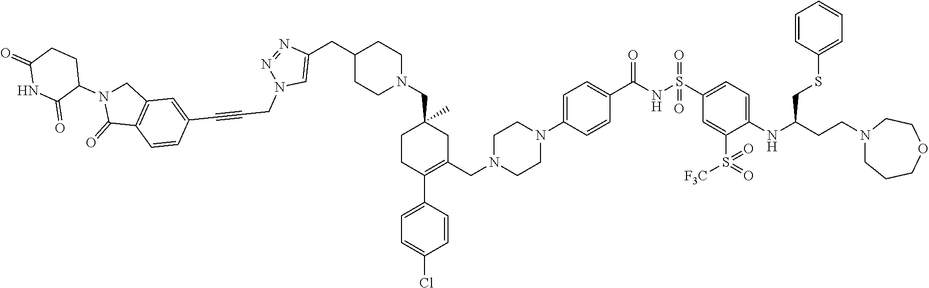 [SMILES: C[C@@]1(CN2CCC(Cc3cn(CC#Cc4ccc5c(c4)CN(C4CCC(=O)NC4=O)C5=O)nn3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 103 | 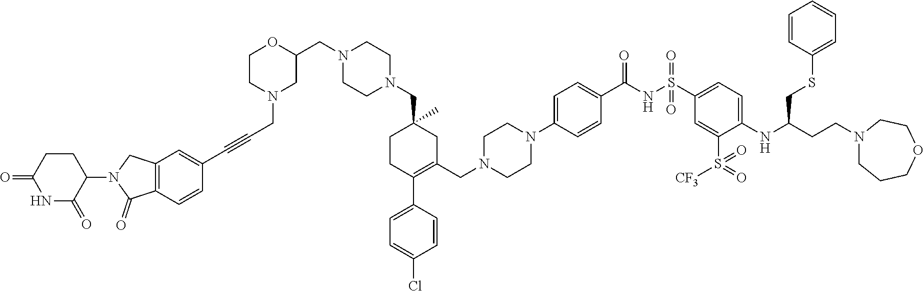 [SMILES: C[C@@]1(CN2CCN(CC3CN(CC#Cc4ccc5c(c4)CN(C4CCC(=O)NC4=O)C5=O)CCO3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 104 | 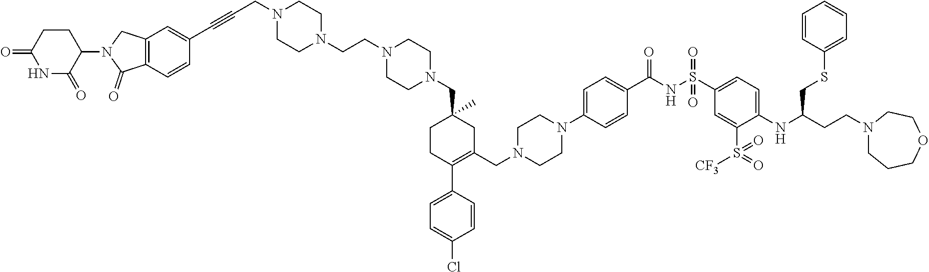 [SMILES: C[C@@]1(CN2CCN(CCN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 105 | 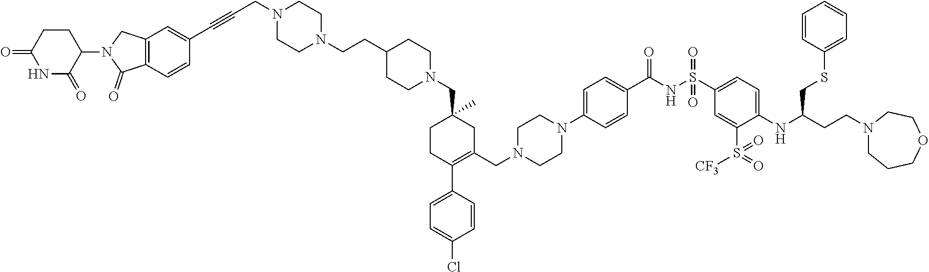 [SMILES: C[C@@]1(CN2CCC(CCN3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 106 | 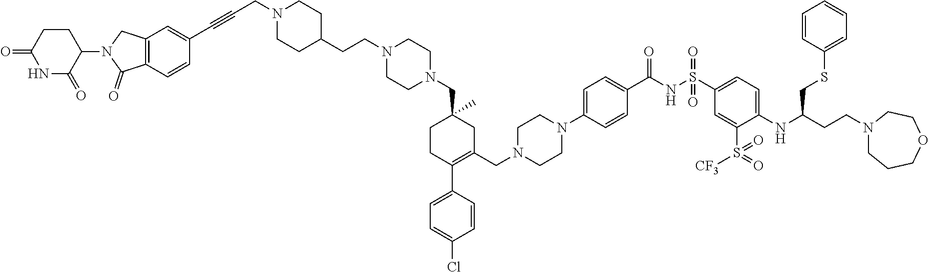 [SMILES: C[C@@]1(CN2CCN(CCC3CCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 107 | 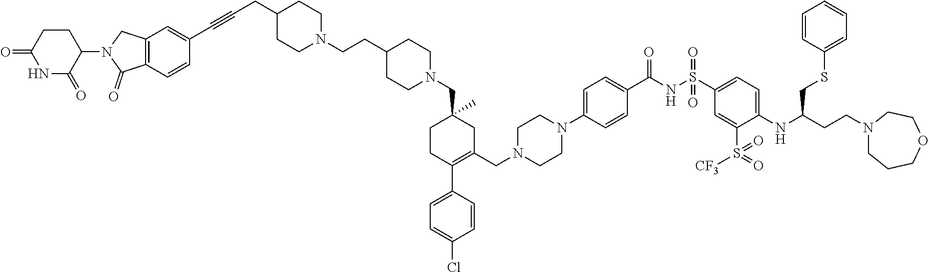 [SMILES: C[C@@]1(CN2CCC(CCN3CCC(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 108 | 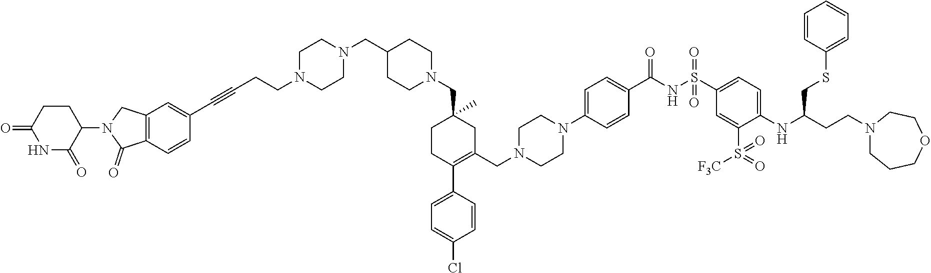 [SMILES: C[C@@]1(CN2CCC(CN3CCN(CCC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 109 | 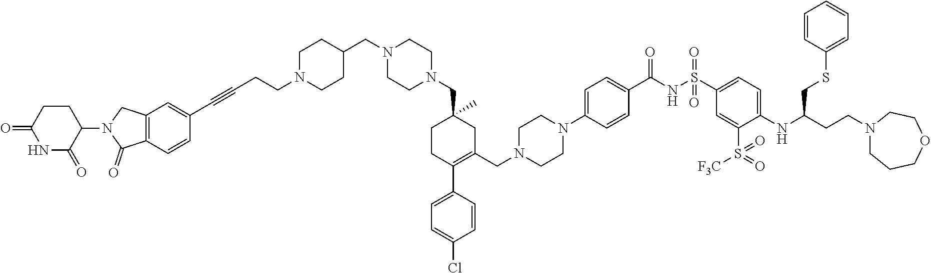 [SMILES: C[C@@]1(CN2CCN(CC3CCN(CCC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 110 | 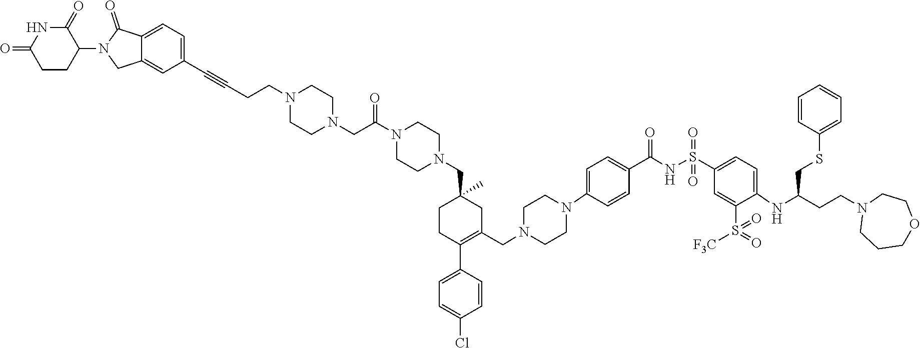 [SMILES: C[C@@]1(CN2CCN(C(=O)CN3CCN(CCC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 111 | 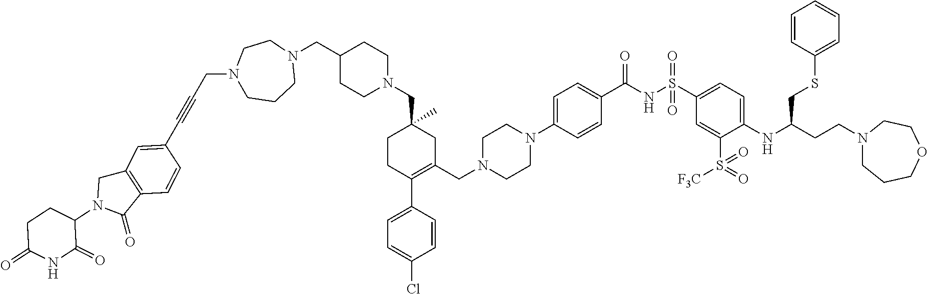 [SMILES: C[C@@]1(CN2CCC(CN3CCCN(CC#Cc4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 112 | 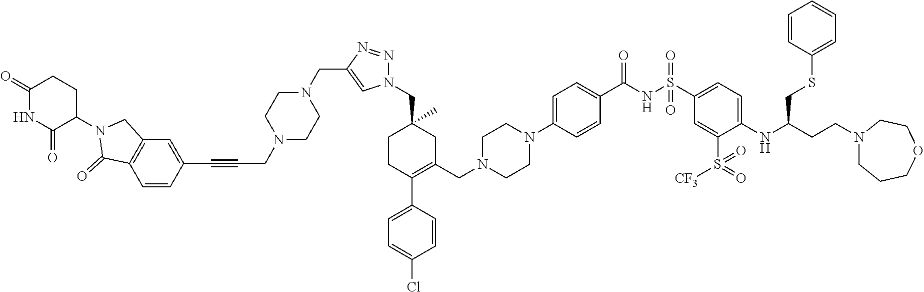 [SMILES: C[C@@]1(Cn2cc(CN3CCN(CC#Cc4ccc5c(c4)CN(C4CCC(=O)NC4=O)C5=O)CC3)nn2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 113 | 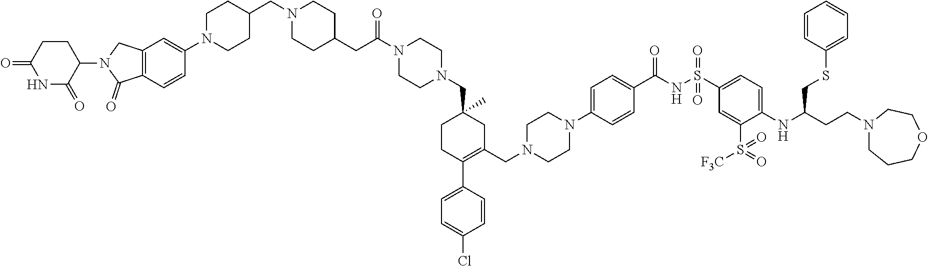 [SMILES: C[C@@]1(CN2CCN(C(=O)CC3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 114 | 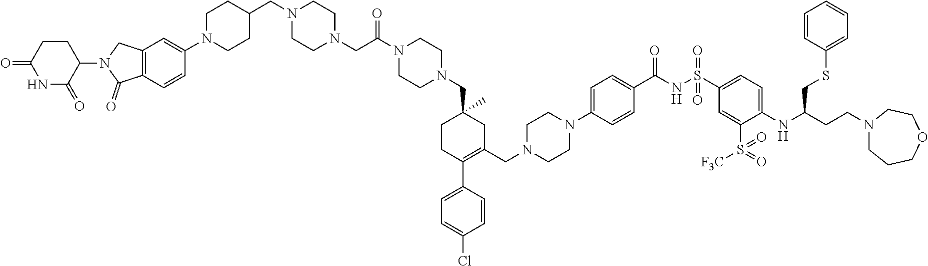 [SMILES: C[C@@]1(CN2CCN(C(=O)CN3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 115 | 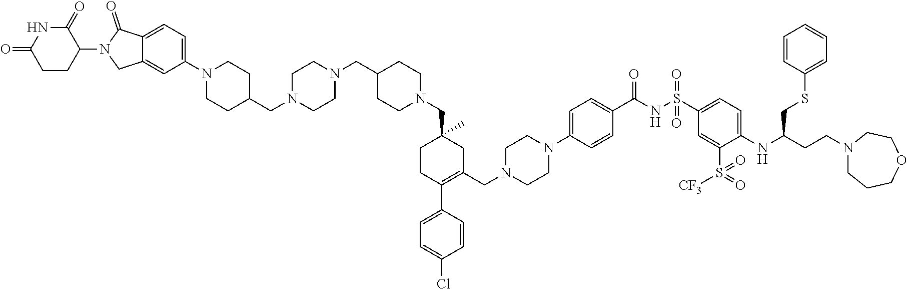 [SMILES: C[C@@]1(CN2CCC(CN3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 116 | 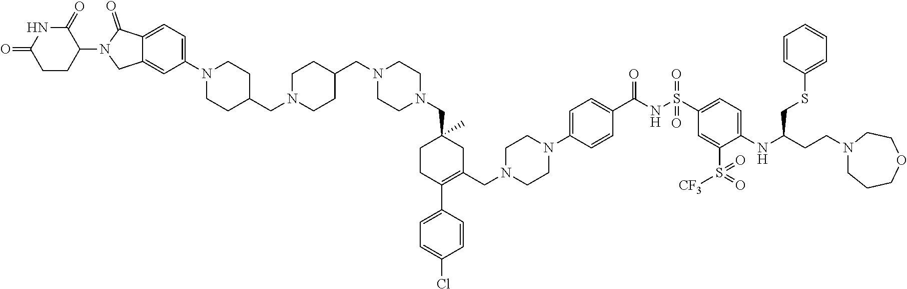 [SMILES: C[C@@]1(CN2CCN(CC3CCN(CC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 117 | 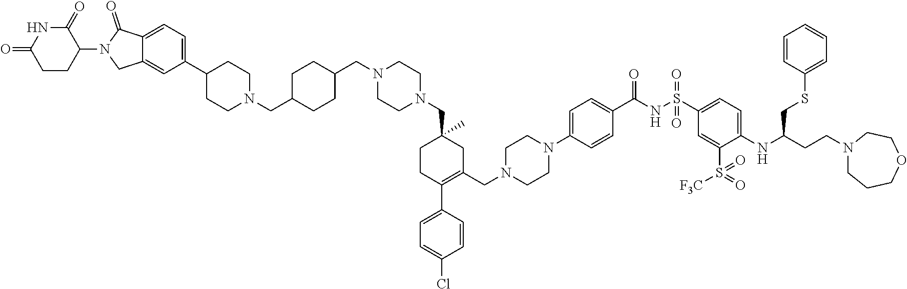 [SMILES: C[C@@]1(CN2CCN(CC3CCC(CN4CCC(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 118 | 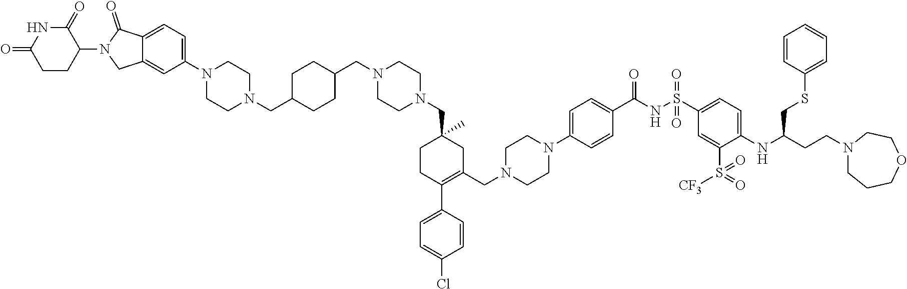 [SMILES: C[C@@]1(CN2CCN(CC3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 119 | 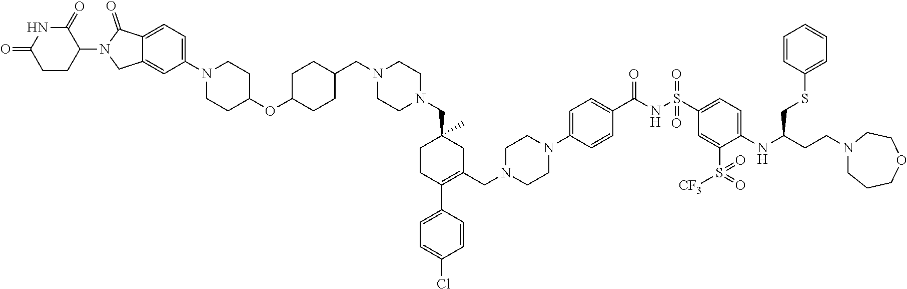 [SMILES: C[C@@]1(CN2CCN(CC3CCC(OC4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 120 | 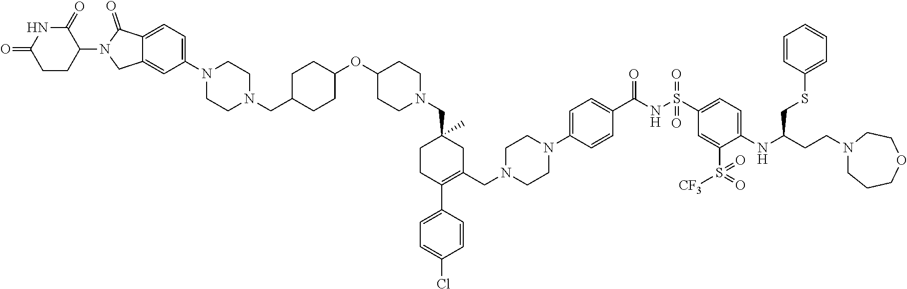 [SMILES: C[C@@]1(CN2CCC(OC3CCC(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 121 | 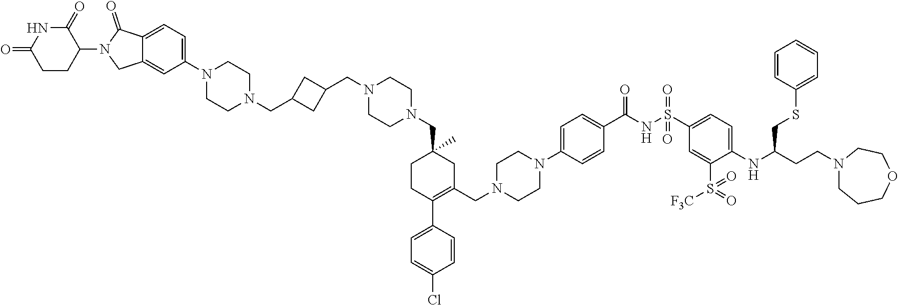 [SMILES: C[C@@]1(CN2CCN(CC3CC(CN4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 122 | 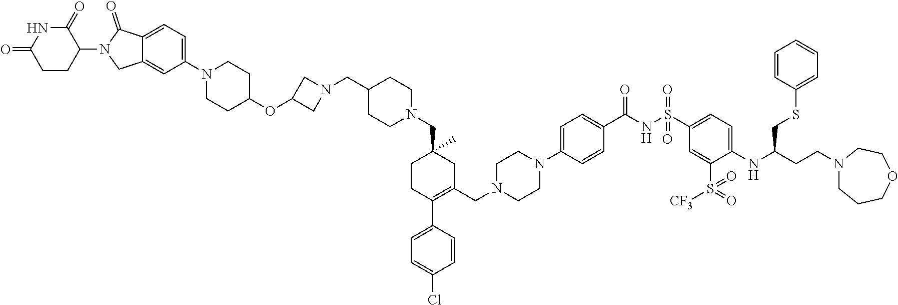 [SMILES: C[C@@]1(CN2CCC(CN3CC(OC4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 123 | 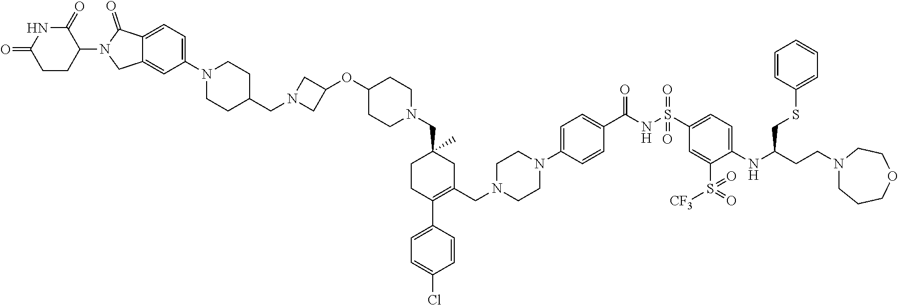 [SMILES: C[C@@]1(CN2CCC(OC3CN(CC4CCN(c5ccc6c(c5)CN(C5CCC(=O)NC5=O)C6=O)CC4)C3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 124 | 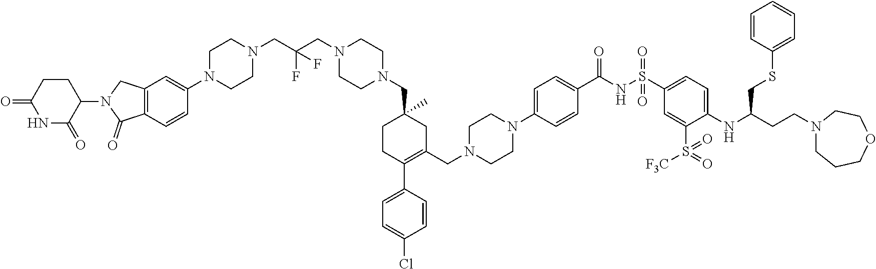 [SMILES: C[C@@]1(CN2CCN(CC(F)(F)CN3CCN(c4ccc5c(c4)CN(C4CCC(=O)NC4=O)C5=O)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 125 | 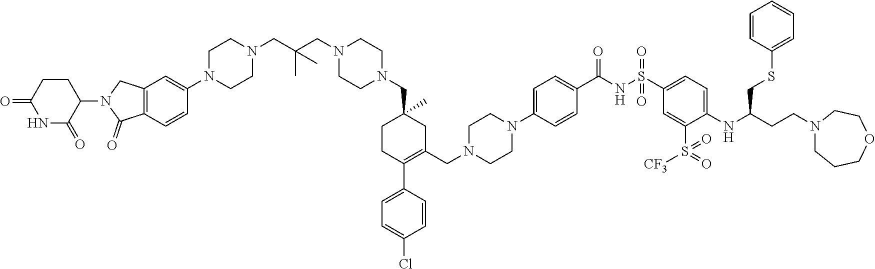 [SMILES: CC(C)(CN1CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1)CN1CCN(c2ccc3c(c2)CN(C2CCC(=O)NC2=O)C3=O)CC1] |
| 126 | 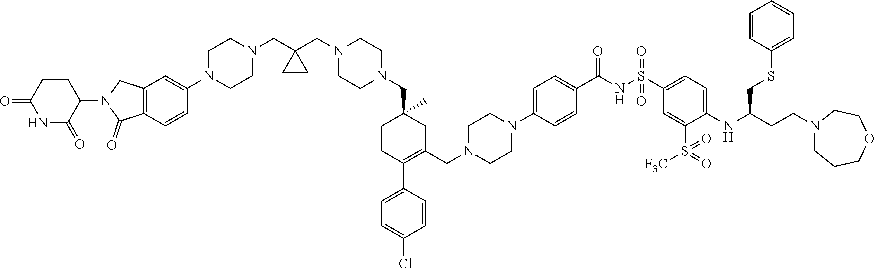 [SMILES: C[C@@]1(CN2CCN(CC3(CN4CCN(c5ccc6c(c5)CN(C5CCC(=O)NC5=O)C6=O)CC4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 127 | 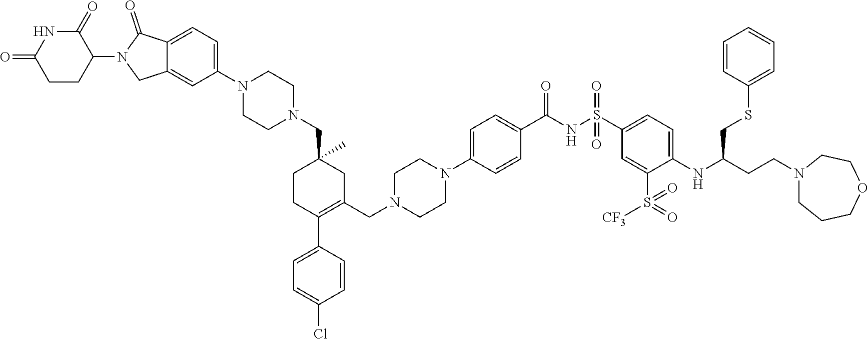 [SMILES: C[C@@]1(CN2CCN(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 128 | 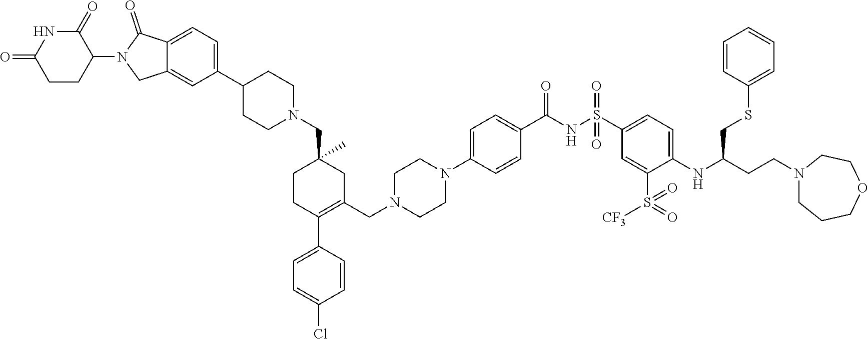 [SMILES: C[C@@]1(CN2CCC(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |

64. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

| Com- | |
|---|---|
| pound | |
| Number | Formula |
| 129 | 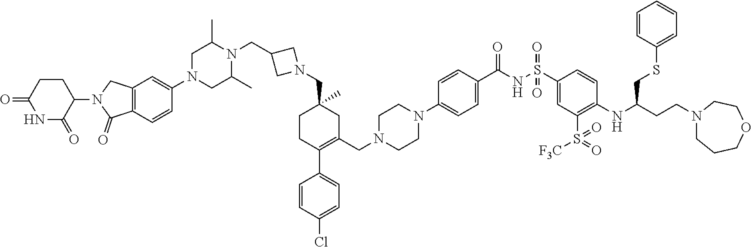 [SMILES: CC1CN(c2ccc3c(c2)CN(C2CCC(=O)NC2=O)C3=O)CC(C)N1CC1CN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)C1] |
| 130 | 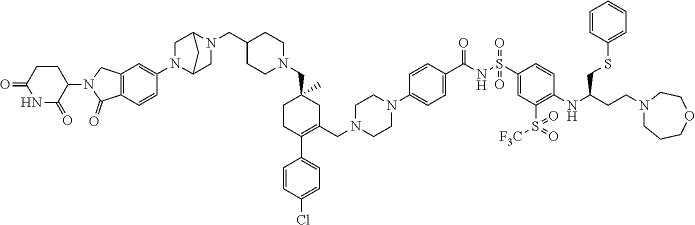 [SMILES: C[C@@]1(CN2CCC(CN3CC4CC3CN4c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 131 | 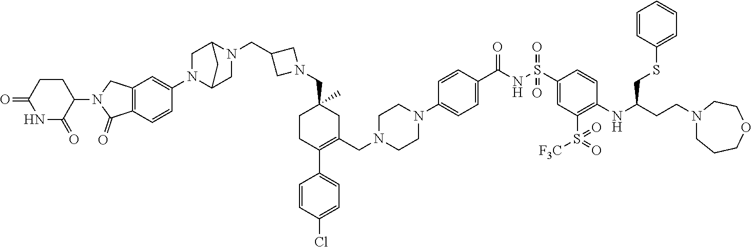 [SMILES: C[C@@]1(CN2CC(CN3CC4CC3CN4c3ccc4c(c3)CN(C3CCC(=O)NC3=O)C4=O)C2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 132 | 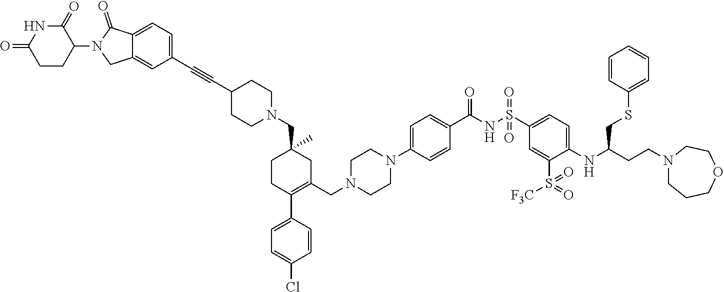 [SMILES: C[C@@]1(CN2CCC(C#Cc3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 133 | 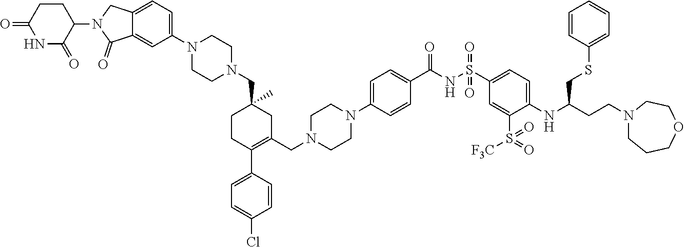 [SMILES: C[C@@]1(CN2CCN(c3ccc4c(c3)C(=O)N(C3CCC(=O)NC3=O)C4)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 134 | 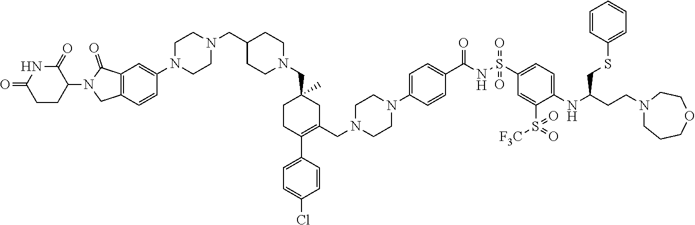 [SMILES: C[C@@]1(CN2CCC(CN3CCN(c4ccc5c(c4)C(=O)N(C4CCC(=O)NC4=O)C5)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 135 | 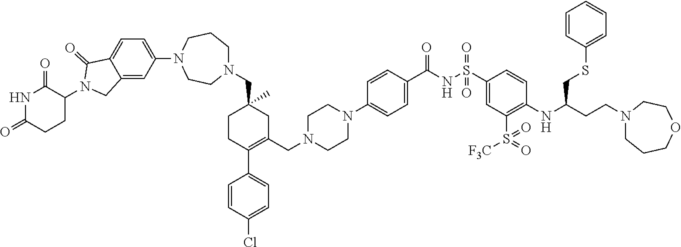 [SMILES: C[C@@]1(CN2CCCN(c3ccc4c(c3)CN(C3CCC(=O)NC3=O)C4=O)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 136 | 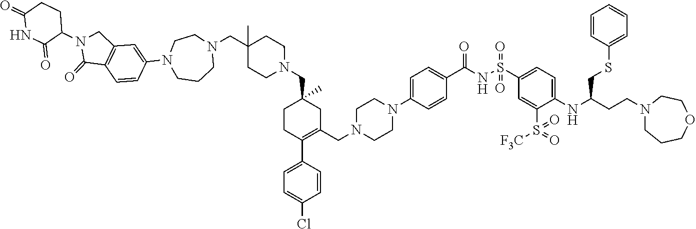 [SMILES: CC1(CN2CCCN(c3ccc4c(c3)CN(C3CCC(=O)NC3=O)C4=O)CC2)CCN(C[C@]2(C)CCC(c3ccc(Cl)cc3)=C(CN3CCN(c4ccc(C(=O)NS(=O)(=O)c5ccc(N[C@H](CCN6CCCOCC6)CSc6ccccc6)c(S(=O)(=O)C(F)(F)F)c5)cc4)CC3)C2)CC1] |
| 137 | 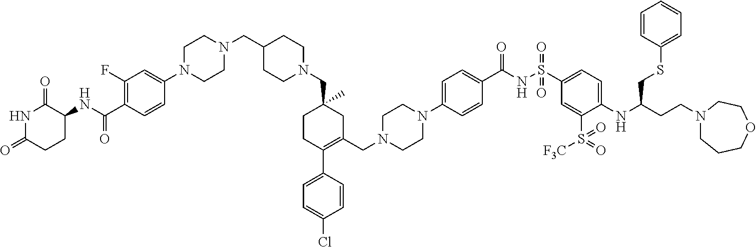 [SMILES: C[C@@]1(CN2CCC(CN3CCN(c4ccc(C(=O)N[C@H]5CCC(=O)NC5=O)c(F)c4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 138 | 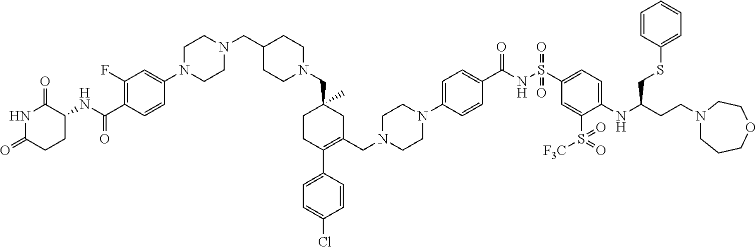 [SMILES: C[C@@]1(CN2CCC(CN3CCN(c4ccc(C(=O)N[C@@H]5CCC(=O)NC5=O)c(F)c4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 139 | 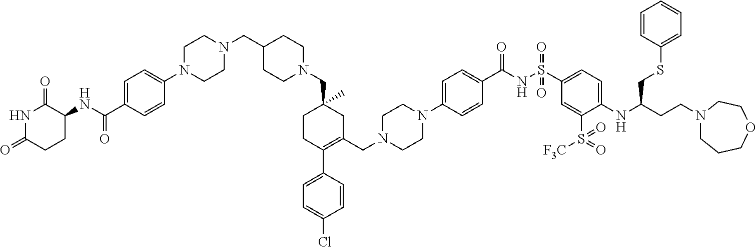 [SMILES: C[C@@]1(CN2CCC(CN3CCN(c4ccc(C(=O)N[C@H]5CCC(=O)NC5=O)cc4)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 140 | 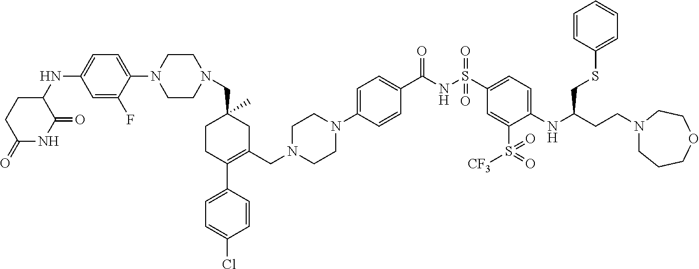 [SMILES: C[C@@]1(CN2CCN(c3ccc(NC4CCC(=O)NC4=O)cc3F)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |
| 141 | 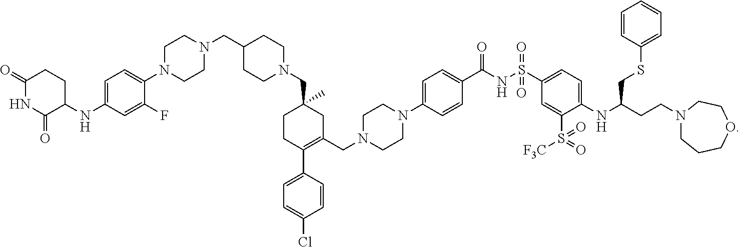 [SMILES: C[C@@]1(CN2CCC(CN3CCN(c4ccc(NC5CCC(=O)NC5=O)cc4F)CC3)CC2)CCC(c2ccc(Cl)cc2)=C(CN2CCN(c3ccc(C(=O)NS(=O)(=O)c4ccc(N[C@H](CCN5CCCOCC5)CSc5ccccc5)c(S(=O)(=O)C(F)(F)F)c4)cc3)CC2)C1] |

65. The compound of any one of claims 1-64, or a pharmaceutically acceptable salt thereof.

66. A pharmaceutical composition comprising a compound of any one of claims 1-64, or a pharmaceutically acceptable salt, solvate, hydrate, polymorph, co-crystal, tautomer, stereoisomer, isotopically labeled compound, or prodrug thereof, and a pharmaceutically acceptable carrier.

67. The pharmaceutical composition of claim 66 further comprising an additional pharmaceutical agent.

68. The pharmaceutical composition of claim 67, wherein the additional pharmaceutical agent is an anti-cancer agent.

69. The pharmaceutical composition of claim 68, wherein the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

70. A kit comprising:
a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69; and
instructions for using the compound, or a pharmaceutically acceptable salt, solvate, hydrate, polymorph, co-crystal, tautomer, stereoisomer, isotopically labeled compound, or prodrug thereof, or the pharmaceutical composition.

71. A method of degrading a Bcl-2 protein in a cell, tissue, or biological sample, the method comprising contacting the cell, tissue, or biological sample with an effective amount of a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69.

72. A method of degrading a Bcl-xL protein in a cell, tissue, or biological sample, the method comprising contacting the cell, tissue, or biological sample with an effective amount of a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69.

73. The method of any one of claims 71-72, wherein the cell is a cancer cell.

74. A method of selectively killing one or more cancer cells in a tissue or biological sample comprising contacting the tissue or biological sample with an effective amount of a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69.

75. The method of any one of claims 71-74, wherein the cell, tissue, biological sample, or cancer cell is in vitro.

76. The method of any one of claims 71-74, wherein the cell, tissue, biological sample, or cancer cell is in a subject.

77. A method of treating a disease in a subject in need thereof, the method comprising administering to the subject an effective amount of a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69.

78. A method of preventing a disease in a subject in need thereof, the method comprising administering to the subject an effective amount of a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69.

79. The method of any one of claims 77-78, wherein the disease is an age-related disease.

80. The method of any one of claims 77-79, wherein the disease is an age-related cognitive dysfunction, age-related intervertebral disc disease, age-related muscle loss, Alzheimer's disease, amyotrophic lateral sclerosis, ataxia, AV fistulae, bone marrow transplant complication, cardiac dysfunction, cataracts, chemotherapy complication, chronic obstructive, cirrhosis, COVID-19 complication, diabetes, frailty, glaucoma, hepatic steatosis, hyperoxic lung damage, idiopathic pulmonary fibrosis, macular degeneration, MGUS, myeloma, obesity, obesity-related neuropsychiatric dysfunction, organ transplantation complication, osteoarthritis, osteoporosis, Parkinson's disease, pre-eclampsia, primary biliary cirrhosis, progerias, prostatic hypertrophy, psoriasis, pulmonary disease, radiation complication, renal dysfunction, urinary incontinence, vascular calcification, or vascular hyporeactivity.

81. The method of any one of claims 77-79, wherein the disease is cancer.

82. A method of selectively killing one or more cancer cells in a subject in need thereof comprising administering to the subject an effective amount of a compound of any one of claims 1-64, or a pharmaceutically acceptable salt, solvate, hydrate, polymorph, co-crystal, tautomer, stereoisomer, isotopically labeled compound, or prodrug thereof, or a pharmaceutical composition of any one of claims 66-69.

83. The method of any one of claims 73-76 and 81-82, wherein the cancer is a Bcl-2-mediated cancer.

84. The method of any one of claims 73-76 and 81-83, wherein the cancer is a Bcl-xL-mediated cancer.

85. The method of any one of claims 73-76 and 81-84, wherein the cancer is a hematological malignancy.

86. The method of claim 85, wherein the cancer is leukemia.

87. The method of claim 85, wherein the cancer is acute myeloid leukemia, chronic lymphocytic leukemia, or acute lymphoblastic leukemia.

88. The method of claim 85, wherein the cancer is lymphoma.

89. The method of claim 85, wherein the cancer is T-cell lymphoma.

90. The method of any one of claims 73-76 and 81-84, wherein the cancer is a solid tumor.

91. The method of any one of claims 76-90, wherein the subject is a mammal.

92. The method of claim 91, wherein the subject is a human.
